(19) United States
(12) Patent Application Publication
(10) Pub. No.: US 2026/0265207 A1
(43) Pub. Date: Sep. 10, 2026

(54) DPP1 INHIBITORS WITH POLYCYCLIC LINKERS AND USES THEREOF

(71) Applicants: Adam J. PLAUNT, Bridgewater, NJ (US); Insmed Incorporated, Bridgewater, NJ (US)

(72) Inventor: Adam J. PLAUNT, Bridgewater, NJ (US)

(73) Assignee: Insmed Incorporated, Bridgewater, NJ (US)

(21) Appl. No.: 19/165,462

(22) PCT Filed: Mar. 15, 2024
(86) PCT No.: PCT/US2024/020305

§ 371 Date: Sep. 15, 2025

Related U.S. Application Data
(60) Provisional application No. 63/452,636, filed on Mar. 16, 2023.
(60) Provisional application No. 63/546,686, filed on Oct. 31, 2023.

(51) Int. Cl. C07D 267/10; A61K 31/55; A61K 31/553; C07D 223/04; C07D 413/12; C07D 413/14; C07D 495/04

(52) U.S. Cl. CPC C07D 267/10; A61K 31/55; A61K 31/553; C07D 223/04; C07D 413/12; C07D 413/14; C07D 495/04

(57) ABSTRACT

Provided herein are compounds of Formula (I), or pharmaceutically acceptable salts or deuterated forms thereof, wherein R⁰, L and R¹ are defined herein. Also provided herein are pharmaceutical compositions comprising a compound of Formula (I) or pharmaceutically acceptable salt or deuterated form thereof, and methods of using a compound of Formula (I) or pharmaceutically acceptable salt or deuterated form thereof, e.g., in the treatment of a disease that is treatable by administration of a DPP1 inhibitor.

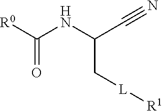

### CROSS REFERENCE TO RELATED APPLICATIONS

This application claims priority to U.S. Provisional Application No. 63/452,636 filed Mar. 16, 2023, and U.S. Provisional Application No. 63/546,686, filed Oct. 31, 2023, the contents of which are hereby incorporated by reference in their entities for all purposes.

### BACKGROUND

Dipeptidyl peptidase 1 (DPP1; EC 3.4.14.1), also known as cathepsin C, is a lysosomal cysteine protease belonging to the papain family having a molecular weight of 200 kDa. DPP1 was first discovered by Gutman and Fruton in 1948 (J Biol Chem, 174, 851-858); however, the cDNA of the human enzyme was first described in 1995 (Paris et al. 1995, FEBS Lett, 369, 326-330). DPP1 is the only member of the papain family that is functional as a tetramer, consisting of four identical subunits. Each subunit is composed of an N-terminal fragment, a heavy chain and a light chain (Dolenc et al. 1995, J Biol Chem, 270, 21626-21631).

DPP1 is constitutively expressed in many tissues with highest levels in lung, kidney, liver and spleen. DPP1 catalyzes the removal of dipeptides from the N-terminal end of polypeptide substrates with broad specificity. Recent data suggest that besides being an important enzyme in lysosomal protein degradation, DPP1 also functions as a key enzyme in the activation of granule serine proteases in cytotoxic T-lymphocytes and natural killer cells (granzymes A and B), mast cells (chymase and tryptase) and neutrophils (cathepsin G, neutrophil elastase and proteinase-3).

Mast cells are found in many tissues but are present in greater numbers along the epithelial linings of the body, such as the skin, respiratory tract and gastrointestinal tract. In humans, two types of mast cells have been identified. The T-type, which expresses only tryptase, and the MC-type, which expresses both tryptase and chymase. In humans, the T-type mast cells are located primarily in alveolar tissue and intestinal mucosa while the TC-type cells predominate in skin and conjunctiva. Tryptase and chymase appear to be important mediators of allergic diseases, being involved in processes of inflammation, bronchoconstriction and mucus secretion.

Neutrophils play a critical role in host defense against invading pathogens. Neutrophils are produced in the bone marrow and are fully mature when released into the circulation to take up their role as the first line of cellular defense. Pro-inflammatory mediators and chemotactic attractants activate neutrophils and draw them to the site of infection, where they act to engulf bacteria by phagocytosis, assaulting them with an arsenal of anti-bacterial compounds that use both oxidative and non-oxidative methods of attack. The powerful serine protease, neutrophil elastase, is one of those anti-bacterial compounds that are clearly involved in destroying bacteria. Neutrophil elastase is released into the phagolysome surrounding the microorganism, which it proceeds to destroy. Neutrophil elastase is able to attack the outer membrane protein, OmpA, in gram-negative bacteria, helping to directly kill the pathogen by degrading its membrane, as well as enabling other anti-bacterial compounds to gain access to the pathogen. In addition, neutrophil elastase may help process other antibacterial compounds, converting them from inactive pro-peptides into their active states, such as for cathelicidin.

Yet neutrophil elastase can also cause problems for its host. It is one of the most destructive enzymes in the body, with the capability of degrading extracellular matrix proteins (including collagens, proteoglycan, fibronectin, platelet receptors, complement receptor, thrombomodulin, lung surfactant and cadherins) and key plasma proteins (including coagulation and complement factors, immunoglobulin, several proteases and protease inhibitors). Under physiological conditions, endogenous protease inhibitors, such as al-antitrypsin, tightly regulate the activity of neutrophil elastase. However, at inflammatory sites, neutrophil elastase is able to evade regulation, and once unregulated it can induce the release of pro-inflammatory cytokines, such as interleukin-6 and interleukin-8, leading to acute lung injury. It can even impair host defense against infection by degrading phagocyte surface receptors and opsonins. Its negative role is illustrated by its involvement in the tissue destruction and inflammation that characterize numerous diseases, including hereditary emphysema, chronic obstructive pulmonary disease, cystic fibrosis, adult respiratory distress syndrome, ischemic-reperfusion injury and rheumatoid arthritis.

As such, there is a need in the art to provide novel DPP1 inhibitors in order to treat the aforementioned diseases, and others associated with DPP1 and neutrophil elastase.

### SUMMARY

In some aspects, the presents disclosure provides a compound of formula (I),

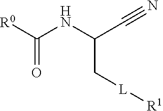

or a pharmaceutically acceptable salt or deuterated form thereof,
wherein:
R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
L is polycyclic cycloalkylene, polycyclic arylene or polycyclic heteroarylene, wherein a first atom of a first ring of the polycyclic arylene or polycyclic heteroarylene is connected to

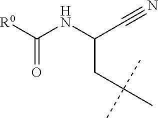

and a second atom of a second ring of the polycyclic arylene or heteroarylene is connected to R¹;
wherein L is independently substituted by 0-4 R¹⁰;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH;
R¹ is
   5-12 membered carbocyclyl optionally substituted with 1-3 R^{g},
   6-18 membered aryl optionally substituted with 1-3 R^{g},
   5-12 membered monocyclic heterocyclyl containing 1-3 heteroatoms selected from N, S or O, wherein the monocyclic heterocyclyl is optionally substituted with 1-3 R^{g},
5-12 membered monocyclic heteroaryl containing 1-3 heteroatoms selected from N, S or O, wherein the monocyclic heteroaryl is optionally substituted with 1-3 R^{g},
7-14 membered bicyclic heteroaryl containing 1-3 heteroatoms selected from N, S or O, wherein the bicyclic heteroaryl is optionally substituted with 1-4 R^{g}, or
   7-20 membered tricyclic heteroaryl containing 1-3 heteroatoms selected from N, S or O, wherein the tricyclic heteroaryl is optionally substituted with 1-5 R^{g},
each R^{g} is independently selected from hydrogen, SF₅, ═O, halogen, cyano, hydroxyl, nitro, NH₂, —COOH, C₁₋₆ alkyl, C₁₋₆ alkoxy, C₃₋₆ cycloalkyloxy, C₂₋₆alkenyloxy, C₂₋₆ alkynyloxy, C₃₋₆ cycloalkyl, C₂₋₆ alkenyl, C₂₋₅ alkynyl, —NHC₁₋₆ alkyl, —N(C₁₋₆ alkyl)₂, —COC₁₋₆alkyl, —COOC₁₋₆ alkyl, —CONH₂, —CONHC₁₋₆ alkyl, —CONHC₃₋₆ cycloalkyl, —CON(C₁₋₆ alkyl)₂, —NHCOC₁₋₆alkyl, —NHCOC₃₋₅cycloalkyl, —P(O)(C₁₋₆ alkyl), —S(O)C₁₋₆alkyl, —S(O)₂C₁₋₆ alkyl, —S(O)₂C₃₋₆cycloalkyl, —SO₂-3-7 membered heterocyclyl, —S(O)NH₂, —S(O)NHC₁₋₆ alkyl, —S(O)N(C₁₋₆ alkyl)₂, —S(O)₂NH₂, —OSO₂—C₁₋₆alkyl, C₁₋₆alkylene-O—C₁₋₆alkyl, C₃₋₈-cycloalkenyloxy, aryl, heteroaryl, 3-7-membered heterocycle containing 1-3 heteroatoms selected from N, S and O, and the R^{g} is optionally further substituted with 1-3 groups selected from C₁₋₆ alkyl, C₁₋₆ haloalkyl, C₁₋₆-alkylene-OH, —CONH₂, NH₂, C₁₋₆ alkoxy, hydroxyl, —COOH, halogen, or a 5-7-membered heterocycle containing 1-3 heteroatoms selected from N, S and O, wherein the the 5-7-membered heterocycle is optionally substituted with 1-2 groups selected from ═O, halogen, cyano, C₁₋₆ alkyl, and C₁₋₆ haloalkyl.

In embodiments, the present disclosure provides a compound of Formula (II):

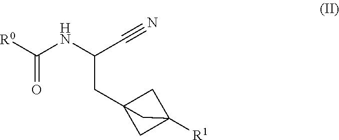

or a pharmaceutically acceptable salt or deuterated form thereof, wherein
R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
R¹ is
   5-12 membered carbocyclyl optionally substituted with 1-3 R^{g},
   6-18 membered aryl optionally substituted with 1-3 R^{g},
   5-12 membered monocyclic heterocyclyl containing 1-3 heteroatoms selected from N, S or O, wherein the monocyclic heterocyclyl is optionally substituted with 1-3 R^{g},
   5-12 membered monocyclic heteroaryl containing 1-3 heteroatoms selected from N, S or O, wherein the monocyclic heteroaryl is optionally substituted with 1-3 R^{g},
   7-14 membered bicyclic heteroaryl containing 1-3 heteroatoms selected from N, S or O, wherein the bicyclic heteroaryl is optionally substituted with 1-4 R^{g}, or
   7-20 membered tricyclic heteroaryl containing 1-3 heteroatoms selected from N, S or O, wherein the tricyclic heteroaryl is optionally substituted with 1-5 R^{g},
each R^{g} is independently selected from hydrogen, SF₅, ═O, halogen, cyano, hydroxyl, nitro, NH₂, —COOH, C₁₋₆ alkyl, C₁₋₆ alkoxy, C₃₋₆ cycloalkyloxy, C₂₋₆alkenyloxy, C₂₋₆ alkynyloxy, C₃₋₆ cycloalkyl, C₂₋₆ alkenyl, C₂₋₅ alkynyl, —NHC₁₋₆ alkyl, —N(C₁₋₆ alkyl)₂, —COC₁₋₆alkyl, —COOC₁₋₆ alkyl, —CONH₂, —CONHC₁₋₆ alkyl, —CONHC₃₋₆ cycloalkyl, —CON(C₁₋₆ alkyl)₂, —NHCOC₁₋₆alkyl, —NHCOC₃₋₅cycloalkyl, —P(O)(C₁₋₆ alkyl), —S(O)C₁₋₆alkyl, —S(O)₂C₁₋₆ alkyl, —S(O)₂C₃₋₆cycloalkyl, —SO₂-3-7 membered heterocyclyl, —S(O)NH₂, —S(O)NHC₁₋₆ alkyl, —S(O)N(C₁₋₆ alkyl)₂, —S(O)₂NH₂, —OSO₂—C₁₋₆alkyl, C₁₋₆alkylene-O—C₁₋₆alkyl, C₃₋₈-cycloalkenyloxy, aryl, heteroaryl, 3-7-membered heterocycle containing 1-3 heteroatoms selected from N, S and O, and the R^{g} is optionally further substituted with 1-3 groups selected from C₁₋₆ alkyl, C₁₋₆ haloalkyl, C₁₋₆-alkylene-OH, —CONH₂, NH₂, C₁₋₆ alkoxy, hydroxyl, —COOH, halogen, or a 5-7-membered heterocycle containing 1-3 heteroatoms selected from N, S and O, wherein the the 5-7-membered heterocycle is optionally substituted with 1-2 groups selected from ═O, halogen, cyano, C₁₋₆ alkyl, and C₁₋₆ haloalkyl.

In embodiments, the present disclosure provides a compound of Formula (III):

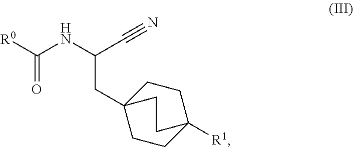

or a pharmaceutically acceptable salt or deuterated form thereof, wherein
R⁰ is
5-12 membered monocyclic heterocyclyl containing 1-3 heteroatoms selected from N, S, O, wherein the monocyclic heterocycle is optionally substituted with 1-3 R⁸, or
5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
R¹ is
5-12 membered carbocyclyl optionally substituted with 1-3 R^{g},
6-18 membered aryl optionally substituted with 1-3 R^{g},
5-12 membered monocyclic heterocyclyl containing 1-3 heteroatoms selected from N, S or O, wherein the monocyclic heterocyclyl is optionally substituted with 1-3 R^{g},
5-12 membered monocyclic heteroaryl containing 1-3 heteroatoms selected from N, S or O, wherein the monocyclic heteroaryl is optionally substituted with 1-3 R^{g},
7-14 membered bicyclic heteroaryl containing 1-3 heteroatoms selected from N, S or O, wherein the bicyclic heteroaryl is optionally substituted with 1-4 R^{g}, or
7-20 membered tricyclic heteroaryl containing 1-3 heteroatoms selected from N, S or O, wherein the tricyclic heteroaryl is optionally substituted with 1-5 R^{g},
each R^{g} is independently selected from hydrogen, SF₅, ═O, halogen, cyano, hydroxyl, nitro, NH₂, —COOH, C₁₋₆ alkyl, C₁₋₆ alkoxy, C₃₋₆ cycloalkyloxy, C₂₋₆alkenyloxy, C₂₋₆ alkynyloxy, C₃₋₆ cycloalkyl, C₂₋₆ alkenyl, C₂₋₅ alkynyl, —NHC₁₋₆ alkyl, —N(C₁₋₆ alkyl)₂, —COC₁₋₆alkyl, —COOC₁₋₆ alkyl, —CONH₂, —CONHC₁₋₆ alkyl, —CONHC₃₋₆ cycloalkyl, —CON(C₁₋₆ alkyl)₂, —NHCOC₁₋₆alkyl, —NHCOC₃₋₅cycloalkyl, —P(O)(C₁₋₆ alkyl), —S(O)C₁₋₆alkyl, —S(O)₂C₁₋₆ alkyl, —S(O)₂C₃₋₆cycloalkyl, —SO₂-3-7 membered heterocyclyl, —S(O)NH₂, —S(O)N(C₁₋₆ alkyl, —S(O)N(C₁₋₆ alkyl)₂, —S(O)₂NH₂, —OSO₂—C₁₋₆alkyl, C₁₋₆alkylene-O—C₁₋₆alkyl, C₃₋₈-cycloalkenyloxy, aryl, heteroaryl, 3-7-membered heterocycle containing 1-3 heteroatoms selected from N, S and O, and the R^{g} is optionally further substituted with 1-3 groups selected from C₁₋₆ alkyl, C₁₋₆ haloalkyl, C₁₋₆-alkylene-OH, —CONH₂, NH₂, C₁₋₆ alkoxy, hydroxyl, —COOH, halogen, or a 5-7-membered heterocycle containing 1-3 heteroatoms selected from N, S and O, wherein the the 5-7-membered heterocycle is optionally substituted with 1-2 groups selected from ═O, halogen, cyano, C₁₋₆ alkyl, and C₁₋₆ haloalkyl.

In embodiments, the present disclosure provides a compound of Formula (IV):

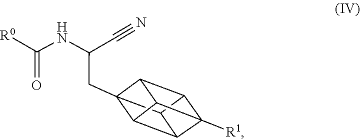

or a pharmaceutically acceptable salt or deuterated form thereof, wherein
R⁰ is
5-12 membered monocyclic heterocyclyl containing 1-3 heteroatoms selected from N, S, O, wherein the monocyclic heterocycle is optionally substituted with 1-3 R⁸, or
5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
R¹ is
5-12 membered carbocyclyl optionally substituted with 1-3 R^{g},
6-18 membered aryl optionally substituted with 1-3 R^{g},
5-12 membered monocyclic heterocyclyl containing 1-3 heteroatoms selected from N, S or O, wherein the monocyclic heterocyclyl is optionally substituted with 1-3 R^{g},
5-12 membered monocyclic heteroaryl containing 1-3 heteroatoms selected from N, S or O, wherein the monocyclic heteroaryl is optionally substituted with 1-3 R^{g},
7-14 membered bicyclic heteroaryl containing 1-3 heteroatoms selected from N, S or O, wherein the bicyclic heteroaryl is optionally substituted with 1-4 R^{g}, or
7-20 membered tricyclic heteroaryl containing 1-3 heteroatoms selected from N, S or O, wherein the tricyclic heteroaryl is optionally substituted with 1-5 R^{g},
each R^{g} is independently selected from hydrogen, SF₅, ═O, halogen, cyano, hydroxyl, nitro, NH₂, —COOH, C₁₋₆ alkyl, C₁₋₆ alkoxy, C₃₋₆ cycloalkyloxy, C₂₋₆alkenyloxy, C₂₋₆ alkynyloxy, C₃₋₆ cycloalkyl, C₂₋₆ alkenyl, C₂₋₅ alkynyl, —NHC₁₋₆ alkyl, —N(C₁₋₆ alkyl)₂, —COC₁₋₆alkyl, —COOC₁₋₆ alkyl, —CONH₂, —CONHC₁₋₆ alkyl, —CONHC₃₋₆ cycloalkyl, —CON(C₁₋₆ alkyl)₂, —NHCOC₁₋₆alkyl, —NHCOC₃₋₅cycloalkyl, —P(O)(C₁₋₆ alkyl), —S(O)C₁₋₆alkyl, —S(O)₂C₁₋₆ alkyl, —S(O)₂C₃₋₆cycloalkyl, —SO₂-3-7 membered heterocyclyl, —S(O)NH₂, —S(O)NHC₁₋₆ alkyl, —S(O)N(C₁₋₆ alkyl)₂, —S(O)₂NH₂, —OSO₂—C₁₋₆alkyl, C₁₋₆alkylene-O—C₁₋₆alkyl, C₃₋₈-cycloalkenyloxy, aryl, heteroaryl, 3-7-membered heterocycle containing 1-3 heteroatoms selected from N, S and O, and the R^{g} is optionally further substituted with 1-3 groups selected from C₁₋₆ alkyl, C₁₋₆ haloalkyl, C₁₋₆-alkylene-OH, —CONH₂, NH₂, C₁₋₆ alkoxy, hydroxyl, —COOH, halogen, or a 5-7-membered heterocycle containing 1-3 heteroatoms selected from N, S and O, wherein the the 5-7-membered heterocycle is optionally substituted with 1-2 groups selected from ═O, halogen, cyano, C₁₋₆ alkyl, and C₁₋₆ haloalkyl.

In embodiments, the present disclosure provides a compound of Formula (V),

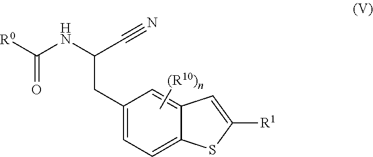

or a pharmaceutically acceptable salt or deuterated form thereof, wherein
R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
R¹ is
   5-12 membered carbocyclyl optionally substituted with 1-3 R^{g},
   6-18 membered aryl optionally substituted with 1-3 R^{g},
   5-12 membered monocyclic heterocyclyl containing 1-3 heteroatoms selected from N, S or O, wherein the monocyclic heterocyclyl is optionally substituted with 1-3 R^{g},
   5-12 membered monocyclic heteroaryl containing 1-3 heteroatoms selected from N, S or O, wherein the monocyclic heteroaryl is optionally substituted with 1-3 R^{g},
   7-14 membered bicyclic heteroaryl containing 1-3 heteroatoms selected from N, S or O, wherein the bicyclic heteroaryl is optionally substituted with 1-4 R^{g}, or
   7-20 membered tricyclic heteroaryl containing 1-3 heteroatoms selected from N, S or O, wherein the tricyclic heteroaryl is optionally substituted with 1-5 R^{g},
each R^{g} is independently selected from hydrogen, SF₅, ═O, halogen, cyano, hydroxyl, nitro, NH₂, —COOH, C₁₋₆ alkyl, C₁₋₆ alkoxy, C₃₋₆ cycloalkyloxy, C₂₋₆alkenyloxy, C₂₋₆ alkynyloxy, C₃₋₆ cycloalkyl, C₂₋₆ alkenyl, C₂₋₅ alkynyl, —NHC₁₋₆ alkyl, —N(C₁₋₆ alkyl)₂, —COC₁₋₆alkyl, —COOC₁₋₆ alkyl, —CONH₂, —CONHC₁₋₆ alkyl, —CONHC₃₋₆ cycloalkyl, —CON(C₁₋₆ alkyl)₂, —NHCOC₁₋₆alkyl, —NHCOC₃₋₅cycloalkyl, —P(O)(C₁₋₆ alkyl), —S(O)C₁₋₆alkyl, —S(O)₂C₁₋₆ alkyl, —S(O)₂C₃₋₆cycloalkyl, —SO₂-3-7 membered heterocyclyl, —S(O)NH₂, —S(O)NH—C₁₋₆ alkyl, —S(O)N(C₁₋₆ alkyl)₂, —S(O)₂NH₂, —OSO₂—C₁₋₆alkyl, C₁₋₆alkylene-O—C₁₋₆alkyl, C₃₋₈-cycloalkenyloxy, aryl, heteroaryl, 3-7-membered heterocycle containing 1-3 heteroatoms selected from N, S and O, and the R^{g} is optionally further substituted with 1-3 groups selected from C₁₋₆ alkyl, C₁₋₆ haloalkyl, C₁₋₆-alkylene-OH, —CONH₂, NH₂, C₁₋₆ alkoxy, hydroxyl, —COOH, halogen, or a 5-7-membered heterocycle containing 1-3 heteroatoms selected from N, S and O, wherein the the 5-7-membered heterocycle is optionally substituted with 1-2 groups selected from ═O, halogen, cyano, C₁₋₆ alkyl, and C₁₋₆ haloalkyl,
   n is 0 or 1;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH, and R¹⁰ is substituted on the phenyl ring or the thiophenyl ring of the benzothienylene ring.

In embodiments, the present disclosure provides a compound of formula (VI),

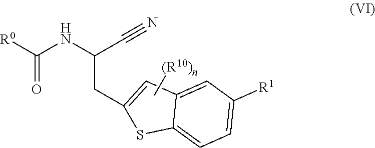

or a pharmaceutically acceptable salt or deuterated form thereof, wherein
R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
R¹ is
   5-12 membered carbocyclyl optionally substituted with 1-3 R^{g},
   6-18 membered aryl optionally substituted with 1-3 R^{g},
   5-12 membered monocyclic heterocyclyl containing 1-3 heteroatoms selected from N, S or O, wherein the monocyclic heterocyclyl is optionally substituted with 1-3 R^{g},
   5-12 membered monocyclic heteroaryl containing 1-3 heteroatoms selected from N, S or O, wherein the monocyclic heteroaryl is optionally substituted with 1-3 R^{g},
   7-14 membered bicyclic heteroaryl containing 1-3 heteroatoms selected from N, S or O, wherein the bicyclic heteroaryl is optionally substituted with 1-4 R^{g}, or
   7-20 membered tricyclic heteroaryl containing 1-3 heteroatoms selected from N, S or O, wherein the tricyclic heteroaryl is optionally substituted with 1-5 R^{g},
each R^{g} is independently selected from hydrogen, SF₅, ═O, halogen, cyano, hydroxyl, nitro, NH₂, —COOH, C₁₋₆ alkyl, C₁₋₆ alkoxy, C₃₋₆ cycloalkyloxy, C₂₋₆alkenyloxy, C₂₋₆ alkynyloxy, C₃₋₆ cycloalkyl, C₂₋₆ alkenyl, C₂₋₅ alkynyl, —NHC₁₋₆ alkyl, —N(C₁₋₆ alkyl)₂, —COC₁₋₆alkyl, —COOC₁₋₆ alkyl, —CONH₂, —CONHC₁₋₆ alkyl, —CONHC₃₋₆ cycloalkyl, —CON(C₁₋₆ alkyl)₂, —NHCOC₁₋₆alkyl, —NHCOC₃₋₅cycloalkyl, —P(O)(C₁₋₆ alkyl), —S(O)C₁₋₆alkyl, —S(O)₂C₁₋₆ alkyl, —S(O)₂C₃₋₆cycloalkyl, —SO₂-3-7 membered heterocyclyl, —S(O)NH₂, —S(O)NHC₁₋₆ alkyl, —S(O)N(C₁₋₆ alkyl)₂, —S(O)₂NH₂, —OSO₂—C₁₋₆alkyl, C₁₋₆alkylene-O—C₁₋₆alkyl, C₃₋₈-cycloalkenyloxy, aryl, heteroaryl, 3-7-membered heterocycle containing 1-3 heteroatoms selected from N, S and O, and the R^{g} is optionally further substituted with 1-3 groups selected from C₁₋₆ alkyl, C₁₋₆ haloalkyl, C₁₋₆-alkylene-OH, —CONH₂, NH₂, C₁₋₆ alkoxy, hydroxyl, —COOH, halogen, or a 5-7-membered heterocycle containing 1-3 heteroatoms selected from N, S and O, wherein the the 5-7-membered heterocycle is optionally substituted with 1-2 groups selected from ═O, halogen, cyano, C₁₋₆ alkyl, and C₁₋₆ haloalkyl,
n is 0 or 1;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH, and R¹⁰ is substituted on the phenyl ring or the thiophenyl ring of the benzothienylene ring.

In embodiments, the present disclosure provides a compound of formula (VII),

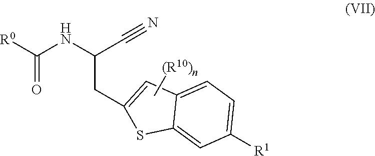

or a pharmaceutically acceptable salt or deuterated form thereof, wherein
R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
R¹ is
   5-12 membered carbocyclyl optionally substituted with 1-3 R^{g},
   6-18 membered aryl optionally substituted with 1-3 R^{g},
   5-12 membered monocyclic heterocyclyl containing 1-3 heteroatoms selected from N, S or O, wherein the monocyclic heterocyclyl is optionally substituted with 1-3 R^{g},
   5-12 membered monocyclic heteroaryl containing 1-3 heteroatoms selected from N, S or O, wherein the monocyclic heteroaryl is optionally substituted with 1-3 R^{g},
   7-14 membered bicyclic heteroaryl containing 1-3 heteroatoms selected from N, S or O, wherein the bicyclic heteroaryl is optionally substituted with 1-4 R^{g}, or
   7-20 membered tricyclic heteroaryl containing 1-3 heteroatoms selected from N, S or O, wherein the tricyclic heteroaryl is optionally substituted with 1-5 R^{g},
each R^{g} is independently selected from hydrogen, SF₅, ═O, halogen, cyano, hydroxyl, nitro, NH₂, —COOH, C₁₋₆ alkyl, C₁₋₆ alkoxy, C₃₋₆ cycloalkyloxy, C₂₋₆alkenyloxy, C₂₋₆ alkynyloxy, C₃₋₆ cycloalkyl, C₂₋₆ alkenyl, C₂₋₅ alkynyl, —NHC₁₋₆ alkyl, —N(C₁₋₆ alkyl)₂, —COC₁₋₆alkyl, —COOC₁₋₆ alkyl, —CONH₂, —CONHC₁₋₆ alkyl, —CONHC₃₋₆ cycloalkyl, —CON(C₁₋₆ alkyl)₂, —NHCOC₁₋₆alkyl, —NHCOC₃₋₅cycloalkyl, —P(O)(C₁₋₆ alkyl), —S(O)C₁₋₆alkyl, —S(O)₂C₁₋₆ alkyl, —S(O)₂C₃₋₆cycloalkyl, —SO₂-3-7 membered heterocyclyl, —S(O)NH₂, —S(O)NHC₁₋₆ alkyl, —S(O)N(C₁₋₆ alkyl)₂, —S(O)₂NH₂, —OSO₂—C₁₋₆alkyl, C₁₋₆alkylene-O—C₁₋₆alkyl, C₃₋₈-cycloalkenyloxy, aryl, heteroaryl, 3-7-membered heterocycle containing 1-3 heteroatoms selected from N, S and O, and the R^{g} is optionally further substituted with 1-3 groups selected from C₁₋₆ alkyl, C₁₋₆ haloalkyl, C₁₋₆-alkylene-OH, —CONH₂, NH₂, C₁₋₆ alkoxy, hydroxyl, —COOH, halogen, or a 5-7-membered heterocycle containing 1-3 heteroatoms selected from N, S and O, wherein the the 5-7-membered heterocycle is optionally substituted with 1-2 groups selected from ═O, halogen, cyano, C₁₋₆ alkyl, and C₁₋₆ haloalkyl,
n is 0 or 1;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH, and R¹⁰ is substituted on the phenyl ring or the thiophenyl ring of the benzothienylene ring.

In embodiments, the present disclosure provides a compound of formula (XIV),

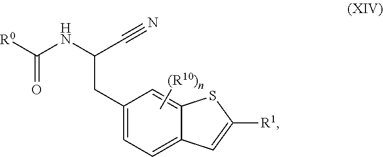

or a pharmaceutically acceptable salt or deuterated form thereof, wherein
R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
R¹ is
   5-12 membered carbocyclyl optionally substituted with 1-3 R^{g},
   6-18 membered aryl optionally substituted with 1-3 R^{g},
   5-12 membered monocyclic heterocyclyl containing 1-3 heteroatoms selected from N, S or O, wherein the monocyclic heterocyclyl is optionally substituted with 1-3 R^{g},
   5-12 membered monocyclic heteroaryl containing 1-3 heteroatoms selected from N, S or O, wherein the monocyclic heteroaryl is optionally substituted with 1-3 R^{g},
   7-14 membered bicyclic heteroaryl containing 1-3 heteroatoms selected from N, S or O, wherein the bicyclic heteroaryl is optionally substituted with 1-4 R^{g}, or
   7-20 membered tricyclic heteroaryl containing 1-3 heteroatoms selected from N, S or O, wherein the tricyclic heteroaryl is optionally substituted with 1-5 R^{g} groups,
each R^{g} is independently selected from hydrogen, SF₅, ═O, halogen, cyano, hydroxyl, nitro, NH₂, —COOH, C₁₋₆ alkyl, C₁₋₆ alkoxy, C₃₋₆ cycloalkyloxy, C₂₋₆alkenyloxy, C₂₋₆ alkynyloxy, C₃₋₆ cycloalkyl, C₂₋₆ alkenyl, C₂₋₅ alkynyl, —NHC₁₋₆ alkyl, —N(C₁₋₆ alkyl)₂, —COC₁₋₆alkyl, —COOC₁₋₆ alkyl, —CONH₂, —CONHC₁₋₆ alkyl, —CONHC₃₋₆ cycloalkyl, —CON(C₁₋₆ alkyl)₂, —NHCOC₁₋₆alkyl, —NHCOC₃₋₅cycloalkyl, —P(O)(C₁₋₆ alkyl), —S(O)C₁₋₆alkyl, —S(O)₂C₁₋₆ alkyl, —S(O)₂C₃₋₆cycloalkyl, —SO₂-3-7 membered heterocyclyl, —S(O)NH₂, —S(O)NHC₁₋₆ alkyl, —S(O)N(C₁₋₆ alkyl)₂, —S(O)₂NH₂, —OSO₂—C₁₋₆alkyl, C₁₋₆alkylene-O—C₁₋₆alkyl, C₃₋₈-cycloalkenyloxy, aryl, heteroaryl, 3-7-membered heterocycle containing 1-3 heteroatoms selected from N, S and O, and the R^{g} is optionally further substituted with 1-3 groups selected from C₁₋₆ alkyl, C₁₋₆ haloalkyl, C₁₋₆-alkylene-OH, —CONH₂, NH₂, C₁₋₆ alkoxy, hydroxyl, —COOH, halogen, or a 5-7-membered heterocycle containing 1-3 heteroatoms selected from N, S and O, wherein the the 5-7-membered heterocycle is optionally substituted with 1-2 groups selected from ═O, halogen, cyano, C₁₋₆ alkyl, and C₁₋₆ haloalkyl,
n is 0 or 1;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH, and R¹⁰ is substituted on the phenyl ring or the thiophenyl ring of the benzothienylene ring.

In embodiments, the present disclosure provides a compound of formula (VIII),

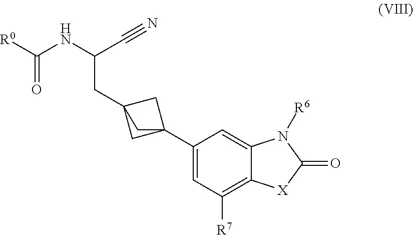

or a pharmaceutically acceptable salt or deuterated form thereof, wherein
R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
R¹ is
   5-12 membered carbocyclyl optionally substituted with 1-3 R^{g},
   6-18 membered aryl optionally substituted with 1-3 R^{g},
   5-12 membered monocyclic heterocyclyl containing 1-3 heteroatoms selected from N, S or O, wherein the monocyclic heterocyclyl is optionally substituted with 1-3 R^{g},
   5-12 membered monocyclic heteroaryl containing 1-3 heteroatoms selected from N, S or O, wherein the monocyclic heteroaryl is optionally substituted with 1-3 R^{g},
   7-14 membered bicyclic heteroaryl containing 1-3 heteroatoms selected from N, S or O, wherein the bicyclic heteroaryl is optionally substituted with 1-4 R^{g}, or
   7-20 membered tricyclic heteroaryl containing 1-3 heteroatoms selected from N, S or O, wherein the tricyclic heteroaryl is optionally substituted with 1-5 R^{g},
each R^{g} is independently selected from hydrogen, SF₅, ═O, halogen, cyano, hydroxyl, nitro, NH₂, —COOH, C₁₋₆ alkyl, C₁₋₆ alkoxy, C₃₋₆ cycloalkyloxy, C₂₋₆alkenyloxy, C₂₋₆ alkynyloxy, C₃₋₆ cycloalkyl, C₂₋₆ alkenyl, C₂₋₅ alkynyl, —NHC₁₋₆ alkyl, —N(C₁₋₆ alkyl)₂, —COC₁₋₆alkyl, —COOC₁₋₆ alkyl, —CONH₂, —CONHC₁₋₆ alkyl, —CONHC₃₋₆ cycloalkyl, —CON(C₁₋₆ alkyl)₂, —NHCOC₁₋₆alkyl, —NHCOC₃₋₅cycloalkyl, —P(O)(C₁₋₆ alkyl), —S(O)C₁₋₆alkyl, —S(O)₂C₁₋₆ alkyl, —S(O)₂C₃₋₆cycloalkyl, —SO₂-3-7 membered heterocyclyl, —S(O)NH₂, —S(O)NHC₁₋₆ alkyl, —S(O)N(C₁₋₆ alkyl)₂, —S(O)₂NH₂, —OSO₂—C₁₋₆alkyl, C₁₋₆alkylene-O—C₁₋₆alkyl, C₃₋₈-cycloalkenyloxy, aryl, heteroaryl, 3-7-membered heterocycle containing 1-3 heteroatoms selected from N, S and O, and the R^{g} is optionally further substituted with 1-3 groups selected from C₁₋₆ alkyl, C₁₋₆ haloalkyl, C₁₋₆-alkylene-OH, —CON-2, NH₂, C₁₋₆ alkoxy, hydroxyl, —COOH, halogen, or a 5-7-membered heterocycle containing 1-3 heteroatoms selected from N, S and O, wherein the the 5-7-membered heterocycle is optionally substituted with 1-2 groups selected from ═O, halogen, cyano, C₁₋₆ alkyl, and C₁₋₆ haloalkyl,
n is 0 or 1;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH.

In embodiments, the present disclosure provides a compound of formula (IX),

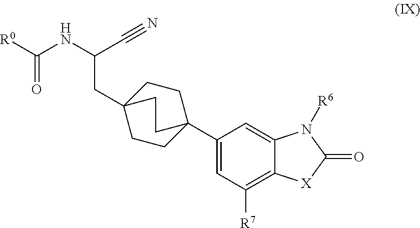

or a pharmaceutically acceptable salt or deuterated form thereof,
R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl; each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH; each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH.

In embodiments, the present disclosure provides a compound of formula (X),

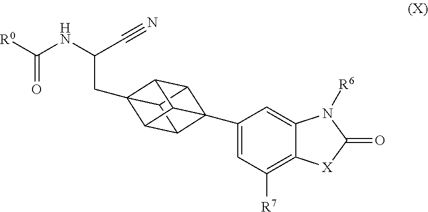

or a pharmaceutically acceptable salt or deuterated form thereof, wherein
R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH.

In embodiments, the present disclosure provides a compound of Formula (XI),

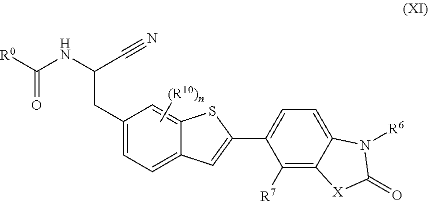

or a pharmaceutically acceptable salt or deuterated form thereof, wherein
R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₁₂, —S(O)₂NH₁₂, C₁₋₆alkoxy, halogenated C₁₋₆alkoxy, C₁₋₆ alkylene-carbocyclyl, or C₁₋₆ alkylene-heteroaryl;
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH,
n is 0 or 1;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NHI—C₁₋₆ alkyl, N(C₁₋₆alkyl)₂, COOH, COC₁₋₆ alkyl, COOC₁₋₆alkyl, CON₁₋₆alkyl, CON(C₁₋₆ alkyl)₂, NHCOC₁₋₆ alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH, and R¹⁰ is substituted on the phenyl ring or the thiophenyl ring of the benzothienylene ring.

In embodiments, the present disclosure provides a compound of Formula (XII),

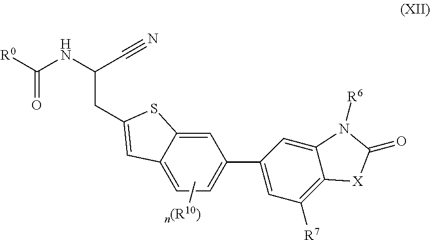

or a pharmaceutically acceptable salt or deuterated form thereof, wherein R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH,
n is 0 or 1;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH, and R¹⁰ is substituted on the phenyl ring or the thiophenyl ring of the benzothienylene ring.

In embodiments, the present disclosure provides a compound of Formula (XIII),

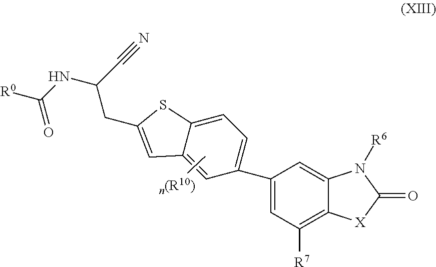

or a pharmaceutically acceptable salt or deuterated form thereof, wherein R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH,
n is 0 or 1;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH, and R¹⁰ is substituted on the phenyl ring or the thiophenyl ring of the benzothienylene ring.

In embodiments, the present disclosure provides a compound of Formula (XV),

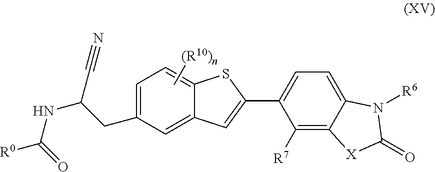

or a pharmaceutically acceptable salt or deuterated form thereof, wherein R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH,
n is 0 or 1;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH, and R¹⁰ is substituted on the phenyl ring or the thiophenyl ring of the benzothienylene ring.

In embodiments, the present disclosure provides a compound of Formula (XVI),

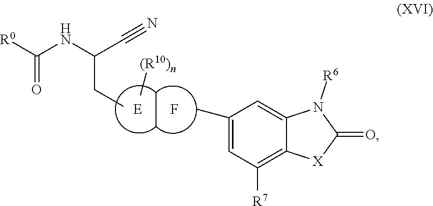

or a pharmaceutically acceptable salt or deuterated form thereof, wherein
R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH, each of ring E and ring F is a 5-membered heteroarylene ring;
n is 0 or 1;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH, and R¹⁰ is substituted on ring E or ring F.

In embodiments, the present disclosure provides a compound of Formula (XVII),

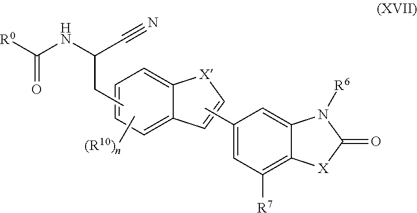

or a pharmaceutically acceptable salt or deuterated form thereof, wherein
R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH,
X¹ is O, S, NH or N(C₁₋₆ alkyl);
n is 0 or 1;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH, and R¹⁰ is substituted on the phenyl ring or the 5-membered ring of

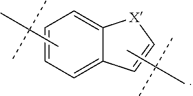

In embodiments, the present disclosure provides a compound of Formula (XVIII),

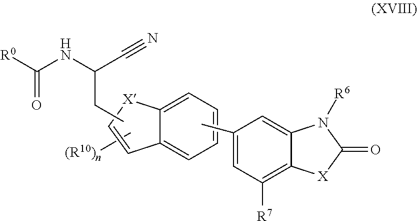

or a pharmaceutically acceptable salt or deuterated form thereof, wherein
R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH,
X¹ is O, S, NH or N(C₁₋₆ alkyl);
n is 0 or 1;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH, and R¹⁰ is substituted on the phenyl ring or the 5-membered ring of

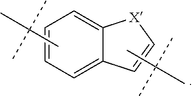

In embodiments, the present disclosure provides a compound of Formula (XIX),

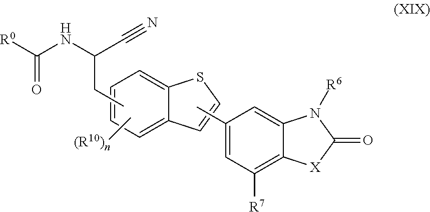

or a pharmaceutically acceptable salt or deuterated form thereof, wherein
R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH,
n is 0 or 1;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH, and R¹⁰ is substituted on the phenyl ring or the thiophene ring of

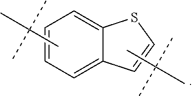

In embodiments, the present disclosure provides a compound of Formula (XX),

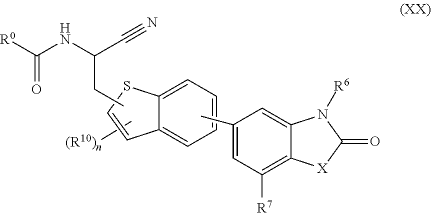

or a pharmaceutically acceptable salt or deuterated form thereof, wherein
R⁰ is
   5-12 membered monocyclic heterocyclyl containing 1-3 heteroatoms selected from N, S, O, wherein the monocyclic heterocycle is optionally substituted with 1-3 R⁸, or
   5-12-membered polycyclic heterocyclyl containing 1-3 heteroatoms selected from N, S, O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH,
n is 0 or 1;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH, and R¹⁰ is substituted on the phenyl ring or the thiophene ring of

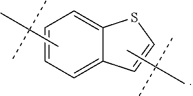

In embodiments, the present disclosure provides a pharmaceutical composition comprising a compound disclosed herein (e.g., a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX), or a pharmaceutically acceptable salt or deuterated form thereof), and a pharmaceutically acceptable adjuvant, diluent or carrier.

In yet another aspect of the disclosure, a method of treatment is provided. The method of treatment, in embodiments, comprises, administering to a subject in need thereof, a composition comprising an effective amount of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX), or a pharmaceutically acceptable salt or deuterated form thereof.

The method of treatment, in embodiments, is a method of treating an obstructive disease of the airway, e.g., cystic fibrosis (CF), asthma or bronchiectasis (e.g., non-CF bronchiectasis). In another embodiment, the method of treatment is a method for treating chronic rhinosinusitis (CRS).

In some embodiments, the method of treatment is a method for treating hidradenitis suppurativa (HS).

In some embodiments, the method of treatment is a method for treating cancer.

In some embodiments, the method of treatment is a method of treating lupus nephritis.

In some embodiments, the method of treatment is a method of treating rheumatoid arthritis.

In some embodiments, the method of treatment is a method of treating inflammatory bowel disease (IBD).

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

Listed below are definitions of various terms used in the specification and claims to describe the present disclosure.

Unless defined otherwise, all technical and scientific terms used in this disclosure have the same meanings as commonly understood by one of ordinary skill in the art to which this disclosure belongs.

The term “about” when immediately preceding a numerical value means a range encompassing said numerical value plus or minus an acceptable amount of variation in the art (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The terms below, as used herein, have the following meanings, unless indicated otherwise:

“Cyano” refers to the —CN radical.

“Hydroxy” or “hydroxyl” refers to the —OH radical.

“Oxo” refers to the ═O substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl. A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkenyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkynyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alkynyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused, spiro, or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}—R_{c} where R_{b} is an alkylene group as defined above and Re is one or more aryl radicals as defined above, for example, benzyl, diphenylmethyl and the like. Unless stated otherwise specifically in the specification, an aralkyl group can be optionally substituted.

“Carbocyclyl,” “carbocyclic ring” or “carbocycle” refers to a non-aromatic, saturated, unsaturated ring structure, wherein the atoms which form the ring are each carbon. Carbocyclic rings can comprise from 3 to 20 carbon atoms in the ring. Carbocyclic rings include cycloalkyl, cycloalkenyl and cycloalkynyl as defined herein. Unless stated otherwise specifically in the specification, a carbocyclyl group can be optionally substituted.

“Cycloalkyl” refers to a non-aromatic monocyclic or polycyclic fully saturated hydrocarbon radical consisting solely of carbon and hydrogen atoms, which can include fused, spiro, or bridged ring systems, having from three to twenty carbon atoms, e.g., having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Polycyclic” refers to ring systems comprising 2 or more rings, for example 2, 3, or 4 rings. Polycyclic rings may be fused, spiro, or bridged ring systems.

“Polycyclic cycloalkylene” refers to a divalent non-aromatic polycyclic fully saturated hydrocarbon ring consisting solely of carbon and hydrogen atoms, having from four to twenty carbon atoms, e.g., having from four to ten carbon atoms, and which is attached to the rest of the molecule (e.g., as shown in Formula I) by two single bonds. The polycyclic cycloalkylene can include fused, spiro, or bridged ring systems. Polycyclic cycloalkylene can include, for example, bicyclo[2.2.2]octanylene, cubanylene, bicyclo(1.1.1)pentylene, adamantylene, norbornylene, decalinylene, 7,7-dimethyl-bicyclo[2.2.1]heptanylene, and the like. Unless otherwise stated specifically in the specification, a cycloalkylene group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkynyl group can be optionally substituted.

“Heterocyclyl” “heterocyclic ring” or “heterocycle” refers to a 3- to 20-membered non-aromatic, saturated or unsaturated, radical which consists of two to twelve carbon atoms and from one to six heteroatoms selected from nitrogen, oxygen or sulfur. Unless stated otherwise specifically in the specification, the heterocyclyl radical can be a monocyclic, bicyclic, tricyclic or tetracyclic ring system, which can include fused, spiro, or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heterocyclylalkyl” refers to a radical of the formula —R_{b}—Rₑ where R_{b} is an alkylene group as defined above and Rₑ is a heterocyclyl radical as defined above. Unless stated otherwise specifically in the specification, a heterocycloalkyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical comprising hydrogen atoms, one to thirteen carbon atoms, one to six heteroatoms selected from nitrogen, oxygen and sulfur, and at least one aromatic ring. The heteroaryl radical can be a monocyclic, bicyclic, tricyclic or tetracyclic ring system, which can include fused or bridged ring systems; the nitrogen, carbon or sulfur atoms in the heteroaryl radical can be optionally oxidized; and the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophene), benzotriazolyl, benzo[4,6]imidazo[1,2-a]pyridinyl, carbazolyl, cinnolinyl, dibenzofuranyl, dibenzothiophene, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thienyl. Unless stated otherwise specifically in the specification, a heteroaryl group can be optionally substituted.

“Heteroarylene” refers to a divalent 5- to 20-membered ring system radical comprising hydrogen atoms, one to thirteen carbon atoms, one to six heteroatoms selected from nitrogen, oxygen and sulfur, and at least one aromatic ring comprising at least one heteroatom selected from nitrogen, oxygen and sulfur. For purposes of this disclosure, the heteroarylene radical can be a monocyclic, bicyclic, tricyclic or tetracyclic ring system, which can include fused, or bridged ring systems; and the nitrogen, carbon or sulfur atoms in the heteroaryl radical can be optionally oxidized; the nitrogen atom can be optionally quaternized. Examples include, but are not limited to, azepinylene, acridinylene, benzimidazolylene, benzothiazolylene, benzindolylene, benzodioxolylene, benzofuranylene, benzooxazolylene, benzothiazolylene, benzothiadiazolylene, benzo[b][1,4]dioxepinylene, 1,4-benzodioxanylene, benzonaphthofuranylene, benzoxazolylene, benzodioxolylene, benzodioxinylene, benzopyranylene, benzopyranonylene, benzofuranylene, benzofuranonylene, benzothienylene (divalent benzothiophene radical), benzotriazolylene, benzo[4,6]imidazo[1,2-a]pyridinylene, carbazolylene, cinnolinylene, dibenzofuranylene, dibenzothiophene, furanylene, furanonylene, isothiazolylene, imidazolylene, indazolylene, indolylene, indazolylene, isoindolylene, indolinylene, isoindolinylene, isoquinolylene, indolizinylene, isoxazolylene, naphthyridinylene, oxadiazolylene, 2-oxoazepinylene, oxazolylene, oxiranylene, 1-oxidopyridinylene, 1-oxidopyrimidinylene, 1-oxidopyrazinylene, 1-oxidopyridazinylene, 1-phenyl-1H-pyrrolylene, phenazinylene, phenothiazinylene, phenoxazinylene, phthalazinylene, pteridinylene, purinylene, pyrrolylene, pyrazolylene, pyridinylene, pyrazinylene, pyrimidinylene, pyridazinylene, quinazolinylene, quinoxalinylene, quinolinylene, quinuclidinylene, isoquinolinylene, tetrahydroquinolinylene, thiazolylene, thiadiazolylene, triazolylene, tetrazolylene, triazinylene, and thiophene (e.g., thienylene). Unless stated otherwise specifically in the specification, a heteroarylene group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (e.g., alkyl, alkylene, alkenyl, alkenylene, alkynyl, alkynylene, alkoxy, alkylamino, thioalkyl, aryl, aralkyl, carbocyclyl, cycloalkyl, cycloalkylene, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups.

“Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further includes any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

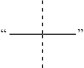

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

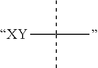

indicates that the chemical entity “XY” is bonded to another chemical entity via the point of attachment bond. Furthermore, the specific point of attachment to the non-depicted chemical entity can be specified by inference. For example, the compound CH₃—R^{L}, wherein R^{L} is H or

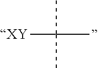

infers that when R^{L} is “XY”, the point of attachment bond is the same bond as the bond by which R^{L} is depicted as being bonded to CH₃.

In this specification, unless stated otherwise, the term “pharmaceutically acceptable” is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

The term “pharmaceutically acceptable salt” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The compounds of the invention, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

The term “treating” as used herein with regard to a patient, refers to improving at least one symptom of the patient's disorder. Treating can be improving, or at least partially ameliorating a disorder or an associated symptom of a disorder.

An “effective amount” means the amount compound or pharmaceutical formulation, that when administered to a patient for treating a state, disorder or condition is sufficient to effect such treatment.

The term “therapeutically effective” applied to dose or amount refers to that quantity of a compound or pharmaceutical formulation that is sufficient to result in a desired clinical benefit after administration to a patient in need thereof. A “therapeutically effective amount”, in some embodiments, is a dose or amount of a compound or pharmaceutical formulation that is sufficient to result in prophylaxis after administration to a patient in need thereof.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human.

### Compounds

In various embodiments, the disclosure provides DPP1 inhibitor of Formula (I), (II), (III), (IV), (V), (VI), (VII), (VIII), (IX), (X), (XI), (XII), (XIII), (XIV), (XV), (XVI), (XVII), (XVIII), (XIX) or (XX), or Table 1.

### Formulae

In some embodiments, the presents disclosure provides a compound of formula (I),

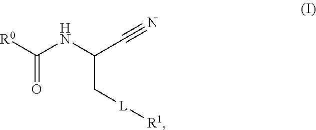

or a pharmaceutically acceptable salt or deuterated form thereof.

In embodiments, the present disclosure provides a compound of Formula (II):

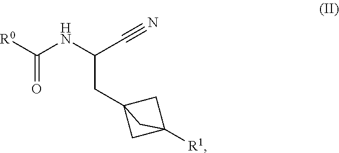

or a pharmaceutically acceptable salt or deuterated form thereof.

In embodiments, the present disclosure provides a compound of Formula (III):

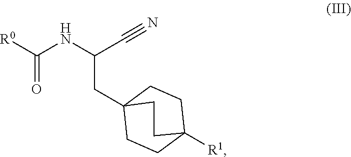

or a pharmaceutically acceptable salt or deuterated form thereof, wherein

In embodiments, the present disclosure provides a compound of Formula (IV):

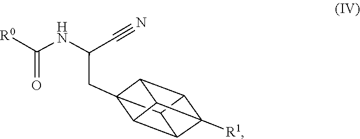

or a pharmaceutically acceptable salt or deuterated form thereof, wherein

In embodiments, the present disclosure provides a compound of Formula (V),

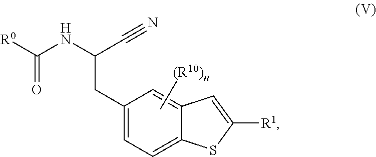

or a pharmaceutically acceptable salt or deuterated form thereof.

In embodiments, the present disclosure provides a compound of formula (VI),

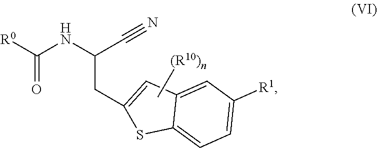

or a pharmaceutically acceptable salt or deuterated form thereof.

In embodiments, the present disclosure provides a compound of formula (VII),

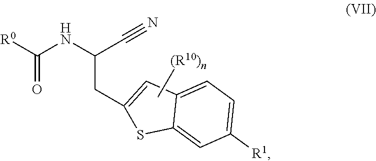

or a pharmaceutically acceptable salt or deuterated form thereof.

In embodiments, the present disclosure provides a compound of formula (XIV),

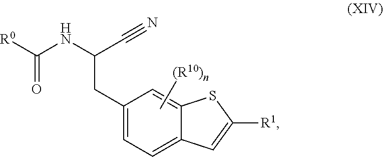

or a pharmaceutically acceptable salt or deuterated form thereof.

In embodiments, the present disclosure provides a compound of formula (VIII),

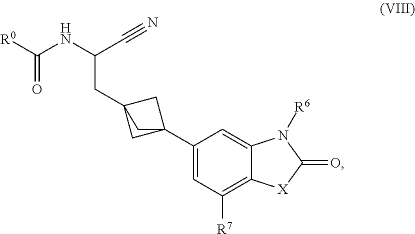

or a pharmaceutically acceptable salt or deuterated form thereof.

In embodiments, the present disclosure provides a compound of formula (IX),

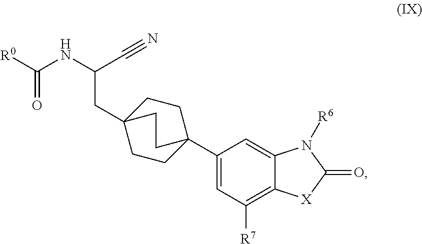

or a pharmaceutically acceptable salt or deuterated form thereof.

In embodiments, the present disclosure provides a compound of formula (X),

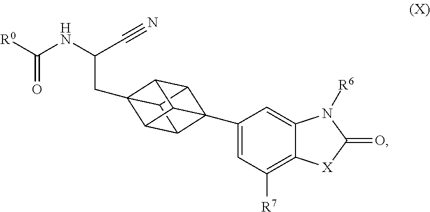

or a pharmaceutically acceptable salt or deuterated form thereof.

In embodiments, the present disclosure provides a compound of Formula (XI),

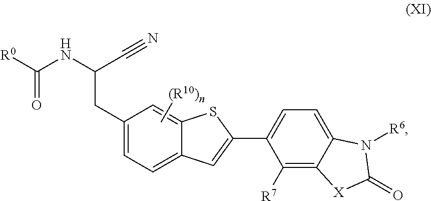

or a pharmaceutically acceptable salt or deuterated form thereof.

In embodiments, the present disclosure provides a compound of Formula (XII),

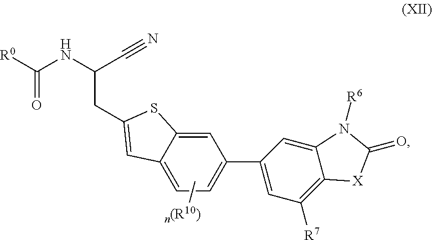

or a pharmaceutically acceptable salt or deuterated form thereof.

In embodiments, the present disclosure provides a compound of Formula (XIII),

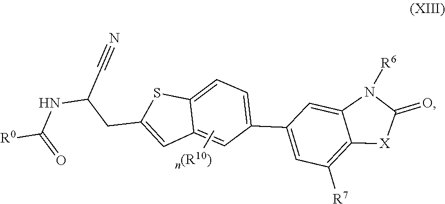

or a pharmaceutically acceptable salt or deuterated form thereof.

In embodiments, the present disclosure provides a compound of Formula (XV),

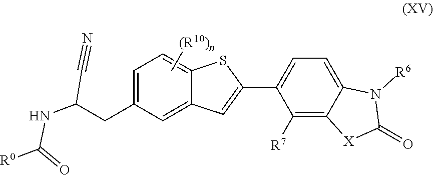

or a pharmaceutically acceptable salt or deuterated form thereof.

In embodiments, the present disclosure provides a compound of Formula (XVI),

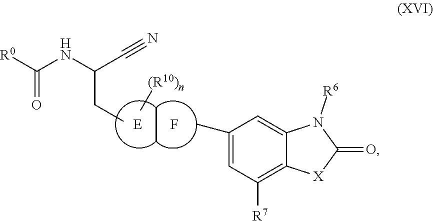

or a pharmaceutically acceptable salt or deuterated form thereof.

In embodiments, the present disclosure provides a compound of Formula (XVII),

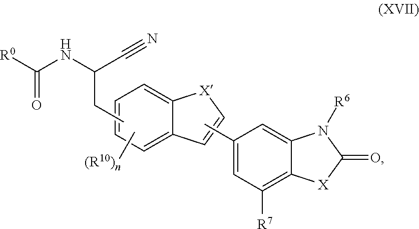

or a pharmaceutically acceptable salt or deuterated form thereof.

In embodiments, the present disclosure provides a compound of Formula (XVIII),

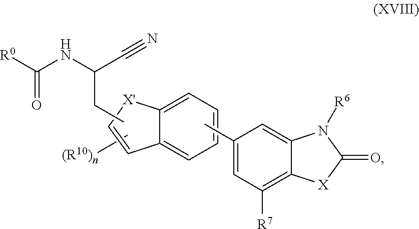

or a pharmaceutically acceptable salt or deuterated form thereof.

In embodiments, the present disclosure provides a compound of Formula (XIX),

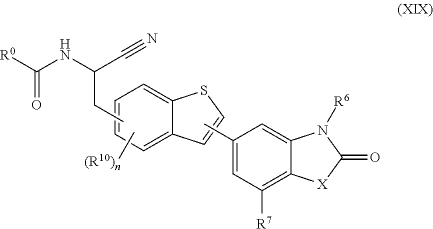

or a pharmaceutically acceptable salt or deuterated form thereof,

In embodiments, the present disclosure provides a compound of Formula (XX),

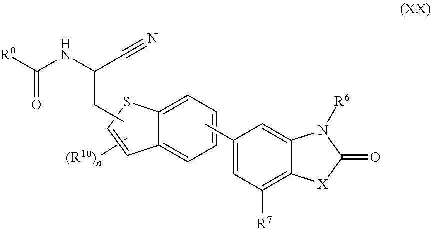

or a pharmaceutically acceptable salt or deuterated form thereof.

R⁰, L, R¹, R⁶, R⁸, R^{g}, R¹⁰, n, X, X′, X¹, X², X³, X⁴, R^{A}, R^{B}, ring B, ring C, ring D, Ring E and ring F of Formula (I), (II), (III), (IV), (V), (VI), (VII), (VIII), (IX), (X), (XI), (XII), (XIII), (XIV), (XV), (XVI), (XVII), (XVIII), (XIX) or (XX) are described herein.

### R⁰

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof or a deuterated form thereof, R⁰ is 5-12 membered monocyclic heterocyclyl containing 1-3 heteroatoms selected from N, S, O, wherein the monocyclic heterocycle is optionally substituted with 1-3 R⁸ groups, or 5-12-membered polycyclic heterocyclyl containing 1-3 heteroatoms selected from N, S, O, wherein the polycyclic heterocycle is optionally substituted with 1-3 R⁸ groups. In embodiments, each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁸ is H, OH, halogen, NH₂, COOH, C₁₋₆alkyl, C₁₋₆alkyl-OH, C₁₋₆alkoxy, or halogenated C₁₋₆alkoxy.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁸ is H, OH, halogen, NH₂, COOH, unsubstituted C₁₋₆alkyl, C₁₋₆alkyl-OH, unsubstituted C₁₋₆alkoxy, or halogenated C₁₋₆alkoxy.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁸ is hydrogen, methoxy or hydroxy.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁸ is methoxy.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁸ is OH.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁸ is hydrogen.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof or a deuterated form thereof, R⁰ is 5-12 membered monocyclic heterocyclyl containing 1-3 heteroatoms selected from N, S, O, wherein the monocyclic heterocycle is optionally substituted with 1-3 R⁸ groups. In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

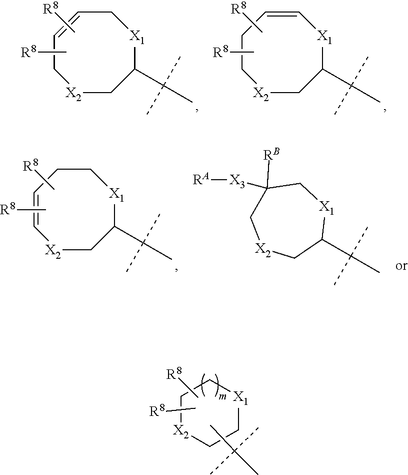

wherein
X₁ and X₂ are independently O, S, NH, N(C₁₋₆alkyl), or CR¹²R¹³, wherein at least one of X₁ and X₂ are not CR¹²R¹³;
X₃ is O, S, NH, or N(C₁₋₆alkyl);
R⁸ is H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy or halogenated C₁₋₆alkoxy;
R¹² is H, halogen, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy or halogenated C₁₋₆alkoxy
R¹³ is independently H, halogen or C₁-C₆ alkyl;
R^{A} is H, C₁₋₆alkyl, C₁₋₆alkylene-carbocyclyl, or C₁₋₆alkylene-heteroaryl; and
R^{B} is C₁₋₆alkyl, C₂₋₆alkenyl, C₁₋₆alkylene-carbocyclyl, or C₁₋₆alkylene-heteroaryl, or
R^{A} and R^{B} are taken together to form a heterocyclyl; and
m is 0, 1, 2 or 3.

In embodiments, of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R¹³ is independently H, F, Cl, Br, I or C₁-C₆ alkyl. In embodiments, R¹³ is independently H, F, or C₁-C₆ alkyl. In embodiments, R¹³ is H.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R¹² is H, OH, halogen, NH₂, COOH, unsubstituted C₁₋₄alkyl, C₁₋₄ alkyl-OH, unsubstituted C₁₋₄alkoxy or halogenated C₁₋₄alkoxy.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R¹² is hydrogen.

In embodiments of a compound of Formula (I), (II), (III), (IV), (V), (VI), (VII), (VIII), (IX), (X), (XI), (XII), (XIII), (XIV), (XV) or (XVI) or a pharmaceutically acceptable salt thereof or deuterated form thereof, X is O, S CHF, or CF₂.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof or deuterated form thereof, X is O, S or CF₂.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof or deuterated form thereof, X is O.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof or deuterated form thereof, X is S.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof or deuterated form thereof, X is CF₂.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, X₁ and X₂ are independently O, S, NH, N(C₁₋₆alkyl), or CR¹²R¹³, wherein at least one of X₁ and X₂ are not CR¹²R¹³. In embodiments, at least X₁ is O, S, NH, N(C₁₋₆alkyl).

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, X₁ and X₂ are independently O, S, or NH, N(C₁₋₆alkyl).

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, X₁ and X₂ are independently O or NH.

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, X₃ is O, S, NH, or N(C₁₋₆alkyl).

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, X₃ is O, S, NH.

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, X₃ is O.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

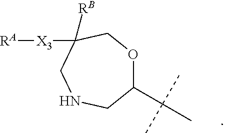

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R^{A} is H, C₁₋₆alkyl, C₁₋₆alkylene-carbocyclyl, or C₁₋₆alkylene-heteroaryl.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof,
R^{A} is H, C₁₋₆alkyl, C₁₋₆alkylene-carbocyclyl, or C₁₋₆alkylene-heteroaryl; and
R^{B} is C₁₋₆alkyl, C₂₋₆alkenyl, C₁₋₆alkylene-carbocyclyl, or C₁₋₆ alkylene-heteroaryl; or R^{A} and R^{B} are taken together to form a heterocyclyl.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R^{A} is H or C₁₋₆alkyl.

In an embodiment of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R^{A} is H.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R^{A} is C₁₋₆alkyl.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R^{A} is —CH₃.

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R^{B} is C₁₋₆alkyl, C₂₋₆alkenyl, C₁₋₆alkylene-carbocyclyl, or C₁₋₆alkylene-heteroaryl.

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R^{B} is C₁₋₆alkyl, C₂₋₆alkenyl, C₁₋₆alkylene-carbocyclyl, or C₁₋₆alkylene-heteroaryl.

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R^{B} is C₁₋₆alkyl, C₁₋₆alkylene-aryl or —C₁₋₆alkylene-5-6 membered heteroaryl.

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R^{B} is C₁₋₆alkyl.

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) R^{A} and R^{B} are taken together to form a heterocyclyl.

Another embodiment is a product obtainable by any of the processes or examples disclosed herein.

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

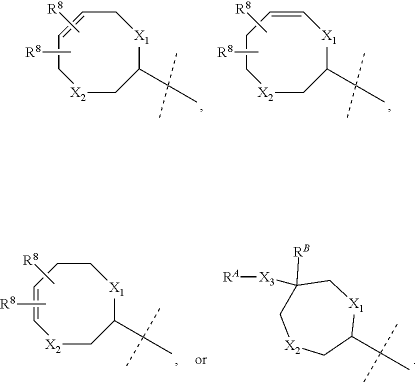

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

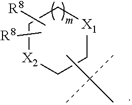

In embodiments, m is 2 or 3.

In embodiments, m is 2. In embodiments of the compounds, R⁰ is

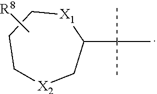

In embodiments of, R⁰ is

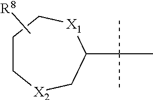

wherein X₁ is O, X₂ is NH, and R⁰ is C₁₋₆alkoxy.

In embodiments, R⁰ is

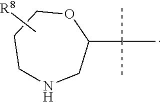

In embodiments, R⁰ is

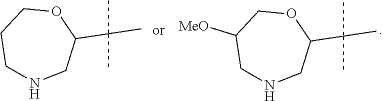

In embodiments, R⁰ is

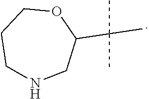

In embodiments, R⁰ is

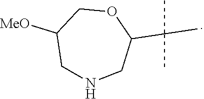

In one embodiment of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof or a deuterated form thereof, R⁰ is

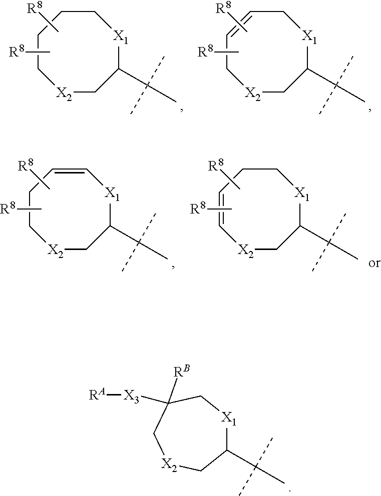

In embodiments, R⁰ is

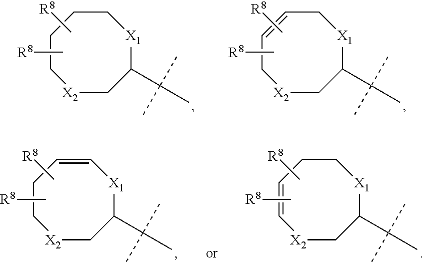

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof or a deuterated form thereof, R⁰ is

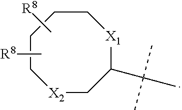

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof or a deuterated form thereof, R⁰ is

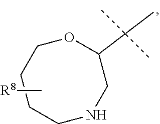

wherein each R⁸ is independently selected from H, halogen, C₁₋₆ alkyl, C₁₋₆alkoxy or halogenated C₁₋₆alkoxy.

In embodiments, R⁰ is:

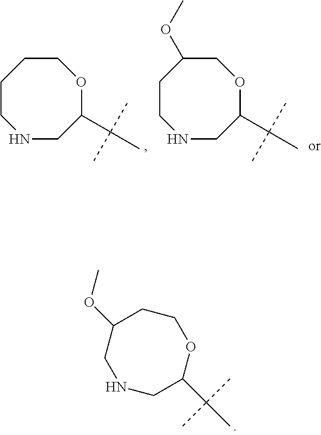

In embodiments, R⁰ is:

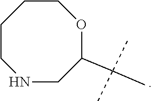

In embodiments, R⁰ is:

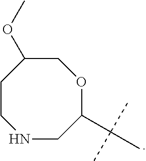

In embodiments, R⁰ is:

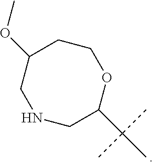

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof or a deuterated form thereof, R⁰ is

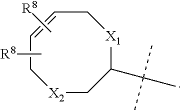

In embodiments, R⁰ is

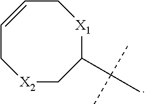

In embodiments, R⁰ is

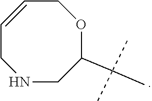

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof or a deuterated form thereof, R⁰ is

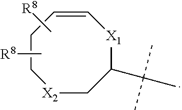

In embodiments, R⁰ is

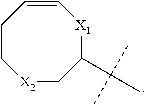

In embodiments, R⁰ is

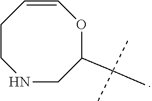

In embodiments of a compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

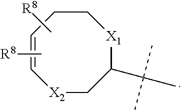

In embodiments, R⁰ is

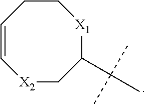

In embodiments, R⁰ is

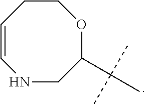

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

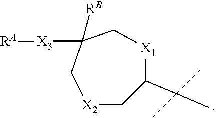

In embodiments, R⁰ is

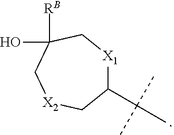

In embodiments, R⁰ is

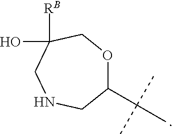

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

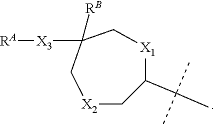

In embodiments, R⁰ is

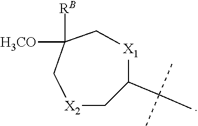

In embodiments, R⁰ is

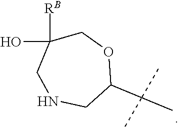

In embodiments, R⁰ is

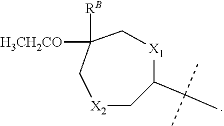

In embodiments, R⁰ is

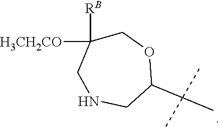

In embodiments of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is:

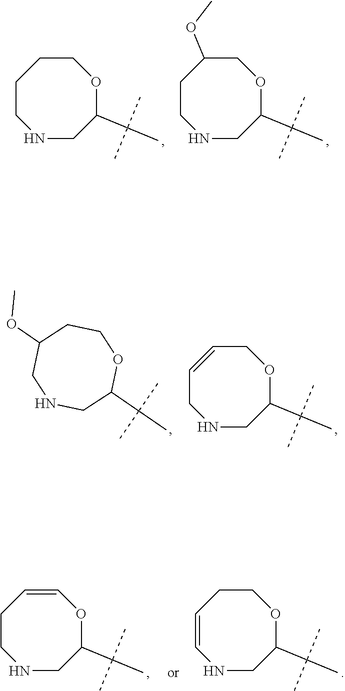

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

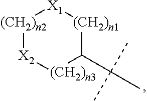

wherein each n1, n2 and n3 is independently an integer from 0-3, and the total sum of n1, n2 and n3 is ≤4, X₁ and X₂ are independently O, S, NR⁶ or CR¹²R¹³, wherein at least one of the X₁ and X₂ is not CR¹²R¹³; each R⁶, R¹² and R¹³ is independently H, halo, or C₁-C₆ alkyl. In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

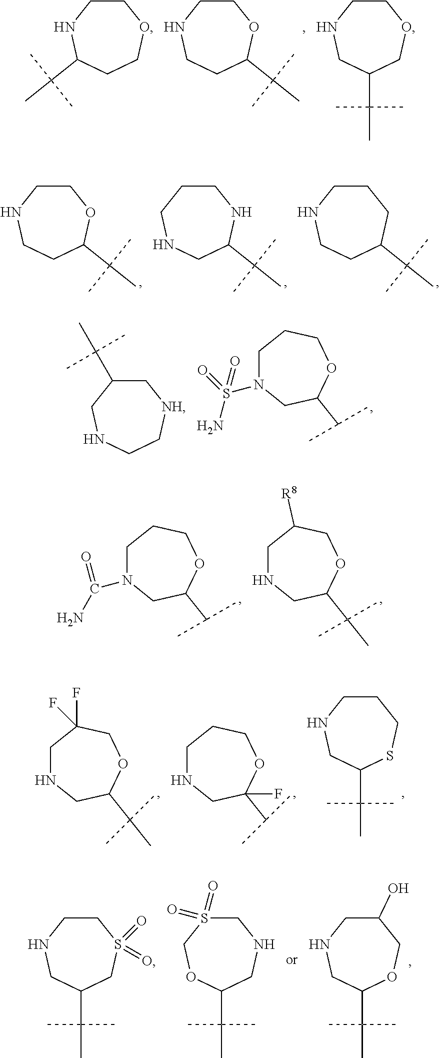

wherein R⁸ is H, F, OH, CH₃, OCH₃, OCHF₂, OCF₃, OCH₂CH₃, or —CH₂OCH₃.

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

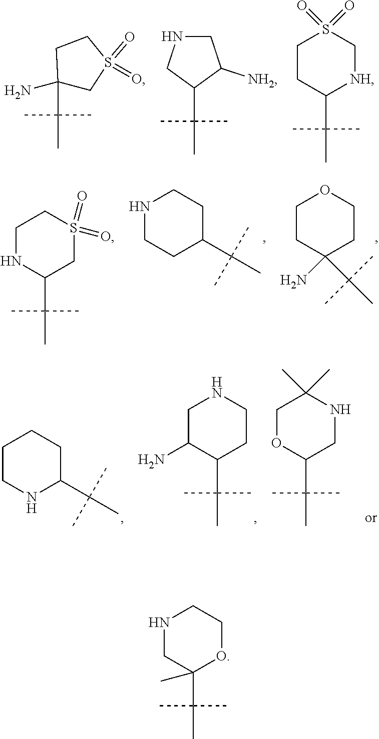

In embodiments, R⁰ is

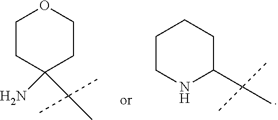

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is 5-12-membered polycyclic heterocycle containing 1-3 heteroatoms selected from N, S, O, wherein the polycyclic heterocycle is optionally substituted with 1-3 R⁸. In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, the 5-12-membered polycyclic heterocycle is
(i) 5-12 membered spiro heterocycle containing 1-3 heteroatoms selected from N, S, O, wherein the spiro heterocycle is optionally substituted with 1-3 R⁸,
(ii) 5-12 membered fused heterocycle containing 1-3 heteroatoms selected from N, S, O, wherein the fused heterocycle is optionally substituted with 1-3 R⁸, or
(iii) 7-12 membered bridged heterocycle containing 1-3 heteroatoms selected from N, S, O, wherein the bridge heterocycle is optionally substituted with 1-3 R⁸.

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is 5-12 membered spiro heterocycle containing 1-3 heteroatoms selected from N, S, O, wherein the spiro heterocycle is optionally substituted with 1-3 R⁸.

In embodiments, the 5-12 membered spiro heterocycle is

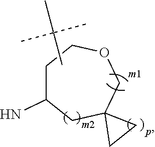

m1 and m2 are each independently 0, 1, or 2, provided that both m1 and m2 are not 0, or both m1 and m2 are not 2, and p is 1 or 2.

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is 5-12 membered fused heterocycle containing 1-3 heteroatoms selected from N, S, O, wherein the fused heterocycle is optionally substituted with 1-3 R⁸.

In embodiments, the 5-12 membered fused heterocycle is

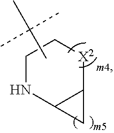

m4 is 0 or 1,
m5 is 1 or 2,
X² is O, S, NH, N(C₁₋₆alkyl), or CR¹²R¹³,
R¹² is selected from H, halogen, cyano, hydroxyl, NH₂, NH(C₁₋₆alkyl), N(C₁₋₆alkyl)₂, COOH, C₁₋₆ alkyl, —CONH₂, —S(═O)NH₂, —S(O)₂NH₂, C₁₋₆alkoxy or halogenated C₁₋₆alkoxy;
R¹³ is independently H, F, Cl, Br, I or C₁-C₆ alkyl.

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof,

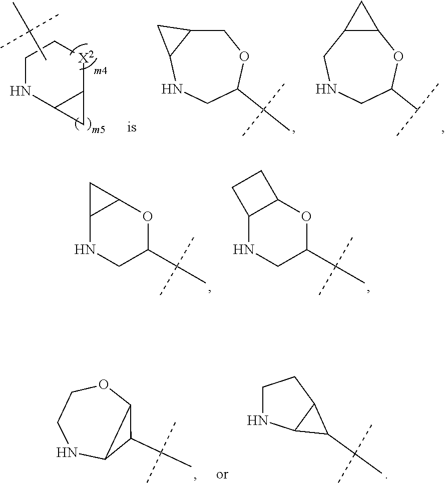

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

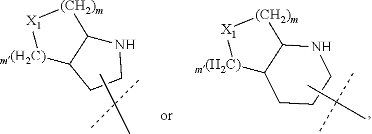

wherein
X₂ is O, S, NH, N(C₁₋₆alkyl), or CR¹²R¹³
R¹² is H, halogen, cyano, hydroxyl, NH₂, NH(C₁₋₆alkyl), N(C₁₋₆alkyl)₂, COOH, C₁₋₆ alkyl, —CONH₂, —S(═O)NH₂, —S(O)₂NH₂, C₁₋₆alkoxy or halogenated C₁₋₆alkoxy;
R¹³ is H, halogen or C₁-C₆ alkyl;
each m and m′ is independently an integer from 0-3, and the total sum of m and m′ is ≤3.

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

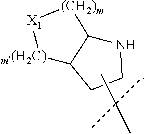

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

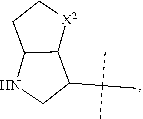

wherein X² is NH, O, or S.

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

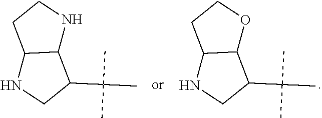

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

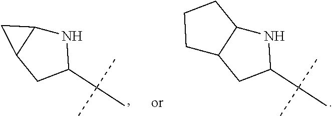

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

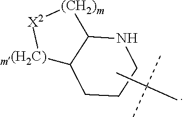

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

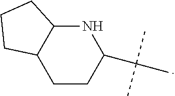

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is 7-12 membered bridged heterocycle containing 1-3 heteroatoms selected from N, S, O, wherein the bridged heterocycle is optionally substituted with 1-3 R⁸.

In embodiments, the 7-12 membered bridged heterocycle is

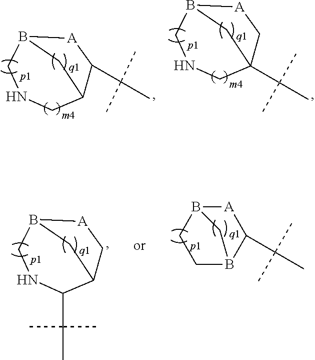

each of which is optionally substituted with 1-4 R⁸, wherein
A is a bond, —O—, —O—CH₂—, —CH₂—O—CH₂—, —CH₂OCH₂CH₂—, —CH₂—, —CH₂CH₂—, or —CH₂NH—
B is N or CH,
m4 is 0 or 1,
p1 is 0, 1, or 2,
q1 is 1, 2, or 3.

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is

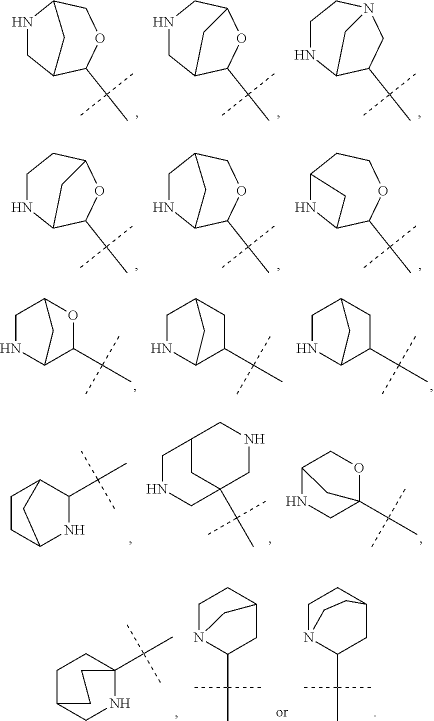

In embodiments of the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof, R⁰ is selected from

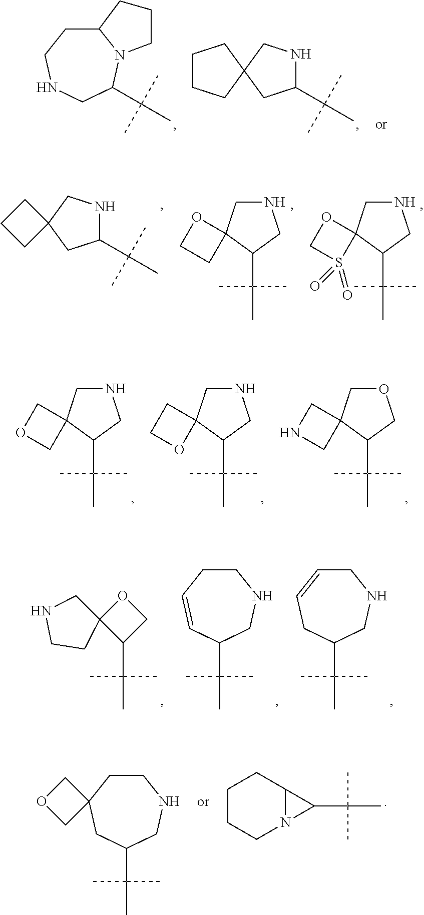

### L

In embodiments of the compounds of Formula (I), or a pharmaceutically acceptable salt thereof, or a deuterated form thereof, L is polycyclic cycloalkylene, polycyclic arylene or polycyclic heteroarylene.

In embodiments, the polycyclic cycloalkylene is connected to R¹ at a first quaternary carbon of the polycyclic cycloalkylene and independently connected to

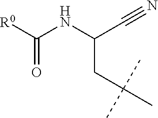

through a second quaternary carbon of the polycyclic cycloalkylene.

In embodiments, a first atom of a first ring of the polycyclic arylene or polycyclic heteroarylene is connected to

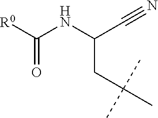

and a second atom of a second ring of the polycyclic arylene or heteroarylene is connected to R¹.

In embodiments, L is independently substituted by 0-4 R¹⁰.

In embodiments, each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH.

In embodiments, L is a polycyclic cycloalkylene.

In embodiments, the polycyclic cycloalkylene comprises 4-10 carbon atoms.

In embodiments, the polycyclic cycloalkylene is connected to R¹ at a first quaternary carbon of the polycyclic cycloalkylene and independently connected to

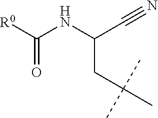

through a second quaternary carbon of the polycyclic cycloalkylene.

In embodiments of the compounds of Formula (I), or a pharmaceutically acceptable salt thereof, or a deuterated form thereof, L is

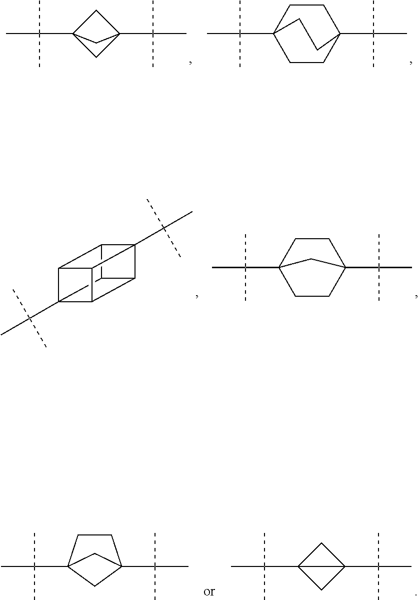

In further embodiments, L is

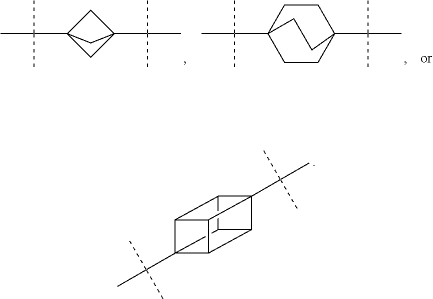

In embodiments, L is

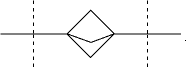

In embodiments, L is

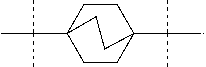

In embodiments, L is

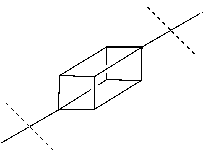

In embodiments of the compounds of Formula (I), or a pharmaceutically acceptable salt thereof, or a deuterated form thereof, L is polycyclic heteroarylene substituted by 0-4 R¹⁰. In embodiments of the compounds of Formula (I), or a pharmaceutically acceptable salt thereof, or a deuterated form thereof, L is a polycyclic heteroarylene comprising 8-12 ring atoms and is substituted by 0-4 R¹⁰. In embodiments of the compounds of Formula (I), or a pharmaceutically acceptable salt thereof, or a deuterated form thereof, L is a polycyclic heteroarylene comprising 9-10 ring atoms and is substituted by 0-4 R¹⁰. In embodiments of the compounds of Formula (I), or a pharmaceutically acceptable salt or deuterated form thereof, the polycyclic heteroarylene comprises 8-10 ring atoms and is substituted by 0-4 R¹⁰. In embodiments of the compounds of Formula (I), or a pharmaceutically acceptable salt or deuterated form thereof, the polycyclic heteroarylene comprises 8-9 ring atoms and is substituted by 0-4 R¹⁰. In embodiments of the compounds of Formula (I), or a pharmaceutically acceptable salt thereof, or a deuterated form thereof, L is a polycyclic heteroarylene comprises 9 ring atoms and 1 or 2 heteroatoms selected from O, N, or S, and the polycyclic heteroarylene is substituted by 0-4 R¹⁰. In embodiments of the compounds of Formula (I), or a pharmaceutically acceptable salt thereof, or a deuterated form thereof, L is a polycyclic heteroarylene comprises 8 ring atoms and 1-3 heteroatoms selected from O, N, or S, and the polycyclic heteroarylene is substituted by 0-4 R¹⁰. In embodiments of the compounds of Formula (I), or a pharmaceutically acceptable salt thereof, or a deuterated form thereof, L is polycyclic heteroarylene substituted by zero (0) R¹⁰. In embodiments of the compounds of Formula (I), or a pharmaceutically acceptable salt thereof, or a deuterated form thereof, L is polycyclic heteroarylene substituted by one (1) R¹⁰.

In embodiments of the compounds of Formula (I), or a pharmaceutically acceptable salt or deuterated form thereof, L is

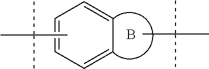

substituted by 0-4 R¹⁰, wherein ring B is a heteroaryl ring. When L is polycyclic heteroarylene, the polycyclic heteroarylene may have any orientation

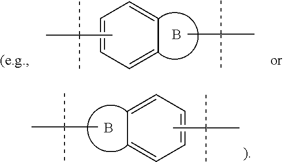

In embodiments of the compounds of Formula (I), or a pharmaceutically acceptable salt thereof, or a deuterated form thereof, the polycyclic heteroarylene is

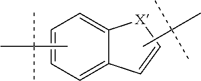

substituted by 0-4 R¹⁰, wherein X′ is O, S, NH or N(C₁₋₆ alkyl). In embodiments of the compounds of Formula (I), or a pharmaceutically acceptable salt thereof, or a deuterated form thereof, the polycyclic heteroarylene is benzothienylene, indolylene or benzofuranylene substituted by 0-4 R¹⁰.

In embodiments of the compounds of Formula (I), or a pharmaceutically acceptable salt thereof, or a deuterated form thereof, the polycyclic heteroarylene is

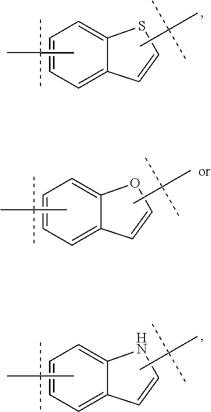

each of which is substituted by 0-4 R¹⁰.

In embodiments of the compounds of Formula (I), or a pharmaceutically acceptable salt thereof, or a deuterated form thereof, the polycyclic heteroarylene is

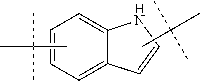

substituted by 0-4 R¹⁰.

In embodiments of the compounds of Formula (I), or a pharmaceutically acceptable salt thereof, or a deuterated form thereof, the polycyclic heteroarylene is

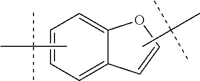

substituted by 0-4 R¹⁰.

In embodiments of the compounds of Formula (I), or a pharmaceutically acceptable salt thereof, or a deuterated form thereof, L is benzothienylene substituted by 0-4 R¹⁰.

In embodiments, L is

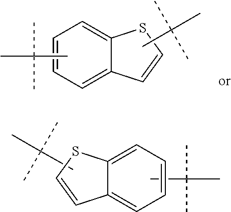

substituted by 0-4 R¹⁰.

In embodiments, L is

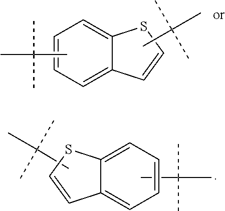

In embodiments of the compounds of Formula (I), or a pharmaceutically acceptable salt thereof, or a deuterated form thereof,

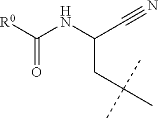

is attached to the phenyl ring of

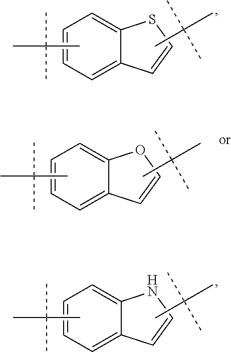

and R¹ is attached the 5-membered ring of

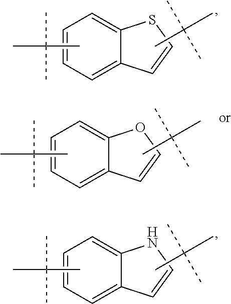

wherein

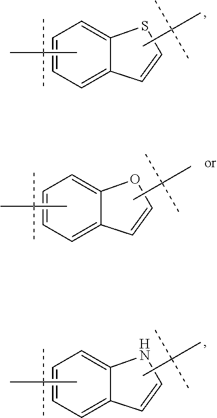

is substituted by 0-4 R¹⁰.

In embodiments of the compounds of Formula (I), or a pharmaceutically acceptable salt or deuterated form thereof, wherein

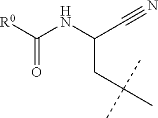

is attached to the 5-membered ring of

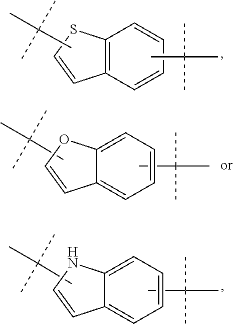

and R¹ is attached to the phenyl ring of

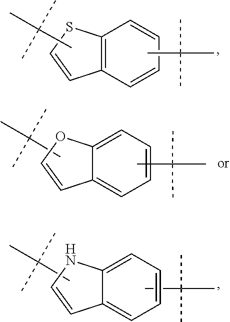

and wherein

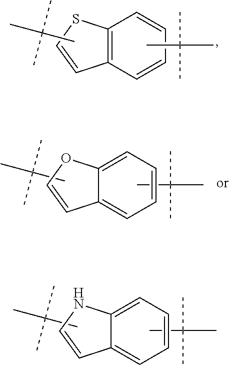

is substituted by 0-4 R¹⁰.

In embodiments of the compounds of Formula (I), or a pharmaceutically acceptable salt or deuterated form thereof, the polycyclic heteroarylene is

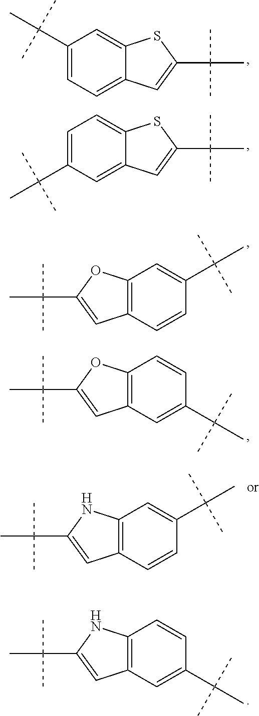

In embodiments of the compounds of Formula (I), or a pharmaceutically acceptable salt or deuterated form thereof, L is

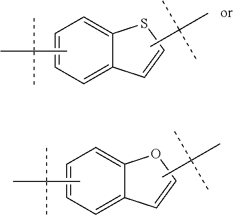

or substituted by 1-4 R¹⁰.

In embodiments of the compounds of Formula (I) or a pharmaceutically acceptable salt or deuterated form thereof, L is

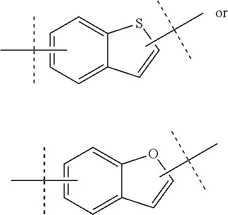

or substituted by one R¹⁰.

In embodiments of the compounds of Formula (I), or a pharmaceutically acceptable salt or deuterated form thereof, R¹⁰ is halo.

In embodiments of the compounds of Formula (I), or a pharmaceutically acceptable salt or deuterated form thereof, L is

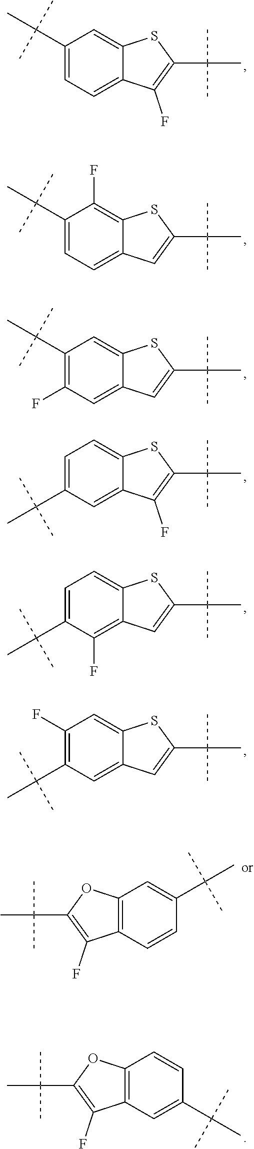

In embodiments of the compounds of Formula (I), or a pharmaceutically acceptable salt or deuterated form thereof, L is the polycyclic heteroarylene comprising 8 ring atoms and 1 or 2 heteroatoms selected from O, N, or S, and the polycyclic heteroarylene is substituted by 0-4 R¹⁰.

In embodiments of the compounds of Formula (I), or a pharmaceutically acceptable salt or deuterated form thereof, is L is

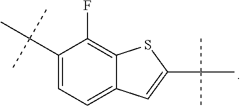

In embodiments of the compounds of Formula (I) or (XVI), or a pharmaceutically acceptable salt or deuterated form thereof, L is

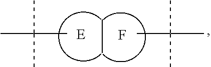

wherein each of ring E and ring F is 5-membered heteroaryl or heterocyclyl comprising 1-3 heteroatoms selected from O, N or S.

In embodiments of the compounds of Formula (I) or (XVI), or a pharmaceutically acceptable salt or deuterated form thereof, L is

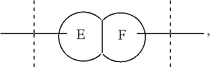

wherein each of ring E and ring F is 5-membered heteroaryl comprising 1-3 heteroatoms selected from O, N or S.

In embodiments of the compounds of Formula (I) or (XVI), or a pharmaceutically acceptable salt or deuterated form thereof, wherein L is

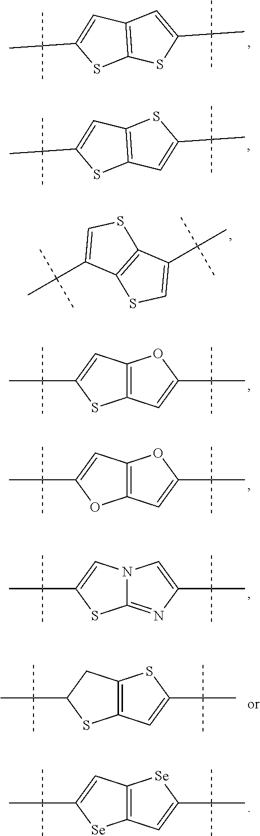

In embodiments of the compounds of Formula (I) or (XVI), or a pharmaceutically acceptable salt or deuterated form thereof, wherein L is

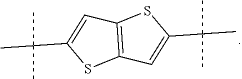

In embodiments of the compounds of Formula (I), wherein L is polycyclic arylene.

In embodiments of the compounds of Formula (I), wherein L is

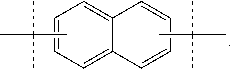

In embodiments of the compounds of Formula (I), wherein L is

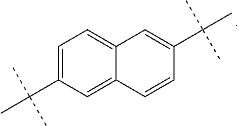

In embodiments of the compounds of Formula (I), (V), (VI), (VII), (XI), (XII), (XIII), or a pharmaceutically acceptable salt thereof, or a deuterated form thereof, R¹⁰ is independently oxo, halogen, C₁₋₆alkyl, C₁₋₆alkoxy, —S—C₁₋₆alkyl, C₂₋₆alkenyl, C₂₋₆ alkynyl, C₃₋₆cycloalkyl, cyano, hydroxy, NH₂, —NH—C₁₋₆alkyl, —N(C₁₋₄alkyl)₂, —COOH, —COC₁₋₆alkyl, —COOC₁₋₆ alkyl, —CON₁₋₆alkyl, —CON(C₁₋₆alkyl)₂, —NHCOC₁₋₆alkyl, or a heterocycle; wherein each alkyl, alkoxy, alkenyl, alkynyl, cycloalkyl, and heterocyclic are independently optionally substituted with 1-3 substituents selected from halogen, cyano, hydroxyl, NH₂ and —COOH.

In embodiments of the compounds of Formula (I), (V), (VI), (VII), (XI), (XII), (XIII), or a pharmaceutically acceptable salt thereof, or a deuterated form thereof, R¹⁰ is independently oxo, halogen, C₁₋₆alkyl, C₁₋₆alkoxy, —S—C₁₋₆alkyl, C₂₋₆alkenyl, C₂₋₆alkynyl, C₃₋₆cycloalkyl, cyano, hydroxy, NH₂, —NH—C₁₋₆alkyl, —N(C₁₋₄alkyl)₂, —COOH, —COC₁₋₆alkyl, —COOC₁₋₆alkyl, —CON₁₋₆alkyl, —CON(C₁₋₆alkyl)₂, —NHCOC₁₋₆alkyl, or 4-7 membered heterocycle containing 1-3 heteroatoms selected from N, S, and O; wherein each alkyl, alkoxy, alkenyl, alkynyl, cycloalkyl, and heterocycle are independently optionally substituted with 1-3 substituents selected from halogen, cyano, hydroxyl, NH₂ and —COOH.

In embodiments of the compounds of Formula (I), (V), (VI), (VII), (XI), (XII), (XIII), or a pharmaceutically acceptable salt thereof, or a deuterated form thereof, R¹⁰ is independently halogen, C₁₋₄alkyl, C₁₋₆alkoxy, —S—C₁₋₆alkyl, cyano, hydroxy, NH₂, —NH—C₁₋₆ alkyl, —N(C₁₋₆alkyl)₂, —COOH, —COC₁₋₆ alkyl, —COOC₁₋₆alkyl, —CON₁₋₆alkyl, —CON(C₁₋₆alkyl)₂, or —NHCOC₁₋₆alkyl; wherein each alkyl, and alkoxy are independently optionally substituted with 1-3 substituents selected from halogen, cyano, hydroxyl, NH₂ and —COOH.

In embodiments of the compounds of Formula (I), (V), (VI), (VII), (XI), (XII), (XIII), (XIV), (XV), (XVI), (XVII), (XVIII), (XIX), (XX) or a pharmaceutically acceptable salt thereof, or a deuterated form thereof, R¹⁰ is halogen.

In embodiments of the compounds of Formula (I), (V), (VI), (VII), (XI), (XII), (XIII), (XIV), (XV), (XVI), (XVII), (XVIII), (XIX), (XX), or a pharmaceutically acceptable salt thereof, or a deuterated form thereof, R¹⁰ is fluoro.

In embodiments of the compounds of Formula (I), (V), (VI), (VII), (XI), (XII), (XIII), (XIV), (XV), (XVI), (XVII), (XVIII), (XIX), (XX) or a pharmaceutically acceptable salt thereof, or a deuterated form thereof, n is 0.

In embodiments of the compounds of Formula (I), (V), (VI), (VII), (XI), (XII), (XIII), (XIV), (XV), (XVI), (XVII), (XVIII), (XIX), (XX) or a pharmaceutically acceptable salt thereof, or a deuterated form thereof, n is 1.

In embodiments of the compounds of Formula (I), (V), (VI), (VII), (XI), (XII), (XIII), (XIV), (XV), (XVI), (XVII), (XVIII), (XIX), (XX) or a pharmaceutically acceptable salt thereof, or a deuterated form thereof, n is 2.

In embodiments of the compounds of Formula (I), (V), (VI), (VII), (XI), (XII), (XIII), (XIV), (XV), (XVI), (XVII), (XVIII), (XIX), (XX) or a pharmaceutically acceptable salt thereof, or a deuterated form thereof, n is 1 and the R¹⁰ is fluoro.

In embodiments of the compounds of Formula (I), (V), (VI), (VII), (XI), (XII), (XIII), (XIV), (XV), (XVI), (XVII), (XVIII), (XIX), (XX) or a pharmaceutically acceptable salt thereof, or a deuterated form thereof, n is 1 and the R¹⁰ substituent is located on the phenyl ring of L, e.g.,

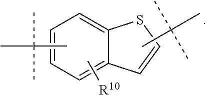

In embodiments of the compounds of Formula (I), (V), (VI), (VII), (XI), (XII), (XIII), or a pharmaceutically acceptable salt thereof, or a deuterated form thereof, n is 1 and the R¹⁰ substituent is located on the thiophene ring of L, e.g.,

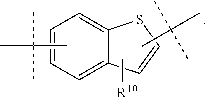

### R¹

In embodiments of the compounds of Formula (I), (II), (III), (IV), (V), (VI), (VII), (XIV), or a pharmaceutically acceptable salt thereof, or a deuterated form thereof, R¹ is (i) 5-12 membered carbocyclyl optionally substituted with 1-3 R^{g}; (ii) 6-18 membered aryl optionally substituted with 1-3 R^{g}; (iii) 5-12 membered monocyclic heterocyclyl containing 1-3 heteroatoms selected from N, S or O, wherein the monocyclic heterocyclyl is optionally substituted with 1-3 R^{g}; (iv) 5-12 membered monocyclic heteroaryl containing 1-3 heteroatoms selected from N, S or O, wherein the monocyclic heteroaryl is optionally substituted with 1-3 R^{g}; (v) 7-14 membered bicyclic heteroaryl containing 1-3 heteroatoms selected from N, S or O, wherein the bicyclic heteroaryl is optionally substituted with 1-4 R^{g}; or (vi) 7-20 membered tricyclic heteroaryl containing 1-3 heteroatoms selected from N, S or O, wherein the tricyclic heteroaryl is optionally substituted with 1-5 R^{g}. In embodiments, R¹ is 5-12 membered carbocyclyl optionally substituted with 1-3 R^{g}. In embodiments, R¹ is 6-18 membered aryl optionally substituted with 1-3 R^{g}. In embodiments, R¹ is 5-12 membered monocyclic heterocyclyl containing 1-3 heteroatoms selected from N, S or O, wherein the monocyclic heterocyclyl is optionally substituted with 1-3 R^{g}. In embodiments, R¹ is 5-12 membered monocyclic heteroaryl containing 1-3 heteroatoms selected from N, S or O, wherein the monocyclic heteroaryl is optionally substituted with 1-3 R^{g}. In embodiments, R¹ is 7-14 membered bicyclic heteroaryl containing 1-3 heteroatoms selected from N, S or O, wherein the bicyclic heteroaryl is optionally substituted with 1-4 R^{g}. In embodiments, R¹ is 7-20 membered tricyclic heteroaryl containing 1-3 heteroatoms selected from N, S or O, wherein the tricyclic heteroaryl is optionally substituted with 1-5 R^{g}. In embodiments, each R^{g} is independently selected from hydrogen, SF₅, ═O, halogen, cyano, hydroxyl, nitro, NH₂, —COOH, C₁₋₆ alkyl, C₁₋₆ alkoxy, C₃₋₆ cycloalkyloxy, C₂₋₆alkenyloxy, C₂₋₆ alkynyloxy, C₃₋₆ cycloalkyl, C₂₋₆ alkenyl, C₂₋₅ alkynyl, —NHC₁₋₆ alkyl, —N(C₁₋₆ alkyl)₂, —COC₁₋₆alkyl, —COC₁₋₆ alkyl, —CONH₂, —CONHC₁₋₆ alkyl, —CONHC₃₋₆ cycloalkyl, —CON(C₁₋₆ alkyl)₂, —NHCOC₁₋₆alkyl, —NHCOC₃₋₅cycloalkyl, —P(O)(C₁₋₆ alkyl), —S(O)C₁₋₆alkyl, —S(O)₂C₁₋₆ alkyl, —S(O)₂C₃₋₆cycloalkyl, —SO₂-3-7 membered heterocyclyl, —S(O)NH₂, —S(O)NHC₁₋₆ alkyl, —S(O)N(C₁₋₆ alkyl)₂, —S(O)₂NH₂, —OSO₂—C₁₋₆alkyl, C₁₋₆alkylene-O—C₁₋₆alkyl, C₃₋₈-cycloalkenyloxy, aryl, heteroaryl, 3-7-membered heterocycle containing 1-3 heteroatoms selected from N, S and O, and the R^{g} is optionally further substituted with 1-3 groups selected from C₁₋₆ alkyl, C₁₋₆ haloalkyl, C₁₋₆-alkylene-OH, —C(O)NH₂, NH₂, C₁₋₆ alkoxy, hydroxyl, —COOH, halogen, or a 5-7-membered heterocycle containing 1-3 heteroatoms selected from N, S and O, wherein the 5-7-membered heterocycle is optionally substituted with 1-2 groups selected from ═O, halogen, cyano, C₁₋₆ alkyl, and C₁₋₆ haloalkyl.

In embodiments of the compounds of Formula (I), (II), (III), (IV), (V), (VI), (VII), (XIV), or a pharmaceutically acceptable salt thereof, or a deuterated form thereof, R^{g} is selected from R⁶, R⁷, R¹¹, R¹⁴ or R¹⁵, wherein
each R⁶ and R¹¹ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH;
R¹⁴ and R¹⁵ are each independently selected from H, halogen, C₁₋₆ alkyl, C₁₋₆alkoxy, C₂₋₆ alkenyl, C₂₋₆ alkynyl, C₂₋₆alkenyloxy, C₂₋₆ alkynyloxy, C₃₋₆ cycloalkyl, C₃₋₆ cycloalkoxy, cyano, hydroxyl, NH₂, COOH, —S(O)C₁₋₆alkyl, —S(O)₂C₁₋₆ alkyl, —S(O)₂C₃₋₆cycloalkyl, —SO₂-3-7 membered heterocyclyl and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups selected from halogen, cyano, hydroxyl, NH₂ and —COOH;
alternatively, R¹⁴ and R¹⁵ form ═O.

In embodiments of the compounds of Formula (I), (II), (III), (IV), (V), (VI), (VII), (XIV), or a pharmaceutically acceptable salt thereof, or a deuterated form thereof, R¹ is 5-12 membered carbocyclyl optionally substituted with 1-3 R^{g} group. In embodiments, R¹ is

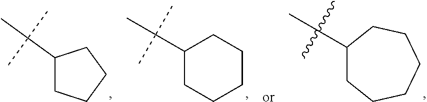

each of which is optionally substituted with 1-3 R^{g}.

In embodiments of the compounds of Formula (I), (II), (III), (IV), (V), (VI), (VII), (XIV), or a pharmaceutically acceptable salt thereof, or a deuterated form thereof, R¹ is 6-18 membered aryl optionally substituted with 1-3 R^{g} groups.

In embodiments, R¹ is

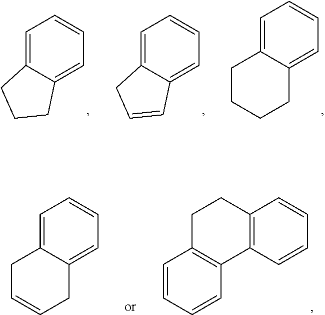

wherein L is attached to R¹ by replacing any hydrogen atom of R¹, and wherein each R¹ is optionally substituted with 1-3 R^{g}.

In embodiments, R¹ is

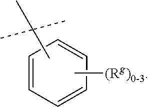

In embodiments, R¹ is

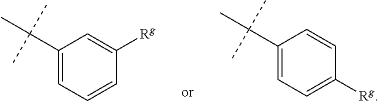

In embodiments, R¹ is

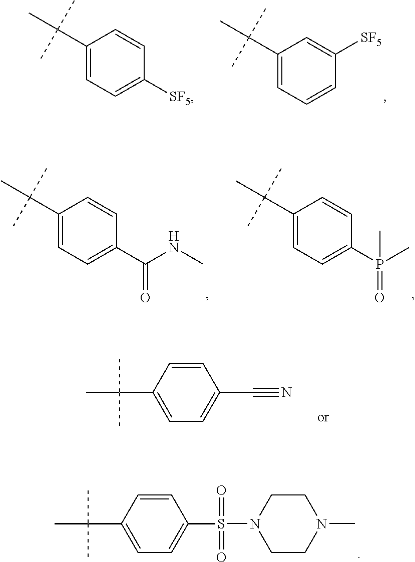

In embodiments, R¹ is

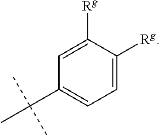

In embodiments, R¹ is

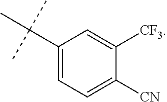

In embodiments of the compounds of formula (I), (II), (III), (IV), (V), (VI), (VII), (XIV), or a pharmaceutically acceptable salt thereof, or a deuterated form thereof, R¹ is 5-12 membered monocyclic heterocyclyl containing heteroatoms selected from N, S or O, wherein the monocyclic heterocyclyl is optionally substituted with 1-3 R^{g} group. In embodiments, R¹ is

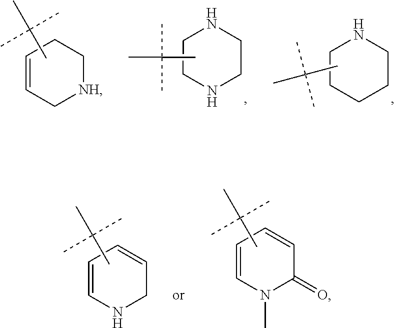

wherein each one of R¹ is optionally substituted with 1-3 R^{g}.

In embodiments of the compounds of Formula (I), (II), (III), (IV), (V), (VI), (VII), (XIV), or a pharmaceutically acceptable salt thereof, or a deuterated form thereof, R¹ is 5-12 membered monocyclic heteroaryl containing heteroatoms selected from N, S or O, wherein each one of the monocyclic heteroaryl is optionally substituted with 1-3 R^{g} group. In embodiments, R¹ is

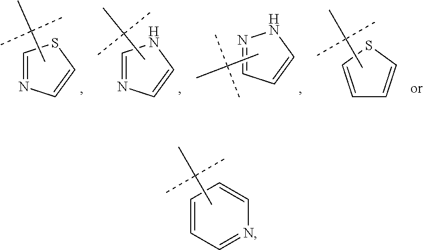

each of which is optionally substituted with 1-3 R^{g} group. In embodiments, R^{g} is independently H, halogen, C₁-C₆ alkyl, OSO₂C₁₋₆alkyl, or CN. In embodiments, R¹ is

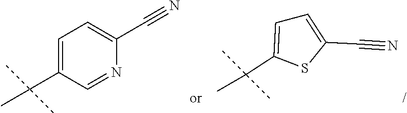

In embodiments, R¹ is

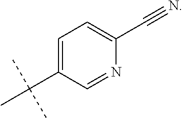

In embodiments, R¹ is

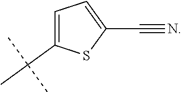

In embodiments of the compounds of Formula (I), (II), (III), (IV), (V), (VI), (VII), (XIV), or a pharmaceutically acceptable salt thereof, or a deuterated form thereof, R¹ is 7-14 membered bicyclic heteroaryl containing heteroatoms selected from N, S or O, wherein the bicyclic heteroaryl is optionally substituted with 1-4 R^{g} group.

In embodiments, R¹ is

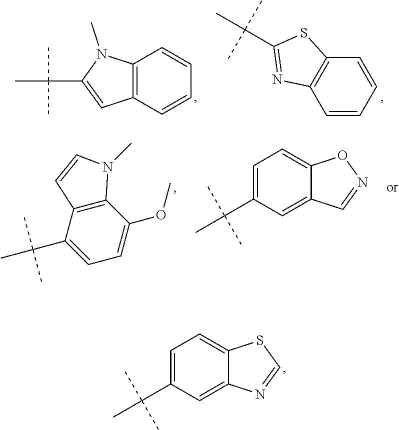

each of which is optionally substituted with 1-3 R^{g} group. In embodiments, R¹ is

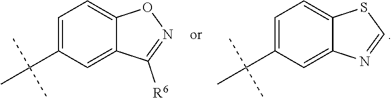

In embodiments, R¹ is

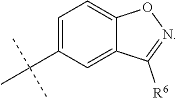

In embodiments of the compounds of Formula (I), (II), (III), (IV), (V), (VI), (VII), (XIV), or a pharmaceutically acceptable salt thereof, or a deuterated form thereof, R¹ is

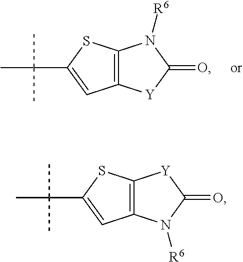

wherein
Y is independently O, S, CHR⁶ or NR⁶; and
R⁶ is C₁₋₆alkyl optionally substituted by 1, 2 or 3 F, or by OH, O—C₁₋₆alkyl, N(C₁₋₆alkyl)₂, cycloalkyl, or heterocyclyl.

In embodiments, R¹ is

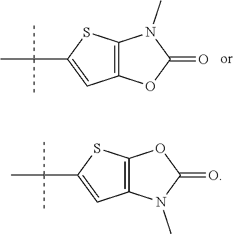

In embodiments of the compounds of Formula (I), (II), (III), (IV), (V), (VI), (VII), (XIV), or a pharmaceutically acceptable salt thereof, or a deuterated form thereof, R¹ is

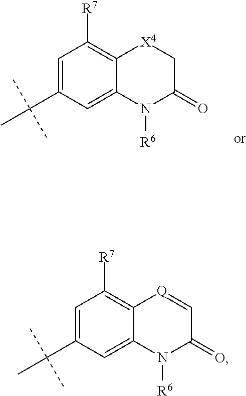

wherein
X⁴ is NR, O, CR⁷, CR¹⁴R¹⁵, S, S(O) or S(O)₂,
Q is CH or N,
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH;
R¹⁴ and R¹¹ are each independently selected from H, halogen, C₁₋₆ alkyl, C₁₋₆alkoxy, C₂₋₆ alkenyl, C₂₋₆ alkynyl, C₂₋₆alkenyloxy, C₂₋₆ alkynyloxy, C₃₋₆ cycloalkyl, C₃₋₆ cycloalkoxy, cyano, hydroxyl, NH₂, COOH, —S(O)C₁₋₆alkyl, —S(O)₂C₁₋₆ alkyl, —S(O)₂C₃₋₆cycloalkyl, —SO₂-3-7 membered heterocyclyl, and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups selected from halogen, cyano, hydroxyl, NH₂ and —COOH;
alternatively, R¹⁴ and R¹⁵ form ═O.

In embodiments, R¹ is

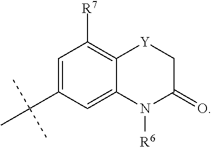

In embodiments, R¹ is

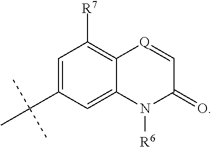

In embodiments of the compounds of Formula (I), (II), (III), (IV), (V), (VI), (VII), (XIV), or a pharmaceutically acceptable salt thereof, or a deuterated form thereof, R¹ is

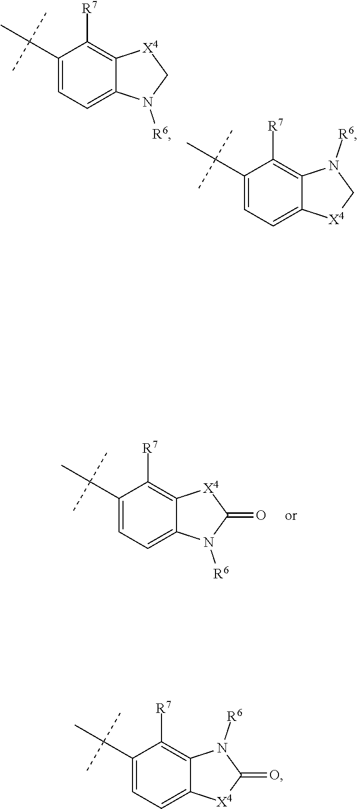

wherein
each X⁴ is independently NR⁶, O, CR¹⁴R¹⁵, S, S(O) or S(O)₂,
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH;
R¹⁴ and R¹¹ are each independently selected from H, halogen, C₁₋₆ alkyl, C₁₋₆alkoxy, C₂₋₆ alkenyl, C₂₋₆ alkynyl, C₂₋₆alkenyloxy, C₂₋₆ alkynyloxy, C₃₋₆ cycloalkyl, C₃₋₆ cycloalkoxy, cyano, hydroxyl, NH₂, COOH, —S(O)C₁₋₆alkyl, —S(O)₂C₁₋₆ alkyl, —S(O)₂C₃₋₆cycloalkyl, —SO₂-3-7 membered heterocyclyl, and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups selected from halogen, cyano, hydroxyl, NH₂ and —COOH; alternatively, R¹⁴ and R¹¹ form ═O.

In embodiments, is R¹ is

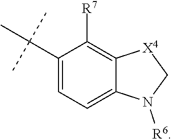

In some embodiments, X⁴ is independently 0, S, NR⁶ or CR¹⁴R¹⁵.

In embodiments, R⁶ is C₁₋₆alkyl, wherein the C₁₋₆alkyl is optionally substituted by 1, 2 or 3 F, OH, OC₁₋₆alkyl, N(C₁₋₆alkyl)₂, cycloalkyl, or heterocyclyl.

In embodiments, cycloalkyl is cyclopropyl.

In embodiments, heterocyclyl is tetrahydropyran. In embodiments R⁶ is C₁₋₆alkyl wherein said C₁₋₆alkyl is optionally substituted by 1, 2 or 3 F.

In embodiments, R⁶ is methyl or ethyl.

In embodiments, R⁶ is CH₃.

In embodiments, R⁷ is H, F, C₁ or CH₃.

In embodiments R⁷ is H.

In embodiments, R⁷ is H, halo or C₁₋₆alkyl, R¹⁴ and R¹⁵ are independently H, halo or C₁₋₆alkyl.

In embodiments, each X⁴ is independently NH, O, S, CHF, or CHF₂.

In embodiments, X⁴ is O.

In embodiments, R¹ is

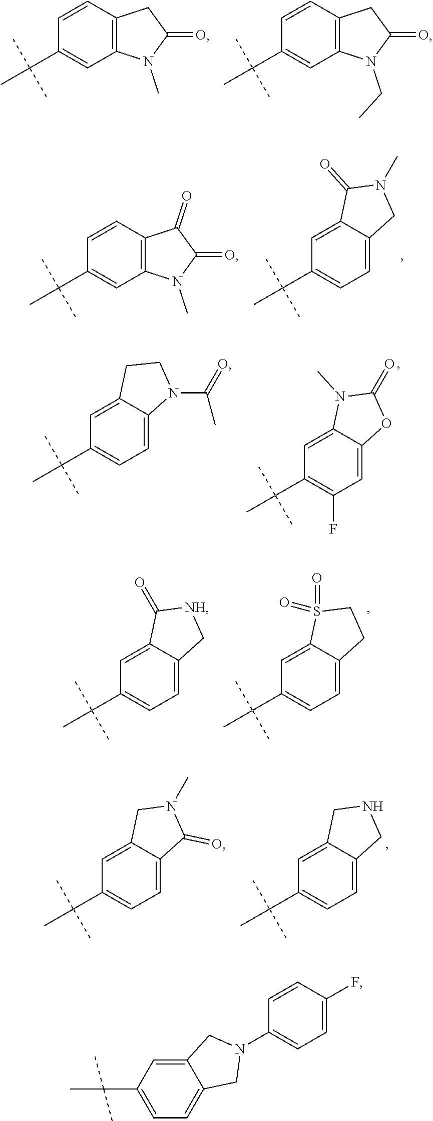

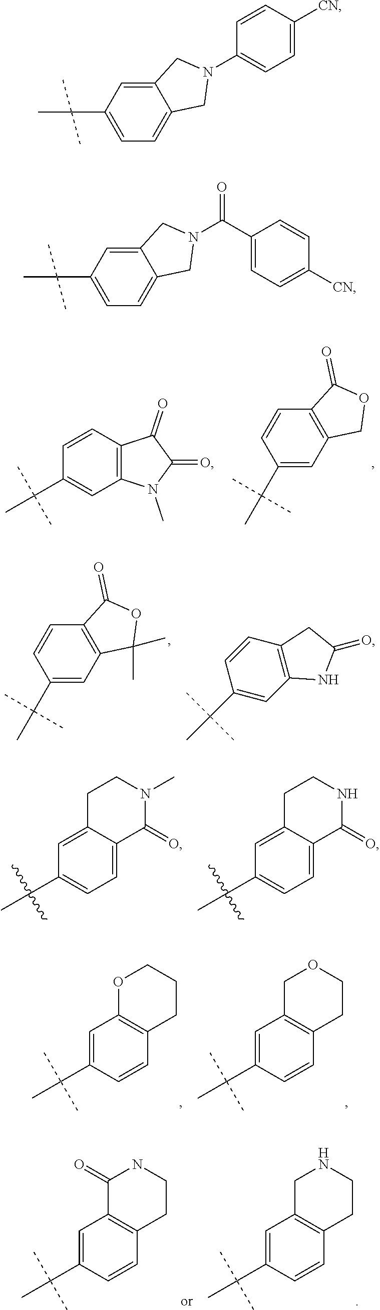

In embodiments, R¹ is

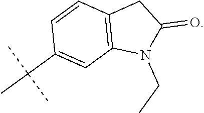

In embodiments, R¹ is

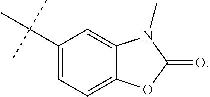

In embodiments, R¹ is

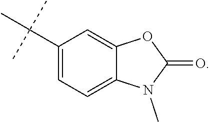

In embodiments of the compounds of Formula (I), (II), (III), (IV), (V), (VI), (VII), (XIV), or a pharmaceutically acceptable salt thereof, or a deuterated form thereof, R¹ is

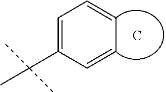

optionally substituted with 1-4 R^{g} group, wherein ring C is a 7-8 membered heterocycle comprising 1-3 heteroatoms selected from O, S and N.

In embodiments, R¹ is

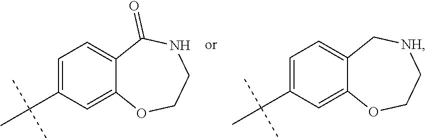

each of which is optionally substituted with 1-4 R^{g} groups.

In embodiments, R¹ is

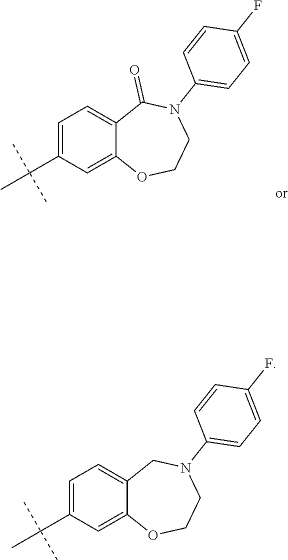

In embodiments of the compounds of Formula (I), (II), (III), (IV), (V), (VI), (VII), (XIV), or a pharmaceutically acceptable salt thereof, or a deuterated form thereof, R¹ is 7-20 membered tricyclic heteroaryl containing heteroatoms selected from N, S or O, wherein the tricyclic heteroaryl is optionally substituted with 1-5 R^{g}.

In embodiments of the compounds of Formula (I), (II), (III), (IV), (V), (VI), (VII), (XIV), or a pharmaceutically acceptable salt thereof, or a deuterated form thereof, R¹ is 7-20 membered spiro tricyclic heteroaryl containing heteroatoms selected from N, S or O, wherein the tricyclic heteroaryl is optionally substituted with 1-5 R^{g}.

In embodiments of the compounds of Formula (I), (II), (III), (IV), (V), (VI), (VII), (XIV), or a pharmaceutically acceptable salt thereof, or a deuterated form thereof, R¹ is

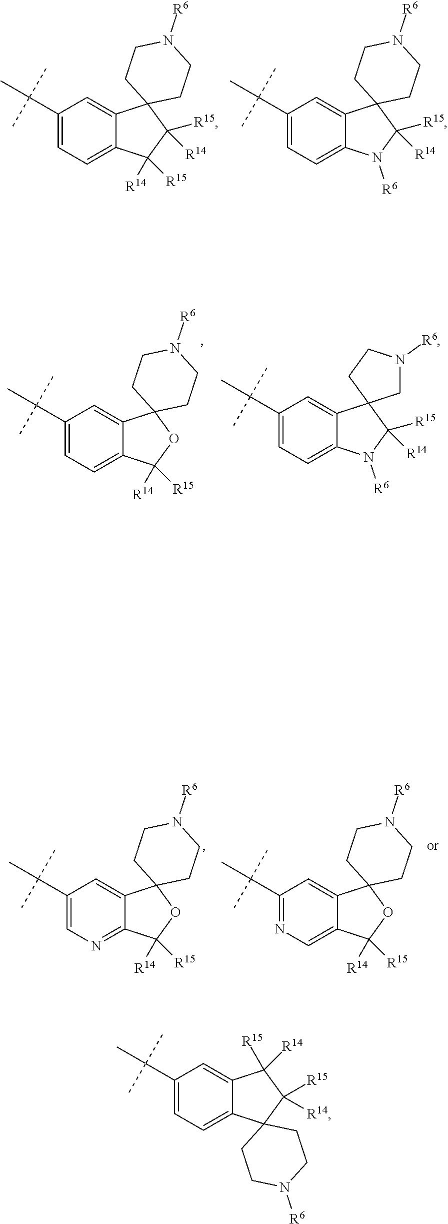

wherein
each R⁶ is independently selected from H, C₁₋₆ alkyl, —COC₁₋₆ alkyl, C₂₋₆ alkenyl, C₂₋₆alkynyl, C₃₋₆cycloalkyl or alkylene-O-alkyl; wherein the alkyl, alkenyl, alkynyl and cycloalkyl is optionally substituted with 1-3 groups selected from halogen, cyano, hydroxyl, NH₂ and COOH; and,
R¹⁴ and R¹⁵ are each independently selected from H, halogen, C₁₋₆ alkyl, C₁₋₆alkoxy, C₂₋₆ alkenyl, C₂₋₆ alkynyl, C₂₋₆alkenyloxy, C₂₋₆ alkynyloxy, C₃₋₆ cycloalkyl, C₃₋₆ cycloalkoxy, cyano, hydroxyl, NH₂, COOH, —S(O)C₁₋₆alkyl, —S(O)₂C₁₋₆ alkyl, —S(O)₂C₃₋₆cycloalkyl, —SO₂-3-7 membered heterocyclyl and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups selected from halogen, cyano, hydroxyl, NH₂ and —COOH;
alternatively, R¹⁴ and R¹⁵ form ═O.

In embodiments of the compounds of Formula (I), (II), (III), (IV), (V), (VI), (VII), (XIV), or a pharmaceutically acceptable salt thereof, or a deuterated form thereof, R¹ is

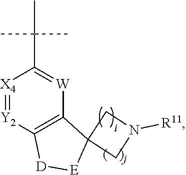

wherein
W, X₄ and Y₂ are each independently CH or N, provided that a maximum of one of W, X₄ and Y₂ can be N;
D-E is N(H)—C(═O), N(C₁₋₆alkyl)-C(═O), CH₂CH₂, C(═O)—O or CH₂—O;
R¹¹ is H, C₁₋₆alkyl, alkylene-O-alkyl, or heterocyclyl; and
i and j are each independently 1, 2 or 3; provided that the sum of i+j is 2, 3 or 4.

In embodiments of the compounds of Formula (I), (II), (III), (IV), (V), (VI), (VII), (VIII), (IX), (X), (XI), (XII), (XIII), (XIV), (XV) or (XVI), or a pharmaceutically acceptable salt thereof, or a deuterated form thereof, W, X₄ and Y₂ are each independently selected from CH and N, provided that that a maximum of one of W, X₄ and Y₂ can be N.

In embodiments of the compounds of Formula (I), (II), (III), (IV), (V), (VI), (VII), (VIII), (IX), (X), (XI), (XII), (XIII), (XIV), (XV) or (XVI), or a pharmaceutically acceptable salt thereof, or a deuterated form thereof, D-E is selected from N(H)—C(O), N(C₁₋₃-alkyl)-C(O), CH₂CH₂, C(O)—O and CH₂—O.

In embodiments of the compounds of Formula (I), (II), (III), (IV), (V), (VI), (VII), (VIII), (IX), (X), (XI), (XII), (XIII), (XIV), (XV) or (XVI), or a pharmaceutically acceptable salt thereof, or a deuterated form thereof, D-E is N(H)—C(O), N(CH₃)—C(O), CH₂CH₂, C(O)—O or CH₂—O.

In embodiments of the compounds of Formula (I), (II), (III), (IV), (V), (VI), (VII), (VIII), (IX), (X), (XI), (XII), (XIII), (XIV), (XV) or (XVI), or a pharmaceutically acceptable salt thereof, or a deuterated form thereof, D-E is CH₂—O;

In embodiments of the compounds of Formula (I), (II), (III), (IV), (V), (VI), (VII), (VIII), (IX), (X), (XI), (XII), (XIII), (XIV), (XV) or (XVI), or a pharmaceutically acceptable salt thereof, or a deuterated form thereof, R¹¹ is H, alkylene-O-alkyl, or heterocyclyl.

In embodiments of the compounds of Formula (I), (II), (III), (IV), (V), (VI), (VII), (VIII), (IX), (X), (XI), (XII), (XIII), (XIV), (XV) or (XVI), or a pharmaceutically acceptable salt thereof, or a deuterated form thereof, R¹¹ is H, CH₃OCH₂CH₂, or heterocyclyl.

In embodiments of the compounds of Formula (I), (II), (III), (IV), (V), (VI), (VII), (VIII), (IX), (X), (XI), (XII), (XIII), (XIV), (XV) or (XVI), or a pharmaceutically acceptable salt thereof, or a deuterated form thereof, R¹¹ is H, alkylene-O-alkyl, or oxetanyl, tetrahydrofuranyl, 4-tetrahydropyranyl or 3-tetrahydropyranyl.

In embodiments of the compounds of Formula (I), (II), (III), (IV), (V), (VI), (VII), (VIII), (IX), (X), (XI), (XII), (XIII), (XIV), (XV) or (XVI), or a pharmaceutically acceptable salt thereof, or a deuterated form thereof, R¹¹ is H, C₁₋₃-alkyl, CH₃OCH₂CH₂, oxetanyl, tetrahydrofuranyl, 4-tetrahydropyranyl or 3-tetrahydropyranyl.

In embodiments of the compounds of Formula (I), (II), (III), (IV), (V), (VI), (VII), (VIII), (IX), (X), (XI), (XII), (XIII), (XIV), (XV) or (XVI), or a pharmaceutically acceptable salt thereof, or a deuterated form thereof, R¹¹ is H, CH₃— or oxetanyl.

In embodiments of the compounds of Formula (I), (II), (III), (IV), (V), (VI), (VII), (VIII), (IX), (X), (XI), (XII), (XIII), (XIV), (XV) or (XVI), or a pharmaceutically acceptable salt thereof, or a deuterated form thereof, i and j are each independently 1, 2 or 3; provided that the sum of i+j is 2, 3 or 4.

In embodiments of the compounds of Formula (I), (II), (III), (IV), (V), (VI), (VII), (VIII), (IX), (X), (XI), (XII), (XIII), (XIV), (XV) or (XVI), or a pharmaceutically acceptable salt thereof, or a deuterated form thereof, R¹¹ is H, CH₃ or oxetanyl; W is CH or N; X₄ is CH or N; Y₂ is CH; provided that a maximum of one of W, X and Y can be N; D-E is selected from N(CH₃)—C(O), CH₂CH₂, C(O)—O and CH₂—O; i is 1 or 2 and j is 1 or 2 provided that the sum of i+j is 2, 3 or 4.

In embodiments of the compounds of Formula (I), (II), (III), (IV), (V), (VI), (VII), (XIV), or a pharmaceutically acceptable salt thereof, or a deuterated form thereof, R¹ is

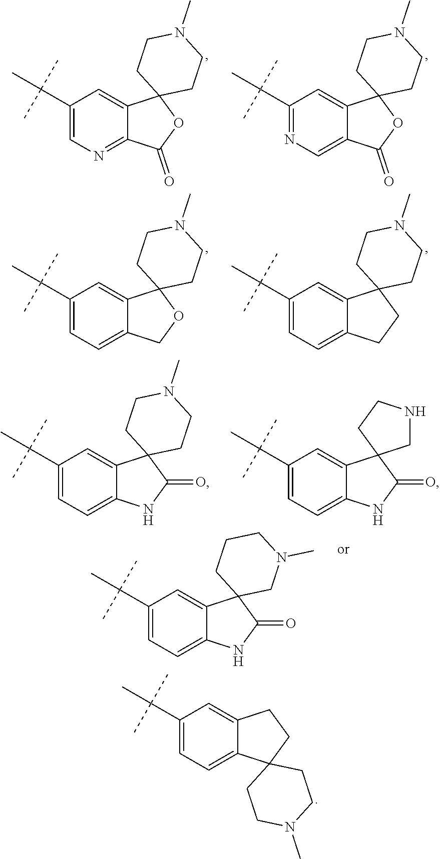

In embodiments, R¹ is

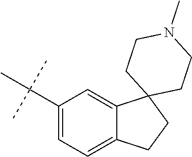

In embodiments of the compounds of Formula (I), (II), (III), (IV), (V), (VI), (VII), (XIV), or a pharmaceutically acceptable salt thereof, or a deuterated form thereof, R¹ is 7-20 membered fused tricyclic heteroaryl containing heteroatoms selected from N, S or O, wherein the tricyclic heteroaryl is optionally substituted with 1-5 R^{g}.

In embodiments of the compounds of Formula (I), (II), (III), (IV), (V), (VI), (VII), (XIV), or a pharmaceutically acceptable salt thereof, or a deuterated form thereof, R¹ is

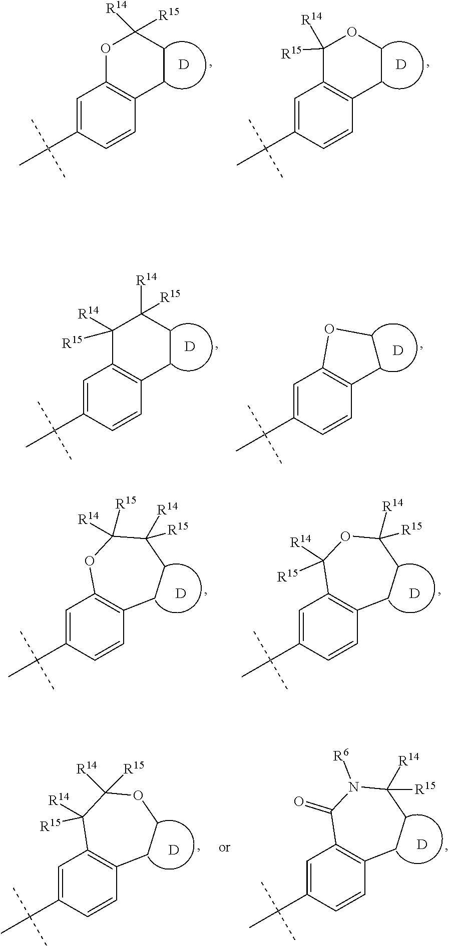

or each of which is optionally substituted with 1-5 R^{g}, wherein R¹⁴ and R¹⁵ are each independently selected from H, halogen, C₁₋₆ alkyl, C₁₋₆alkoxy, C₂₋₆ alkenyl, C₂₋₆ alkynyl, C₂₋₆alkenyloxy, C₂₋₆ alkynyloxy, C₃₋₆ cycloalkyl, C₃₋₆ cycloalkoxy, cyano, hydroxyl, NH₂, COOH, —S(O)C₁₋₆alkyl, —S(O)₂C₁₋₆ alkyl, —S(O)₂C₃₋₆cycloalkyl, —SO₂₋₃-7 membered heterocyclyl and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups selected from halogen, cyano, hydroxyl, NH₂ and —COOH;
each R⁶ is independently selected from H, C₁₋₆ alkyl, —COC₁₋₆ alkyl, C₂₋₆ alkenyl, C₂₋₆alkynyl, C₃₋₆cycloalkyl or alkylene-O-alkyl; wherein the alkyl, alkenyl, alkynyl and cycloalkyl is optionally substituted with 1-3 groups selected from halogen, cyano, hydroxyl, NH₂ and COOH;
Ring D is selected from aryl and 5-8 membered heteroaryl containing 1-3 heteroatoms, wherein aryl and said 5-8 membered heteroaryl containing 1-3 heteroatoms each independently optionally substituted with 1-3 R^{g}. In embodiments, Ring D is

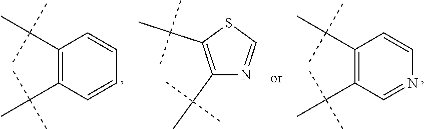

each of which is optionally substituted with 1-3 R^{g} group. In embodiments, R¹ is

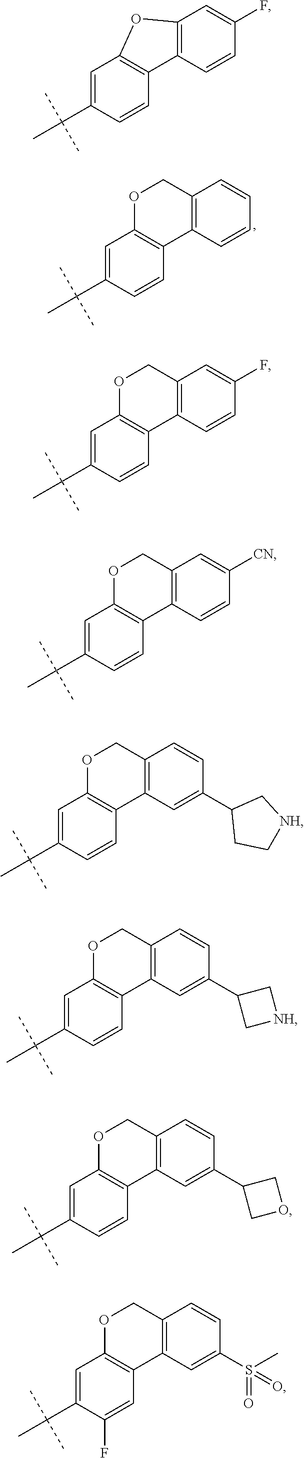

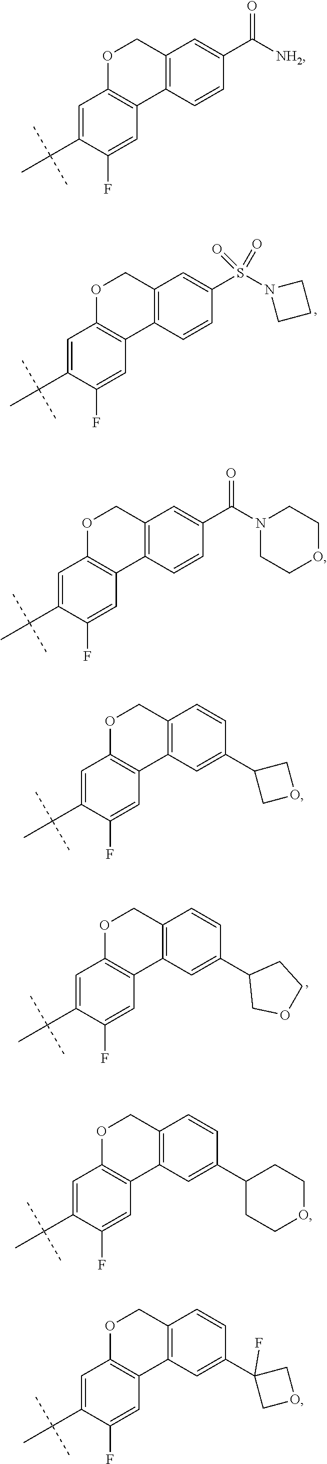

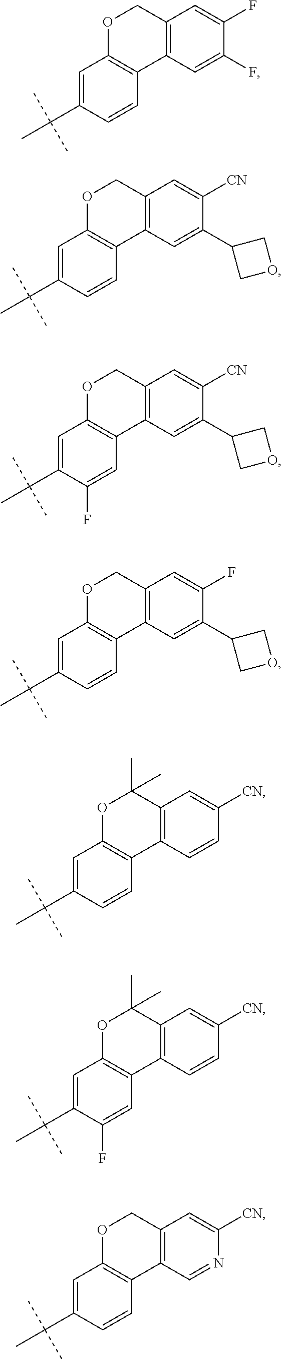

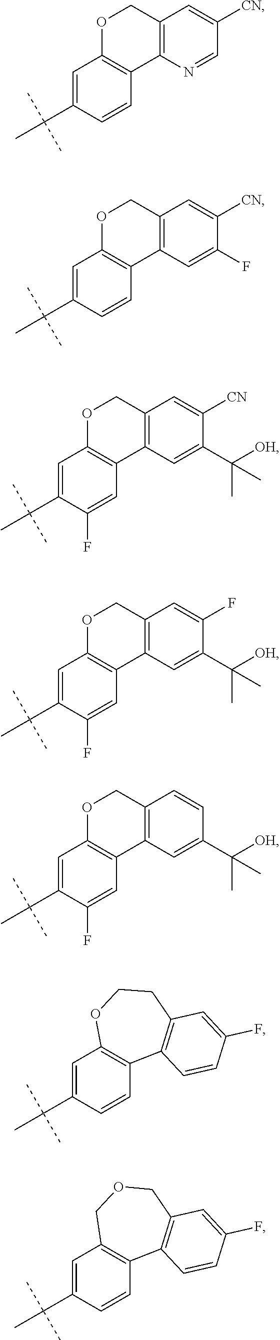

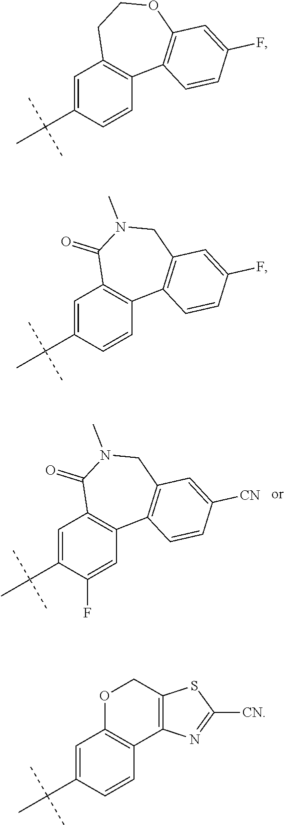

In embodiments, provided herein is a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX) or a stereoisomer thereof, or a pharmaceutically acceptable salt thereof, or a deuterated form thereof.

In embodiments, provided herein is a pharmaceutically acceptable salt of a compound (XV), (XVI), (XVII), (XVIII), (XIX) or (XX). Further embodiments of the invention relate to a deuterated compound of (I), (II), (III), (IV), (V), (VI), (VII), (VIII), (IX), (X), (XI), (XII), (XIII), (XIV), (XV), (XVI), (XVII), (XVIII), (XIX) or (XX) or a pharmaceutically acceptable salt thereof, or a deuterated form thereof.

In embodiments, provided herein is a compound in Table 1, or a pharmaceutically acceptable salt thereof, or a deuterated form thereof, or stereoisomer thereof.

TABLE 1

| Compound | |
|---|---|
| No. | Compound structure |
| 101 | 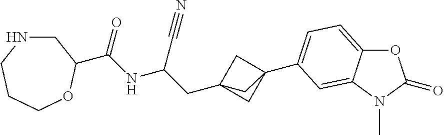 [SMILES: Cn1c(=O)oc2ccc(C34CC(CC(C#N)NC(=O)C5CNCCCO5)(C3)C4)cc21] |
| 101a | 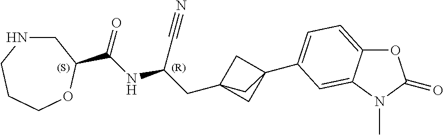 |
| 101b | 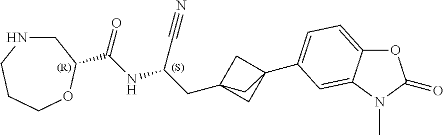 |
| 101c | 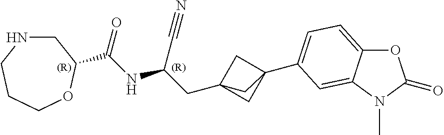 |
| 101d | 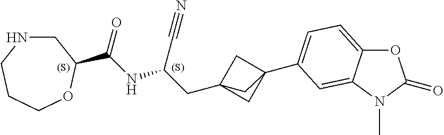 |
| 101e | 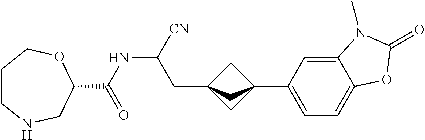 [SMILES: Cn1c(=O)oc2ccc([C@]34C[C@](CC(C#N)NC(=O)[C@@H]5CNCCCO5)(C3)C4)cc21] |
| 102 | 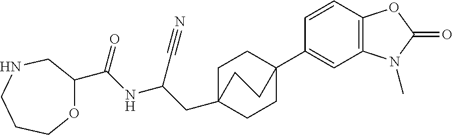 [SMILES: Cn1c(=O)oc2ccc(C34CCC(CC(C#N)NC(=O)C5CNCCCO5)(CC3)CC4)cc21] |
| 102a | 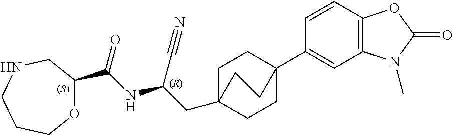 |
| 102b | 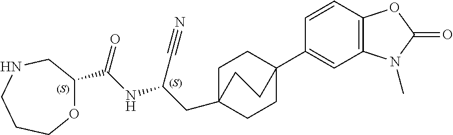 |
| 102c | 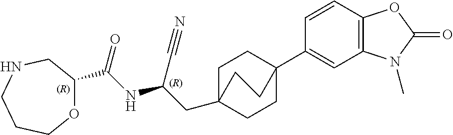 |
| 102d | 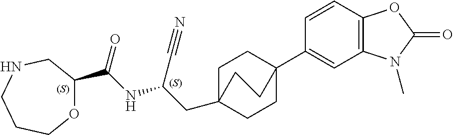 |
| 103 | 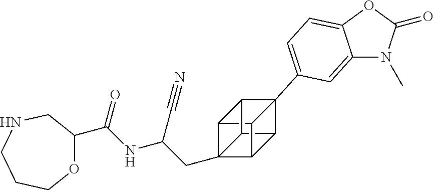 [SMILES: Cn1c(=O)oc2ccc(C34C5C6C3C3C4C5C63CC(C#N)NC(=O)C3CNCCCO3)cc21] |
| 103a | 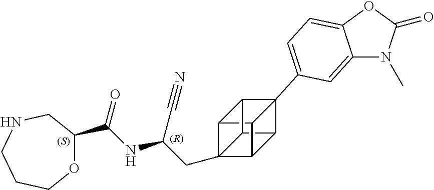 |
| 103b | 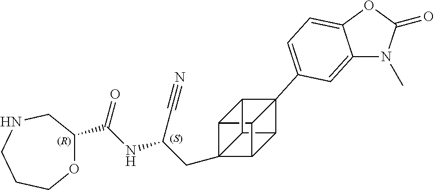 |
| 103c | 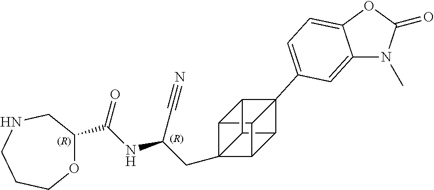 |
| 103d | 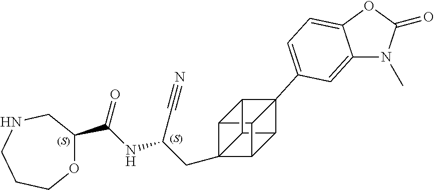 |
| 104 | 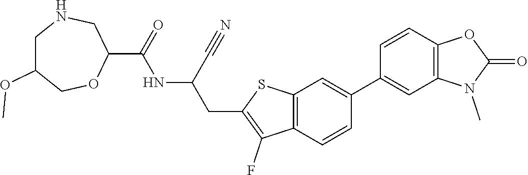 [SMILES: COC1CNCC(C(=O)NC(C#N)Cc2sc3cc(-c4ccc5oc(=O)n(C)c5c4)ccc3c2F)OC1] |
| 104a | 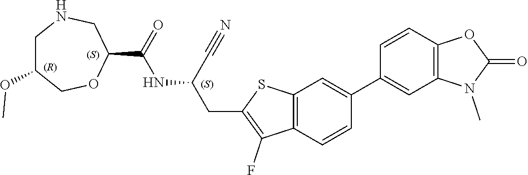 |
| 104b | 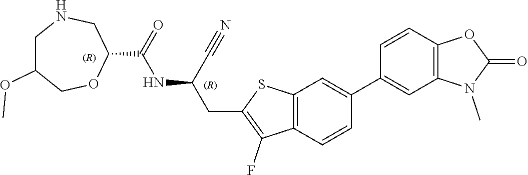 |
| 104c | 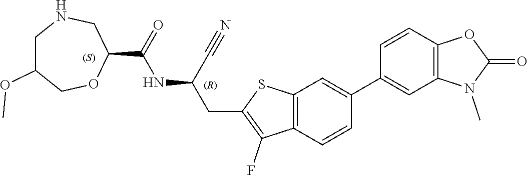 |
| 104d | 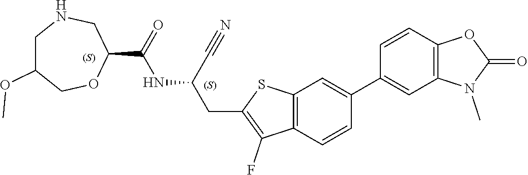 |
| 104e | 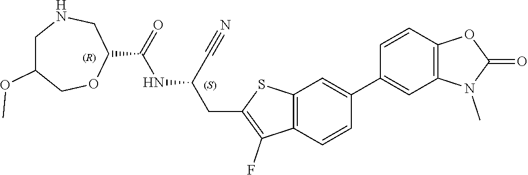 |
| 105 | 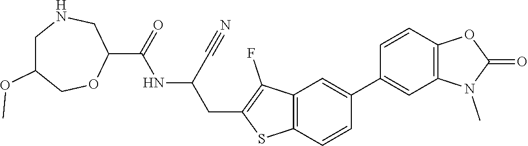 [SMILES: COC1CNCC(C(=O)NC(C#N)Cc2sc3ccc(-c4ccc5oc(=O)n(C)c5c4)cc3c2F)OC1] |
| 105a | 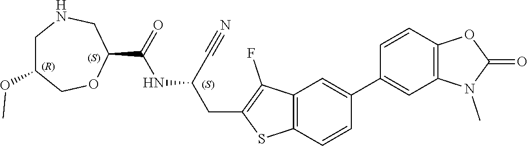 |
| 105b | 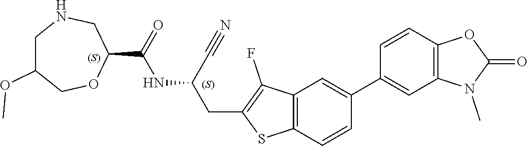 |
| 105c | 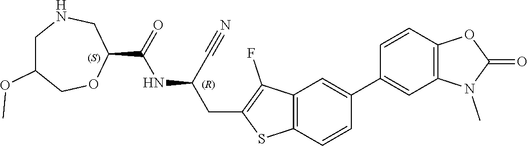 |
| 105d | 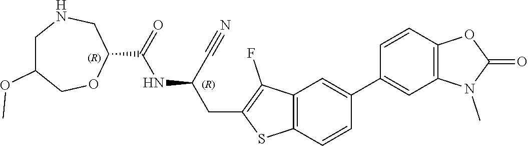 |
| 105e | 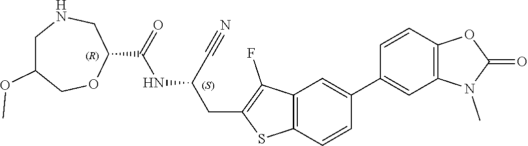 |
| 106 | 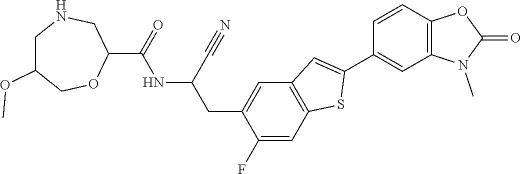 [SMILES: COC1CNCC(C(=O)NC(C#N)Cc2cc3cc(-c4ccc5oc(=O)n(C)c5c4)sc3cc2F)OC1] |
| 106a | 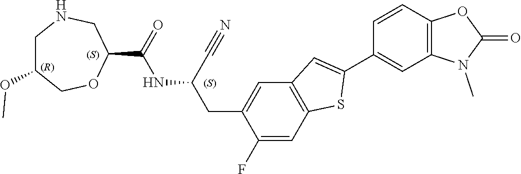 |
| 106b | 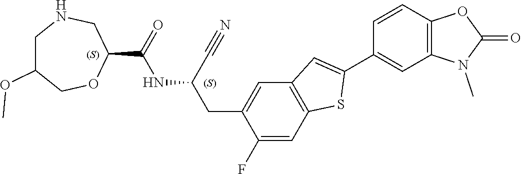 |
| 106c | 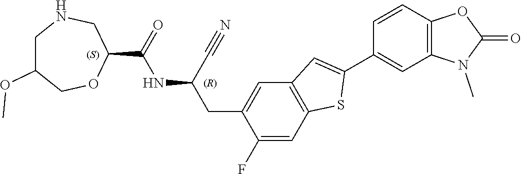 |
| 106d | 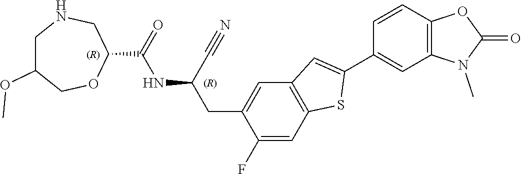 |
| 106e | 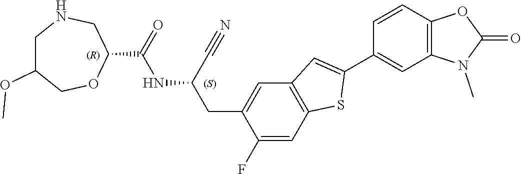 |
| 107 | 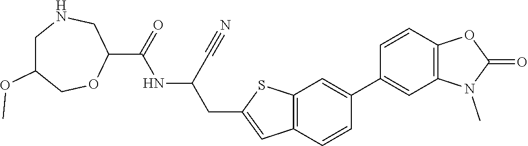 [SMILES: COC1CNCC(C(=O)NC(C#N)Cc2cc3ccc(-c4ccc5oc(=O)n(C)c5c4)cc3s2)OC1] |
| 107a | 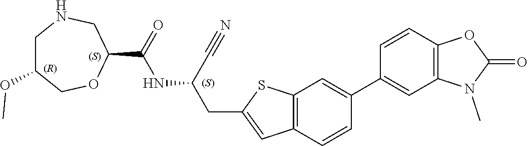 |
| 107b | 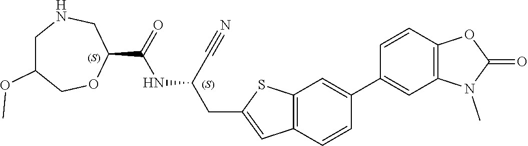 |
| 107c | 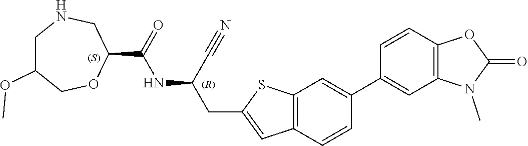 |
| 107d | 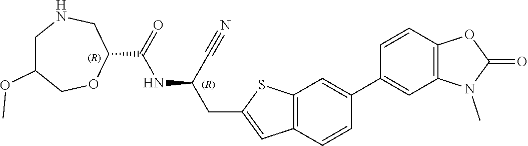 |
| 107e | 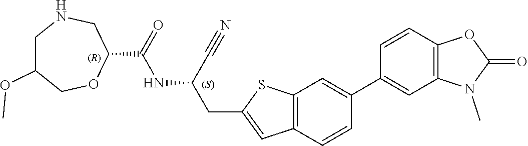 |
| 108 | 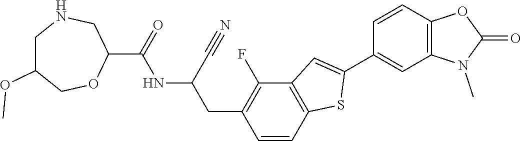 [SMILES: COC1CNCC(C(=O)NC(C#N)Cc2ccc3sc(-c4ccc5oc(=O)n(C)c5c4)cc3c2F)OC1] |
| 108a | 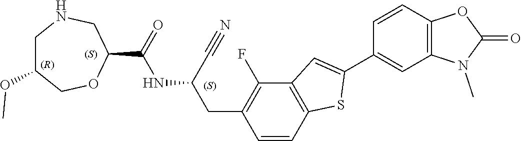 |
| 108b | 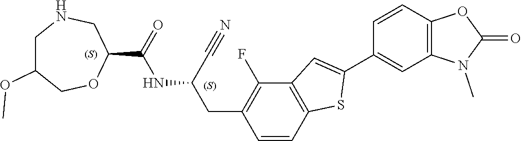 |
| 108c | 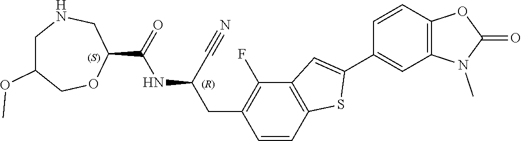 |
| 108d | 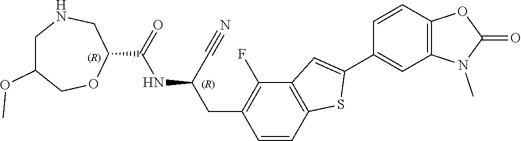 |
| 108e | 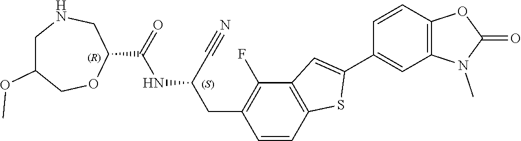 |
| 109 | 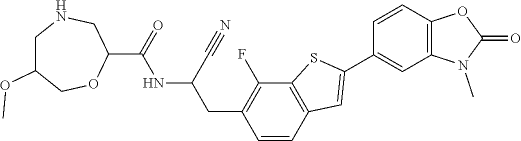 [SMILES: COC1CNCC(C(=O)NC(C#N)Cc2ccc3cc(-c4ccc5oc(=O)n(C)c5c4)sc3c2F)OC1] |
| 109a | 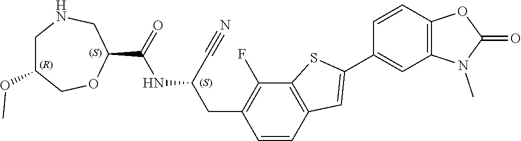 |
| 109b | 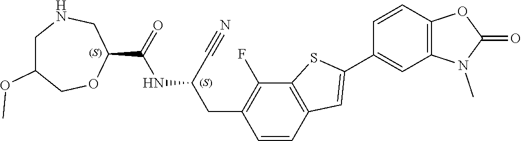 |
| 109c | 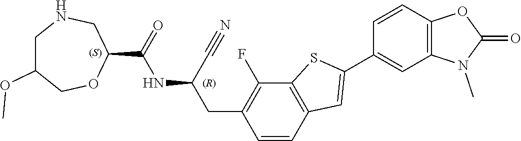 |
| 109d | 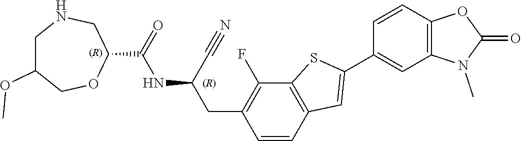 |
| 109e | 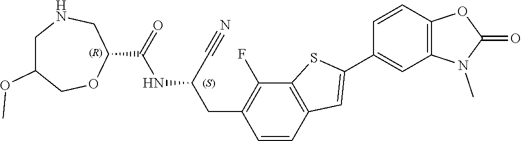 |
| 110 | 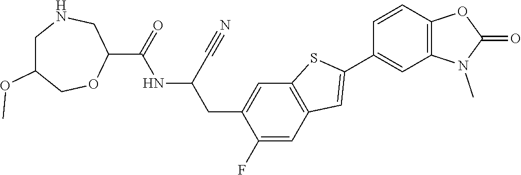 [SMILES: COC1CNCC(C(=O)NC(C#N)Cc2cc3sc(-c4ccc5oc(=O)n(C)c5c4)cc3cc2F)OC1] |
| 110a | 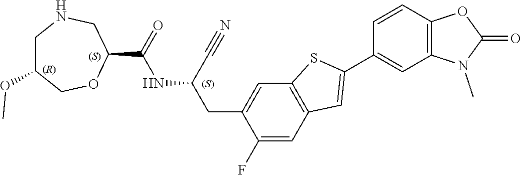 |
| 110b | 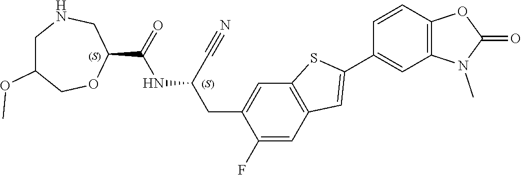 |
| 110c | 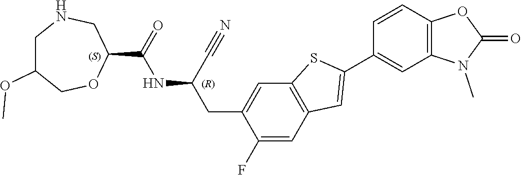 |
| 110d | 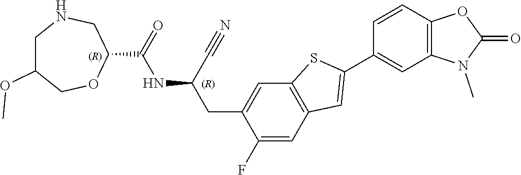 |
| 110e | 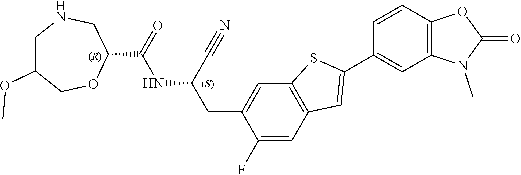 |
| 111 | 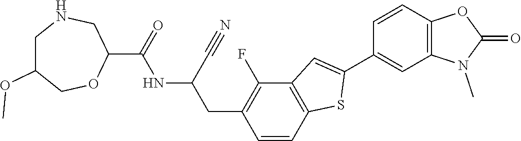 [SMILES: COC1CNCC(C(=O)NC(C#N)Cc2ccc3sc(-c4ccc5oc(=O)n(C)c5c4)cc3c2F)OC1] |
| 111a | 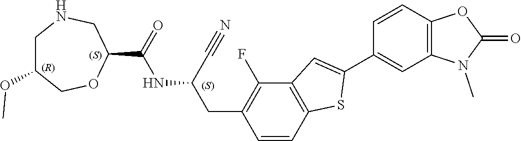 |
| 111b | 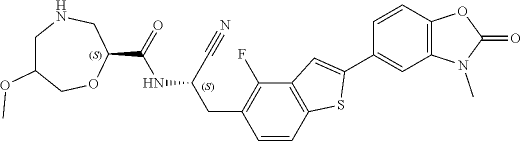 |
| 111c | 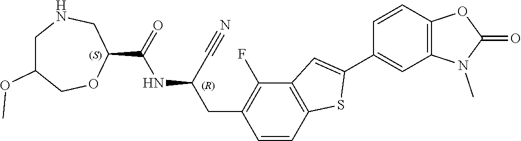 |
| 111d | 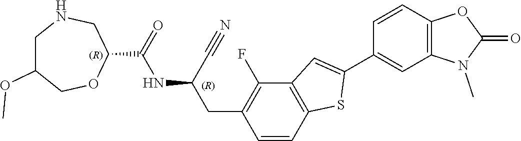 |
| 111e | 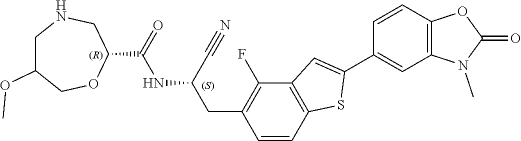 |
| 112 | 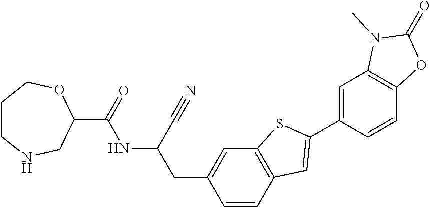 [SMILES: Cn1c(=O)oc2ccc(-c3cc4ccc(CC(C#N)NC(=O)C5CNCCCO5)cc4s3)cc21] |
| 112a | 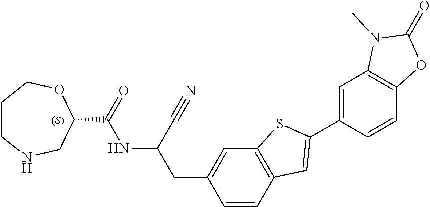 |
| 112b | 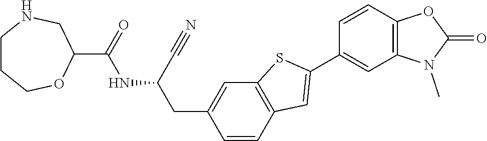 [SMILES: Cn1c(=O)oc2ccc(-c3cc4ccc(C[C@@H](C#N)NC(=O)C5CNCCCO5)cc4s3)cc21] |
| 113 | 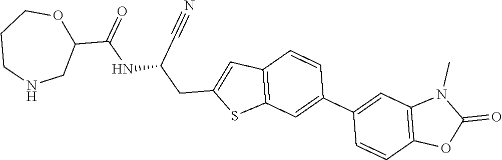 [SMILES: Cn1c(=O)oc2ccc(-c3ccc4cc(C[C@@H](C#N)NC(=O)C5CNCCCO5)sc4c3)cc21] |
| 113a | 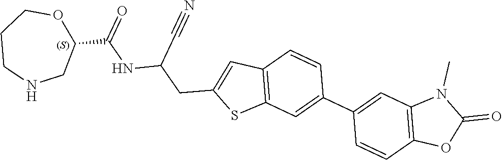 |
| 113b | 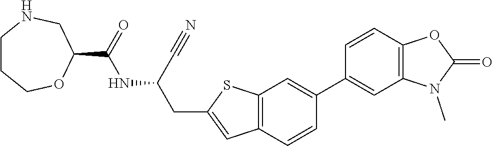 [SMILES: Cn1c(=O)oc2ccc(-c3ccc4cc(C[C@@H](C#N)NC(=O)[C@@H]5CNCCCO5)sc4c3)cc21] |
| 114 | 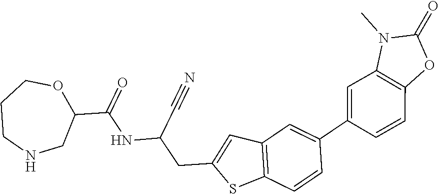 [SMILES: Cn1c(=O)oc2ccc(-c3ccc4sc(CC(C#N)NC(=O)C5CNCCCO5)cc4c3)cc21] |
| 114a | 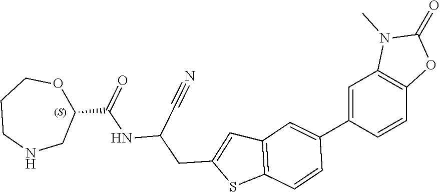 |
| 114b | 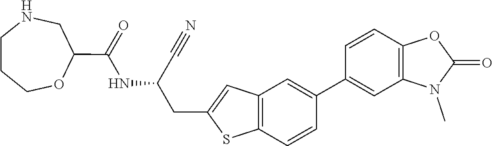 [SMILES: Cn1c(=O)oc2ccc(-c3ccc4sc(C[C@@H](C#N)NC(=O)C5CNCCCO5)cc4c3)cc21] |
| 115 | 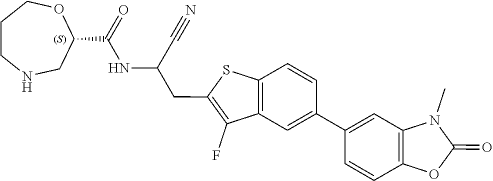 |
| 115a | 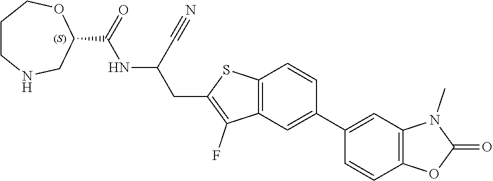 |
| 115b | 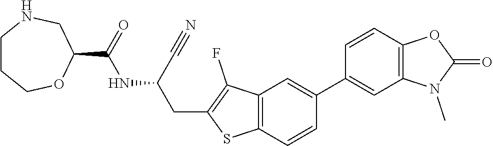 [SMILES: Cn1c(=O)oc2ccc(-c3ccc4sc(C[C@@H](C#N)NC(=O)[C@@H]5CNCCCO5)c(F)c4c3)cc21] |
| 116 | 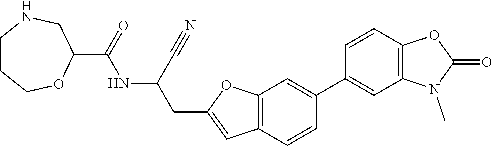 [SMILES: Cn1c(=O)oc2ccc(-c3ccc4cc(CC(C#N)NC(=O)C5CNCCCO5)oc4c3)cc21] |
| 116a | 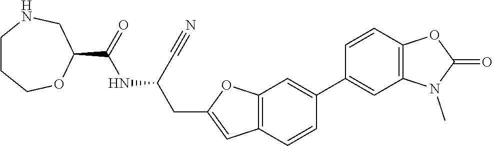 [SMILES: Cn1c(=O)oc2ccc(-c3ccc4cc(C[C@@H](C#N)NC(=O)[C@@H]5CNCCCO5)oc4c3)cc21] |
| 116b | 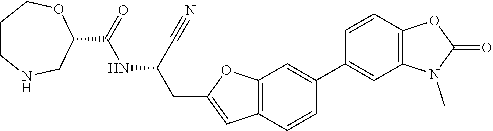 [SMILES: Cn1c(=O)oc2ccc(-c3ccc4cc(C[C@@H](C#N)NC(=O)[C@@H]5CNCCCO5)oc4c3)cc21] |
| 117 | 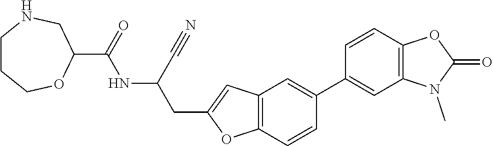 [SMILES: Cn1c(=O)oc2ccc(-c3ccc4oc(CC(C#N)NC(=O)C5CNCCCO5)cc4c3)cc21] |
| 117a | 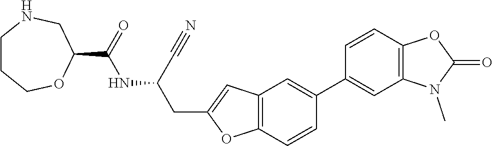 [SMILES: Cn1c(=O)oc2ccc(-c3ccc4oc(C[C@@H](C#N)NC(=O)[C@@H]5CNCCCO5)cc4c3)cc21] |
| 117b | 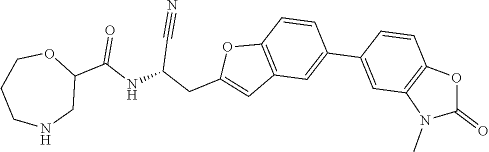 [SMILES: Cn1c(=O)oc2ccc(-c3ccc4oc(C[C@@H](C#N)NC(=O)C5CNCCCO5)cc4c3)cc21] |
| 118 | 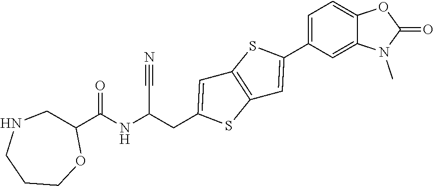 [SMILES: Cn1c(=O)oc2ccc(-c3cc4sc(CC(C#N)NC(=O)C5CNCCCO5)cc4s3)cc21] |
| 118a | 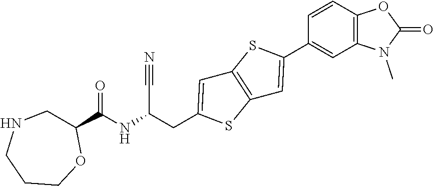 [SMILES: Cn1c(=O)oc2ccc(-c3cc4sc(C[C@@H](C#N)NC(=O)[C@@H]5CNCCCO5)cc4s3)cc21] |
| 118b | 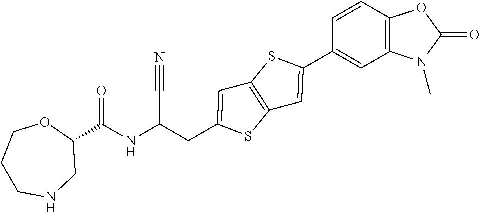 [SMILES: Cn1c(=O)oc2ccc(-c3cc4sc(CC(C#N)NC(=O)[C@@H]5CNCCCO5)cc4s3)cc21] |
| 119 | 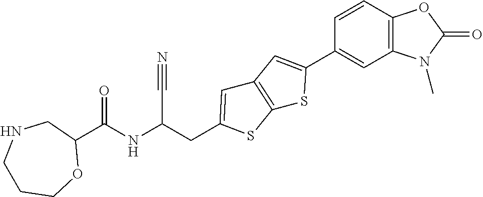 [SMILES: Cn1c(=O)oc2ccc(-c3cc4cc(CC(C#N)NC(=O)C5CNCCCO5)sc4s3)cc21] |
| 119a | 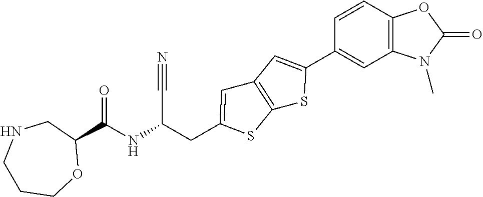 [SMILES: Cn1c(=O)oc2ccc(-c3cc4cc(C[C@@H](C#N)NC(=O)[C@@H]5CNCCCO5)sc4s3)cc21] |
| 120 | 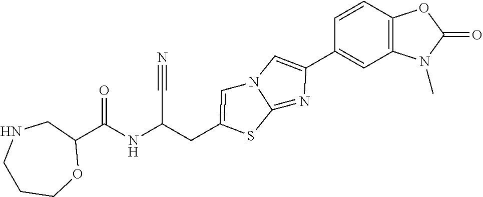 [SMILES: Cn1c(=O)oc2ccc(-c3cn4cc(CC(C#N)NC(=O)C5CNCCCO5)sc4n3)cc21] |
| 120a | 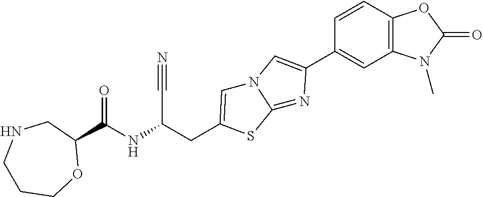 [SMILES: Cn1c(=O)oc2ccc(-c3cn4cc(C[C@@H](C#N)NC(=O)[C@@H]5CNCCCO5)sc4n3)cc21] |
| 121 | 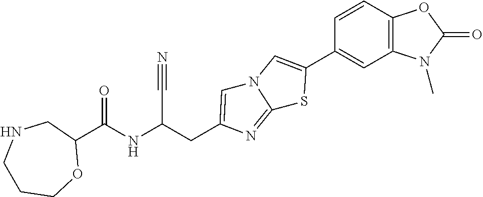 [SMILES: Cn1c(=O)oc2ccc(-c3cn4cc(CC(C#N)NC(=O)C5CNCCCO5)nc4s3)cc21] |
| 121a | 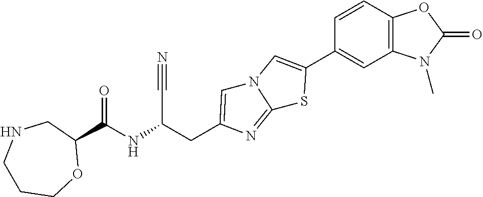 [SMILES: Cn1c(=O)oc2ccc(-c3cn4cc(C[C@@H](C#N)NC(=O)[C@@H]5CNCCCO5)nc4s3)cc21] |
| 122 | 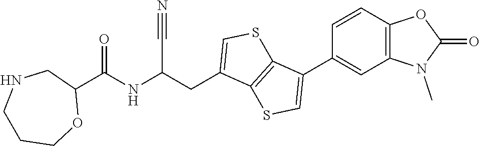 [SMILES: Cn1c(=O)oc2ccc(-c3csc4c(CC(C#N)NC(=O)C5CNCCCO5)csc34)cc21] |
| 122a | 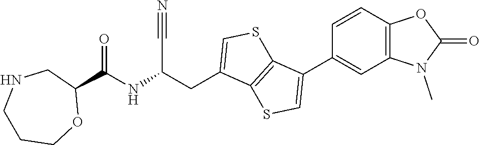 [SMILES: Cn1c(=O)oc2ccc(-c3csc4c(C[C@@H](C#N)NC(=O)[C@@H]5CNCCCO5)csc34)cc21] |
| 123 | 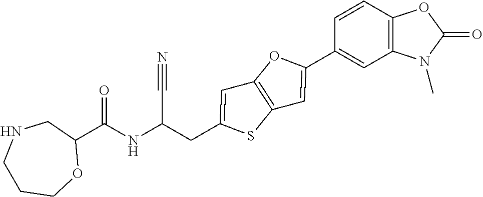 [SMILES: Cn1c(=O)oc2ccc(-c3cc4sc(CC(C#N)NC(=O)C5CNCCCO5)cc4o3)cc21] |
| 123a | 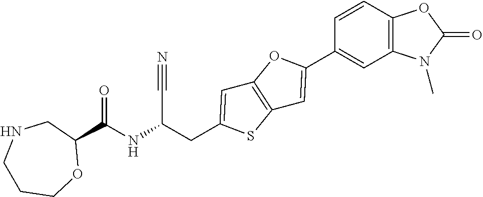 [SMILES: Cn1c(=O)oc2ccc(-c3cc4sc(C[C@@H](C#N)NC(=O)[C@@H]5CNCCCO5)cc4o3)cc21] |
| 124 | 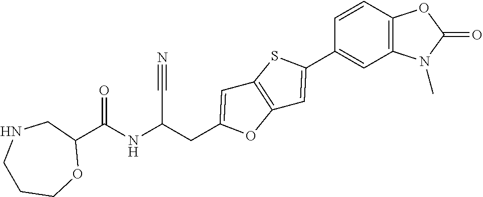 [SMILES: Cn1c(=O)oc2ccc(-c3cc4oc(CC(C#N)NC(=O)C5CNCCCO5)cc4s3)cc21] |
| 124a | 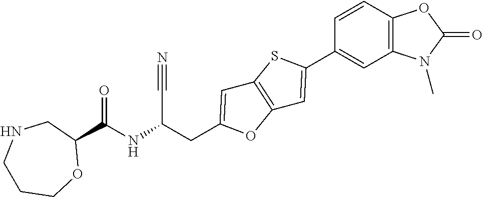 [SMILES: Cn1c(=O)oc2ccc(-c3cc4oc(C[C@@H](C#N)NC(=O)[C@@H]5CNCCCO5)cc4s3)cc21] |
| 125 | 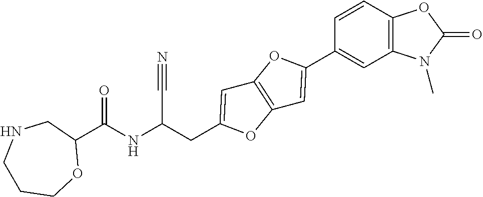 [SMILES: Cn1c(=O)oc2ccc(-c3cc4oc(CC(C#N)NC(=O)C5CNCCCO5)cc4o3)cc21] |
| 125a | 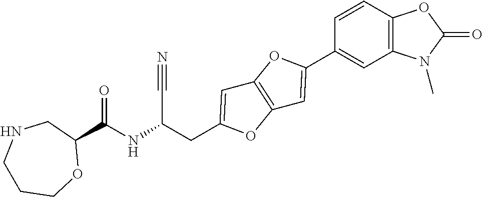 [SMILES: Cn1c(=O)oc2ccc(-c3cc4oc(C[C@@H](C#N)NC(=O)[C@@H]5CNCCCO5)cc4o3)cc21] |
| 126 | 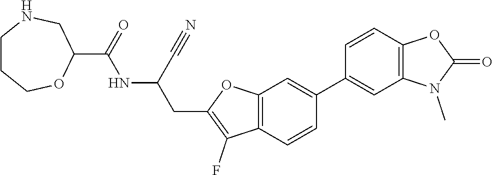 [SMILES: Cn1c(=O)oc2ccc(-c3ccc4c(F)c(CC(C#N)NC(=O)C5CNCCCO5)oc4c3)cc21] |
| 126a | 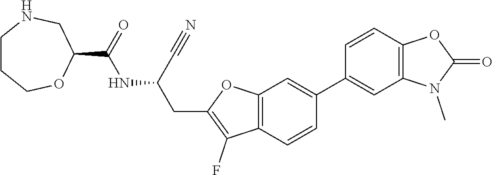 [SMILES: Cn1c(=O)oc2ccc(-c3ccc4c(F)c(C[C@@H](C#N)NC(=O)[C@@H]5CNCCCO5)oc4c3)cc21] |
| 127 | 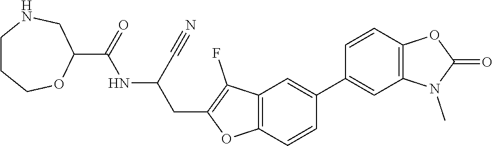 [SMILES: Cn1c(=O)oc2ccc(-c3ccc4oc(CC(C#N)NC(=O)C5CNCCCO5)c(F)c4c3)cc21] |
| 127a | 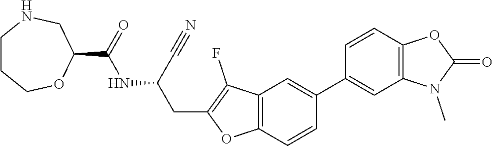 [SMILES: Cn1c(=O)oc2ccc(-c3ccc4oc(C[C@@H](C#N)NC(=O)[C@@H]5CNCCCO5)c(F)c4c3)cc21] |
| 128 | 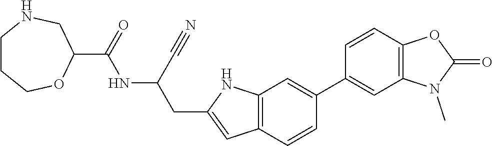 [SMILES: Cn1c(=O)oc2ccc(-c3ccc4cc(CC(C#N)NC(=O)C5CNCCCO5)[nH]c4c3)cc21] |
| 128a | 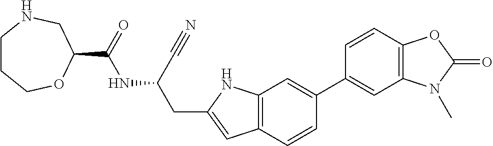 [SMILES: Cn1c(=O)oc2ccc(-c3ccc4cc(C[C@@H](C#N)NC(=O)[C@@H]5CNCCCO5)[nH]c4c3)cc21] |
| 128b | 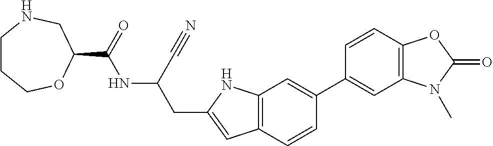 [SMILES: Cn1c(=O)oc2ccc(-c3ccc4cc(CC(C#N)NC(=O)[C@@H]5CNCCCO5)[nH]c4c3)cc21] |
| 129 | 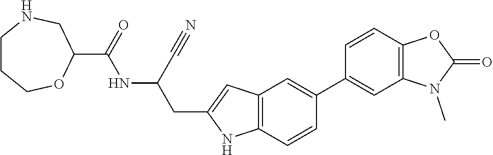 [SMILES: Cn1c(=O)oc2ccc(-c3ccc4[nH]c(CC(C#N)NC(=O)C5CNCCCO5)cc4c3)cc21] |
| 129a | 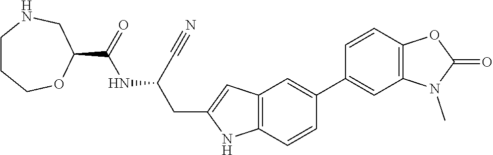 [SMILES: Cn1c(=O)oc2ccc(-c3ccc4[nH]c(C[C@@H](C#N)NC(=O)[C@@H]5CNCCCO5)cc4c3)cc21] |
| 129b | 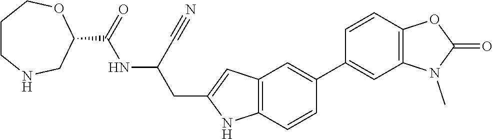 [SMILES: Cn1c(=O)oc2ccc(-c3ccc4[nH]c(CC(C#N)NC(=O)[C@@H]5CNCCCO5)cc4c3)cc21] |
| 130 | 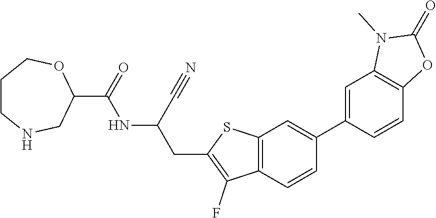 [SMILES: Cn1c(=O)oc2ccc(-c3ccc4c(F)c(CC(C#N)NC(=O)C5CNCCCO5)sc4c3)cc21] |
| 130a | 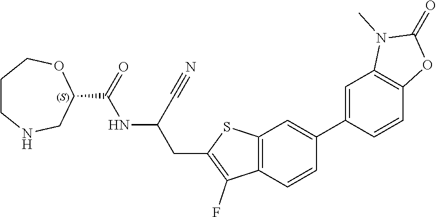 |
| 130b | 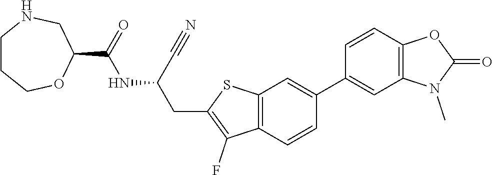 [SMILES: Cn1c(=O)oc2ccc(-c3ccc4c(F)c(C[C@@H](C#N)NC(=O)[C@@H]5CNCCCO5)sc4c3)cc21] |
| 131 | 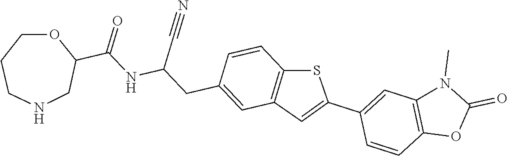 [SMILES: Cn1c(=O)oc2ccc(-c3cc4cc(CC(C#N)NC(=O)C5CNCCCO5)ccc4s3)cc21] |
| 131a | 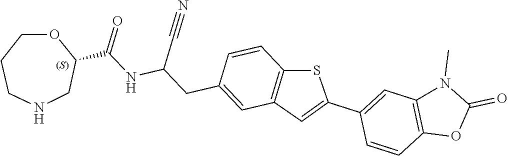 |
| 131b | 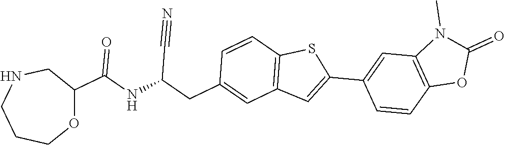 [SMILES: Cn1c(=O)oc2ccc(-c3cc4cc(C[C@@H](C#N)NC(=O)C5CNCCCO5)ccc4s3)cc21] |
| 132 | 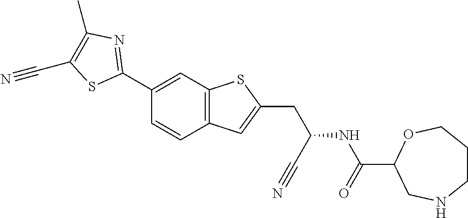 [SMILES: Cc1nc(-c2ccc3cc(C[C@@H](C#N)NC(=O)C4CNCCCO4)sc3c2)sc1C#N] |
| 132a | 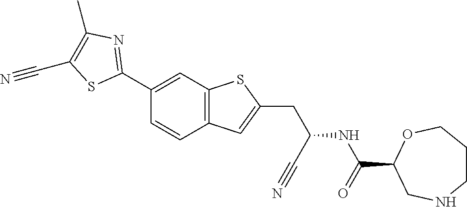 [SMILES: Cc1nc(-c2ccc3cc(C[C@@H](C#N)NC(=O)[C@@H]4CNCCCO4)sc3c2)sc1C#N] |
| 133 | 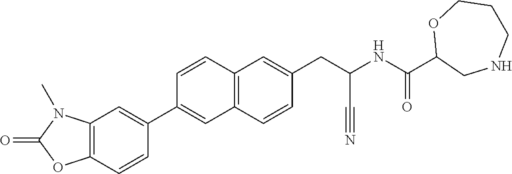 [SMILES: Cn1c(=O)oc2ccc(-c3ccc4cc(CC(C#N)NC(=O)C5CNCCCO5)ccc4c3)cc21] |
| 133a | 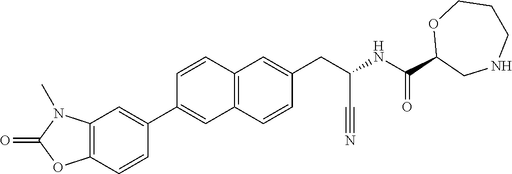 [SMILES: Cn1c(=O)oc2ccc(-c3ccc4cc(C[C@@H](C#N)NC(=O)[C@@H]5CNCCCO5)ccc4c3)cc21] |
| 133b | 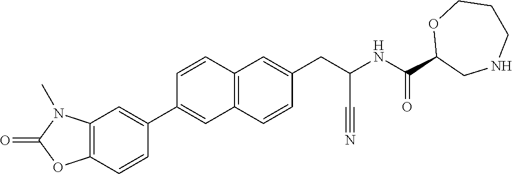 [SMILES: Cn1c(=O)oc2ccc(-c3ccc4cc(CC(C#N)NC(=O)[C@@H]5CNCCCO5)ccc4c3)cc21] |
| 134 | 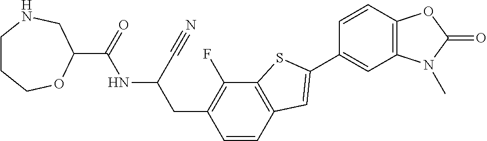 [SMILES: Cn1c(=O)oc2ccc(-c3cc4ccc(CC(C#N)NC(=O)C5CNCCCO5)c(F)c4s3)cc21] |
| 134a | 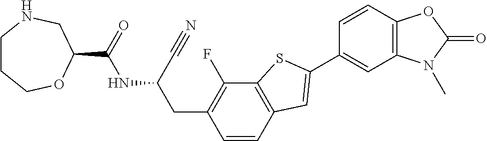 [SMILES: Cn1c(=O)oc2ccc(-c3cc4ccc(C[C@@H](C#N)NC(=O)[C@@H]5CNCCCO5)c(F)c4s3)cc21] |
| 134b | 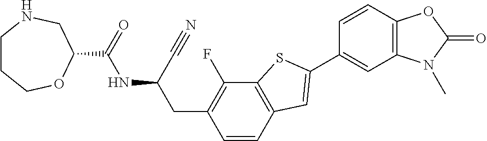 [SMILES: Cn1c(=O)oc2ccc(-c3cc4ccc(C[C@H](C#N)NC(=O)[C@H]5CNCCCO5)c(F)c4s3)cc21] |
| 134c | 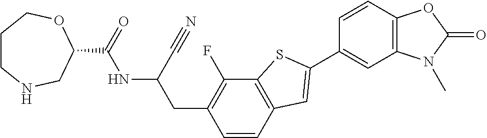 [SMILES: Cn1c(=O)oc2ccc(-c3cc4ccc(CC(C#N)NC(=O)[C@@H]5CNCCCO5)c(F)c4s3)cc21] |
| 134c | 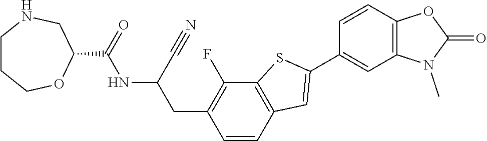 [SMILES: Cn1c(=O)oc2ccc(-c3cc4ccc(CC(C#N)NC(=O)[C@H]5CNCCCO5)c(F)c4s3)cc21] |
| 135 | 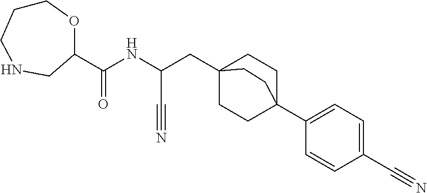 [SMILES: N#Cc1ccc(C23CCC(CC(C#N)NC(=O)C4CNCCCO4)(CC2)CC3)cc1] |
| 135a | 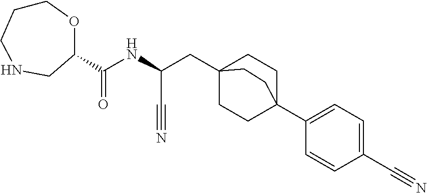 [SMILES: N#Cc1ccc(C23CCC(C[C@@H](C#N)NC(=O)[C@@H]4CNCCCO4)(CC2)CC3)cc1] |
| 135b | 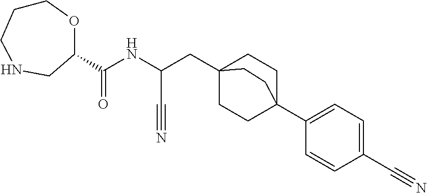 [SMILES: N#Cc1ccc(C23CCC(CC(C#N)NC(=O)[C@@H]4CNCCCO4)(CC2)CC3)cc1] |
| 136 | 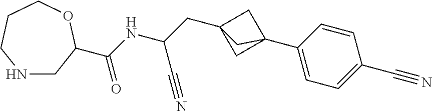 [SMILES: N#Cc1ccc(C23CC(CC(C#N)NC(=O)C4CNCCCO4)(C2)C3)cc1] |
| 136a | 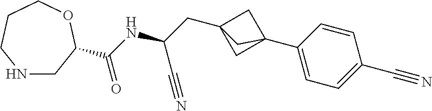 [SMILES: N#Cc1ccc(C23CC(C[C@@H](C#N)NC(=O)[C@@H]4CNCCCO4)(C2)C3)cc1] |
| 136b | 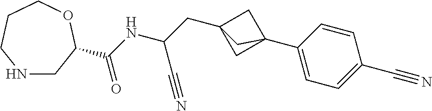 [SMILES: N#Cc1ccc(C23CC(CC(C#N)NC(=O)[C@@H]4CNCCCO4)(C2)C3)cc1] |
| 137 | 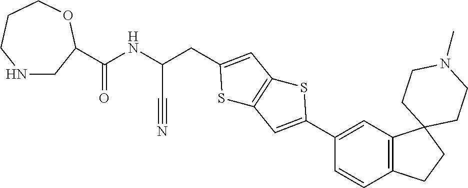 [SMILES: CN1CCC2(CCc3ccc(-c4cc5sc(CC(C#N)NC(=O)C6CNCCCO6)cc5s4)cc32)CC1] |
| 137a | 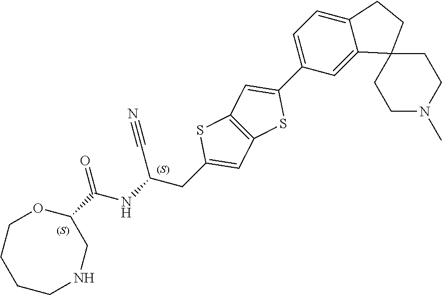 |
| 137b | 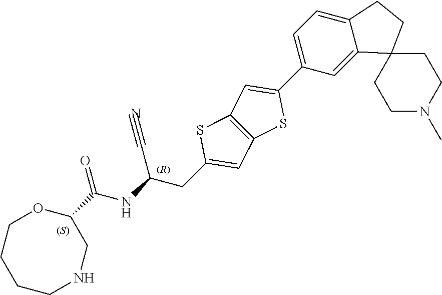 |
| 138 | 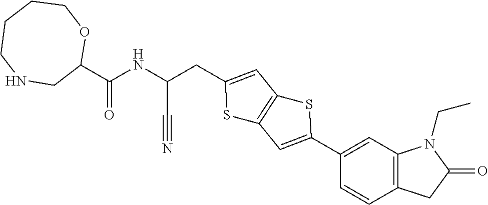 [SMILES: CCN1C(=O)Cc2ccc(-c3cc4sc(CC(C#N)NC(=O)C5CNCCCCO5)cc4s3)cc21] |
| 138a | 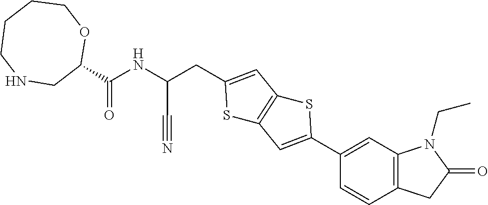 [SMILES: CCN1C(=O)Cc2ccc(-c3cc4sc(CC(C#N)NC(=O)[C@@H]5CNCCCCO5)cc4s3)cc21] |
| 138b | 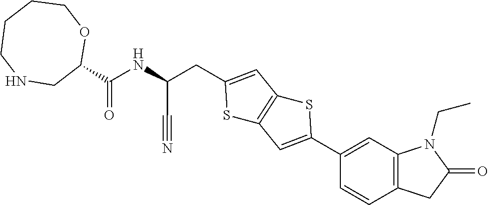 [SMILES: CCN1C(=O)Cc2ccc(-c3cc4sc(C[C@@H](C#N)NC(=O)[C@@H]5CNCCCCO5)cc4s3)cc21] |
| 138c | 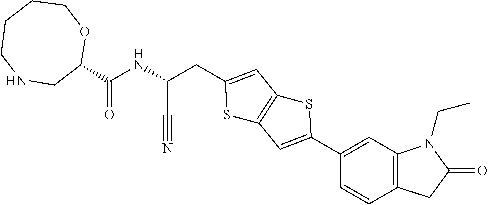 [SMILES: CCN1C(=O)Cc2ccc(-c3cc4sc(C[C@H](C#N)NC(=O)[C@@H]5CNCCCCO5)cc4s3)cc21] |
| 139 | 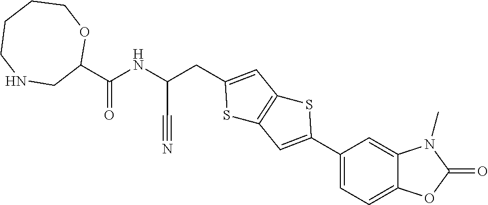 [SMILES: Cn1c(=O)oc2ccc(-c3cc4sc(CC(C#N)NC(=O)C5CNCCCCO5)cc4s3)cc21] |
| 139a | 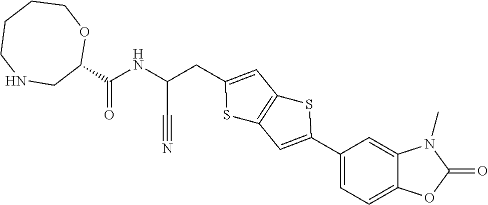 [SMILES: Cn1c(=O)oc2ccc(-c3cc4sc(CC(C#N)NC(=O)[C@@H]5CNCCCCO5)cc4s3)cc21] |
| 139b | 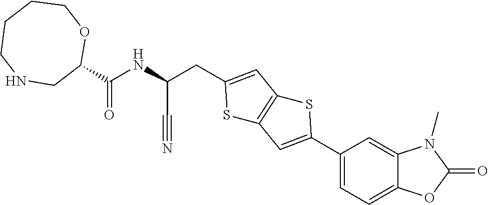 [SMILES: Cn1c(=O)oc2ccc(-c3cc4sc(C[C@@H](C#N)NC(=O)[C@@H]5CNCCCCO5)cc4s3)cc21] |
| 139c | 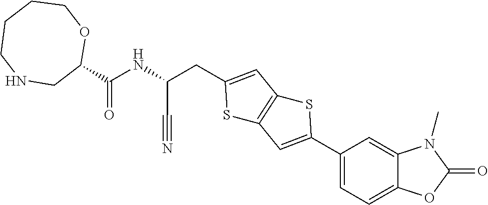 [SMILES: Cn1c(=O)oc2ccc(-c3cc4sc(C[C@H](C#N)NC(=O)[C@@H]5CNCCCCO5)cc4s3)cc21] |
| 141 | 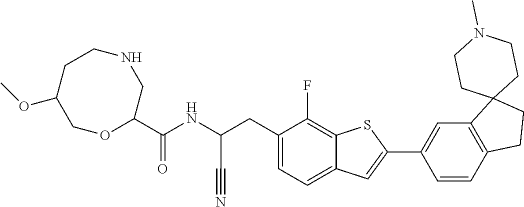 [SMILES: COC1CCNCC(C(=O)NC(C#N)Cc2ccc3cc(-c4ccc5c(c4)C4(CC5)CCN(C)CC4)sc3c2F)OC1] |
| 141a | 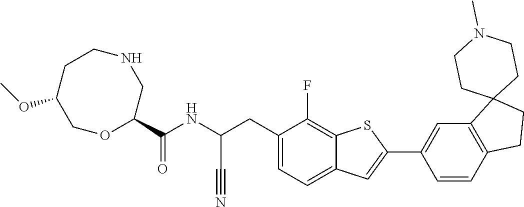 [SMILES: CO[C@@H]1CCNC[C@@H](C(=O)NC(C#N)Cc2ccc3cc(-c4ccc5c(c4)C4(CC5)CCN(C)CC4)sc3c2F)OC1] |
| 141b | 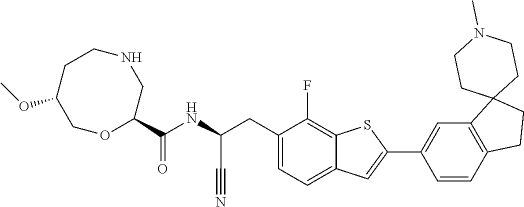 [SMILES: CO[C@@H]1CCNC[C@@H](C(=O)N[C@H](C#N)Cc2ccc3cc(-c4ccc5c(c4)C4(CC5)CCN(C)CC4)sc3c2F)OC1] |
| 141c | 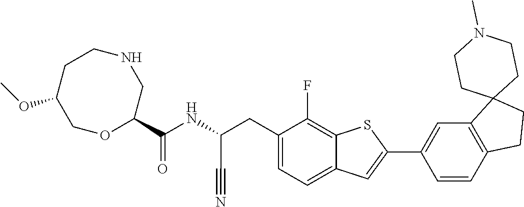 [SMILES: CO[C@@H]1CCNC[C@@H](C(=O)N[C@@H](C#N)Cc2ccc3cc(-c4ccc5c(c4)C4(CC5)CCN(C)CC4)sc3c2F)OC1] |
| 142 | 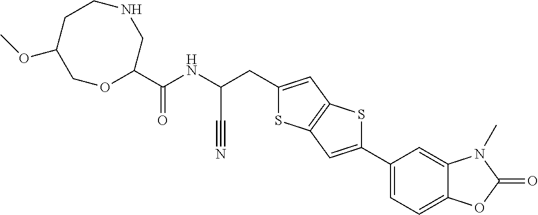 [SMILES: COC1CCNCC(C(=O)NC(C#N)Cc2cc3sc(-c4ccc5oc(=O)n(C)c5c4)cc3s2)OC1] |
| 142a | 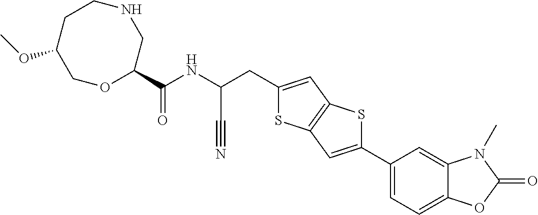 [SMILES: CO[C@@H]1CCNC[C@@H](C(=O)NC(C#N)Cc2cc3sc(-c4ccc5oc(=O)n(C)c5c4)cc3s2)OC1] |
| 142b | 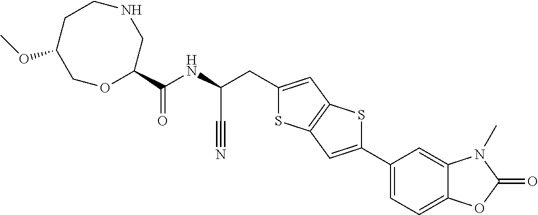 [SMILES: CO[C@@H]1CCNC[C@@H](C(=O)N[C@H](C#N)Cc2cc3sc(-c4ccc5oc(=O)n(C)c5c4)cc3s2)OC1] |
| 142c | 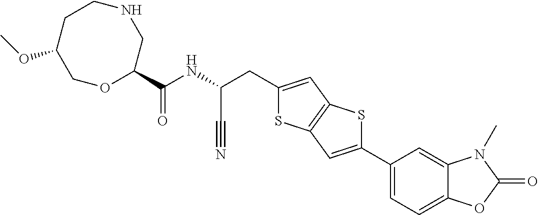 [SMILES: CO[C@@H]1CCNC[C@@H](C(=O)N[C@@H](C#N)Cc2cc3sc(-c4ccc5oc(=O)n(C)c5c4)cc3s2)OC1] |
| 143 | 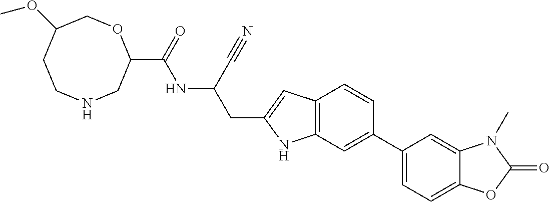 [SMILES: COC1CCNCC(C(=O)NC(C#N)Cc2cc3ccc(-c4ccc5oc(=O)n(C)c5c4)cc3[nH]2)OC1] |
| 143a | 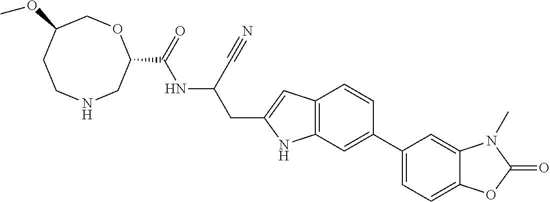 [SMILES: CO[C@@H]1CCNC[C@@H](C(=O)NC(C#N)Cc2cc3ccc(-c4ccc5oc(=O)n(C)c5c4)cc3[nH]2)OC1] |
| 143b | 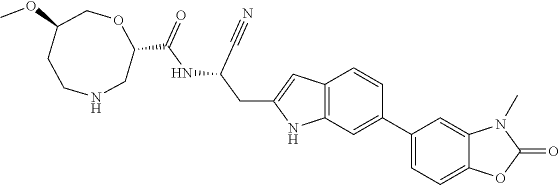 [SMILES: CO[C@@H]1CCNC[C@@H](C(=O)N[C@H](C#N)Cc2cc3ccc(-c4ccc5oc(=O)n(C)c5c4)cc3[nH]2)OC1] |
| 143c | 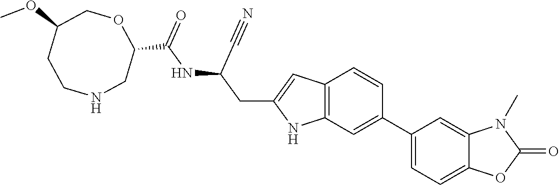 [SMILES: CO[C@@H]1CCNC[C@@H](C(=O)N[C@@H](C#N)Cc2cc3ccc(-c4ccc5oc(=O)n(C)c5c4)cc3[nH]2)OC1] |
| 144 | 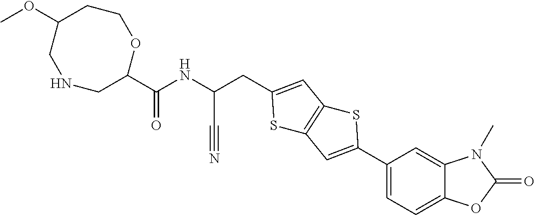 [SMILES: COC1CCOC(C(=O)NC(C#N)Cc2cc3sc(-c4ccc5oc(=O)n(C)c5c4)cc3s2)CNC1] |
| 144a | 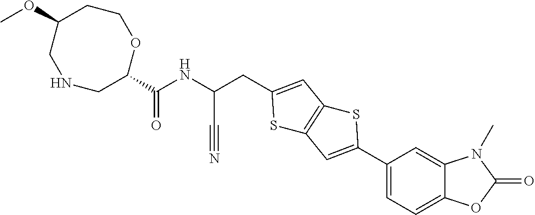 [SMILES: CO[C@H]1CCO[C@H](C(=O)NC(C#N)Cc2cc3sc(-c4ccc5oc(=O)n(C)c5c4)cc3s2)CNC1] |
| 144b | 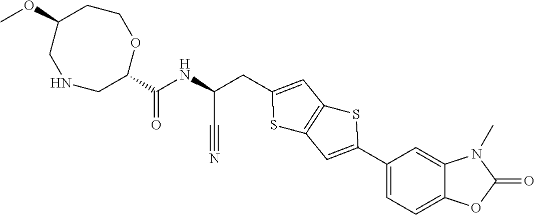 [SMILES: CO[C@H]1CCO[C@H](C(=O)N[C@H](C#N)Cc2cc3sc(-c4ccc5oc(=O)n(C)c5c4)cc3s2)CNC1] |
| 144c | 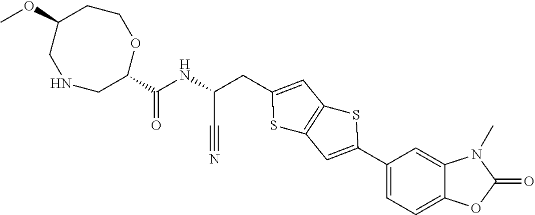 [SMILES: CO[C@H]1CCO[C@H](C(=O)N[C@@H](C#N)Cc2cc3sc(-c4ccc5oc(=O)n(C)c5c4)cc3s2)CNC1] |
| 145 | 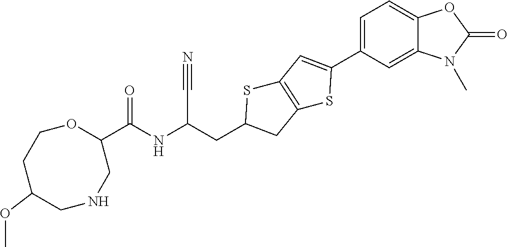 [SMILES: COC1CCOC(C(=O)NC(C#N)CC2Cc3sc(-c4ccc5oc(=O)n(C)c5c4)cc3S2)CNC1] |
| 145a | 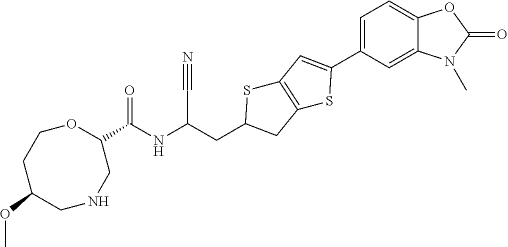 |
| 145b | 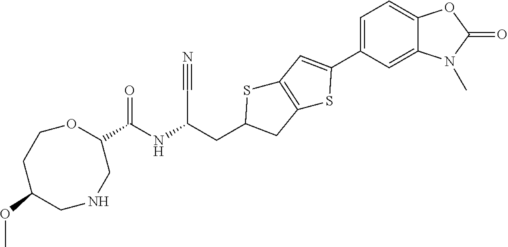 |
| 145c | 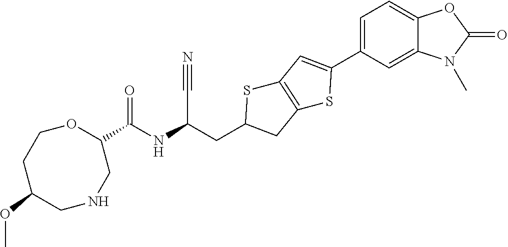 |
| 146 | 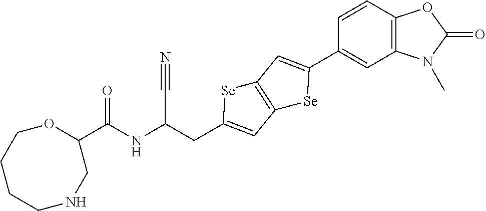 [SMILES: Cn1c(=O)oc2ccc(-c3cc4[se]c(CC(C#N)NC(=O)C5CNCCCCO5)cc4[se]3)cc21] |
| 146a | 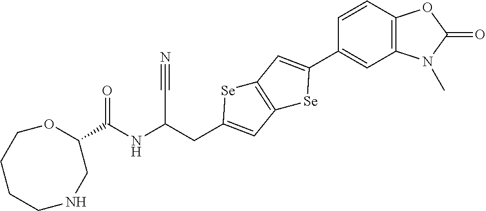 [SMILES: Cn1c(=O)oc2ccc(-c3cc4[se]c(CC(C#N)NC(=O)[C@@H]5CNCCCCO5)cc4[se]3)cc21] |
| 146b | 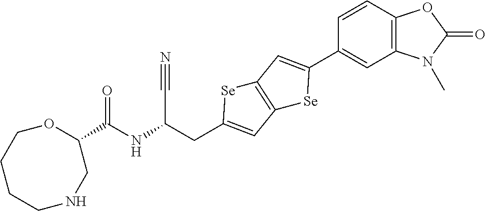 [SMILES: Cn1c(=O)oc2ccc(-c3cc4[se]c(C[C@@H](C#N)NC(=O)[C@@H]5CNCCCCO5)cc4[se]3)cc21] |
| 146c | 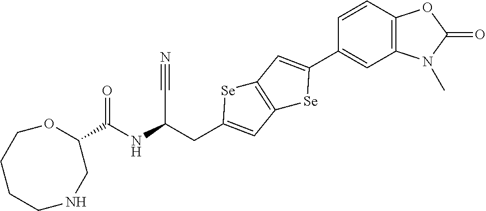 [SMILES: Cn1c(=O)oc2ccc(-c3cc4[se]c(C[C@H](C#N)NC(=O)[C@@H]5CNCCCCO5)cc4[se]3)cc21] |

### Compositions

The compounds of Formula (I), (II), (III), (IV), (V), (VI), (VII), (VIII), (IX), (X), (XI), (XII), (XIII), (XIV), (XV) or (XVI), or Table 1 or pharmaceutically acceptable salts thereof, or deuterated versions of the foregoing, may be used on their own but will generally be administered in the form of a pharmaceutical composition in which the Formula (I), (II) or (III), (III-A), (III-B) or Table 1 compound/salt (active ingredient) is in association with pharmaceutically acceptable adjuvant(s), diluents(s) or carrier(s). Conventional procedures for the selection and preparation of suitable pharmaceutical formulations are described in, for example, “Pharmaceuticals—The Science of Dosage Form Designs”, M. E. Aulton, Churchill Livingstone, 2nd Ed. 2002.

In embodiments, the present disclosure provides pharmaceutical composition(s) comprising a compound of Formula (I), (II), (III), (IV), (V), (VI), (VII), (VIII), (IX), (X), (XI), (XII), (XIII), (XIV), (XV) or (XVI), or Table 1 or a pharmaceutically acceptable salt thereof, or a deuterated form thereof, as hereinbefore defined in association with pharmaceutically acceptable adjuvant(s), diluent(s) or carrier(s).

The disclosure further provides a process for the preparation of a pharmaceutical composition of the disclosure which comprises mixing a compound of Formula (I), (II), (III), (IV), (V), (VI), (VII), (VIII), (IX), (X), (XI), (XII), (XIII), (XIV), (XV) or (XVI), or Table 1 or a pharmaceutically acceptable salt thereof, or a deuterated form thereof, as hereinbefore defined with a pharmaceutically acceptable adjuvant(s), diluents(s) or carrier(s).

The pharmaceutical compositions may be administered topically (e.g., to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g., by oral administration in the form of tablets, capsules, syrups, powders or granules; or by parenteral administration in the form of a sterile solution, suspension or emulsion for injection (including intravenous, subcutaneous, intramuscular, intravascular or infusion); or by rectal administration in the form of suppositories.

For oral administration the compound of the disclosure may be admixed with adjuvant(s), diluent(s) or carrier(s), for example, lactose, saccharose, sorbitol, mannitol; starch, for example, potato starch, corn starch or amylopectin; cellulose derivative; binder, for example, gelatine or polyvinylpyrrolidone; disintegrant, for example cellulose derivative, and/or lubricant, for example, magnesium stearate, calcium stearate, polyethylene glycol, wax, paraffin, and the like, and then compressed into tablets. If coated tablets are required, the cores, prepared as described above, may be coated with a suitable polymer dissolved or dispersed in water or readily volatile organic solvent(s). Alternatively, the tablet may be coated with a concentrated sugar solution which may contain, for example, gum arabic, gelatine, talcum and titanium dioxide.

For the preparation of soft gelatine capsules, the compound of the disclosure may be admixed with, for example, a vegetable oil or polyethylene glycol. Hard gelatine capsules may contain granules of the compound using pharmaceutical excipients like the abovementioned excipients for tablets. Additionally, liquid or semisolid formulations of the compound of the disclosure may be filled into hard gelatine capsules.

Liquid preparations for oral application may be in the form of syrups, solutions or suspensions. Solutions, for example may contain the compound of the disclosure, the balance being sugar and a mixture of ethanol, water, glycerol and propylene glycol. Optionally such liquid preparations may contain coloring agents, flavoring agents, saccharine and/or carboxymethylcellulose as a thickening agent. Furthermore, other excipients known to those skilled in art may be used when making formulations for oral use.

### Therapeutic Use

In embodiments, the compounds of Formula (I), (II), (III), (IV), (V), (VI), (VII), (VIII), (IX), (X), (XI), (XII), (XIII), (XIV), (XV), (XVI), (XVII), (XVIII), (XIX) or (XX), or Table 1 and their pharmaceutically acceptable salts, are DPP1 inhibitors, and thus may be used in any disease area where DPP1 plays a role. As such, in one aspect of the invention, a method of treatment is provided. The method of treatment, in one embodiment, comprises, administering to a subject in need thereof, a composition comprising an effective amount of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX), or Table 1 or a pharmaceutically acceptable salt of Formula (I), (II), (III), (IV), (V), (VI), (VII), (VIII), (IX), (X), (XI), (XII), (XIII), (XIV), (XV), (XVI), (XVII), (XVIII), (XIX) or (XX), or Table 1. In embodiments, the composition is administered to the patient for an administration period.

In embodiments, a compound or composition of the present disclosure is administered to a patient in a method for treating a obstructive disease of the airway; chronic rhinosinusitis (CRS); hidradenitis suppurativa (HS); cancer (e.g., cancer metastasis); granulomatosis with polyangiitis (GPA); microscopic polyangiitis (MPA); giant cell arteritis; polyarteritis nodosa; anti-GBM disease (Goodpasture's); rheumatoid arthritis; lupus nephritis; systemic lupus erythematosus; systemic scleroderma; inflammatory bowel disease (IBD) (e.g., ulcerative colitis; Crohn's disease); diabetic nephropathy; diabetic neuropathy; diabetic retinopathy; diabetic ulcers; Duchenne muscular dystrophy; bronchiolitis obliterans; long covid)—prophylaxis of ILD; atopic dermatitis; pyoderma gangrenosum; sweet's syndrome; dermatomyositis/polymyositis; neutrophilic dermatoses; uveitis; Behcet's disease; thrombosis; bronchopulmonary dysplasia; amyotrophic lateral sclerosis; sickle cell anemia; psoriasis; ventilator-induced lung injury.

In embodiments, a compound or composition of the present disclosure is administered to a patient in a method for treating an obstructive disease of the airway. The obstructive disease of the airway, in one embodiment, is asthma (e.g., bronchial, allergic, intrinsic, extrinsic, exercise-induced, drug-induced (including aspirin and NSAID-induced and dust-induced asthma, both intermittent and persistent and of all severities) airway hyper-responsiveness, chronic obstructive pulmonary disease (COPD), bronchitis (e.g., infectious bronchitis, eosinophilic bronchitis), emphysema, cystic fibrosis (CF), bronchiectasis (e.g., non-CF bronchiectasis (NCFBE) and bronchiectasis associated with CF), cystic fibrosis; sarcoidosis; alpha-1 antitrypsin (A1AT) deficiency, farmer's lung and related diseases, hypersensitivity pneumonitis, interstitial lung disease, lung fibrosis (including idiopathic pulmonary fibrosis, cryptogenic fibrosing alveolitis, idiopathic interstitial pneumonias, fibrosis complicating anti-neoplastic therapy and chronic infection, including tuberculosis and aspergillosis and other fungal infections), complications of lung transplantation, vasculitic and thrombotic disorders of the lung vasculature, pulmonary hypertension (e.g., pulmonary arterial hypertension), antitussive activity including treatment of chronic cough associated with inflammatory and secretory conditions of the airways, iatrogenic cough, acute and chronic rhinitis including rhinitis medicamentosa, and vasomotor rhinitis; perennial and seasonal allergic rhinitis including rhinitis nervosa (hay fever), nasal polyposis; acute viral infection including the common cold, and infection due to a respiratory virus (e.g., respiratory syncytial virus, influenza, coronavirus (including SARS) and adenovirus), acute lung injury, acute respiratory distress syndrome (ARDS), as well as exacerbations of each of the foregoing respiratory tract disease states.

Cystic fibrosis (CF) is caused by abnormalities in the CF transmembrane conductance regulator protein, causing chronic lung infections (particularly with Pseudomonas aeruginosa) and excessive inflammation, and leading to bronchiectasis, declining lung function, respiratory insufficiency and quality of life. The inflammatory process is dominated by neutrophils that produce NE, as well as other destructive NSPs including CatG and PR3, that directly act upon extracellular matrix proteins and play a role in the host response to inflammation and infection (Dittrich et al., Eur Respir J. 2018; 51(3)). The methods provided herein employ reversible inhibitors of DPP1. Without wishing to be bound by theory, it is thought that the compounds of Formula (I), (II), or (III), administered via the methods provided herein have beneficial effects via inhibiting the activation of NSPs and decreasing inflammation, which in turn leads to a decrease in pulmonary exacerbations, a decrease in the rate of pulmonary exacerbations, and/or an improvement in lung function (e.g., forced expiratory volume in 1 second [FEV₁]) in CF patients.

In one embodiment, a method is provided for treating CF comprising administering to a CF patient in need of treatment, a composition comprising an effective amount of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX), or a pharmaceutically acceptable salt thereof, or a deuterated form thereof.

In one CF treatment method, a composition comprising an effective amount of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX), or a pharmaceutically acceptable salt thereof, or a deuterated form thereof, is administered to a CF patient in need of treatment for an administration period. The method comprises improving the lung function of the patient during the administration period, as compared to the lung function of the patient prior to the administration period. The improvement in lung function in one embodiment, is measured by spirometry.

Improving the lung function of the patient, in one embodiment, comprises increasing the patient's forced expiratory volume in 1 second (FEV₁), increasing the patient's forced vital capacity (FVC), increasing the patient's peak expiratory flow rate (PEFR), or increasing the patient's forced expiratory flow between 25% and 75% of FVC (FEF_{(25-75%)}), as compared to the respective value prior to the administration period. Increasing, in one embodiment, is by about 5%, by about 10%, by about 15%, by about 20%, by about 25%, by about 30%, by about 35%, by about 40%, by about 45% or by about 50% of the respective value. Increasing, in one embodiment, is by at least about 5%, by at least about 10%, by at least about 15%, by at least about 20%, by at least about 25%, by at least about 30%, by at least about 35%, by at least about 40%, by at least about 45% or by at least about 50%. In yet another embodiment, the increasing is by about 5% to about 50%, by about 5% to about 40%, by about 5% to about 30% or by about 5% to about 20%. In even another embodiment, increasing is by about 10% to about 50%, by about 15% to about 50%, by about 20% to about 50%, or by about 25% to about 50%.

In one embodiment of a method provided herein, a composition comprising an effective amount of a compound of Formula (I), (II), (III), (IV), (V), (VI), (VII), (VIII), (IX), (X), (XI), (XII), (XIII), (XIV), (XV), (XVI), (XVII), (XVIII), (XIX) or (XX), or a pharmaceutically acceptable salt thereof, or a deuterated form thereof, is administered to a bronchiectasis patient in need of treatment for an administration period. Bronchiectasis is considered a pathological endpoint that results from many disease processes and is a persistent or progressive condition characterized by dilated thick-walled bronchi. The symptoms vary from intermittent episodes of expectoration and infection localized to the region of the lung that is affected to persistent daily expectoration often of large volumes of purulent sputum. Bronchiectasis may be associated with other non-specific respiratory symptoms. The underlying pathological process of bronchiectasis, without wishing to be bound by theory, has been reported as damage to the airways which results from an event or series of events where inflammation is central to the process (Guideline for non-CF Bronchiectasis, Thorax, July 2010, V. 65(Suppl 1), incorporated by reference herein in its entirety for all purposes).

Bronchiectasis is considered a pathological endpoint that results from many disease processes and is a persistent or progressive condition characterized by dilated thick-walled bronchi. The symptoms vary from intermittent episodes of expectoration and infection localized to the region of the lung that is affected to persistent daily expectoration often of large volumes of purulent sputum. Bronchiectasis may be associated with other non-specific respiratory symptoms. The underlying pathological process of bronchiectasis, without wishing to be bound by theory, has been reported as damage to the airways which results from an event or series of events where inflammation is central to the process (Guideline for non-CF Bronchiectasis, Thorax, July 2010, V. 65(Suppl 1), incorporated by reference herein in its entirety for all purposes).

The methods provided herein employ reversible inhibitors of DPP1. Without wishing to be bound by theory, it is thought that the compounds of Formula (I), (II), or (III), administered via the methods provided herein have beneficial effects via decreasing inflammation and mucus hypersecretion, which in some embodiments, leads to a decrease in pulmonary exacerbations, a decrease in the rate of pulmonary exacerbations, and/or an improvement in lung function (cough, sputum production, and forced expiratory volume in 1 second [FEV₁]) in bronchiectasis patients. Without wishing to be bound by theory, it is thought that the methods provided herein modify bronchiectasis progression by reducing the accelerated rate of lung function decline or lung tissue destruction.

In one embodiment, the bronchiectasis is non-CF bronchiectasis.

In one embodiment, the method for treating bronchiectasis comprises improving lung function of the patient during the administration period, as compared to the lung function of the patient prior to the administration period.

A pulmonary exacerbation, in one embodiment, is characterized by three or more of the following symptoms exhibited for at least 48 hours by the patient: (1) increased cough; (2) increased sputum volume or change in sputum consistency; (3) increased sputum purulence; (4) increased breathlessness and/or decreased exercise tolerance; (5) fatigue and/or malaise; (6) hemoptysis. In a further embodiment, the three or more symptoms result in a physician's decision to prescribe an antibiotic(s) to the patient exhibiting the symptoms.

In one embodiment of a method for treating bronchiectasis, the method comprises decreasing the rate of pulmonary exacerbation in the subject, compared to the rate of pulmonary exacerbation experienced by the subject prior to the administration period of the composition, or compared to a control subject with bronchiectasis that is not subject to the method of treatment. In a further embodiment, the bronchiectasis is non-CF bronchiectasis.

In another aspect, a method for treating chronic rhinosinusitis (CRS) in a subject in need thereof is provided. The method comprises in one embodiment, administering to the subject for an administration period, a pharmaceutical composition comprising an effective amount of a compound of Formula (I), (II), or (III), or a pharmaceutically acceptable salt thereof, or a deuterated form thereof.

The chronic rhinosinusitis is chronic rhinosinusitis without nasal polyps (CRSsNP), or chronic rhinosinusitis with nasal polyps (CRSwNP). In some embodiments, the chronic rhinosinusitis is chronic rhinosinusitis without nasal polyps (CRSsNP). In some embodiments, the chronic rhinosinusitis is chronic rhinosinusitis with nasal polyps (CRSwNP). In some embodiments, the chronic rhinosinusitis is refractory chronic rhinosinusitis. In some embodiments, the refractory chronic rhinosinusitis is refractory chronic rhinosinusitis without nasal polyps (CRSsNP). In some embodiments, the refractory chronic rhinosinusitis is refractory chronic rhinosinusitis with nasal polyps (CRSwNP).

In some embodiments, the subject exhibits one or more symptoms of CRS. In some embodiments, the one or more symptoms of CRS are: (a) nasal congestion; (b) nasal obstruction; (c) nasal discharge; (d) post-nasal drip; (e) facial pressure; (f) facial pain; (g) facial fullness; (h) reduced smell; (i) depression; (j) mucosal edema; (k) mucopurulent discharge; (1) obstruction of the middle meatus; (m) mucosal changes within the ostiomeatal complex and sinuses; (n) rhinorrhea; or (o) any combinations thereof. In some embodiments, obstruction of the middle meatus is mucosal obstruction, edematous obstruction, or a combination thereof.

In some embodiments, the administration of the pharmaceutical composition reduces, diminishes the severity of, delays the onset of, or eliminates one or more symptoms of CRS. In some embodiments, the one or more symptoms of CRS are: (a) nasal congestion; (b) nasal obstruction; (c) nasal discharge; (d) post-nasal drip; (e) facial pressure; (f) facial pain; (g) facial fullness; (h) reduced smell; (i) depression; (j) mucosal edema; (k) mucopurulent discharge; (l) obstruction of the middle meatus; (m) mucosal changes within the ostiomeatal complex and sinuses; (n) rhinorrhea; (o) or any combinations thereof. In some embodiments, the administration of the pharmaceutical composition enhances sinus drainage.

In some embodiments, the methods comprise reducing a composite severity score of one or more symptoms of CRS. As used herein, the “composite severity score” is a quantitative measure of all the symptoms of CRS exhibited by the subject. In some embodiments, the composite severity score is a sum total of all the daily symptoms exhibited by the subject. In some embodiments, the composite severity score is reduced during or subsequent to the administration period, as compared to the composite severity score measured prior to the administration period. In some embodiments, the one or more symptoms of CRS exhibited by the subject may be any symptoms described herein or known in the art to be associated with CRS. In some embodiments, the one or more symptoms of CRS are: nasal congestion, reduced smell, rhinorrhea, or any combination thereof. In some embodiments, the rhinorrhea is anterior rhinorrhea. In some embodiments, the rhinorrhea is posterior rhinorrhea.

In some embodiments, the methods comprise decreasing the Sino-Nasal Outcome Test-22 (SNOT-22) score of the subject during the administration period or subsequent to the administration period, compared to the SNOT-22 score of the subject prior to the administration period. As used herein, “SNOT-22” is a patient-reported measure of outcome developed for use in CRS with or without nasal polyps and contains 22 individual questions. The questions cover a broad range of health and health-related quality of life problems including physical problems, functional limitations and emotional consequences. The theoretical range of the SNOT-22 score is 0-110, with lower scores implying a better health-related quality of life. Further details of SNOT-22 are provided in Hopkins, et al., Clin. Otolaryngol. 2009, 34, 447-454, and Kennedy, et al., Ann Allergy Asthma Immunol. 2013 October; 111(4): 246-251, the contents of which are incorporated herein by reference in its entirety.

Hidradenitis suppurativa (HS) is a chronic relapsing inflammatory disorder. The symptoms include skin lesions that are often associated hair follicles, and may be painful, inflamed and/or swollen. In some cases, when the skin lesions heal, they can recur, and may lead to tunnels under the skin and progressive scarring. Since HS is a chronic condition, it can persist for many years and also, worsen over time, with serious effects on quality of life, psychological and emotional well-being. In fact, HS patients have increased rates of anxiety and depression with a risk of suicide two and a half times that of the general population.

HS patients are categorized according to disease severity, termed Hurley staging, as mild (Stage I), moderate (Stage II), or severe (Stage III). Although more than 200,000 cases of HS are diagnosed in the U.S. per year, this disease can be difficult to diagnose and requires specialized care. HS may be mistaken for an infection, an ingrown hair or other conditions. Moreover, current treatment options are limited and lack efficacy.

In one aspect, a method of treating HS in a subject in need thereof is provided. The method comprises in one embodiment, administering to the subject for an administration period, a pharmaceutical composition comprising an effective amount of a compound of Formula (I), (II), or (III), or a pharmaceutically acceptable salt thereof, or a deuterated form thereof. In a further embodiment, the method of treating HS comprises reducing neutrophilic inflammation in the subject.

The HS in one embodiment, is Hurley Stage I HS, Hurley Stage II HS or Hurley Stage III HS. In some embodiments, the HS is Hurley Stage I HS. In some embodiments, the HS is Hurley Stage II HS. In some embodiments, the HS is Hurley Stage III HS.

The disclosure provides methods of treating cancer in a subject in need thereof, comprising, administering to the subject, a pharmaceutical composition comprising an effective amount of any one of the compounds disclosed herein. The disclosure provides methods of treating cancer-induced pain in a subject having cancer, comprising, administering to the subject for an administration period, a pharmaceutical composition comprising an effective amount of any one of the compounds disclosed herein. In some embodiments, the cancer-induced pain is cancer-induced bone pain. The disclosure also provides methods of treating cancer-induced bone pain in a subject having cancer, comprising, administering to the subject for an administration period, a pharmaceutical composition comprising an effective amount of any one of the compounds disclosed herein.

In some embodiments, the cancer comprises a primary solid tumor. In some embodiments, the cancer is bladder cancer, lung cancer, brain cancer, ovarian cancer, pancreatic cancer, colorectal cancer, prostate cancer, liver cancer, hepatocellular carcinoma, kidney cancer, stomach cancer, skin cancer, fibroid cancer, lymphoma, virus-induced cancer, oropharyngeal cancer, testicular cancer, thymus cancer, thyroid cancer, melanoma, or bone cancer.

In some embodiments, the cancer is bladder cancer. In some embodiments, the cancer is lung cancer. In some embodiments, the cancer is brain cancer. In some embodiments, the cancer is ovarian cancer. In some embodiments, the cancer is pancreatic cancer. In some embodiments, the cancer is colorectal cancer. In some embodiments, the cancer is prostate cancer. In some embodiments, the cancer is liver cancer. In some embodiments, the cancer is hepatocellular carcinoma. In some embodiments, the cancer is kidney cancer. In some embodiments, the cancer is stomach cancer. In some embodiments, the cancer is skin cancer. In some embodiments, the cancer is fibroid cancer. In some embodiments, the cancer is lymphoma. In some embodiments, the cancer is virus-induced cancer. In some embodiments, the cancer is oropharyngeal cancer. In some embodiments, the cancer is testicular cancer. In some embodiments, the cancer is thymus cancer. In some embodiments, the cancer is thyroid cancer. In some embodiments, the cancer is melanoma. In some embodiments, the cancer is bone cancer. In some embodiments, the fibroid cancer is leiomyosarcoma.

In some embodiments, the breast cancer comprises ductal carcinoma, lobular carcinoma, medullary carcinoma, colloid carcinoma, tubular carcinoma, or inflammatory breast cancer. In some embodiments, the breast cancer comprises ductal carcinoma. In some embodiments, the breast cancer comprises lobular carcinoma. In some embodiments, the breast cancer comprises medullary carcinoma. In some embodiments, the breast cancer comprises colloid carcinoma. In some embodiments, the breast cancer comprises tubular carcinoma. In some embodiments, the breast cancer comprises inflammatory breast cancer.

In some embodiments, the breast cancer is triple-negative breast cancer. In some embodiments, the breast cancer does not respond to hormonal therapy or therapeutics that target the HER2 protein receptors.

In some embodiments, the lymphoma is Hodgkin's lymphoma, non-Hodgkin's lymphoma, diffuse large B-cell lymphoma, B-cell immunoblastic lymphoma, Natural Killer cell lymphoma, T-cell lymphoma, Burkitt lymphoma or Kaposi's Sarcoma. In some embodiments, the lymphoma is Hodgkin's lymphoma. In some embodiments, the lymphoma is non-Hodgkin's lymphoma. In some embodiments, the lymphoma is diffuse large B-cell lymphoma. In some embodiments, the lymphoma is B-cell immunoblastic lymphoma. In some embodiments, the lymphoma is Natural Killer cell lymphoma. In some embodiments, the lymphoma is T-cell lymphoma. In some embodiments, the lymphoma is Burkitt lymphoma. In some embodiments, the lymphoma is Kaposi's Sarcoma.

In some embodiments, the brain cancer is astrocytoma, anaplastic astrocytoma, glioblastoma multiforme, oligodendroglioma, ependymoma, meningioma, schwannoma, or medulloblastoma. In some embodiments, the brain cancer is astrocytoma. In some embodiments, the brain cancer is anaplastic astrocytoma. In some embodiments, the brain cancer is glioblastoma multiforme. In some embodiments, the brain cancer is oligodendroglioma. In some embodiments, the brain cancer is ependymoma. In some embodiments, the brain cancer is meningioma. In some embodiments, the brain cancer is schwannoma. In some embodiments, the brain cancer is medulloblastoma.

In some embodiments, the cancer is liquid tumor. In some embodiments, the liquid tumor is acute myeloid leukemia (AML), acute lymphoblastic leukemia, acute lymphocytic leukemia, acute promyelocytic leukemia, chronic myeloid leukemia, hairy cell leukemia, a myeloproliferative disorder, Natural Killer cell leukemia, blastic plasmacytoid dendritic cell neoplasm, chronic myelogenous leukemia (CML), mastocytosis, chronic lymphocytic leukemia (CLL), multiple myeloma (MM), or myelodysplastic syndrome (MDS). In some embodiments, the liquid tumor is acute myeloid leukemia (AML). In some embodiments, the liquid tumor is acute lymphoblastic leukemia. In some embodiments, the liquid tumor is acute lymphocytic leukemia. In some embodiments, the liquid tumor is acute promyelocytic leukemia. In some embodiments, the liquid tumor is chronic myeloid leukemia. In some embodiments, the liquid tumor is hairy cell leukemia. In some embodiments, the liquid tumor is a myeloproliferative disorder. In some embodiments, the liquid tumor is Natural Killer cell leukemia. In some embodiments, the liquid tumor is blastic plasmacytoid dendritic cell neoplasm. In some embodiments, the liquid tumor is chronic myelogenous leukemia (CML). In some embodiments, the liquid tumor is mastocytosis. In some embodiments, the liquid tumor is chronic lymphocytic leukemia (CLL). In some embodiments, the liquid tumor is multiple myeloma (MM). In some embodiments, the liquid tumor is myelodysplastic syndrome (MDS).

In some embodiments, the cancer is a pediatric cancer. In some embodiments, the pediatric cancer is neuroblastoma, Wilms tumor, rhabdomyosarcoma, retinoblastoma, osteosarcoma or Ewing sarcoma. In some embodiments, the pediatric cancer is neuroblastoma. In some embodiments, the pediatric cancer is Wilms tumor. In some embodiments, the pediatric cancer is rhabdomyosarcoma. In some embodiments, the pediatric cancer is retinoblastoma. In some embodiments, the pediatric cancer is osteosarcoma. In some embodiments, the pediatric cancer is Ewing sarcoma.

In some embodiments, the cancer is metastatic cancer. In some embodiments, the subject is at a risk for developing metastatic cancer. In some embodiments, the metastatic cancer comprises metastasis of breast cancer to the brain, bone, pancreas, lymph nodes, and/or liver. In some embodiments, the metastatic cancer comprises metastasis of bone cancer to the lung. In some embodiments, the metastatic cancer comprises metastasis of colorectal cancer to the peritoneum, the pancreas, the stomach, the lung, the liver, the kidney, and/or the spleen. In some embodiments, the metastatic cancer comprises metastasis of stomach cancer to the mesentery, the spleen, the pancreas, the lung, the liver, the adrenal gland, and/or the ovary. In some embodiments, the metastatic cancer comprises metastasis of leukemia to the lymph nodes, the lung, the liver, the hind limb, the brain, the kidney, and/or the spleen. In some embodiments, the metastatic cancer comprises metastasis of liver cancer to the intestine, the spleen, the pancreas, the stomach, the lung, and/or the kidney. In some embodiments, the metastatic cancer comprises metastasis of lymphoma to the kidney, the ovary, the liver, the bladder, and/or the spleen.

In some embodiments, the metastatic cancer comprises metastasis of hematopoietic cancer to the intestine, the lung, the liver, the spleen, the kidney, and/or the stomach. In some embodiments, the metastatic cancer comprises metastasis of melanoma to lymph nodes and/or the lung. In some embodiments, the metastatic cancer comprises metastasis of pancreatic cancer to the mesentery, the ovary, the kidney, the spleen, the lymph nodes, the stomach, and/or the liver. In some embodiments, the metastatic cancer comprises metastasis of prostate cancer to the lung, the pancreas, the kidney, the spleen, the intestine, the liver, the bone, and/or the lymph nodes. In some embodiments, the metastatic cancer comprises metastasis of ovarian cancer to the diaphragm, the liver, the intestine, the stomach, the lung, the pancreas, the spleen, the kidney, the lymph nodes, and/or the uterus. In some embodiments, the metastatic cancer comprises metastasis of myeloma to the bone.

In some embodiments, the metastatic cancer comprises metastasis of lung cancer to the bone, the brain, the lymph nodes, the liver, the ovary, and/or the intestine. In some embodiments, the metastatic cancer comprises metastasis of kidney cancer to the liver, the lung, the pancreas, the stomach, the brain, and/or the spleen. In some embodiments, the metastatic cancer comprises metastasis of bladder cancer to the bone, the liver and/or the lung. In some embodiments, the metastatic cancer comprises metastasis of thyroid cancer to the bone, the liver and/or the lung.

In some embodiments, the methods disclosed herein comprise treating cancer-induced bone pain (CIBP) in a subject having metastasis of a cancer to the bone. In some embodiments, the subject has metastasis of prostate cancer, breast cancer, lung cancer, or myeloma to the bone. In some embodiments, the subject is identified as having metastasis to the bone by the use of any one of the following methods: plain film radiography, computed tomography, technetium 99m bone scan, magnetic resonance imaging, fluorodeoxyglucose positron emission tomography, fluorine positron emission tomography, and/or choline positron emission tomography, but is not yet feeling cancer-induced bone pain. In some embodiments, the subject is suffering from cancer-induced bone pain, which is indicative of metastasis of a previously treated or untreated primary tumor to the bone. In some embodiments, the cancer has metastasized to vertebrae, pelvis, long bones, or ribs.

In some embodiments, administration of the composition diminishes the severity of, delays the onset of, or eliminates a symptom of cancer. In some embodiments, the symptom of cancer is cancer-induced bone pain (CIBP). In some embodiments, the CIBP is neuropathic pain. In some embodiments, the CIBP is inflammatory pain. In some embodiments, the CIBP is spontaneous pain. In some embodiments, the symptom of cancer is nociceptive hypersensitivity. In some embodiments, the symptom of cancer is allodynia. In some embodiments, the allodynia is tactile allodynia. In some embodiments, the tactile allodynia is static mechanical allodynia. In some embodiments, the tactile allodynia is dynamic mechanical allodynia. In some embodiments, the subject has bone cancer or metastasis to the bone.

In yet another embodiment of the present invention, a method for treating lupus nephritis (LN) in a subject in need thereof is provided. The method comprises administering to the subject for an administration period, a pharmaceutical composition comprising an effective amount of a compound of Formula (I), (II), or (III), or a pharmaceutically acceptable salt thereof, or a deuterated form thereof.

Rheumatoid arthritis (RA) is characterized by inflammation and thickening of the joint capsule, together with an effect on the underlying bone and cartilage. Currently, the cause of RA is unknown and no satisfactory cure for RA is available. While a number of therapeutic agents have been developed and utilized to alleviate pain and inflammation associated with the disease, such as disease-modifying antirheumatic drugs (DMARDs) and non-steroidal anti-inflammatory agents (NSAIDs), they often produce intolerable side effects. To addresses this and other needs, the present invention, in one embodiment, provides a method for treating RA using reversible inhibitors of DPP1 of Formula (I), (II), or (III). In one embodiment, a method of for treating RA in a subject in need thereof is provided, and comprises administering to the subject for an administration period, a pharmaceutical composition comprising an effective amount of a compound of Formula (I), (II), or (III), or a pharmaceutically acceptable salt thereof, or a deuterated form thereof. In a further embodiment, the method comprises reducing neutrophilic inflammation in the subject.

Inflammatory bowel disease (IBD) is a group of inflammatory conditions that affect the colon and small intestine. The most common IBDs are Crohn's disease and ulcerative colitis. The present invention, in one embodiment, addresses the need for novel IBD therapies. Specifically, in one embodiment, a method for treating an inflammatory bowel disease (IBD) in a subject in need thereof is provided. The method comprises administering to the subject for an administration period, a pharmaceutical composition comprising an effective amount of a compound of Formula (I), or a pharmaceutically acceptable salt thereof, or a deuterated form thereof.

In a further embodiment, the IBD is Crohn's disease or ulcerative colitis. In even a further embodiment, the method comprises reducing neutrophilic inflammation in the subject.

The length of the administration period in any given case may depend on the nature and severity of the condition being treated and/or prevented and be determined by the physician. In one embodiment, the administration period starts at about the time of condition/disease diagnosis and continues for the lifetime of the patient.

### EXAMPLES

The present invention is further illustrated by reference to the following Examples. However, it should be noted that these Examples, like the embodiments described above, are illustrative and are not to be construed as restricting the scope of the invention in any way.

In embodiments, compounds of the present invention can be synthesized using the following methods. General reaction conditions are given, and reaction products can be purified by generally known methods including silica gel chromatography using various organic solvents such as hexane, dichloromethane, ethyl acetate, methanol and the like or preparative reverse phase high pressure liquid chromatography.

In the following examples, the term “assumed”, where present, refers to the particular stereochemistry of the respective product. Further characterization will confirm the absolute stereochemistry of the products.

### General Experimental

### ¹H NMR Analysis:

¹H-NMR spectra are recorded on a Bruker Ultrashield (400 MHz). The multiplicity of a signal is designated by the following abbreviations: s, singlet; d, doublet; t, triplet; q, quartet; dd, doublet of doublets; dt, doublet of triplets; m, multiplet.

All observed coupling constants, J, are reported in Hertz (Hz).

Exchangeable protons are not always observed.

### Lc/MS Analysis:

### LC/MS Method AN01_001_012:

LC-MS data was generated using a Waters Acquity system: TUV detector, SQD2 MS detector, Sedere SEDEX 80 (light scattering detector).
LC-MS method: reverse phase HPLC analysis
Column Agilent: Cortecs C18
Solvent A: Water with Formic Acid (0.1% V/V)
Solvent B: Acetonitrile

Gradient table:

| Time | Flow | | | |
|---|---|---|---|---|
| (min) | (mL/min) | % A | % B | Curve |
| 0 | 0.7 | 98 | 2 | initial |
| 2 | 0.7 | 0 | 100 | Linear |
| 2.5 | 0.7 | 0 | 100 | Linear |
| 2.6 | 0.7 | 98 | 2 | Linear |
| 5 | 0.7 | 98 | 2 | Linear |

### LC/MS Method AN01_001_026:

LC-MS data was generated using a Waters Acquity system: TUV detector, SQD2 MS detector, Sedere SEDEX 80 (light scattering detector).
LC-MS method: reverse phase HPLC analysis
Column Agilent: Poroshell
Solvent A: Water with Formic Acid (0.1% V/V)
Solvent B: Acetonitrile

Gradient table:

| Time | Flow | | | |
|---|---|---|---|---|
| (min) | (mL/min) | % A | % B | Curve |
| 0 | 1 | 98 | 2 | initial |
| 2 | 1 | 98 | 2 | 6 |
| 12 | 1 | 0 | 100 | 6 |
| 15.4 | 1 | 0 | 100 | 6 |
| 16.7 | 1 | 98 | 2 | 6 |
| 18.4 | 1 | 98 | 2 | 6 |

UV detection: 220 nm

### General Synthetic Procedures

### Procedure A

To an argon-purged solution of protected alcohol (1 eq.) in EtOH (4.78 mL/mmol of protected alcohol) is added 10% Pd/C (0.1 eq.) at room temperature. The resulting mixture is purged with argon (×3) and then with H₂ (3×). The reaction mixture is stirred under an atmospheric pressure of H₂ at room temperature for 18 h. The reaction mixture is purged with argon, filtered on a pad of celite and rinsed with EtOH (3×5 mL). The filtrate is concentrated under reduced pressure to afford the expected compound.

### Procedure B

To a solution of alcohol derivative (1 eq.) in acetone (16.7 mL/mmol of alcohol) and sodium bromide (0.3 eq.) is added a saturated aqueous solution of NaHCO₃ (2.59 mL/mmol of alcohol) at room temperature. To the resulting mixture are added trichlorocyanuric acid (2.2 eq.) and 2,2,6,6-tetramethylpiperidine-1-oxyl (0.03 eq.) at 0° C. The reaction mixture is allowed to warm to room temperature and stirred for 18 h. Isopropanol (10 mL) is added at room temperature and the reaction mixture is stirred for 30 min. The reaction mixture is diluted with EtOAc (50 mL) and a saturated aqueous solution of NaHCO₃ (50 mL) is added. The two layers are separated, and the aqueous layer is washed with EtOAc (50 mL). The aqueous layer is then acidified with an aqueous solution of 3M HCl until pH~1 and extracted with DCM (2×50 mL). The combined organic layers are dried over Na₂SO₄, filtered and concentrated under reduced pressure to afford the expected compound.

### Procedure C

To a solution of amine derivative (1 eq.) in anhydrous DMF (7.14 mL/mmol of amine) and carboxylic acid derivative (1.05 eq.) are added DIPEA (2.5 eq.) and TBTU (1.5 eq.) at room temperature under argon atmosphere. The reaction mixture is stirred at room temperature for 18 h. The reaction mixture is diluted with EtOAc (10 mL) and water (10 mL). The aqueous layer is extracted with EtOAc (2×10 mL) and the combined organic layers are washed with brine (3×10 mL), dried over Na₂SO₄, filtered and concentrated under reduced pressure. The crude is purified by flash chromatography over SiO₂ (see conditions for each compound) to afford the expected compound.

### Procedure D

To a preheated vial (50° C.) containing Boc protected amine derivative (1 eq.) is added formic acid (7.6 mL/mmol) also preheated at 50° C. The reaction mixture is stirred at 50° C. for 15 min. The reaction mixture is cooled back to room temperature and added dropwise into a cooled (0° C.) mixture of stirred aqueous solution of saturated NaHCO₃ (40 mL) and DCM (40 mL). The layers are separated, and the aqueous layer is extracted with DCM (2×40 mL). The combined organic layers are dried over Na₂SO₄, filtered and concentrated to under reduced pressure. The crude is purified by flash chromatography over SiO₂ and/or preparative HPLC (see conditions for each compound) to afford the expected compound.

### Procedure E

To a solution of alcohol derivative (1 eq.) in anhydrous DMF (5.49 mL/mmol of alcohol derivative) and iodomethane (2 eq.) is added NaH 60% in oil (1.1 eq.) at 0° C. under argon atmosphere. The resulting mixture is allowed to warm to room temperature and stirred for 22 h. The reaction mixture is quenched with a saturated aqueous solution of NH₄C₁ (10 mL) at room temperature. EtOAc (50 mL) and water (50 mL) are then added and the two layers are separated. The aqueous layer is extracted with EtOAc (2×50 mL) and the combined organic layers are dried over Na₂SO₄, filtered and concentrated under reduced pressure. The crude residue is purified by silica gel flash chromatography (see conditions for each compound) to afford the expected compound.

### Example 1: Preparation of B1-46-1-(S)* & B1-46-1-(R)*

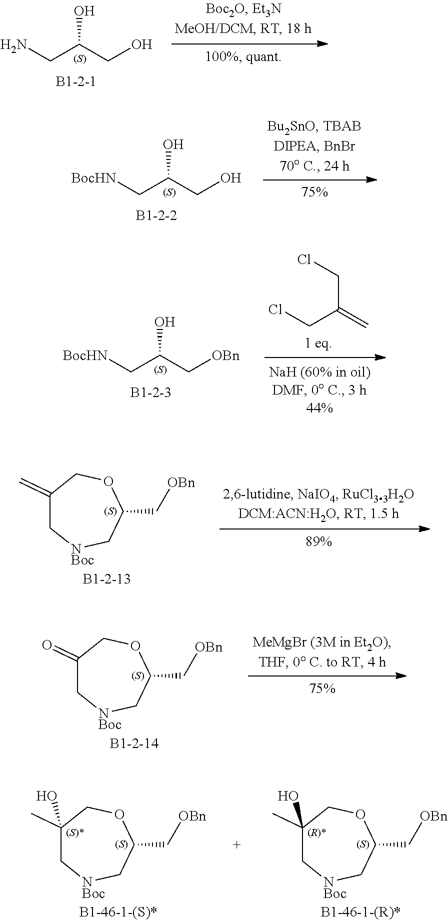

### tert-butyl N-[(2S)-2,3-dihydroxypropyl]carbamate (B1-2-2)

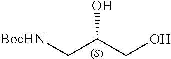

To a solution of (2S)-3-aminopropane-1,2-diol (1 eq., 14.4 g, 158.1 mmol) and Et₃N (1.01 eq., 22.2 mL, 159.6 mmol) in anhydrous MeOH (245 mL) was added a solution of Boc₂O (1.2 eq., 41.4 g, 189.7 mmol) in anhydrous DCM (41 mL) at room temperature under argon atmosphere. The reaction mixture was stirred at room temperature for 18 h and then concentrated under reduced pressure to afford B1-2-2 as a pale yellow oil (30.2 g, quant.). The crude was considered quantitative and used as such.

LC/MS (ANO 1_001_012): Rt=1.69 min, non-UV active, [M+Na]⁺=214.1.

### tert-butyl N-[(2S)-3-(benzyloxy)-2-hydroxypropyl]carbamate (B1-2-3)

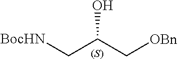

To a solution of B1-2-2 (1 eq., 1.72 g, 8.99 mmol), di(n-butyl)tin oxide (0.1 eq., 0.224 g, 0.899 mmol) and TBAB (0.3 eq., 0.870 g, 2.70 mmol) were added DIPEA (2 eq., 3.13 mL, 18.0 mmol) and BnBr (2 eq., 2.15 mL, 18.0 mmol) at room temperature under argon atmosphere. The reaction mixture was stirred at 70° C. for 6 h. The reaction mixture was concentrated under reduced pressure then taken into EtOAc (50 mL) and filtered over a pad of silica gel. The latter was rinsed with EtOAc (3×150 mL) and the filtrate was concentrated under reduced pressure. The resulting orange oil (4.46 g) was purified by silica gel flash chromatography (120 g, gradient: cyclohexane/EtOAc 100:0 to 50:50) to afford B1-2-3 as a pale-yellow oil (2.31 g, 75%) which was contaminated by the other -OBn regioisomer protected in position 2 (17 wt % by ¹H NMR analysis).

LC/MS (AN01_001_012): Rt=2.28 min, 100%, [M+Na]+=304.1.

### tert-butyl (2S)-2-[(benzyloxy)methyl]-6-methylidene-1,4-oxazepane-4-carboxylate B1-2-13

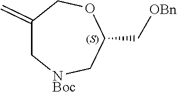

To a suspension of NaH 60% in oil (2.1 eq., 3.40 g, 85.1 mmol) in anhydrous DMF (72 mL) was added 3-chloro-2-chloromethyl-1-propene (1 eq., 4.69 mL, 40.5 mmol) at 0° C. The reaction mixture was stirred at 0° C. for 10 min, then a solution of B1-2-3 (1 eq., 11.4 g, 40.5 mmol) in anhydrous THE (50 mL) was added dropwise at 0° C. The reaction mixture was stirred at room temperature for 4 h. The reaction mixture was diluted with water (200 mL) and the aqueous layer was extracted with EtOAc (3×200 mL). The combined organic layers were washed with brine (200 mL), dried over Na₂SO₄, filtered and concentrated under reduced pressure. The crude residue was purified by silica gel flash chromatography (330 g, gradient: Cyclohexane/EtOAc from 100:0 to 90:10) to afford B1-2-13 as a colorless oil (5.93 g, 44%).

LC/MS (ANO 1_001_012): Rt=2.73 min, 100%, [M-C₄H₈+H]⁺=278.1.

### tert-butyl (2S)-2-[(benzyloxy)methyl]-6-oxo-1,4-oxazepane-4-carboxylate B1-2-14

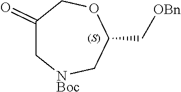

To a solution of B1-2-13 (1 eq., 2.10 g, 6.30 mmol) in a mixture of DCM (38 mL) and acetonitrile (38 mL) were added 2,6-lutidine (2 eq., 1.47 mL, 12.6 mmol), water (57 mL) and sodium periodate (4 eq., 5.39 g, 25.2 mmol) at room temperature. A solution of RuCl₃·3H₂O (0.035 eq., 57.6 mg, 0.220 mmol) in water (6.3 mL) was added dropwise forming a brown suspension. The reaction mixture was vigorously stirred at room temperature for 2 h. The reaction mixture was diluted with water (150 mL) and extracted with DCM (3×150 mL). The combined organic layers were washed with brine (150 mL), dried over Na₂SO₄, filtered and concentrated under reduced pressure. The crude residue was purified by silica gel flash chromatography (120 g, gradient: Cyclohexane/EtOAc from 100:0 to 85:15) to afford B1-2-14 as a colorless oil (1.88 g, 89%).

LC/MS (AN01_001_012): Rt=2.56 min, 100%, [M+H]⁺=336.1.

### tert-butyl (2S,6S*)-2-[(benzyloxy)methyl]-6-hydroxy-6-methyl-1,4-oxazepane-4-carboxylate B1-46-1-(S)* & tert-butyl (2S,6R*)-2-[(benzyloxy)methyl]-6-hydroxy-6-methyl-1,4-oxazepane-4-carboxylate B1-46-1-(R)*

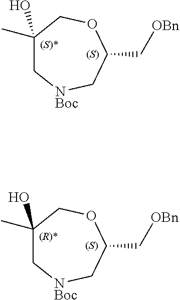

To a solution of B1-2-14 (1 eq., 1.28 g, 3.82 mmol) in anhydrous THE (35 mL) was added a 3M solution of MeMgBr in Et₂O (2.5 eq., 3.18 mL, 9.54 mmol) at 0° C. under argon atmosphere. The reaction mixture was allowed to warm to room temperature and stirred for 2 h. The reaction mixture was diluted with an aqueous saturated solution of NH₄C₁ (100 mL) and the aqueous layer was extracted with EtOAc (3×100 mL). The combined organic layers were washed with brine (100 mL), dried over Na₂SO₄, filtered and concentrated under reduced pressure. The crude residue was purified by silica gel flash chromatography (80 g, gradient: Cyclohexane/EtOAc from 100:0 to 70:30) to afford of B1-46-1-(S)* (0.650 g, 48%) and B1-46-1-(R)* (0.363 g, 27%) as colorless oils. The stereochemistry (S)* was assigned to the first eluted product by flash chromatography and then the second eluted product was assigned (R)*. B1-46-1-(S)*: LC/MS (AN01_001_012): Rt=2.47 min, 100%, [M-C₄H₈+H]⁺=296.2. B1-46-1-(R)*: LC/MS (AN01_001_012): Rt=2.41 min, 100%, [M-C₄H₈+H]⁺=296.2.

### Example 2. Synthesis of B1-46-3-(R)* and B1-46-5-(S)*

### Synthetic Scheme for the Preparation of Compound 2-A: Approach (A) w/o Protecting Group

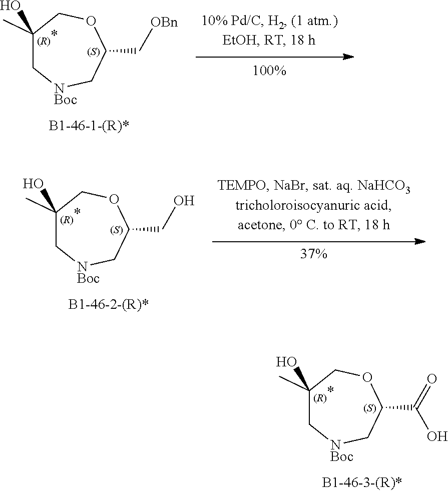

### tert-butyl (2S,6R*)-6-hydroxy-2-(hydroxymethyl)-6-methyl-1,4-oxazepane-4-carboxylate B1-46-2-(R)*

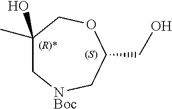

Starting from B1-46-1-(R)* (1 eq., 0.160 g, 0.455 mmol) and using general procedure A, B1-46-2-(R)* was obtained as a colorless oil (0.119 g, 100%).

LC/MS (AN01_001_012): Rt=1.83 min, non-UV active, [M+Na]⁺=284.1.

### (2S,6R*)-4-[(tert-butoxy)carbonyl]-6-hydroxy-6-methyl-1,4-oxazepane-2-carboxylic acid B1-46-3-(R)*

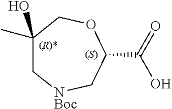

To a solution of B1-46-2-(R)* (1 eq., 0.110 g, 0.421 mmol) and sodium bromide (0.3 eq., 13.2 mg, 0.126 mmol) in acetone (7 mL) was added a saturated aqueous solution of NaHCO₃ (2 mL) at room temperature. To the resulting mixture were added trichlorocyanuric acid (2.2 eq., 0.215 mg, 0.926 mmol) and 2,2,6,6-tetramethylpiperidine-1-oxyl (0.03 eq., 1.97 mg, 0.0126 mmol) at 0° C. The reaction mixture was allowed to warm to room temperature and stirred for 18 h. Isopropanol (10 mL) was added at room temperature and the reaction mixture was stirred for 30 min. The reaction mixture was diluted with EtOAc (50 mL) and a saturated aqueous solution of NaHCO₃ (50 mL) was added. The two layers were separated, and the aqueous layer was washed with EtOAc (50 mL). The aqueous layer was then acidified with an aqueous solution of 3M HCl until pH~1 and extracted with DCM (2×50 mL). The aqueous layer was further extracted with a mixture of CHCl₃/Isopropanol (8:2, 2×50 mL). The combined organic layers were dried over Na₂SO₄, filtered and concentrated under reduced pressure to afford B1-46-3-(R)* as a yellowish oil (42.6 mg, 37%).

LC/MS (AN01_001_012): Rt=1.83 min, non-UV active, [M+Na]*=298.1.

Synthetic Scheme for the Preparation of Compound 2-B: Approach (B) with Protecting Group

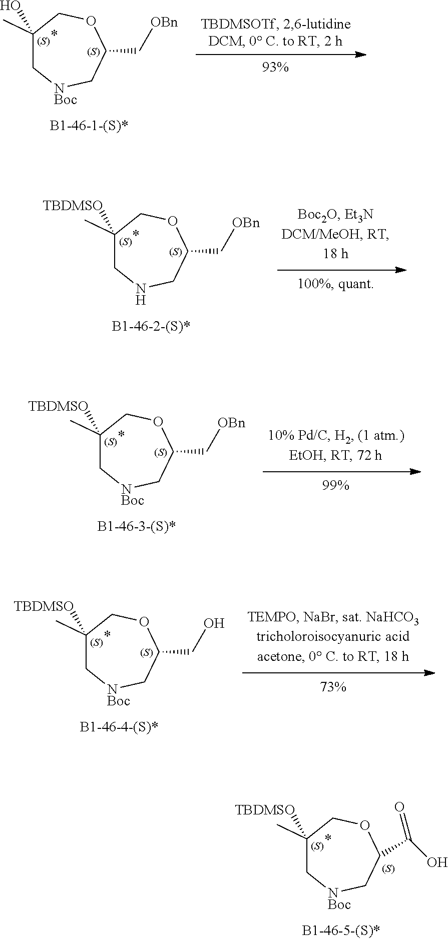

### (2S,6S*)-2-[(benzyloxy)methyl]-6-[(tert-butyldimethylsilyl)oxy]-6-methyl-1,4-oxazepane B1-46-2-(S)*

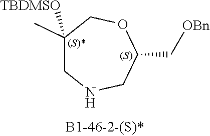

To a solution of B1-46-1-(S)* (1 eq., 0.320 g, 0.911 mmol) and 2,6-lutidine (2.5 eq., 0.265 mL, 2.28 mmol) in anhydrous DCM (3 mL) were added TBDMSOTf (1.5 eq., 0.310 mL, 1.37 mmol) and DMAP (0.05 eq., 5.56 mg, 0.0455 mmol) under argon atmosphere. The reaction mixture was stirred at room temperature for 6 h. The resulting mixture was diluted with DCM (50 mL) and water (50 mL). The two layers were separated and the aqueous layer was extracted with DCM (2×50 mL). The combined organic layers were dried over Na₂SO₄, filtered and concentrated under reduced pressure. The crude residue was purified by silica gel flash chromatography (40 g, gradient: cyclohexane/EtOAc from 100/0 to 50:50) to afford B1-46-2-(S)* as a pale-yellow oil (0.308 g, 93%).

LC/MS (AN01_001_012): Rt=2.30 min, 100%, [M+H]⁺=366.3.

### tert-butyl (2S,6S*)-2-[(benzyloxy)methyl]-6-[(tert-butyldimethylsilyl)oxy]-6-methyl-1,4-oxazepane-4-carboxylate B1-46-3-(S)*

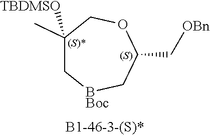

To a solution of B1-46-2-(S)* (1 eq., 0.305 g, 0.834 mmol) and Et₃N (1.01 eq., 0.117 mL, 0.843 mmol) in anhydrous MeOH (1.3 mL) was added a solution of Boc₂O (1.2 eq., 0.218 g, 1.00 mmol) in anhydrous DCM (0.25 mL) under argon atmosphere. The reaction mixture was stirred at room temperature for 18 h and then concentrated under reduced pressure to afford B1-46-3-(S)* as a colorless oil (0.389 g, quant.). The crude mixture was considered quantitative and used as such.

LC/MS (AN01_001_012): Rt=3.38 min, 100%, [M+Na]⁺=488.3.

### tert-butyl (2S,6S*)-6-[(tert-butyldimethylsilyl)oxy]-2-(hydroxymethyl)-6-methyl-1,4-oxazepane-4-carboxylate B1-46-4-(S)*

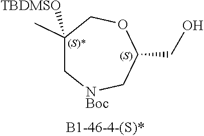

Starting from B1-46-3-(S)* (1 eq., 0.385 g, 0.827 mmol), using general procedure A, B1-46-4-(S)* was obtained as a colorless oil (0.306 g, 99%).

LC/MS (AN01_001_012): Rt=2.95 min, not UV active, [M-C₄H₈+H]⁺=320.2.

### (2S,6S*)-4-[(tert-butoxy)carbonyl]-6-[(tert-butyldimethylsilyl)oxy]-6-methyl-1,4-oxazepane-2-carboxylic acid B1-46-5-(S)*

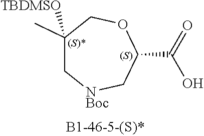

Starting from B1-46-4-(S)* (1 eq., 0.290 g, 0.772 mmol), using general procedure B, B1-46-5-(S)* was obtained as a yellowish oil (0.219 g, 73%).

LC/MS (AN01_001_012): Rt=2.84 min, not UV active, [M+Na]⁺=412.2.

### Example 3: Synthesis of B1-47-3-(S)* and B1-47-3-(R)*

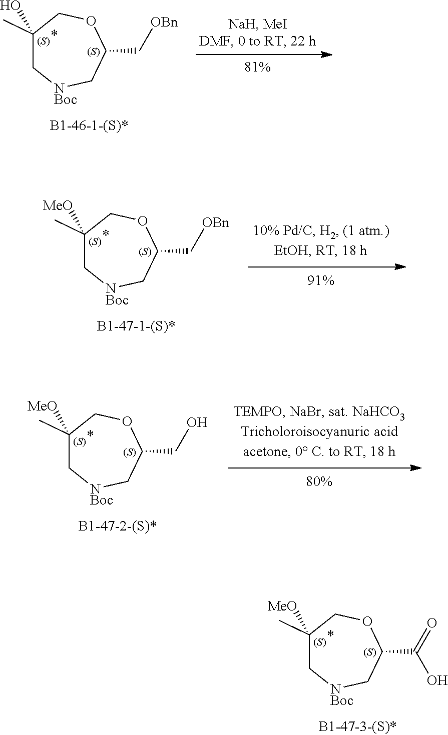

### tert-butyl (2S,6S*)-2-[(benzyloxy)methyl]-6-methoxy-6-methyl-1,4-oxazepane-4-carboxylate B1-47-1-(S)*

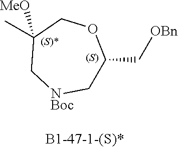

Starting from B1-46-1-(S)* (1 eq., 0.320 g, 0.910 mmol), using general procedure E, B1-47-1-(S)* was obtained as a colorless oil (0.268 g, 81%) after purification by silica gel flash chromatography (25 g, gradient: cyclohexane/EtOAc from 100:0 to 80:20).

LC/MS (AN01_001_012): Rt=2.65 min, 100%, [M+Na]⁺=388.2.

### tert-butyl (2S,6S*)-2-(hydroxymethyl)-6-methoxy-6-methyl-1,4-oxazepane-4-carboxylate B1-47-2-(S)*

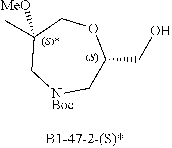

Starting from B1-47-1-(S)* (1 eq., 0.265 g, 0.725 mmol), using general procedure A, B1-47-2-(S)* was obtained as a colorless oil (0.181 g, 91%).

LC/MS (AN01_001_012): Rt=2.00 min, non-UV active, [M+Na]⁺=298.2.

### (2S,6S*)-4-[(tert-butoxy)carbonyl]-6-methoxy-6-methyl-1,4-oxazepane-2-carboxylic B1-47-3-(S)*

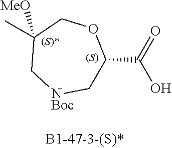

Starting from B1-47-2-(S)* (1 eq., 0.175 g, 0.636 mmol), using general procedure B, B1-47-3-(S)* was obtained as a white solid (0.147 g, 80%).

LC/MS (AN01_001_012): Rt=1.98 min, non-UV active, [M+Na]⁺=312.1.

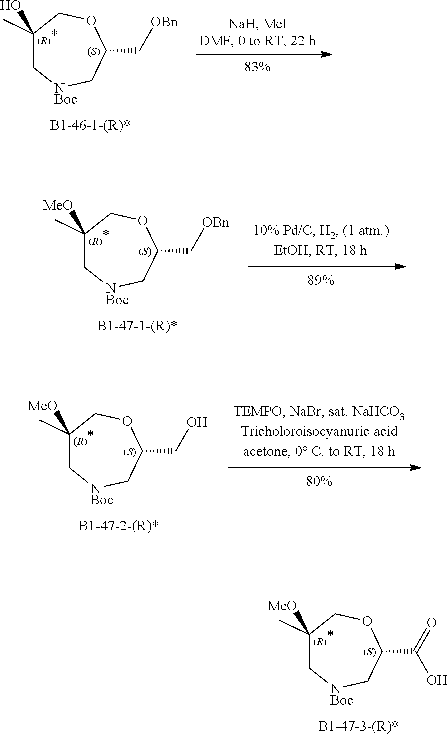

### tert-butyl (2S,6R*)-2-[(benzyloxy)methyl]-6-methoxy-6-methyl-1,4-oxazepane-4-carboxylate B1-47-1-(R)*

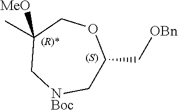

Starting from B1-46-1-(R)* (1 eq., 0.360 g, 1.02 mmol), using general procedure E, B1-47-1-(R)* was obtained as a colorless oil (0.311 g, 83%) after purification by silica gel flash chromatography (25 g, gradient: cyclohexane/EtOAc from 100:0 to 75:25).

LC/MS (AN01_001_012): Rt=2.65 min, 100%, [M+Na]*=388.2.

### tert-butyl (2S,6R*)-2-(hydroxymethyl)-6-methoxy-6-methyl-1,4-oxazepane-4-carboxylate B1-47-2-(R)*

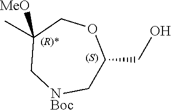

Starting from B1-47-1-(R)* (1 eq., 0.310 g, 0.848 mmol), using general procedure A, B1-47-2-(R)* was obtained as a colorless oil (0.208 g, 89%).

LC/MS (AN01_001_012): Rt=2.00 min, non-UV active, [M+Na]⁺=298.1.

### (2S,6R*)-4-[(tert-butoxy)carbonyl]-6-methoxy-6-methyl-1,4-oxazepane-2-carboxylic B1-47-3-(R)*

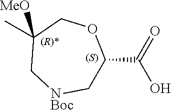

Starting from B1-47-2-(R)* (1 eq., 0.205 g, 0.744 mmol), using general procedure B, B1-47-3-(R)* was obtained as a white solid (0.124 g, 58%).

LC/MS (ANO 1_001_012): Rt=2.00 min, non-UV active, [M+Na]⁺=312.1.

### Example 4: Synthesis of H1-2-7

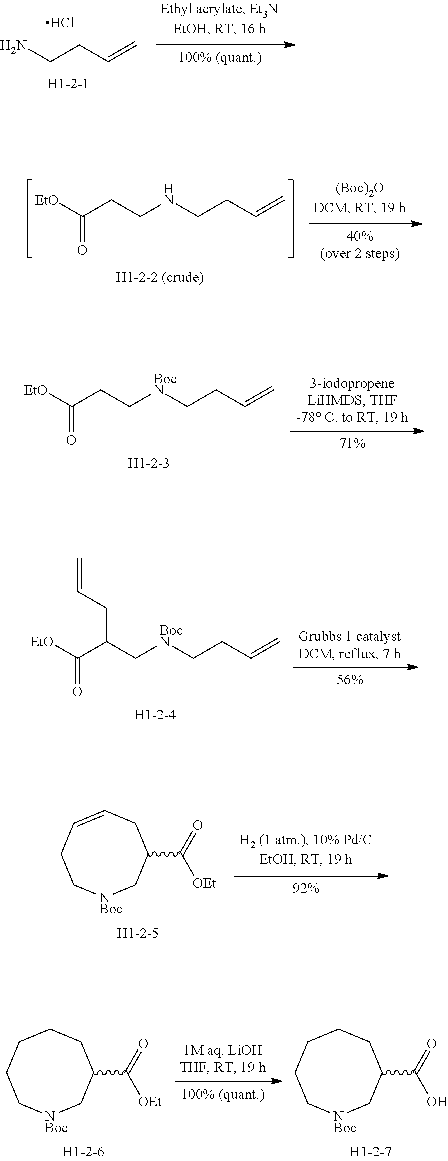

### Ethyl 3-[(but-3-en-1-yl)[(tert-butoxy)carbonyl]amino]propanoate H1-2-3

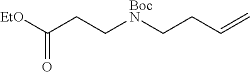 [SMILES: C=CCCN(CCC(=O)OCC)C(=O)OC(C)(C)C]

An argon-purged solution of but-3-en-1-amine hydrochloride H1-2-1 (1 eq., 1.99 g, 18.5 mmol) and triethylamine (1.05 eq., 2.70 mL, 19.4 mmol) in EtOH (28 mL) was stirred at room temperature for 30 min. Ethyl acrylate (1 eq., 2.01 mL, 18.5 mmol) was then added and the resulting mixture was stirred at room temperature for 17 h. The reaction mixture was concentrated under reduced pressure to afford a crude mixture containing H1-2-2 (4.88 g, estimated purity: 50%) as a colorless oil.

To an argon-purged solution of the crude mixture containing H1-2-2 (4.88 g, estimated purity: 50%, 14.2 mmol, 1 eq.) in DCM (30 mL) were added diisopropylamine (1.2 eq., 2.42 mL, 17.1 mmol), Boc₂O (1.2 eq., 3.73 g, 17.1 mmol) and DMAP (0.1 eq., 0.170 g, 1.42 mmol) at room temperature. The resulting mixture was stirred at room temperature for 19 h. The reaction mixture was diluted with water (50 mL) and extracted with DCM (2×50 mL). The combined organic layers were washed with brine (25 mL), dried over Na₂SO₄, filtered and concentrated under reduced pressure. The resulting yellow oil was purified twice by silica gel flash chromatography (1^{st} flash chromatography: 120 g, gradient: cyclohexane/EtOAc from 100:0 to 80:20; 2^{nd} flash chromatography: 40 g, gradient: cyclohexane/DCM from 80:20 to 0:100, then DCM/EtOAC from 100:0 to 80:20) to afford H1-2-3 as a colorless oil (1.99 g, 40% over 2 steps).

LC/MS (AN01_001_012): Rt=2.65 min, 100%, [M-C₄H₈+H]⁺=216.2.

### Ethyl 2-{[(but-3-en-1-yl)[(tert-butoxy)carbonyl]amino]methyl}pent-4-enoate H₁₋₂-4

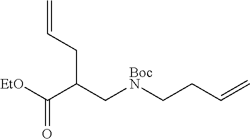 [SMILES: C=CCCN(CC(CC=C)C(=O)OCC)C(=O)OC(C)(C)C]

To an argon-purged solution of H1-2-3 (1 eq., 1.99 g, 7.33 mmol) in THE (20 mL) was added a 1M LiHMDS solution in THE (1.1 eq., 8.07 mL, 8.07 mmol) dropwise at −78° C. The resulting mixture was stirred at −78° C. for 1 h before the dropwise addition of allyl iodide (1.1 eq., 0.740 mL, 8.07 mmol). The resulting mixture was allowed to warm to room temperature and stirred for 15 h. The reaction mixture was cooled to −78° C. and a 1M LiHMDS solution in THF (0.2 eq., 1.47 mL, 1.47 mmol) was added dropwise at −78° C. The reaction was stirred at this temperature for 30 min before the dropwise addition of allyl iodide (0.2 eq., 0.135 mL, 1.47 mmol). The resulting mixture was allowed to warm to room temperature and stirred for 3 h. The reaction mixture was diluted with water (100 mL) and extracted with EtOAc (3×100 mL). The combined organic layers were washed with brine (100 mL), dried over Na₂SO₄, filtered and concentrated under reduced pressure. The resulting orange oil was purified by silica gel flash chromatography (80 g, gradient: cyclohexane/EtOAc from 100:0 to 80:20) to afford H1-2-4 as a yellow oil (1.62 g, 71%).

LC/MS (AN01_001_012): Rt=2.85 min, 100%, [M-C₅H₈O₂+H]⁺=212.2.

### 1-tert-butyl 3-ethyl 1,2,3,4,7,8-hexahydroazocine-1,3-dicarboxylate H₁₋₂-5

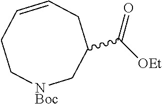 [SMILES: CCOC(=O)C1C/C=C\CCN(C(=O)OC(C)(C)C)C1]

To an argon-purged solution of H1-2-4 (1 eq., 1.20 g, 3.85 mmol) in DCM (200 mL) was added benzylidene-bis(tricyclohexylphosphine)dichlororuthenium (0.1 eq., 0.318 g, 0.385 mmol) at room temperature. The resulting mixture was stirred and refluxed for 7 h. The reaction mixture was cooled to room temperature and concentrated under reduced pressure. The resulting black oil was purified by silica gel flash chromatography (80 g, gradient: cyclohexane/EtOAc from 100:0 to 80:20) to afford H1-2-5 as a black oil (0.613 g, 56%).

LC/MS (AN01_001_012): Rt=2.67 min, 81%, [M-C₄H₈+H]⁺=228.1.

### 1-tert-butyl 3-ethyl azocane-1,3-dicarboxylate H1-2-6

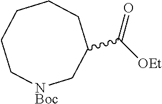 [SMILES: CCOC(=O)C1CCCCCN(C(=O)OC(C)(C)C)C1]

To an argon-purged solution of H1-2-5 (1 eq., 0.350 g, 1.24 mmol) in EtOH (6 mL) was added 10% Pd/C (0.2 eq., 0.263 g, 0.247 mmol) at room temperature. The resulting mixture was purged with argon (×3) and then with H₂ (3×). The reaction mixture was stirred under an atmospheric pressure of H₂ at room temperature for 19 h. The reaction mixture was purged with argon, filtered on a pad of celite and rinsed with EtOH (2×15 mL). The filtrate was concentrated under reduced pressure to afford H1-2-6 as a yellow oil (0.323 g, 92%).

LC/MS (AN01_001_012): Rt=2.73 min, non-UV active, [M-C₄H₈+H]⁺=230.1.

### 1-[(tert-butoxy)carbonyl]azocane-3-carboxylic acid H1-2-7

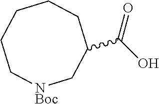 [SMILES: CC(C)(C)OC(=O)N1CCCCCC(C(=O)O)C1]

To a solution of H1-2-6 (1 eq., 0.323 g, 1.13 mmol) in THE (11 mL) was added a solution of LiOH (5 eq., 0.237 g, 5.66 mmol) in water (5.5 mL) at room temperature. The reaction mixture was stirred at room temperature for 19 h. The reaction mixture was poured dropwise into a stirred mixture of a 1M HCl aqueous solution (50 mL) and DCM (100 mL) at 0° C. The resulting mixture was stirred for 1 h (pH~1) and the layers were separated. The aqueous layer was extracted with DCM (2×50 mL) and the combined organic layers were dried over Na₂SO₄, filtered and concentrated under reduced pressure to afford H1-2-7 as a yellow oil (0.291 g, 100%).

LC/MS (ANO 1_001_012): Rt=2.29 min, non-UV active, [M+Na]⁺=280.2.

### Example 5. General scheme for the synthesis of (S)—N—((S)-1-cyano-2-(4-(3-methyl-2-oxo-2,3-dihydrobenzo[d]oxazol-5-yl)bicyclo[2.2.2]octan-1-yl)ethyl)-1,4-oxazepane-2-carboxamide

(S)—N—((S)-1-cyano-2-(4-(3-methyl-2-oxo-2,3-dihydrobenzo[d]oxazol-5-yl)bicyclo[2.2.2]octan-1-yl)ethyl)-1,4-oxazepane-2-carboxamide can be prepared according to the scheme below.

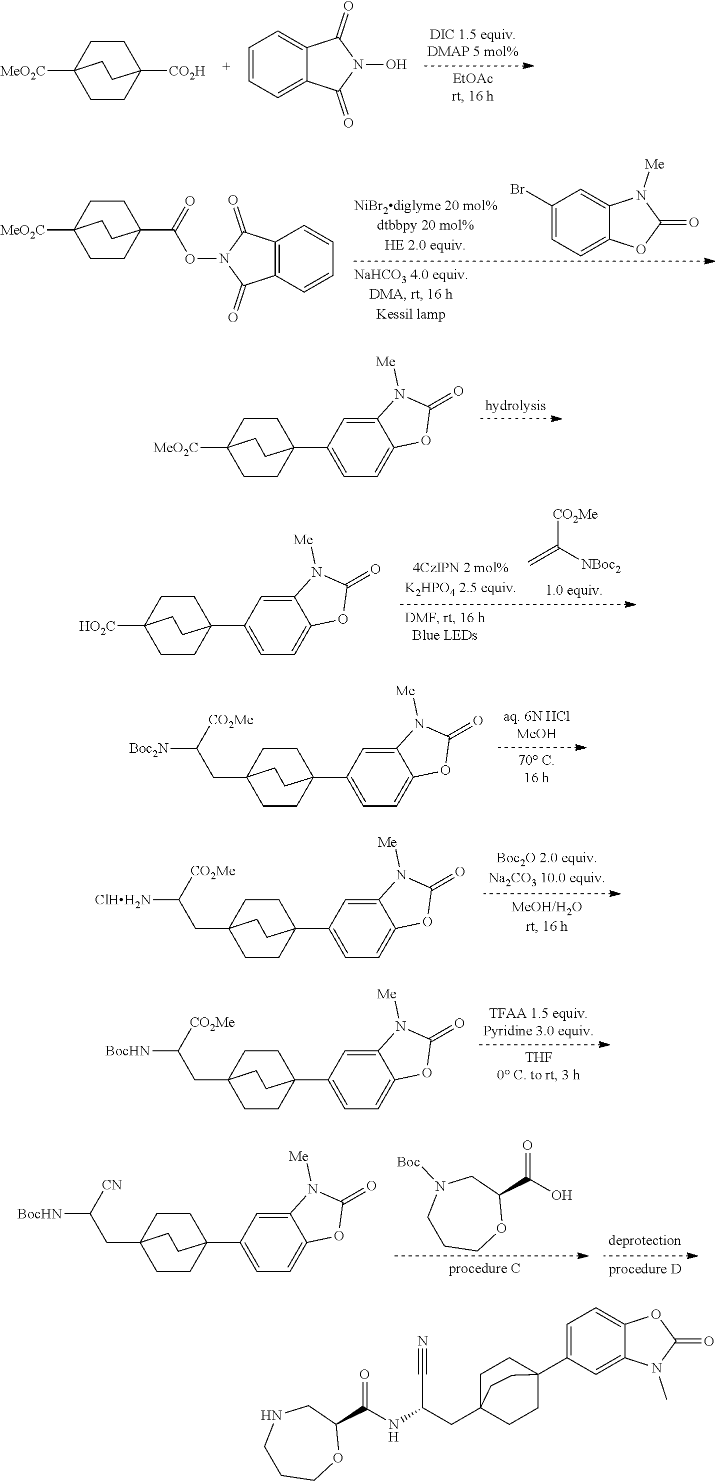

### Example 6. General scheme for the synthesis of (2S)—N-(1-cyano-2-(6-(3-methyl-2-oxo-2,3-dihydrobenzo[d]oxazol-5-yl)benzo[b]thiophen-2-yl)ethyl)-1,4-oxazepane-2-carboxamide

(2S)—N-(1-cyano-2-(6-(3-methyl-2-oxo-2,3-dihydrobenzo[d]oxazol-5-yl)benzo[b]thiophen-2-yl)ethyl)-1,4-oxazepane-2-carboxamide can be prepared according to the scheme below.

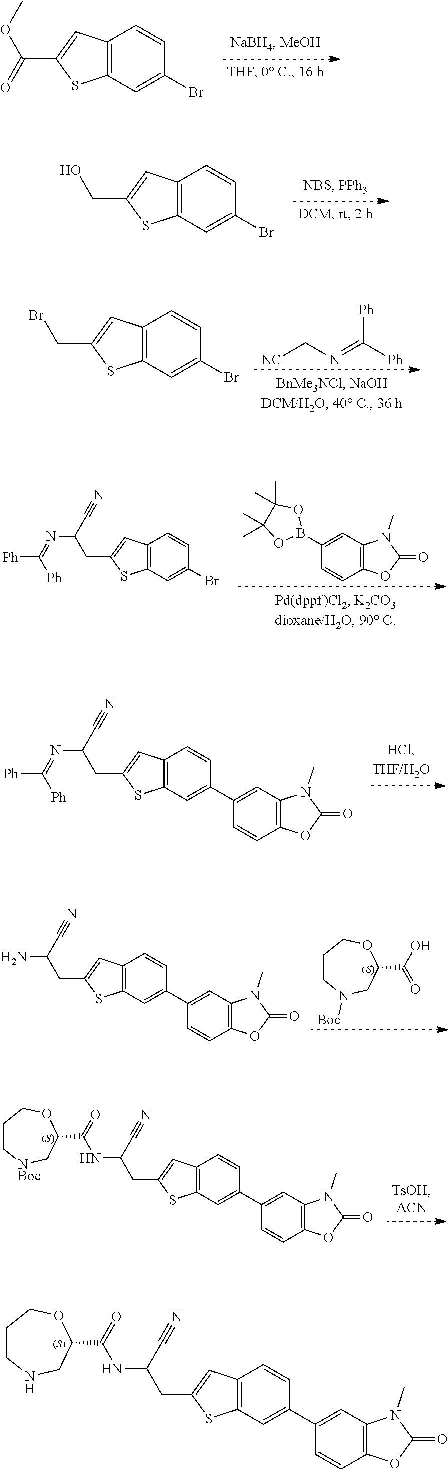

### Example 7. General scheme for the synthesis of (2S)—N-(1-cyano-2-(2-(3-methyl-2-oxo-2,3-dihydrobenzo[d]oxazol-5-yl)benzo[b]thiophen-6-yl)ethyl)-1,4-oxazepane-2-carboxamide

(2S)—N-(1-cyano-2-(2-(3-methyl-2-oxo-2,3-dihydrobenzo[d]oxazol-5-yl)benzo[b]thiophen-6-yl)ethyl)-1,4-oxazepane-2-carboxamide can be prepared according to the scheme below.

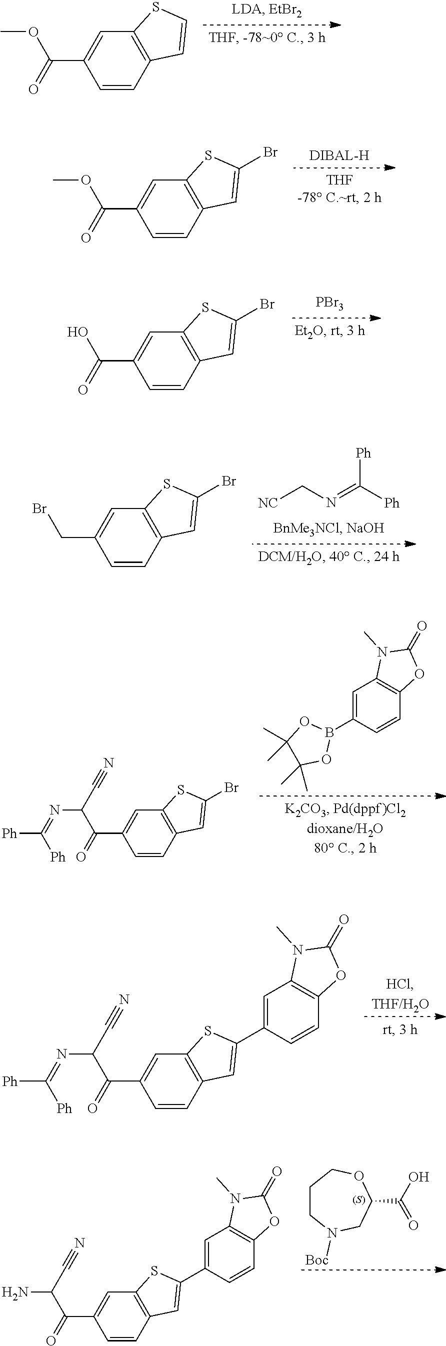

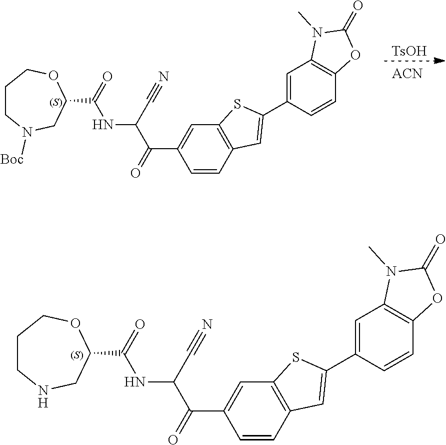

### Example 8: IC50 Assays

### Mouse DPP1 Enzyme IC50 Assay

Test articles are applied to active mouse DPP1 enzyme (R&D Systems; Minneapolis, MN) in Assay Buffer (50 mM MES pH 5.5, 50 mM NaCl, 5 mM DTT) in a total reaction volume of 125 μL. 25 μL of compound in Assay Buffer plus 5% DMSO is first added to 50 μL of active mouse DPP1 enzyme at a concentration of 62.5 pg/μL and allowed to pre-incubate for 10 minutes at 37° C. after which 50 μL of 1000 μM H-Gly-Arg-AMC substrate (Bachem; St. Torrance, CA) is added, giving final substrate concentration of 400 μM and a final DMSO concentration of 1%. Substrate cleavage is measured for 90 minutes at 37° C., with fluorescence at Excitation/Emission 350/450 nm measured every 5 minutes. DPP1 concentration is interpolated based on its activity relative to a standard curve of recombinant active mouse DPP1 enzyme. IC50 values for each compound are calculated via the XLFit (IDBS Version 5.3.1.3) Add-On to Microsoft Excel using the four parameter fit equation y=(A+((B-A)/(1+((C/x)\hat{ }D)))), which appears as equation number 205 (4 Parameter Logistic Model or Sigmoidal Dose-Response Model) in XLFit. Default constraints are used for each Parameter. IC50 is defined as the compound concentration at which 50% of enzyme activity is inhibited when compared to the no-compound control.

### Human DPP1 Enzyme IC50 Assay

Recombinant human DPP1 enzyme (R&D Systems; Minneapolis, MN) is first proteolytically processed into its mature form using recombinant human cathepsin L (R&D Systems) in a buffer consisting of 20 mM citric acid pH 4.5, 150 mM NaCl, 1 mM EDTA and 10 mM DTT. Test articles are applied to activated human DPP1 enzyme in Assay Buffer (25 mM MES pH 6.0, 50 mM NaCl, 5 mM DTT) in a total reaction volume of 125 μL. 25 μL of compound in Assay Buffer plus 5% DMSO is first added to 50 μL of activated human DPP1 enzyme at a concentration of 1 ng/μL and allowed to pre-incubate for 10 minutes at 37° C. after which 50 μL of 1000 μM H-Gly-Arg-AMC substrate (Bachem; St. Torrance, CA) is added, giving final substrate concentration of 400 μM and a final DMSO concentration of 1%. Substrate cleavage is measured for 90 minutes at 37° C., with fluorescence at Excitation/Emission 350/450 nm measured every 5 minutes. DPP1 concentration is interpolated based on its activity relative to a standard curve of activated human recombinant DPP1 enzyme. IC50 values for each compound are calculated via the XLFit (IDBS Version 5.3.1.3) Add-On to Microsoft Excel using the four parameter fit equation y={A+[(B−A)]/[1+((C/x)\hat{ }D)]}, which appears as equation number 205 (4 Parameter Logistic Model or Sigmoidal Dose-Response Model) in XLFit. Default constraints are used for each Parameter. IC50 is defined as the compound concentration at which 5000 of enzyme activity is inhibited when compared to the no-compound control. The IC50 table is included in the table below.

TABLE 2
DPP1 IC₅₀ values ^{[1]}

| | DPP1 | DPP1 | Cell |
|---|---|---|---|
| Compound | IC50, nM; | IC50, nM; | Assay, |
| No. | Human^{[2]} | Mouse^{[2]} | nM[^{2]} |
| 112b | * | NA | NA |
| 131b | * | NA | * |
| 113b | * (n = 3) | * (n = 3) | * (n = 3) |
| 114b | * (n = 3) | * (n = 3) | * (n = 3) |
| 134a | * | NA | * |
| 130b | * (n = 2) | NA | * (n = 2) |
| 115b | * (n = 3) | * (n = 3) | * (n = 3) |
| 113b | * (n = 3) | NA | * (n = 2) |
| 133b | * | NA | * |
| 118b | * (n = 3) | * (n = 3) | * (n = 3) |
| 128b | * (n = 3) | NA | * (n = 3) |
| 101d | * | NA | NA |
| 138b | * (n = 3) | NA | * |
| 138c | * | NA | * |
| 139b | * (n = 3) | NA | * |
| 139c | * | NA | * |
| 142b | ** (n = 3) | NA | * (n = 2) |
| | DPP1 | DPP1 | Cell |
| | IC50, nM; | IC50, nM; | Assay, |
| symbol | Human | Mouse | nM |
| *** | ≤5 | ≤5 | ≤1 |
| ** | 5-15 | 5-20 | 1-1.5 |
| * | ≥15 | ≥20 | ≥1.5 |

^{[1]} NA means the data for this compound is not available.
^{[2]}The symbols correspond to ranges of IC50 according to the table below.

### Example 9. Synthesis of compound 112a: (2S)—N-{1-cyano-2-[2-(3-methyl-2-oxo-1,3-benzoxazol-5-yl)-1-benzothiophen-6-yl]ethyl}-1,4-oxazepane-2-carboxamide

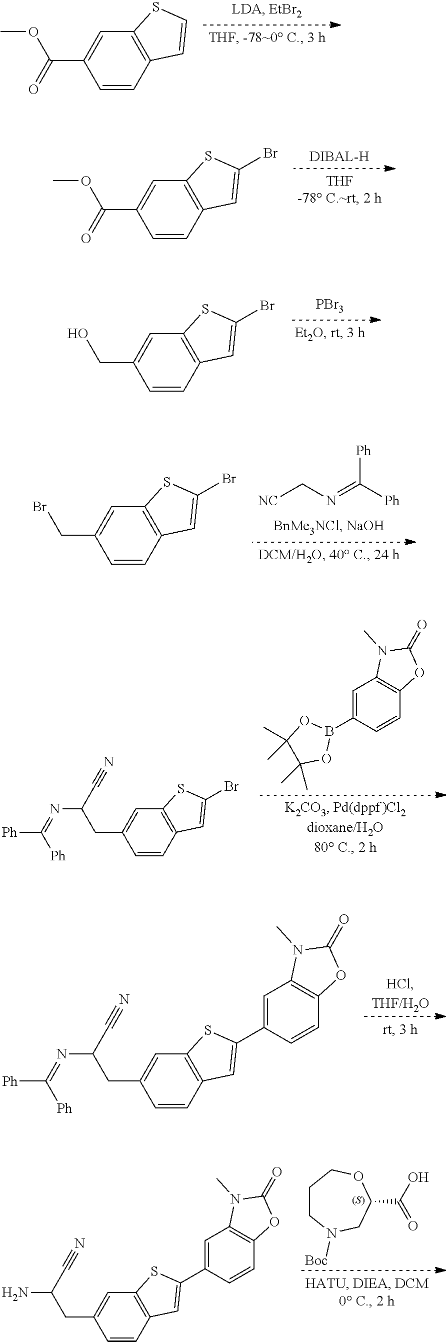

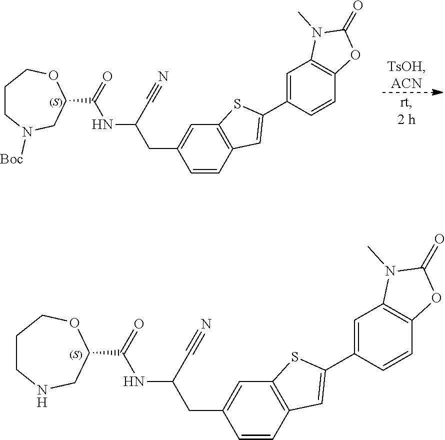

### Synthesis of methyl 2-bromo-1-benzothiophene-6-carboxylate

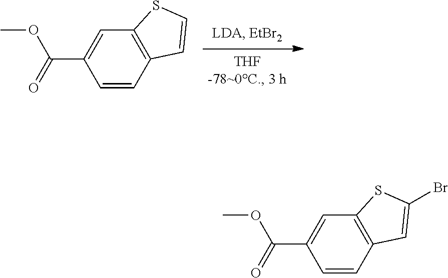

To a stirred solution of methyl 1-benzothiophene-6-carboxylate (2 g, 10.404 mmol, 1 equiv) and THE (30 mL) was added LDA (in 2M THF) (6.24 mL, 12.49 mmol, 1.2 equiv) dropwise at −78° C. under nitrogen atmosphere. The resulting mixture was stirred for 1 h at −78° C. under nitrogen atmosphere. To the above mixture was added dibromoethane (2.15 g, 11.44 mmol, 1.1 equiv) dropwise at −78° C. under nitrogen atmosphere. The resulting mixture was stirred for additional 1 h at room temperature under nitrogen atmosphere. The reaction was quenched by the addition of Water (50 mL) at room temperature. The resulting mixture was extracted with EtOAc (3×50 mL). The combined organic layers were dried over anhydrous Na₂SO₄. After filtration, the filtrate was concentrated under reduced pressure. The residue was purified by silica gel column chromatography, eluted with PE/EA (12:1) to afford methyl 2-bromo-1-benzothiophene-6-carboxylate (1.7 g, 60.26%) as a light-yellow oil. (NO MS signal in LCMS)

¹H NMR (300 MHz, DMSO-d₆) δ 8.63 (dt, J=1.6, 0.8 Hz, 1H), 7.95 (dd, J=8.4, 1.4 Hz, 1H), 7.91 (dd, J=8.3, 0.8 Hz, 1H), 7.77 (d, J=0.7 Hz, 1H), 3.89 (s, 3H).

### Synthesis of (2-bromo-1-benzothiophen-6-yl)methanol

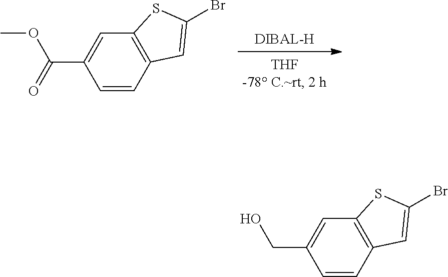

A solution of methyl 2-bromo-1-benzothiophene-6-carboxylate (0.9 g, 3.32 mmol, 1 equiv) and DIBAL-H (9.96 mL, 9.96 mmol, 3 equiv) in tetrahydrofuran (40 mL, 14.38 mmol) was stirred for 2 h at −78° C.-rt under nitrogen atmosphere. The reaction was quenched with HCl (1 M) at 0° C. The resulting mixture was extracted with EtOAc (3×100 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EA (3:1) to afford (2-bromo-1-benzothiophen-6-yl)methanol (0.65 g, 80.54%) as a white solid. LCMS (ES, m/z): [M−18+H]⁺: 225

¹H NMR (300 MHz, DMSO-d₆) δ 7.86 (s, 1H), 7.75 (d, J=8.2 Hz, 1H), 7.61 (s, 1H), 7.34 (dd, J=8.2, 1.5 Hz, 1H), 5.30 (t, J=5.7 Hz, 1H), 4.59 (d, J=5.7 Hz, 2H).

### Synthesis of 2-bromo-6-(bromomethyl)-1-benzothiophene

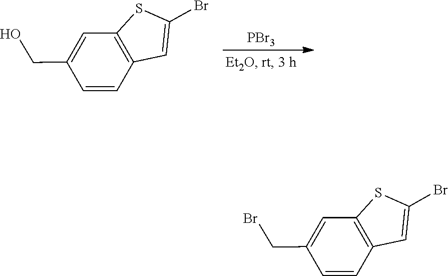

A solution of (2-bromo-1-benzothiophen-6-yl)methanol (0.65 g, 2.674 mmol, 1 equiv) in Et₂O (10 mL) was added PBr₃ (0.36 g, 1.337 mmol, 0.5 equiv) at 0° C. The resulting mixture was stirred for additional 2 h at room temperature. The reaction was quenched by the addition of Water at 0° C. The resulting mixture was extracted with EtOAc (3×50 mL). The combined organic layers were washed with brine (1×50 mL), dried over anhydrous Na₂SO₄. After filtration, the filtrate was concentrated under reduced pressure. This resulted in 2-bromo-6-(bromomethyl)-1-benzothiophene (700 mg, 85.56%) as a white solid. (NO MS signal in LCMS)

¹H NMR (300 MHz, DMSO-d₆) δ 8.05 (d, J=1.6 Hz, 1H), 7.80 (d, J=8.2 Hz, 1H), 7.66 (s, 1H), 7.46 (dd, J=8.2, 1.7 Hz, 1H), 4.83 (s, 2H).

### Synthesis of 3-(2-bromo-1-benzothiophen-6-yl)-2-[(diphenylmethylidene)amino]propanenitrile

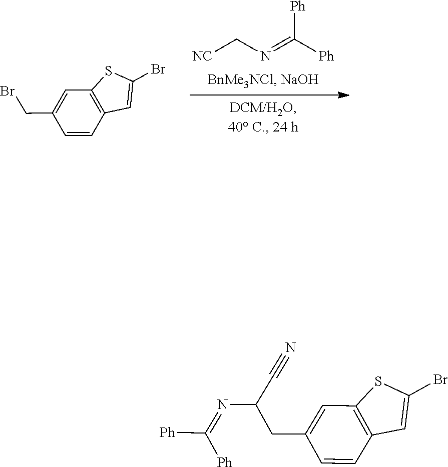 [SMILES: N#CCN=C(c1ccccc1)c1ccccc1 ; BrCc1ccc2cc(Br)sc2c1 ; N#CC(Cc1ccc2cc(Br)sc2c1)N=C(c1ccccc1)c1ccccc1]

A solution of 2-bromo-6-(bromomethyl)-1-benzothiophene (600 mg, 1.96 mmol, 1.0 equiv) in DCM (5 mL) was added 2-[(diphenylmethylidene)amino]acetonitrile (432 mg, 1.96 mmol, 1.0 equiv), benzyltrimethylazanium chloride (36.41 mg, 0.196 mmol, 0.1 equiv), NaOH (156 mg, 3.92 mmol, 2.0 equiv) in H₂O (1 mL) was stirred for 36 h at 40° C. The resulting mixture was extracted with CH₂Cl₂ (3×100 mL). The combined organic layers were washed with brine (3×30 mL), dried over anhydrous Na₂SO₄. After filtration, the filtrate was concentrated under reduced pressure. The residue was purified by silica gel column chromatography, eluted with PE/THF (5:1) to afford 3-(2-bromo-1-benzothiophen-6-yl)-2-[(diphenylmethylidene)amino]propanenitrile (800 mg crude) as a yellow solid. LCMS (ES, m/z): [M+H]⁺: 445.

### Synthesis of 2-[(diphenylmethylidene)amino]-3-[2-(3-methyl-2-oxo-1,3-benzoxazol-6-yl)-1-benzothiophen-5-yl]propanenitrile

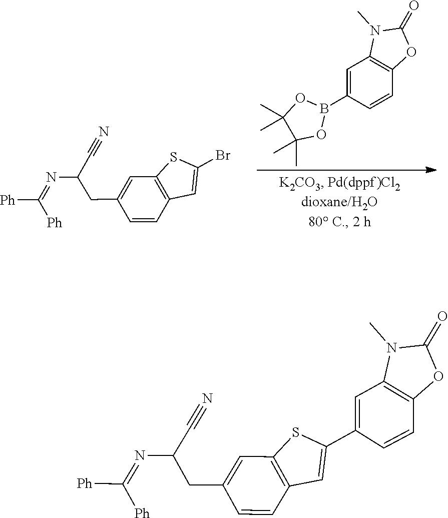 [SMILES: N#CC(Cc1ccc2cc(Br)sc2c1)N=C(c1ccccc1)c1ccccc1 ; Cn1c(=O)oc2ccc(B3OC(C)(C)C(C)(C)O3)cc21 ; Cn1c(=O)oc2ccc(-c3cc4ccc(CC(C#N)N=C(c5ccccc5)c5ccccc5)cc4s3)cc21]

A solution of 3-(2-bromo-1-benzothiophen-6-yl)-2-[(diphenylmethylidene)amino]propanenitrile (800 mg, 1.80 mmol, 1.0 equiv), 3-methyl-5-(4,4,5-trimethyl-1,3,2-dioxaborolan-2-yl)-1,3-benzoxazol-2-one (468 mg, 1.80 mmol, 1.0 equiv), K₂CO₃ (496 mg, 3.59 mmol, 2.0 equiv) and Pd(dppf)Cl₂ (131 mg, 0.18 mmol, 0.1 equiv) in 1,4-dioxane (10 mL), H₂O (1 mL) was stirred for 2 h at 80° C. under nitrogen atmosphere. The residue was concentrated under reduced pressure. The residue was purified by silica gel column chromatography, eluted with PE/THF (5:1) to afford 2-[(diphenylmethylidene)amino]-3-[2-(3-methyl-2-oxo-1,3-benzoxazol-6-yl)-1-benzothiophen-5-yl]propanenitrile (600 mg, 65.04%) as a white solid. LCMS (ES, m/z): [M+H]⁺: 514.

### Synthesis of 2-amino-3-[2-(3-methyl-2-oxo-1,3-benzoxazol-5-yl)-1-benzothiophen-6-yl]propanenitrile

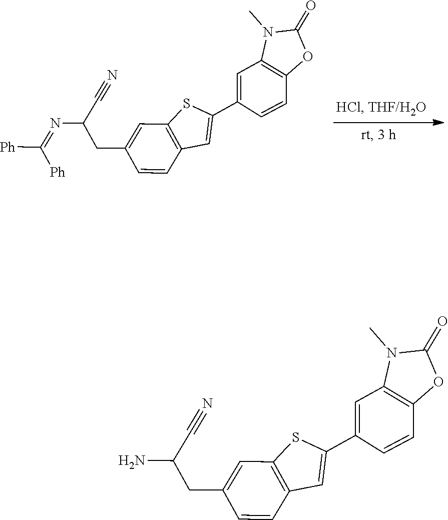 [SMILES: Cn1c(=O)oc2ccc(-c3cc4ccc(CC(C#N)N=C(c5ccccc5)c5ccccc5)cc4s3)cc21 ; Cn1c(=O)oc2ccc(-c3cc4ccc(CC(N)C#N)cc4s3)cc21]

Into a 100 mL round-bottom flask were added 2-[(diphenylmethylidene)amino]-3-[2-(3-methyl-2-oxo-1,3-benzoxazol-5-yl)-1-benzothiophen-6-yl]propanenitrile (600 mg, 1.17 mmol, 1 equiv) and THE (50 mL), H₂O (5 mL) at room temperature. The resulting mixture was stirred for additional 3 h at room temperature. The mixture was basified to pH 12 with NaOH. The resulting mixture was extracted with EtOAc (3×50 mL). The combined organic layers were washed with brine (3×30 mL), dried over anhydrous Na₂SO₄. After filtration, the filtrate was concentrated under reduced pressure. The residue was purified by silica gel column chromatography, eluted with PE/THF (1:2) to afford 2-amino-3-[2-(3-methyl-2-oxo-1,3-benzoxazol-5-yl)-1-benzothiophen-6-yl]propanenitrile (300 mg, 73.50%) as a white solid. LCMS (ES, m/z): [M+H]⁺: 350.

### Synthesis of tert-butyl (2S)-2-({1-cyano-2-[2-(3-methyl-2-oxo-1,3-benzoxazol-5-yl)-1-benzothiophen-6-yl]ethyl}carbamoyl)-1,4-oxazepane-4-carboxylate

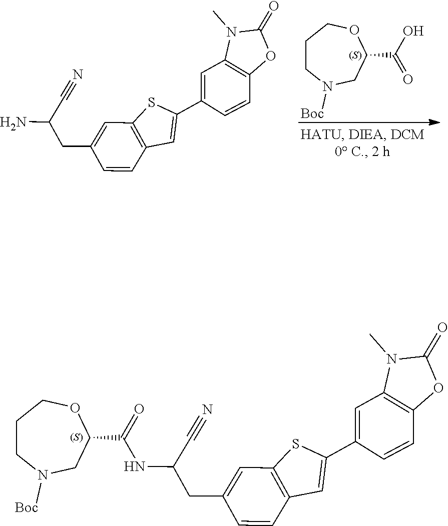

To a stirred mixture of 2-amino-3-[2-(3-methyl-2-oxo-1,3-benzoxazol-5-yl)-1-benzothiophen-6-yl]propanenitrile (110 mg, 0.32 mmol, 1.2 equiv), (2S)-4-(tert-butoxycarbonyl)-1,4-oxazepane-2-carboxylic acid (64 mg, 0.26 mmol, 1.0 equiv) and DIEA (102 mg, 0.79 mmol, 3.0 equiv) in DCM (5 mL) were added HATU (120 mg, 0.32 mmol, 1.2 equiv) at 0° C. The resulting mixture was stirred for additional 2 h at 0° C. The residue was purified by silica gel column chromatography, eluted with PE/THF (1:1) to afford tert-butyl (2S)-2-({1-cyano-2-[2-(3-methyl-2-oxo-1,3-benzoxazol-5-yl)-1-benzothiophen-6-yl]ethyl}carbamoyl)-1,4-oxazepane-4-carboxylate (120 mg, 79.32%) as a light yellow oil. LCMS (ES, m/z): [M+H]⁺: 577.

### Synthesis of (2S)—N-{1-cyano-2-[2-(3-methyl-2-oxo-1,3-benzoxazol-5-yl)-1-benzothiophen-6-yl]ethyl}-1,4-oxazepane-2-carboxamide

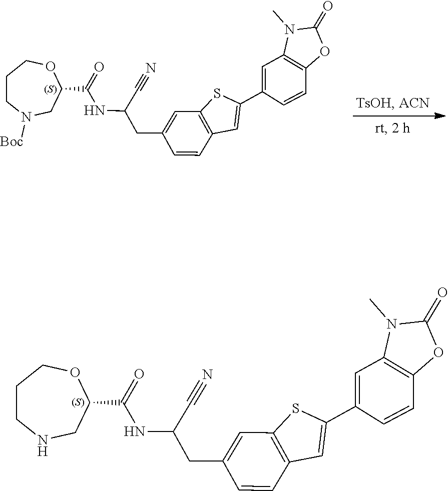

Into a 50 mL round-bottom flask were added tert-butyl (2S)-2-({1-cyano-2-[2-(3-methyl-2-oxo-1,3-benzoxazol-5-yl)-1-benzothiophen-6-yl]ethyl}carbamoyl)-1,4-oxazepane-4-carboxylate (120 mg, 0.21 mmol, 1 equiv), TsOH (108 mg, 0.62 mmol, 3.0 equiv) and ACN (3 mL) at room temperature. The resulting mixture was stirred for additional 3 h at room temperature. The residue was purified by reversed-phase flash chromatography with the following conditions: column, C₁₈ silica gel; mobile phase, MeCN in Water (0.1% NH₃·H₂O), 10% to 50% gradient in 10 min; detector, UV 254 nm. This resulted in (2S)—N-{1-cyano-2-[2-(3-methyl-2-oxo-1,3-benzoxazol-5-yl)-1-benzothiophen-6-yl]ethyl}-1,4-oxazepane-2-carboxamide (18.5 mg, 18.66%) as a white solid.

Analytical Data LCMS (ES, m/z): [M+H]⁺: 477.

¹H NMR (400 MHz, DMSO-d₆) δ 8.62 (dd, J=11.8, 8.5 Hz, 1H), 7.91-7.84 (m, 2H), 7.79 (dd, J=8.2, 2.9 Hz, 1H), 7.73 (d, J=1.9 Hz, 1H), 7.51 (dt, J=8.3, 2.0 Hz, 1H), 7.42 (d, J=8.3 Hz, 1H), 7.33 (dt, J=8.1, 1.7 Hz, 1H), 5.13-4.93 (m, 1H), 4.01-3.80 (m, 2H), 3.78-3.65 (m, 1H), 3.43 (s, 3H), 3.30-3.27 (m, 2H), 3.05 (ddd, J=52.3, 14.2, 3.7 Hz, 1H), 2.84-2.63 (m, 2H), 2.60-2.53 (m, 1H), 2.49-2.41 (m, 1H), 1.79-1.65 (m, 2H).

### Example 10. Synthesis of compound 113a: (2S)—N-(1-cyano-2-(6-(3-methyl-2-oxo-2,3-dihydrobenzo[d]oxazol-5-yl)benzo[b]thiophen-2-yl)ethyl)-1,4-oxazepane-2-carboxamide

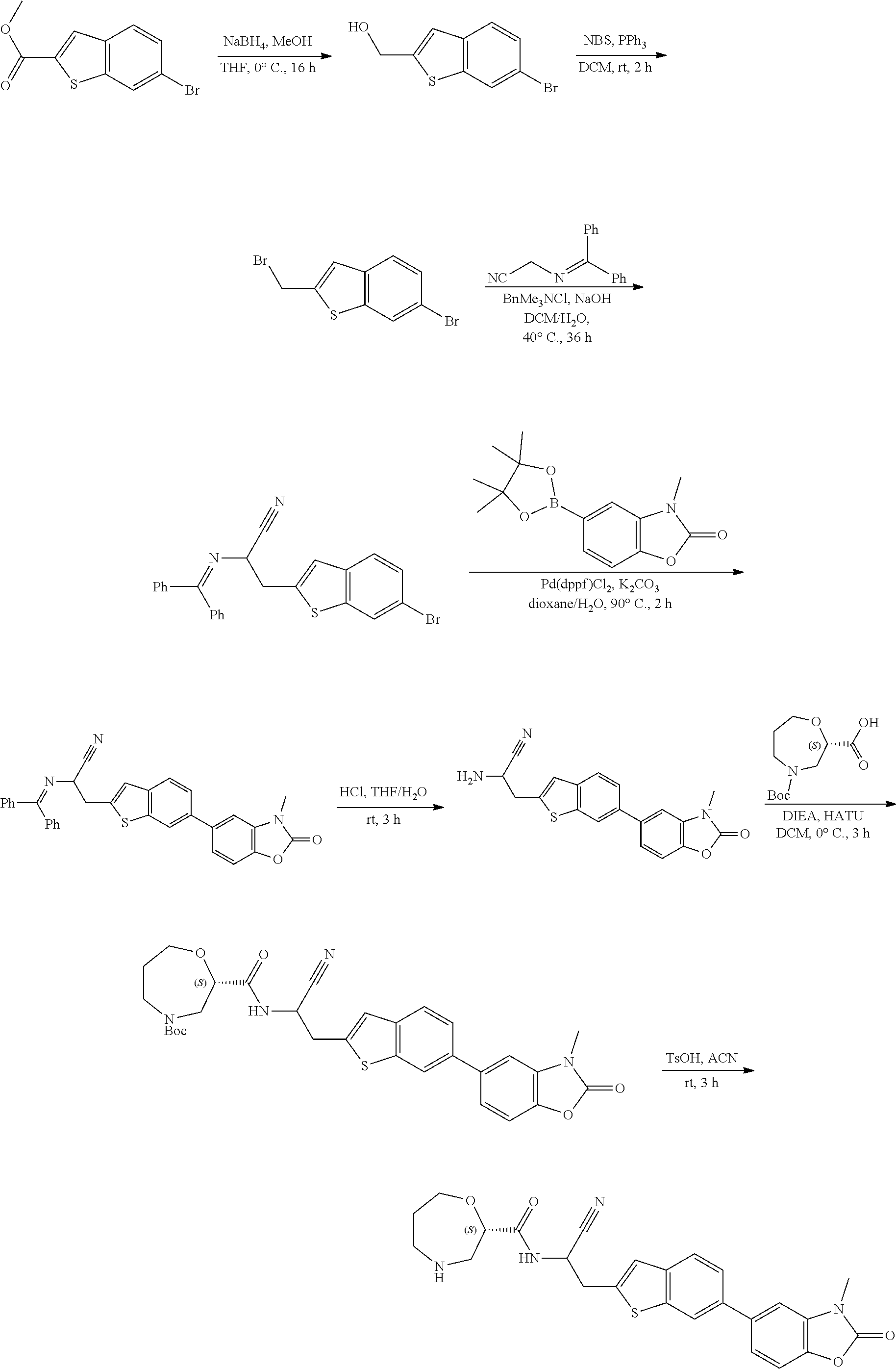

### Synthesis of (6-bromo-1-benzothiophen-2-yl)methanol

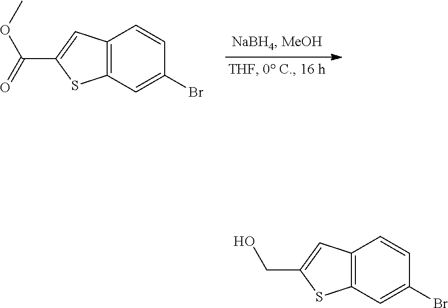

To a solution of methyl 6-bromo-1-benzothiophene-2-carboxylate (4.0 g, 14.75 mmol, 1.0 equiv) in MeOH (30.0 mL) and THF (60.0 mL), and NaBH₄ (1.1 g, 29.50 mmol, 2.0 equiv) was added at 0° C. The mixture was stirred for 16 h at 0° C. The reaction was quenched with water (100 mL), extracted with EtOAc (3×100 mL). The combined organic layer was washed with brine (3×30 mL), dried over anhydrous Na₂SO₄. After filtration, the filtrate was concentrated under reduced pressure. The residue was purified by silica gel column chromatography, eluted with PE/THF (5:1) to afford (6-bromo-1-benzothiophen-2-yl)methanol (3.3 g, 92%) as a yellow green solid. LCMS (ES, m/z): [M+H]⁺: 243.

### Synthesis of 6-bromo-2-(bromomethyl)-1-benzothiophene

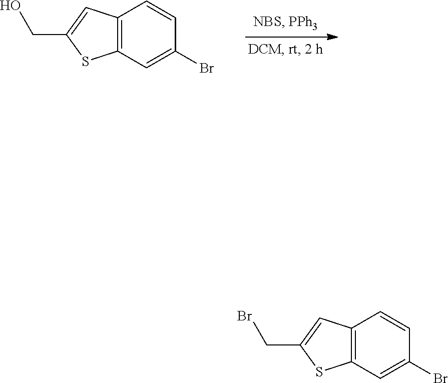 [SMILES: OCc1cc2ccc(Br)cc2s1 ; BrCc1cc2ccc(Br)cc2s1]

To a solution of (6-bromo-1-benzothiophen-2-yl)methanol (2.0 g, 8.22 mmol, 1.0 equiv) in D CM (40 mL), NBS (1.8 g, 9.87 mmol, 1.2 equiv) and PPh₃ (2.6 g, 9.87 mmol, 1.2 equiv) was added in sequence. The mixture was stirred for 2 h at room temperature. Concentrated to remove the solvent, the residue was purified by silica gel column chromatography, eluted with PE/EA (20:1) to afford 6-bromo-2-(bromomethyl)-1-benzothiophene (2.3 g, 91%) as a yellow green solid. LCMS (ES, m/z): [M+H]⁺: 305.

### Synthesis of 3-(6-bromo-1-benzothiophen-2-yl)-2-[(diphenylmethylidene)amino]propanenitrile

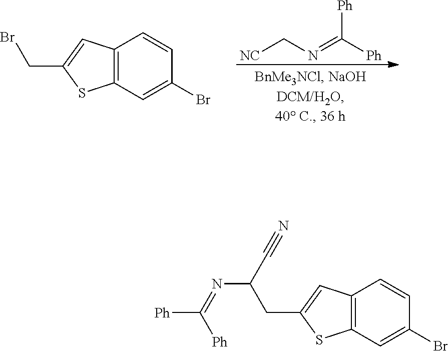 [SMILES: BrCc1cc2ccc(Br)cc2s1 ; N#CCN=C(c1ccccc1)c1ccccc1 ; N#CC(Cc1cc2ccc(Br)cc2s1)N=C(c1ccccc1)c1ccccc1]

To a solution of 6-bromo-2-(bromomethyl)-1-benzothiophene (2.3 g, 7.51 mmol, 1.0 equiv) in DCM (20 mL) was added 2-[(diphenylmethylidene)amino]acetonitrile (1.7 g, 7.53 mmol, 1.0 equiv), benzyltrimethylazanium chloride (0.1 g, 0.75 mmol, 0.1 equiv), NaOH (0.6 g, 15.03 mmol, 2.0 equiv) in H₂O (2 mL) was stirred for 2 h at 40° C. The reaction was diluted with w ater (30 mL), extracted with CH₂Cl₂ (3×100 mL). The combined organic layer was washed with brine (3×30 mL), dried over anhydrous Na₂SO₄. After filtration, the filtrate was concentrated under reduced pressure. The residue was purified by silica gel column chromatography, eluted with PE/THF (5:1) to afford 3-(6-bromo-1-benzothiophen-2-yl)-2-[(diphenylmethylidene)amino]propanenitrile (1.3 g, 39%) as a white solid. LCMS (ES, m/z): [M+H]⁺: 445.

### Synthesis of 2-[(diphenylmethylidene)amino]-3-[6-(3-methyl-2-oxo-1,3-benzoxazol-5-yl)-1-benzothiophen-2-yl]propanenitrile

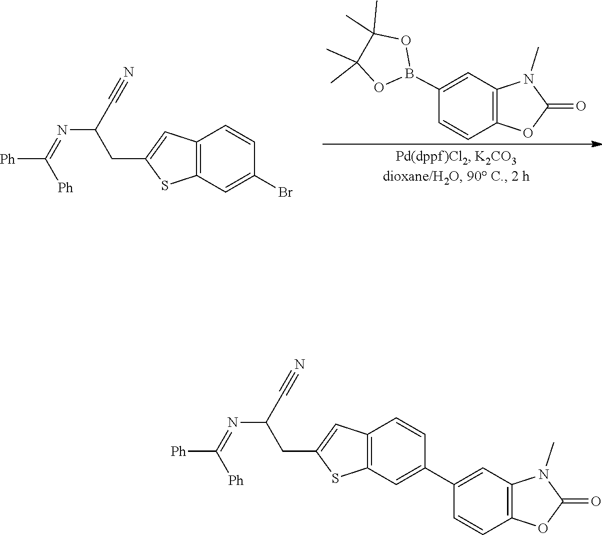 [SMILES: N#CC(Cc1cc2ccc(Br)cc2s1)N=C(c1ccccc1)c1ccccc1 ; Cn1c(=O)oc2ccc(B3OC(C)(C)C(C)(C)O3)cc21 ; Cn1c(=O)oc2ccc(-c3ccc4cc(CC(C#N)N=C(c5ccccc5)c5ccccc5)sc4c3)cc21]

To a solution of 3-(6-bromo-1-benzothiophen-2-yl)-2-[(diphenylmethylidene)amino]propane nitrile (700 mg, 1.57 mmol, 1.0 equiv) and 3-methyl-5-(4,4,5,5-tetramethyl-1,3,2-dioxaborolan-2-yl)-1,3-benzoxazol-2-one (518 mg, 1.88 mmol, 1.2 equiv) in 1,4-dioxane (10 mL) and H₂O (1 mL), K₂CO₃ (434 mg, 3.14 mmol, 2.0 equiv) and Pd(dppf)Cl₂ (115 mg, 0.15 mmol, 0.1 equiv) were added in sequence. The mixture was stirred for 2 h at 90° C. under nitrogen atmosphere. The reaction was cooled to room temperature, concentrated to remove the solvent, the residue was purified by silica gel column chromatography, eluted with PE/THF (1:1) to afford 2-[(diphenylmethylidene)amino]-3-[6-(3-methyl-2-oxo-1,3-benzoxazol-5-yl)-1-benzothiophen-2-yl]propanenitrile (800 mg, 99%) as a yellow oil. LCMS (ES, m/z): [M+H]⁺: 514.

### Synthesis of 2-amino-3-[6-(3-methyl-2-oxo-1,3-benzoxazol-5-yl)-1-benzothiophen-2-yl]propanenitrile

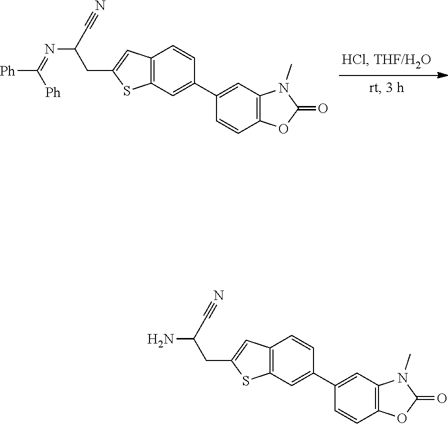 [SMILES: Cn1c(=O)oc2ccc(-c3ccc4cc(CC(C#N)N=C(c5ccccc5)c5ccccc5)sc4c3)cc21 ; Cn1c(=O)oc2ccc(-c3ccc4cc(CC(N)C#N)sc4c3)cc21]

Into a 100 mL round-bottom flask were added 2-[(diphenylmethylidene)amino]-3-[6-(3-methyl-2-oxo-1,3-benzoxazol-5-yl)-1-benzothiophen-2-yl]propanenitrile (400 mg, 0.77 mmol, 1.0 equiv), THE (25 mL), H₂O (2.5 mL) and HCl (1M) (1 mL) were added in sequence at room temperature. The resulting mixture was stirred for additional 3 h at room temperature. The reaction was diluted with water (50 mL), extracted with Et₂O (50 mL). The aqueous phase was basified to pH=8 with Na₂CO₃ solid, extracted with CH₂Cl₂ (3×50 mL). The combined organic layer was washed with brine (3×50 mL), dried over anhydrous Na₂SO₄. After filtration, the filtrate was concentrated under reduced pressure. This resulted in 2-amino-3-[6-(3-methyl-2-oxo-1,3-benzoxazol-5-yl)-1-benzothiophen-2-yl]propanenitrile (140 mg, 51%) as a white solid. LCMS (ES, m/z): [M+H]⁺: 350.

### Synthesis of tert-butyl (2S)-2-({1-cyano-2-[6-(3-methyl-2-oxo-1,3-benzoxazol-5-yl)-1-benzothiophen-2-yl]ethyl}carbamoyl)-1,4-oxazepane-4-carboxylate

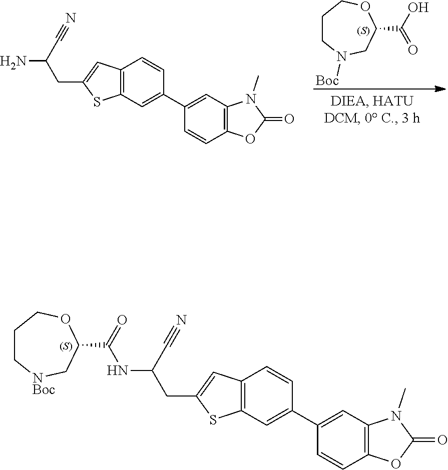

To a solution of 2-amino-3-[6-(3-methyl-2-oxo-1,3-benzoxazol-5-yl)-1-benzothiophen-2-yl]propanenitrile (140 mg, 0.40 mmol, 1.2 equiv) in DCM (5 mL) was treated with (2S)-4-(tert-butoxycarbonyl)-1,4-oxazepane-2-carboxylic acid (82 mg, 0.33 mmol, 1.0 equiv), DIEA (129 mg, 1.00 mmol, 3.0 equiv) followed by the addition of HATU (152 mg, 0.40 mmol, 1.2 equiv) in portions at 0° C. The resulting mixture was stirred for additional 3 h at 0° C. Concentrated to remove the solvent, the residue was purified by silica gel column chromatography, eluted with PE/THF (1:1) to afford tert-butyl (2S)-2-({1-cyano-2-[6-(3-methyl-2-oxo-1,3-benzoxazol-5-yl)-1-benzothiophen-2-yl]ethyl}carbamoyl)-1,4-oxazepane-4-carboxylate (190 mg, 98%) as a white solid. LCMS (ES, m/z): [M+H]⁺: 577.

### Synthesis of (2S)—N-(1-cyano-2-(6-(3-methyl-2-oxo-2,3-dihydrobenzo[d]oxazol-5-yl)benzo[b]thiophen-2-yl)ethyl)-1,4-oxazepane-2-carboxamide

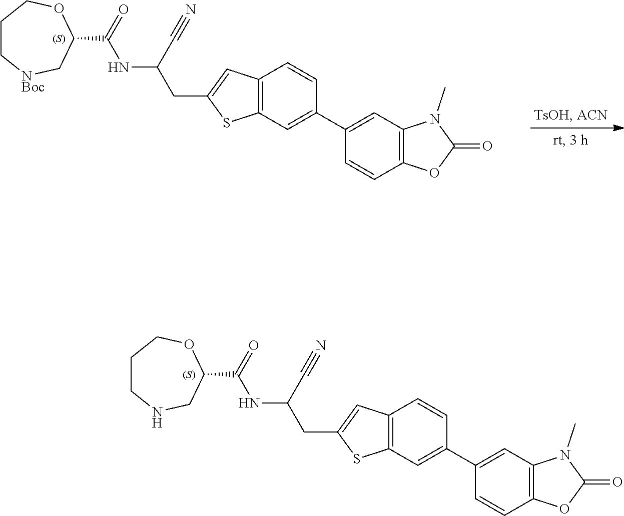

Into a 50 mL round-bottom flask were added tert-butyl (2S)-2-({1-cyano-2-[6-(3-methyl-2-oxo-1,3-benzoxazol-5-yl)-1-benzothiophen-2-yl]ethyl}carbamoyl)-1,4-oxazepane-4-carboxylate (100 mg, 0.17 mmol, 1.0 equiv), TsOH (90 mg, 0.51 mmol, 3.0 equiv) and ACN (3 mL) at room temperature. The resulting mixture was stirred for additional 3 h at room temperature. The reaction solution was purified by reversed-phase flash chromatography with the following conditions: column, C18-120 g, mobile phase, MeCN in Water (0.1% NH₃·H₂O), 10% to 80% gradient in 10 min; detector, UV 254 nm. The fraction was freezing dried, this resulted in (2S)—N-(1-cyano-2-(6-(3-methyl-2-oxo-2,3-dihydrobenzo[d]oxazol-5-yl)benzo[b]thiophen-2-yl)ethyl)-1,4-oxazepane-2-carboxamide (16.3 mg, 20%) as a white solid.

### Analytical Data

LCMS (ES, m/z): [M+H]⁺: 477.

¹H NMR (400 MHz, DMSO-d₆) δ 8.74 (d, J=8.2 Hz, 1H), 8.28 (s, 1H), 7.88 (dd, J=8.3, 2.5 Hz, 1H), 7.72 (d, J=8.3 Hz, 1H), 7.69 (d, J=1.8 Hz, 1H), 7.51 (dd, J=8.7, 1.7 Hz, 1H), 7.42 (d, J=8.3 Hz, 1H), 7.35 (s, 1H), 5.14-5.04 (m, 1H), 4.07-3.95 (m, 1H), 3.95-3.83 (m, 1H), 3.77-3.72 (m, 1H), 3.59-3.51 (m, 2H), 3.43 (s, 3H), 3.20-3.03 (m, 1H), 2.88-2.59 (m, 3H), 1.81-1.72 (m, 2H).

### Example 11. Synthesis of compound 114a: (2S)—N-{1-cyano-2-[5-(3-methyl-2-oxo-1,3-benzoxazol-5-yl)-1-benzothiophen-2-yl]ethyl}-1,4-oxazepane-2-carboxamide

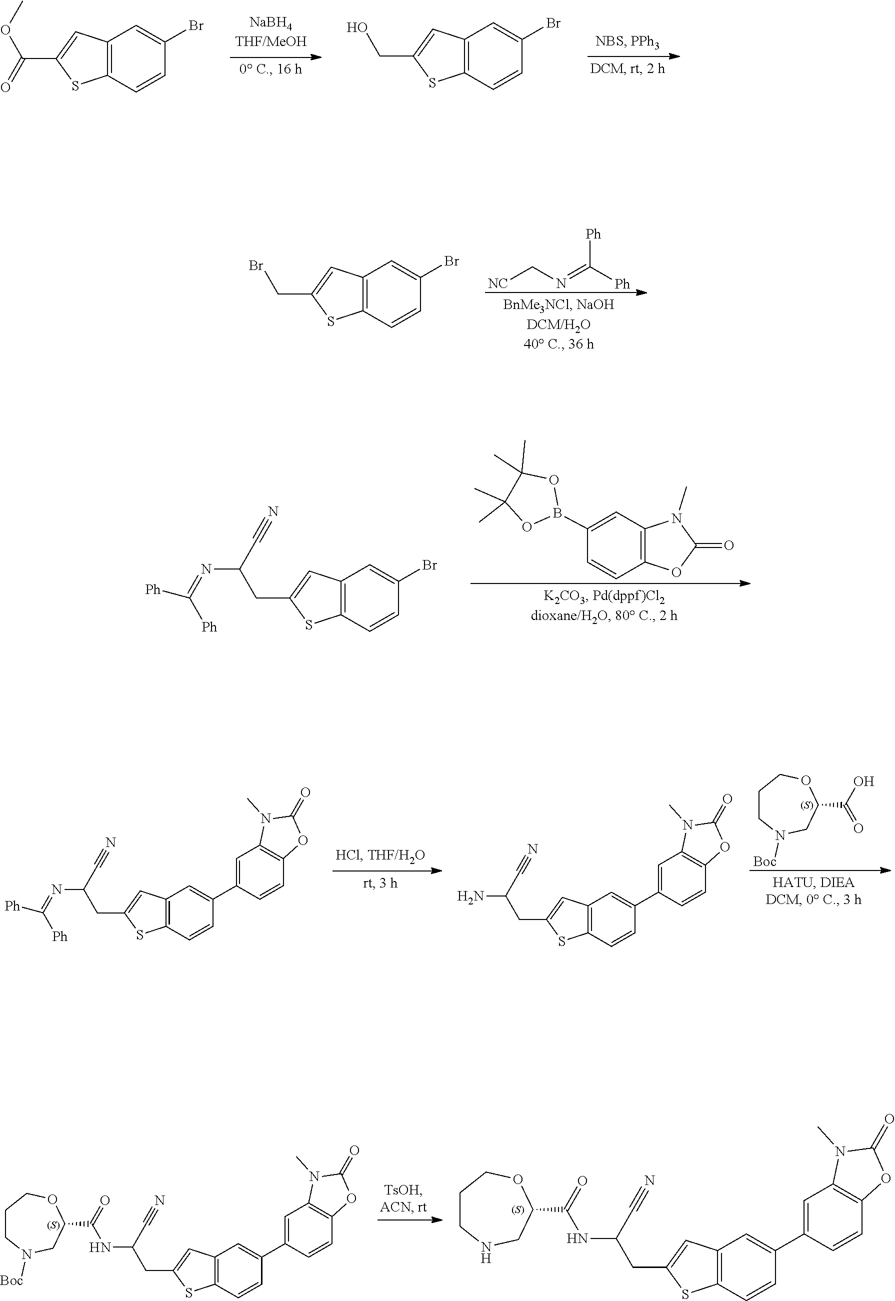

### Synthesis of (5-bromo-1-benzothiophen-2-yl)methanol

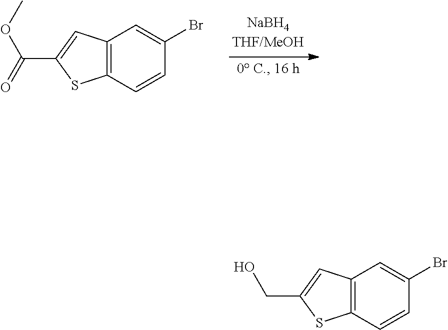

A solution of methyl 5-bromo-1-benzothiophene-2-carboxylate (3.0 g, 11.06 mmol, 1.0 equiv) and NaBH₄ (0.84 g, 22.130 mmol, 2.0 equiv) in MeOH (20 mL) and THE (40 mL) was stirred for 16 h at 0° C. The resulting mixture was extracted with EtOAc (3×100 mL). The combined organic layers were washed with brine (3×30 mL), dried over anhydrous Na₂SO₄. After filtration, the filtrate was concentrated under reduced pressure. The residue was purified by silica gel column chromatography, eluted with PE/THF (5:1) to afford (5-bromo-1-benzothiophen-2-yl)methanol (2.7 g, crude) as a yellow green solid.

¹H NMR (300 MHz, DMSO-d₆) δ 8.01 (d, J=2.0 Hz, 1H), 7.90 (d, J=8.5 Hz, 1H), 7.44 (dd, J=8.5, 2.0 Hz, 1H), 7.25 (q, J=1.0 Hz, 1H), 5.72 (t, J=5.8 Hz, 1H), 4.76 (dd, J=5.8, 1.2 Hz, 2H).

### Synthesis of 5-bromo-2-(bromomethyl)-1-benzothiophene

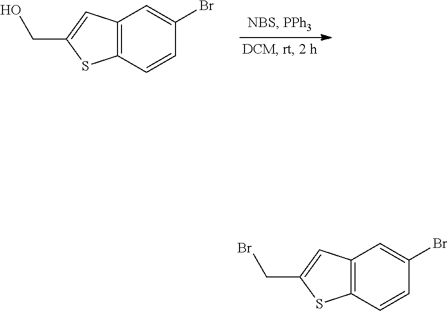 [SMILES: OCc1cc2cc(Br)ccc2s1 ; BrCc1cc2cc(Br)ccc2s1]

A solution of (5-bromo-1-benzothiophen-2-yl)methanol (2.0 g, 8.22 mmol, 1.0 equiv), NBS (1.8 g, 9.87 mmol, 1.2 equiv) and PPh₃ (2.6 g, 9.87 mmol, 1.2 equiv) in DCM (40 mL) was stirred for 2 h at room temperature. The residue was purified by silica gel column chromatography, eluted with PE/EA (20:1) to afford 5-bromo-2-(bromomethyl)-1-benzothiophene (2.2 g, 87.3%) as a yellow green solid.

### Synthesis of 3-(5-bromo-1-benzothiophen-2-yl)-2-[(diphenylmethylidene)amino]propanenitrile

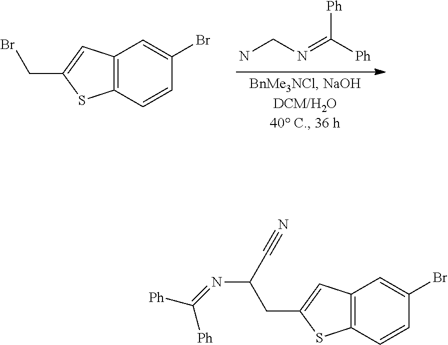

A solution of 5-bromo-2-(bromomethyl)-1-benzothiophene (2.2 g, 7.18 mmol, 1.0 equiv) in DCM (20 mL) was added 2-[(diphenylmethylidene)amino]acetonitrile (1.6 g, 7.18 mmol, 1.0 equiv), benzyltrimethylazanium chloride (0.1 g, 0.71 mmol, 0.1 equiv), NaOH (0.6 g, 14.37 mmol, 2.0 equiv) in H₂O (2 mL) was stirred for 2 h at 40° C. The resulting mixture was extracted with CH₂Cl₂ (3×100 mL). The combined organic layers were washed with brine (3×30 mL), dried over anhydrous Na₂SO₄. After filtration, the filtrate was concentrated under reduced pressure. The residue was purified by silica gel column chromatography, eluted with PE/THF (5:1) to afford 3-(5-bromo-1-benzothiophen-2-yl)-2-[(diphenylmethylidene)amino]propanenitrile (2.3 g, 71.8%) as a white solid. LCMS (ES, m/z): [M+H]⁺:445

### Synthesis of 2-[(diphenylmethylidene)amino]-3-[5-(3-methyl-2-oxo-1,3-benzoxazol-5-yl)-1-benzothiophen-2-yl]propanenitrile

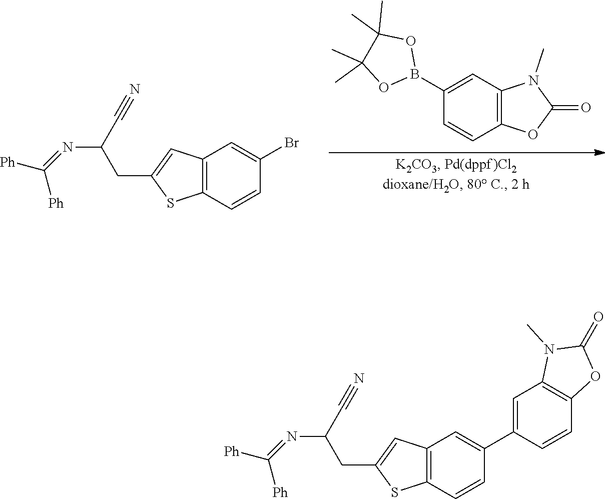 [SMILES: N#CC(Cc1cc2cc(Br)ccc2s1)N=C(c1ccccc1)c1ccccc1 ; Cn1c(=O)oc2ccc(B3OC(C)(C)C(C)(C)O3)cc21 ; Cn1c(=O)oc2ccc(-c3ccc4sc(CC(C#N)N=C(c5ccccc5)c5ccccc5)cc4c3)cc21]

A solution of 3-(5-bromo-1-benzothiophen-2-yl)-2-[(diphenylmethylidene)amino]propanenitrile (1.0 g, 2.24 mmol, 1.0 equiv) and 3-methyl-5-(4,4,5,5-tetramethyl-1,3,2-dioxaborolan-2-yl)-1,3-benzoxazol-2-one (0.7 g, 2.694 mmol, 1.2 equiv), K₂CO₃ (0.6 g, 4.49 mmol, 2.0 equiv), Pd(dppf)Cl₂ (0.2 g, 0.22 mmol, 0.1 equiv) in 1,4-dioxane (10 mL) and H₂O (1 mL) was stirred for 2 h at 90° C. under nitrogen atmosphere. The residue was purified by silica gel column chromatography, eluted with PE/THF (1:1) to afford 2-[(diphenylmethylidene)amino]-3-[5-(3-methyl-2-oxo-1,3-benzoxazol-5-yl)-1-benzothiophen-2-yl]propanenitrile (1.1 g, 95.3%) as a yellow solid. LCMS (ES, m/z): [M+H]⁺:514

### Synthesis of 2-amino-3-[5-(3-methyl-2-oxo-1,3-benzoxazol-5-yl)-1-benzothiophen-2-yl]propanenitrile

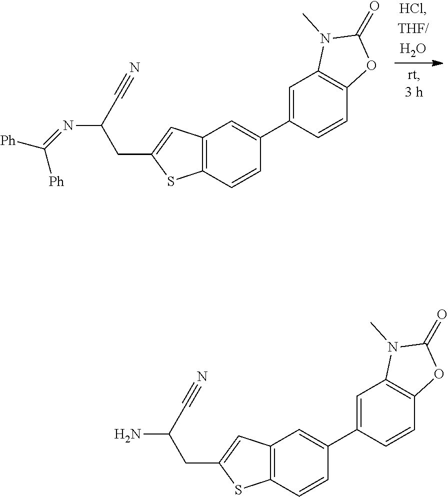 [SMILES: Cn1c(=O)oc2ccc(-c3ccc4sc(CC(C#N)N=C(c5ccccc5)c5ccccc5)cc4c3)cc21 ; Cn1c(=O)oc2ccc(-c3ccc4sc(CC(N)C#N)cc4c3)cc21]

Into a 100 mL round-bottom flask were added 2-[(diphenylmethylidene)amino]-3-[5-(3-methyl-2-oxo-1,3-benzoxazol-5-yl)-1-benzothiophen-2-yl]propanenitrile (500 mg, 0.97 mmol, 1.0 equiv) and HCl (1M) (1 mL), THE (30 mL) and H₂O (3 mL) at room temperature. The resulting mixture was stirred for additional 3 h at room temperature. The resulting mixture was extracted with Et₂O (1×50 mL). The organic layers were extracted with CH₂Cl₂ (3×100 mL). The combined organic layers were washed with brine (3×30 mL), dried over anhydrous Na₂SO₄. After filtration, the filtrate was concentrated under reduced pressure. This resulted in 2-amino-3-[5-(3-methyl-2-oxo-1,3-benzoxazol-5-yl)-1-benzothiophen-2-yl]propanenitrile (220 mg, 64.6%) as a white solid. LCMS (ES, m/z): [M+H]⁺:350

### Synthesis of (2S)—N-{1-cyano-2-[5-(3-methyl-2-oxo-1,3-benzoxazol-5-yl)-1-benzothiophen-2-yl]ethyl}-1,4-oxazepane-2-carboxamide

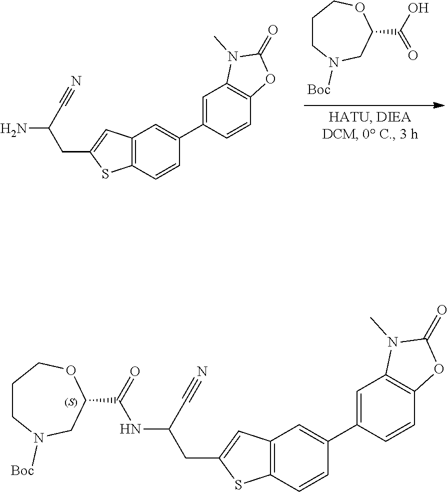

A solution of 2-amino-3-[5-(3-methyl-2-oxo-1,3-benzoxazol-5-yl)-1-benzothiophen-2-yl]propanenitrile (156 mg, 0.44 mmol, 1.1 equiv) in DCM (5 mL) was treated with (2S)-4-(tert-butoxycarbonyl)-1,4-oxazepane-2-carboxylic acid (100 mg, 0.40 mmol, 1.0 equiv), DIEA (158 mg, 1.22 mmol, 3.0 equiv) followed by the addition of HATU (186 mg, 0.49 mmol, 1.2 equiv) in portions at 0° C. The resulting mixture was stirred for additional 2 h at 0° C. The residue was purified by silica gel column chromatography, eluted with PE/THF (1:1) to afford (2S)—N-{1-cyano-2-[5-(3-methyl-2-oxo-1,3-benzoxazol-5-yl)-1-benzothiophen-2-yl]ethyl}-1,4-oxazepane-2-carboxamide (220 mg, crude) as a white solid. LCMS (ES, m/z): [M+H]⁺:577

### Synthesis of (2S)—N-{1-cyano-2-[5-(3-methyl-2-oxo-1,3-benzoxazol-5-yl)-1-benzothiophen-2-yl]ethyl}-1,4-oxazepane-2-carboxamide

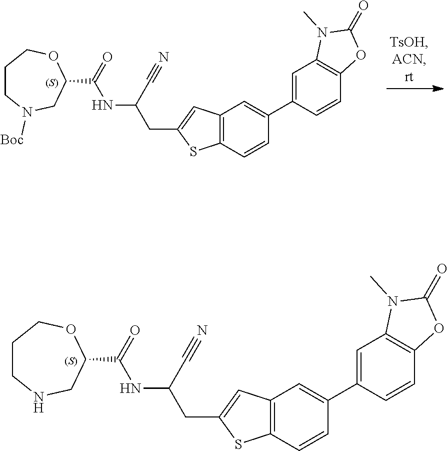

Into a 8 mL vial were added tert-butyl (2S)-2-({1-cyano-2-[5-(3-methyl-2-oxo-1,3-benzoxazol-5-yl)-1-benzothiophen-2-yl]ethyl}carbamoyl)-1,4-oxazepane-4-carboxylate (100 mg, 0.17 mmol, 1.0 equiv), TsOH (89 mg, 0.51 mmol, 3.0 equiv) and ACN (3 mL) at room temperature. The resulting mixture was stirred for additional 3 h at room temperature. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in (2S)—N-{1-cyano-2-[5-(3-methyl-2-oxo-1,3-benzoxazol-5-yl)-1-benzothiophen-2-yl]ethyl}-1,4-oxazepane-2-carboxamide (21.9 mg, 26.50%) as a white solid.

### Analytical Data

LCMS (ES, m/z): [M+H]⁺:477.2.

¹H NMR (400 MHz, DMSO-d₆) δ 8.73 (d, J=8.3 Hz, 1H), 8.11 (t, J=2.3 Hz, 1H), 8.06-7.98 (m, 1H), 7.71-7.63 (m, 2H), 7.49 (d, J=8.4 Hz, 1H), 7.43 (d, J=8.3 Hz, 1H), 7.38 (s, 1H), 5.10 (dq, J=15.4, 8.2 Hz, 1H), 4.00 (ddd, J=25.9, 7.9, 3.6 Hz, 1H), 3.94-3.84 (m, 1H), 3.81-3.67 (m, 1H), 3.62-3.46 (m, 2H), 3.43 (s, 3H), 3.15 (dd, J=14.2, 3.7 Hz, 1H), 3.06 (dd, J=14.2, 3.6 Hz, 1H), 2.85-2.67 (m, 2H), 2.60 (dd, J=14.4, 8.1 Hz, 1H), 1.81-1.67 (m, 2H).

### Example 12. Synthesis of compound 115a: (2S)—N-{1-cyano-2-[3-fluoro-5-(3-methyl-2-oxo-1,3-benzoxazol-5-yl)-1-benzothiophen-2-yl]ethyl}-1,4-oxazepane-2-carboxamide

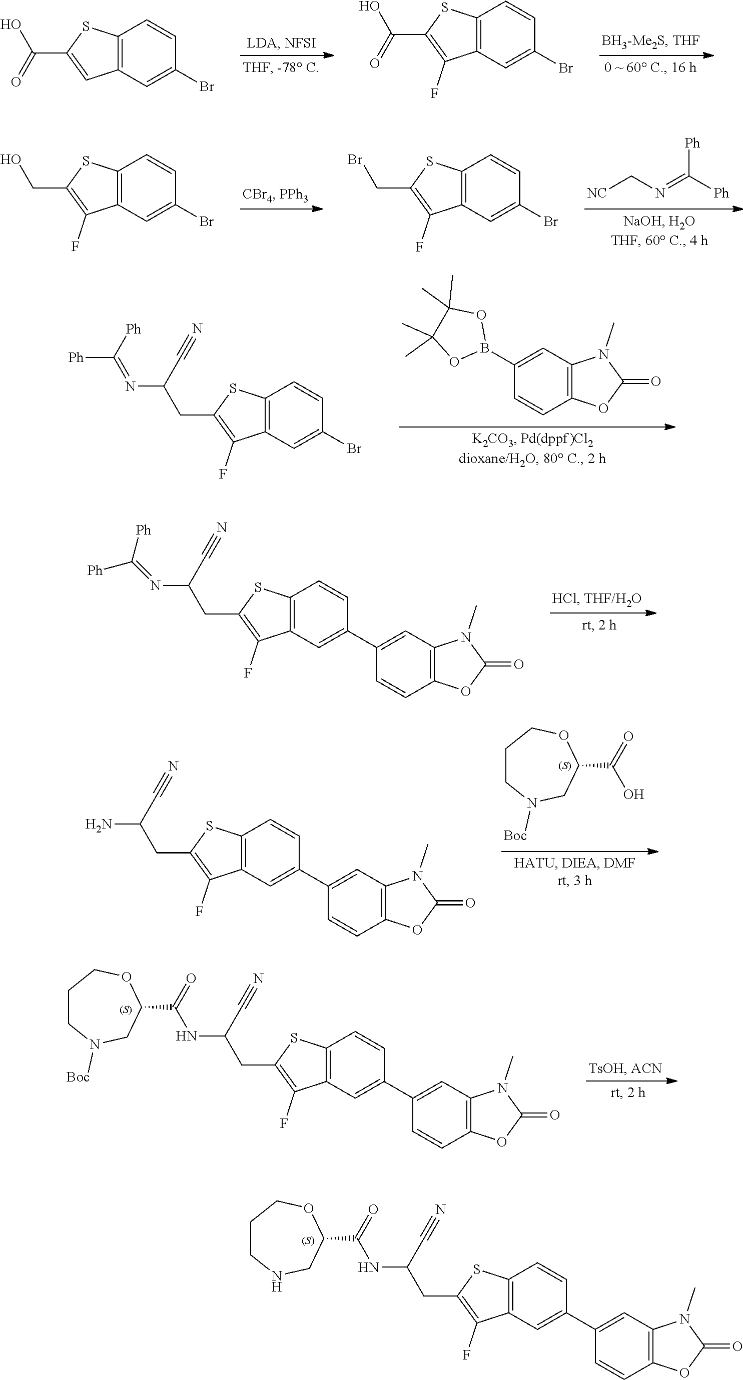

### Synthesis of 5-bromo-3-fluoro-1-benzothiophene-2-carboxylic acid

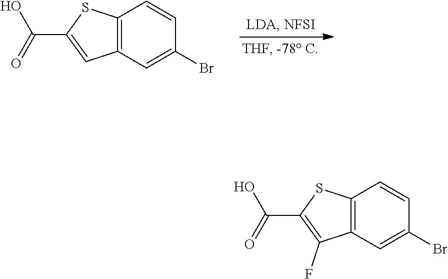 [SMILES: O=C(O)c1cc2cc(Br)ccc2s1 ; O=C(O)c1sc2ccc(Br)cc2c1F]

To a stirred solution of 5-bromo-1-benzothiophene-2-carboxylic acid (10 g, 38.90 mmol, 1 equiv) in THF (300 mL) was added LDA (in 2M THF) (97 mL, 194 mmol, 5 equiv) dropwise at −78° C. under nitrogen atmosphere. The resulting mixture was stirred for 1 h at −78° C. under nitrogen atmosphere. To the above mixture was added NFSI (61.32 g, 194 mmol, 5 equiv) in THF (300 mL) dropwise over 30 min at −78° C. The resulting mixture was stirred for additional 16 h at room temperature. The reaction was quenched with Water at room temperature. The mixture was acidified to pH 7-8 with saturated NaHCO₃ (aq.). The resulting mixture was extracted with EtOAc (3×100 mL). The combined water layers were acidified to pH 4 with conc. HCl. The precipitated solids were collected by filtration and washed with H₂O (3×60 mL). This resulted in 5-bromo-3-fluoro-1-benzothiophene-2-carboxylic acid (7 g, 65.42%) as an off-white solid. LCMS (ES, m/z): [M−H]—: 273.

### Synthesis of (5-bromo-3-fluoro-1-benzothiophen-2-yl)methanol

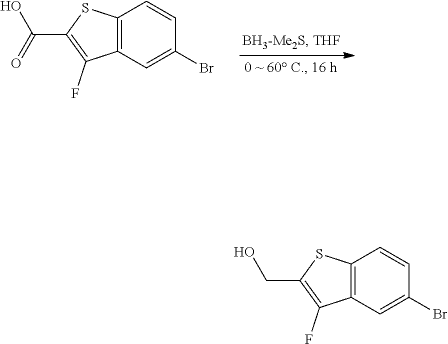 [SMILES: O=C(O)c1sc2ccc(Br)cc2c1F ; OCc1sc2ccc(Br)cc2c1F]

To a stirred solution of 5-bromo-3-fluoro-1-benzothiophene-2-carboxylic acid (1.5 g, 5.45 mmol, 1 equiv) and THF (30 mL) was added BH₃-Me₂S (1.24 g, 16.36 mmol, 3 equiv) dropwise at 0° C. The resulting mixture was stirred for 16 h at 60° C. The reaction was quenched with MeOH at room temperature. The resulting mixture was extracted with EtOAc (3×50 mL). The resulting mixture was concentrated under reduced pressure. The residue was purified by silica gel column chromatography, eluted with PE/EA (5:1) to afford (5-bromo-3-fluoro-1-benzothiophen-2-yl)methanol (1 g, 70.24%) as an off-white solid.

¹H NMR (400 MHz, DMSO-d₆) δ 7.96 (dd, J=8.6, 1.9 Hz, 1H), 7.92 (d, J=2.0 Hz, 1H), 7.58 (dd, J=8.6, 2.0 Hz, 1H), 5.77 (t, J=5.8 Hz, 1H), 4.74 (dd, J=5.8, 1.8 Hz, 2H).

### Synthesis of 5-bromo-2-(bromomethyl)-3-fluoro-1-benzothiophene

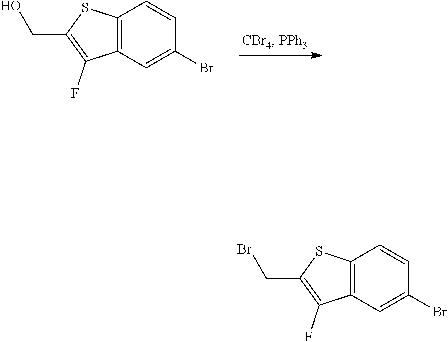 [SMILES: OCc1sc2ccc(Br)cc2c1F ; Fc1c(CBr)sc2ccc(Br)cc12]

To a stirred solution of (5-bromo-3-fluoro-1-benzothiophen-2-yl)methanol (950 mg, 3.64 mmol, 1 equiv) and NBS (777 mg, 4.37 mmol, 1.2 equiv) in DCM (20 mL) was added PPh₃ (1145 mg, 4.37 mmol, 1.2 equiv) at room temperature under nitrogen atmosphere. The resulting mixture was stirred for 3 h at room temperature under nitrogen atmosphere. The resulting mixture was extracted with CH₂Cl₂ (3×50 mL). The combined organic layers were washed with brine (3×50 mL), dried over anhydrous Na₂SO₄. After filtration, the filtrate was concentrated under reduced pressure. This resulted in 5-bromo-2-(bromomethyl)-3-fluoro-1-benzothiophene (1.1 g, 93.31%) as a light-yellow oil.

### Synthesis of 3-(5-bromo-3-fluoro-1-benzothiophen-2-yl)-2-[(diphenylmethylidene)amino]propanenitrile

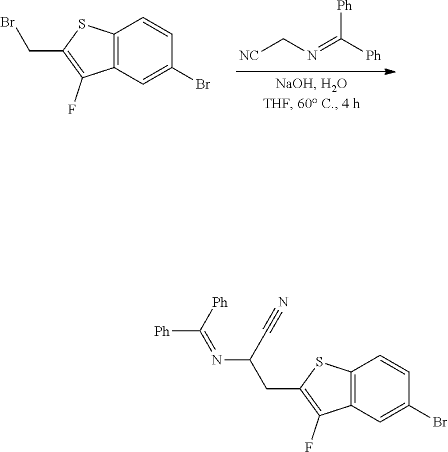 [SMILES: Fc1c(CBr)sc2ccc(Br)cc12 ; N#CCN=C(c1ccccc1)c1ccccc1 ; N#CC(Cc1sc2ccc(Br)cc2c1F)N=C(c1ccccc1)c1ccccc1]

A solution of 5-bromo-2-(bromomethyl)-3-fluoro-1-benzothiophene (1.1 g, 3.40 mmol, 1 equiv) in THE (10 mL) was added 2-[(diphenylmethylidene)amino]acetonitrile (0.75 g, 3.40 mmol, 1 equiv), benzyltrimethylazanium chloride (0.06 g, 0.34 mmol, 0.1 equiv), NaOH (0.27 g, 6.79 mmol, 2 equiv) in H₂O (2 mL) was stirred for 4 h at 60° C. The resulting mixture was extracted with CH₂Cl₂ (3×100 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EA (5:1) to afford 3-(5-bromo-3-fluoro-1-benzothiophen-2-yl)-2-[(diphenylmethylidene)amino]propanenitrile (1.5 g, 95.35%) as an off-white solid. LCMS (ES, m/z): [M+H]⁺: 463.

### Synthesis of 2-[(diphenylmethylidene)amino]-3-[3-fluoro-5-(3-methyl-2-oxo-1,3-benzoxazol-5-yl)-1-benzothiophen-2-yl]propanenitrile

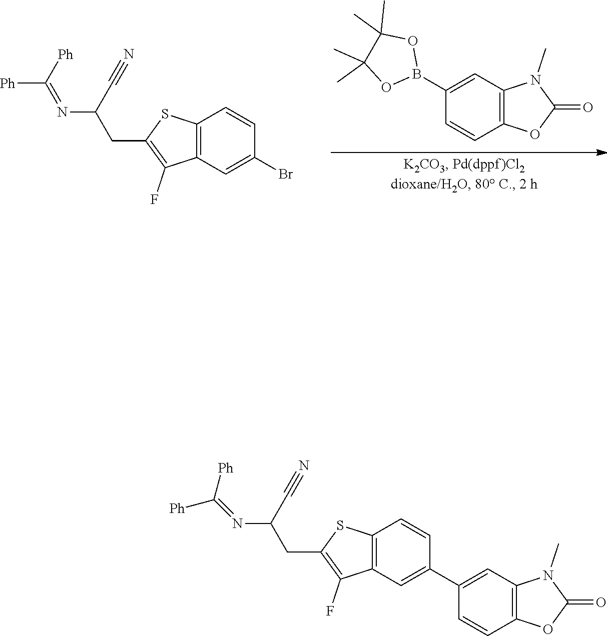 [SMILES: N#CC(Cc1sc2ccc(Br)cc2c1F)N=C(c1ccccc1)c1ccccc1 ; Cn1c(=O)oc2ccc(B3OC(C)(C)C(C)(C)O3)cc21 ; Cn1c(=O)oc2ccc(-c3ccc4sc(CC(C#N)N=C(c5ccccc5)c5ccccc5)c(F)c4c3)cc21]

A solution of 3-(5-bromo-3-fluoro-1-benzothiophen-2-yl)-2-[(diphenylmethylidene)amino]propanenitrile (800 mg, 1.73 mmol, 1 equiv), K₂CO₃ (477 mg, 3.45 mmol, 2 equiv), 3-methyl-5-(4,4,5-trimethyl-1,3,2-dioxaborolan-2-yl)-1,3-benzoxazol-2-one (496 mg, 1.90 mmol, 1.1 equiv) and Pd(dppf)Cl₂ (131 mg, 0.18 mmol, 0.1 equiv) in 1,4-dioxane (10 mL), H₂O (1.5 mL) was stirred for 2 h at 80° C. under nitrogen atmosphere. The residue was purified by silica gel column chromatography, eluted with PE/THF (5:1) to afford 2-[(diphenylmethylidene)amino]-3-[3-fluoro-5-(3-methyl-2-oxo-1,3-benzoxazol-5-yl)-1-benzothiophen-2-yl]propanenitrile (700 mg, 76.27%) as a light yellow oil. LCMS (ES, m/z): [M+H]⁺: 532.

### Synthesis of 2-amino-3-[3-fluoro-5-(3-methyl-2-oxo-1,3-benzoxazol-5-yl)-1-benzothiophen-2-yl]propanenitrile

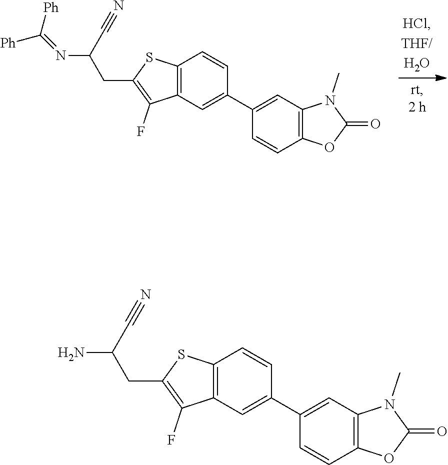 [SMILES: Cn1c(=O)oc2ccc(-c3ccc4sc(CC(C#N)N=C(c5ccccc5)c5ccccc5)c(F)c4c3)cc21 ; Cn1c(=O)oc2ccc(-c3ccc4sc(CC(N)C#N)c(F)c4c3)cc21]

Into a 100 mL round-bottom flask were added 2-[(diphenylmethylidene)amino]-3-[3-fluoro-5-(3-methyl-2-oxo-1,3-benzoxazol-5-yl)-1-benzothiophen-2-yl]propanenitrile (700 mg, 1.32 mmol, 1 equiv) and THE (30 mL), H₂O (3 mL), HCl (2 mL, 10.00 mmol, 7.59 equiv) at room temperature. The resulting mixture was stirred for additional 3 h at room temperature. The mixture was basified to pH 12 with NaOH. The resulting mixture was extracted with EtOAc (3×50 mL). The combined organic layers were washed with brine (3×30 mL), dried over anhydrous Na₂SO₄. After filtration, the filtrate was concentrated under reduced pressure. The residue was purified by silica gel column chromatography, eluted with PE/THF (1:2) to afford 2-amino-3-[3-fluoro-5-(3-methyl-2-oxo-1,3-benzoxazol-5-yl)-1-benzothiophen-2-yl]propanenitrile (350 mg, 72.35%) as a white solid. LCMS (ES, m/z): [M+H]⁺: 368.

### Synthesis of tert-butyl (2S)-2-({1-cyano-2-[3-fluoro-5-(3-methyl-2-oxo-1,3-benzoxazol-5-yl)-1-benzothiophen-2-yl]ethyl}carbamoyl)-1,4-oxazepane-4-carboxylate

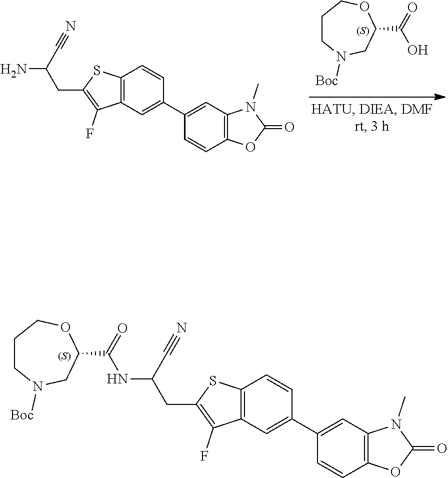

To a stirred mixture of 2-amino-3-[3-fluoro-5-(3-methyl-2-oxo-1,3-benzoxazol-5-yl)-1-benzothiophen-2-yl]propanenitrile (99 mg, 0.270 mmol, 1.1 equiv), (2S)-4-(tert-butoxycarbonyl)-1,4-oxazepane-2-carboxylic acid (60 mg, 0.25 mmol, 1.00 equiv) and DIEA (95 mg, 0.74 mmol, 3 equiv) in DCM (5 mL) were added HATU (112 mg, 0.29 mmol, 1.2 equiv) in portions at 0° C. The resulting mixture was stirred for additional 3 h at 0° C. The residue was purified by silica gel column chromatography, eluted with PE/THF (1:1) to afford tert-butyl (2S)-2-({1-cyano-2-[3-fluoro-5-(3-methyl-2-oxo-1,3-benzoxazol-5-yl)-1-benzothiophen-2-yl]ethyl}carbamoyl)-1,4-oxazepane-4-carboxylate (120 mg, 82.49%) as alight yellow oil. LCMS (ES, m/z): [M+H]⁺: 595.

### Synthesis of (2S)—N-{1-cyano-2-[3-fluoro-5-(3-methyl-2-oxo-1,3-benzoxazol-5-yl)-1-benzothiophen-2-yl]ethyl}-1,4-oxazepane-2-carboxamide

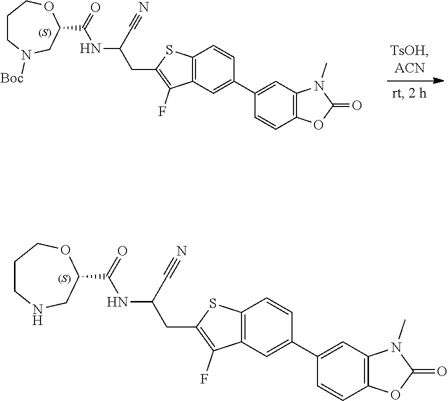

Into a 50 mL round-bottom flask were added tert-butyl (2S)-2-({1-cyano-2-[3-fluoro-5-(3-methyl-2-oxo-1,3-benzoxazol-5-yl)-1-benzothiophen-2-yl]ethyl}carbamoyl)-1,4-oxazepane-4-carboxylate (120 mg, 0.20 mmol, 1 equiv), TsOH (108 mg, 0.62 mmol, 3.0 equiv) and ACN (3 mL) at room temperature. The resulting mixture was stirred for additional 3 h at room temperature. The residue was purified by reversed-phase flash chromatography with the following conditions: column, C₁₈ silica gel; mobile phase, MeCN in Water (0.1% NH₃·H₂O), 10% to 50% gradient in 10 min; detector, UV 254 nm. This resulted in (2S)—N-{1-cyano-2-[3-fluoro-5-(3-methyl-2-oxo-1,3-benzoxazol-5-yl)-1-benzothiophen-2-yl]ethyl}-1,4-oxazepane-2-carboxamide (20 mg, 20.04%) as a white solid.

### Analytical Data

LCMS (ES, m/z): [M+H]⁺: 495.

¹H NMR (400 MHz, DMSO-d₆) ¹H NMR (300 MHz, DMSO-d₆) δ 8.81 (d, J=8.5 Hz, 1H), 8.12-8.01 (m, 2H), 7.81 (d, J=8.5 Hz, 1H), 7.74 (t, J=1.8 Hz, 1H), 7.59-7.49 (m, 1H), 7.44 (d, J=8.3 Hz, 1H), 5.14-5.03 (m, 1H), 4.07-3.82 (m, 2H), 3.74 (td, J=7.8, 3.5 Hz, 1H), 3.58 (dd, J=14.5, 7.0 Hz, 1H), 3.51-3.43 (m, 1H), 3.44 (s, 3H), 3.22-3.02 (m, 1H), 2.88-2.58 (m, 4H), 1.78-1.67 (m, 2H).

### Example 13. Synthesis of compound 130a: (2S)—N-{1-cyano-2-[3-fluoro-6-(3-methyl-2-oxo-1,3-benzoxazol-5-yl)-1-benzothiophen-2-yl]ethyl}-1,4-oxazepane-2-carboxamide

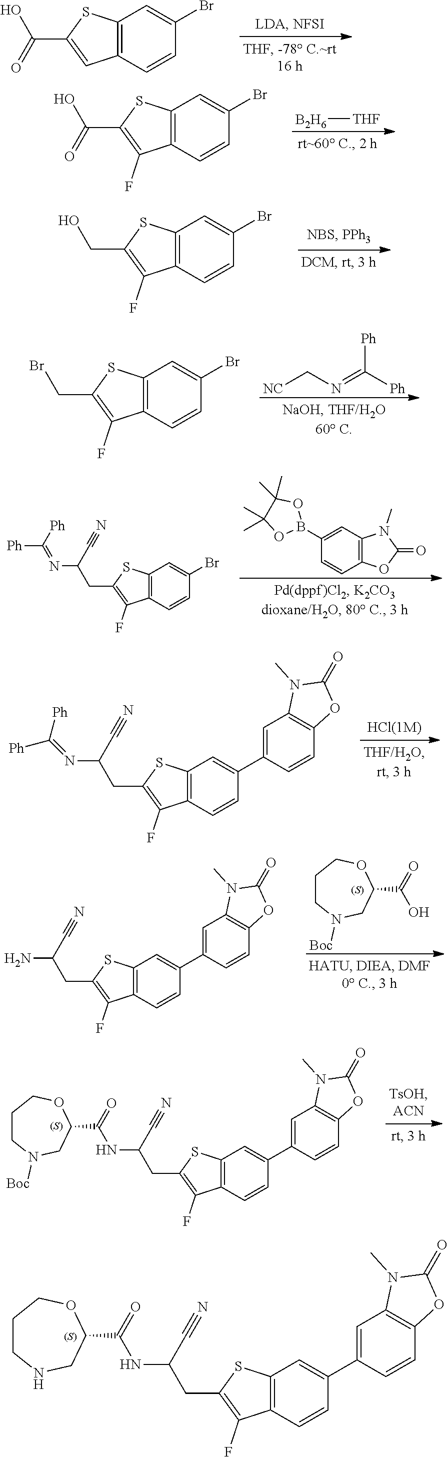

### Synthesis of 6-bromo-3-fluoro-1-benzothiophene-2-carboxylic acid

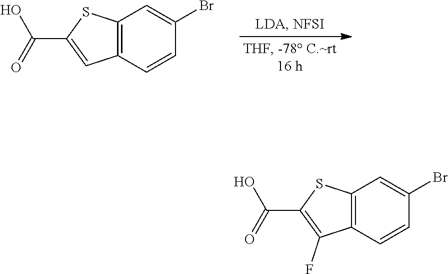 [SMILES: O=C(O)c1cc2ccc(Br)cc2s1 ; O=C(O)c1sc2cc(Br)ccc2c1F]

To a solution of 6-bromo-1-benzothiophene-2-carboxylic acid (5.5 g, 21.39 mmol, 1.0 equiv) in THF (100 mL) was added LDA (2 M in THF) (53.5 mL, 106.96 mmol, 5.0 equiv) dropwise at −78° C. under nitrogen atmosphere. After addition, the mixture was stirred for 1 h, this was followed by the addition of NFSI (33.7 g, 106.96 mmol, 5.0 equiv) in THF (50 mL) dropwise. The reaction was warmed to room temperature slowly and stirred for 12 h. The reaction was quenched with HCl (aq) (200 mL), extracted with ethyl acetate (200 mL×2). The combined organic phase was dried over anhydrous sodium sulfate, filtered and the filtrate was concentrated, this result in 6-bromo-3-fluoro-1-benzothiophene-2-carboxylic acid (1.9 g, 32%) as yellow solid and used to the next step without further purification.

### Synthesis of (6-bromo-3-fluoro-1-benzothiophen-2-yl)methanol

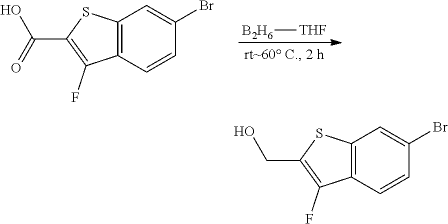

To a solution of 6-bromo-3-fluoro-1-benzothiophene-2-carboxylic acid (1.9 g, 6.90 mmol, 1.0 equiv) in THF (30 mL) was added B2H₆ (1 M in THF) at room temperature under nitrogen atmosphere. The reaction was heated to 60° C. and stirred for 3 h. The reaction was cooled to room temperature, quenched with HCl (1 N) (20 mL), diluted with water (30 mL), extracted with ethyl acetate (50 mL×2). The combined organic phase was dried over anhydrous sodium sulfate. Filtered and the filtrate was concentrated and purified by silica gel column with ethyl acetate/petroleum ether 5~10%, this result in (6-bromo-3-fluoro-1-benzothiophen-2-yl)methanol (1 g, 55.4%) as white solid. LCMS (ES, m/z): [M−H₂O+H]⁺: 243.

### Synthesis of 6-bromo-2-(bromomethyl)-3-fluoro-1-benzothiophene

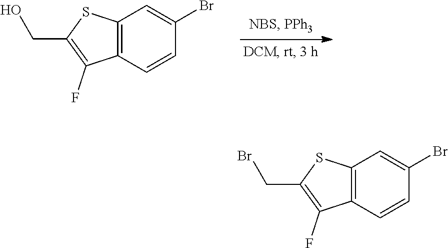 [SMILES: OCc1sc2cc(Br)ccc2c1F ; Fc1c(CBr)sc2cc(Br)ccc12]

A solution of (6-bromo-3-fluoro-1-benzothiophen-2-yl)methanol (1.0 g, 3.83 mmol, 1.0 equiv) and NBS (0.8 g, 4.59 mmol, 1.2 equiv) in DCM (10 mL) was stirred for 3 h at room temperature under nitrogen atmosphere. Concentrated to remove the solvent, the residue was purified by silica gel column chromatography, eluted with PE/EA (10:1) to afford 6-bromo-2-(bromomethyl)-3-fluoro-1-benzothiophene (1.0 g, 80.5%) as white solid (no LCMS signal).

### Synthesis of 3-(6-bromo-3-fluoro-1-benzothiophen-2-yl)-2-[(diphenylmethylidene)amino]propanenitrile

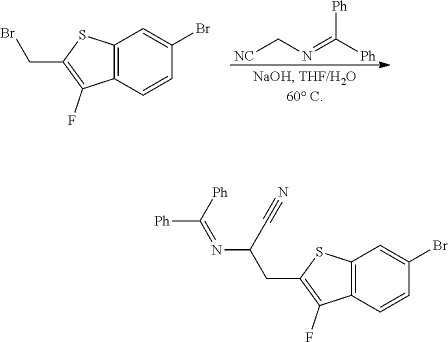 [SMILES: Fc1c(CBr)sc2cc(Br)ccc12 ; N#CCN=C(c1ccccc1)c1ccccc1 ; N#CC(Cc1sc2cc(Br)ccc2c1F)N=C(c1ccccc1)c1ccccc1]

A solution of 6-bromo-2-(bromomethyl)-3-fluoro-1-benzothiophene (1.0 g, 3.08 mmol, 1.0 equiv) in THE (10 mL) was added 2-[(diphenylmethylidene)amino]acetonitrile (0.7 g, 3.08 mmol, 1.0 equiv), benzyltrimethylazanium chloride (0.1 g, 0.30 mmol, 0.1 equiv), NaOH (0.3 g, 6.17 mmol, 2.0 equiv) in H₂O (2 mL) was stirred for 2 h at 60° C. The resulting mixture was diluted with water (20 mL), extracted with EtOAc (3×50 mL). The combined organic layer was washed with brine (3×30 mL), dried over anhydrous Na₂SO₄. After filtration, the filtrate was concentrated under reduced pressure. The residue was purified by silica gel column chromatography, eluted with PE/THF (5:1) to afford 3-(6-bromo-3-fluoro-1-benzothiophen-2-yl)-2-[(diphenylmethylidene)amino]propanenitrile (1.3 g, 90.9%) as yellow solid. LCMS (ES, m/z): [M+H]⁺: 463.

### Synthesis of 2-[(diphenylmethylidene)amino]-3-[3-fluoro-6-(3-methyl-2-oxo-1,3-benzoxazol-5-yl)-1-benzothiophen-2-yl]propanenitrile

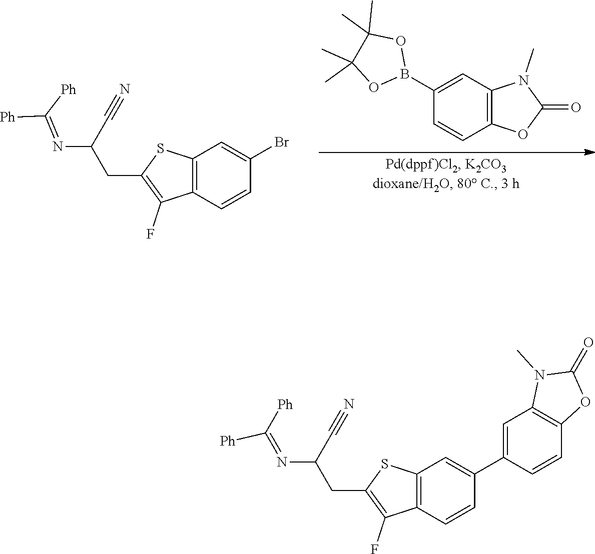 [SMILES: N#CC(Cc1sc2cc(Br)ccc2c1F)N=C(c1ccccc1)c1ccccc1 ; Cn1c(=O)oc2ccc(B3OC(C)(C)C(C)(C)O3)cc21 ; Cn1c(=O)oc2ccc(-c3ccc4c(F)c(CC(C#N)N=C(c5ccccc5)c5ccccc5)sc4c3)cc21]

To a mixture of 3-(6-bromo-3-fluoro-1-benzothiophen-2-yl)-2-[(diphenylmethylidene)amino]propanenitrile (1.3 g, 2.80 mmol, 1.1 equiv), 3-methyl-5-(4,4,5,5-tetramethyl-1,3,2-dioxaborolan-2-yl)-1,3-benzoxazol-2-one (0.7 g, 2.55 mmol, 1.0 equiv), in 1,4-dioxane (10 mL) and H₂O (1 mL), K₂CO₃ (0.7 g, 5.10 mmol, 2.0 equiv) and Pd(dppf)Cl₂ (0.2 g, 0.25 mmol, 0.1 equiv) were added in sequence. The reaction was stirred for 3 h at 80° C. under nitrogen atmosphere. The reaction was cooled to room temperature, concentrated to remove the solvent, the residue was purified by silica gel column chromatography, eluted with PE/THF (1:1) to afford 2-[(diphenylmethylidene)amino]-3-[3-fluoro-6-(3-methyl-2-oxo-1,3-benzoxazol-5-yl)-1-benzothiophen-2-yl]propanenitrile (1.3 g, 95.8%) as yellow oil. LCMS (ES, m/z): [M+H]⁺: 532.

### Synthesis of 2-amino-3-[3-fluoro-6-(3-methyl-2-oxo-1,3-benzoxazol-5-yl)-1-benzothiophen-2-yl]propanenitrile

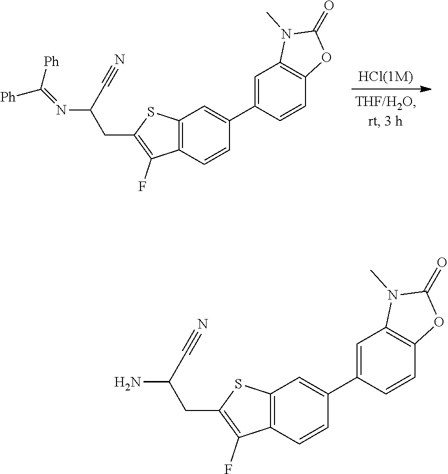 [SMILES: Cn1c(=O)oc2ccc(-c3ccc4c(F)c(CC(C#N)N=C(c5ccccc5)c5ccccc5)sc4c3)cc21 ; Cn1c(=O)oc2ccc(-c3ccc4c(F)c(CC(N)C#N)sc4c3)cc21]

Into a 100 mL round-bottom flask were added 2-[(diphenylmethylidene)amino]-3-[3-fluoro-6-(3-methyl-2-oxo-1,3-benzoxazol-5-yl)-1-benzothiophen-2-yl]propanenitrile (1.3 g, 2.44 mmol, 1.0 equiv) and HCl (1M) (3 mL), H₂O (6 mL) at room temperature. The resulting mixture was stirred for 3 h at room temperature. The resulting mixture was extracted with Et₂O (1×50 mL). The mixture was basified to pH-12 with NaOH (2 M in water). The aqueous layer was extracted with CH₂Cl₂ (3×50 mL). The combined organic layer was washed with brine (3×50 mL), dried over anhydrous Na₂SO₄. After filtration, the filtrate was concentrated under reduced pressure. This resulted in 2-amino-3-[3-fluoro-6-(3-methyl-2-oxo-1,3-benzoxazol-5-yl)-1-benzothiophen-2-yl]propanenitrile (560 mg, 62.3%) as white solid. LCMS (ES, m/z): [M+H]⁺: 367.

### Synthesis of tert-butyl (2S)-2-({1-cyano-2-[3-fluoro-6-(3-methyl-2-oxo-1,3-benzoxazol-5-yl)-1-benzothiophen-2-yl]ethyl}carbamoyl)-1,4-oxazepane-4-carboxylate

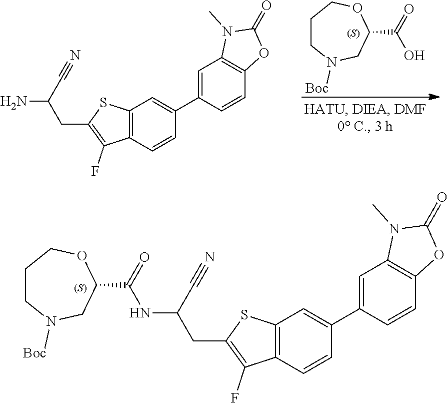

A solution of 2-amino-3-[3-fluoro-6-(3-methyl-2-oxo-1,3-benzoxazol-5-yl)-1-benzothiophen-2-yl]propanenitrile (89 mg, 0.24 mmol, 1.2 equiv) in DMF (2 mL) was treated with (2S)-4-(tert-butoxycarbonyl)-1,4-oxazepane-2-carboxylic acid (50 mg, 0.20 mmol, 1.0 equiv), DIEA (79 mg, 0.61 mmol, 3.0 equiv) followed by the addition of HATU (93 mg, 0.24 mmol, 1.2 equiv) in portions at 0° C. The resulting mixture was stirred for additional 2 h at 0° C. The residue was purified by silica gel column chromatography, eluted with PE/THF (1:1) to afford tert-butyl (2S)-2-({1-cyano-2-[3-fluoro-6-(3-methyl-2-oxo-1,3-benzoxazol-5-yl)-1-benzothiophen-2-yl]ethyl}carbamoyl)-1,4-oxazepane-4-carboxylate (100 mg, 82.4%) as colorless oil. LCMS (ES, m/z): [M+H]⁺: 595.

### Synthesis of (2S)—N-{1-cyano-2-[3-fluoro-6-(3-methyl-2-oxo-1,3-benzoxazol-5-yl)-1-benzothiophen-2-yl]ethyl}-1,4-oxazepane-2-carboxamide

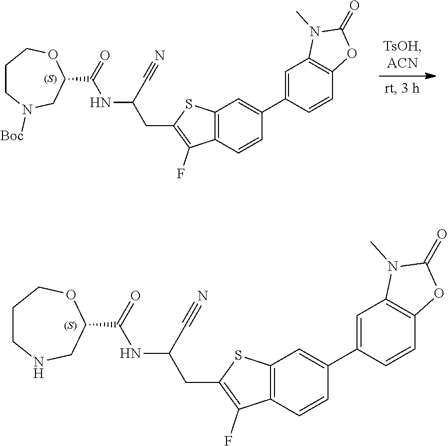

Into a 25 mL round-bottom flask were added tert-butyl (2S)-2-({1-cyano-2-[3-fluoro-6-(3-methyl-2-oxo-1,3-benzoxazol-5-yl)-1-benzothiophen-2-yl]ethyl}carbamoyl)-1,4-oxazepane-4-carboxylate (100 mg, 0.16 mmol, 1.0 equiv) in ACN (3 mL) and TsOH·H₂O (96 mg, 0.50 mmol, 3.0 equiv) at room temperature. The resulting mixture was stirred for 3 h at room temperature. The reaction solution was purified by reversed-phase flash chromatography with the following conditions: column, C₁₈ silica gel-120 g; mobile phase, MeCN in Water (0.1% NH₃·H₂O), 10% to 50% gradient in 10 min; detector, UV 254 nm. This resulted in (2S)—N-{1-cyano-2-[3-fluoro-6-(3-methyl-2-oxo-1,3-benzoxazol-5-yl)-1-benzothiophen-2-yl]ethyl}-1,4-oxazepane-2-carboxamide (20.5 mg, 24.6%) as white solid.

### Analytical Data

LCMS (ES, m/z): [M+H]⁺: 495.4

¹H NMR (400 MHz, DMSO-d₆) δ 8.80 (dd, J=8.5, 1.3 Hz, 1H), 8.34 (s, 1H), 7.83 (t, J=1.6 Hz, 2H), 7.72 (d, J=1.9 Hz, 1H), 7.53 (dd, J=8.4, 1.9 Hz, 1H), 7.44 (d, J=8.3 Hz, 1H), 5.08 (dtd, J=11.4, 8.4, 6.9 Hz, 1H), 4.06-3.84 (m, 2H), 3.74 (ddt, J=12.2, 8.1, 4.3 Hz, 1H), 3.56 (ddd, J=14.6, 6.9, 2.3 Hz, 1H), 3.51-3.42 (m, 4H), 3.11 (ddd, J=35.1, 14.2, 3.6 Hz, 1H), 2.87-2.54 (m, 3H), 1.88-1.62 (m, 2H).

### Example 14. Synthesis of compound 131a: (2S)—N-{1-cyano-2-[2-(3-methyl-2-oxo-1,3-benzoxazol-5-yl)-1-benzothiophen-5-yl]ethyl}-1,4-oxazepane-2-carboxamide

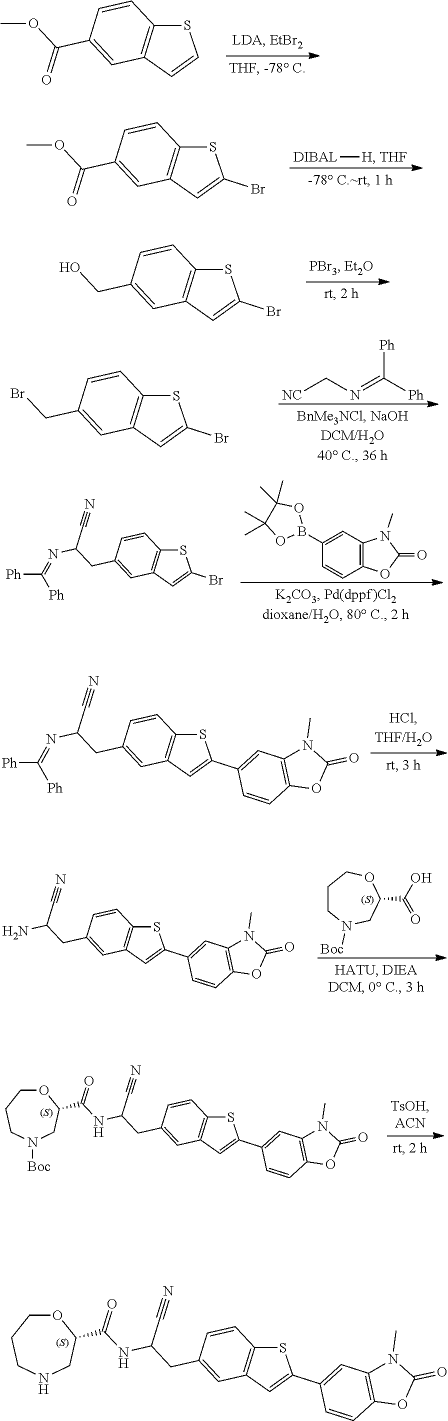

### Synthesis of methyl 2-bromobenzo[b]thiophene-5-carboxylate

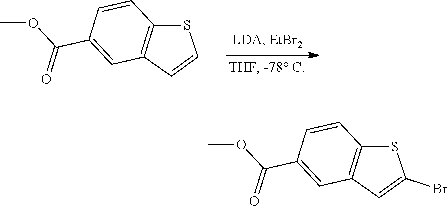

To a stirred solution of methyl benzo[b]thiophene-5-carboxylate (2 g, 10.40 mmol, 1.0 equiv) and THF (30 mL) was added LDA (2M in THF) (6.24 mL, 12.48 mmol, 1.2 equiv) dropwise at −78° C. under nitrogen atmosphere. The resulting mixture was stirred for 1 h at −78° C. under nitrogen atmosphere. To the above mixture was added dibromoethane (2.15 g, 11.44 mmol, 1.1 equiv) dropwise at −78° C. under nitrogen atmosphere. The resulting mixture was stirred for additional 1 h at room temperature under nitrogen atmosphere. The reaction was quenched by the addition of Water (50 mL) at room temperature, extracted with EtOAc (3×50 mL). The combined organic layer was dried over anhydrous Na₂SO₄. After filtration, the filtrate was concentrated under reduced pressure. The residue was purified by silica gel column chromatography, eluted with PE/EA (12:1) to afford methyl 2-bromobenzo[b]thiophene-5-carboxylate (1.7 g, 60%) as a light yellow oil. ¹H NMR (400 MHz, DMSO-d₆) δ 8.44 (s, 1H), 8.10 (d, J=8.5 Hz, 1H), 7.91 (dd, J=8.5, 1.6 Hz, 1H), 7.81 (s, 1H), 3.90 (s, 3H).

### Synthesis of (2-bromo-1-benzothiophen-5-yl)methanol

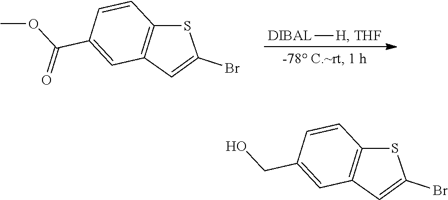

To a solution of methyl 2-bromobenzo[b]thiophene-5-carboxylate (1.3 g, 4.79 mmol, 1.0 equiv) and in tetrahydrofuran (25 mL) was added DIBAL-H (14.4 mL, 14.38 mmol, 3.0 equiv) (1 M in toluene) dropwise under nitrogen atmosphere at −78° C. After addition, the mixture was slowly warmed to room temperature, and stirred for 1 h. The reaction was quenched with HCl (1 N) (10 mL) at 0° C., diluted with water (20 mL), extracted with EtOAc (2×50 mL). The combined organic layer was washed with brine (50 mL), dried over anhydrous Na₂SO₄. After filtration, the filtrate was concentrated under reduced pressure. The residue was purified by silica gel column chromatography, eluted with PE/EA (3:1) to afford (2-bromo-1-benzothiophen-5-yl)methanol (1.0 g, 85.7%) as white solid. LCMS (ES, m/z): [M−H₂O+H]⁺: 225.

### Synthesis of 2-bromo-5-(bromomethyl)-1-benzothiophene

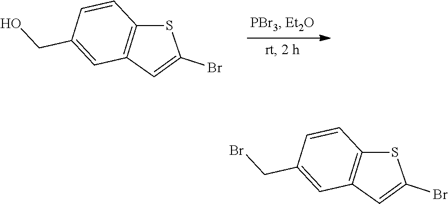 [SMILES: OCc1ccc2sc(Br)cc2c1 ; BrCc1ccc2sc(Br)cc2c1]

A solution of (2-bromo-1-benzothiophen-5-yl)methanol (1 g, 4.11 mmol, 1.0 equiv) in Et₂₀ (10 mL) was treated with PBr₃ (0.6 g, 2.05 mmol, 0.5 equiv) at 0° C. The resulting mixture was stirred for 2 h at room temperature. The reaction was quenched by water (20 mL) at 0° C., extracted with EtOAc (30 mL), washed with brine (30 mL), dried over anhydrous Na₂SO₄. After filtration, the filtrate was concentrated under reduced pressure. The residue was purified by silica gel column chromatography, eluted with PE/EA (3:1) to afford 2-bromo-5-(bromomethyl)-1-benzothiophene (700 mg, 55.6%) as white solid (no LCMS signal).

### Synthesis of 3-(2-bromo-1-benzothiophen-5-yl)-2-[(diphenylmethylidene)amino]propanenitrile

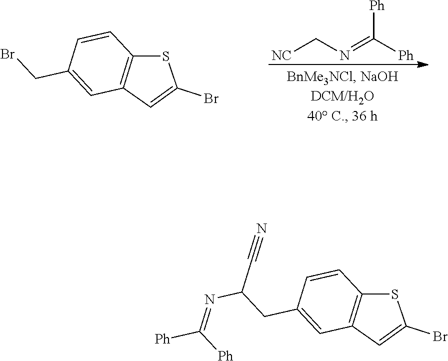 [SMILES: BrCc1ccc2sc(Br)cc2c1 ; N#CCN=C(c1ccccc1)c1ccccc1 ; N#CC(Cc1ccc2sc(Br)cc2c1)N=C(c1ccccc1)c1ccccc1]

To a solution of 2-bromo-5-(bromomethyl)-1-benzothiophene (600 mg, 1.96 mmol, 1.0 equiv) in DCM (5 mL) was added 2-[(diphenylmethylidene)amino]acetonitrile (432 mg, 1.96 mmol, 1.0 equiv), benzyltrimethylazanium chloride (36 mg, 0.19 mmol, 0.1 equiv), NaOH (159 mg, 3.92 mmol, 2.0 equiv), H₂O (1 mL) in sequence. The mixture was stirred for 36 h at 40° C. The reaction solution was diluted with water (20 mL), extracted with CH₂Cl₂ (2×30 mL). The combined organic layer was washed with brine (30 mL), dried over anhydrous Na₂SO₄. After filtration, the filtrate was concentrated under reduced pressure. The residue was purified by silica gel column chromatography, eluted with PE/THF (5:1) to afford 3-(2-bromo-1-benzothiophen-5-yl)-2-[(diphenylmethylidene)amino]propanenitrile (800 mg, crude) as yellow solid. LCMS (ES, m/z): [M+H]⁺: 445.

### Synthesis of 2-[(diphenylmethylidene)amino]-3-[2-(3-methyl-2-oxo-1,3-benzoxazol-5-yl)-1-benzothiophen-5-yl]propanenitrile

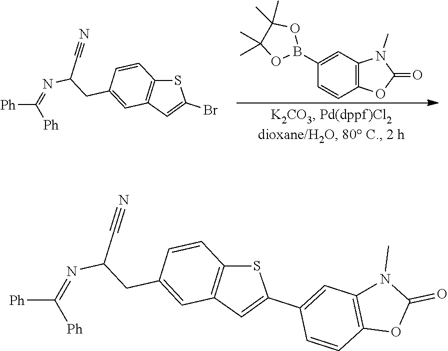 [SMILES: N#CC(Cc1ccc2sc(Br)cc2c1)N=C(c1ccccc1)c1ccccc1 ; Cn1c(=O)oc2ccc(B3OC(C)(C)C(C)(C)O3)cc21 ; Cn1c(=O)oc2ccc(-c3cc4cc(CC(C#N)N=C(c5ccccc5)c5ccccc5)ccc4s3)cc21]

To a solution of 3-(2-bromo-1-benzothiophen-5-yl)-2-[(diphenylmethylidene)amino]propanenitrile (350 mg, 0.78 mmol, 1.0 equiv), 3-methyl-5-(4,4,5-trimethyl-1,3,2-dioxaborolan-2-yl)-1,3-benzoxazol-2-one (205 mg, 0.78 mmol, 1.0 equiv) in 1,4-dioxane (10 mL) and H₂O (1 mL), were added K₂CO₃ (217 mg, 1.57 mmol, 2.0 equiv) and Pd(dppf)Cl₂ (58 mg, 0.07 mmol, 0.1 equiv) under nitrogen atmosphere. The mixture was stirred for 2 h at 80° C. The reaction was cooled to room temperature, concentrated to remove the solvent, the residue was purified by silica gel column chromatography, eluted with PE/THF (5:1) to afford 2-[(diphenylmethylidene)amino]-3-[2-(3-methyl-2-oxo-1,3-benzoxazol-5-yl)-1-benzothiophen-5-yl]propanenitrile (120 mg, 30%) as white solid. LCMS (ES, m/z): [M+H]⁺: 514.

### Synthesis of 2-amino-3-[2-(3-methyl-2-oxo-1,3-benzoxazol-5-yl)-1-benzothiophen-5-yl]propanenitrile

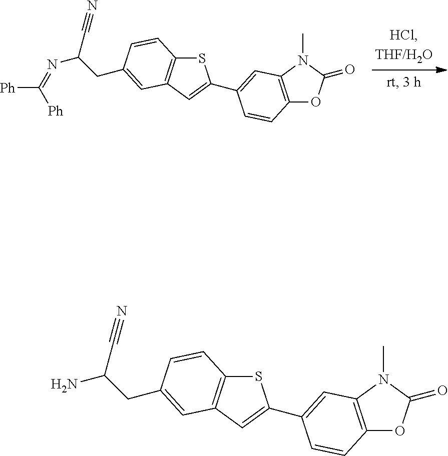 [SMILES: Cn1c(=O)oc2ccc(-c3cc4cc(CC(C#N)N=C(c5ccccc5)c5ccccc5)ccc4s3)cc21 ; Cn1c(=O)oc2ccc(-c3cc4cc(CC(N)C#N)ccc4s3)cc21]

Into a 100 mL round-bottom flask were added 2-[(diphenylmethylidene)amino]-3-[2-(3-meth yl-2-oxo-1,3-benzoxazol-5-yl)-1-benzothiophen-5-yl]propanenitrile (350 mg, 0.68 mmol, 1.0 equiv) and THF (30 mL), H₂O (3 mL) at room temperature. The resulting mixture was stirred for 3 h at room temperature. The mixture was basified to pH 12 with NaOH (2 N). The resulting mixture was extracted with EtOAc (2×50 mL). The combined organic layer was washed with brine (50 mL), dried over anhydrous Na₂SO₄. After filtration, the filtrate was concentrated under reduced pressure. The residue was purified by silica gel column chromatography, eluted with PE/THF (1:2) to afford 2-amino-3-[2-(3-methyl-2-oxo-1,3-benzoxazol-5-yl)-1-benzothiophen-5-yl]propanenitrile (120 mg, 50.4%) as white solid. LCMS (ES, m/z): [M+H]⁺: 349.

### Synthesis of tert-butyl (2S)-2-({1-cyano-2-[2-(3-methyl-2-oxo-1,3-benzoxazol-5-yl)-1-benzothiophen-5-yl]ethyl}carbamoyl)-1,4-oxazepane-4-carboxylate

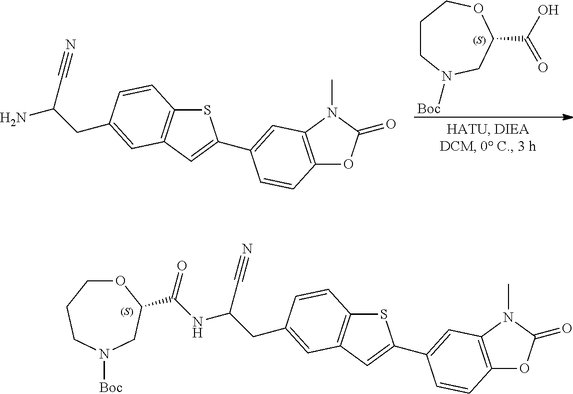

To a stirred mixture of 2-amino-3-[2-(3-methyl-2-oxo-1,3-benzoxazol-5-yl)-1-benzothiophen-5-yl]propanenitrile (110 mg, 0.31 mmol, 1.2 equiv), (2S)-4-(tert-butoxycarbonyl)-1,4-oxazepane-2-carboxylic acid (64 mg, 0.26 mmol, 1.0 equiv) and DIEA (102 mg, 0.78 mmol, 3.0 equiv) in DCM (5 mL) was added HATU (119 mg, 0.31 mmol, 1.2 equiv) in portions at 0° C. The resulting mixture was stirred for additional 3 h at 0° C. Concentrated under reduced pressure, the residue was purified by silica gel column chromatography, eluted with PE/THF (1:1) to afford tert-butyl (2S)-2-({1-cyano-2-[2-(3-methyl-2-oxo-1,3-benzoxazol-5-yl)-1-benzothiophen-5-yl]ethyl}carbamoyl)-1,4-oxazepane-4-carboxylate (120 mg, 79%) as white solid. LCMS (ES, m/z): [M+H]⁺: 577.

### Synthesis of (2S)—N-{1-cyano-2-[2-(3-methyl-2-oxo-1,3-benzoxazol-5-yl)-1-benzothiophen-5-yl]ethyl}-1,4-oxazepane-2-carboxamide

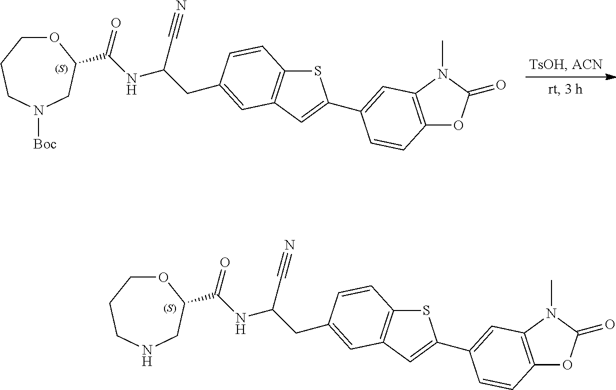

Into a 25 mL round-bottom flask were added tert-butyl (2S)-2-{[(1S)-1-cyano-2-[2-(3-methyl-2-oxo-1,3-benzoxazol-5-yl)-1-benzothiophen-5-yl]ethyl]carbamoyl}-1,4-oxazepane-4-carboxylate (120 mg, 0.20 mmol, 1.0 equiv), TsOH (108 mg, 0.62 mmol, 3.0 equiv) and ACN (3 mL) at room temperature. The resulting mixture was stirred for 3 h at room temperature. The reaction solution was purified by reversed-phase flash chromatography with the following conditions: column, C₁₈ silica gel-120 g; mobile phase, MeCN in Water (0.1% NH₃·H₂O), 10% to 50% gradient in 10 min; detector, UV 254 nm. This resulted in (2S)—N-{1-cyano-2-[2-(3-methyl-2-oxo-1,3-benzoxazol-5-yl)-1-benzothiophen-5-yl]ethyl}-1,4-oxazepane-2-carboxamide (16.3 mg, 16.44%) as white solid.

### Analytical Data

LCMS (ES, m/z): [M+H]⁺: 477.2

¹H NMR (400 MHz, DMSO-d₆) δ 8.61 (dd, J=8.4, 6.0 Hz, 1H), 7.93 (dd, J=8.2, 3.0 Hz, 1H), 7.86 (d, J=2.2 Hz, 1H), 7.75-7.73 (m, 2H), 7.51 (d, J=8.4 Hz, 1H), 7.43 (d, J=8.3 Hz 1H), 7.30 (d, J=8.3 Hz, 1H), 5.10-4.97 (m, 1H), 4.00-3.80 (m, 2H), 3.75-3.67 (m, 1H) 3.43 (s, 3H), 3.29-3.26 (m, 2H), 3.14-2.97 (m, 1H), 2.83-2.63 (m, 2H), 2.56-2.41 (m, 2H), 1.78-1.68 (m, 2H).

### Example 15. Synthesis of (2S)—N-(1-cyano-2-(3-(3-methyl-2-oxo-2,3-dihydrobenzo[d]oxazol-5-yl)bicyclo[1.1.1]pentan-1-yl)ethyl)-1,4-oxazepane-2-carboxamide (Compound 101e)

Methyl 3-phenylbicyclo[1.1.1]pentane-1-carboxylate,

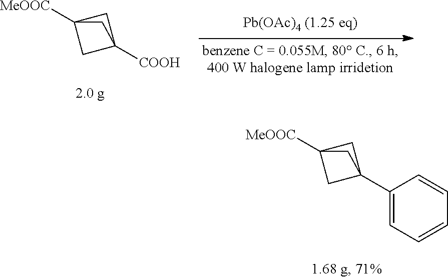 [SMILES: COC(=O)C12CC(C(=O)O)(C1)C2 ; COC(=O)C12CC(c3ccccc3)(C1)C2]

3-(methoxycarbonyl)bicyclo[1.1.1]pentane-1-carboxylic acid (2.00 g, 1 Eq, 11.8 mmol) and lead tetraacetate (6.86 g, 95% Wt, 1.25 Eq, 14.7 mmol) were dissolved in benzene (214 mL), flask was equipped in condenser closed with septum and small N₂ balloon (CO₂ releasing). The mixture was irradiated by a 400 W tungsten lamp over 6 h, large quantity of white inorganic precipitate was formed.

After cooling down the reaction flask, white suspension was filtered through short pad (30 g) of celite and a celite pad was washed with a 1:4 ethyl acetate: cyclohexane mix (3×100 mL). The clear colourless filtrate was thereafter concentrated under reduced pressure to give 2.82 g of crude.

Purification by flash column chromatography—80 g [cHex/EA]—0%-1CV, next 0->15%-2CV, and 15%-4CV. To afford methyl 3-phenylbicyclo[1.1.1]pentane-1-carboxylate as colourless oil (1.68 g, 8.31 mmol—71% yield).
TLC: cHex/DCM/EA 90:5:5,
Product can be visualised by KMnO₄ stain.

1H NMR (400 MHz, DMSO) δ 7.35-7.29 (m, 2H), 7.28-7.21 (m, 3H), 3.64 (s, 3H), 2.26 (s, 6H).

### 3-Phenylbicyclo[1.1.1]pentane-1-carboxylic acid

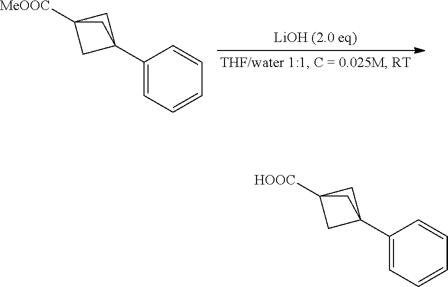 [SMILES: COC(=O)C12CC(c3ccccc3)(C1)C2 ; O=C(O)C12CC(c3ccccc3)(C1)C2]

To a biphasic solution of methyl 3-phenylbicyclo[1.1.1]pentane-1-carboxylate (1600 mg, 1 Eq, 7.911 mmol) in a mixture of THE (79.0 mL) and H₂O (79.0 mL), lithium hydroxide monohydrate (663.9 mg, 2 Eq, 15.82 mmol) was added in one portion. Reaction mixture was stirred at 25° C. for 16 hours. Conversion was determined by LCMS—full conversion. Basic mixture was diluted with 200 mL of water and washed with DCM 2×50 mL. LCMS of organic phase—confirms no product, only impurities. Water phase was acidified with 1M KHSO₄ (20 mL) and extracted with EtOAc (3×100 mL). Combined organics were dried over anhydrous Na₂SO₄, filtered and concentrated to dryness to afford 3-phenylbicyclo[1.1.1]pentane-1-carboxylic acid as white solid (1.31 g, 6.96 mmol—88% yield).

Alternatively:

To a biphasic solution of crude methyl 3-phenylbicyclo[1.1.1]pentane-1-carboxylate (assumed 4750 mg, 1 Eq, 23.50 mmol) in a mixture of THE (235 mL) and H₂O (235 mL), excess of sodium hydroxide (2.82 g, 3 Eq, 70.50 mmol) was added in one portion. Reaction mixture was stirred at 25° C. for 16 hours. Conversion was determined by LCMS—full conversion. Purification as described above, this method leads to 3-phenylbicyclo[1.1.1]pentane-1-carboxylic acid in little bit better total yield (2.96 g, 15.70 mmol—67% yield over two steps).

Product can be visualised by KMnO₄ stain.

1H NMR (400 MHz, DMSO) δ 12.40 (s, 1H), 7.35-7.29 (m, 2H), 7.27-7.20 (m, 3H), 2.21 (s, 6H).

### 3-Phenylbicyclo[1.1.1]pentane-1-carboxamide

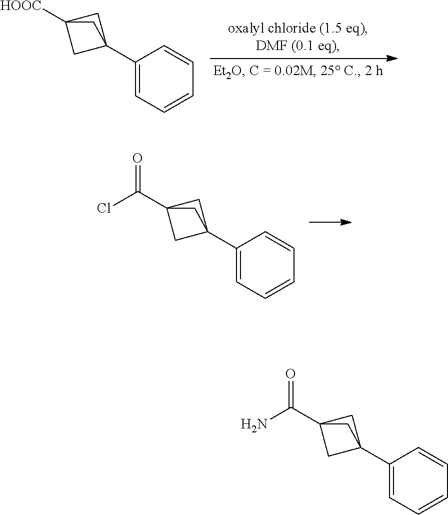 [SMILES: O=C(O)C12CC(c3ccccc3)(C1)C2 ; O=C(Cl)C12CC(c3ccccc3)(C1)C2 ; NC(=O)C12CC(c3ccccc3)(C1)C2]

To a solution of 3-(methoxycarbonyl)bicyclo[1.1.1]pentane-1-carboxylic acid (1430 mg, 1 Eq, 7.597 mmol) and oxalyl chloride (1253 mg, 1.3 Eq, 9.876 mmol, 864.5 μL) in Et₂O (38 mL), DMF (55 mg, 0.1 Eq, 0.76 mmol, 60 L) as the catalyst was added dropwise. The mixture was stirred around 2 h, until no more gas evolution was observed.

TLC—[EA]->[20 μL sample was taken and quenched with MeOH], visualization by KMnO₄ stain, shows full consumption of SM—formation of less polar spot.

The reaction mixture was concentrated to dryness on rotary evaporator to obtain orange liquid, used crude in next step.

Crude 3-phenylbicyclo[1.1.1]pentane-1-carbonyl chloride (assumed 1.570 g, 1 Eq, 7.597 mmol) was dissolved in DCM (44 mL) and added dropwise to the cold (−20° C.) solution of ammonia in MeOH (5.175 g, 43.5 mL, 7 molar, 40 Eq, 304 mmol) over 15 minutes. The mixture was stirred at −20° C. additional 2 h and allowed to reach 25° C. overnight (16 h).

LCMS shows full conversion of starting material.

The reaction mixture was concentrated under vacuum, and co-evaporated with CH₂Cl₂ twice. The solid was dissolved in DCM (150 mL) and washed with NaHCO₃/brine [2:8] (150 mL) and brine (150 mL), dried and evaporated to obtain 3-phenylbicyclo[1.1.1]pentane-1-carboxamide (1.35 g, 7.21 mmol—95% yield) as white solid.

1H NMR (400 MHz, DMSO) δ 7.36-7.27 (m, 3H), 7.27-7.18 (m, 3H), 6.96 (s, 1H), 2.14 (s, 6H).

Alternatively:

Crude 3-phenylbicyclo[1.1.1]pentane-1-carbonyl chloride (assumed 1.437 g, 1 Eq, 6.954 mmol) was dissolved in DCM (70 mL) and cooled down to 0° C., ammonium chloride (1.488 g, 4 Eq, 27.82 mmol) was added in one portion followed by dropwise addition of triethylamine (7.037 g, 9.69 mL, 10 Eq, 69.54 mmol). The mixture was stirred for 2 h in 0° C. and allowed to reach 25° C. overnight, stirring was continued over weekend. LCMS shows a mix of acid and amide. Compounds were separated by 2M HCl/1M NaOH extractions . . . .

### 3-Phenylbicyclo[1.1.1]pentane-1-carbonitrile

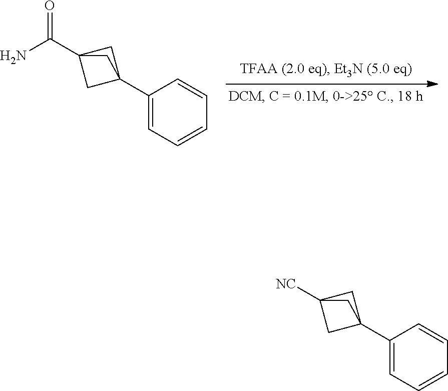 [SMILES: NC(=O)C12CC(c3ccccc3)(C1)C2 ; N#CC12CC(c3ccccc3)(C1)C2]

To an ice-cold solution of 3-phenylbicyclo[1.1.1]pentane-1-carboxamide (1.311 g, 1 Eq, 7.000 mmol) in dry DCM (70.00 mL), triethylamine (3.542 g, 4.88 mL, 5 Eq, 35.00 mmol) was added followed by dropwise addition of trifluoroaceticanhydride (2.940 g, 1.955 mL, 2 Eq, 14.00 mmol). The resulting reaction mixture was allowed to reach 25° C. and stirring was continued over 18 h. After LCMS shows full conversion of starting material flask was cooled by ice bath, and reaction was quenched with NaHCO₃ (30 mL), extracted with DCM (2×50 mL), next the organic layer was washed with 1N KHSO₄ to pH 2, water and brine, organic extract was dried over anhydrous Na₂SO₄.

Purification by flash column chromatography—20 g [pentane/Et₂O]—0%-1CV, next 0->15%-1CV, and 15%-4CV. To afford 3-phenylbicyclo[1.1.1]pentane-1-carbonitrile as colourless oil (1.185 g, 7.00 mmol->99% yield).

1H NMR (400 MHz, CDCl3) δ 7.35-7.27 (m, 3H), 7.19-7.15 (m, 2H), 2.51 (s, 6H).

### 5-(4-(3-cyanobicyclo[1.1.1]pentan-1-yl)phenyl)-5H-thianthren-5-ium tetrafluoroborate

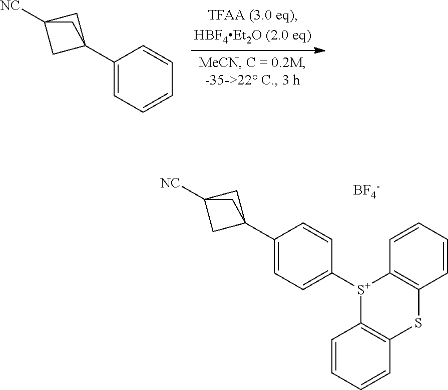 [SMILES: N#CC12CC(c3ccccc3)(C1)C2 ; N#CC12CC(c3ccc([S+]4c5ccccc5Sc5ccccc54)cc3)(C1)C2 ; "BF4-"]

Under an ambient atmosphere, a 50 mL RBF was charged sequentially with 3-phenylbicyclo[1.1.1]pentane-1-carbonitrile (694 mg, 4.10 mmol), thianthrene-S-oxide (1.0 g, 1.05 Eq, 4.30 mmol), and dry MeCN (20.5 ml, c=0.20 M). After most of the solids had dissolved (ca. 5 min), the solution was cooled to −35° C., next TFAA (2.58 g, 1.74 mL, 3.0 Eq, 12.30 mmol) was added in one portion, followed by dropwise addition of tetrafluoroboric acid (1.32 g, 1.11 mL, 2.0 Eq, 8.20 mmol). The mixture was allowed to warm to 23° C. over a period of 1.5 h and stirred in rt for next 1.5 h. LCMS—shows full conversion of SM. Then, the solution was diluted with 150 ml DCM and poured into a separatory funnel. The organic layer was washed with 50 ml of NaHCO₃/100 mL brine and water phase was extracted three times with 50 ml DCM. Combined organic extracts were washed with 150 ml of 10% water solution of NaBF₄ and dried with Na₂SO₄, filtered, and concentrated under reduced pressure. The crude was purified by column: FCC-12 g, silica, [DCM/MeOH—2CV-100%—DCM, then 4CV 0->10%, and 4CV-10% (Pure product at 8-10% methanol). To obtain product (1.35 g, 2.86 mmol, 70%).

TLC (DCM:MeOH 9:1), Product rf 0.42.

1H NMR (400 MHz, DMSO) δ 8.56 (d, J=6.8 Hz, 2H), 8.05 (dd, J=7.8, 1.2 Hz, 2H), 7.92 (td, J=7.7, 1.5 Hz, 2H), 7.85 (td, J=7.7, 1.4 Hz, 2H), 7.38 (d, J=8.7 Hz, 2H), 7.16 (d, J=8.6 Hz, 2H), 2.50 (s, 6H).

### 5-(4-(3-(methoxycarbonyl)bicyclo[1.1.1]pentan-1-yl)phenyl)-5H-thianthren-5-ium tetrafluoroborate

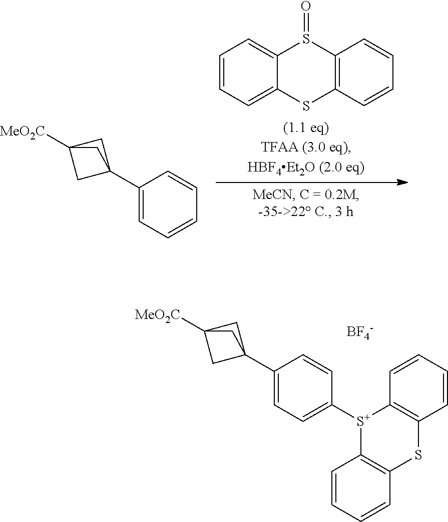 [SMILES: O=S1c2ccccc2Sc2ccccc21 ; COC(=O)C12CC(c3ccccc3)(C1)C2 ; COC(=O)C12CC(c3ccc([S+]4c5ccccc5Sc5ccccc54)cc3)(C1)C2 ; "BF4-"]

Under an ambient atmosphere, a 50 mL RBF was charged sequentially with 3-phenylbicyclo[1.1.1]pentane-1-carbonitrile (1.75 g, 8.65 mmol), thianthrene-S-oxide (2.21 g, 1.10 Eq, 9.52 mmol), and dry MeCN (43.2 ml, c=0.20 M). After most of the solids had dissolved (ca. 5 min), the solution was cooled to −35° C., next TFAA (5.45 g, 3.66 mL, 3.0 Eq, 25.96 mmol) was added in one portion, followed by dropwise addition of tetrafluoroboric acid (2.78 g, 2.35 mL, 2.0 Eq, 17.31 mmol). The mixture was allowed to warm to 23° C. over a period of 1.5 h and stirred in rt for the next 1.5 h. LCMS—shows full conversion of SM. Then, the solution was diluted with 150 ml DCM and poured into a separatory funnel. The organic layer was washed with 50 ml of NaHCO₃/100 mL brine and water phase was extracted three times with 50 ml DCM. Combined organic extracts were washed with 150 ml of 10% water solution of NaBF₄ and dried with Na₂SO₄, filtered, and concentrated under reduced pressure. The crude was purified by column: FCC-12 g, silica, [DCM/MeOH—2CV-100%—DCM, then 4CV 0->10%, and 4CV-10% (Pure product at 8-10% methanol). To obtain product (3.06 g, 6.08 mmol, 70%).

TLC (DCM:MeOH 9:1), Product rf 0.42.

1H NMR (400 MHz, DMSO) δ 8.56 (dd, J=7.9, 1.3 Hz, 2H), 8.06 (dd, J=7.9, 1.2 Hz, 2H), 7.92 (td, J=7.8, 1.5 Hz, 2H), 7.85 (td, J=7.7, 1.4 Hz, 2H), 7.41 (d, J=8.7 Hz, 2H), 7.16 (d, J=8.6 Hz, 2H), 3.61 (s, 3H), 2.24 (s, 6H).

### methyl 3-(4-hydroxyphenyl)bicyclo[1.1.1]pentane-1-carboxylate

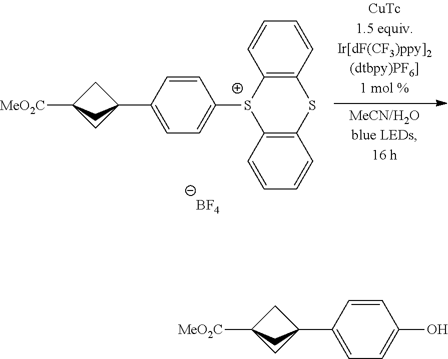

A vial charged with 5-(4-(3-(methoxycarbonyl)bicyclo[1.1.1]pentan-1-yl)phenyl)-5H-thianthren-5-ium tetrafluoroborate (252 mg, 1 Eq, 0.500 mmol), Copper(I) thiophene-2-carboxylate (143 mg, 1.5 Eq, 750 mol) and (4,4′-Di-t-butyl-2,2′-bipyridine)bis[3,5-difluoro-2-(5-trifluoromethyl-2-pyridinyl-kN)phenyl-kC]iridium(III) hexafluorophosphate (5.61 mg, 0.01 Eq, 5.00 mol). Vial was evacuated and backfilled with nitrogen three times. MeCN (2.50 mL) and H₂O (180 mg, 180 μL, 20 Eq, 10.0 mmol) were added. Blue LED lamp (450 nm) was installed. The reaction mixture was stirred overnight under irradiation. Lamp was switched off, addition of EtOAc, filtration over SiO₂, vial concentration to dryness. The crude was purified by MPLC Biotage Cyclohexane to EtOAc to afford methyl 3-(4-hydroxyphenyl)bicyclo[1.1.1]pentane-1-carboxylate (76.0 mg, 70%).

¹H NMR (400 MHz, CDCl₃) δ 7.12-7.06 (m, 2H), 6.80-6.75 (m, 2H), 3.71 (s, 3H), 2.28 (s, 6H).

### Ethyl/methyl 3-(4-hydroxy-3-nitrophenyl)bicyclo[1.1.1]pentane-1-carboxylate

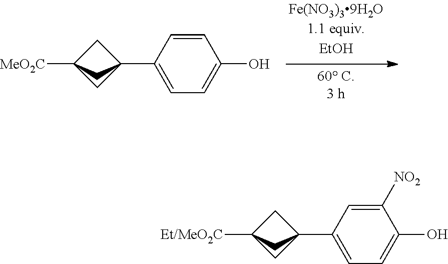

To a solution of methyl 3-(4-hydroxyphenyl)bicyclo[1.1.1]pentane-1-carboxylate (305.6 mg, 1 Eq, 1.400 mmol) in EtOH (14.00 mL) was added in one portion iron(III) nitrate nonahydrate (622.2 mg, 1.1 Eq, 1.540 mmol). The mixture was stirred at 60° C. for 3 h. Aqueous 6N HCl was added to the brown solution and the mixture was extracted with DCM. The organic phases were combined, washed with brine, dried over MgSO₄ and concentrated under reduced pressure. The crude was purified by column chromatography on silica gel by using hexane/EtOAc as the eluent to afford methyl 3-(4-hydroxy-3-nitrophenyl)bicyclo[1.1.1]pentane-1-carboxylate (158 mg, 600 mol, 42.9%). Mixture of Me and Et esters.

¹H NMR (400 MHz, CDCl₃) δ 10.52 (s, 1H), 7.89 (d, J=2.2 Hz, 1H), 7.44 (dd, J=8.6, 2.2 Hz, 1H), 7.11 (d, J=8.6 Hz, 1H), 4.17 (q, J=7.1 Hz, 1H), 3.72 (s, 1H), 2.33 (2 s, 6H), 1.29 (t, J=7.1 Hz, 2H).

### Ethyl/methyl 3-(3-amino-4-hydroxyphenyl)bicyclo[1.1.1]pentane-1-carboxylate

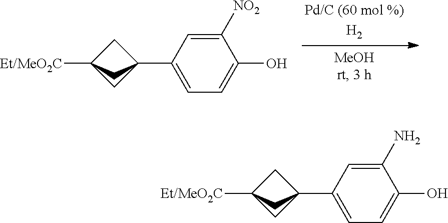

A flask was charged with methyl 3-(4-hydroxy-3-nitrophenyl)bicyclo[1.1.1]pentane-1-carboxylate (158 mg, 1 Eq, 600 mol), palladium on carbon (36.8 mg, 0.576 Eq, 346 mol) and MeOH (3.00 mL). Evacuated and backfilled with N₂ 3 times then with H₂ three times. The reaction mixture was filtered after 3 hours. Solvent was removed under vacuum. Crude NMR: pure. Assumed quant.

¹H NMR (60 MHz, CDCl₃) δ 6.59 (s, 3H), 4.17 (m, 2H), 3.71 (s, 1H), 2.23 (s, 6H), 1.28 (t, J=7.2 Hz, 2H).

### Ethyl/methyl 3-(2-oxo-2,3-dihydrobenzo[d]oxazol-5-yl)bicyclo[1.1.1]pentane-1-carboxylate

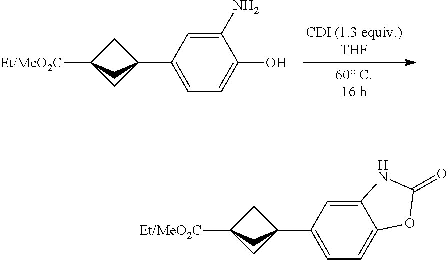

A 4-mL sealed tube was charged with ethyl/methyl 3-(3-amino-4-hydroxyphenyl)bicyclo[1.1.1]pentane-1-carboxylate (140.0 mg, 1 Eq, 600.2 mol)+di(1H-imidazol-1-yl)methanone (126.5 mg, 1.3 Eq, 780.2 mol) and THF (1.200 mL). The reaction mixture was stirred at 65° C. for 16 hours. It was then allowed to cool to rt, addition of water and extraction with EtOAc. The combined organic layers were dried over Na₂SO₄, filtered and concentrated. Assumed quant.

### Ethyl/methyl 3-(3-methyl-2-oxo-2,3-dihydrobenzo[d]oxazol-5-yl)bicyclo[1.1.1]pentane-1-carboxylate

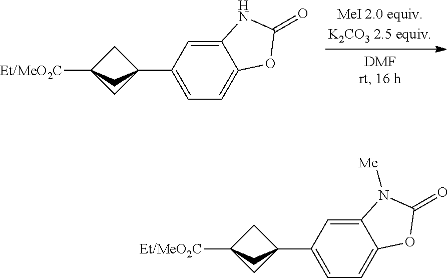

In a 50-mL RBF, to a solution of ethyl/methyl 3-(2-oxo-2,3-dihydrobenzo[d]oxazol-5-yl)bicyclo[1.1.1]pentane-1-carboxylate (155.61 mg, 1 Eq, 600.200 μmol) in DMF (2.00067 mL) was added at rt potassium carbonate (207.37 mg, 2.5 Eq, 1.50050 mmol) and methyl iodide (170.38 mg, 75.06 μL, 2 Eq, 1.20040 mmol). The reaction mixture was stirred at rt for 16 hours. Water and the precipitate was filtered off. The cake was dissolved in EtOAc and dried over anhydrous Na₂SO₄ and concentrated to dryness. The residue was purified by FC (SiO₂, cyclohexane to cyclohexane:EtOAc) to afford ethyl/methyl 3-(3-methyl-2-oxo-2,3-dihydrobenzo[d]oxazol-5-yl)bicyclo[1.1.1]pentane-1-carboxylate (145.7 mg, 533.1 μmol, 88.83%) as a solid. Mixture of Me and Et esters.

¹H NMR (60 MHZ, CDCl₃) δ 7.22-6.75 (m, 3H), 4.12 (m, 2H), 3.72 (s, 1H), 3.40 (s, 3H), 1.23 (m, 3H).

### 3-(3-methyl-2-oxo-2,3-dihydrobenzo[d]oxazol-5-yl)bicyclo[1.1.1]pentane-1-carboxylic acid

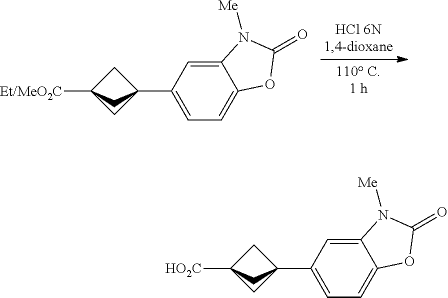

to a solution of methyl 3-(3-methyl-2-oxo-2,3-dihydrobenzo[d]oxazol-5-yl)bicyclo[1.1.1]pentane-1-carboxylate (146 mg, 1 Eq, 534 μmol) in 1,4-Dioxane (3.56 mL) was added at rt HCl 6N (1.78 mL). The rxn was stirred at 110° C. for 2 hours. Water was added and the precipitate was filtered off. The cake was dissolved in EtOAc and dried over anhydrous Na₂SO₄ and concentrated to dryness. Assumed quant.

¹H NMR (60 MHZ, CDCl₃) δ 8.77 (s, 1H), 7.21-6.75 (m, 3H), 3.40 (s, 3H), 2.37 (s, 6H).

### methyl 2-(bis(tert-butoxycarbonyl)amino)-3-(3-(3-methyl-2-oxo-2,3-dihydrobenzo[d]oxazol-5-yl)bicyclo[1.1.1]pentan-1-yl)propanoate

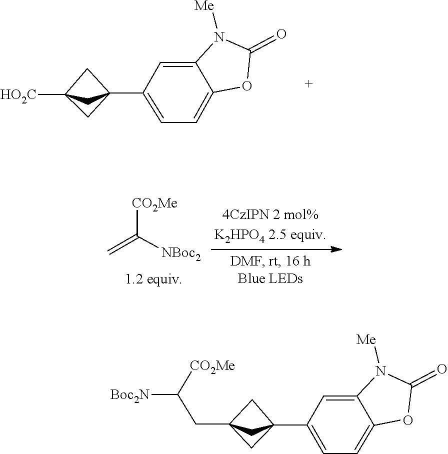

Vial charged with 3-(3-methyl-2-oxo-2,3-dihydrobenzo[d]oxazol-5-yl)bicyclo[1.1.1]pentane-1-carboxylic acid (50.000 mg, 1 Eq, 192.86 mol), 4CzIPN (2.8576 mg, 0.02 Eq, 3.8571 mol), methyl 2-(bis(tert-butoxycarbonyl)amino)acrylate (69.738 mg, 1.2 Eq, 231.43 mol), Potassium phosphate, dibasic (83.975 mg, 2.5 Eq, 482.14 mol) and DMF (1.9286 mL). Degassed for 15 min. Lamp 450 blue LEDS was installed and the reaction mixture was irradiated for 16 hours. Addition of water and extraction with EtOAc, dried over Na₂ₛO₄, concentrated to dryness. Purification by MPLC (cyclohexane/EtOAc) to afford methyl 2-(bis(tert-butoxycarbonyl)amino)-3-(3-(3-methyl-2-oxo-2,3-dihydrobenzo[d]oxazol-5-yl)bicyclo[1.1.1]pentan-1-yl)propanoate (47.2 mg, 91.4 mol, 47.4%).

¹H NMR (400 MHz, CDCl₃) δ 7.13-7.03 (m, 2H), 6.88 (d, J=1.9 Hz, 1H), 4.98 (dd, J=7.6, 3.5 Hz, 1H), 3.71 (s, 3H), 3.39 (s, 3H), 2.25-2.19 (m, 1H), 1.82 (t, J=7.8 Hz, 6H), 1.51 (s, 18H).

### 2-amino-3-(3-(3-methyl-2-oxo-2,3-dihydrobenzo[d]oxazol-5-yl)bicyclo[1.1.1]pentan-1-yl)propanoic acid hydrochloride

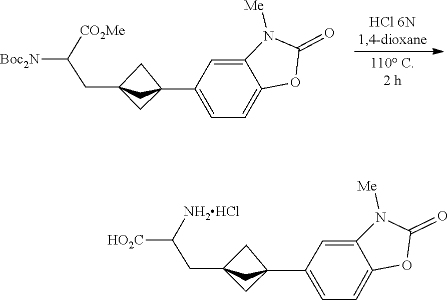

to a solution of methyl 2-(bis(tert-butoxycarbonyl)amino)-3-(3-(3-methyl-2-oxo-2,3-dihydrobenzo[d]oxazol-5-yl)bicyclo[1.1.1]pentan-1-yl)propanoate (47.2000 mg, 1 Eq, 91.368 mol) in 1,4-Dioxane (609.12 L) was added at rt HCl 6N (304.56 L). The reaction mixture was stirred at 110° C. for 2 hours. solvent was removed in vacuo. Assumed quant.

¹H NMR (400 MHz, D₂O) δ 6.89 (d, J=8.2 Hz, 1H), 6.84 (d, J=8.4 Hz, 1H), 6.78 (s, 1H), 4.12 (t, J=5.8 Hz, 1H), 3.66 (t, J=4.3 Hz, 1H), 3.12 (s, 3H), 2.34-2.21 (m, 2H), 2.02 (s, 6H).

### 2-((tert-butoxycarbonyl)amino)-3-(3-(3-methyl-2-oxo-2,3-dihydrobenzo[d]oxazol-5-yl)bicyclo[1.1.1]pentan-1-yl)propanoic acid

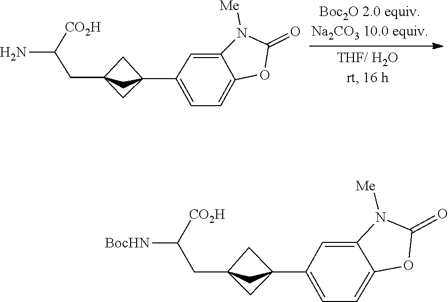 [SMILES: Cn1c(=O)oc2ccc([C@]34C[C@](CC(N)C(=O)O)(C3)C4)cc21 ; Cn1c(=O)oc2ccc([C@]34C[C@](CC(NC(=O)OC(C)(C)C)C(=O)O)(C3)C4)cc21]

A vial was charged with 2-amino-3-(3-(3-methyl-2-oxo-2,3-dihydrobenzo[d]oxazol-5-yl)bicyclo[1.1.1]pentan-1-yl)propanoic acid hydrochloride (30.955 mg, 1 Eq, 91.368 mol), sodium carbonate (96.841 mg, 10 Eq, 913.68 mol) and Di-tert-butyl dicarbonate (39.882 mg, 40.8 μL, 2.0 Eq, 182.74 mol) in a mixture of THF (913.68 L) and H₂O (913.68 μL). The reaction mixture was stirred at rt o/n. Addition of sat NaHCO₃, extraction with EtOAc, dried over Na₂SO₄ and concentrated to dryness to afford 2-((tert-butoxycarbonyl)amino)-3-(3-(3-methyl-2-oxo-2,3-dihydrobenzo[d]oxazol-5-yl)bicyclo[1.1.1]pentan-1-yl)propanoic acid (36.8 mg, 91.4 mol, 100%), assuming quantitative yield.

¹H NMR (400 MHz, CDCl₃) δ 7.07 (d, J=8.2 Hz, 1H), 6.90 (dd, J=8.2, 1.7 Hz, 1H), 6.75 (d, J=1.6 Hz, 1H), 5.08 (d, J=8.5 Hz, 1H), 4.41-4.32 (m, 1H), 3.37 (s, 3H), 2.04-1.88 (m, 8H), 1.45 (s, 9H).

### tert-butyl (1-amino-3-(3-(3-methyl-2-oxo-2,3-dihydrobenzo[d]oxazol-5-yl)bicyclo[1.1.1]pentan-1-yl)-1-oxopropan-2-yl)carbamate

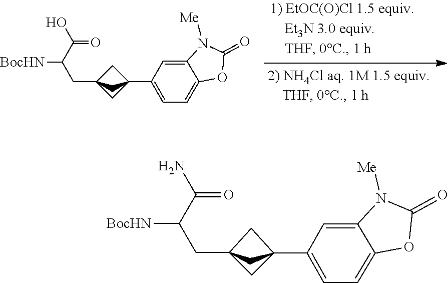 [SMILES: Cn1c(=O)oc2ccc([C@]34C[C@](CC(NC(=O)OC(C)(C)C)C(=O)O)(C3)C4)cc21 ; Cn1c(=O)oc2ccc([C@]34C[C@](CC(NC(=O)OC(C)(C)C)C(N)=O)(C3)C4)cc21]

To a solution of 2-((tert-butoxycarbonyl)amino)-3-(3-(3-methyl-2-oxo-2,3-dihydrobenzo[d]oxazol-5-yl)bicyclo[1.1.1]pentan-1-yl)propanoic acid (36.771 mg, 1 Eq, 91.368 mol) in dry THF (1.8274 mL), were added at 0° C. triethylamine (27.737 mg, 38.2 μL, 3.000 Eq, 274.10 mol) and Ethyl chloroformate (14.873 mg, 13.08 μL, 1.5 Eq, 137.05 mol). The reaction mixture was stirred at rt for 2 hours. The reaction is assumed quantitative. To it was added ammonia hydrochloride (7.331 mg, 137.05 μL, 1 molar, 1.5 Eq, 137.05 mol). The reaction mixture was stirred at rt for 3 hours. Addition of 1N KHSO₄, extraction with EtOAc three times, dried over Na₂SO₄, filtered and concentrated to dryness. Assumed quant.

### tert-butyl (1-cyano-2-(3-(3-methyl-2-oxo-2,3-dihydrobenzo[d]oxazol-5-yl)bicyclo[1.1.1]pentan-1-yl)ethyl)carbamate

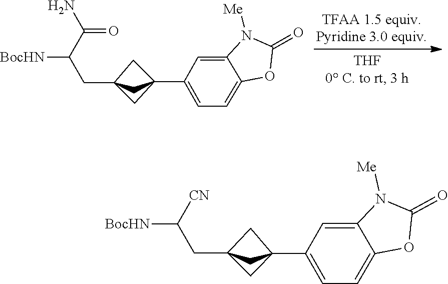 [SMILES: Cn1c(=O)oc2ccc([C@]34C[C@](CC(NC(=O)OC(C)(C)C)C(N)=O)(C3)C4)cc21 ; Cn1c(=O)oc2ccc([C@]34C[C@](CC(C#N)NC(=O)OC(C)(C)C)(C3)C4)cc21]

Trifluoroaceticanhydride (28.785 mg, 19.14 μL, 1.5 Eq, 137.05 mol) and pyridine (21.68 mg, 22.2 μL, 3 Eq, 274.10 mol) were added to an ice-cold solution of tert-butyl (1-amino-3-(3-(3-methyl-2-oxo-2,3-dihydrobenzo[d]oxazol-5-yl)bicyclo[1.1.1]pentan-1-yl)-1-oxopropan-2-yl)carbamate (36.681 mg, 1 Eq, 91.368 mol) in dry THE (913.68 L) at 0° C. The resulting reaction mixture was stirred for 2 h. The solvent was removed in vacuo, and the residue was dissolved in ethyl acetate. The organic layer was washed with 1N KHSO₄, water, and brine, dried over anhydrous Na₂SO₄, filtered and concentrated to dryness. Assumed quant. 1H NMR (400 MHz, CDCl₃) δ 7.12 (d, J=8.2 Hz, 1H), 6.96-6.92 (m, 1H), 6.79 (d, J=1.6 Hz, 1H), 5.00 (s, 1H), 4.62 (s, 1H), 3.40 (s, 3H), 2.14-2.03 (m, 8H).

### 2-amino-3-(3-(3-methyl-2-oxo-2,3-dihydrobenzo[d]oxazol-5-yl)bicyclo[1.1.1]pentan-1-yl)propanenitrile

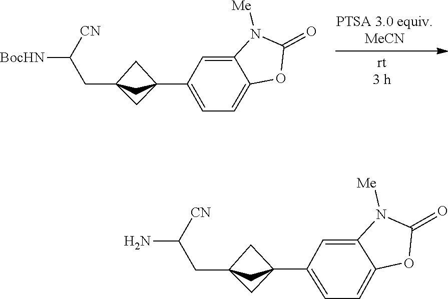 [SMILES: Cn1c(=O)oc2ccc([C@]34C[C@](CC(C#N)NC(=O)OC(C)(C)C)(C3)C4)cc21 ; Cn1c(=O)oc2ccc([C@]34C[C@](CC(N)C#N)(C3)C4)cc21]

Into a 10 mL-vial were added tert-butyl (1-cyano-2-(3-(3-methyl-2-oxo-2,3-dihydrobenzo[d]oxazol-5-yl)bicyclo[1.1.1]pentan-1-yl)ethyl)carbamate (35.035 mg, 1 Eq, 91.368 mol), MeCN (1 mL) and PTSOH (52.137 mg, 3 Eq, 274.10 mol), at room temperature. The resulting mixture was stirred for 3 h at room temperature. Reaction mixture was cooled by ice bath and basified to 8 with saturated NaHCO₃ (aq.), extracted with ethyl acetate (15 mL×3). The combined organic layer was washed with brine (100 mL), dried over anhydrous MgSO₄. After filtration, the filtrate was concentrated under reduced pressure. NMR: ok—assumed quant.

¹H NMR (400 MHz, CDCl₃) δ 7.10 (d, J=8.2 Hz, 1H), 6.93 (dd, J=8.2, 1.6 Hz, 1H), 6.77 (d, J=1.7 Hz, 1H), 3.73 (dd, J=8.1, 6.0 Hz, 1H), 3.39 (s, 3H), 2.09 (s, 6H), 2.05 (dd, J=8.8, 7.0 Hz, 2H).

### tert-butyl(2S)-2-((1-cyano-2-(3-(3-methyl-2-oxo-2,3-dihydrobenzo[d]oxazol-5-yl)bicyclo[1.1.1]pentan-1-yl)ethyl)carbamoyl)-1,4-oxazepane-4-carboxylate

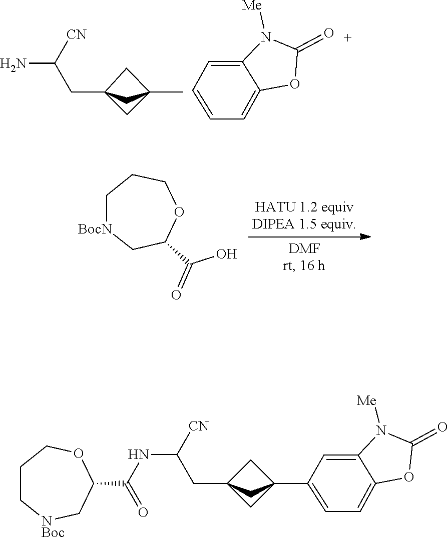

To a solution of 2-amino-3-(3-(3-methyl-2-oxo-2,3-dihydrobenzo[d]oxazol-5-yl)bicyclo[1.1.1]pentan-1-yl)propanenitrile (25.887 mg, 1 Eq, 91.368 mol) and (S)-4-(tert-butoxycarbonyl)-1,4-oxazepane-2-carboxylic acid (24.651 mg, 1.1 Eq, 100.50 mol) in dry DMF (1 mL) was added HATU (41.690 mg, 1.2 Eq, 109.64 mol) and Diisopropylethylamine (17.714 mg, 23.7 μL, 1.5 Eq, 137.05 mol). The reaction mixture was stirred at rt for 16 hour. The reaction mixture was concentrated to give residue which was added saturated aqueous solution of NaHCO₃ (30 mL) at 0° C., and extracted with ethyl acetate (10 mL×3). The combined organic layers were washed with brine (20 mL), dried over Na₂SO₄, filtered and concentrated under reduced pressure to give crude product. FC DCM/MeOH 0 to 10% afforded product. (32.2 mg, 69%).

¹H NMR (400 MHz, CDCl₃) δ 7.10 (d, J=8.2 Hz, 1H), 6.92 (dt, J=8.0, 1.4 Hz, 1H), 6.76 (q, J=1.8 Hz, 1H), 4.92 (s, 1H), 4.26-3.98 (m, 3H), 3.65 (s, 1H), 3.59-3.50 (m, 1H), 3.39 (s, 3H), 2.08 (s, 6H), 1.46 (2 s, 9H).

### (2S)—N-(1-cyano-2-(3-(3-methyl-2-oxo-2,3-dihydrobenzo[d]oxazol-5-yl)bicyclo[1.1.1]pentan-1-yl)ethyl)-1,4-oxazepane-2-carboxamide

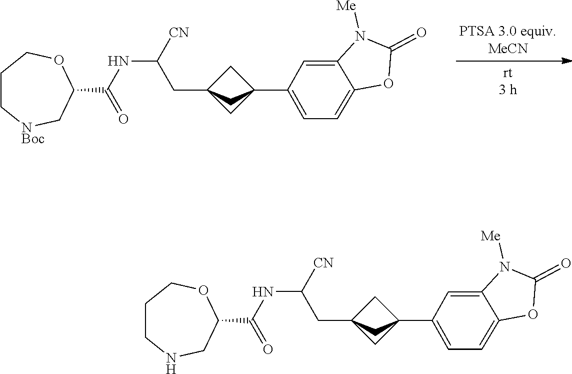 [SMILES: Cn1c(=O)oc2ccc([C@]34C[C@](CC(C#N)NC(=O)[C@@H]5CN(C(=O)OC(C)(C)C)CCCO5)(C3)C4)cc21 ; Cn1c(=O)oc2ccc([C@]34C[C@](CC(C#N)NC(=O)[C@@H]5CNCCCO5)(C3)C4)cc21]

A vial was charged with tert-butyl (2S)-2-((1-cyano-2-(3-(3-methyl-2-oxo-2,3-dihydrobenzo[d]oxazol-5-yl)bicyclo[1.1.1]pentan-1-yl)ethyl)carbamoyl)-1,4-oxazepane-4-carboxylate (32.2 mg, 1 Eq, 63.1 mol) and PTSOH (36.0 mg, 3 Eq, 189 mol). Addition of MeCN (1 mL). Stirring at rt for 3 hours. Addition of sat NaHCO₃ and extraction with EtOAc 3 times. Org layers dried over Na₂SO₄, concentrated to dryness. Purif by MPLC (DCM/MeOH) to afford (2S)—N-(1-cyano-2-(3-(3-methyl-2-oxo-2,3-dihydrobenzo[d]oxazol-5-yl)bicyclo[1.1.1]pentan-1-yl)ethyl)-1,4-oxazepane-2-carboxamide (13.3 mg, 32.4 mol, 51.4%).

¹H NMR (400 MHz, DMSO) δ 8.64 (t, J=8.1 Hz, 1H), 7.22 (d, J=8.1 Hz, 1H), 7.08 (dd, J=3.2, 1.6 Hz, 1H), 6.91 (dt, J=8.1, 1.9 Hz, 1H), 4.79-4.70 (m, 1H), 4.08 (ddd, J=17.1, 8.4, 3.5 Hz, 1H), 3.93 (ddt, J=12.3, 6.2, 4.4 Hz, 1H), 3.75 (dtd, J=12.3, 7.9, 4.2 Hz, 1H), 3.29 (s, 2H), 2.94-2.73 (m, 3H), 2.16-2.10 (m, 2H), 1.96 (d, J=4.2 Hz, 6H).

### Example 16. synthesis of (2S)—N-(1-cyano-2-(6-(3-methyl-2-oxo-2,3-dihydrobenzo[d]oxazol-5-yl)-1H-indol-2-yl)ethyl)-1,4-oxazepane-2-carboxamide (Compound 128b)

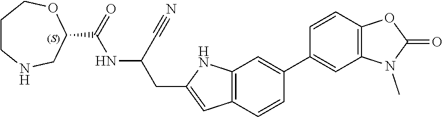

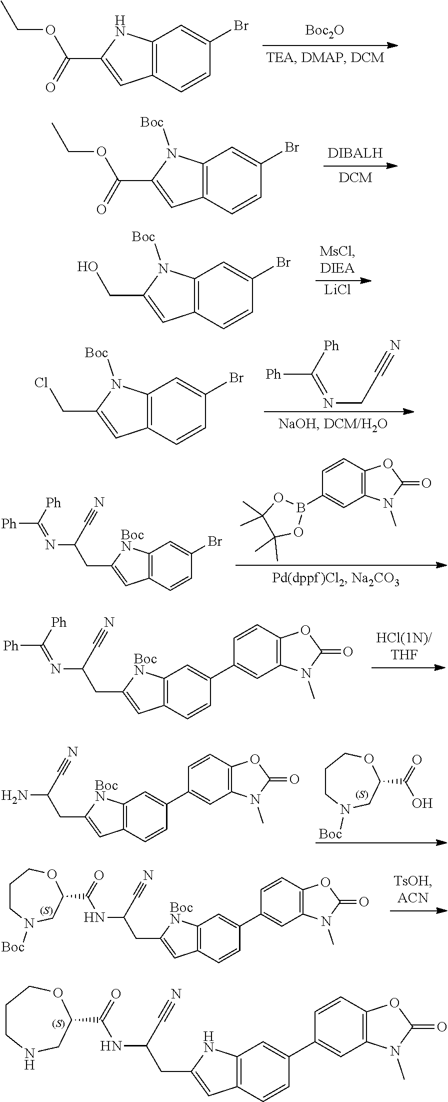

### Synthesis of 1-(tert-butyl) 2-ethyl 6-bromo-1H-indole-1,2-dicarboxylate

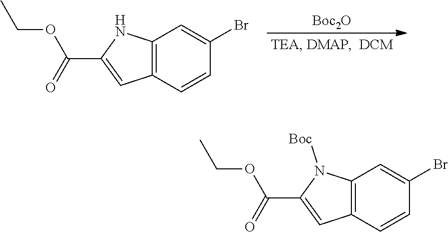 [SMILES: "Boc2O" ; CCOC(=O)c1cc2ccc(Br)cc2[nH]1 ; CCOC(=O)c1cc2ccc(Br)cc2n1C(=O)OC(C)(C)C]

A solution of ethyl 6-bromo-1H-indole-2-carboxylate (2 g, 7.46 mmol, 1.0 equiv) in DCM (20 mL) was treated with TEA (1.51 g, 14.92 mmol, 2.0 equiv) and DMAP (0.09 g, 0.75 mmol, 0.1 equiv) at room temperature followed by the addition of Boc₂O (2.44 g, 11.19 mmol, 1.5 equiv) in portions at 0° C. The resulting mixture was stirred for 2 h at room temperature. The resulting mixture was diluted with DCM (30 mL). The resulting mixture was washed with water (3×30 mL), dried over anhydrous Na₂SO₄. After filtration, the filtrate was concentrated under reduced pressure. The crude product 1-tert-butyl 2-ethyl 6-bromoindole-1,2-dicarboxylate (2.6 g, 94.65%) as a yellow oil was used in the next step directly without further purification. LCMS (ES, m/z): [M+H]⁺: 368.

### Synthesis of tert-butyl 6-bromo-2-(hydroxymethyl)-1H-indole-1-carboxylate

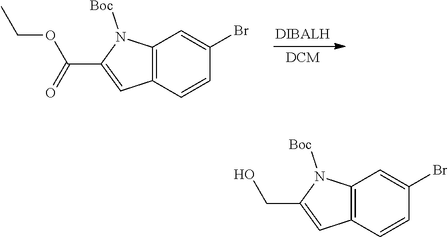 [SMILES: CCOC(=O)c1cc2ccc(Br)cc2n1C(=O)OC(C)(C)C ; CC(C)(C)OC(=O)n1c(CO)cc2ccc(Br)cc21]

A solution of 1-tert-butyl 2-ethyl 6-bromoindole-1,2-dicarboxylate (2.3 g, 6.25 mmol, 1.0 equiv) in DCM (35 mL) was treated with DIBAL-H (10.41 mL, 15.62 mmol, 2.5 equiv) dropwise at −78° C. The resulting mixture was stirred for 2 h at −40° C. The reaction was quenched by the addition of Water (20 mL) at 0° C. The resulting mixture was filtered, the filter cake was washed with EtOAc (2×50 mL). The filtrate was washed with 2×50 mL of water. The organic layer was dried over anhydrous Na₂SO₄. After filtration, the filtrate was concentrated under reduced pressure. The crude product tert-butyl 6-bromo-2-(hydroxymethyl)indole-1-carboxylate (1.8 g, 88.35%) as a yellow oil was used in the next step directly without further purification. LCMS (ES, m/z): [M+H]⁺: 326.

### Synthesis of tert-butyl 6-bromo-2-(chloromethyl)-1H-indole-1-carboxylate

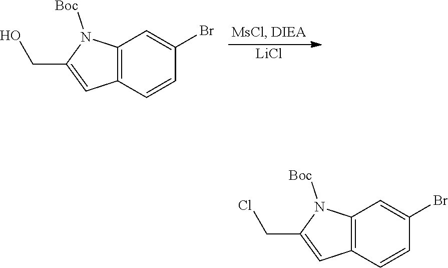

A solution of tert-butyl 6-bromo-2-(hydroxymethyl)indole-1-carboxylate (1.8 g, 5.52 mmol, 1.0 equiv) in DCM (60 mL) was treated with TEA (0.95 g, 9.38 mmol, 1.7 equiv) and LiCl (2.34 g, 55.18 mmol, 10.0 equiv) at room temperature followed by the addition of MsCl (1.07 g, 9.38 mmol, 1.7 equiv) dropwise at room temperature. The resulting mixture was stirred for 16 h at room temperature. The resulting mixture was washed with 2×30 mL of water. The organic layer was dried over anhydrous Na₂SO₄. After filtration, the filtrate was concentrated under reduced pressure. The crude product tert-butyl 6-bromo-2-(chloromethyl)indole-1-carboxylate (1.8 g, 94.65%) as a brown oil was used in the next step directly without further purification. LCMS (ES, m/z): [M+H]⁺: 344.

### Synthesis of tert-butyl 6-bromo-2-(2-cyano-2-((diphenylmethylene)amino)ethyl)-1H-indole-1-carboxylate

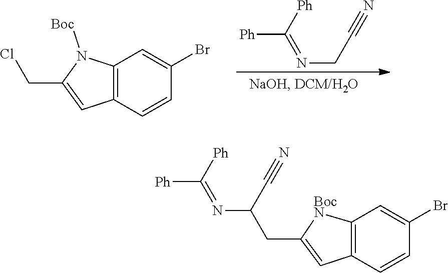 [SMILES: CC(C)(C)OC(=O)n1c(CCl)cc2ccc(Br)cc21 ; N#CCN=C(c1ccccc1)c1ccccc1 ; CC(C)(C)OC(=O)n1c(CC(C#N)N=C(c2ccccc2)c2ccccc2)cc2ccc(Br)cc21]

A mixture of tert-butyl 6-bromo-2-(chloromethyl)indole-1-carboxylate (1.8 g, 5.22 mmol, 1.0 equiv), NaOH (0.42 g, 10.45 mmol, 2.0 equiv) and 2-[(diphenylmethylidene)amino]acetonitrile (1.15 g, 5.22 mmol, 1.0 equiv), benzyl(chloro)trimethylamine (0.10 g, 0.52 mmol, 0.1 equiv) in DCM (20 mL) and H₂O (2 mL) was stirred for 8 h at 40° C. The mixture was allowed to cool down to room temperature. The resulting mixture was diluted with DCM (30 mL). The resulting mixture was washed with water (2×30 mL), dried over anhydrous Na₂SO₄. After filtration, the filtrate was concentrated under reduced pressure. The residue was purified by silica gel column chromatography, eluted with PE/EA (10:1) to afford tert-butyl 6-bromo-2-{2-cyano-2-[(diphenylmethylidene)amino]ethyl}-octahydroindole-1-carboxylate (1.2 g, 42.82%) as a brown oil. LCMS (ES, m/z): [M+H]⁺: 528.

### Synthesis of tert-butyl 2-(2-cyano-2-((diphenylmethylene)amino)ethyl)-6-(3-methyl-2-oxo-2,3-dihydrobenzo[d]oxazol-5-yl)-1H-indol-1-carboxylate

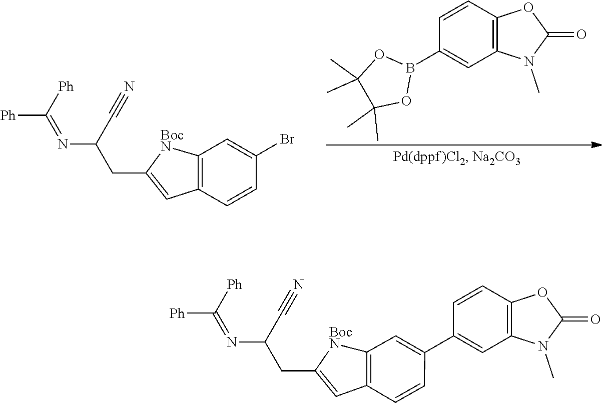 [SMILES: CC(C)(C)OC(=O)n1c(CC(C#N)N=C(c2ccccc2)c2ccccc2)cc2ccc(Br)cc21 ; Cn1c(=O)oc2ccc(B3OC(C)(C)C(C)(C)O3)cc21 ; Cn1c(=O)oc2ccc(-c3ccc4cc(CC(C#N)N=C(c5ccccc5)c5ccccc5)n(C(=O)OC(C)(C)C)c4c3)cc21]

A mixture of tert-butyl 6-bromo-2-{2-cyano-2-[(diphenylmethylidene)amino]ethyl}indole-1-carboxylate (1.2 g, 2.27 mmol, 1.0 equiv), 3-methyl-5-(4,4,5,5-tetramethyl-1,3,2-dioxaborolan-2-yl)-1,3-benzoxazol-2-one (0.62 g, 2.27 mmol, 1.0 equiv), Na₂CO₃ (0.48 g, 4.54 mmol, 2.0 equiv) and Pd(dppf)Cl₂ (0.17 g, 0.23 mmol, 0.1 equiv) in dioxane (12 mL) and H₂O (1.2 mL) was stirred for 16 h at 80° C. under nitrogen atmosphere. The mixture was allowed to cool down to room temperature. The resulting mixture was diluted with water (50 mL). The resulting mixture was extracted with EtOAc (3×50 mL). The combined organic layers were washed with water (2×50 mL), dried over anhydrous Na₂SO₄. After filtration, the filtrate was concentrated under reduced pressure. The residue was purified by silica gel column chromatography, eluted with PE/EA (5:1) to afford tert-butyl 2-{2-cyano-2-[(diphenylmethylidene)amino]ethyl}-6-(3-methyl-2-oxo-1,3-benzoxazol-5-yl)indole-1-carboxylate (1.2 g, 88.56%) as a brown solid. LCMS (ES, m/z): [M+H]⁺: 597.

### Synthesis of tert-butyl 2-(2-amino-2-cyanoethyl)-6-(3-methyl-2-oxo-2,3-dihydrobenzo[d]oxazol-5-yl)-1H-indole-1-carboxylate

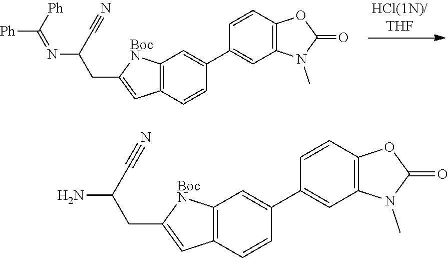 [SMILES: Cn1c(=O)oc2ccc(-c3ccc4cc(CC(C#N)N=C(c5ccccc5)c5ccccc5)n(C(=O)OC(C)(C)C)c4c3)cc21 ; Cn1c(=O)oc2ccc(-c3ccc4cc(CC(N)C#N)n(C(=O)OC(C)(C)C)c4c3)cc21]

A solution of tert-butyl 2-{2-cyano-2-[(diphenylmethylidene)amino]ethyl}-6-(3-methyl-2-oxo-1,3-benzoxazol-5-yl)indole-1-carboxylate (1.2 g, 2.01 mmol, 1.0 equiv) in THE (60 mL) was treated with HCl (1M) (3 mL) dropwise at room temperature. The resulting mixture was stirred for 4 h at room temperature. The resulting mixture was diluted with water (50 mL). The resulting mixture was washed with 2×50 mL of diethyl ether. The residue was basified to pH 10 with saturated NaHCO₃ (aq.). The resulting mixture was extracted with EtOAc (3×30 mL). The combined organic layers were washed with brine (1×50 mL), dried over anhydrous Na₂SO₄. After filtration, the filtrate was concentrated under reduced pressure. The crude product 2-amino-3-[6-(3-methyl-2-oxo-1,3-benzoxazol-5-yl)-1H-indol-2-yl]propanenitrile (480 mg, 71.81%) as a brown solid was used in the next step directly without further purification. LCMS (ES, m/z): [M+H]⁺: 433.

### Synthesis of tert-butyl (2S)-2-((2-(1-(tert-butoxycarbonyl)-6-(3-methyl-2-oxo-2,3-dihydrobenzo[d]oxazol-5-yl)-1H-indol-2-yl)-1-cyanoethyl)carbamoyl)-1,4-oxazepane-4-carboxylate

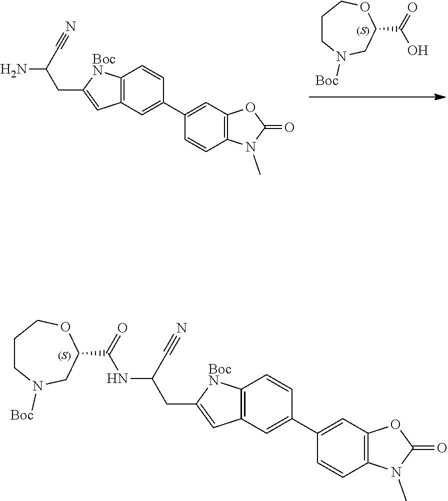

A solution of 2-amino-3-[6-(3-methyl-2-oxo-1,3-benzoxazol-5-yl)-1H-indol-2-yl]propanenitrile (258 mg, 0.78 mmol, 1.0 equiv) in DCM (3 mL) was treated with (2S)-4-(tert-butoxycarbonyl)-1,4-oxazepane-2-carboxylic acid (190.40 mg, 0.78 mmol, 1 equiv) and DIEA (300.99 mg, 2.33 mmol, 3.0 equiv) at room temperature followed by the addition of HATU (354.19 mg, 0.93 mmol, 1.2 equiv) in portions at 0° C. The resulting mixture was stirred for 4 h at room temperature. The resulting mixture was concentrated under reduced pressure. The residue was purified by silica gel column chromatography, eluted with PE/EA (1:1) to afford tert-butyl (2S)-2-({1-cyano-2-[6-(3-methyl-2-oxo-1,3-benzoxazol-5-yl)-1H-indol-2-yl]ethyl}carbamoyl)-1,4-oxazepane-4-carboxylate (238 mg, 54.79%) as a colorless foam. LCMS (ES, m/z): [M+H]⁺: 660.

### Synthesis of (2S)—N-(1-cyano-2-(6-(3-methyl-2-oxo-2,3-dihydrobenzo[d]oxazol-5-yl)-1H-indol-2-yl)ethyl)-1,4-oxazepane-2-carboxamide

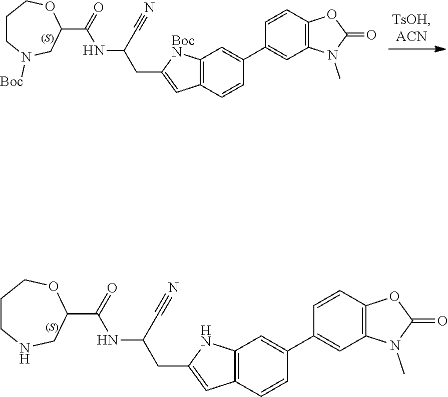

A solution of tert-butyl 2-(2-{[(2S)-4-(tert-butoxycarbonyl)-1,4-oxazepan-2-yl]formamido}-2-cyanoethyl)-6-(3-methyl-2-oxo-1,3-benzoxazol-5-yl)indole-1-carboxylate (230 mg, 0.35 mmol, 1 equiv) in ACN (3 mL) was treated with TsOH (600.33 mg, 3.49 mmol, 10 equiv) at room temperature. The resulting mixture was stirred for overnight at room temperature. The resulting mixture was purified by HPLC to afford (2S)—N-{1-cyano-2-[6-(3-methyl-2-oxo-1,3-benzoxazol-5-yl)-1H-indol-2-yl]ethyl}-1,4-oxazepane-2-carboxamide (60 mg, 37.45%) as a white solid.

### Analytical Data

LCMS (ES, m/z): [M+H]⁺: 460.2

¹H NMR (300 MHz, DMSO-d₆) δ 11.15 (d, J=7.8 Hz, 1H), 8.93-8.48 (m, 1H), 7.66-7.51 (m, 3H), 7.46-7.35 (m, 2H), 7.32 (d, J=8.3 Hz, 1H), 6.37-6.31 (m, 1H), 5.29-5.09 (m, 1H), 4.15-3.80 (m, 2H), 3.83-3.62 (m, 1H), 3.43 (d, J=2.1 Hz, 3H), 3.40-3.36 (m, 1H), 3.22-3.02 (m, 2H), 2.85-2.57 (m, 3H), 1.85-1.62 (m, 2H).

### Example 18. Synthesis of (2S)—N-(1-cyano-2-(3-(4-cyanophenyl)bicyclo[1.1.1]pentan-1-yl)ethyl)-1,4-oxazepane-2-carboxamide (Compound 136b)

### Synthesis of 4-(3-(2-amino-2-cyanoethyl)bicyclo[1.1.1]pentan-1-yl)benzonitrile

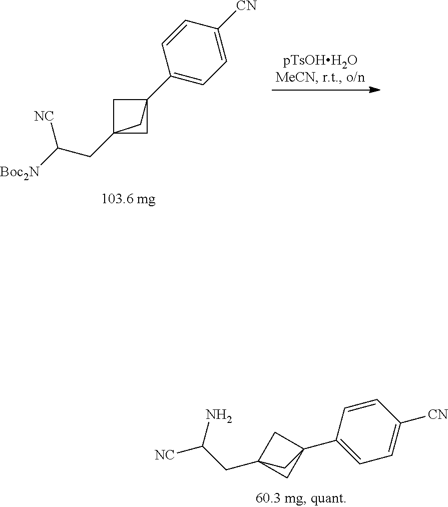

Into a round bottom flask were added tert-butyl (tert-butoxycarbonyl)(1-cyano-2-(3-(4-cyanophenyl)bicyclo[1.1.1]pentan-1-yl)ethyl)carbamate (103.6 mg, 1 Eq, 236.8 mol), MeCN (4.736 mL) and 4-methylbenzenesulfonic acid hydrate (135.1 mg, 3 Eq, 710.3 mol), at 23° C. The reaction mixture was cooled by ice bath and basified to 8 with saturated NaHCO₃ (aq.), extracted with ethyl acetate (30 mL×3). The combined organic layers were washed with brine (100 mL), dried over Na₂SO₄. After filtration, the filtrate was concentrated under reduced pressure. This resulted in 4-(3-(2-amino-2-cyanoethyl)bicyclo[1.1.1]pentan-1-yl)benzonitrile (60.3 mg crude). The product was used without further purification in the next step.

### tert-butyl (2S)-2-((1-cyano-2-(3-(4-cyanophenyl)bicyclo[1.1.1]pentan-1-yl)ethyl)carbamoyl)-1,4-oxazepane-4-carboxylate

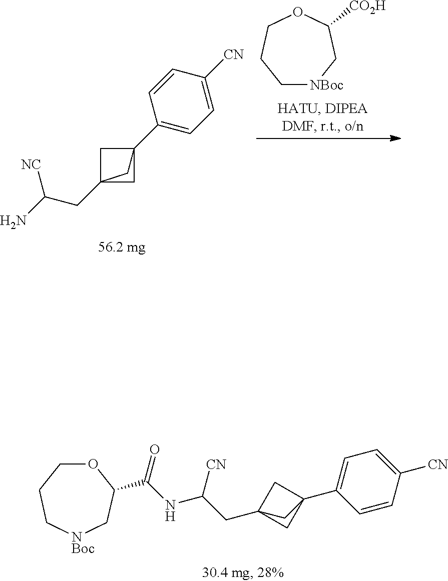

To the mix of 4-(4-(2-amino-2-cyanoethyl)bicyclo[1.1.1]pentan-1-yl)benzonitrile (56.20 mg, 1 Eq, 236.8 mol), (S)-4-(tert-butoxycarbonyl)-1,4-oxazepane-2-carboxylic acid (60.98 mg, 1.05 Eq, 248.6 mol) and HATU (108.0 mg, 1.2 Eq, 284.2 mol) dry DMF (1.850 mL) was added, followed by diisopropylethylamine (91.82 mg, 123 μL, 3.0 Eq, 710.4 mol). The reaction mixture was stirred under N2 for 16 h at 23° C. The reaction mixture was diluted with 30 mL of ethyl acetate, transferred to a separatory funnel, washed with 20 mL of water+1 mL of 2N KHSO₄, then with 20 mL of an aqueous solution of NaHCO₃, and brine 4×20 mL. The organic layers were combined and dried over Na₂SO₄, filtered and concentrated under reduced pressure to give crude product.

The crude was purified by flash column chromatography (CyH/EA) to afford tert-butyl (2S)-2-((1-cyano-2-(3-(4-cyanophenyl)bicyclo[1.1.1]pentan-1-yl)ethyl)carbamoyl)-1,4-oxazepane-4-carboxylate (30.4 mg, 28%).

¹H NMR (400 MHz, CDCl3) δ 7.59 (d, J=8.2 Hz, 2H), 7.31-7.27 (m, 2H), 4.91 (s, 1H), 4.29-3.96 (m, 4H), 3.71 (s, 1H), 3.60-3.40 (m, 2H), 3.33 (s, 1H), 2.11 (d, J=3.0 Hz, 8H), 1.97 (s, 2H), 1.47 (d, J=6.6 Hz, 10H).

### (2S)—N-(1-cyano-2-(3-(4-cyanophenyl)bicyclo[1.1.1]pentan-1-yl)ethyl)-1,4-oxazepane-2-carboxamide

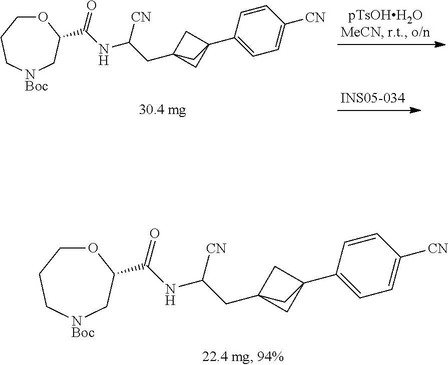 [SMILES: CC(C)(C)OC(=O)N1CCCO[C@H](C(=O)NC(C#N)CC23CC(c4ccc(C#N)cc4)(C2)C3)C1 ; CC(C)(C)OC(=O)N1CCCO[C@H](C(=O)NC(C#N)CC23CC(c4ccc(C#N)cc4)(C2)C3)C1]

Into a round bottom flask were added tert-butyl (2S)-2-((1-cyano-2-(3-(4-cyanophenyl)bicyclo[1.1.1]pentan-1-yl)ethyl)carbamoyl)-1,4-oxazepane-4-carboxylate (30.4 mg, 1 Eq, 65.4 mol), MeCN (1.31 mL) and 4-methylbenzenesulfonic acid hydrate (37.3 mg, 3 Eq, 196 mol), at 23° C. The reaction mixture was cooled with an ice bath, basified to 8 with saturated NaHCO₃ (aq.), poured in a separating funnel and extracted with ethyl acetate (30 mL×3). The combined organic layers were washed with brine (100 mL), and dried over Na₂SO₄. After filtration, the filtrate was concentrated under reduced pressure to afford tert-butyl (2S)-2-((1-cyano-2-(3-(4-cyanophenyl)bicyclo[1.1.1]pentan-1-yl)ethyl)carbamoyl)-1,4-oxazepane-4-carboxylate (22.4 mg, 94%).

¹H NMR (400 MHz, DMSO) δ 8.58 (dd, J=10.9, 8.3 Hz, 1H), 7.79-7.73 (m, 2H), 7.41-7.36 (m, 2H), 4.74 (p, J=7.8 Hz, 1H), 4.01 (ddd, J=17.0, 8.1, 3.6 Hz, 1H), 3.95-3.87 (m, 1H), 3.73 (dtd, J=12.2, 8.0, 4.1 Hz, 1H), 3.17 (dt, J=14.1, 3.7 Hz, 1H), 2.87-2.63 (m, 3H), 2.13 (dd, J=7.3, 2.8 Hz, 2H), 1.98 (d, J=3.8 Hz, 6H), 1.75 (d, J=15.1 Hz, 3H).

### Example 19. Synthesis of 4-(4-cyanophenyl)bicyclo[2.2.2]octane-1-carboxylic acid methyl 4-bromobicyclo[2.2.2]octane-1-carboxylate

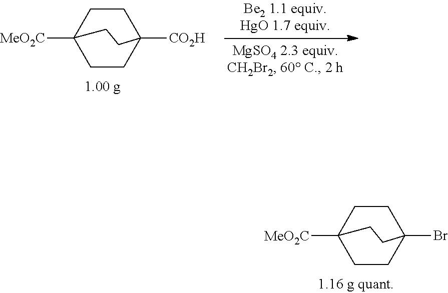 [SMILES: COC(=O)C12CCC(C(=O)O)(CC1)CC2 ; COC(=O)C12CCC(Br)(CC1)CC2]

A flask was charged with Magnesium sulfate (1.304 g, 620.2 μL, 2.3 Eq, 10.84 mmol), 4-(methoxycarbonyl)bicyclo[2.2.2]octane-1-carboxylic acid (1000 mg, 1 Eq, 4.712 mmol), Mercuric oxide, red (1.735 g, 155.7 μL, 1.7 Eq, 8.010 mmol) and CH₂Br₂ (9.982 mL). Bromine (828.3 mg, 316.4 μL, 1.1 Eq, 5.183 mmol) was added dropwise. A condenser was attached and the reaction mixture was stirred at 80° C. for 2 hours. The reaction mixture was allowed to cool to room temperature, filtered on a pad of Celite. The cake was washed with DCM. The filtrate was washed with Na₂S₂O₃. The organic phase was dried over Na₂SO₄, filtered and concentrated to dryness. The crude was pure enough to be engaged in the next step without further purification.

¹H NMR (400 MHz, CDCl₃) δ 3.63 (s, 3H), 2.28-2.20 (m, 6H), 2.00-1.91 (m, 6H).

### methyl 4-phenylbicyclo[2.2.2]octane-1-carboxylate

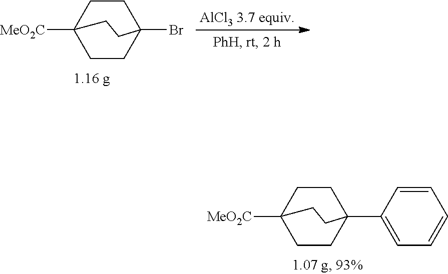 [SMILES: COC(=O)C12CCC(Br)(CC1)CC2 ; COC(=O)C12CCC(c3ccccc3)(CC1)CC2]

To a solution of methyl 4-bromobicyclo[2.2.2]octane-1-carboxylate (1164.0 mg, 1 Eq, 4.7101 mmol) in non dry Benzene (11.405 mL) was added Aluminum chloride (2.3236 g, 802.6 L, 3.7 Eq, 17.427 mmol). Reaction mixture was stirred at rt o/n. Quench with sat. NaHCO₃ and extraction with DCM three times, dry over Na₂SO₄, concentration. Crude NMR: clean. MPLC: Cyclohexane/EtOAc->9:1 yielded methyl 4-phenylbicyclo[2.2.2]octane-1-carboxylate (1.07 g, 4.38 mmol, 93.0%)

¹H NMR (400 MHz, CDCl₃) δ 7.33-7.28 (m, 4H), 7.18 (m, 1H), 3.68 (s, 3H), 1.97-1.82 (m, 12H).

### methyl 4-(4-bromophenyl)bicyclo[2.2.2]octane-1-carboxylate INS02-013

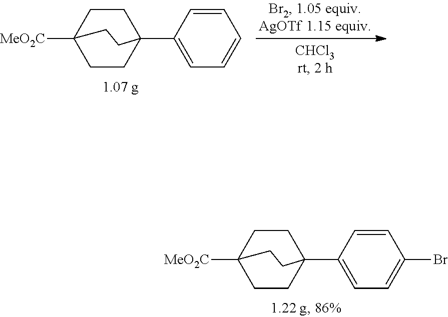 [SMILES: COC(=O)C12CCC(c3ccccc3)(CC1)CC2 ; COC(=O)C12CCC(c3ccc(Br)cc3)(CC1)CC2]

To a stirring solution of methyl 4-phenylbicyclo[2.2.2]octane-1-carboxylate (1.07 g, 13.9 mL, 0.314 molar, 1 Eq, 4.38 mmol) and silver(I) 2,2,2-trifluoroacetate (1.11 g, 1.15 Eq, 5.04 mmol) in CHCl₃ (14 mL) was added drop wise solution of bromine (735 mg, 237 μL, 1.05 Eq, 4.60 mmol) in CHCl₃ (5 mL) and the reaction mixture was stirred at room temperature for 2 h. The reaction mixture was filtered through celite and the bed was washed with CH₂Cl₂. The combined filtrate was concentrated under reduced pressure. The crude residue was purified by Combiflash Isco (Redisep, 40 g Silica, 0 to 10% EtOAc in cyclohexane) to yield methyl 4-(4-bromophenyl)bicyclo[2.2.2]octane-1-carboxylate (1.2179 g, 3.7679 mmol, 86.0%)

¹H NMR (400 MHz, CDCl₃) δ 7.44-7.36 (m, 2H), 7.20-7.14 (m, 2H), 3.67 (s, 3H), 1.94-1.78 (m, 12H).

### 4-(4-bromophenyl)bicyclo[2.2.2]octane-1-carboxylic acid INS02-032

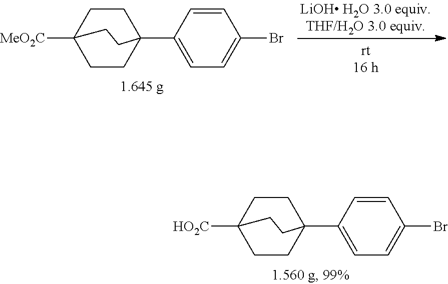 [SMILES: COC(=O)C12CCC(c3ccc(Br)cc3)(CC1)CC2 ; O=C(O)C12CCC(c3ccc(Br)cc3)(CC1)CC2]

To a biphasic solution of methyl 4-(4-bromophenyl)bicyclo[2.2.2]octane-1-carboxylate (1.645 g, 1 Eq, 5.089 mmol) in a mixture of THF (25.45 mL) and H₂O (25.45 mL) was added Lithium hydroxide monohydrate (640.6 mg, 424 μL, 3 Eq, 15.27 mmol). The reaction mixture was stirred at rt for 16 hours. acidification with KHSO₄ 1N, extraction with EtOAc three times. Dried over anhydrous Na₂SO₄, filtered, concentrated to dryness. TLC showed no more SM. Used crude in next step.

### 4-(4-bromophenyl)bicyclo[2.2.2]octane-1-carbonyl chloride

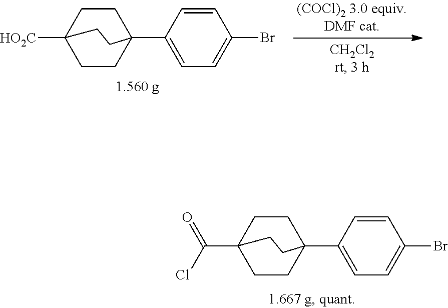 [SMILES: O=C(O)C12CCC(c3ccc(Br)cc3)(CC1)CC2 ; O=C(Cl)C12CCC(c3ccc(Br)cc3)(CC1)CC2]

To a solution of 4-(4-bromophenyl)bicyclo[2.2.2]octane-1-carboxylic acid (1.574 g, 1 Eq, 5.089 mmol) in a mixture of DCM (25.44 mL) was added DMF (18.60 mg, 19.7 μL, 0.05 Eq, 254.5 mol) and slowly oxalyl chloride (1.938 g, 1.336 mL, 3 Eq, 15.27 mmol). Reaction mixture was stirred at rt for 3 hours. Removal of solvent. Assumed quant.

### 4-(4-bromophenyl)bicyclo[2.2.2]octane-1-carboxamide

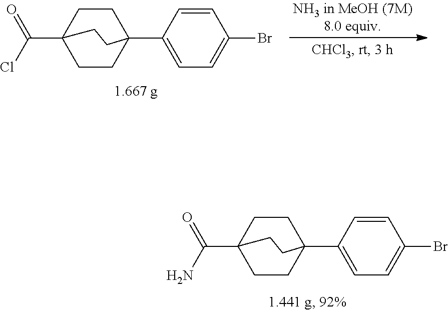 [SMILES: O=C(Cl)C12CCC(c3ccc(Br)cc3)(CC1)CC2 ; NC(=O)C12CCC(c3ccc(Br)cc3)(CC1)CC2]

To a solution of 4-(4-bromophenyl)bicyclo[2.2.2]octane-1-carbonyl chloride (1.667 g, 1 Eq, 5.089 mmol) in CHCl₃ (21.56 mL) was added slowly ammonia in MeOH (693.3 mg, 5.816 mL, 7 molar, 8 Eq, 40.71 mmol). Rxn stirred at rt for 16 hours. Removal of solvent then acidified to pH=1 with HCl 2N. extraction with EtOAc 3 times. Dried over Na₂SO₄, filtered and concentrated to dryness to give 4-(4-bromophenyl)bicyclo[2.2.2]octane-1-carboxamide (1.441 g, 92%).

### 4-(4-bromophenyl)bicyclo[2.2.2]octane-1-carbonitrile INS02-030

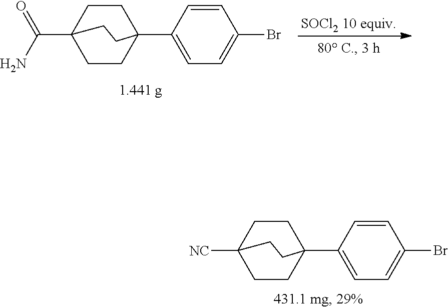 [SMILES: NC(=O)C12CCC(c3ccc(Br)cc3)(CC1)CC2 ; N#CC12CCC(c3ccc(Br)cc3)(CC1)CC2]

4-(4-bromophenyl)bicyclo[2.2.2]octane-1-carboxamide (1.569 g, 1 Eq, 5.089 mmol) was dissolved in Thionyl chloride (6.054 g, 3.742 mL, 10 Eq, 50.89 mmol). This mixture was left to react 3 h at 85° C. Reaction mixture was poured into a saturated. aqueous solution of NaHCO₃, extraction with DCM three times, Na₂SO₄, filtered, concentrated. Purification by FC (SiO₂, Cyclohexane·EtOAc) to afford 4-(4-bromophenyl)bicyclo[2.2.2]octane-1-carbonitrile (431.1 mg, 29%) and MeO₂C-[2.2.2]-Ph-Br (426.5 mg).

¹H NMR (400 MHz, CDCl₃) δ 7.44-7.39 (m, 2H), 7.16-7.11 (m, 2H), 2.10-2.01 (m, 6H), 1.89-1.80 (m, 6H)

### Methyl 4-(4-cyanophenyl)bicyclo[2.2.2]octane-1-carboxylate

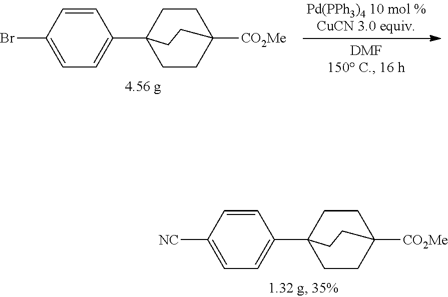 [SMILES: COC(=O)C12CCC(c3ccc(Br)cc3)(CC1)CC2 ; COC(=O)C12CCC(c3ccc(C#N)cc3)(CC1)CC2]

A 20 mL-vial was charged methyl 4-(4-bromophenyl)bicyclo[2.2.2]octane-1-carboxylate (4.560 g, 1 Eq, 14.11 mmol), Tetrakis(triphenylphosphine)palladium(0) (1.630 g, 0.1 Eq, 1.411 mmol), Cuprous cyanide (3.790 g, 1.300 mL, 3 Eq, 42.32 mmol) and DMF (28.22 mL). The resulting solution was purged with N2 for 15 min. The reaction mixture was stirred at 150° C. for 16 hours. It was then allowed to cool to 23° C., addition of sat NaHCO₃. extraction with DCM (*3). Combined organic layers dried over Na₂SO₄, filtered, concentrated to dryness. The residue was purified by MPLC (cyclohexane to cyclohexane:EtOAc 7:3) to afford Methyl 4-(4-cyanophenyl)bicyclo[2.2.2]octane-1-carboxylate (1.32 g, 35%).

¹H NMR (400 MHz, CDCl₃) δ 7.61-7.56 (m, 2H), 7.43-7.39 (m, 2H), 3.68 (s, 3H), 1.97-1.90 (m, 6H), 1.86 (m, 6H).

### 4-(4-cyanophenyl)bicyclo[2.2.2]octane-1-carboxylic acid

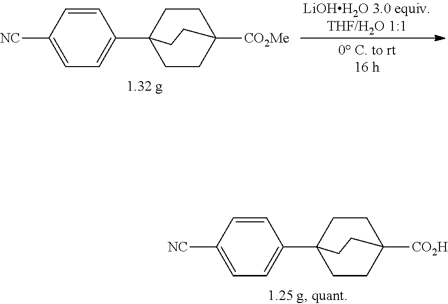 [SMILES: COC(=O)C12CCC(c3ccc(C#N)cc3)(CC1)CC2 ; N#Cc1ccc(C23CCC(C(=O)O)(CC2)CC3)cc1]

To a biphasic solution of methyl 4-(4-cyanophenyl)bicyclo[2.2.2]octane-1-carboxylate (1.32 g, 1 Eq, 4.90 mmol) in a mixture of THE (24.5 mL) and H₂O (24.5 mL) was added Lithium hydroxide monohydrate (617 mg, 409 μL, 3 Eq, 14.7 mmol). The reaction mixture was stirred at 23° C. for 16 hours. Acidification with KHSO₄ 1N, extraction with EtOAc three times. Dried over anhydrous Na₂SO₄, filtered, concentrated to dryness. Crude NMR showed full conv. towards the desired carboxylic acid. Engaged in next step without further purification.

¹H NMR (400 MHz, CDCl₃) δ 7.59 (d, J=8.5 Hz, 2H), 7.42 (d, J=8.4 Hz, 2H), 1.97 (m, 6H), 1.87 (m, 6H).

### Example 20. Synthesis of tert-butyl (tert-butoxycarbonyl)(1-cyano-2-(4-(4-cyanophenyl)bicyclo[2.2.2]octan-1-yl)ethyl)carbamate (Compound 135b)

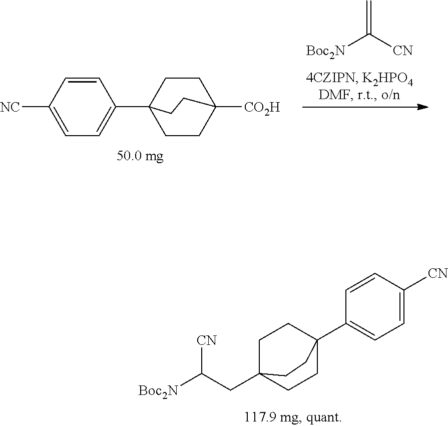

2,4,5,6-tetra(9H-carbazol-9-yl)isophthalonitrile (3.0899 mg, 0.02 Eq, 3.9167 mol), 4-(4-cyanophenyl)bicyclo[2.2.2]octane-1-carboxylic acid (50.000 mg, 1 Eq, 195.83 mol), tert-butyl (tert-butoxycarbonyl)(1-cyanovinyl)carbamate (52.544 mg, 1 Eq, 195.83 mol), and potassium phosphate, dibasic (85.270 mg, 2.5 Eq, 489.58 mol) were added in a small microwave vial, purged with N2 and dry DMF (1.9583 mL) was added. The reaction mixture was purged with N2 for 10 min. The reaction was irradiated with 450 nm Blue LED lamp o/n. The reaction mixture was diluted with a mixture of brine/water 1:1, and the aqueous layer was extracted two times with ethyl acetate. The combined organic layers were washed three times with brine, dried over Na₂SO₄ and concentrated under vacuum. This resulted in tert-butyl (tert-butoxycarbonyl)(1-cyano-2-(4-(4-cyanophenyl)bicyclo[2.2.2]octan-1-yl)ethyl)carbamate (117.9 mg crude). The product was used without further purification in the next step.

¹H NMR (400 MHz, DMSO) δ 7.77-7.71 (m, 2H), 7.55-7.50 (m, 2H), 5.29 (dd, J=7.4, 5.8 Hz, 1H), 1.85-1.52 (m, 13H), 1.49 (s, 15H).

### 4-(4-(2-amino-2-cyanoethyl)bicyclo[2.2.2]octan-1-yl)benzonitrile

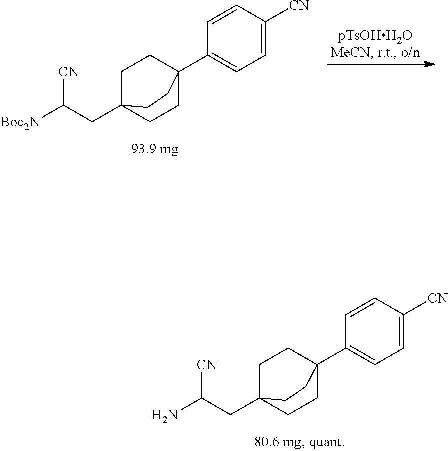

Into a round-bottom flask were added tert-butyl (tert-butoxycarbonyl)(1-cyano-2-(4-(4-cyanophenyl)bicyclo[2.2.2]octan-1-yl)ethyl)carbamate (93.924 mg, 1 Eq, 195.83 mol), MeCN (3.9166 mL) and 4-methylbenzenesulfonic acid hydrate (111.75 mg, 3 Eq, 587.49 mol), at 23° C. The reaction mixture was cooled by ice bath and basified to 8 with saturated NaHCO₃ (aq.), extracted with ethyl acetate (30 mL×3). The combined organic layers were washed with brine (100 mL), dried over Na₂SO₄. After filtration, the filtrate was concentrated under reduced pressure. This resulted in 4-(4-(2-amino-2-cyanoethyl)bicyclo[2.2.2]octan-1-yl)benzonitrile (80.6 mg crude). The product was used without further purification in the next step.

### tert-butyl (2S)-2-((1-cyano-2-(4-(4-cyanophenyl)bicyclo[2.2.2]octan-1-yl)ethyl)carbamoyl)-1,4-oxazepane-4-carboxylate

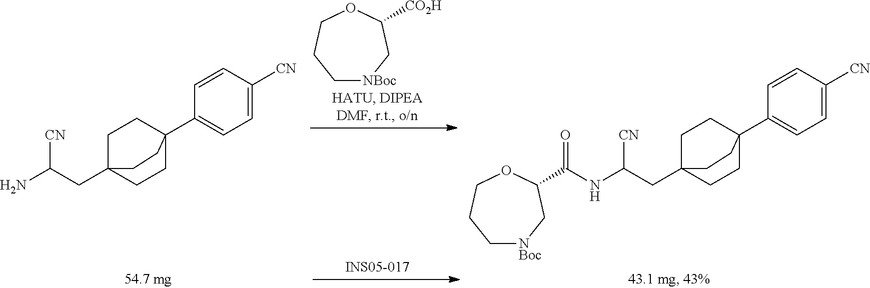

To the mix of 4-(4-(2-amino-2-cyanoethyl)bicyclo[2.2.2]octan-1-yl)benzonitrile (54.713 mg, 1 Eq, 195.83 mol), (S)-4-(tert-butoxycarbonyl)-1,4-oxazepane-2-carboxylic acid (50.433 mg, 1.05 Eq, 205.62 mol) and HATU (89.355 mg, 1.2 Eq, 235.00 mol) dry DMF (1.5299 mL) was added, followed by diisopropylethylamine (75.933 mg, 102 μL, 3.0 Eq, 587.49 mol). The reaction mixture was stirred at 23° C. under N2 for 16 h. The reaction mixture was diluted with 30 mL of ethyl acetate, transferred to a separatory funnel, washed with 20 mL of water+1 mL of 2N KHSO₄, then with 20 mL of an aqueous solution of sat. NaHCO₃, and brine 4×20 mL. The organic layers were combined and dried over Na₂SO₄, filtered and concentrated under reduced pressure to give crude product.

The crude was purified by flash column chromatography (CyH/EA) to afford tert-butyl (2S)-2-((1-cyano-2-(4-(4-cyanophenyl)bicyclo[2.2.2]octan-1-yl)ethyl)carbamoyl)-1,4-oxazepane-4-carboxylate (43.1 mg, 43%).

### (2S)—N-(1-cyano-2-(4-(4-cyanophenyl)bicyclo[2.2.2]octan-1-yl)ethyl)-1,4-oxazepane-2-carboxamide

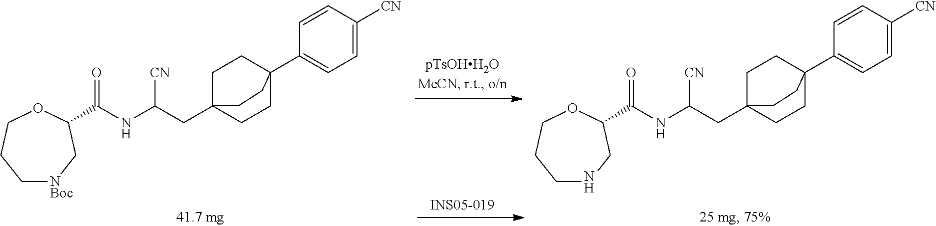 [SMILES: CC(C)(C)OC(=O)N1CCCO[C@H](C(=O)NC(C#N)CC23CCC(c4ccc(C#N)cc4)(CC2)CC3)C1 ; N#Cc1ccc(C23CCC(CC(C#N)NC(=O)[C@@H]4CNCCCO4)(CC2)CC3)cc1]

Into a round-bottom flask were added tert-butyl (2S)-2-((1-cyano-2-(4-(4-cyanophenyl)bicyclo[2.2.2]octan-1-yl)ethyl)carbamoyl)-1,4-oxazepane-4-carboxylate (41.7 mg, 1 Eq, 82.3 mol), MeCN (1.65 mL) and 4-methylbenzenesulfonic acid hydrate (47.0 mg, 3 Eq, 247 mol), at 23° C.

The reaction mixture was cooled with an ice bath, basified to pH=8 with saturated NaHCO₃ (aq.), poured in a separating funnel and extracted with ethyl acetate (30 mL×3). The combined organic layers were washed with brine (100 mL), and dried over Na₂SO₄. After filtration, the filtrate was concentrated under reduced pressure. The crude was purified by flash column chromatography (DCM/MeOH) to afford tert-butyl (2S)-2-((1-cyano-2-(4-(4-cyanophenyl)bicyclo[2.2.2]octan-1-yl)ethyl)carbamoyl)-1,4-oxazepane-4-carboxylate (25 mg, 75%).

¹H NMR (500 MHz, DMSO) δ 8.62 (t, J=8.5 Hz, 1H), 7.76-7.70 (m, 2H), 7.55-7.49 (m, 2H), 4.85-4.71 (m, 1H), 4.08-3.96 (m, 1H), 3.91 (ddd, J=11.9, 10.3, 5.4 Hz, 1H), 3.73 (dddd, J=12.2, 9.8, 8.0, 4.0 Hz, 1H), 3.18 (ddd, J=14.2, 8.6, 3.6 Hz, 1H), 2.85 (tdd, J=13.7, 6.8, 4.6 Hz, 1H), 2.80-2.66 (m, 2H), 1.76 (dq, J=15.6, 5.4 Hz, 10H), 1.52 (q, J=4.7 Hz, 6H).

### Example 21. Synthesis of Intermediates

### tert-butyl (R)-(2-(benzyloxy)-1-cyanoethyl)carbamate

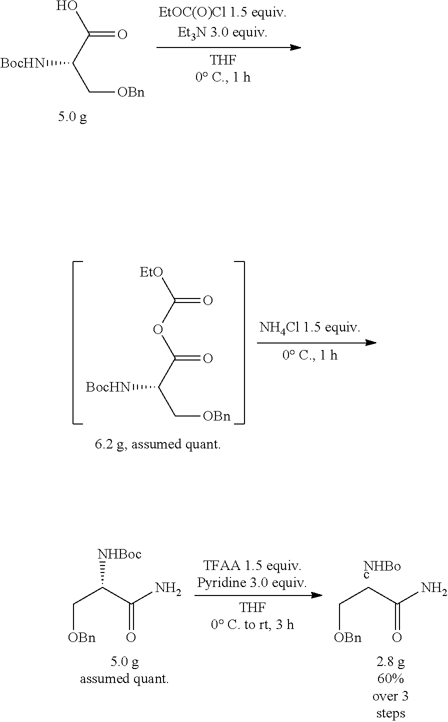

To a solution of O-benzyl-N-(tert-butoxycarbonyl)-L-serine (5.0 g, 1 Eq, 16.930 mmol) in dry THE (338.59 mL), were added at 0° C. triethylamine (5.1393 g, 7.08 mL, 3.000 Eq, 50.789 mmol) and Ethyl chloroformate (2.7558 g, 2.424 mL, 1.5 Eq, 25.394 mmol). The reaction was stirred at rt for 2 hours and used directly in next step.

To a solution of (S)-3-(benzyloxy)-2-((tert-butoxycarbonyl)amino)propanoic (ethyl carbonic) anhydride (6.2201 g, 1 Eq, 16.930 mmol) in dry THE (338.60 mL) was added ammonium chloride aqueous (1.358 g, 25.395 mL, 1.0 molar, 1.5 Eq, 25.395 mmol). The reaction was stirred at rt for 3 hours. Work-up was performed by addition of 1N KHSO₄, extraction with EtOAc three times, then the aqueous phase was dried over Na₂SO₄, filtered and concentrated to dryness. The material was used crude in next step.

Trifluoroacetic anhydride (5.3337 g, 3.546 mL, 1.5 Eq, 25.395 mmol) and pyridine (4.017 g, 4.11 mL, 3 Eq, 50.790 mmol) were added to an ice-cold solution of amide derivative in dry THF (169.30 mL) at 0° C. The resulting reaction mixture was stirred for 2 h. The solvent was removed in vacuo, and the residue was dissolved in ethyl acetate. The organic layer was washed with 1N KHSO₄, water, and brine and dried over anhydrous Na₂SO₄. The crude was purified by FCC (CyH/EtOAc) to afford tert-butyl (R)-(2-(benzyloxy)-1-cyanoethyl)carbamate (2.8 g, 60%).

### tert-butyl (1-cyanovinyl)carbamate

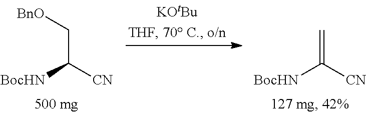 [SMILES: CC(C)(C)OC(=O)N[C@H](C#N)COCc1ccccc1 ; C=C(C#N)NC(=O)OC(C)(C)C]

tert-butyl (R)-(2-(benzyloxy)-1-cyanoethyl)carbamate (500 mg, 1 Eq, 1.81 mmol) and potassium tert butoxide (609 mg, 3 Eq, 5.43 mmol) were dissolved in THF. The reaction mixture was heated at 70° C. for 16 h. The reaction mixture was diluted with EtOAc and water, extracted with EtOAc 3×, dried over Na₂SO₄, and concentrated under vacuum. The crude was purified by FCC (CyH/EtOAc) to afford tert-butyl (1-cyanovinyl)carbamate (127.2 mg, 42%).

¹H NMR (400 MHz, CDCl3) δ 6.27 (s, 1H), 5.88 (s, 1H), 5.37 (dd, J=1.4, 0.6 Hz, 1H).

### tert-butyl (tert-butoxycarbonyl)(1-cyanovinyl)carbamate

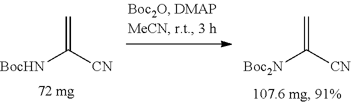

Crude tert-butyl (1-cyanovinyl)carbamate (72 mg, 1 Eq, 0.43 mmol) was dissolved in Acetonitrile (0.86 mL) and DMAP (5.2 mg, 0.1 Eq, 43 mol) and Boc₂O (0.10 g, 0.11 mL, 1.1 Eq, 0.47 mmol) were added. The reaction was stirred for 3 hours at 23° C. The mixture was diluted with EtOAc (50 mL) and washed with brine (40 mL) and 2N KHSO₄ sol. (1 mL). Organic layer was washed again with brine (40 mL) and sat. NaHCO₃ sol. (5 mL). The organic layer was dried over Na₂SO₄ and evaporated to afford crude tert-butyl (tert-butoxycarbonyl)(1-cyanovinyl)carbamate as an orange oil (176.2 mg crude). The crude material was purified by FCC (EtOAc in CyH: 0% for 1 CV, 0% to 100% for 10 CV, 100% for 5 CV) to afford tert-butyl (tert-butoxycarbonyl)(1-cyanovinyl)carbamate (107.6 mg, 0.39 mmol, 91%, 97% Purity).

¹H NMR (400 MHz, CDCl₃) δ 6.05 (d, J=1.5 Hz, 1H), 5.82 (d, J=1.5 Hz, 1H), 1.51 (s, 18H).

### Methyl 3-phenylbicyclo[1.1.1]pentane-1-carboxylate

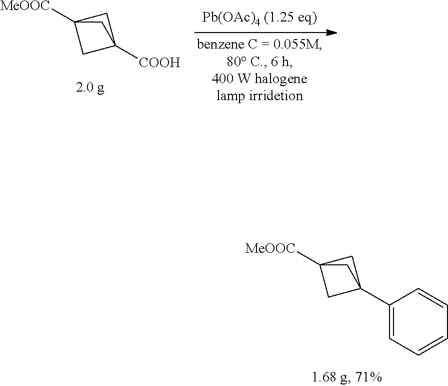 [SMILES: COC(=O)C12CC(C(=O)O)(C1)C2 ; COC(=O)C12CC(c3ccccc3)(C1)C2]

3-(methoxycarbonyl)bicyclo[1.1.1]pentane-1-carboxylic acid (2.00 g, 1 Eq, 11.8 mmol) and lead tetraacetate (6.86 g, 95% Wt, 1.25 Eq, 14.7 mmol) were dissolved in benzene (214 mL), flask was equipped in condenser closed with septum and small N2 balloon (CO₂ releasing). The mixture was irradiated by a 400 W tungsten lamp over 6 h, large quantity of white inorganic precipitate was formed.

After cooling down the reaction flask, white suspension was filtered through short pad (30 g) of celite and a celite pad was washed with a 1:4 ethyl acetate: cyclohexane mix (3×100 mL). The clear colourless filtrate was thereafter concentrated under reduced pressure to give 2.82 g of crude.

Purification by flash column chromatography—80 g [cHex/EA]—0%-1CV, next 0->15%-2CV, and 15%-4CV. To afford methyl 3-phenylbicyclo[1.1.1]pentane-1-carboxylate as colourless oil (1.68 g, 8.31 mmol—71% yield).

TLC: cHex/DCM/EA 90:5:5, Product can be visualised by KMnO₄ stain.

1H NMR (400 MHz, DMSO) δ 7.35-7.29 (m, 2H), 7.28-7.21 (m, 3H), 3.64 (s, 3H), 2.26 (s, 6H).

### 3-Phenylbicyclo[1.1.1]pentane-1-carboxylic acid

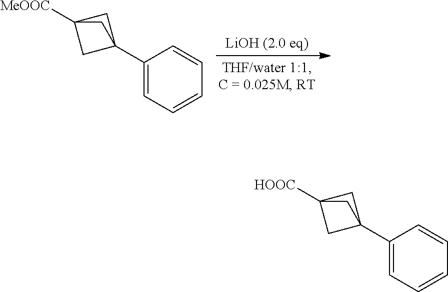 [SMILES: COC(=O)C12CC(c3ccccc3)(C1)C2 ; O=C(O)C12CC(c3ccccc3)(C1)C2]

To a biphasic solution of methyl 3-phenylbicyclo[1.1.1]pentane-1-carboxylate (1600 mg, 1 Eq, 7.911 mmol) in a mixture of THF (79.0 mL) and H₂O (79.0 mL), lithium hydroxide monohydrate (663.9 mg, 2 Eq, 15.82 mmol) was added in one portion. Reaction mixture was stirred at 25° C. for 16 hours. Conversion was determined by LCMS—full conversion. Basic mixture was diluted with 200 mL of water and washed with DCM 2×50 mL. LCMS of organic phase—confirms no product, only impurities. Water phase was acidified with 1M KHSO₄ (20 mL) and extracted with EtOAc (3×100 mL). Combined organics were dried over anhydrous Na₂SO₄, filtered and concentrated to dryness to afford 3-phenylbicyclo[1.1.1]pentane-1-carboxylic acid as white solid (1.31 g, 6.96 mmol—88% yield).

Alternatively:

To a biphasic solution of crude methyl 3-phenylbicyclo[1.1.1]pentane-1-carboxylate (assumed 4750 mg, 1 Eq, 23.50 mmol) in a mixture of THE (235 mL) and H₂O (235 mL), excess of sodium hydroxide (2.82 g, 3 Eq, 70.50 mmol) was added in one portion. Reaction mixture was stirred at 25° C. for 16 hours. Conversion was determined by LCMS—full conversion. Purification as described above, this method leads to 3-phenylbicyclo[1.1.1]pentane-1-carboxylic acid in little bit better total yield (2.96 g, 15.70 mmol—67% yield over two steps).

Product can be visualised by KMnO₄ stain.

1H NMR (400 MHz, DMSO) δ 12.40 (s, 1H), 7.35-7.29 (m, 2H), 7.27-7.20 (m, 3H), 2.21 (s, 6H).

### 3-Phenylbicyclo[1.1.1]pentane-1-carboxamide, MPX-INS01-071/044

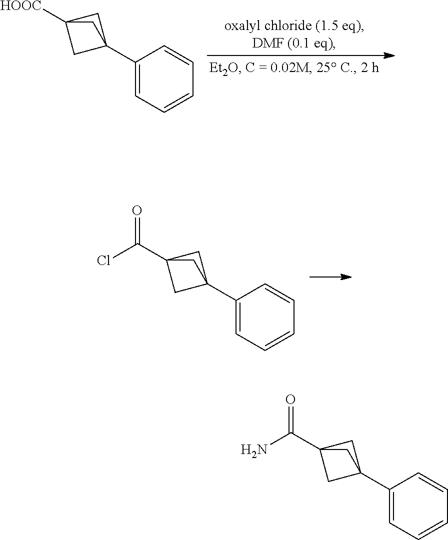 [SMILES: O=C(O)C12CC(c3ccccc3)(C1)C2 ; O=C(Cl)C12CC(c3ccccc3)(C1)C2 ; NC(=O)C12CC(c3ccccc3)(C1)C2]

To a solution of 3-(methoxycarbonyl)bicyclo[1.1.1]pentane-1-carboxylic acid (1430 mg, 1 Eq, 7.597 mmol) and oxalyl chloride (1253 mg, 1.3 Eq, 9.876 mmol, 864.5 L) in Et₂₀ (38 mL), DMF (55 mg, 0.1 Eq, 0.76 mmol, 60 L) as the catalyst was added dropwise. The mixture was stirred around 2 h, until no more gas evolution was observed.

TLC—[EA]->[20 μL sample was taken and quenched with MeOH], visualization by KMnO₄ stain, shows full consumption of SM—formation of less polar spot.

The reaction mixture was concentrated to dryness on rotary evaporator to obtain orange liquid, used crude in next step.

Crude 3-phenylbicyclo[1.1.1]pentane-1-carbonyl chloride (assumed 1.570 g, 1 Eq, 7.597 mmol) was dissolved in DCM (44 mL) and added dropwise to the cold (−20° C.) solution of ammonia in MeOH (5.175 g, 43.5 mL, 7 molar, 40 Eq, 304 mmol) over 15 minutes. The mixture was stirred at −20° C. additional 2 h and allowed to reach 25° C. overnight (16 h).

LCMS shows full conversion of starting material.

The reaction mixture was concentrated under vacuum, and co-evaporated with CH₂Cl₂ twice. The solid was dissolved in DCM (150 mL) and washed with NaHCO₃/brine [2:8](150 mL) and brine (150 mL), dried and evaporated to obtain 3-phenylbicyclo[1.1.1]pentane-1-carboxamide (1.35 g, 7.21 mmol—95% yield) as white solid.

1H NMR (400 MHz, DMSO) δ 7.36-7.27 (m, 3H), 7.27-7.18 (m, 3H), 6.96 (s, 1H), 2.14 (s, 6H).

Alternatively:

Crude 3-phenylbicyclo[1.1.1]pentane-1-carbonyl chloride (assumed 1.437 g, 1 Eq, 6.954 mmol) was dissolved in DCM (70 mL) and cooled down to 0° C., ammonium chloride (1.488 g, 4 Eq, 27.82 mmol) was added in one portion followed by dropwise addition of triethylamine (7.037 g, 9.69 mL, 10 Eq, 69.54 mmol). The mixture was stirred for 2 h in 0° C. and allowed to reach 25° C. overnight, stirring was continued over weekend. LCMS shows a mix of acid and amide. Compounds were separated by 2M HCl/1M NaOH extractions.

### 3-Phenylbicyclo[1.1.1]pentane-1-carbonitrile

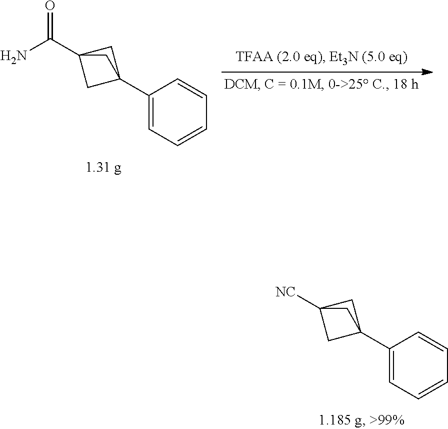 [SMILES: NC(=O)C12CC(c3ccccc3)(C1)C2 ; N#CC12CC(c3ccccc3)(C1)C2]

To an ice-cold solution of 3-phenylbicyclo[1.1.1]pentane-1-carboxamide (1.311 g, 1 Eq, 7.000 mmol) in dry DCM (70.00 mL), triethylamine (3.542 g, 4.88 mL, 5 Eq, 35.00 mmol) was added followed by dropwise addition of trifluoroaceticanhydride (2.940 g, 1.955 mL, 2 Eq, 14.00 mmol). The resulting reaction mixture was allowed to reach 25° C. and stirring was continued over 18 h. After LCMS shows full conversion of starting material flask was cooled by ice bath, and reaction was quenched with NaHCO₃ (30 mL), extracted with DCM (2×50 mL), next the organic layer was washed with 1N KHSO₄ to pH 2, water and brine, organic extract was dried over anhydrous Na₂SO₄.

Purification by flash column chromatography—20 g [pentane/Et₂O]—0%-1CV, next 0->15%-1CV, and 15%-4CV. To afford 3-phenylbicyclo[1.1.1]pentane-1-carbonitrile as colourless oil (1.185 g, 7.00 mmol->99% yield).

Compound seems to be volatile, evaporated max. 350 mBar/40° C.

1H NMR (400 MHz, CDCl3) δ 7.35-7.27 (m, 3H), 7.19-7.15 (m, 2H), 2.51 (s, 6H).

### 5-(4-(3-cyanobicyclo[1.1.1]pentan-1-yl)phenyl)-5H-thianthren-5-ium tetrafluoroborate

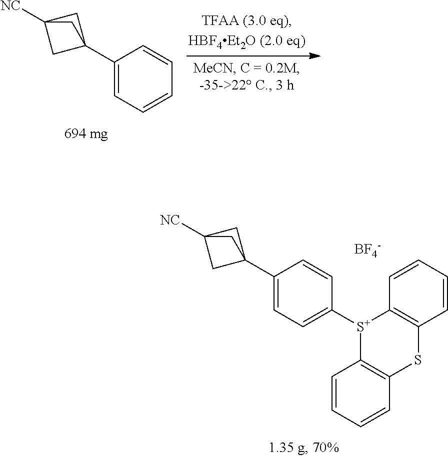 [SMILES: N#CC12CC(c3ccccc3)(C1)C2 ; N#CC12CC(c3ccc([S+]4c5ccccc5Sc5ccccc54)cc3)(C1)C2 ; "BF4-"]

Under an ambient atmosphere, a 50 mL RBF was charged sequentially with 3-phenylbicyclo[1.1.1]pentane-1-carbonitrile (694 mg, 4.10 mmol), thianthrene-S-oxide (1.0 g, 1.05 Eq, 4.30 mmol), and dry MeCN (20.5 ml, c=0.20 M). After most of the solids had dissolved (ca. 5 min), the solution was cooled to −35° C., next TFAA (2.58 g, 1.74 mL, 3.0 Eq, 12.30 mmol) was added in one portion, followed by dropwise addition of tetrafluoroboric acid (1.32 g, 1.11 mL, 2.0 Eq, 8.20 mmol). The mixture was allowed to warm to 23° C. over a period of 1.5 h and stirred in rt for next 1.5 h. LCMS—shows full conversion of SM. Then, the solution was diluted with 150 ml DCM and poured into a separatory funnel. The organic layer was washed with 50 ml of NaHCO₃/100 mL brine and water phase was extracted three times with 50 ml DCM. Combined organic extracts were washed with 150 ml of 10% water solution of NaBF₄ and dried with Na₂SO₄, filtered, and concentrated under reduced pressure.

The crude was purified by column: FCC-12 g, silica, [DCM/MeOH—2CV-100%—DCM, then 4CV 0->10%, and 4CV-10% (Pure product at 8-10% methanol). To obtain product (1.35 g, 2.86 mmol, 70%).

TLC (DCM:MeOH 9:1), Product rf 0.42.

1H NMR (400 MHz, DMSO) δ 8.56 (d, J=6.8 Hz, 2H), 8.05 (dd, J=7.8, 1.2 Hz, 2H), 7.92 (td, J=7.7, 1.5 Hz, 2H), 7.85 (td, J=7.7, 1.4 Hz, 2H), 7.38 (d, J=8.7 Hz, 2H), 7.16 (d, J=8.6 Hz, 2H), 2.50 (s, 6H).

### 5-(4-(3-(methoxycarbonyl)bicyclo[1.1.1]pentan-1-yl)phenyl)-5H-thianthren-5-ium tetrafluoroborate

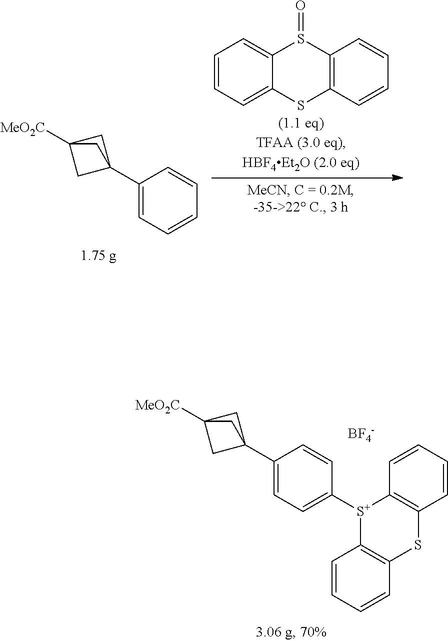 [SMILES: O=S1c2ccccc2Sc2ccccc21 ; COC(=O)C12CC(c3ccccc3)(C1)C2 ; COC(=O)C12CC(c3ccc([S+]4c5ccccc5Sc5ccccc54)cc3)(C1)C2 ; "BF4-"]

Under an ambient atmosphere, a 50 mL RBF was charged sequentially with 3-phenylbicyclo[1.1.1]pentane-1-carbonitrile (1.75 g, 8.65 mmol), thianthrene-S-oxide (2.21 g, 1.10 Eq, 9.52 mmol), and dry MeCN (43.2 ml, c=0.20 M). After most of the solids had dissolved (ca. 5 min), the solution was cooled to −35° C., next TFAA (5.45 g, 3.66 mL, 3.0 Eq, 25.96 mmol) was added in one portion, followed by dropwise addition of tetrafluoroboric acid (2.78 g, 2.35 mL, 2.0 Eq, 17.31 mmol). The mixture was allowed to warm to 23° C. over a period of 1.5 h and stirred in rt for the next 1.5 h. LCMS—shows full conversion of SM. Then, the solution was diluted with 150 ml DCM and poured into a separatory funnel. The organic layer was washed with 50 ml of NaHCO₃/100 mL brine and water phase was extracted three times with 50 ml DCM. Combined organic extracts were washed with 150 ml of 10% water solution of NaBF₄ and dried with Na₂SO₄, filtered, and concentrated under reduced pressure.

The crude was purified by column: FCC-12 g, silica, [DCM/MeOH—2CV-100%—DCM, then 4CV 0->10%, and 4CV-10% (Pure product at 8-10% methanol). To obtain product (3.06 g, 6.08 mmol, 70%).

TLC (DCM:MeOH 9:1), Product rf 0.42.

1H NMR (400 MHz, DMSO) δ 8.56 (dd, J=7.9, 1.3 Hz, 2H), 8.06 (dd, J=7.9, 1.2 Hz, 2H), 7.92 (td, J=7.8, 1.5 Hz, 2H), 7.85 (td, J=7.7, 1.4 Hz, 2H), 7.41 (d, J=8.7 Hz, 2H), 7.16 (d, J=8.6 Hz, 2H), 3.61 (s, 3H), 2.24 (s, 6H).

### Methyl 3-(4-cyanophenyl)bicyclo[1.1.1]pentane-1-carboxylate

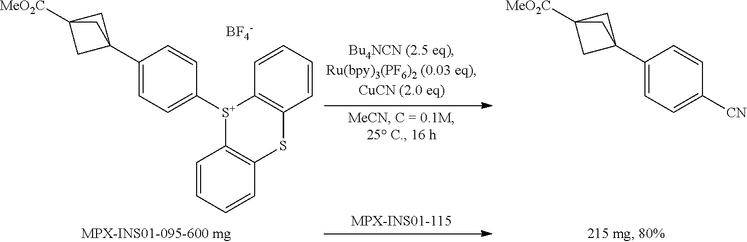 [SMILES: COC(=O)C12CC(c3ccc([S+]4c5ccccc5Sc5ccccc54)cc3)(C1)C2 ; "BF4-" ; COC(=O)C12CC(c3ccc(C#N)cc3)(C1)C2]

Under an ambient atmosphere, a 25 mL RBF was charged sequentially with BCP thianthrenium salt (600 mg, 1 Eq, 1.19 mmol), tetrabutylammonium cyanide (799 mg, 2.5 Eq, 2.97 mmol), Tris(2,2′-bipyridine)ruthenium bis(hexafluorophosphate) (30.7 mg, 0.03 Eq, 35.7 mol), and copper(I) cyanide (213 mg, 2 Eq, 2.38 mmol). Reagents were dissolved/suspended in dry MeCN (12 mL). The mixture was degassed by vacuum/N₂ refiling 3× and submitted to the light irradiation (LEDs 450 nm) for 16 h at 22-25° C. LCMS—shows full conversion of SM. Then, the solution was diluted with 10 ml EA filtered through Celite, filtrate was concentrated under reduced pressure.

The crude was purified by column: FCC-12 g, silica, [cHex/EA—1CV-100%—cHex, then 0.5CV 0->6%, and 3CV-6%, then 1CV 6->20%, and 5CV-20%

To obtain product (215 mg, 0.94 mmol, 80%).

TLC (cHex/EA, 8:2), Product rf 0.5.

1H NMR (400 MHz, DMSO) δ 7.80 (d, J=8.2 Hz, 2H), 7.45 (d, J=8.2 Hz, 2H), 3.64 (s, 3H), 2.32 (s, 6H).

### 3-(4-cyanophenyl)bicyclo[1.1.1]pentane-1-carboxylic acid

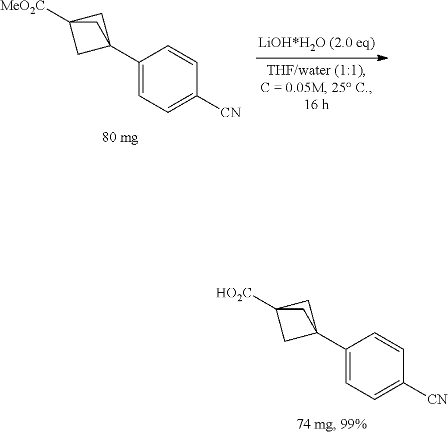 [SMILES: COC(=O)C12CC(c3ccc(C#N)cc3)(C1)C2 ; N#Cc1ccc(C23CC(C(=O)O)(C2)C3)cc1]

To a biphasic solution of methyl 3-(4-cyanophenyl)bicyclo[1.1.1]pentane-1-carboxylate (80.0 mg, 1.0 Eq, 352 mol) in a mixture of THF (3.52 mL) and H₂O (3.52 mL) was added lithium hydroxide hydrate (29.5 mg, 2.0 Eq, 704 mol). Reaction mixture was stirred at 25° C. for 16 hours. Then, the solution was diluted with 50 mL of water, extracted with DCM 2×20 mL, next the water phase was acidified with KHSO₄ 1N, and extracted with DCM 3×20 mL, combined organic extracts were dried over anhydrous Na₂SO₄, filtered, concentrated to dryness. To obtain product (74 mg, 0.35 mmol, 99%).

Product without further purification was used in the next step.

1H NMR (400 MHz, DMSO) δ 12.35 (brs, 1H), 7.80 (d, J=8.4 Hz, 2H), 7.44 (d, J=8.6 Hz, 2H), 2.27 (s, 6H).

### Tert-butyl (tert-butoxycarbonyl)(cyano(3-(4-cyanophenyl)bicyclo[1.1.1]pentan-1-yl)methyl)carbamate

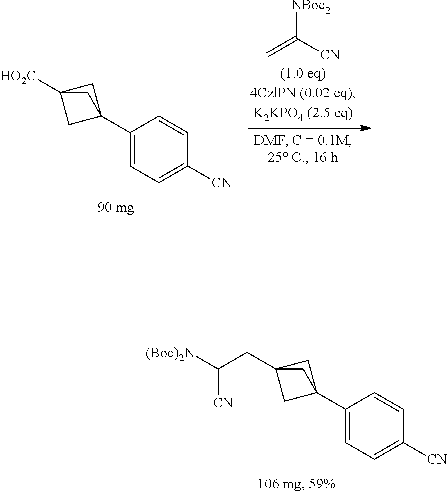

Microwave vial (20 mL) was charged with 3-(4-cyanophenyl)bicyclo[1.1.1]pentane-1-carboxylic acid (90 mg, 1.0 Eq, 422 mol), tert-butyl (tert-butoxycarbonyl)(1-cyanovinyl)carbamate (113 mg, 1.0 Eq, 422 mol), 2,4,5-tri(9H-carbazol-9-yl)-6-(indolin-1-yl)isophthalonitrile (4CzIPN) (6 mg, 0.02 Eq, 8.4 mol), potassium hydrogen phosphate (184 mg, 2.5 Eq, 1.055 mmol) and tightly sealed. Reagents were dissolved/suspended in dry DMF (4.2 mL). The mixture was degassed by vacuum/N₂ refiling 3× and submitted to the light irradiation (LEDs 450 nm) for 16 h at 22-25° C. LCMS—shows partial conversion of SM. Then, the solution was diluted with 50 mL of brine and 2 mL of saturated solution of NaHCO₃, crude solution was extracted with EA (100 mL), organic layer was than washed with brine (3×30 mL),

organic extract was dried over anhydrous Na₂SO₄, filtered, concentrated to dryness.

The crude was purified by column: FCC-4 g, silica, [cHex/EA]

To obtain product (106 mg, 0.24 mmol, 59%). 1H NMR (400 MHz, CDCl3) δ 7.58 (d, J=8.4 Hz, 2H), 7.26 (d, J=8.3 Hz, 2H), 5.29 (t, J=7.6 Hz, 1H), 2.38 (dd, J=14.5, 8.0 Hz, 1H), 2.21 (dd, J=14.4, 7.3 Hz, 1H), 2.13-2.00 (m, 6H), 1.53 (s, 9H), 1.48 (s, 9H).

### Example 22. Synthesis of compound 134c: 2S)—N-{1-cyano-2-[7-fluoro-2-(3-methyl-2-oxo-1,3-benzoxazol-5-yl)-1-benzothiophen-6-yl]ethyl}-1,4-oxazepane-2-carboxamide

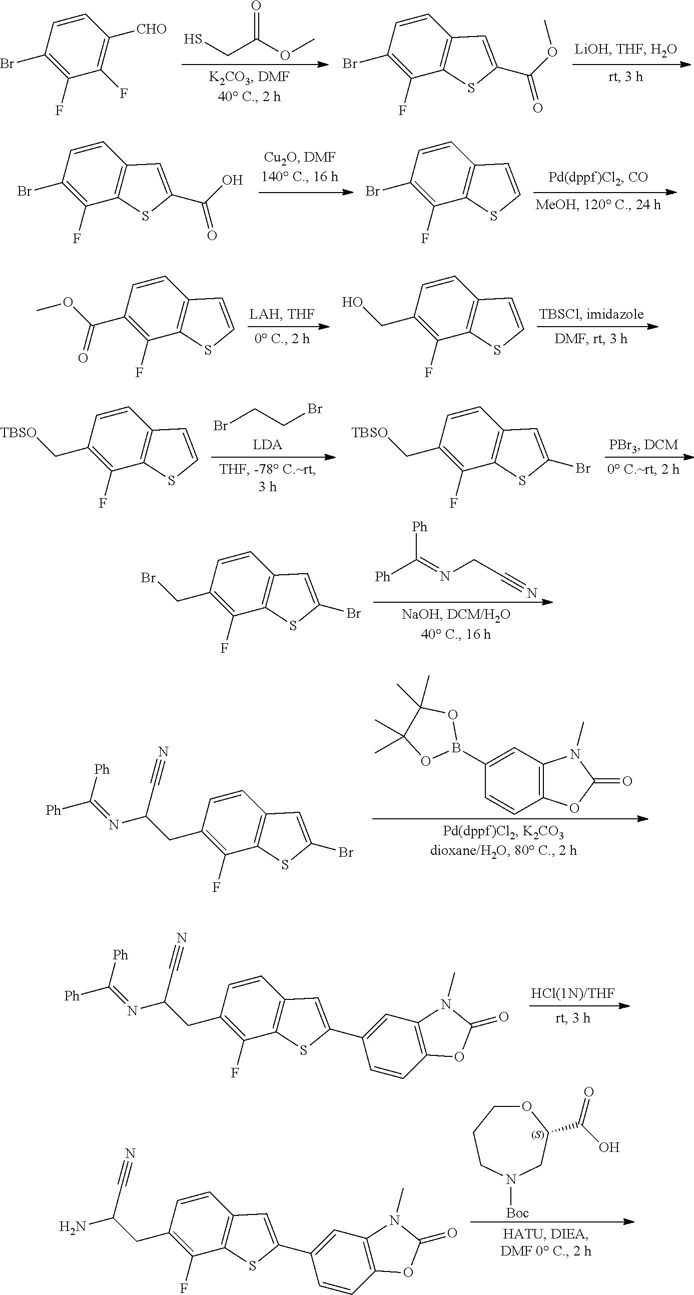

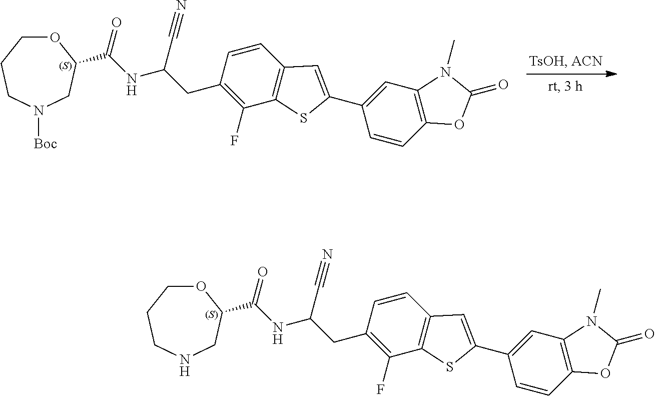

### Experimental Details

### Synthesis of methyl 6-bromo-7-fluoro-1-benzothiophene-2-carboxylate

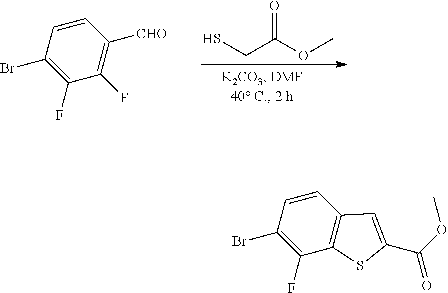

To a stirred mixture of 4-bromo-2,3-difluorobenzaldehyde (20 g, 90.49 mmol, 1.0 equiv) and K₂CO₃ (37.52 g, 271.49 mmol, 3.0 equiv) in DMF (200 mL) was added methyl thioglycolate (14.4 g, 135.74 mmol, 1.5 equiv) at room temperature under nitrogen atmosphere. The resulting mixture was stirred for 2 h at 40° C. under nitrogen atmosphere. The reaction was diluted with water (200 mL), extracted with EtOAc (3×200 mL). The combined organic layer was washed with brine (3×200 mL), dried over anhydrous Na₂SO₄. After filtration, the filtrate was concentrated under reduced pressure. The crude product was re-crystallized from PE/ethyl acetate (5:1, 100 mL) to afford methyl 6-bromo-7-fluoro-1-benzothiophene-2-carboxylate (16.5 g, 63%) as white solid. ¹H NMR (400 MHz, DMSO-d₆) δ 8.32 (d, J=3.6 Hz, 1H), 7.86 (d, J=8.5 Hz, 1H), 7.79 (dd, J=8.5, 6.4 Hz, 1H), 3.92 (s, 3H).

### Synthesis of 6-bromo-7-fluoro-1-benzothiophene-2-carboxylic acid

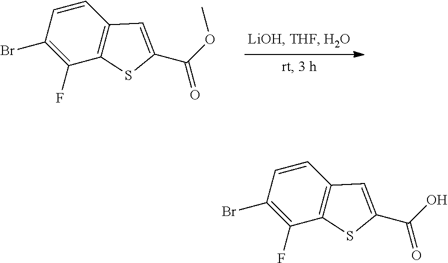

To a stirred solution of methyl 6-bromo-7-fluoro-1-benzothiophene-2-carboxylate (16.5 g, 57.07 mmol, 1.0 equiv) in THE (120 mL) was added LiOH (2.7 g, 114.14 mmol, 2.0 equiv) in H₂O (40 mL) dropwise at room temperature. The resulting mixture was stirred for 3 h at room temperature. The resulting mixture was concentrated under reduced pressure to remove the solvent. The residue was dissolved in water (50 mL), acidified to pH 3 with HCl (aq.). The precipitated solid was collected by filtration and washed with water (2×30 mL). The solid was dried under infrared lamp for 5 h, this resulted in 6-bromo-7-fluoro-1-benzothiophene-2-carboxylic acid (14.5 g, 92%) as white solid.

### Synthesis of 6-bromo-7-fluoro-1-benzothiophene

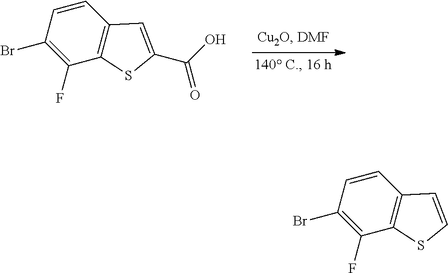 [SMILES: O=C(O)c1cc2ccc(Br)c(F)c2s1 ; Fc1c(Br)ccc2ccsc12]

To a stirred solution of 6-bromo-7-fluoro-1-benzothiophene-2-carboxylic acid (14.5 g, 52.71 mmol, 1.0 equiv) in DMF (120 mL) was added Cu₂O (22.6 g, 158.13 mmol, 3.0 equiv) at room temperature. The resulting mixture was stirred for 16 h at 140° C. The reaction was cooled to room temperature, diluted with water (150 mL) at room temperature. The resulting mixture was extracted with EtOAc (3×200 mL). The combined organic layer was washed with brine (3×300 mL), dried over anhydrous Na₂SO₄. After filtration, the filtrate was concentrated under reduced pressure. The residue was purified by silica gel column chromatography, eluted with PE/EA (10:1) to afford 6-bromo-7-fluoro-1-benzothiophene (10 g, 82%) as white solid.

### Synthesis of methyl 7-fluoro-1-benzothiophene-6-carboxylate

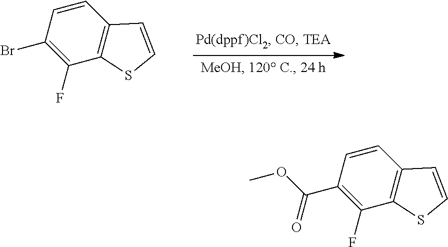

To a 250 mL pressure tank, solution of 6-bromo-7-fluoro-1-benzothiophene (10 g, 43.27 mmol, 1.0 equiv) in MeOH (150 mL) was added TEA (13.1 g, 129.82 mmol, 3.0 equiv), Pd(dppf)Cl₂—CH₂Cl₂ (1.76 g, 2.16 mmol, 0.05 equiv). The mixture was purged with nitrogen for 5 min and then was pressurized to 20 atm with carbon monoxide at 120° C. for 24 h. The reaction mixture was cooled to room temperature and filtered to remove insoluble solid. The resulting mixture was concentrated under reduced pressure. The residue was purified by silica gel column chromatography, eluted with PE/EA (10:1) to afford methyl 7-fluoro-1-benzothiophene-6-carboxylate (6.5 g, 71.5%) as light yellow solid. LCMS (ES, m/z): [M+H]⁺: 211.

### Synthesis of (7-fluoro-1-benzothiophen-6-yl)methanol

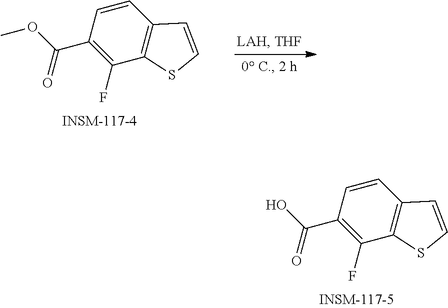

To a stirred solution of methyl 7-fluoro-1-benzothiophene-6-carboxylate (6 g, 28.54 mmol, 1.0 equiv) in THE (70 mL) was added LAH (2 M in THF) (28.5 mL, 57.08 mmol, 2.0 equiv) dropwise at 0° C. under nitrogen atmosphere. The resulting mixture was stirred for 2 h at 0° C. under nitrogen atmosphere. The reaction was quenched with Na₂SO₄₋₁₀H₂O at 0° C. The resulting mixture was filtered, the filter cake was washed with THE (3×50 mL). The resulting filtrate was dried over anhydrous Na₂SO₄. After filtration, the filtrate was concentrated under reduced pressure. This resulted in (7-fluoro-1-benzothiophen-6-yl)methanol (4.5 g, 86.5%) as light yellow oil. LCMS (ES, m/z): [M−H₂O+H]⁺: 165.

### Synthesis of tert-butyl[(7-fluoro-1-benzothiophen-6-yl)methoxy]dimethylsilane

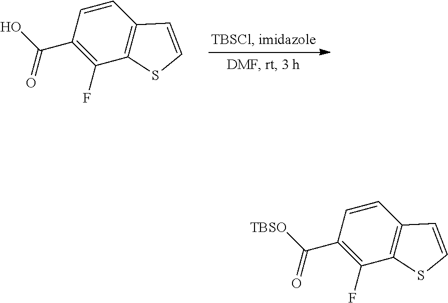 [SMILES: O=C(O)c1ccc2ccsc2c1F ; CC(C)(C)[Si](C)(C)OC(=O)c1ccc2ccsc2c1F]

To a stirred solution of (7-fluoro-1-benzothiophen-6-yl)methanol (4.5 g, 24.69 mmol, 1.0 equiv) and Imidazole (3.36 g, 49.39 mmol, 2.0 equiv) in DMF (60 mL) was added TBS-C₁ (4.47 g, 29.64 mmol, 1.2 equiv) dropwise at room temperature. The resulting mixture was stirred for 3 h at room temperature. The reaction was quenched with water (80 mL) at room temperature. The resulting mixture was extracted with EtOAc (3×150 mL). The combined organic layer was washed with brine (3×200 mL), dried over anhydrous Na₂SO₄. After filtration, the filtrate was concentrated under reduced pressure. The residue was purified by silica gel column chromatography, eluted with PE/EA (20:1) to afford tert-butyl[(7-fluoro-1-benzothiophen-6-yl)methoxy]dimethylsilane (7.0 g, 95.6%) as a light yellow semi-solid. LCMS (ES, m/z): [M-OTBS+H]⁺: 165.

### Synthesis of [(2-bromo-7-fluoro-1-benzothiophen-6-yl)methoxy](tert-butyl)dimethylsilane

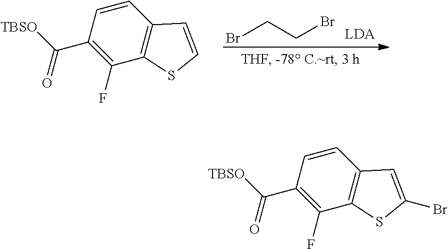

To a stirred solution of tert-butyl[(7-fluoro-1-benzothiophen-6-yl)methoxy]dimethylsilane (4.0 g 13.49 mmol, 1.0 equiv) in THE (50 mL) was added LDA (2 M in THF) (8.77 mL, 17.54 mmol, 1.3 equiv) dropwise at −78° C. under nitrogen atmosphere. The resulting mixture was stirred for 1 h at −78° C. under nitrogen atmosphere. To the above mixture was added dibromoethane (3.29 g, 17.54 mmol, 1.3 equiv) dropwise at −78° C. under nitrogen atmosphere. The resulting mixture was stirred for additional 1 h at room temperature under nitrogen atmosphere. The reaction was quenched by the addition of water (50 mL) at room temperature. The resulting mixture was extracted with EtOAc (3×100 mL). The combined organic layer was dried over anhydrous Na₂SO₄. After filtration, the filtrate was concentrated under reduced pressure. The residue was purified by silica gel column chromatography, eluted with PE/EA (20:1) to afford [(2-bromo-7-fluoro-1-benzothiophen-6-yl)methoxy](tert-butyl)dimethylsilane (3 g, 59%) as a light yellow oil.

### Synthesis of 2-bromo-6-(bromomethyl)-7-fluoro-1-benzothiophene

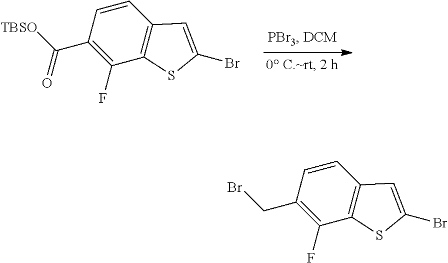 [SMILES: CC(C)(C)[Si](C)(C)OC(=O)c1ccc2cc(Br)sc2c1F ; Fc1c(CBr)ccc2cc(Br)sc12]

To a solution of [(2-bromo-7-fluoro-1-benzothiophen-6-yl)methoxy](tert-butyl)dimethylsilane (2.0 g, 5.32 mmol, 1.0 equiv) in DCM (30 mL) was added PBr₃ (0.72 g, 2.66 mmol, 0.5 equiv) at 0° C. The resulting mixture was stirred for 2 h at room temperature. The reaction was quenched by the addition of water (20 mL) at 0° C. The resulting mixture was extracted with DCM (3×50 mL). The combined organic layer was washed with brine (50 mL), dried over anhydrous Na₂SO₄. After filtration, the filtrate was concentrated under reduced pressure. This resulted in 2-bromo-6-(bromomethyl)-7-fluoro-1-benzothiophene (2.2 g, 89%) as light yellow oil.

### Synthesis of 3-(2-bromo-7-fluorobenzo[b]thiophen-6-yl)-2-((diphenylmethylene)amino)propanenitrile

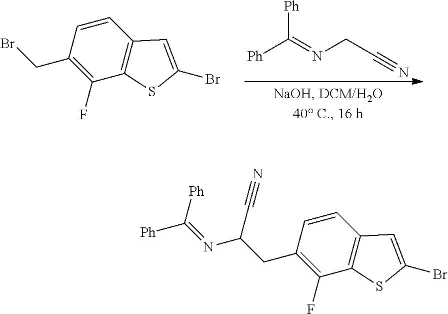 [SMILES: Fc1c(CBr)ccc2cc(Br)sc12 ; N#CCN=C(c1ccccc1)c1ccccc1 ; N#CC(Cc1ccc2cc(Br)sc2c1F)N=C(c1ccccc1)c1ccccc1]

To a solution of 2-bromo-6-(bromomethyl)-7-fluoro-1-benzothiophene (2.2 g, 4.75 mmol, 1.0 equiv) in DCM (20 mL) was added 2-[(diphenylmethylidene)amino]acetonitrile (1.05 g, 4.75 mmol, 1.0 equiv), benzyltrimethylazanium chloride (0.09 g, 0.48 mmol, 0.1 equiv), NaOH (0.38 g, 9.50 mmol, 2.0 equiv) in H₂O (2 mL). The mixture was stirred for 16 h at 40° C. The resulting mixture was diluted with water (20 mL), extracted with CH₂Cl₂ (2×100 mL). The combined organic layer was washed with brine (2×30 mL), dried over anhydrous Na₂SO₄. After filtration, the filtrate was concentrated under reduced pressure. The residue was purified by silica gel column chromatography, eluted with PE/EA (5:1) to afford 3-(2-bromo-7-fluoro-1-benzothiophen-6-yl)-2-[(diphenylmethylidene)amino]propanenitrile (1.2 g, 54.5%) as light yellow solid. LCMS (ES, m/z): [M+H]⁺: 463.

### Synthesis of 2-[(diphenylmethylidene)amino]-3-[7-fluoro-2-(3-methyl-2-oxo-1,3-benzoxazol-5-yl)-1-benzothiophen-6-yl]propanenitrile

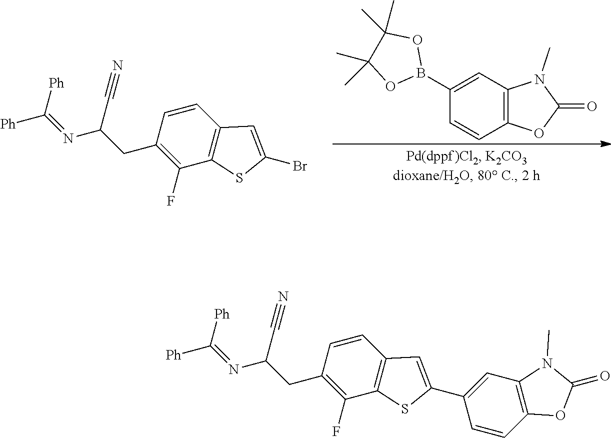 [SMILES: N#CC(Cc1ccc2cc(Br)sc2c1F)N=C(c1ccccc1)c1ccccc1 ; Cn1c(=O)oc2ccc(B3OC(C)(C)C(C)(C)O3)cc21 ; Cn1c(=O)oc2ccc(-c3cc4ccc(CC(C#N)N=C(c5ccccc5)c5ccccc5)c(F)c4s3)cc21]

To a mixture of 3-(2-bromo-7-fluoro-1-benzothiophen-6-yl)-2-[(diphenylmethylidene)amino]propanenitrile (1.2 g, 2.59 mmol, 1.0 equiv), 3-methyl-5-(4,4,5,5-tetramethyl-1,3,2-dioxaborolan-2-yl)benzo[d]oxazol-2(3H)-one (712 mg, 2.59 mmol, 1.0 equiv) in 1,4-dioxane (15 mL), H₂O (1.5 mL), K₂CO₃ (0.72 g, 5.18 mmol, 2.0 equiv) and Pd(dppf)Cl₂—CH₂Cl₂ (0.21 g, 0.26 mmol, 0.1 equiv) were added in sequence. The mixture was stirred for 2 h at 80° C. under nitrogen atmosphere. The reaction was cooled to room temperature, the residue was purified by silica gel column chromatography, eluted with PE/THF (4:1) to afford 2-[(diphenylmethylidene)amino]-3-[7-fluoro-2-(3-methyl-2-oxo-1,3-benzoxazol-5-yl)-1-benzothiophen-6-yl]propanenitrile (1 g, 72.6%) as a light yellow semi-solid. LCMS (ES, m/z): [M+H]⁺: 532.

### Synthesis of 2-amino-3-[7-fluoro-2-(3-methyl-2-oxo-1,3-benzoxazol-5-yl)-1-benzothiophen-6-yl]propanenitrile

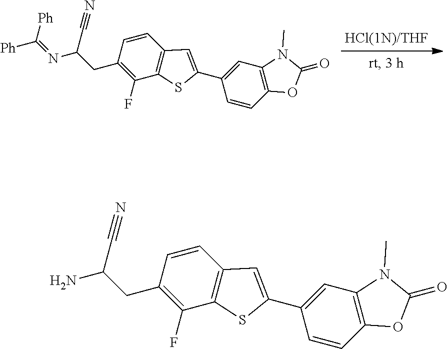 [SMILES: Cn1c(=O)oc2ccc(-c3cc4ccc(CC(C#N)N=C(c5ccccc5)c5ccccc5)c(F)c4s3)cc21 ; Cn1c(=O)oc2ccc(-c3cc4ccc(CC(N)C#N)c(F)c4s3)cc21]

Into a 100 mL round-bottom flask were added 2-[(diphenylmethylidene)amino]-3-[7-fluoro-2-(3-methyl-2-oxo-1,3-benzoxazol-5-yl)-1-benzothiophen-6-yl]propanenitrile (1 g, 1.88 mmol, 1.0 equiv), THE (50 mL), H₂O (5.0 mL) and HCl (1 M) (2.5 mL) at room temperature. The resulting mixture was stirred for 3 h at room temperature. The mixture was basified to pH 12 with NaOH (3 N) The resulting mixture was extracted with EtOAc (3×50 mL). The combined organic layer was washed with brine (3×30 mL), dried over anhydrous Na₂SO₄. After filtration, the filtrate was concentrated under reduced pressure. The residue was purified by silica gel column chromatography, eluted with PE/THF (1:2) to afford 2-amino-3-[7-fluoro-2-(3-methyl-2-oxo-1,3-benzoxazol-5-yl)-1-benzothiophen-6-yl]propanenitrile (480 mg, 69.5%) as white solid. LCMS (ES, m/z): [M+H]⁺: 368.

### Synthesis of tert-butyl (2S)-2-({1-cyano-2-[7-fluoro-2-(3-methyl-2-oxo-1,3-benzoxazol-5-yl)-1-benzothiophen-6-yl]ethyl}carbamoyl)-1,4-oxazepane-4-carboxylate

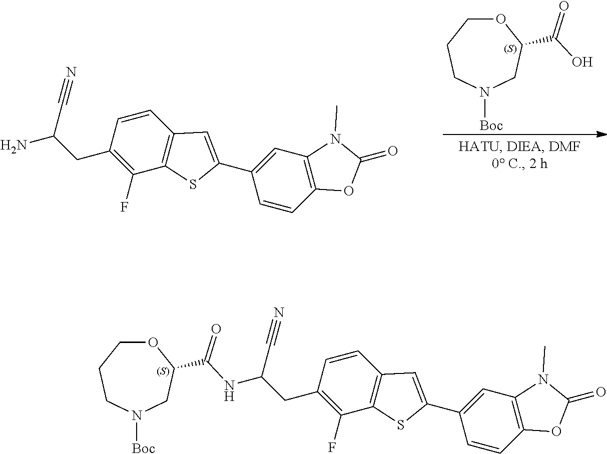

To a stirred mixture of 2-amino-3-[7-fluoro-2-(3-methyl-2-oxo-1,3-benzoxazol-5-yl)-1-benzothiophen-6-yl]propanenitrile (107 mg, 0.29 mmol, 1.2 equiv), (2S)-4-(tert-butoxycarbonyl)-1,4-oxazepane-2-carboxylic acid (60 mg, 0.25 mmol, 1.0 equiv) and DIEA (94 mg, 0.73 mmol, 3.0 equiv) in DMF (5 mL) were added HATU (111 mg, 0.29 mmol, 1.2 equiv) in portions at 0° C. The resulting mixture was stirred for 2 h at 0° C. The reaction was quenched with water (15 mL), extracted with ethyl acetate (30 mL×2), the combined organic phase was washed with brine (20 mL×3), dried over anhydrous sodium sulfate. After filtration, the filtrate was concentrated under reduced pressure. The residue was purified by silica gel column chromatography, eluted with PE/THF (1:1) to afford tert-butyl (2S)-2-({1-cyano-2-[7-fluoro-2-(3-methyl-2-oxo-1,3-benzoxazol-5-yl)-1-benzothiophen-6-yl]ethyl}carbamoyl)-1,4-oxazepane-4-carboxylate (130 mg, 89.4%) as white semi-solid. LCMS (ES, m/z): [M+H]⁺: 595.

### Synthesis of (2S)—N-{1-cyano-2-[7-fluoro-2-(3-methyl-2-oxo-1,3-benzoxazol-5-yl)-1-benzothiophen-6-yl]ethyl}-1,4-oxazepane-2-carboxamide

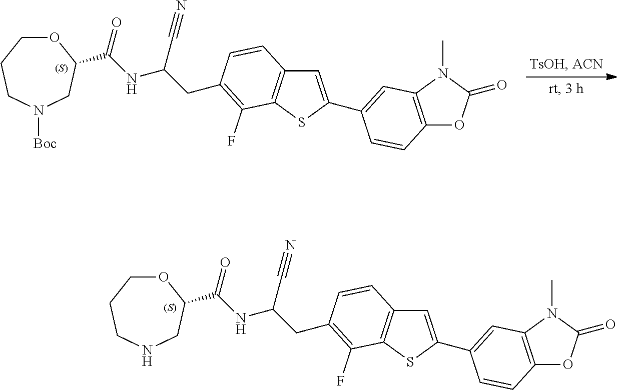

Into a 25 mL round-bottom flask were added tert-butyl (2S)-2-({1-cyano-2-[7-fluoro-2-(3-methyl-2-oxo-1,3-benzoxazol-5-yl)-1-benzothiophen-6-yl]ethyl}carbamoyl)-1,4-oxazepane-4-carboxylate (130 mg, 0.22 mmol, 1.0 equiv), TsOH (113 mg, 0.66 mmol, 3.0 equiv) and ACN (3 mL) at room temperature. The resulting mixture was stirred for 3 h at room temperature. The reaction solution was purified by reversed-phase flash chromatography with the following conditions: column, C18 silica gel-120 g; mobile phase, ACN in Water (0.1% NH₃·H₂O), 10% to 50% gradient in 10 min; detector, UV 254 nm. This resulted in (2S)—N-{1-cyano-2-[7-fluoro-2-(3-methyl-2-oxo-1,3-benzoxazol-5-yl)-1-benzothiophen-6-yl]ethyl}-1,4-oxazepane-2-carboxamide (20 mg, 18.5%) as white solid.

### Analytical Data

LCMS (ES, m/z): [M+H]⁺: 495.

¹H NMR (400 MHz, DMSO-d6) δ 8.72 (dd, J=10.6, 8.5 Hz, 1H), 7.96 (dd, J=3.8, 2.2 Hz, 1H), 7.78 (d, J=1.9 Hz, 1H), 7.67 (dd, J=8.1, 3.2 Hz, 1H), 7.57 (dt, J=8.3, 1.9 Hz, 1H), 7.45 (d, J=8.4 Hz, 1H), 7.41 (td, J=7.9, 7.5, 3.1 Hz, 1H), 5.15-4.97 (m, 1H), 4.03-3.80 (m, 2H), 3.78-3.65 (m, 1H), 3.43 (s, 3H), 3.43-3.35 (m, 1H), 3.33-3.27 (m, 1H), 3.19-2.96 (m, 1H), 2.86-2.54 (m, 4H), 1.79-1.68 (m, 2H).

### Example 23. Synthesis of compound 133b: (2S)—N-{1-cyano-2-[6-(3-methyl-2-oxo-1,3-benzoxazol-5-yl)naphthalen-2-yl]ethyl}-1,4-oxazepane-2-carboxamide

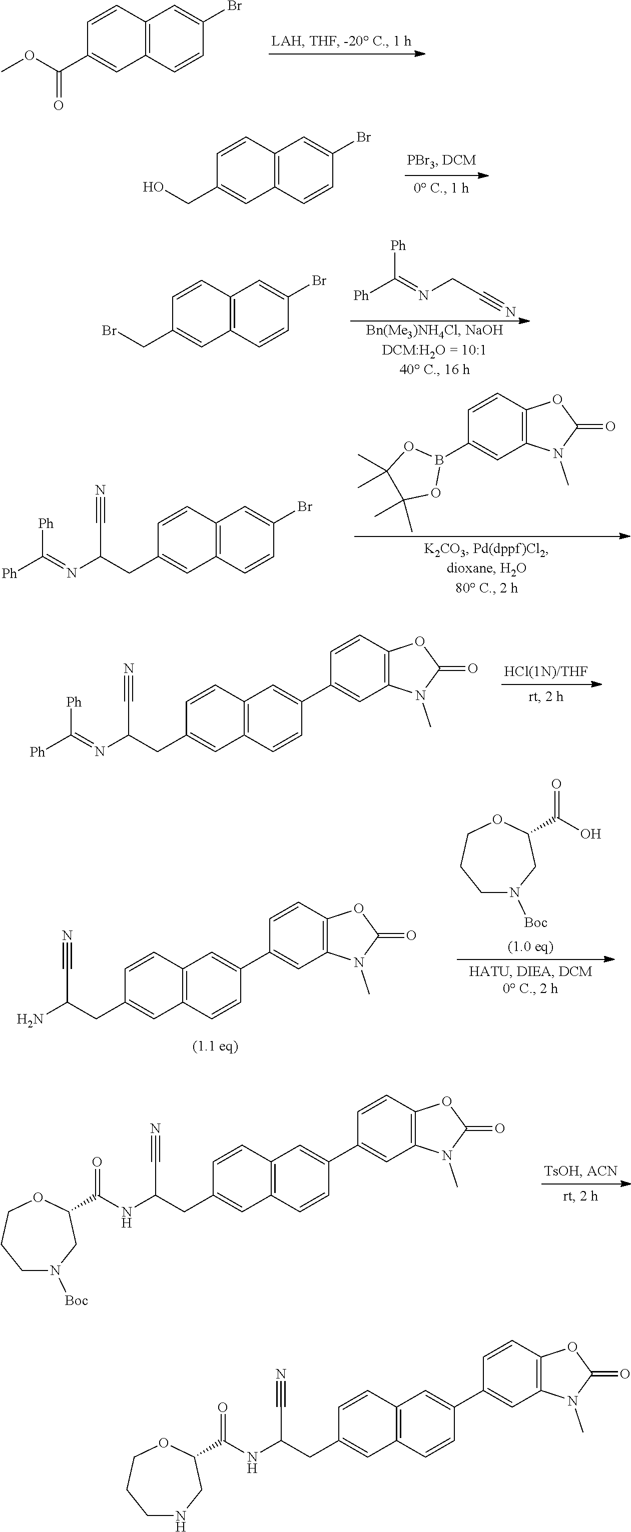

### To Synthesis of (6-bromonaphthalen-2-yl)methanol

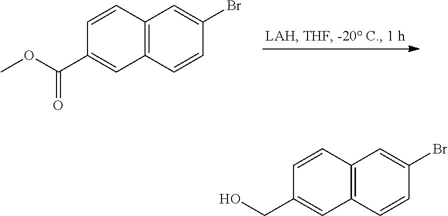

To a stirred solution of methyl 6-bromonaphthalene-2-carboxylate (2.5 g, 9.43 mmol, 1.0 equiv) in THF (50 mL) was added LAH (7.07 mL, 14.14 mmol, 1.5 equiv, 2 M in THF) dropwise at −20° C. under nitrogen atmosphere. The resulting mixture was stirred for 1 h at −20° C. The reaction was quenched with Na₂SO₄·10H₂O solid at room temperature. The resulting mixture was filtered, the filter cake was washed with THF (3×30 mL). The filtrate was concentrated under reduced pressure. The crude product was used in the next step directly without further purification. LCMS (ES, m/z): [M−H₂O+H]⁺: 219.

### Synthesis of 2-bromo-6-(bromomethyl)naphthalene

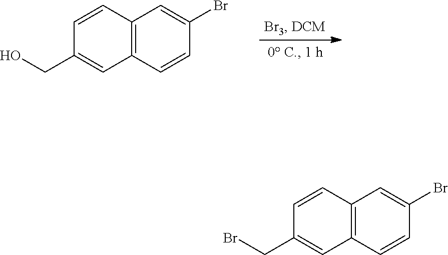 [SMILES: OCc1ccc2cc(Br)ccc2c1 ; BrCc1ccc2cc(Br)ccc2c1]

To a stirred solution of (6-bromonaphthalen-2-yl)methanol (2.5 g, 10.54 mmol, 1.0 equiv) in DCM (50 mL) was added PBr₃ (1.43 g, 5.27 mmol, 0.5 equiv) dropwise at 0° C. under nitrogen atmosphere. The resulting mixture was stirred for 1 h at 0° C. The resulting mixture was quenched with water (100 mL), extracted with CH₂Cl₂ (3×30 mL). The combined organic layer was washed with brine (100 mL), dried over anhydrous Na₂SO₄. After filtration, the filtrate was concentrated under reduced pressure. The resulting mixture was used in the next step directly without further purification (no MS signal in LCMS).

### Synthesis of 3-(6-bromonaphthalen-2-yl)-2-[(diphenylmethylidene)amino]propanenitrile

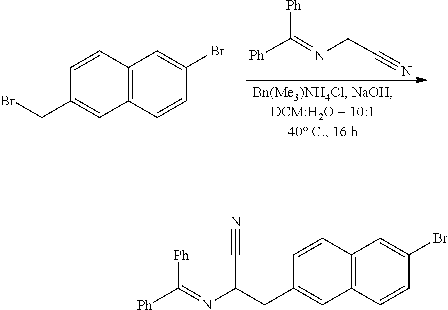

To a stirred mixture of 2-bromo-6-(bromomethyl)naphthalene (2.2 g, 7.33 mmol, 1.0 equiv) and 2-[(diphenylmethylidene)amino]acetonitrile (1.62 g, 7.33 mmol, 1.0 equiv), benzyltrimethylazanium chloride (0.14 g, 0.73 mmol, 0.1 equiv) in DCM (40 mL) was added a solution of NaOH (0.59 g, 14.66 mmol, 2.0 equiv) in H₂O (4 mL) dropwise at room temperature. The resulting mixture was stirred for 16 h at 40° C. The mixture was allowed to cool down to room temperature. The resulting mixture was diluted with water (100 mL), extracted with CH₂Cl₂ (3×30 mL). The combined organic layer was washed with brine (100 mL), dried over anhydrous Na₂SO₄. After filtration, the filtrate was concentrated under reduced pressure. The residue was purified by silica gel column chromatography, eluted with PE/EA (5:1) to afford 3-(6-bromonaphthalen-2-yl)-2-[(diphenylmethylidene)amino]propanenitrile (2 g, 62%) as yellow solid. LCMS (ES, m/z): [M+H]⁺: 439.

### Synthesis of 2-[(diphenylmethylidene)amino]-3-[6-(3-methyl-2-oxo-1,3-benzoxazol-5-yl)naphthalen-2-yl]propanenitrile

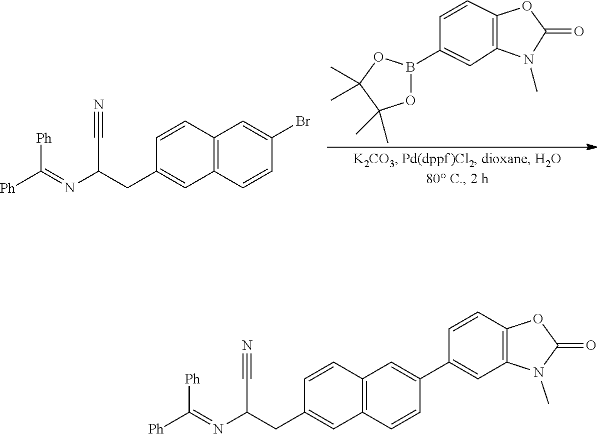

To a stirred mixture of 3-(6-bromonaphthalen-2-yl)-2-[(diphenylmethylidene)amino]propanenitrile (1.1 g, 2.50 mmol, 1.0 equiv) and 3-methyl-5-(4,4,5,5-tetramethyl-1,3,2-dioxaborolan-2-yl)-1,3-benzoxazol-2-one (0.69 g, 2.50 mmol, 1.0 equiv), K₂CO₃ (0.69 g, 5.01 mmol, 2.0 equiv) in dioxane (20 mL) and H₂O (2 mL) was added Pd(dppf)Cl₂ (0.18 g, 0.25 mmol, 0.1 equiv) in portions at room temperature under nitrogen atmosphere. The resulting mixture was stirred for 2 h at 80° C. under nitrogen atmosphere. The mixture was allowed to cool down to room temperature, concentrated under reduced pressure. The residue was purified by silica gel column chromatography, eluted with PE/EA (3:1) to afford 2-[(diphenylmethylidene)amino]-3-[6-(3-methyl-2-oxo-1,3-benzoxazol-5-yl)naphthalen-2-yl]propanenitrile (900 mg, 70.8%) as yellow solid. LCMS (ES, m/z): [M+H]⁺: 508.

### Synthesis of 2-amino-3-[6-(3-methyl-2-oxo-1,3-benzoxazol-5-yl)naphthalen-2-yl]propanenitrile

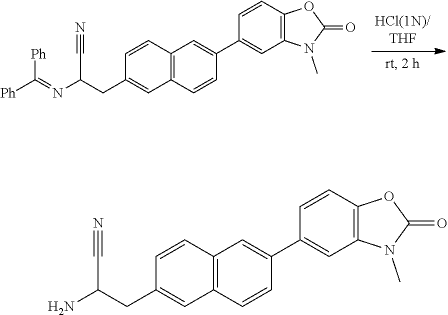 [SMILES: Cn1c(=O)oc2ccc(-c3ccc4cc(CC(C#N)N=C(c5ccccc5)c5ccccc5)ccc4c3)cc21 ; Cn1c(=O)oc2ccc(-c3ccc4cc(CC(N)C#N)ccc4c3)cc21]

To a stirred solution of 2-[(diphenylmethylidene)amino]-3-[6-(3-methyl-2-oxo-1,3-benzoxazol-5-yl)naphthalen-2-yl]propanenitrile (900 mg, 1.77 mmol, 1.0 equiv) in THE (40 mL), H₂O (4 mL) was added HCl (2.2 mL, 1 M) dropwise at room temperature. The resulting mixture was stirred for 2 h at room temperature. The resulting mixture was diluted with water (100 mL), extracted with EtOEt (2×30 mL). The aqueous layer was basified to pH 10 with NaOH (10% in water), extracted with EtOAc (3×50 mL). The combined organic layer was washed with brine (100 mL), dried over anhydrous Na₂SO₄. After filtration, the filtrate was concentrated under reduced pressure. This resulted in 2-amino-3-[6-(3-methyl-2-oxo-1,3-benzoxazol-5-yl)naphthalen-2-yl]propanenitrile (400 mg, 65.7%) as light yellow solid. LCMS (ES, m/z): [M+H]⁺: 344.

### Synthesis of tert-butyl (2S)-2-({1-cyano-2-[6-(3-methyl-2-oxo-1,3-benzoxazol-5-yl)naphthalen-2-yl]ethyl}carbamoyl)-1,4-oxazepane-4-carboxylate

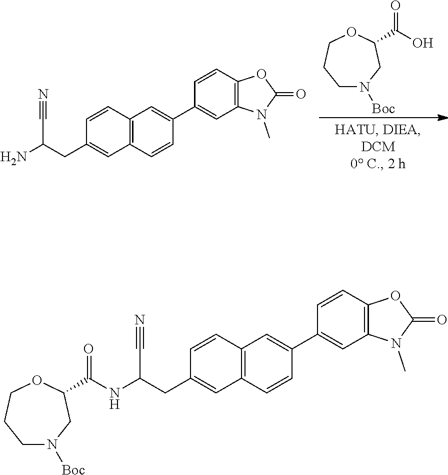

To a stirred solution of 2-amino-3-[6-(3-methyl-2-oxo-1,3-benzoxazol-5-yl)naphthalen-2-yl]propanenitrile (92 mg, 0.27 mmol, 1.1 equiv) and (2S)-4-(tert-butoxycarbonyl)-1,4-oxazepane-2-carboxylic acid (60 mg, 0.24 mmol, 1.0 equiv) in DCM (2 mL) were added DIEA (94 mg, 0.73 mmol, 3.0 equiv) and HATU (111 mg, 0.29 mmol, 1.2 equiv) in portions at 0° C. under nitrogen atmosphere. The resulting mixture was stirred for 2 h at 0° C. under nitrogen atmosphere. Concentrated to remove the solvent, the residue was purified by silica gel column chromatography, eluted with PE/EA (1:1) to afford tert-butyl (2S)-2-({1-cyano-2-[6-(3-methyl-2-oxo-1,3-benzoxazol-5-yl)naphthalen-2-yl]ethyl}carbamoyl)-1,4-oxazepane-4-carboxylate (120 mg, 86%) as a light yellow oil. LCMS (ES, m/z): [M+H]⁺: 571.

### Synthesis of (2S)—N-{1-cyano-2-[6-(3-methyl-2-oxo-1,3-benzoxazol-5-yl)naphthalen-2-yl]ethyl}-1,4-oxazepane-2-carboxamide

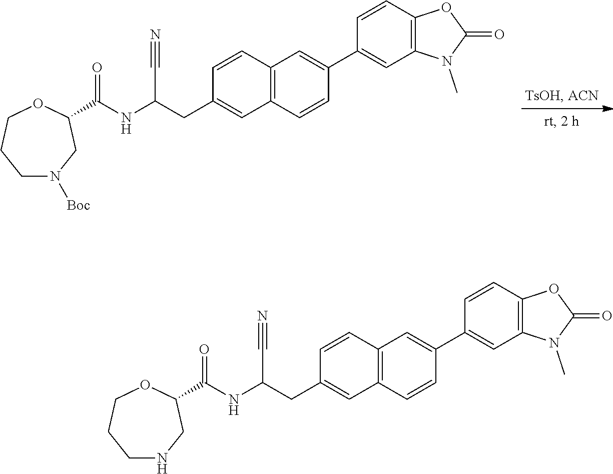 [SMILES: Cn1c(=O)oc2ccc(-c3ccc4cc(CC(C#N)NC(=O)[C@@H]5CN(C(=O)OC(C)(C)C)CCCO5)ccc4c3)cc21 ; Cn1c(=O)oc2ccc(-c3ccc4cc(CC(C#N)NC(=O)[C@@H]5CNCCCO5)ccc4c3)cc21]

To a stirred solution of tert-butyl (2S)-2-({1-cyano-2-[6-(3-methyl-2-oxo-1,3-benzoxazol-5-yl)naphthalen-2-yl]ethyl}carbamoyl)-1,4-oxazepane-4-carboxylate (120 mg, 0.21 mmol, 1.0 equiv) in ACN (3 mL) was added TsOH-H₂O (120 mg, 0.63 mmol, 3.0 equiv) in portions at room temperature. The resulting mixture was stirred for 2 h at room temperature. The reaction solution was purified by reversed-phase flash chromatography with the following conditions: column, C₁₈ silica gel-120 g; mobile phase, MeCN in Water (0.1% NH₃·H₂O), 10% to 70% gradient in 10 min; detector, UV 254 nm. This resulted in (2S)—N-{1-cyano-2-[6-(3-methyl-2-oxo-1,3-benzoxazol-5-yl)naphthalen-2-yl]ethyl}-1,4-oxazepane-2-carboxamide (20 mg, 20%) as white solid.

### Analytical Data

LCMS (ES, m/z): [M+H]⁺: 471.4

¹H NMR (300 MHz, DMSO-d₆) δ 8.67 (t, J=8.8 Hz, 1H), 8.24 (s, 1H), 8.02-7.93 (m, 2H), 7.93-7.86 (m, 1H), 7.83 (s, 1H), 7.75 (d, J=1.8 Hz, 1H), 7.58 (dd, J=8.4, 1.8 Hz, 1H), 7.50 (d, J=8.6 Hz, 1H), 7.46 (d, J=8.3 Hz, 1H), 5.22-4.98 (m, 1H), 4.03-3.62 (m, 3H), 3.45 (s, 3H), 3.37-3.34 (m, 2H), 3.14-2.93 (m, 1H), 2.84-2.61 (m, 2H), 2.46-2.37 (m, 1H), 1.76-1.65 (m, 2H).

### Example 24. Synthesis of compound 116b: (2S)—N-{1-cyano-2-[6-(3-methyl-2-oxo-1,3-benzoxazol-5-yl)-1-benzofuran-2-yl]ethyl}-1,4-oxazepane-2-carboxamide

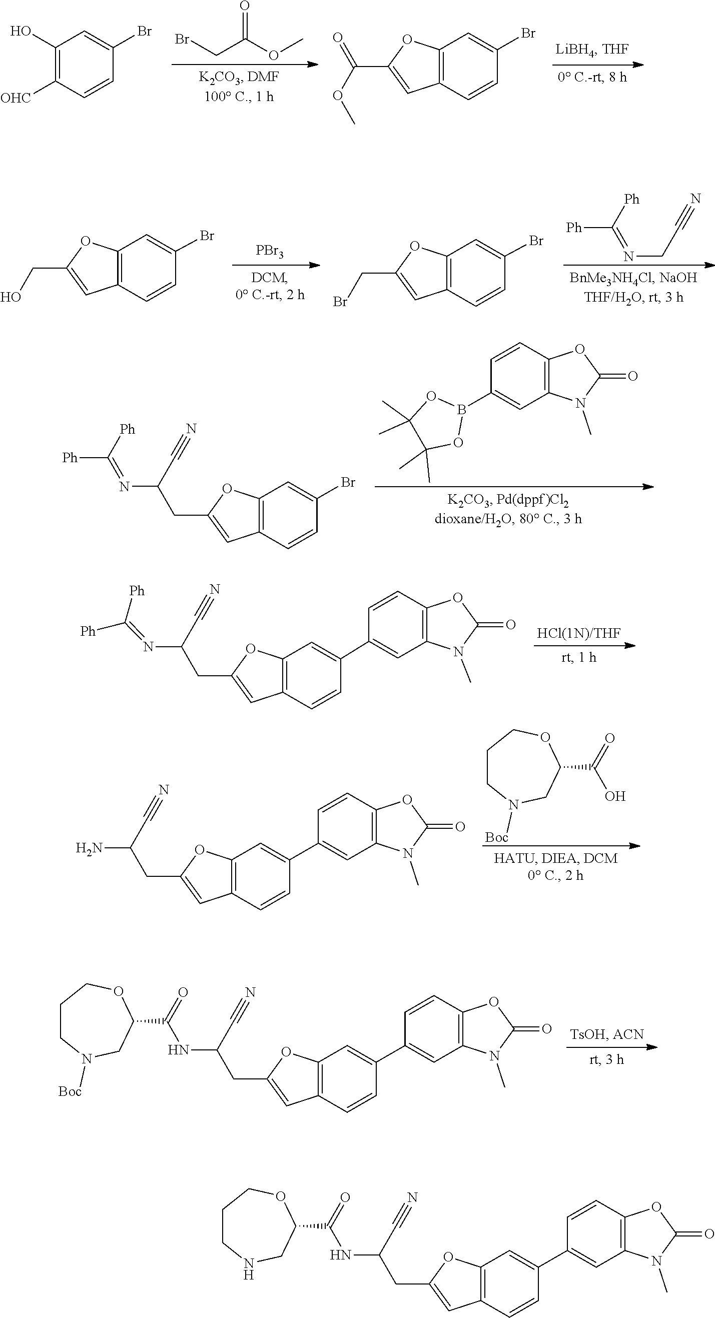

### T Synthesis of methyl 6-bromo-1-benzofuran-2-carboxylate

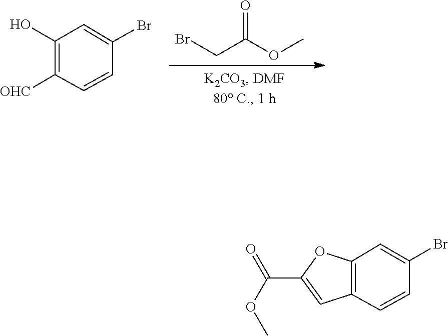

To a stirred solution of 4-bromo-2-hydroxybenzaldehyde (5 g, 24.87 mmol, 1.0 equiv) and K₂CO₃ (10.31 g, 74.61 mmol, 3.0 equiv) in DMF (80 mL) was added methyl 2-bromoacetate (3.80 g, 24.87 mmol, 1.0 equiv) at room temperature under nitrogen atmosphere. The resulting mixture was stirred for 1 h at 100° C. under nitrogen atmosphere. The reaction was cooled to room temperature, diluted with water (100 mL), extracted with EtOAc (3×200 mL). The combined organic layer was washed with brine (3×200 mL), dried over anhydrous Na₂SO₄. After filtration, the filtrate was concentrated under reduced pressure. The crude product was re-crystallized from PE/ethyl acetate (10:1 50 mL) to afford methyl 6-bromo-1-benzofuran-2-carboxylate (2.5 g) as an off-white solid. ¹H NMR (400 MHz, DMSO-d₆) δ 8.08 (d, J=1.7 Hz, 1H), 7.81-7.74 (m, 2H), 7.55 (dd, J=8.4, 1.7 Hz, 1H), 3.90 (s, 3H).

### Synthesis of (6-bromo-1-benzofuran-2-yl)methanol

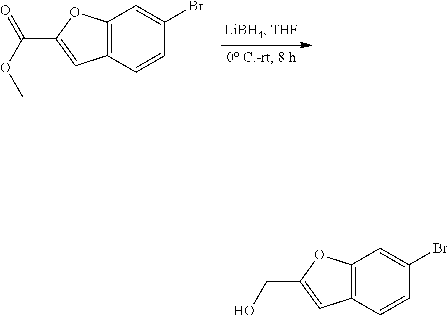

To a stirred solution of methyl 6-bromo-1-benzofuran-2-carboxylate (2.5 g, 9.80 mmol, 1.0 equiv) in THF (30 mL) was added lithium borohydride (2.0 M in THF) (14.7 mL, 29.40 mmol, 3.0 equiv) dropwise at 0° C. under nitrogen atmosphere. The resulting mixture was stirred for 8 h at room temperature. The reaction was quenched with water (20 mL) at 0° C., extracted with EtOAc (3×50 mL). The combined organic layer was dried over anhydrous Na₂SO₄. After filtration, the filtrate was concentrated under reduced pressure. After filtration, the filtrate was concentrated under reduced pressure. The residue was purified by silica gel column chromatography, eluted with PE/EA (3:1) to afford (6-bromo-1-benzofuran-2-yl)methanol (2 g) as a light yellow oil. ¹H NMR (400 MHz, DMSO-d₆) δ 7.85 (s, 1H), 7.56 (d, J=8.3 Hz, 1H), 7.39 (dd, J=8.3, 1.7 Hz, 1H), 6.79 (d, J=1.1 Hz, 1H), 5.52 (t, J=5.9 Hz, 1H), 4.57 (d, J=5.8 Hz, 2H).

### Synthesis of 6-bromo-2-(bromomethyl)-1-benzofuran

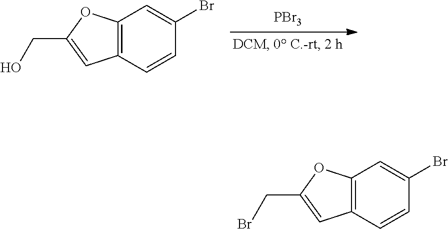 [SMILES: OCc1cc2ccc(Br)cc2o1 ; "PBr3" ; BrCc1cc2ccc(Br)cc2o1]

To a solution of (6-bromo-1-benzofuran-2-yl)methanol (2 g, 8.80 mmol, 1.0 equiv) in DCM (30 mL) was added by the addition of PBr₃ (1.19 g, 4.40 mmol, 0.5 equiv) at 0° C. The resulting mixture was stirred for additional 2 h at room temperature. The reaction was quenched by the addition of water (20 mL) at 0° C. The resulting mixture was extracted with DCM (3×50 mL). The combined organic layer was washed with brine (1×50 mL), dried over anhydrous Na₂SO₄. After filtration, the filtrate was concentrated under reduced pressure. This resulted in 6-bromo-2-(bromomethyl)-1-benzofuran (2 g crude) as a yellow oil and used to the next step directly without further purification.

### Synthesis of 3-(6-bromo-1-benzofuran-2-yl)-2-[(diphenylmethylidene)amino]propanenitrile

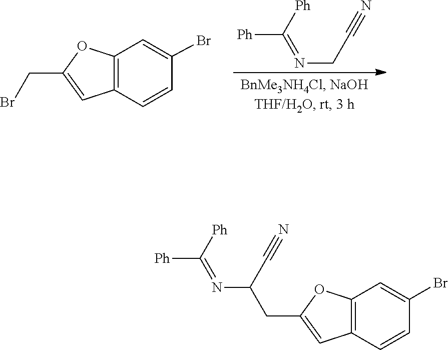 [SMILES: BrCc1cc2ccc(Br)cc2o1 ; N#CCN=C(c1ccccc1)c1ccccc1 ; N#CC(Cc1cc2ccc(Br)cc2o1)N=C(c1ccccc1)c1ccccc1]

To a solution of 6-bromo-2-(bromomethyl)-1-benzofuran (2 g, 6.89 mmol, 1.0 equiv) in THE (30 mL) was added 2-[(diphenylmethylidene)amino]acetonitrile (1.52 g, 6.89 mmol, 1.0 equiv), benzyltrimethylazanium chloride (0.13 g, 0.69 mmol, 0.1 equiv), NaOH (0.55 g, 13.79 mmol, 2.0 equiv) in H₂O (3 mL). The mixture was stirred for 13 h at room temperature. The resulting mixture was extracted with EtOAc (3×50 mL). The combined organic layer was washed with brine (3×30 mL), dried over anhydrous Na₂SO₄. After filtration, the filtrate was concentrated under reduced pressure. The residue was purified by silica gel column chromatography, eluted with PE/EA (20:1) to afford 3-(6-bromo-1-benzofuran-2-yl)-2-[(diphenylmethylidene)amino]propanenitrile (1.2 g) as a yellow oil. LCMS (ES, m/z): [M+H]⁺: 429.

### Synthesis of 2-[(diphenylmethylidene)amino]-3-[6-(3-methyl-2-oxo-1,3-benzoxazol-5-yl)-1-benzofuran-2-yl]propanenitrile

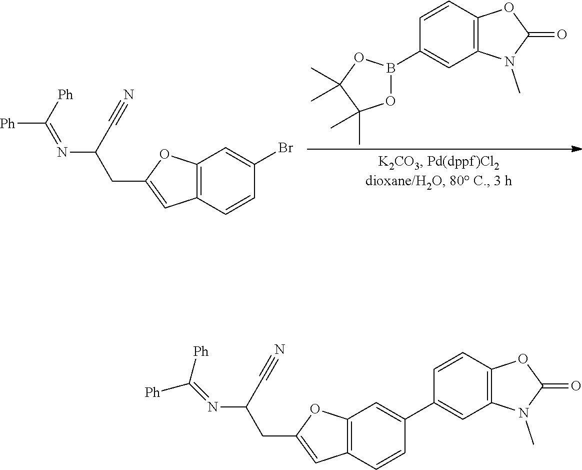 [SMILES: N#CC(Cc1cc2ccc(Br)cc2o1)N=C(c1ccccc1)c1ccccc1 ; Cn1c(=O)oc2ccc(B3OC(C)(C)C(C)(C)O3)cc21 ; Cn1c(=O)oc2ccc(-c3ccc4cc(CC(C#N)N=C(c5ccccc5)c5ccccc5)oc4c3)cc21]

To a solution of 3-(6-bromo-1-benzofuran-2-yl)-2-[(diphenylmethylidene)amino]propanenitrile (900 mg, 2.09 mmol, 1.0 equiv), 3-methyl-5-(4,4,5,5-tetramethyl-1,3,2-dioxaborolan-2-yl)-1,3-benzoxazol-2-one (634 mg, 2.30 mmol, 1.1 equiv) in 1,4-dioxane (15 mL), H₂O (1.5 mL), were added K₂CO₃ (579 mg, 4.19 mmol, 2.0 equiv) and Pd(dppf)Cl₂CH₂Cl₂ (171 mg, 0.21 mmol, 0.1 equiv) in sequence. The mixture was stirred for 3 h at 80° C. under nitrogen atmosphere. The reaction was cooled to room temperature, concentrated to remove the solvent, the residue was purified by silica gel column chromatography, eluted with PE/EA (2:1) to afford 2-[(diphenylmethylidene)amino]-3-[6-(3-methyl-2-oxo-1,3-benzoxazol-5-yl)-1-benzofuran-2-yl]propanenitrile (800 mg, 69%) as a white semi-solid. LCMS (ES, m/z): [M+H]⁺: 498.

### Synthesis of 2-amino-3-[6-(3-methyl-2-oxo-1,3-benzoxazol-5-yl)-1-benzofuran-2-yl]propanenitrile

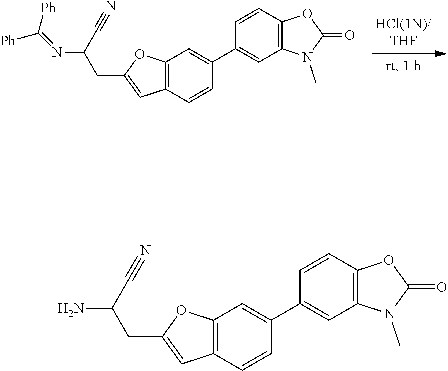 [SMILES: Cn1c(=O)oc2ccc(-c3ccc4cc(CC(C#N)N=C(c5ccccc5)c5ccccc5)oc4c3)cc21 ; Cn1c(=O)oc2ccc(-c3ccc4cc(CC(N)C#N)oc4c3)cc21]

Into a 100 mL round-bottom flask were added 2-[(diphenylmethylidene)amino]-3-[6-(3-methyl-2-oxo-1,3-benzoxazol-5-yl)-1-benzofuran-2-yl]propanenitrile (400 mg, 0.80 mmol, 1.0 equiv), THE (20 mL), H₂O (2 mL) and HCl (1 M) (1 mL) at room temperature. The resulting mixture was stirred for 1 h at room temperature. The mixture was basified to pH 12 with NaOH. The resulting mixture was extracted with EtOAc (3×50 mL). The combined organic layer was washed with brine (3×30 mL), dried over anhydrous Na₂SO₄. After filtration, the filtrate was concentrated under reduced pressure. The residue was purified by silica gel column chromatography, eluted with PE/THF (1:2) to afford 2-amino-3-[6-(3-methyl-2-oxo-1,3-benzoxazol-5-yl)-1-benzofuran-2-yl]propanenitrile (250 mg, 90% purity) as an off-white solid. LCMS (ES, m/z): [M+H]⁺: 334.

### Synthesis of tert-butyl (2S)-2-({1-cyano-2-[6-(3-methyl-2-oxo-1,3-benzoxazol-5-yl)-1-benzofuran-2-yl]ethyl}carbamoyl)-1,4-oxazepane-4-carboxylate

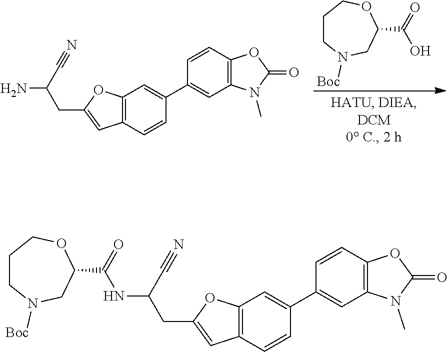 [SMILES: Cn1c(=O)oc2ccc(-c3ccc4cc(CC(N)C#N)oc4c3)cc21 ; CC(C)(C)OC(=O)N1CCCO[C@H](C(=O)O)C1 ; Cn1c(=O)oc2ccc(-c3ccc4cc(CC(C#N)NC(=O)[C@@H]5CN(C(=O)OC(C)(C)C)CCCO5)oc4c3)cc21]

To a stirred mixture of 2-amino-3-[6-(3-methyl-2-oxo-1,3-benzoxazol-5-yl)-1-benzofuran-2-yl]propanenitrile (92 mg, 0.27 mmol, 1.05 equiv), (2S)-4-(tert-butoxycarbonyl)-1,4-oxazepane-2-carboxylic acid (65 mg, 0.265 mmol, 1.0 equiv) and DIEA (102 mg, 0.79 mmol, 3.0 equiv) in DCM (5 mL) were added HATU (120 mg, 0.31 mmol, 1.2 equiv) in portions at 0° C. The resulting mixture was stirred for additional 2 h at 0° C. Concentrated to remove the solvent, the residue was purified by silica gel column chromatography, eluted with PE/THF (1:1) to afford tert-butyl (2S)-2-({1-cyano-2-[6-(3-methyl-2-oxo-1,3-benzoxazol-5-yl)-1-benzofuran-2-yl]ethyl}carbamoyl)-1,4-oxazepane-4-carboxylate (130 mg, 90% purity) as an off-white semi-solid. LCMS (ES, m/z): [M+H]⁺: 561.

### Synthesis of (2S)—N-{1-cyano-2-[6-(3-methyl-2-oxo-1,3-benzoxazol-5-yl)-1-benzofuran-2-yl]ethyl}-1,4-oxazepane-2-carboxamide

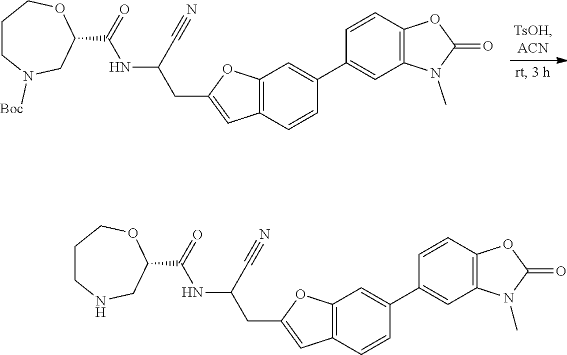 [SMILES: Cn1c(=O)oc2ccc(-c3ccc4cc(CC(C#N)NC(=O)[C@@H]5CN(C(=O)OC(C)(C)C)CCCO5)oc4c3)cc21 ; Cn1c(=O)oc2ccc(-c3ccc4cc(CC(C#N)NC(=O)[C@@H]5CNCCCO5)oc4c3)cc21]

Into a 25 mL round-bottom flask were added tert-butyl (2S)-2-({1-cyano-2-[6-(3-methyl-2-oxo-1,3-benzoxazol-5-yl)-1-benzofuran-2-yl]ethyl}carbamoyl)-1,4-oxazepane-4-carboxylate (130 mg, 0.23 mmol, 1.0 equiv), TsOH (119 mg, 0.69 mmol, 3.0 equiv) and acetonitrile (5 mL) at room temperature. The resulting mixture was stirred for additional 3 h at room temperature. The reaction solution was purified by reversed-phase flash chromatography with the following conditions: column, C₁₈ silica gel; mobile phase, MeCN in Water (0.1% NH₃·H₂O), 10% to 50% gradient in 12 min; detector, UV 254 nm. This resulted in (2S)—N-{1-cyano-2-[6-(3-methyl-2-oxo-1,3-benzoxazol-5-yl)-1-benzofuran-2-yl]ethyl}-1,4-oxazepane-2-carboxamide (20 mg, 18.7% yield) as a white solid.

### Analytical Data

LCMS (ES, m/z): [M+H]⁺: 461.

¹H NMR (400 MHz, DMSO-d₆): δ 8.75 (dd, J=8.4, 6.9 Hz, 1H), 7.90-7.87 (m, 1H), 7.72-7.64 (m, 2H), 7.61-7.58 (m, 1H), 7.51-7.47 (m, 1H), 7.42-7.40 (m, 1H), 6.83 (s, 1H), 5.25-5.14 (m, 1H), 4.06-3.96 (m, 1H), 3.94-3.85 (m, 1H), 3.79-3.69 (m, 1H), 3.54-3.38 (m, 5H), 3.18-3.06 (m, 1H), 2.84-2.57 (m, 3H), 1.81-1.66 (m, 2H).

### Example 25. Synthesis of compound: (2S)—N-{1-cyano-2-[5-(3-methyl-2-oxo-1,3-benzoxazol-5-yl)-1-benzofuran-2-yl]ethyl}-1,4-oxazepane-2-carboxamide

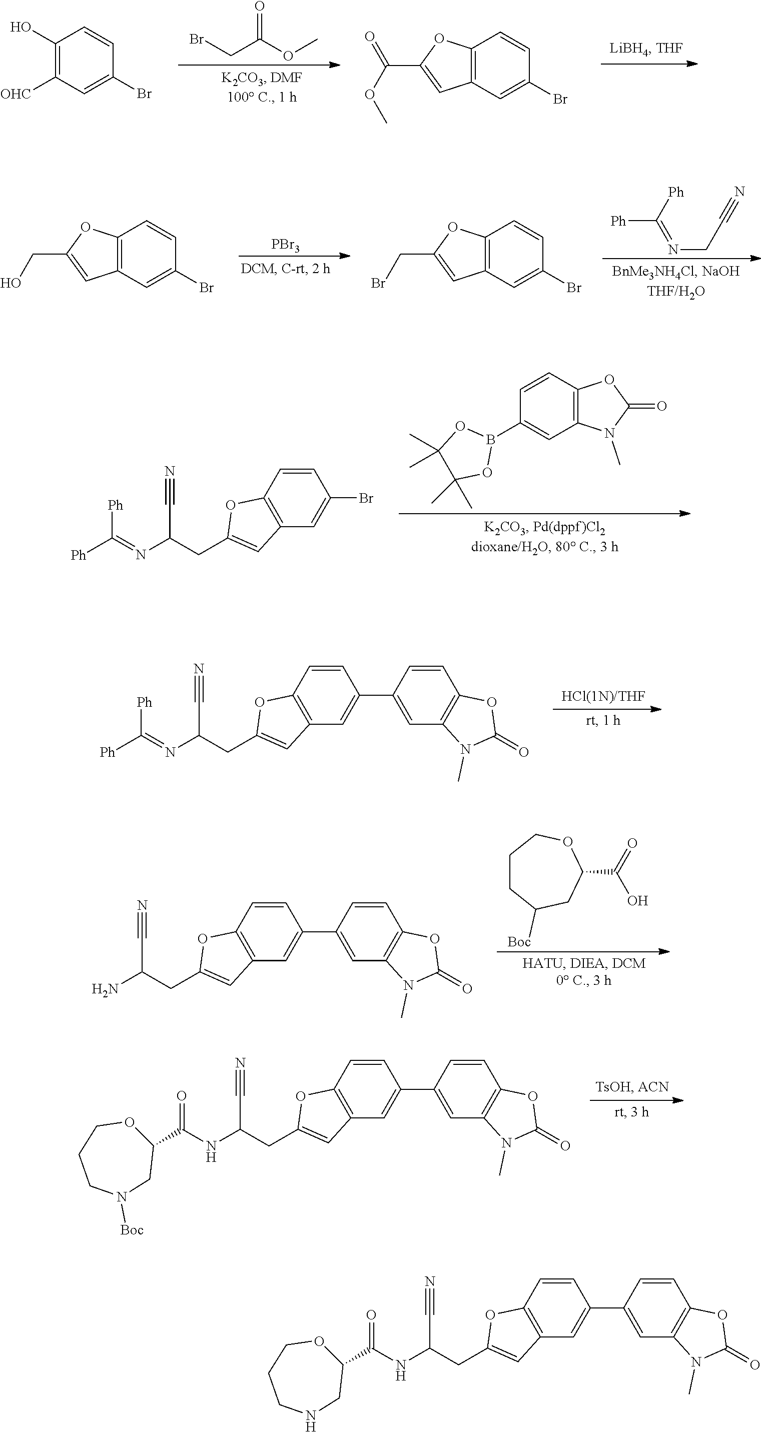

### Synthesis of methyl 5-bromo-1-benzofuran-2-carboxylate

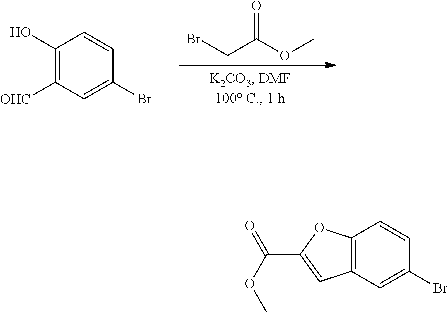

To a stirred solution of 5-bromo-2-hydroxybenzaldehyde (10 g, 49.747 mmol, 1.0 equiv) and K₂CO₃ (20.63 g, 149.241 mmol, 3.0 equiv) in DMF (200 mL) was added methyl 2-bromoacetate (7.61 g, 49.747 mmol, 1.0 equiv) under nitrogen atmosphere. The resulting mixture was stirred for 1 h at 100° C. under nitrogen atmosphere. The resulting mixture was extracted with EtOAc (3×400 mL). The combined organic layers were washed with brine (3×400 mL), dried over anhydrous Na₂SO₄. After filtration, the filtrate was concentrated under reduced pressure. The crude product was re-crystallized from PE/ethyl acetate (10:1 50 mL) to afford methyl 5-bromo-1-benzofuran-2-carboxylate (8.4 g) as an off-white solid. ¹H NMR (400 MHz, Chloroform-d) δ 7.85 (d, J=2.0 Hz, 1H), 7.57 (dd, J=9.0, 2.0 Hz, 1H), 7.49 (d, J=7.7 Hz, 2H), 4.01 (s, 3H).

### Synthesis of (5-bromo-1-benzofuran-2-yl)methanol

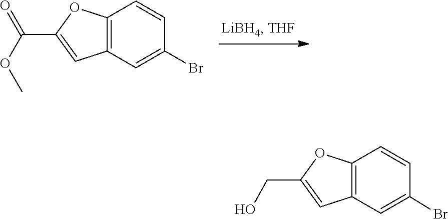

To a stirred solution of methyl 5-bromo-1-benzofuran-2-carboxylate (4 g, 15.682 mmol, 1 equiv) in THE (40 mL) was added lithium borohydride (2.0M in THF) (23.52 mL, 47.046 mmol, 3 equiv)) dropwise at 0° C. under nitrogen atmosphere. The resulting mixture was stirred for 8 h at room temperature under nitrogen atmosphere. The resulting mixture was extracted with EtOAc (3×50 mL). The combined organic layers were dried over anhydrous Na₂SO₄. After filtration, the filtrate was concentrated under reduced pressure. After filtration, the filtrate was concentrated under reduced pressure. The residue was purified by silica gel column chromatography, eluted with PE/EA (3:1) to afford (5-bromo-1-benzofuran-2-yl)methanol (3.3 g) as a light yellow oil. ¹H NMR (400 MHz, DMSO-d₆) δ 7.82 (d, J=2.1 Hz, 1H), 7.53 (d, J=8.7 Hz, 1H), 7.41 (dd, J=8.7, 2.1 Hz, 1H), 6.76 (d, J=1.0 Hz, 1H), 5.53 (t, J=5.9 Hz, 1H), 4.58 (dd, J=6.0, 0.9 Hz, 2H).

### Synthesis of 5-bromo-2-(bromomethyl)-1-benzofuran

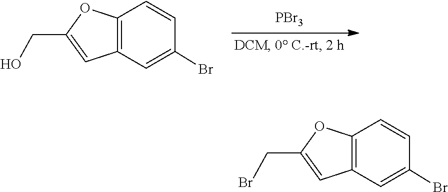 [SMILES: OCc1cc2cc(Br)ccc2o1 ; "PBr3" ; BrCc1cc2cc(Br)ccc2o1]

A solution of (5-bromo-1-benzofuran-2-yl)methanol (3.3 g, 14.534 mmol, 1 equiv) in DCM (40 mL) was followed by the addition of PBr₃ (1.97 g, 7.267 mmol, 0.5 equiv) at 0° C. The resulting mixture was stirred for additional 2 h at room temperature. The reaction was quenched by the addition of Water at 0° C. The resulting mixture was extracted with DCM (3×50 mL). The combined organic layers were washed with brine (1×50 mL), dried over anhydrous Na₂SO₄. After filtration, the filtrate was concentrated under reduced pressure. This resulted in 5-bromo-2-(bromomethyl)-1-benzofuran (3.6 g) as a yellow oil. The crude product mixture was used in the next step directly without further purification.

### Synthesis of 3-(5-bromo-1-benzofuran-2-yl)-2-[(diphenylmethylidene)amino]propanenitrile

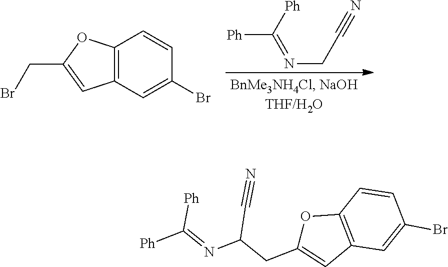 [SMILES: BrCc1cc2cc(Br)ccc2o1 ; N#CCN=C(c1ccccc1)c1ccccc1 ; N#CC(Cc1cc2cc(Br)ccc2o1)N=C(c1ccccc1)c1ccccc1]

A solution of 5-bromo-2-(bromomethyl)-1-benzofuran (3.6 g, 12.416 mmol, 1 equiv) in THE (40 mL) was added 2-[(diphenylmethylidene)amino]acetonitrile (2.73 g, 12.416 mmol, 1.0 equiv), benzyltrimethylazanium chloride (0.23 g, 1.242 mmol, 0.1 equiv), NaOH (0.99 g, 24.832 mmol, 2 equiv) in H₂O (4 mL) was stirred for 13 h at room temperature. The resulting mixture was extracted with EtOAc (3×50 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EA (20:1) to afford 3-(5-bromo-1-benzofuran-2-yl)-2-[(diphenylmethylidene)amino]propanenitrile (4.1 g, 90% purity) as a yellow oil. LCMS (ES, m/z): [M+H]⁺:429.

### 5. Synthesis of 2-[(diphenylmethylidene)amino]-3-[5-(3-methyl-2-oxo-1,3-benzoxazol-5-yl)-1-benzofuran-2-yl]propanenitrile

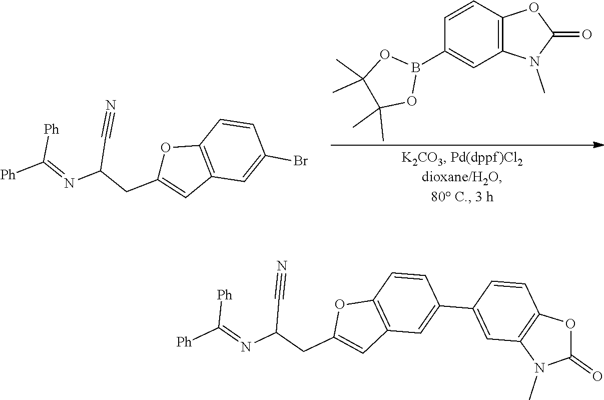 [SMILES: N#CC(Cc1cc2cc(Br)ccc2o1)N=C(c1ccccc1)c1ccccc1 ; Cn1c(=O)oc2ccc(B3OC(C)(C)C(C)(C)O3)cc21 ; Cn1c(=O)oc2ccc(-c3ccc4oc(CC(C#N)N=C(c5ccccc5)c5ccccc5)cc4c3)cc21]

A solution of 3-(5-bromo-1-benzofuran-2-yl)-2-[(diphenylmethylidene)amino]propanenitrile (1 g, 2.329 mmol, 1.0 equiv), 3-methyl-5-(4,4,5,5-tetramethyl-1,3,2-dioxaborolan-2-yl)-1,3-benzoxazol-2-one (0.7 g, 2.562 mmol, 1.1 equiv), K₂CO₃ (643.84 mg, 4.658 mmol, 2 equiv) and Pd(dppf)Cl₂CH₂Cl₂ (190 mg, 0.233 mmol, 0.1 equiv) in 1,4-dioxane (15 mL) H₂O (1.5 mL) was stirred for 2 h at 80° C. under nitrogen atmosphere. The residue was purified by silica gel column chromatography, eluted with PE/EA (2:1) to afford 2-[(diphenylmethylidene)amino]-3-[5-(3-methyl-2-oxo-1,3-benzoxazol-5-yl)-1-benzofuran-2-yl]propanenitrile (1.0 g 86.29%) as a white semi-solid. LCMS (ES, m/z): [M+H]⁺:498.

### Synthesis of 2-amino-3-[5-(3-methyl-2-oxo-1,3-benzoxazol-5-yl)-1-benzofuran-2-yl]propanenitrile

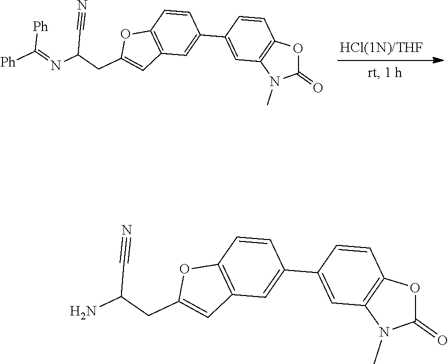 [SMILES: Cn1c(=O)oc2ccc(-c3ccc4oc(CC(C#N)N=C(c5ccccc5)c5ccccc5)cc4c3)cc21 ; Cn1c(=O)oc2ccc(-c3ccc4oc(CC(N)C#N)cc4c3)cc21]

Into a 100 mL round-bottom flask were added 2-[(diphenylmethylidene)amino]-3-[5-(3-methyl-2-oxo-1,3-benzoxazol-5-yl)-1-benzofuran-2-yl]propanenitrile (500 mg, 1.005 mmol, 1 equiv), THF (25 mL), H₂O (2.5 mL) and HCl (1M) (1 mL) at room temperature. The resulting mixture was stirred for additional 3 h at room temperature. The mixture was basified to pH 12 with NaOH. The resulting mixture was extracted with EtOAc (3×50 mL). The combined organic layers were washed with brine (3×30 mL), dried over anhydrous Na₂SO₄. After filtration, the filtrate was concentrated under reduced pressure. The residue was purified by silica gel column chromatography, eluted with PE/THF (1:2) to afford 2-amino-3-[5-(3-methyl-2-oxo-1,3-benzoxazol-5-yl)-1-benzofuran-2-yl]propanenitrile (240 mg, 90% purity) as a off-white solid. LCMS (ES, m/z): [M+H]⁺:334.

### Synthesis of tert-butyl (2S)-2-({1-cyano-2-[5-(3-methyl-2-oxo-1,3-benzoxazol-5-yl)-1-benzofuran-2-yl]ethyl}carbamoyl)-1,4-oxazepane-4-carboxylate

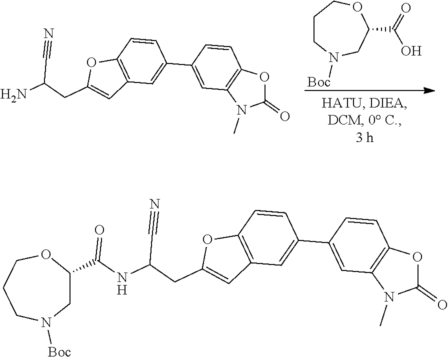 [SMILES: Cn1c(=O)oc2ccc(-c3ccc4oc(CC(N)C#N)cc4c3)cc21 ; CC(C)(C)OC(=O)N1CCCO[C@H](C(=O)O)C1 ; Cn1c(=O)oc2ccc(-c3ccc4oc(CC(C#N)NC(=O)[C@@H]5CN(C(=O)OC(C)(C)C)CCCO5)cc4c3)cc21]

To a stirred mixture of 2-amino-3-[5-(3-methyl-2-oxo-1,3-benzoxazol-5-yl)-1-benzofuran-2-yl]propanenitrile (85 mg, 0.255 mmol, 1.0 equiv), (2S)-4-(tert-butoxycarbonyl)-1,4-oxazepane-2-carboxylic acid (62 mg, 0.255 mmol, 1.0 equiv) and DIEA (98 mg, 0.765 mmol, 3 equiv) in DCM (5 mL) were added HATU (116 mg, 0.306 mmol, 1.2 equiv) in portions at 0° C. The resulting mixture was stirred for additional 3 h at 0° C. The residue was purified by silica gel column chromatography, eluted with PE/THF (1:1) to afford tert-butyl (2S)-2-({1-cyano-2-[5-(3-methyl-2-oxo-1,3-benzoxazol-5-yl)-1-benzofuran-2-yl]ethyl}carbamoyl)-1,4-oxazepane-4-carboxylate (140 mg, 90% purity) as a off-white semi-solid. LCMS (ES, m/z): [M+H]⁺:561.

### Synthesis of (2S)—N-{1-cyano-2-[5-(3-methyl-2-oxo-1,3-benzoxazol-5-yl)-1-benzofuran-2-yl]ethyl}-1,4-oxazepane-2-carboxamide

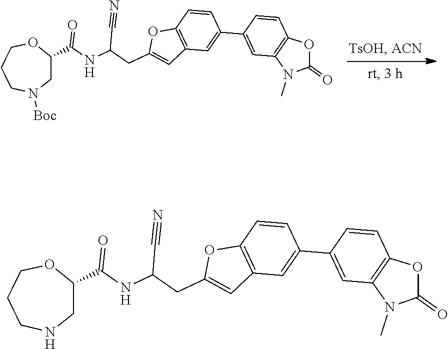 [SMILES: Cn1c(=O)oc2ccc(-c3ccc4oc(CC(C#N)NC(=O)[C@@H]5CN(C(=O)OC(C)(C)C)CCCO5)cc4c3)cc21 ; Cn1c(=O)oc2ccc(-c3ccc4oc(CC(C#N)NC(=O)[C@@H]5CNCCCO5)cc4c3)cc21]

Into a 50 mL round-bottom flask were added tert-butyl (2S)-2-({1-cyano-2-[5-(3-methyl-2-oxo-1,3-benzoxazol-5-yl)-1-benzofuran-2-yl]ethyl}carbamoyl)-1,4-oxazepane-4-carboxylate (140 mg, 0.250 mmol, 1.0 equiv) in acetonitrile (4 mL) and TsOH (129 mg, 0.749 mmol, 3.00 equiv) at room temperature. The resulting mixture was stirred at room temperature for additional 3 h. The residue was purified by reversed-phase flash chromatography with the following conditions: column, C₁₈ silica gel; mobile phase, MeCN in Water (0.1% NH₃·H₂O), 0% to 45% gradient in 15 min; detector, UV 254 nm. This resulted in (2S)—N-{1-cyano-2-[5-(3-methyl-2-oxo-1,3-benzoxazol-5-yl)-1-benzofuran-2-yl]ethyl}-1,4-oxazepane-2-carboxamide (20.5 mg, 17.83% yield, 97% purity) as a white solid.

### Analytical Data

LCMS (ES, m/z): [M+H]⁺:461.2

¹H NMR (400 MHz, DMSO-d₆) δ 8.75 (t, J=8.8 Hz, 1H), 7.90 (d, J=2.0 Hz, 1H), 7.67-7.56 (m, 3H), 7.47-7.37 (m, 2H), 6.85 (s, 1H), 5.20 (dq, J=15.4, 8.0 Hz, 1H), 4.01 (ddd, J=24.0, 7.9, 3.6 Hz, 1H), 3.95-3.83 (m, 1H), 3.73 (dtd, J=12.0, 7.5, 4.0 Hz, 1H), 3.54-3.42 (m, 5H), 3.12 (ddd, J=29.4, 14.3, 3.7 Hz, 1H), 2.86-2.57 (m, 3H), 1.75 (dd, J=14.5, 8.8 Hz, 2H).

### Example 26. Synthesis of compound 118b: (2S)—N-(1-cyano-2-(5-(3-methyl-2-oxo-2,3-dihydrobenzo[d]oxazol-5-yl)thieno[3,2-b]thiophen-2-yl)ethyl)-1,4-oxazepane-2-carboxamide

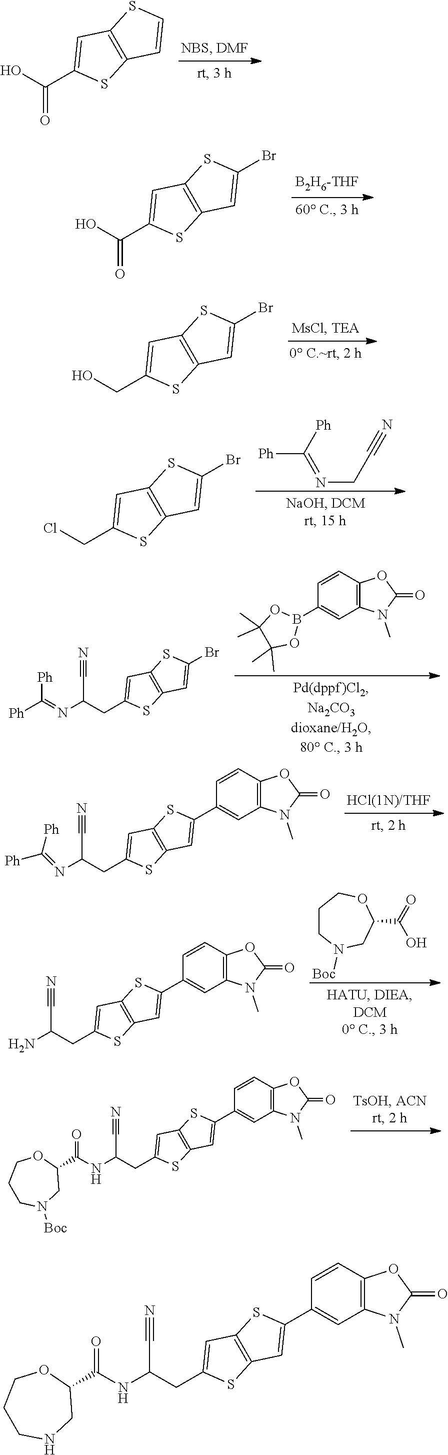

### Synthesis of 5-bromothieno[3,2-b]thiophene-2-carboxylic acid

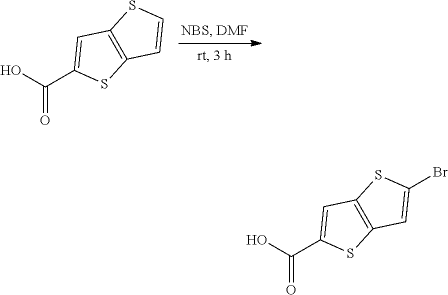 [SMILES: O=C(O)c1cc2sccc2s1 ; O=C(O)c1cc2sc(Br)cc2s1]

To a solution of thieno[3,2-b]thiophene-2-carboxylic acid (2.0 g, 10.86 mmol, 1.0 equiv) in DMF (20 mL), NBS (2.13 g, 11.94 mmol, 1.1 equiv) was added at 0° C. The mixture was stirred for 3 h at room temperature. Concentrated to remove the solvent, the residue was purified by silica gel column chromatography, eluted with PE/EA (5:1) to afford 5-bromothieno[3,2-b]thiophene-2-carboxylic acid (2.1 g, 73.5%) as yellow green solid. LCMS (ES, m/z): [M−H]—: 261 (negative signal).

### Synthesis of {5-bromothieno[3,2-b]thiophen-2-yl}methanol

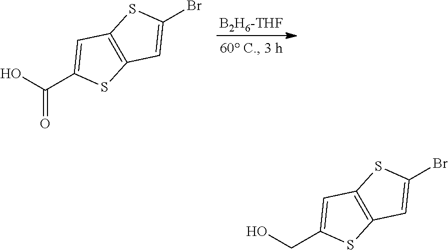 [SMILES: "B2H6-THF" ; O=C(O)c1cc2sc(Br)cc2s1 ; OCc1cc2sc(Br)cc2s1]

A solution of 5-bromothieno[3,2-b]thiophene-2-carboxylic acid (2 g, 7.60 mmol, 1.0 equiv) in THF (100 mL) was treated with oxolane borane (1 M in THF) (22.8 mL, 22.80 mmol, 3.0 equiv) for 20 min at room temperature under nitrogen atmosphere. The mixture was heated to 60° C. and stirred for 3 h under nitrogen atmosphere. The mixture was allowed to cool down to room temperature. The reaction was quenched by the addition of H₂O (80 ml), extracted with EtOAc (3×20 mL). The combined organic layer was washed with brine (2×30 mL), dried over anhydrous Na₂SO₄. After filtration, the filtrate was concentrated under reduced pressure. The residue was purified by silica gel column chromatography, eluted with PE/THF (4:1) to afford {5-bromothieno[3,2-b]thiophen-2-yl}methanol (948 mg, 50%) as a yellow green oil. LCMS (ES, m/z): [M−H₂O+H]⁺: 231.

### Synthesis of 2-bromo-5-(chloromethyl)thieno[3,2-b]thiophene

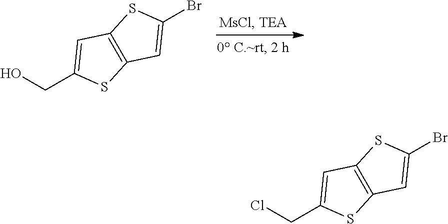 [SMILES: OCc1cc2sc(Br)cc2s1 ; ClCc1cc2sc(Br)cc2s1]

To a mixture of {5-bromothieno[3,2-b]thiophen-2-yl}methanol (948 mg, 3.80 mmol, 1.0 equiv), DIEA (984 mg, 7.61 mmol, 2.0 equiv) in DCM (10 mL), MsCl (523 mg, 4.56 mmol, 1.2 equiv) was added dropwise at 0° C. After addition, the reaction was stirred for 2 h at room temperature. The reaction was quenched with water (20 mL), extracted with dichloromethane (20 mL×1), dried over anhydrous sodium sulfate. After filtration, the filtrate was concentrated under reduced pressure. This result in 2-bromo-5-(chloromethyl)thieno[3,2-b]thiophene (900 mg, 88.4%) as colorless oil and used to the next step without further purification. (no MS signal in LCMS).

### Synthesis of 3-{5-bromothieno[3,2-b]thiophen-2-yl}-2-[(diphenylmethylidene)amino]propanenitrile

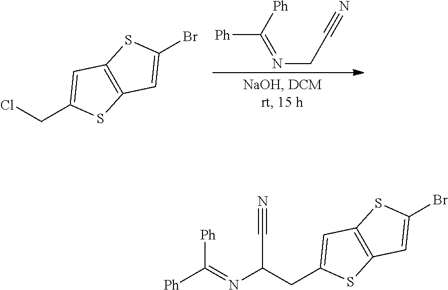

To a solution of 2-bromo-5-(chloromethyl)thieno[3,2-b]thiophene (900 mg, 3.36 mmol, 1.0 equiv) and 2-[(diphenylmethylidene)amino]acetonitrile (815 mg, 3.70 mmol, 1.1 equiv) in DCM (10 mL), benzyltrimethylazanium chloride (65 mg, 0.35 mmol, 0.10 equiv) was added room temperature. This was followed by the addition of NaOH (269 mg, 6.73 mmol, 2.0 equiv) in H₂O (1 mL) at room temperature. The mixture was stirred for 15 h. The reaction was diluted with water (20 mL), extracted with dichloromethane (20 mL×1), dried over anhydrous sodium sulfate. After filtration, the filtrate was concentrated under reduced pressure, the residue was purified by silica gel column with ethyl acetate/petroleum ether (5%). This result in 3-{5-bromothieno[3,2-b]thiophen-2-yl}-2-[(diphenylmethylidene)amino]propanenitrile (632 mg, 42%) as yellow oil and used to the next step without further purification. LCMS (ES, m/z): [M+H]⁺: 451.

### Synthesis of 2-[(diphenylmethylidene)amino]-3-[5-(3-methyl-2-oxo-1,3-benzoxazol-5-yl)thieno[3,2-b]thiophen-2-yl]propanenitrile

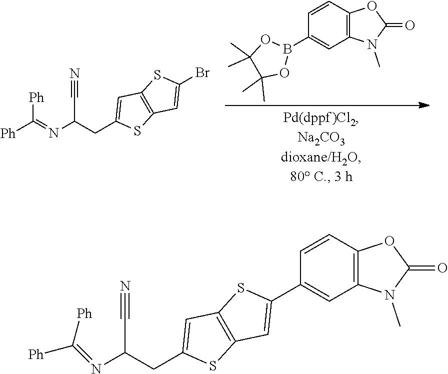

To a 100 mL round bottom flask were added 3-{5-bromothieno[3,2-b]thiophen-2-yl}-2-[(diphenylmethylidene)amino]propanenitrile (580 mg, 1.28 mmol, 1.0 equiv), 3-methyl-5-(4,4,5,5-tetramethyl-1,3,2-dioxaborolan-2-yl)-1,3-benzoxazol-2-one (424 mg, 1.54 mmol, 1.2 equiv), Na₂CO₃ (272 mg, 2.57 mmol, 2.0 equiv), 1,4-dioxane (10 mL), H₂O (1 mL) in sequence. After insert nitrogen gas, Pd(dppf)Cl₂ (105 mg, 0.13 mmol, 0.1 equiv) was added. The mixture was heated to 80° C. and stirred for 3 h. The reaction was cooled to room temperature, diluted with water (20 mL), extracted with dichloromethane (20 mL×2). The combined organic phase was dried over anhydrous sodium sulfate. After filtration, the filtrate was concentrated under reduced pressure, the residue was purified by silica gel column with ethyl acetate/petroleum ether (35%). The fraction was concentrated, this result in 2-[(diphenylmethylidene)amino]-3-[5-(3-methyl-2-oxo-1,3-benzoxazol-5-yl)thieno[3,2-b]thiophen-2-yl]propanenitrile (420 mg, 63%) as light yellow solid. LCMS (ES, m/z): [M+H]⁺: 520.

### Synthesis of tert-butyl (2S)-2-((1-cyano-2-(5-(3-methyl-2-oxo-2,3-dihydrobenzo[d]oxazol-5-yl)thieno[3,2-b]thiophen-2-yl)ethyl)carbamoyl)-1,4-oxazepane-4-carboxylate

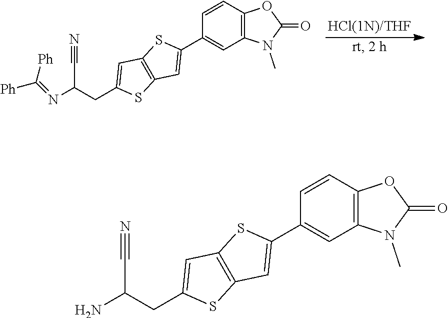 [SMILES: Cn1c(=O)oc2ccc(-c3cc4sc(CC(C#N)N=C(c5ccccc5)c5ccccc5)cc4s3)cc21 ; "HCl(1N)/THF" ; "rt, 2 h" ; Cn1c(=O)oc2ccc(-c3cc4sc(CC(N)C#N)cc4s3)cc21]

To a solution of 2-[(diphenylmethylidene)amino]-3-[5-(3-methyl-2-oxo-1,3-benzoxazol-5-yl)thieno[3,2-b]thiophen-2-yl]propanenitrile (250 mg, 0.48 mmol, 1.0 equiv) in THF (2.5 mL), HCl (0.1 mL, 1 N in water) was added at room temperature. The reaction was stirred for 2 h, before diluted with water (10 mL), extracted with ethyl acetate (10 mL×2). The PH value of the aqueous phase was adjusted to 8-9 with K₂CO₃ solid, extracted with ethyl acetate (30 mL×3), dried over anhydrous sodium sulfate. After filtration, the filtrate was concentrated, the residue was purified by silica gel column with ethyl acetate/petroleum ether (10% to 100%). This result in 2-amino-3-[5-(3-methyl-2-oxo-1,3-benzoxazol-5-yl)thieno[3,2-b]thiophen-2-yl]propanenitrile (122 mg, 71%) as light yellow solid. LCMS (ES, m/z): [M+H]⁺: 356.

### Synthesis of tert-butyl 2-(2-amino-2-cyanoethyl)-5-(3-methyl-2-oxo-2,3-dihydrobenzo[d]oxazol-5-yl)-1H-pyrrole-1-carboxylate

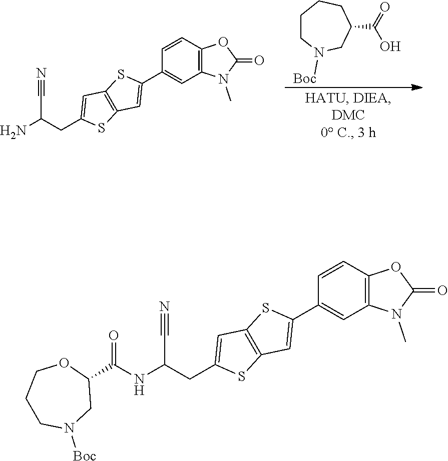 [SMILES: Cn1c(=O)oc2ccc(-c3cc4sc(CC(N)C#N)cc4s3)cc21 ; CC(C)(C)OC(=O)N1CCCC[C@H](C(=O)O)C1 ; Cn1c(=O)oc2ccc(-c3cc4sc(CC(C#N)NC(=O)[C@@H]5CN(C(=O)OC(C)(C)C)CCCO5)cc4s3)cc21]

To a solution of 2-amino-3-[5-(3-methyl-2-oxo-1,3-benzoxazol-5-yl)thieno[3,2-b]thiophen-2-yl]propanenitrile (122 mg, 0.34 mmol, 1.2 equiv) in DCM (2.5 mL) was treated with (2S)-4-(tert-butoxycarbonyl)-1,4-oxazepane-2-carboxylic acid (70 mg, 0.28 mmol, 1.0 equiv), DIEA (74 mg, 0.57 mmol, 2.0 equiv). This was followed by the addition of HATU (130 mg, 0.34 mmol, 1.2 equiv) in portions at 0° C. The resulting mixture was stirred for 3 h at 0° C. Concentrated to remove the solvent, the residue was purified by silica gel column chromatography, eluted with PE/THF (1:1) to afford tert-butyl (2S)-2-{[(1S)-1-cyano-2-[5-(3-methyl-2-oxo-1,3-benzoxazol-5-yl)thieno[3,2-b]thiophen-2-yl]ethyl]carbamoyl}-1,4-oxazepane-4-carboxylate (130 mg, 78%) as white solid. LCMS (ES, m/z): [M+H]⁺: 583.

### Synthesis of (2S)—N-(1-cyano-2-(5-(3-methyl-2-oxo-2,3-dihydrobenzo[d]oxazol-5-yl)thieno[3,2-b]thiophen-2-yl)ethyl)-1,4-oxazepane-2-carboxamide

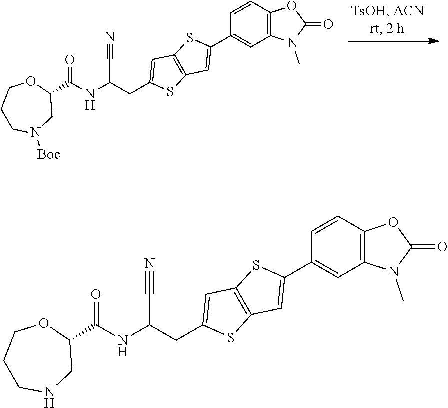 [SMILES: Cn1c(=O)oc2ccc(-c3cc4sc(CC(C#N)NC(=O)[C@@H]5CN(C(=O)OC(C)(C)C)CCCO5)cc4s3)cc21 ; Cn1c(=O)oc2ccc(-c3cc4sc(CC(C#N)NC(=O)[C@@H]5CNCCCO5)cc4s3)cc21]

Into a 25 mL round-bottom flask were added tert-butyl (2S)-2-{[(1S)-1-cyano-2-[5-(3-methyl-2-oxo-1,3-benzoxazol-5-yl)thieno[3,2-b]thiophen-2-yl]ethyl]carbamoyl}-1,4-oxazepane-4-carboxylate (130 mg, 0.22 mmol, 1.0 equiv), TsOH (115 mg, 0.67 mmol, 3.0 equiv) and ACN (2 mL) at room temperature. The resulting mixture was stirred for 3 h at room temperature. The reaction solution was purified by reversed-phase flash chromatography with the following conditions: column, C₁₈ silica gel-120 g; mobile phase, MeCN in Water (0.1% NH₃·H₂O), 10% to 50% gradient in 10 min; detector, UV 254 nm. The fraction of the target was freezing dried, this resulted in (2S)—N-[(1S)-1-cyano-2-[5-(3-methyl-2-oxo-1,3-benzoxazol-5-yl)thieno[3,2-b]thiophen-2-yl]ethyl]-1,4-oxazepane-2-carboxamide (46 mg, 42.7%) as white solid.

### Analytical Data

LCMS (ES, m/z): [M+H]⁺: 483.4

¹H NMR (400 MHz, DMSO-d₆) δ 8.71 (dd, J=8.5, 3.0 Hz, 1H), 7.84 (s, 1H), 7.60 (s, 1H), 7.49-7.32 (m, 3H), 5.12-4.93 (m, 1H), 4.08-3.82 (m, 2H), 3.82-3.66 (m, 1H), 3.59-3.44 (m, 2H), 3.40 (s, 3H), 3.18-3.04 (m, 1H), 2.87-2.53 (m, 3H), 1.82-1.67 (m, 2H).

### Example 27. Synthesis of compound 129b: (2S)—N-(1-cyano-2-(5-(3-methyl-2-oxo-2,3-dihydrobenzo[d]oxazol-5-yl)-1H-indol-2-yl)ethyl)-1,4-oxazepane-2-carboxamide

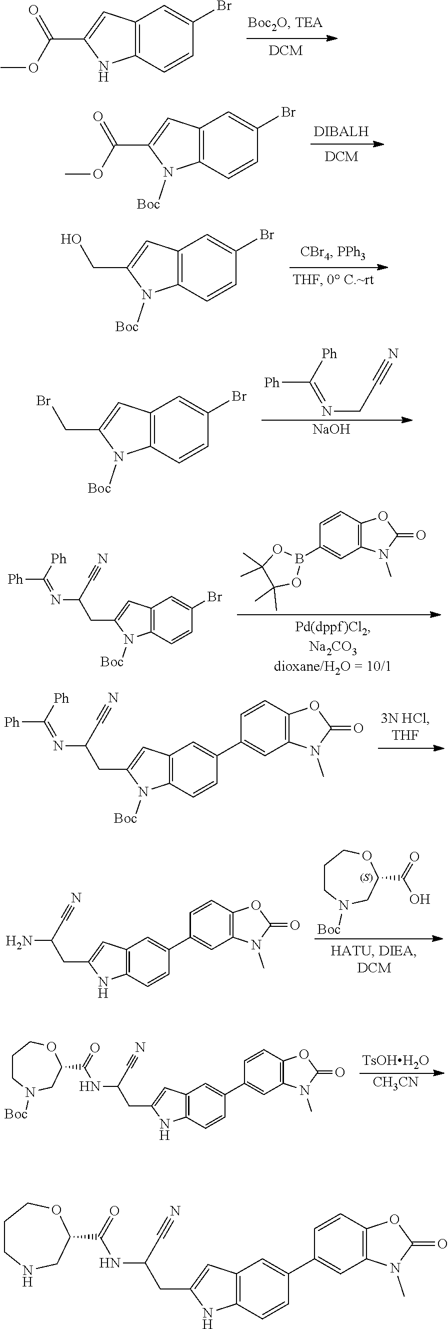

### Synthesis of 1-(tert-butyl) 2-methyl 5-bromo-1H-indole-1,2-dicarboxylate

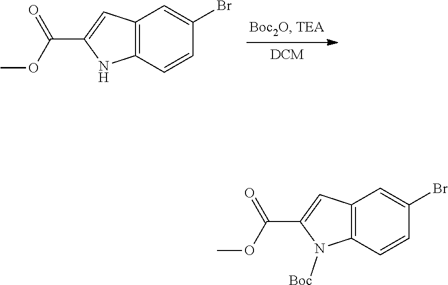 [SMILES: COC(=O)c1cc2cc(Br)ccc2[nH]1 ; COC(=O)c1cc2cc(Br)ccc2n1C(=O)OC(C)(C)C]

A solution of methyl 5-bromo-1H-indole-2-carboxylate (5 g, 19.68 mmol, 1.0 equiv) in DCM (70 mL) was treated with DMAP (0.24 g, 1.97 mmol, 0.1 equiv) at room temperature. This was followed by the addition of di-tert-butyl dicarbonate (6.44 g, 29.52 mmol, 1.5 equiv) in portions at 0° C. The resulting mixture was stirred at room temperature for 12 h. The reaction was quenched by the addition of water (100 mL). The aqueous layer was extracted with CH₂Cl₂ (50 mL×1). The combined organic phase was dried over anhydrous sodium sulfate. After filtration, the filtrate was concentrated under reduced pressure, the residue was purified by silica gel column chromatography, eluted with PE/EA (10:1) to afford 1-tert-butyl 2-methyl 5-bromoindole-1,2-dicarboxylate (6 g, 86%) as a yellow oil. LCMS (ES) [M+1]⁺ m/z: 354.

### Synthesis of tert-butyl 5-bromo-2-(hydroxymethyl)-1H-indole-1-carboxylate

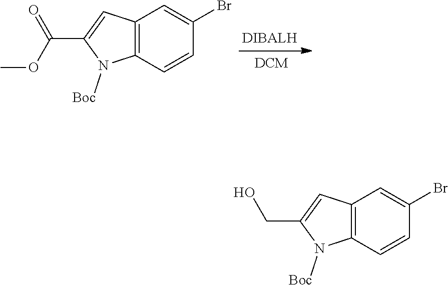 [SMILES: COC(=O)c1cc2cc(Br)ccc2n1C(=O)OC(C)(C)C ; CC(C)(C)OC(=O)n1c(CO)cc2cc(Br)ccc21]

Into a 250 mL 3-necked round-bottom flask were added 1-tert-butyl 2-methyl 5-bromoindole-1,2-dicarboxylate (5.5 g, 15.53 mmol, 1.0 equiv) and DCM (60 mL) at room temperature. To the above mixture was added DIBAL-H (1 M in hexane) (38.8 mL, 38.82 mmol, 2.5 equiv) dropwise at −78° C. The resulting mixture was stirred at −78° C. for 2 h. The reaction was quenched by the addition of water (20 mL) at −60° C. The resulting mixture was filtered, the filter cake was washed with DCM (30 mL×2). The filtrate was concentrated under reduced pressure. The residue was purified by silica gel column chromatography, eluted with PE/EA (5:1) to afford tert-butyl 5-bromo-2-(hydroxymethyl)indole-1-carboxylate (2.2 g, 43%) as a yellow oil. LCMS (ES) [M+1]⁺ m/z: 326.

### Synthesis of tert-butyl 5-bromo-2-(bromomethyl)-1H-indole-1-carboxylate

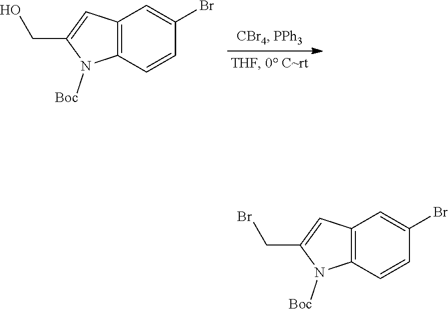 [SMILES: CC(C)(C)OC(=O)n1c(CO)cc2cc(Br)ccc21 ; CC(C)(C)OC(=O)n1c(CBr)cc2cc(Br)ccc21]

To a solution of tert-butyl 5-bromo-2-(hydroxymethyl)indole-1-carboxylate (500 mg, 1.53 mmol, 1.0 equiv) and PPh₃ (804 mg, 3.06 mmol, 2.0 equiv) in THF (5 mL), was added CBr₄ (762 mg, 2.30 mmol, 1.5 equiv) in portions at 0° C. The resulting mixture was stirred at room temperature for 2 h. The resulting mixture was concentrated under vacuum. The crude product was used in the next step directly without further purification.

### Synthesis of tert-butyl 5-bromo-2-(2-cyano-2-((diphenylmethylene)amino)ethyl)-1H-indole-1-carboxylate

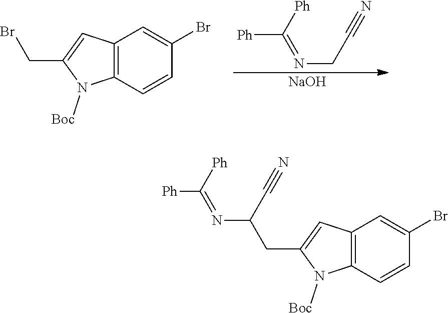

To a solution of tert-butyl 5-bromo-2-(bromomethyl)indole-1-carboxylate (540 mg, 1.39 mmol, 1.0 equiv), benzyl(chloro)trimethylazanium (26 mg, 0.14 mmol, 0.1 equiv) in THF (10 mL) was treated with NaOH (111 mg, 2.77 mmol, 2.0 equiv) at room temperature. The resulting mixture was stirred at room temperature for 5 h. The reaction was quenched by the addition of water (20 mL). The aqueous layer was extracted with EtOAc (40 mL×2), washed with brine (30 mL×1), dried over anhydrous sodium sulfate. After filtered and the filtrate was concentrated under reduced pressure. The residue was purified by silica gel column chromatography, eluted with PE/EA (10:1) to afford tert-butyl 5-bromo-2-{2-cyano-2-[(diphenylmethylidene)amino]ethyl}indole-1-carboxylate (400 mg, 54.5%) as a yellow oil.

LCMS (ES) [M+1]⁺ m/z: 528.

### Synthesis of tert-butyl 2-(2-cyano-2-((diphenylmethylene)amino)ethyl)-5-(3-methyl-2-oxo-2,3-dihydrobenzo[d]oxazol-5-yl)-1H-indole-1-carboxylate

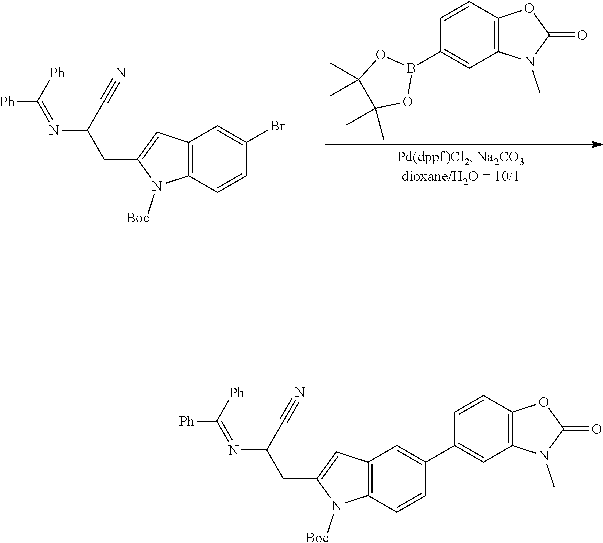 [SMILES: CC(C)(C)OC(=O)n1c(CC(C#N)N=C(c2ccccc2)c2ccccc2)cc2cc(Br)ccc21 ; Cn1c(=O)oc2ccc(B3OC(C)(C)C(C)(C)O3)cc21 ; Cn1c(=O)oc2ccc(-c3ccc4c(c3)cc(CC(C#N)N=C(c3ccccc3)c3ccccc3)n4C(=O)OC(C)(C)C)cc21]

To a solution of tert-butyl 5-bromo-2-{2-cyano-2-[(diphenylmethylidene)amino]ethyl}indole-1-carboxylate (400 mg, 0.76 mmol, 1.0 equiv), 3-methyl-5-(4,4,5,5-tetramethyl-1,3,2-dioxaborolan-2-yl)-1,3-benzoxazol-2-one (250 mg, 0.91 mmol, 1.2 equiv) in dioxane (5 mL) and H₂O (0.5 mL) were added Na₂CO₃ (160 mg, 1.51 mmol, 2.0 equiv) and Pd(dppf)Cl₂CH₂Cl₂ (62 mg, 0.076 mmol, 0.1 equiv) in sequence at room temperature. The resulting mixture was stirred at 80° C. for 2 h under nitrogen atmosphere. The reaction was cooled to room temperature, concentrated under reduced pressure. The residue was purified by silica gel column chromatography, eluted with PE/EA (1:1) to afford tert-butyl 2-{2-cyano-2-[(diphenylmethylidene)amino]ethyl}-5-(3-methyl-2-oxo-1,3-benzoxazol-5-yl)indole-1-carboxylate (300 mg, 66%) as a yellow solid. LCMS (ES) [M+1]⁺ m/z: 597.

### Synthesis of 2-amino-3-(5-(3-methyl-2-oxo-2,3-dihydrobenzo[d]oxazol-5-yl)-1H-indol-2-yl)propanenitrile

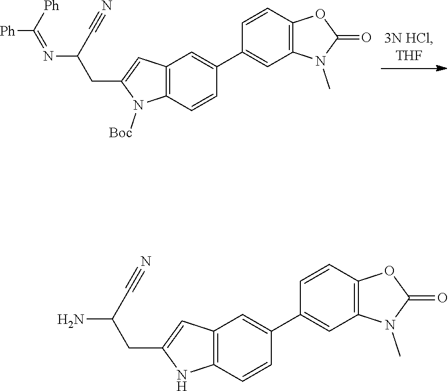 [SMILES: Cn1c(=O)oc2ccc(-c3ccc4c(c3)cc(CC(C#N)N=C(c3ccccc3)c3ccccc3)n4C(=O)OC(C)(C)C)cc21 ; Cn1c(=O)oc2ccc(-c3ccc4[nH]c(CC(N)C#N)cc4c3)cc21]

Into a 8 mL vial were added tert-butyl 2-{2-cyano-2-[(diphenylmethylidene)amino]ethyl}-5-(3-methyl-2-oxo-1,3-benzoxazol-5-yl)indole-1-carboxylate (300 mg, 0.50 mmol, 1.0 equiv), THF (3 mL) and 3 N HCl (1 mL) at room temperature. The resulting mixture was stirred at 60° C. for 12 h. The reaction was diluted with water (10 mL). The mixture was basified to pH 8 with saturated NaHCO₃ (aq.). The aqueous layer was extracted with EtOAc (20 mL×2). The combined organic phase was dried over anhydrous sodium sulfate. Filtered and the filtrate was concentrated under reduced pressure. The residue was purified by silica gel column chromatography, eluted with PE/EA (1:4) to afford 2-amino-3-[5-(3-methyl-2-oxo-1,3-benzoxazol-5-yl)-1H-indol-2-yl]propanenitrile (120 mg, 72%) as off-white solid. LCMS (ES) [M+1]⁺ m/z: 333.

### Synthesis of tert-butyl (2S)-2-((1-cyano-2-(5-(3-methyl-2-oxo-2,3-dihydrobenzo[d]oxazol-5-yl)-1H-indol-2-yl)ethyl)carbamoyl)-1,4-oxazepane-4-carboxylate

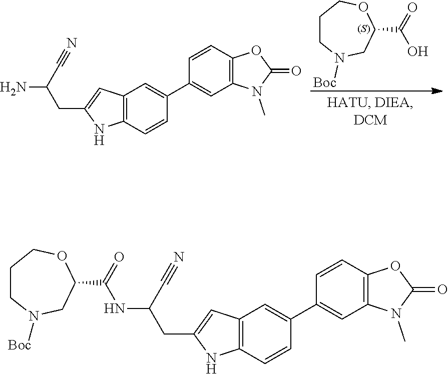

To a solution of 2-amino-3-[5-(3-methyl-2-oxo-1,3-benzoxazol-5-yl)-1H-indol-2-yl]propanenitrile (110 mg, 0.33 mmol, 1.0 equiv), (2S)-4-(tert-butoxycarbonyl)-1,4-oxazepane-2-carboxylic acid (90 mg, 0.36 mmol, 1.1 equiv), DIEA (128 mg, 0.99 mmol, 3.0 equiv) in DCM (3 mL) was treated with HATU (151 mg, 0.40 mmol, 1.2 equiv) at 0° C. The resulting mixture was stirred at 0° C. for 2 h. Concentrated to remove the solvent, the residue was purified by silica gel column chromatography, eluted with PE/EA (1:4) to afford tert-butyl (2S)-2-({1-cyano-2-[5-(3-methyl-2-oxo-1,3-benzoxazol-5-yl)-1H-indol-2-yl]ethyl}carbamoyl)-1,4-oxazepane-4-carboxylate (140 mg, 75.6%) as a yellow solid. LCMS (ES) [M+1]⁺ m/z: 560.

### Synthesis of (2S)—N-(1-cyano-2-(5-(3-methyl-2-oxo-2,3-dihydrobenzo[d]oxazol-5-yl)-1H-indol-2-yl)ethyl)-1,4-oxazepane-2-carboxamide

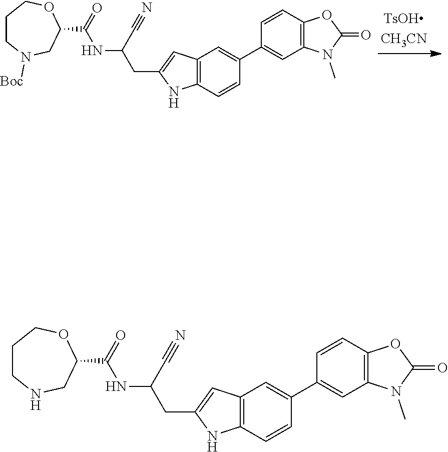 [SMILES: Cn1c(=O)oc2ccc(-c3ccc4[nH]c(CC(C#N)NC(=O)[C@@H]5CN(C(=O)OC(C)(C)C)CCCO5)cc4c3)cc21 ; Cn1c(=O)oc2ccc(-c3ccc4[nH]c(CC(C#N)NC(=O)[C@@H]5CNCCCO5)cc4c3)cc21]

Into a 8 mL vial were added tert-butyl (2S)-2-({1-cyano-2-[5-(3-methyl-2-oxo-1,3-benzoxazol-5-yl)-1H-indol-2-yl]ethyl}carbamoyl)-1,4-oxazepane-4-carboxylate (140 mg, 0.25 mmol, 1.0 equiv), acetonitrile (4 mL) and TSOH (129 mg, 0.75 mmol, 3.0 equiv) at room temperature. The resulting mixture was stirred at room temperature for 4 h. The reaction was purified by reverse phase flash with the following conditions: mobile phase, MeCN in Water (0.1% NH₃·H₂O), 10% to 70% gradient in 10 min; This resulted in (2S)—N-{1-cyano-2-[5-(3-methyl-2-oxo-1,3-benzoxazol-5-yl)-1H-indol-2-yl]ethyl}-1,4-oxazepane-2-carboxamide (16 mg, 13.9%) as a white solid.

LCMS (ES) [M+1]⁺ m/z: 560.2.

¹H NMR (400 MHz, DMSO-d₆) δ 11.14 (dd, J=8.5, 2.1 Hz, 1H), 8.71 (dd, J=8.3, 6.0 Hz, 1H), 7.77 (d, J=2.1 Hz, 1H), 7.55 (s, 1H), 7.44-7.33 (m, 4H), 6.37 (t, J=2.5 Hz, 1H), 5.24-5.09 (m, 1H), 4.01 (ddd, J=25.0, 7.8, 3.7 Hz, 1H), 3.89 (dddd, J=12.3, 10.2, 6.0, 4.4 Hz, 1H), 3.79-3.68 (m, 1H), 3.42 (s, 3H), 3.13 (ddd, J=24.4, 14.2, 3.7 Hz, 1H), 2.86-2.57 (m, 3H), 1.96-1.56 (m, 2H).

### Example 28. Synthesis of Compound 137a and 137b

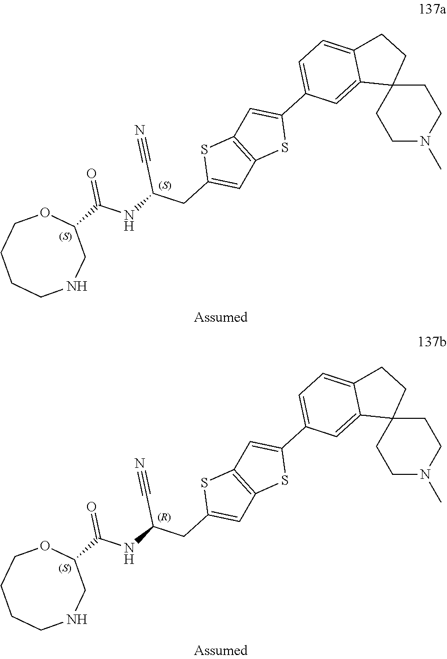

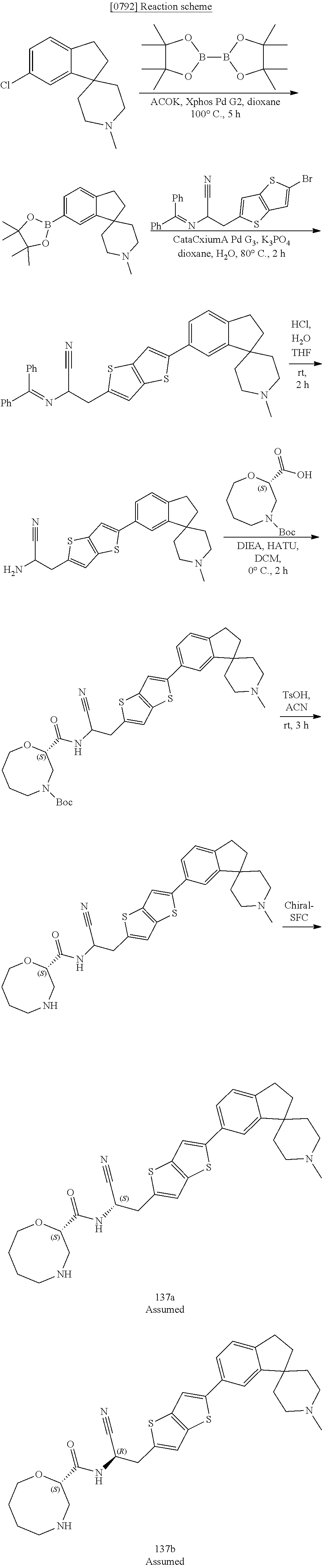

### Synthesis of 1′-methyl-6-(4,4,5,5-tetramethyl-1,3,2-dioxaborolan-2-yl)-2,3-dihydrospiro[indene-1,4′-piperidine]

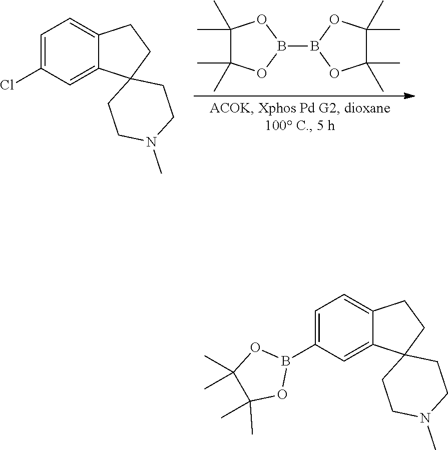

To a stirred solution of 6-chloro-1′-methyl-2,3-dihydrospiro[indene-1,4′-piperidine](400 mg, 1.697 mmol, 1.0 equiv) and bis(pinacolato)diboron (517 mg, 2.036 mmol, 1.2 equiv) in dioxane (6 mL) were added KOAc (333 mg, 3.394 mmol, 2.0 equiv) and 2nd Generation XPhos Precatalyst/(133 mg, 0.170 mmol, 0.1 equiv). The resulting mixture was stirred for 5 h at 100° C. under nitrogen atmosphere. The mixture was allowed to cool down to room temperature. The resulting mixture was filtered, the filter cake was washed with CH₂Cl₂ (3×10 mL). The filtrate was concentrated under reduced pressure. The crude product 1′-methyl-6-(4,4,5,5-tetramethyl-1,3,2-dioxaborolan-2-yl)-2,3-dihydrospiro[indene-1,4′-piperidine] (900 mg) was used in the next step directly without further purification. LCMS (ES) [M+1]⁺ m/z: 328.

### Synthesis of 2-[(diphenylmethylidene)amino]-3-(5-{1′-methyl-2,3-dihydrospiro[indene-1,4′-piperidin]-6-yl}thieno[3,2-b]thiophen-2-yl)propanenitrile

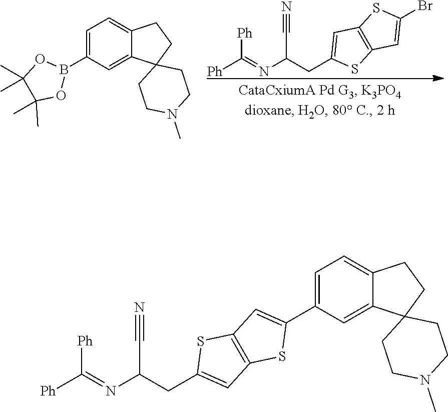 [SMILES: CN1CCC2(CCc3ccc(B4OC(C)(C)C(C)(C)O4)cc32)CC1 ; N#CC(Cc1cc2sc(Br)cc2s1)N=C(c1ccccc1)c1ccccc1 ; CN1CCC2(CCc3ccc(-c4cc5sc(CC(C#N)N=C(c6ccccc6)c6ccccc6)cc5s4)cc32)CC1]

To a stirred solution of 1′-methyl-6-(4,4,5,5-tetramethyl-1,3,2-dioxaborolan-2-yl)-2,3-dihydrospiro[indene-1,4′-piperidine](797 mg, 2.438 mmol, 2.2 equiv) and 3-{5-bromothieno[3,2-b]thiophen-2-yl}-2-[(diphenylmethylidene)amino]propanenitrile (500 mg, 1.108 mmol, 1.0 equiv) in dioxane (10 mL) and H₂O (1.2 mL) were added K₃PO₄ (470 mg, 2.216 mmol, 2.0 equiv) and cataCXium-A-Pd-G3 (80 mg, 0.111 mmol, 0.1 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (1:2) to afford 2-[(diphenylmethylidene)amino]-3-(5-{1′-methyl-2,3-dihydrospiro[indene-1,4′-piperidin]-6-yl}thieno[3,2-b]thiophen-2-yl)propanenitrile (400 mg, 63.1%) as a brown solid. LCMS (ES) [M+1]⁺ m/z: 572.

### Synthesis of 2-amino-3-(5-{1′-methyl-2,3-dihydrospiro[indene-1,4′-piperidin]-6-yl}thieno[3,2-b]thiophen-2-yl)propanenitrile

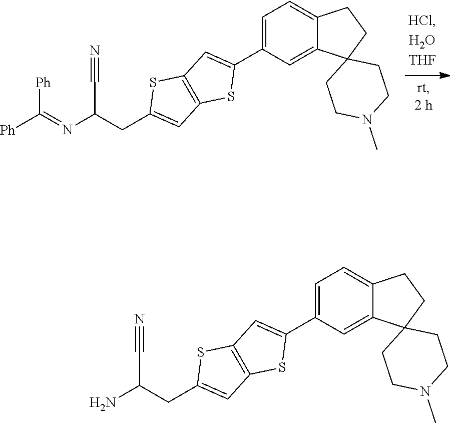 [SMILES: CN1CCC2(CCc3ccc(-c4cc5sc(CC(C#N)N=C(c6ccccc6)c6ccccc6)cc5s4)cc32)CC1 ; CN1CCC2(CCc3ccc(-c4cc5sc(CC(N)C#N)cc5s4)cc32)CC1]

To a stirred solution of 2-[(diphenylmethylidene)amino]-3-(5-{1′-methyl-2,3-dihydrospiro[indene-1,4′-piperidin]-6-yl}thieno[3,2-b]thiophen-2-yl)propanenitrile (400 mg, 0.700 mmol, 1.0 equiv) in THE (20 mL) and H₂O (2 mL) were added HCl (1M) (1 mL, 1.000 mmol). The resulting mixture was stirred for 2 h at room temperature. The resulting mixture was concentrated under reduced pressure. The resulting mixture was diluted with water (20 mL). The aqueous layer was extracted with EtOEt (2×20 mL). The aqueous layer was basified to pH 10 with NaOH (aq.) (1M). The aqueous layer was extracted with CH₂Cl₂ (5×20 mL). The combined organic layers were washed with brine (10 mL), dried over anhydrous Na₂SO₄. After filtration, the filtrate was concentrated under reduced pressure. This resulted in 2-amino-3-(5-{1′-methyl-2,3-dihydrospiro[indene-1,4′-piperidin]-6-yl}thieno[3,2-b]thiophen-2-yl)propanenitrile (220 mg, 77.1%) as a light yellow solid. LCMS (ES) [M+1]⁺ m/z: 408.

### Synthesis of tert-butyl (2S)-2-{[1-cyano-2-(5-{1′-methyl-2,3-dihydrospiro[indene-1,4′-piperidin]-6-yl}thieno[3,2-b]thiophen-2-yl)ethyl]carbamoyl}-1,4-oxazocane-4-carboxylate

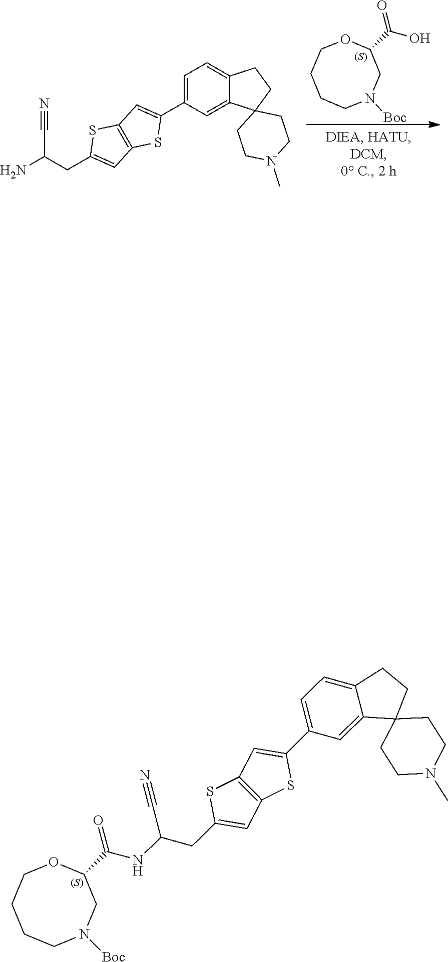

To a stirred solution of 2-amino-3-(5-{1′-methyl-2,3-dihydrospiro[indene-1,4′-piperidin]-6-yl}thieno[3,2-b]thiophen-2-yl)propanenitrile (172 mg, 0.425 mmol, 1.1 equiv) and (2S)-4-(tert-butoxycarbonyl)-1,4-oxazocane-2-carboxylic acid (100 mg, 0.386 mmol, 1.0 equiv) in DCM (2 mL) were added DIEA (149 mg, 1.158 mmol, 3.0 equiv) and HATU (175 mg, 0.463 mmol, 1.2 equiv) in portions at 0° C. under nitrogen atmosphere. The resulting mixture was stirred for 2 h at 0° C. under nitrogen atmosphere. The crude product was purified by Prep-HPLC with the following conditions (Column, XBridge Prep C18 OBD Column, 19*150 mm 5 um; mobile phase, Water (0.1% NH₃H₂O) and ACN (10% PhaseB up to 100% in 20 min); Detector, UV 254 nm. This resulted in tert-butyl (2S)-2-{[1-cyano-2-(5-{1′-methyl-2,3-dihydrospiro[indene-1,4′-piperidin]-6-yl}thieno[3,2-b]thiophen-2-yl)ethyl]carbamoyl}-1,4-oxazocane-4-carboxylate (180 mg, 71.9%) as a white solid. LCMS (ES) [M+1]⁺ m/z: 649.

### Synthesis of (2S)—N-[1-cyano-2-(5-{1′-methyl-2,3-dihydrospiro[indene-1,4′-piperidin]-6-yl}thieno[3,2-b]thiophen-2-yl)ethyl]-1,4-oxazocane-2-carboxamide

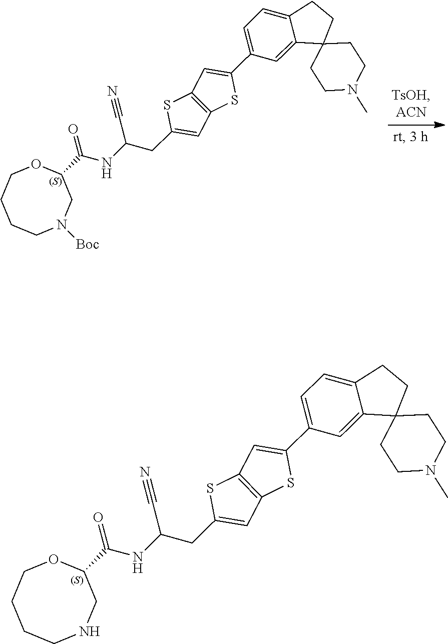

To a stirred solution of tert-butyl (2S)-2-{[1-cyano-2-(5-{1′-methyl-2,3-dihydrospiro[indene-1,4′-piperidin]-6-yl}thieno[3,2-b]thiophen-2-yl)ethyl]carbamoyl}-1,4-oxazocane-4-carboxylate (180 mg, 0.277 mmol, 1.0 equiv) in ACN (3 mL) was added TsOH (143 mg, 0.831 mmol, 3.0 equiv). The resulting mixture was stirred for 3 h at room temperature. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in (2S)—N-[1-cyano-2-(5-{1′-methyl-2,3-dihydrospiro[indene-1,4′-piperidin]-6-yl}thieno[3,2-b]thiophen-2-yl)ethyl]-1,4-oxazocane-2-carboxamide (90 mg, 59.1%) as a white solid. LCMS (ES) [M+1]⁺ m/z: 549.

### Synthesis of (2S)—N-[(1S)-1-cyano-2-(5-{1′-methyl-2,3-dihydrospiro[indene-1,4′-piperidin]-6-yl}thieno[3,2-b]thiophen-2-yl)ethyl]-1,4-oxazocane-2-carboxamide and (2S)—N-[(1R)-1-cyano-2-(5-{1′-methyl-2,3-dihydrospiro[indene-1,4′-piperidin]-6-yl}thieno[3,2-b]thiophen-2-yl)ethyl]-1,4-oxazocane-2-carboxamide

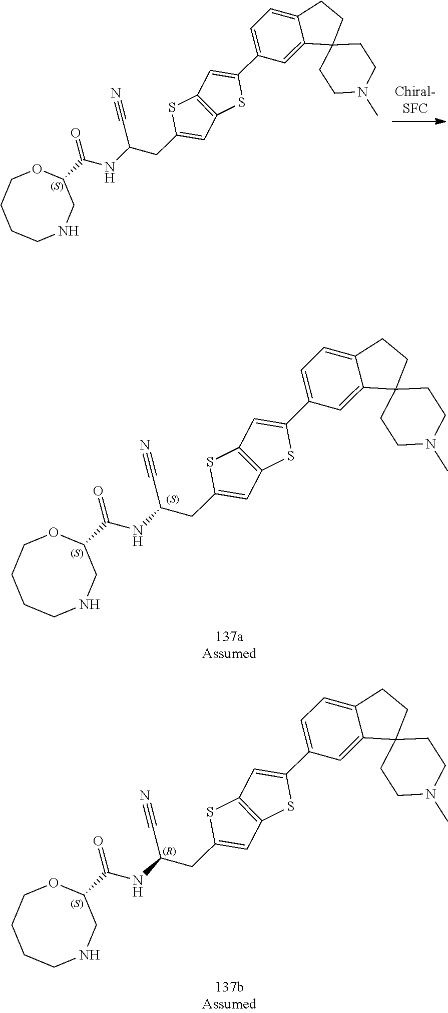

The product (2S)—N-[1-cyano-2-(5-{1′-methyl-2,3-dihydrospiro[indene-1,4′-piperidin]-6-yl}thieno[3,2-b]thiophen-2-yl)ethyl]-1,4-oxazocane-2-carboxamide (90 mg, 0.164 mmol, 1 equiv) was purified by Prep-CHIRAL-SFC with the following conditions: Column: XA-CHIRAL ART Cellulose-SZ, 3*25 cm 5 um; Mobile Phase A: CO₂, Mobile Phase B: MEOH:DCM=2:1(0.1% 2M NH₃-MeOH); Flow rate: 80 mL/min; Gradient: isocratic 50% B; Column Temperature (° C.): 35; Back Pressure (bar): 100; Wave Length: 220 nm; RT1(min): 7.26; RT2(min): 10.39; Sample Solvent: MEOH; Injection Volume: 4.5 mL. This resulted in (2S)—N-[(1S)-1-cyano-2-(5-{1′-methyl-2,3-dihydrospiro[indene-1,4′-piperidin]-6-yl}thieno[3,2-b]thiophen-2-yl)ethyl]-1,4-oxazocane-2-carboxamide (30 mg, 33.3%) and (2S)—N-[(1R)-1-cyano-2-(5-{1′-methyl-2,3-dihydrospiro[indene-1,4′-piperidin]-6-yl}thieno[3,2-b]thiophen-2-yl)ethyl]-1,4-oxazocane-2-carboxamide (30 mg, 33.3%) as a white solid.

137a:

LCMS (ES) [M+1]⁺ m/z: 549.

¹H NMR (300 MHz, DMSO-d6) δ 8.72 (d, J=8.5 Hz, 1H), 7.78 (s, 1H), 7.46 (s, 1H), 7.44-7.37 (m, 1H), 7.29 (s, 1H), 7.23 (d, J=7.9 Hz, 1H), 4.95 (q, J=8.0 Hz, 1H), 4.04-3.91 (m, 1H), 3.81 (dd, J=9.8, 2.8 Hz, 1H), 3.67-3.57 (m, 1H), 3.55-3.36 (m, 2H), 3.10 (dd, J=14.1, 2.8 Hz, 1H), 3.05-2.91 (m, 1H), 2.84 (t, J=7.2 Hz, 2H), 2.77-2.55 (m, 3H), 2.50-2.38 (m, 1H), 2.21 (s, 3H), 2.13-1.81 (m, 7H), 1.56-1.40 (m, 5H).

137b:

LCMS (ES) [M+1]⁺ m/z: 549.

1H NMR (300 MHz, DMSO-d6) δ 8.69 (d, J=8.6 Hz, 1H), 7.79 (s, 1H), 7.46 (s, 1H), 7.42 (d, J=7.8 Hz, 1H), 7.28 (s, 1H), 7.23 (d, J=7.8 Hz, 1H), 4.99 (q, J=8.0 Hz, 1H), 4.02-3.83 (m, 2H), 3.72-3.61 (m, 1H), 3.56-3.37 (m, 2H), 3.06-2.89 (m, 2H), 2.84 (t, J=7.2 Hz, 2H), 2.77-2.52 (m, 3H), 2.32 (dd, J=14.0, 9.7 Hz, 1H), 2.21 (s, 3H), 2.15-1.74 (m, 7H), 1.70-1.35 (m, 5H).

### Example 29. Synthesis of Compound 138a, 138b and 138c: (2S)—N-[(1S)-1-cyano-2-[5-(1-ethyl-2-oxo-3H-indol-6-yl)thieno[3,2-b]thiophen-2-yl]ethyl]-1,4-oxazocane-2-carboxamide and (2S)—N-[(1R)-1-cyano-2-[5-(1-ethyl-2-oxo-3H-indol-6-yl)thieno[3,2-b]thiophen-2-yl]ethyl]-1,4-oxazocane-2-carboxamide

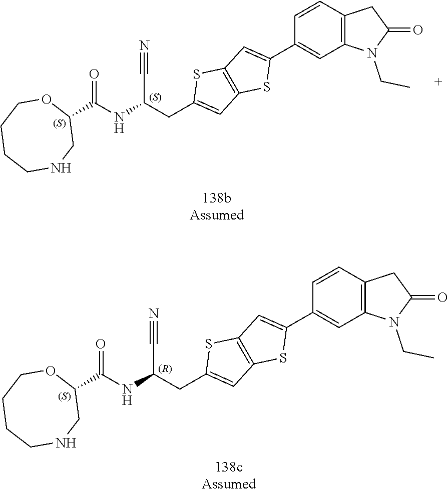

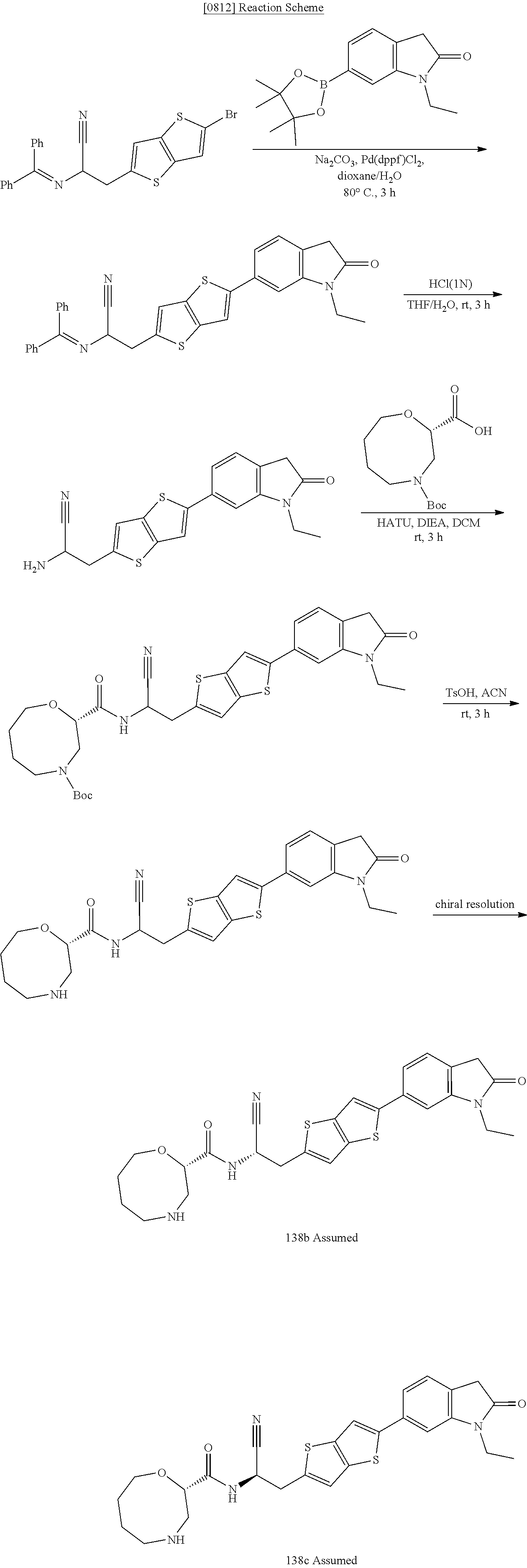

### Experimental Details

### Synthesis of 2-[(diphenylmethylidene)amino]-3-[5-(1-ethyl-2-oxo-3H-indol-6-yl)thieno[3,2-b]thiophen-2-yl]propanenitrile

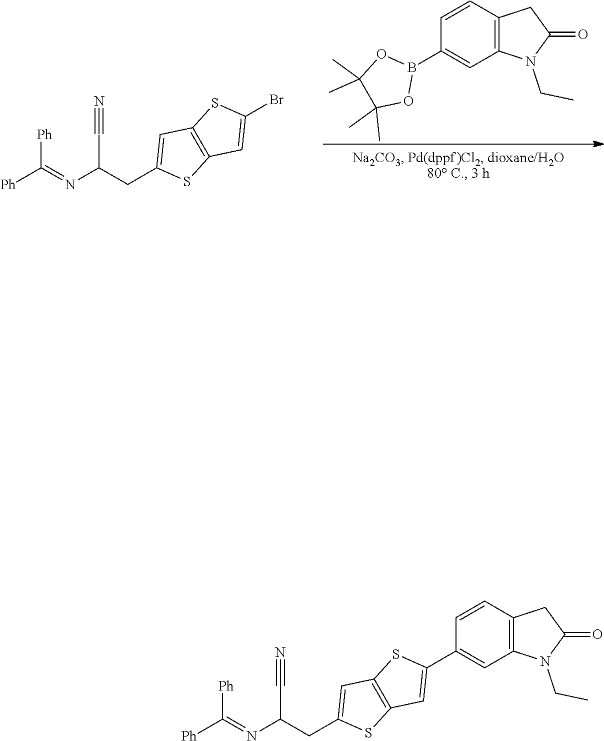 [SMILES: N#CC(Cc1cc2sc(Br)cc2s1)N=C(c1ccccc1)c1ccccc1 ; CCN1C(=O)Cc2ccc(B3OC(C)(C)C(C)(C)O3)cc21 ; "Na2CO3, Pd(dppf)Cl2, dioxane/H2O80° C., 3 h" ; CCN1C(=O)Cc2ccc(-c3cc4sc(CC(C#N)N=C(c5ccccc5)c5ccccc5)cc4s3)cc21]

To a solution of 1-ethyl-6-(4,4,5,5-tetramethyl-1,3,2-dioxaborolan-2-yl)-3H-indol-2-one (400 mg, 1.39 mmol, 1.0 equiv), 3-{5-bromothieno[3,2-b]thiophen-2-yl}-2-[(diphenylmethylidene)amino]propanenitrile (628 mg, 1.39 mmol, 1.0 equiv) in 1,4-dioxane (5 mL), H₂O (0.5 mL), Na₂CO₃ (295 mg, 2.78 mmol, 2.0 equiv) and Pd(dppf)Cl₂ (101 mg, 0.1 mmol, 0.1 equiv) was stirred for 3 h at 80° C. under nitrogen atmosphere. The reaction was cooled to room temperature, concentrated to remove the solvent. The residue was purified by silica gel column chromatography, eluted with PE/THF (2:1) to afford 2-[(diphenylmethylidene)amino]-3-[5-(1-ethyl-2-oxo-3H-indol-6-yl)thieno[3,2-b]thiophen-2-yl]propanenitrile (250 mg, 33.7%) as a yellow semi-solid. LCMS (ES, m/z): [M+H]⁺: 532.

### Synthesis of 2-amino-3-[5-(1-ethyl-2-oxo-3H-indol-6-yl)thieno[3,2-b]thiophen-2-yl]propanenitrile

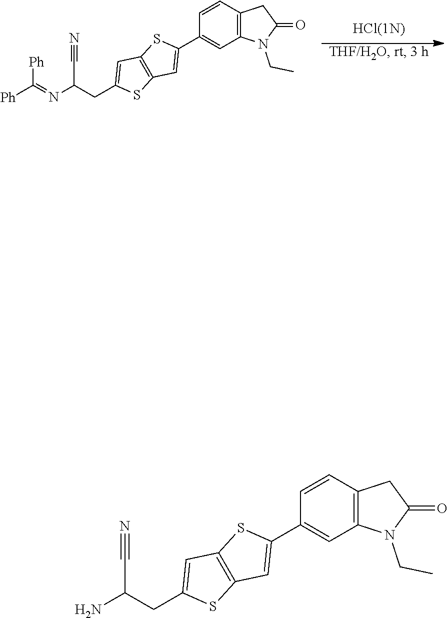 [SMILES: CCN1C(=O)Cc2ccc(-c3cc4sc(CC(C#N)N=C(c5ccccc5)c5ccccc5)cc4s3)cc21 ; "HCl(1N)" ; "THF/H2O, rt, 3 h" ; CCN1C(=O)Cc2ccc(-c3cc4sc(CC(N)C#N)cc4s3)cc21]

A solution of 2-[(diphenylmethylidene)amino]-3-[5-(1-ethyl-2-oxo-3H-indol-6-yl)thieno[3,2-b]thiophen-2-yl]propanenitrile (250 mg, 0.47 mmol, 1.0 equiv) and HCl (1 N, 1 mL) in THE (15 mL), H₂O (1 mL) was stirred for 3 h at room temperature. The mixture was basified to pH 10 with saturated K₂CO₃ (aq.). The resulting mixture was extracted with EtOAc (3×30 mL). The combined organic layers was washed with brine (3×30 mL), dried over anhydrous Na₂SO₄. After filtration, the filtrate was concentrated under reduced pressure. The residue was purified by silica gel column chromatography, eluted with PE/EA (1:2) to afford 2-amino-3-[5-(1-ethyl-2-oxo-3H-indol-6-yl)thieno[3,2-b]thiophen-2-yl]propanenitrile (150 mg, 86.8%) as off-white semi-solid. LCMS (ES, m/z): [M+H]⁺: 368.

### Synthesis of tert-butyl (2S)-2-({1-cyano-2-[5-(1-ethyl-2-oxo-3H-indol-6-yl)thieno[3,2-b]thiophen-2-yl]ethyl}carbamoyl)-1,4-oxazocane-4-carboxylate

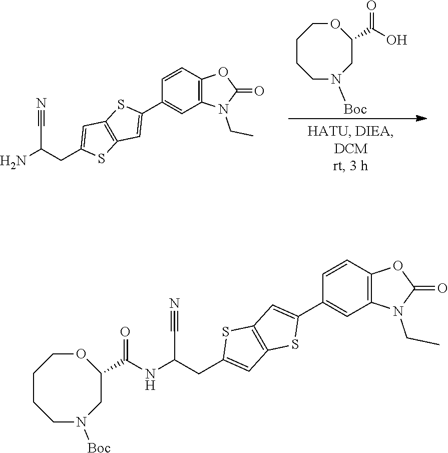 [SMILES: CCn1c(=O)oc2ccc(-c3cc4sc(CC(N)C#N)cc4s3)cc21 ; CC(C)(C)OC(=O)N1CCCCO[C@H](C(=O)O)C1 ; CCn1c(=O)oc2ccc(-c3cc4sc(CC(C#N)NC(=O)[C@@H]5CN(C(=O)OC(C)(C)C)CCCCO5)cc4s3)cc21]

To a stirred solution of (2S)-4-(tert-butoxycarbonyl)-1,4-oxazocane-2-carboxylic acid (100 mg, 0.38 mmol, 1.0 equiv) and 2-amino-3-[5-(1-ethyl-2-oxo-3H-indol-6-yl)thieno[3,2-b]thiophen-2-yl]propanenitrile (142 mg, 0.38 mmol, 1.0 equiv), DIEA (149 mg, 1.15 mmol, 3.0 equiv) in DCM (3 mL) was added HATU (176 mg, 0.46 mmol, 1.2 equiv) in portions at 0° C. The resulting mixture was stirred for 3 h at 0° C. Concentrated to remove the solvent, the residue was purified by silica gel column chromatography, eluted with PE/THF (1:1) to afford tert-butyl (2S)-2-({1-cyano-2-[5-(1-ethyl-2-oxo-3H-indol-6-yl)thieno[3,2-b]thiophen-2-yl]ethyl}carbamoyl)-1,4-oxazocane-4-carboxylate (150 mg, 63.8%) as off-white semi-solid. LCMS (ES, m/z): [M+H]⁺: 609.

### Synthesis of (2S)—N-{1-cyano-2-[5-(1-ethyl-2-oxo-3H-indol-6-yl)thieno[3,2-b]thiophen-2-yl]ethyl}-1,4-oxazocane-2-carboxamide

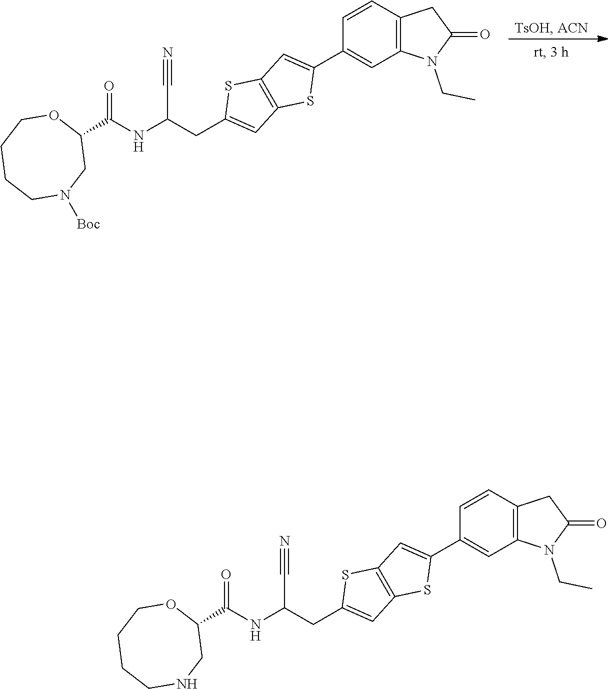

Into a 25 mL round-bottom flask were added tert-butyl (2S)-2-({1-cyano-2-[5-(1-ethyl-2-oxo-3H-indol-6-yl)thieno[3,2-b]thiophen-2-yl]ethyl}carbamoyl)-1,4-oxazocane-4-carboxylate (150 mg, 0.24 mmol, 1.0 equiv), acetonitrile (3 mL) and TsOH·H₂O (140 mg, 0.73 mmol, 3.0 equiv) at room temperature. The resulting mixture was stirred for 3 h at room temperature. The reaction solution was purified by reversed-phase flash chromatography with the following conditions: column, C₁₈ silica gel-120 g; mobile phase, MeCN in Water (0.1% NH₃·H₂O), 10% to 50% gradient in 15 min; detector, UV 254 nm. This resulted in (2S)—N-{1-cyano-2-[5-(1-ethyl-2-oxo-3H-indol-6-yl)thieno[3,2-b]thiophen-2-yl]ethyl}-1,4-oxazocane-2-carboxamide (60 mg, 47.8%) as white solid. LCMS (ES, m/z): [M+H]⁺: 509.

### Synthesis of (2S)—N-[(1S)-1-cyano-2-[5-(1-ethyl-2-oxo-3H-indol-6-yl)thieno[3,2-b]thiophen-2-yl]ethyl]-1,4-oxazocane-2-carboxamide and (2S)—N-[(1R)-1-cyano-2-[5-(1-ethyl-2-oxo-3H-indol-6-yl)thieno[3,2-b]thiophen-2-yl]ethyl]-1,4-oxazocane-2-carboxamide

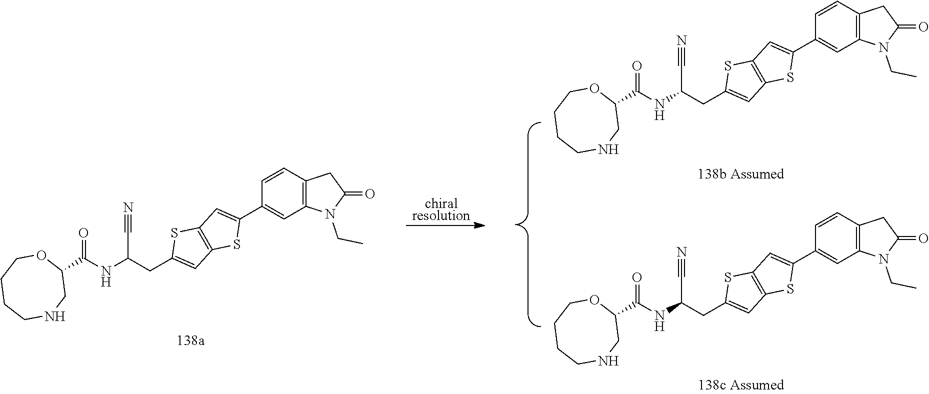 [SMILES: CCN1C(=O)Cc2ccc(-c3cc4sc(C[C@@H](C#N)NC(=O)[C@@H]5CNCCCCO5)cc4s3)cc21 ; CCN1C(=O)Cc2ccc(-c3cc4sc(CC(C#N)NC(=O)[C@@H]5CNCCCCO5)cc4s3)cc21 ; "chiralresolution" ; CCN1C(=O)Cc2ccc(-c3cc4sc(C[C@H](C#N)NC(=O)[C@@H]5CNCCCCO5)cc4s3)cc21]

(2S)—N-{1-cyano-2-[5-(1-ethyl-2-oxo-3H-indol-6-yl)thieno[3,2-b]thiophen-2-yl]ethyl}-1,4-oxazocane-2-carboxamide (60 mg, 0.12 mmol, 1.0 equiv) was purified by Prep-HPLC with the following conditions Column: CHIRALPAK IC, 2*25 cm, 5 m; Mobile Phase A: Hex:DCM=1:1—HPLC, Mobile Phase B: EtOH (0.1% 2M NH₃-MeOH); Flow rate: 25 mL/min; Gradient: isocratic 50; Wave Length: 254 nm; RT1(min): 7.6; RT2(min): 11.1; Sample Solvent: EtOH: DCM=1: 1—HPLC; Injection Volume: 1.5 mL; Number Of Runs: 5. This resulted in (2S)—N-[(1S)-1-cyano-2-[5-(1-ethyl-2-oxo-3H-indol-6-yl)thieno[3,2-b]thiophen-2-yl]ethyl]-1,4-oxazocane-2-carboxamide (15.2 mg, 25.3%) as a white solid and (2S)—N-[(1R)-1-cyano-2-[5-(1-ethyl-2-oxo-3H-indol-6-yl)thieno[3,2-b]thiophen-2-yl]ethyl]-1,4-oxazocane-2-carboxamide (17.6 mg, 29.3%) as a white solid.

### Analytical Data

LCMS (ES, m/z): [M+H]⁺: 509.0

¹H NMR (400 MHz, DMSO-d₆) δ 8.80 (d, J=8.7 Hz, 1H), 7.92 (s, 1H), 7.38-7.26 (m, 4H), 5.06-4.98 (m, 1H), 4.07-3.87 (m, 2H), 3.77 (q, J=7.2 Hz, 2H), 3.72-3.65 (m, 1H), 3.58 (s, 2H), 3.54-3.44 (m, 2H), 3.05-2.94 (m, 2H), 2.59-2.56 (m, 1H), 2.48-2.35 (m, 1H), 1.94-1.76 (m, 1H), 1.62-1.49 (m, 3H), 1.19 (t, J=7.1 Hz, 3H).

¹H NMR (400 MHz, DMSO-d₆) δ 8.76 (d, J=8.4 Hz, 1H), 7.92 (s, 1H), 7.40-7.25 (m, 4H), 5.03-4.95 (m, 1H), 4.03-3.92 (m, 1H), 3.85-3.74 (m, 3H), 3.68-3.62 (m, 1H), 3.58 (s, 2H), 3.52-3.46 (m, 2H), 3.15-3.10 (m, 1H), 3.03-2.96 (m, 1H), 2.66-2.56 (m, 1H), 2.48-2.42 (m, 1H), 1.96-1.86 (m, 1H), 1.59-1.47 (m, 3H), 1.19 (t, J=7.1 Hz, 3H).

### Example 30. Synthesis of Compound: 139a, 139b and 139c

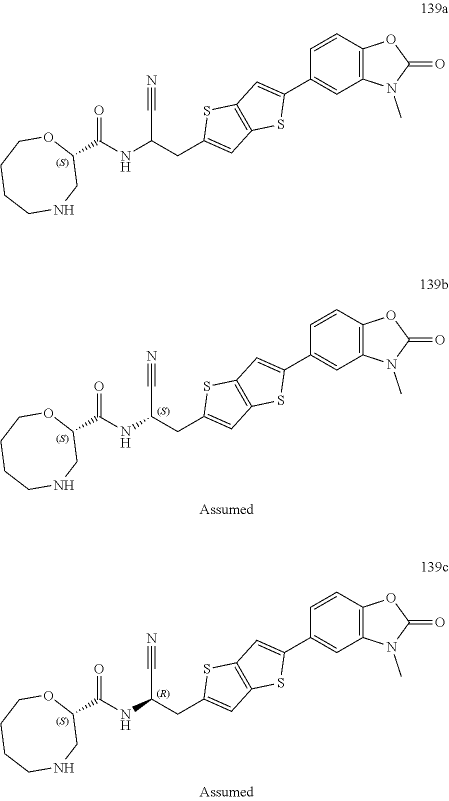

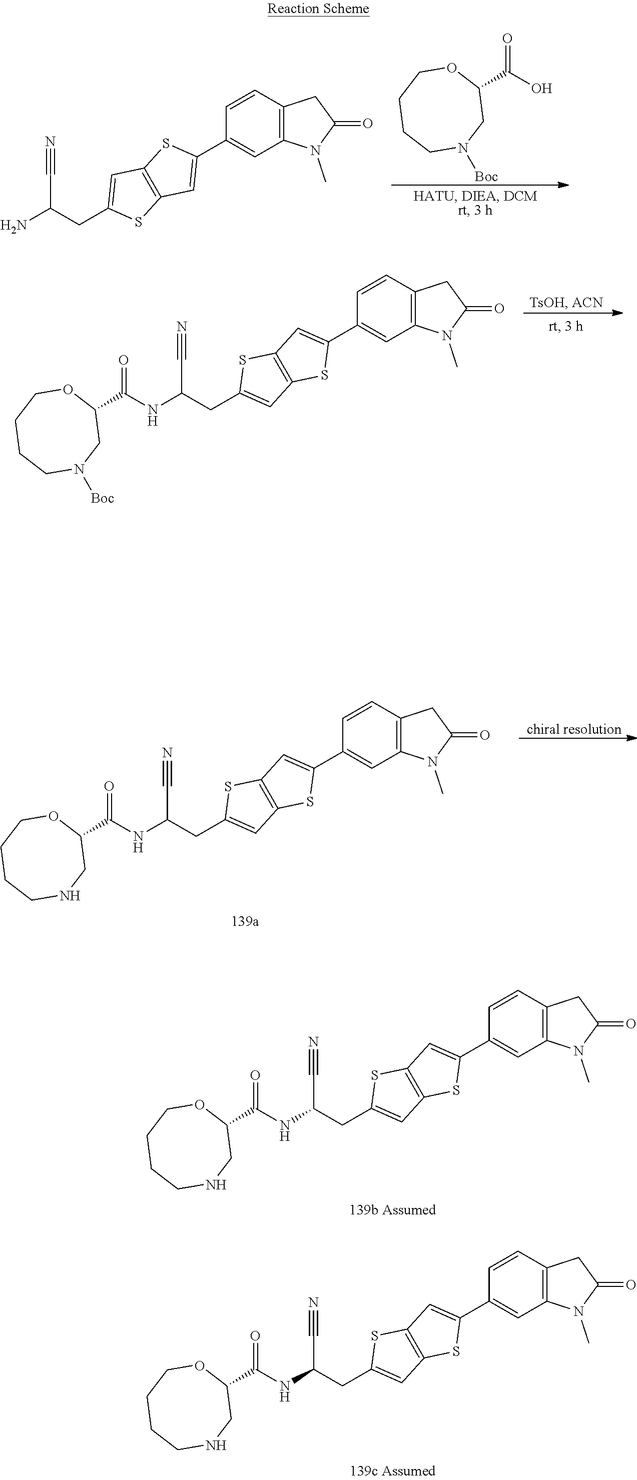

### Experimental Details

### Synthesis of tert-butyl (2S)-2-({1-cyano-2-[5-(3-methyl-2-oxo-1,3-benzoxazol-5-yl)thieno[3,2-b]thiophen-2-yl]ethyl}carbamoyl)-1,4-oxazocane-4-carboxylate

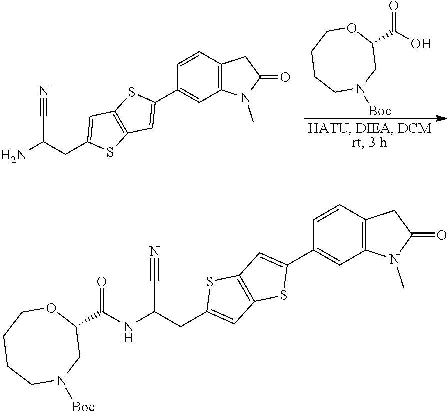

To a stirred solution of (2S)-4-(tert-butoxycarbonyl)-1,4-oxazocane-2-carboxylic acid (90 mg, 0.34 mmol, 1.0 equiv) and 2-amino-3-[5-(3-methyl-2-oxo-1,3-benzoxazol-5-yl)thieno[3,2-b]thiophen-2-yl]propanenitrile (136 mg, 0.38 mmol, 1.1 equiv), DIEA (135 mg, 1.04 mmol, 3.0 equiv) in DCM (3 mL) was added HATU (158 mg, 0.41 mmol, 1.2 equiv) in portions at 0° C. The resulting mixture was stirred for 3 h at 0° C. The reaction solution was purified by silica gel column chromatography, eluted with PE/THF (1:1) to afford tert-butyl (2S)-2-({1-cyano-2-[5-(3-methyl-2-oxo-1,3-benzoxazol-5-yl)thieno[3,2-b]thiophen-2-yl]ethyl}carbamoyl)-1,4-oxazocane-4-carboxylate (170 mg, 82.0%) as white semi-solid. LCMS (ES, m/z): [M+H]⁺: 597.

### Synthesis of (2S)—N-{1-cyano-2-[5-(3-methyl-2-oxo-1,3-benzoxazol-5-yl)thieno[3,2-b]thiophen-2-yl]ethyl}-1,4-oxazocane-2-carboxamide

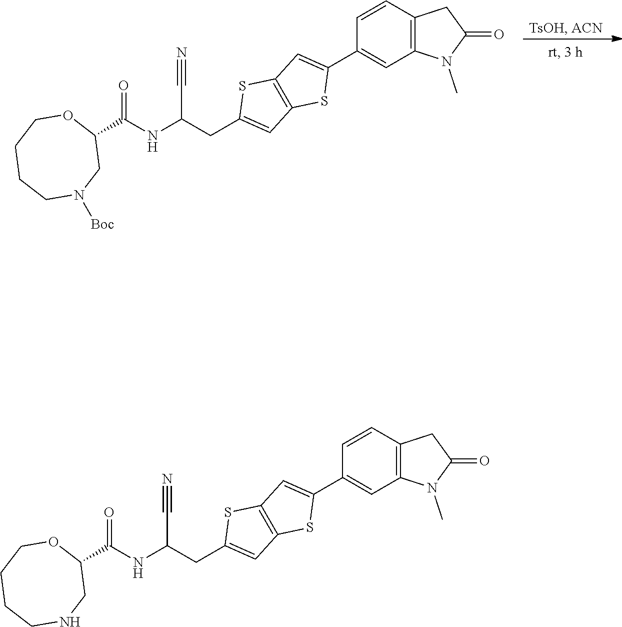

Into a 25 mL round-bottom flask were added tert-butyl (2S)-2-({1-cyano-2-[5-(3-methyl-2-oxo-1,3-benzoxazol-5-yl)thieno[3,2-b]thiophen-2-yl]ethyl}carbamoyl)-1,4-oxazocane-4-carboxylate (170 mg, 0.28 mmol, 1.0 equiv), Acetonitrile (3 mL) and TsOH·H₂O (147 mg, 0.85 mmol, 3.0 equiv) at room temperature. The resulting mixture was stirred for 3 h at room temperature. The reaction solution was purified by reversed-phase flash chromatography with the following conditions: column, C18 silica gel-120 g; mobile phase, MeCN in Water (0.1% NH₃·H₂O), 10% to 50% gradient in 15 min; detector, UV 254 nm. This resulted in (2S)—N-{1-cyano-2-[5-(3-methyl-2-oxo-1,3-benzoxazol-5-yl)thieno[3,2-b]thiophen-2-yl]ethyl}-1,4-oxazocane-2-carboxamide (80 mg, 56.5%) as white solid. LCMS (ES, m/z): [M+H]⁺: 497.

### Synthesis of (2S)—N-[(1S)-1-cyano-2-[5-(3-methyl-2-oxo-1,3-benzoxazol-5-yl)thieno[3,2-b]thiophen-2-yl]ethyl]-1,4-oxazocane-2-carboxamide; (2S)—N-[(1R)-1-cyano-2-[5-(3-methyl-2-oxo-1,3-benzoxazol-5-yl)thieno[3,2-b]thiophen-2-yl]ethyl]-1,4-oxazocane-2-carboxamide

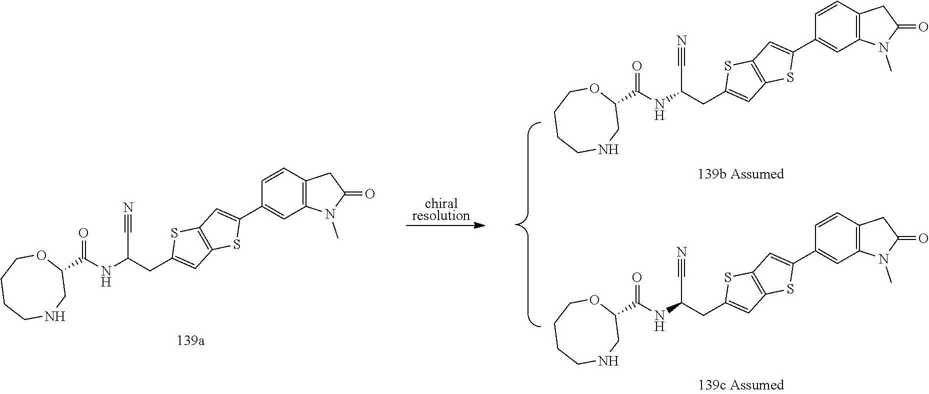 [SMILES: CN1C(=O)Cc2ccc(-c3cc4sc(C[C@@H](C#N)NC(=O)[C@@H]5CNCCCCO5)cc4s3)cc21 ; CN1C(=O)Cc2ccc(-c3cc4sc(CC(C#N)NC(=O)[C@@H]5CNCCCCO5)cc4s3)cc21 ; "chiralresolution" ; CN1C(=O)Cc2ccc(-c3cc4sc(C[C@H](C#N)NC(=O)[C@@H]5CNCCCCO5)cc4s3)cc21]

(2S)—N-{1-cyano-2-[5-(3-methyl-2-oxo-1,3-benzoxazol-5-yl)thieno[3,2-b]thiophen-2-yl]ethyl}-1,4-oxazocane-2-carboxamide (80 mg, 0.16 mmol, 1.0 equiv) was purified by Prep-HPLC with the following conditions Column: XA-CHIRALPAK IC, 2*25 cm, 5 m; Mobile Phase A: Hex:DCM=1:1—HPLC, Mobile Phase B: EtOH (0.1% 2M NH₃-MeOH); Flow rate: 25 mL/min; Gradient: isocratic 50; Wave Length: 254 nm; RT1(min): 9.0; RT2(min): 14.4; Sample Solvent: EtOH:DCM=1:1 (0.1% 2M NH₃-MeOH); Injection Volume: 3.5 mL; Number Of Runs: 5. This resulted in (2S)—N-[(1S)-1-cyano-2-[5-(3-methyl-2-oxo-1,3-benzoxazol-5-yl)thieno[3,2-b]thiophen-2-yl]ethyl]-1,4-oxazocane-2-carboxamide (17.6 mg, 22%) as white solid and (2S)—N-[(1R)-1-cyano-2-[5-(3-methyl-2-oxo-1,3-benzoxazol-5-yl)thieno[3,2-b]thiophen-2-yl]ethyl]-1,4-oxazocane-2-carboxamide (20.5 mg, 25.6%) as white solid.

### Analytical Data

LCMS (ES, m/z): [M+H]⁺: 497.0

¹H NMR (400 MHz, DMSO-d₆) δ 8.72 (d, J=8.5 Hz, 1H), 7.85 (s, 1H), 7.61 (d, J=1.9 Hz, 1H), 7.45-7.37 (m, 2H), 7.33 (s, 1H), 5.02 (q, J=8.0 Hz, 1H), 4.09-3.87 (m, 2H), 3.72-3.65 (m, 1H), 3.55-3.46 (m, 2H), 3.40 (s, 3H), 3.06-2.92 (m, 2H), 2.65-2.57 (m, 1H), 2.37-2.30 (m, 1H), 1.95-1.84 (m, 1H), 1.62-1.48 (m, 3H).

¹H NMR (400 MHz, DMSO-d₆) δ 8.76 (d, J=8.5 Hz, 1H), 7.85 (d, J=1.8 Hz, 1H), 7.61 (d, J=1.8 Hz, 1H), 7.46-7.36 (m, 2H), 7.35 (s, 1H), 5.04-4.93 (m, 1H), 4.03-3.93 (m, 1H), 3.87-3.83 (m, 1H), 3.68-3.62 (m, 1H), 3.55-3.44 (m, 2H), 3.40 (s, 3H), 3.14 (dd, J=14.1, 3.0 Hz, 1H), 3.04-2.96 (m, 1H), 2.66-2.56 (m, 2H), 1.95-1.86 (m, 1H), 1.60-1.47 (m, 3H).

### Example 31. Synthesis of Compounds 141a, 141b and 141c: (2S,7R)—N-[(1S)-1-cyano-2-(7-fluoro-2-{1′-methyl-2,3-dihydrospiro[indene-1,4′-piperidin]-6-yl}-1-benzothiophen-6-yl)ethyl]-7-methoxy-1,4-oxazocane-2-carboxamide and (2S,7R)—N-[(1R)-1-cyano-2-(7-fluoro-2-{1′-methyl-2,3-dihydrospiro[indene-1,4′-piperidin]-6-yl}-1-benzothiophen-6-yl)ethyl]-7-methoxy-1,4-oxazocane-2-carboxamide

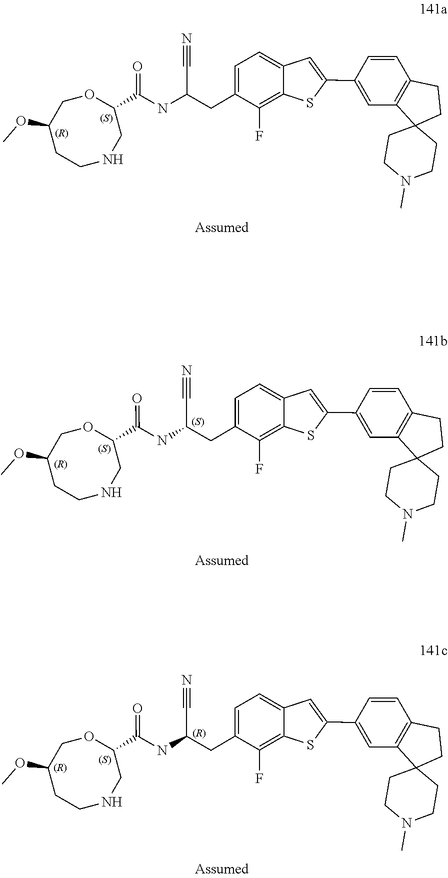

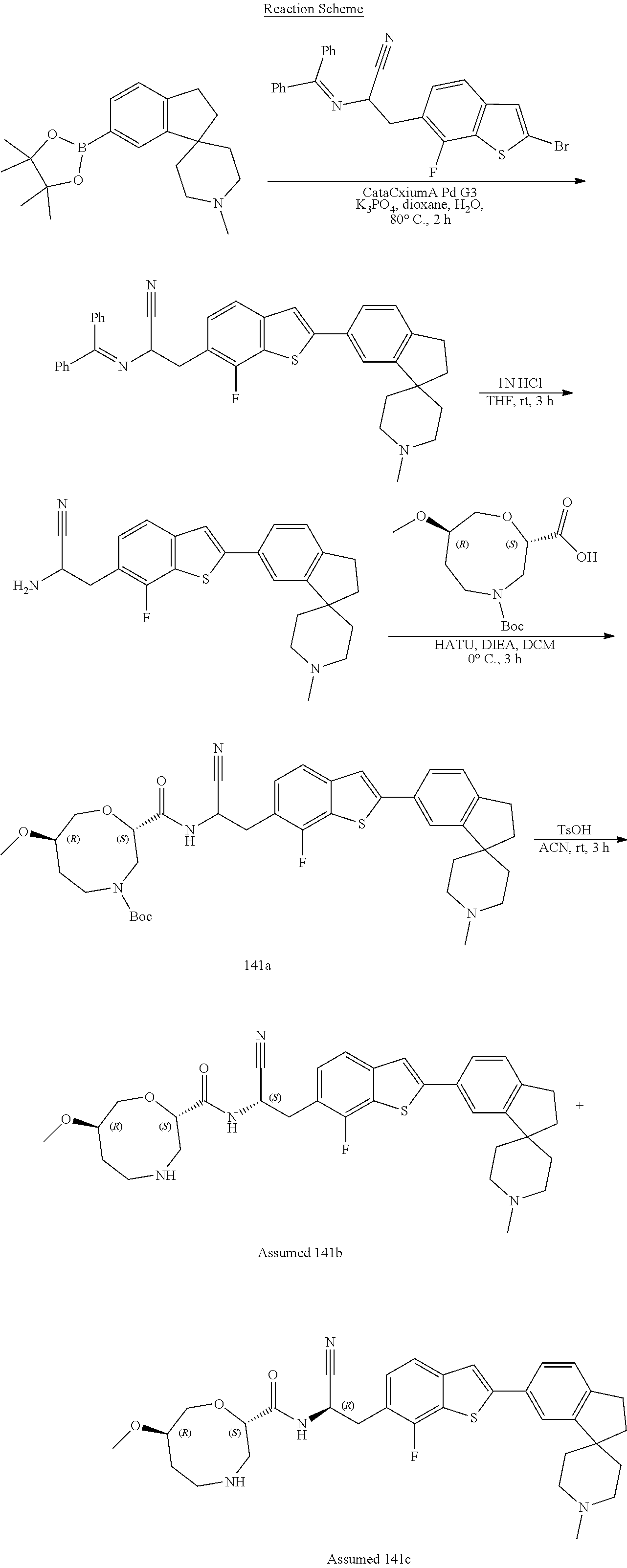

### Experimental Details

### Synthesis of 2-[(diphenylmethylidene)amino]-3-(7-fluoro-2-{1′-methyl-2,3-dihydrospiro[indene-1,4′-piperidin]-6-yl}-1-benzothiophen-6-yl)propanenitrile

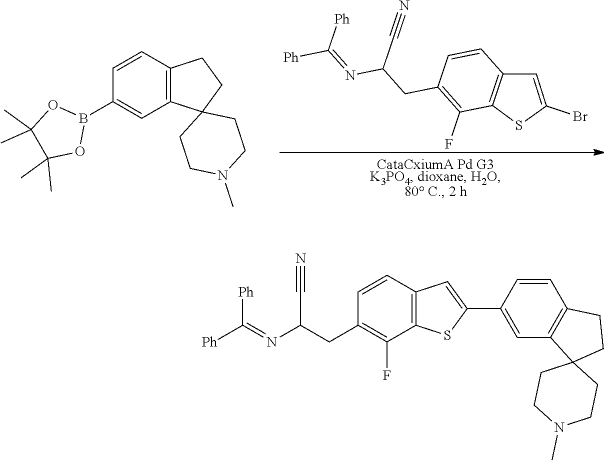

To a solution of 3-(2-bromo-7-fluoro-1-benzothiophen-6-yl)-2-[(diphenylmethylidene)amino]propanenitrile (900 mg, 1.94 mmol, 1.0 equiv) and 1′-methyl-6-(4,4,5,5-tetramethyl-1,3,2-dioxaborolan-2-yl)-2,3-dihydrospiro[indene-1,4′-piperidine](1.01 g, 3.10 mmol, 1.6 equiv) in dioxane (10 mL) and H₂O (1 mL), were added K₃PO₄ (1.23 g, 5.82 mmol, 3.0 equiv), CataCXium A Pd G3 (141 mg, 0.19 mmol, 0.1 equiv) in sequence. The mixture was stirred for 3 h at 80° C. under nitrogen atmosphere. The reaction was cooled to room temperature, concentrated to remove the solvent, the residue was purified by silica gel column chromatography, eluted with PE/THF (1:1) to afford 2-[(diphenylmethylidene)amino]-3-(7-fluoro-2-{1′-methyl-2,3-dihydrospiro[indene-1,4′-piperidin]-6-yl}-1-benzothiophen-6-yl)propanenitrile (540 mg) as a light yellow semi-solid. LCMS (ES, m/z): [M+H]⁺: 584.

### Synthesis of 2-amino-3-(7-fluoro-2-{1′-methyl-2,3-dihydrospiro[indene-1,4′-piperidin]-6-yl}-1-benzothiophen-6-yl)propanenitrile

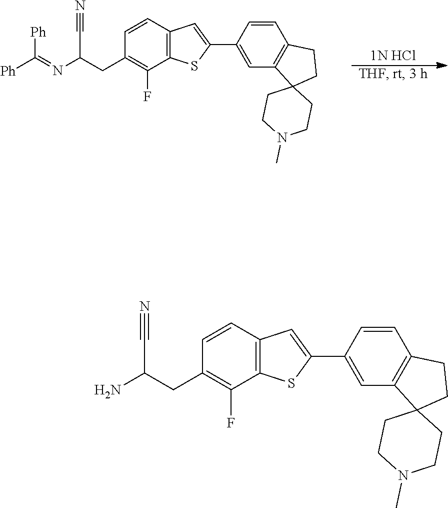 [SMILES: CN1CCC2(CCc3ccc(-c4cc5ccc(CC(C#N)N=C(c6ccccc6)c6ccccc6)c(F)c5s4)cc32)CC1 ; "1N HCl" ; "THF, rt, 3 h" ; CN1CCC2(CCc3ccc(-c4cc5ccc(CC(N)C#N)c(F)c5s4)cc32)CC1]

To a stirred solution of 2-[(diphenylmethylidene)amino]-3-(7-fluoro-2-{1′-methyl-2,3-dihydrospiro[indene-1,4′-piperidin]-6-yl}-1-benzothiophen-6-yl)propanenitrile (540 mg, 0.92 mmol, 1.0 equiv) in THF (25 mL) and H₂O (2.5 mL) was added HCl (1.4 mL) dropwise at room temperature. The resulting mixture was stirred at room temperature for 3 h. The mixture acidified to pH 8 with saturated NaHCO₃ (aq.). The resulting mixture was extracted with EtOAc (3×50 mL). The combined organic layer was dried over anhydrous Na₂SO₄. After filtration, the filtrate was concentrated under reduced pressure. The residue was purified by silica gel column chromatography, eluted with PE/THF (1:2) to afford 2-amino-3-(7-fluoro-2-{1′-methyl-2,3-dihydrospiro[indene-1,4′-piperidin]-6-yl}-1-benzothiophen-6-yl)propanenitrile (300 mg, 77.3%) as a light yellow oil. LCMS (ES, m/z): [M+H]⁺: 420.

### Synthesis of tert-butyl (2S,7R)-2-{[1-cyano-2-(7-fluoro-2-{1′-methyl-2,3-dihydrospiro[indene-1,4′-piperidin]-6-yl}-1-benzothiophen-6-yl)ethyl]carbamoyl}-7-methoxy-1,4-oxazocane-4-carboxylate

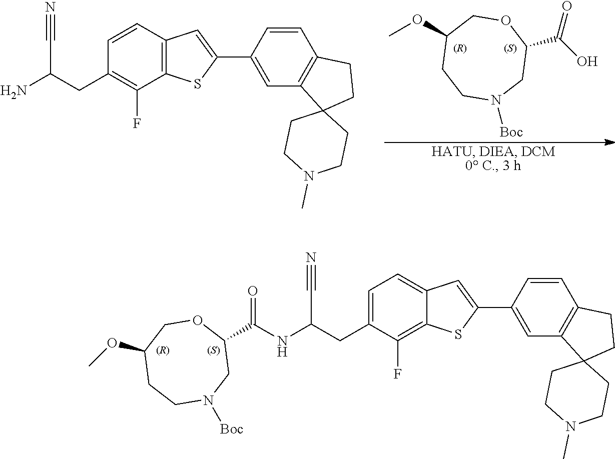

To a stirred mixture of (2S,7R)-4-(tert-butoxycarbonyl)-7-methoxy-1,4-oxazocane-2-carboxylic acid (137 mg, 0.47 mmol, 1.0 equiv), 2-amino-3-(7-fluoro-2-{1′-methyl-2,3-dihydrospiro[indene-1,4′-piperidin]-6-yl}-1-benzothiophen-6-yl)propanenitrile (200 mg, 0.47 mmol, 1.0 equiv) and DIEA (184 mg, 1.43 mmol, 3.0 equiv) in DCM (5 mL) were added HATU (217 mg, 0.57 mmol, 1.2 equiv) in portions at 0° C. The resulting mixture was stirred for 3 h at 0° C. Concentrated to remove the solvent, the residue was purified by silica gel column chromatography, eluted with PE/THF (1:1) to afford tert-butyl (2S,7R)-2-{[1-cyano-2-(7-fluoro-2-{1′-methyl-2,3-dihydrospiro[indene-1,4′-piperidin]-6-yl}-1-benzothiophen-6-yl)ethyl]carbamoyl}-7-methoxy-1,4-oxazocane-4-carboxylate (250 mg) as a light yellow oil. LCMS (ES, m/z): [M+H]⁺: 691.

### Synthesis of (2S,7R)—N-[(1S)-1-cyano-2-(7-fluoro-2-{1′-methyl-2,3-dihydrospiro[indene-1,4′-piperidin]-6-yl}-1-benzothiophen-6-yl)ethyl]-7-methoxy-1,4-oxazocane-2-carboxamide and (2S,7R)—N-[(1R)-1-cyano-2-(7-fluoro-2-{1′-methyl-2,3-dihydrospiro[indene-1,4′-piperidin]-6-yl}-1-benzothiophen-6-yl)ethyl]-7-methoxy-1,4-oxazocane-2-carboxamide

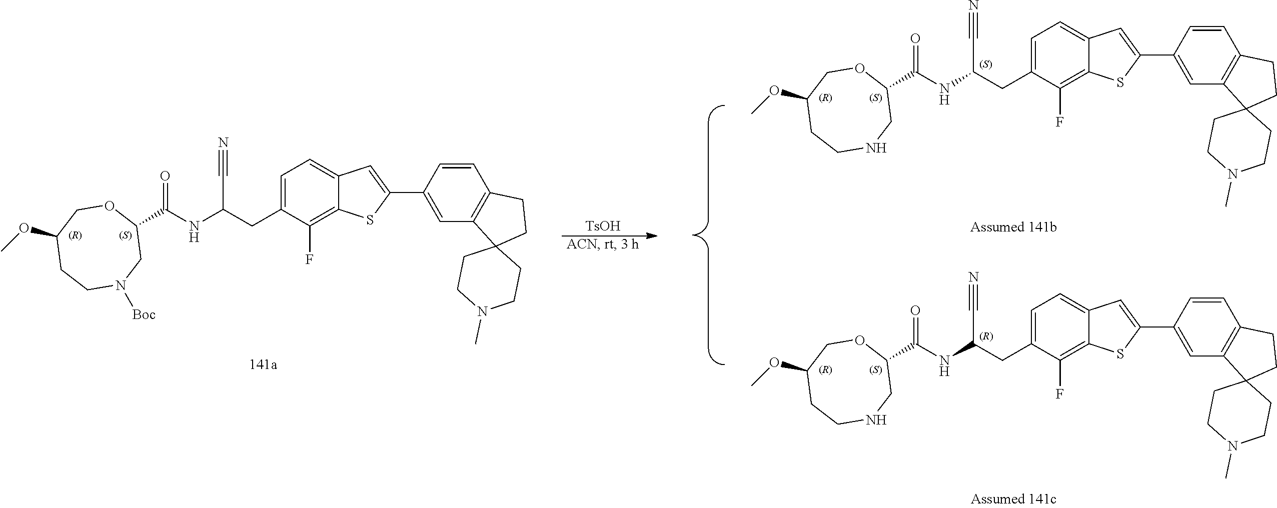

Into a 25 mL round-bottom flask were added tert-butyl (2S,7R)-2-{[1-cyano-2-(7-fluoro-2-{1′-methyl-2,3-dihydrospiro[indene-1,4′-piperidin]-6-yl}-1-benzothiophen-6-yl)ethyl]carbamoyl}-7-methoxy-1,4-oxazocane-4-carboxylate (250 mg, 0.36 mmol, 1.0 equiv), TsOH·H₂O (186 mg, 1.08 mmol, 3.0 equiv) and ACN (8 mL) at room temperature. The resulting mixture was stirred for 3 h at room temperature. The reaction solution was purified by reversed-phase flash chromatography with the following conditions: column, C18 silica gel-120 g; mobile phase, MeCN in Water (0.1% NH₃·H₂O), 10% to 70% gradient in 15 min; detector, UV 254 nm. The product was separated by Prep-CHIRAL-HPLC with the following conditions: Column: Chiralpak IC, 2*25 cm, 5 um; Mobile Phase A: Hex:DCM=1:1—HPLC, Mobile Phase B: EtOH (0.1% 2M NH₃-MEOH); Flow rate: 25 mL/min; Gradient: isocratic 10; Wave Length: 254 nm; RT1(min): 6.5; RT2(min): 8.5; Sample Solvent: EtOH; Injection Volume: 1 mL; Number Of Runs: 6. This resulted in (2S,7R)—N-[(1S)-1-cyano-2-(7-fluoro-2-{1′-methyl-2,3-dihydrospiro[indene-1,4′-piperidin]-6-yl}-1-benzothiophen-6-yl)ethyl]-7-methoxy-1,4-oxazocane-2-carboxamide (25 mg, 23%) and (2S,7R)—N-[(1R)-1-cyano-2-(7-fluoro-2-{1′-methyl-2,3-dihydrospiro[indene-1,4′-piperidin]-6-yl}-1-benzothiophen-6-yl)ethyl]-7-methoxy-1,4-oxazocane-2-carboxamide (25 mg, 23%) as a white solid.

### Analytical Data

LCMS (ES, m/z): [M+H]⁺: 591.2.

OA: ¹H NMR (400 MHz, DMSO-d6) δ 8.74 (d, J=8.7 Hz, 1H), 7.93 (d, J=3.8 Hz, 1H), 7.68-7.59 (m, 2H), 7.56 (dd, J=7.7, 1.8 Hz, 1H), 7.42-7.34 (m, 1H), 7.32 (d, J=7.9 Hz, 1H), 5.07 (q, J=8.3 Hz, 1H), 4.09 (dd, J=11.4, 4.3 Hz, 1H), 3.90 (dd, J=9.3, 3.0 Hz, 1H), 3.59 (dd, J=11.5, 8.5 Hz, 1H), 3.38 (dd, J=13.7, 7.4 Hz, 2H), 3.26 (s, 3H), 3.30-3.15 (m, 1H), 3.05-2.85 (m, 4H), 2.76 (d, J=11.3 Hz, 2H), 2.50-2.45 (m, 1H), 2.38-2.21 (m, 1H), 2.20 (s, 3H), 2.09 (t, J=11.7 Hz, 2H), 2.04-1.89 (m, 5H), 1.62-1.50 (m, 1H), 1.49 (d, J=12.5 Hz, 2H).

LCMS (ES, m/z): [M+H]⁺: 591.2.

¹H NMR (400 MHz, DMSO-d6) δ 8.75 (d, J=8.4 Hz, 1H), 7.94 (d, J=3.8 Hz, 1H), 7.64 (dd, J=4.9, 3.2 Hz, 2H), 7.57 (dd, J=7.8, 1.7 Hz, 1H), 7.38 (t, J=7.5 Hz, 1H), 7.32 (d, J=7.8 Hz, 1H), 5.01 (q, J=8.1 Hz, 1H), 4.14 (dd, J=11.4, 4.1 Hz, 1H), 3.82 (dd, J=9.4, 3.0 Hz, 1H), 3.52 (dd, J=11.5, 8.5 Hz, 1H), 3.45-3.32 (m, 2H), 3.24 (m, 4H), 3.15-3.00 (m, 2H), 2.90 (t, J=7.2 Hz, 2H), 2.76 (d, J=11.1 Hz, 2H), 2.63-2.49 (m, 2H), 2.24 (s, 3H), 2.09 (t, J=11.8 Hz, 2H), 2.01-1.88 (m, 5H), 1.63-1.49 (m, 3H).

### Example 32. Synthesis of compound 142a: (2S,7R)—N-{1-cyano-2-[5-(3-methyl-2-oxo-1,3-benzoxazol-5-yl)thieno[3,2-b]thiophen-2-yl]ethyl}-7-methoxy-1,4-oxazocane-2-carboxamide

### Reaction Scheme

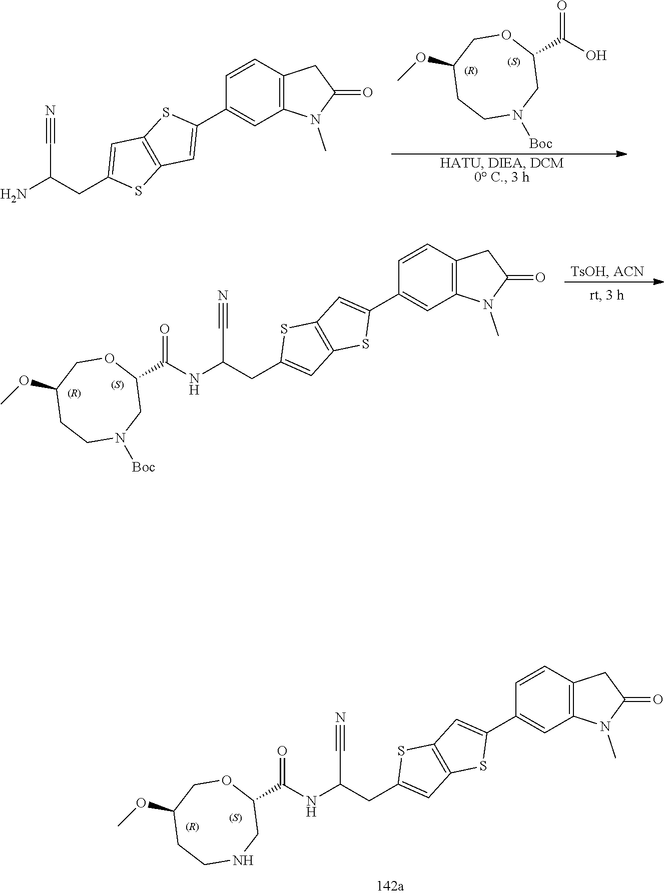

### Experimental Details

### Synthesis of tert-butyl (2S,7R)-2-({1-cyano-2-[5-(3-methyl-2-oxo-1,3-benzoxazol-5-yl)thieno[3,2-b]thiophen-2-yl]ethyl}carbamoyl)-7-methoxy-1,4-oxazocane-4-carboxylate

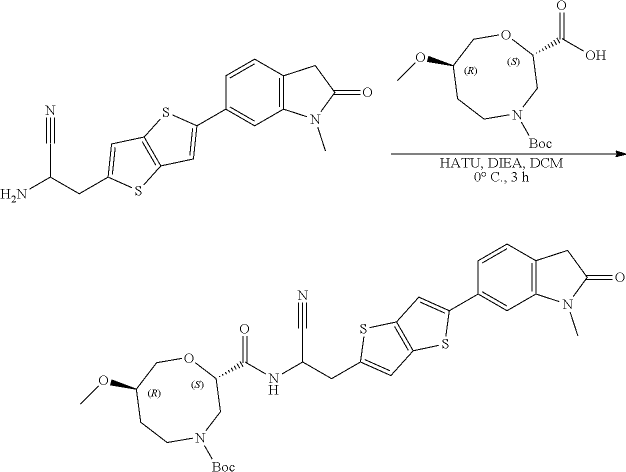

To a stirred solution of 2-amino-3-[5-(3-methyl-2-oxo-1,3-benzoxazol-5-yl)thieno[3,2-b]thiophen-2-yl]propanenitrile (70 mg, 0.19 mmol, 1.0 equiv) and (2S,7R)-4-(tert-butoxycarbonyl)-7-methoxy-1,4-oxazocane-2-carboxylic acid (68 mg, 0.23 mmol, 1.2 equiv), DIEA (76.36 mg, 0.591 mmol, 3.0 equiv) in DCM (3 mL) were added HATU (89 mg, 0.23 mmol, 1.2 equiv) in portions at 0° C. The resulting mixture was stirred for 3 h at 0° C. Concentrated under reduced pressure to remove the solvent, the residue was purified by silica gel column chromatography, eluted with PE/THF (1:1) to afford tert-butyl (2S,7R)-2-({1-cyano-2-[5-(3-methyl-2-oxo-1,3-benzoxazol-5-yl)thieno[3,2-b]thiophen-2-yl]ethyl}carbamoyl)-7-methoxy-1,4-oxazocane-4-carboxylate (100 mg, 81.0%) as off-white semi-solid. LCMS (ES, m/z): [M+H]⁺: 627.

### Synthesis of (2S,7R)—N-{1-cyano-2-[5-(3-methyl-2-oxo-1,3-benzoxazol-5-yl)thieno[3,2-b]thiophen-2-yl]ethyl}-7-methoxy-1,4-oxazocane-2-carboxamide

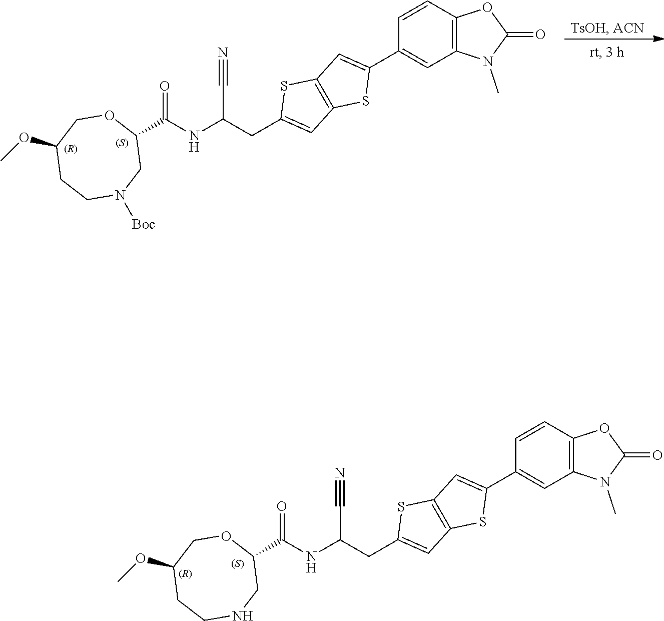

Into a 25 mL round-bottom flask were added tert-butyl (2S,7R)-2-({1-cyano-2-[5-(3-methyl-2-oxo-1,3-benzoxazol-5-yl)thieno[3,2-b]thiophen-2-yl]ethyl}carbamoyl)-7-methoxy-1,4-oxazocane-4-carboxylate (100 mg, 0.16 mmol, 1.0 equiv) in ACN (3 mL) and TsOH·H₂O (91 mg, 0.48 mmol, 3.0 equiv) at room temperature. The resulting mixture was stirred for 3 h at room temperature. The reaction solution was purified by reversed-phase flash chromatography with the following conditions: column, C₁₈ silica gel-120 g; mobile phase, MeCN in Water (0.1% NH₃·H₂O), 10% to 50% gradient in 10 min; detector, UV 254 nm. This resulted in (2S,7R)—N-{1-cyano-2-[5-(3-methyl-2-oxo-1,3-benzoxazol-5-yl)thieno[3,2-b]thiophen-2-yl]ethyl}-7-methoxy-1,4-oxazocane-2-carboxamide (16.8 mg, 20%) as white solid.

### Analytical Data

LCMS (ES, m/z): [M+H]⁺: 527.0

¹H NMR (400 MHz, DMSO-d₆) δ 8.78 (dd, J=13.5, 8.5 Hz, 1H), 7.85 (s, 1H), 7.61 (d, J=1.7 Hz, 1H), 7.43-7.36 (m, 2H), 7.35 (d, J=6.9 Hz, 1H), 5.07-4.96 (m, 1H), 4.19-4.09 (m, 1H), 3.97-3.88 (m, 1H), 3.66-3.42 (m, 3H), 3.41-3.37 (m, 4H), 3.26 (s, 3H), 3.18-2.96 (m, 2H), 2.58-2.54 (m, 1H), 2.43-2.37 (m, 1H), 1.98-1.88 (m, 1H), 1.64-1.54 (m, 1H).

### Example 33. Synthesis of Compound 143a, 143b and 143c: (2S,7R)—N—((S)-1-cyano-2-(6-(3-methyl-2-oxo-2,3-dihydrobenzo[d]oxazol-5-yl)-1H-indol-2-yl)ethyl)-7-methoxy-1,4-oxazocane-2-carboxamide and (2S,7R)—N—((R)-1-cyano-2-(6-(3-methyl-2-oxo-2,3-dihydrobenzo[d]oxazol-5-yl)-1H-indol-2-yl)ethyl)-7-methoxy-1,4-oxazocane-2-carboxamide

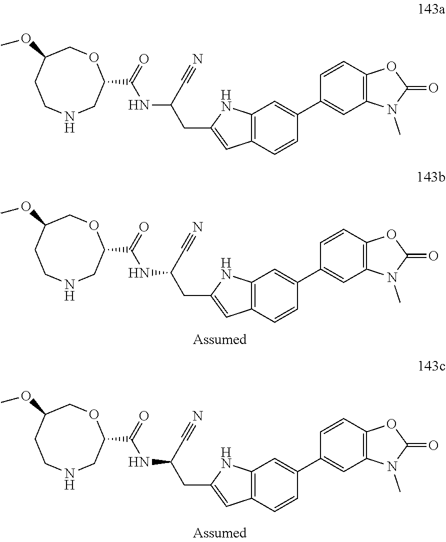 [SMILES: CO[C@@H]1CCNC[C@@H](C(=O)NC(C#N)Cc2cc3ccc(-c4ccc5oc(=O)n(C)c5c4)cc3[nH]2)OC1 ; CO[C@@H]1CCNC[C@@H](C(=O)N[C@H](C#N)Cc2cc3ccc(-c4ccc5oc(=O)n(C)c5c4)cc3[nH]2)OC1 ; CO[C@@H]1CCNC[C@@H](C(=O)N[C@@H](C#N)Cc2cc3ccc(-c4ccc5oc(=O)n(C)c5c4)cc3[nH]2)OC1]

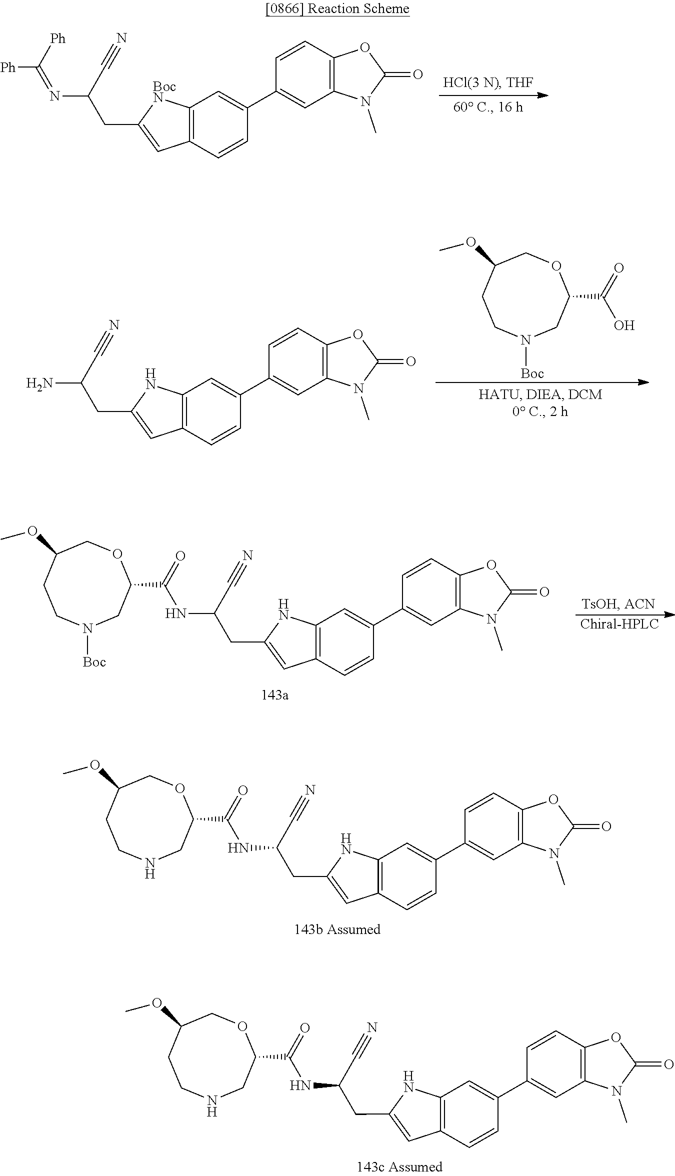

### Experimental Details.

### Synthesis of 2-amino-3-(6-(3-methyl-2-oxo-2,3-dihydrobenzo[d]oxazol-5-yl)-1H-indol-2-yl)propanenitrile

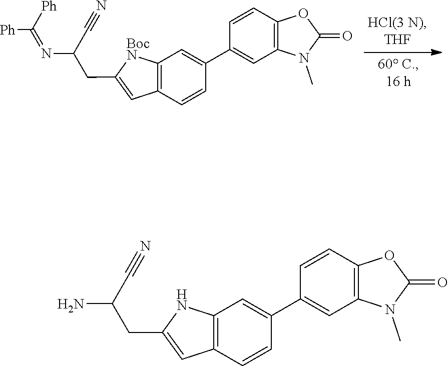 [SMILES: Cn1c(=O)oc2ccc(-c3ccc4cc(CC(C#N)N=C(c5ccccc5)c5ccccc5)n(C(=O)OC(C)(C)C)c4c3)cc21 ; Cn1c(=O)oc2ccc(-c3ccc4cc(CC(N)C#N)[nH]c4c3)cc21]

Into a 40 mL vial were added tert-butyl 2-{2-cyano-2-[(diphenylmethylidene)amino]ethyl}-6-(3-methyl-2-oxo-1,3-benzoxazol-5-yl)indole-1-carboxylate (500 mg, 0.84 mmol, 1.0 equiv), THF (10 mL) and HCl (3 M) (2 mL) at room temperature. The resulting mixture was stirred for 16 h at 60° C. The mixture was basified to pH 8 with saturated NaHCO₃ (aq.). The aqueous layer was extracted with EtOAc (20 mL×2). The combined organic phase was dried over anhydrous sodium sulfate. Filtered and the filtrate was concentrated under reduced pressure. The residue was purified by silica gel column chromatography, eluted with TIF/PE (from 10% to 100%) to afford 2-amino-3-[6-(3-methyl-2-oxo-1,3-benzoxazol-5-yl)-1H-indol-2-yl]propanenitrile (190 mg, 68.2%) as a yellow oil. LCMS (ES) [M+1]⁺ m/z: 333.

### Synthesis of tert-butyl (2S,7R)-2-((1-cyano-2-(6-(3-methyl-2-oxo-2,3-dihydrobenzo[d]oxazol-5-yl)-1H-indol-2-yl)ethyl)carbamoyl)-7-methoxy-1,4-oxazocane-4-carboxylate

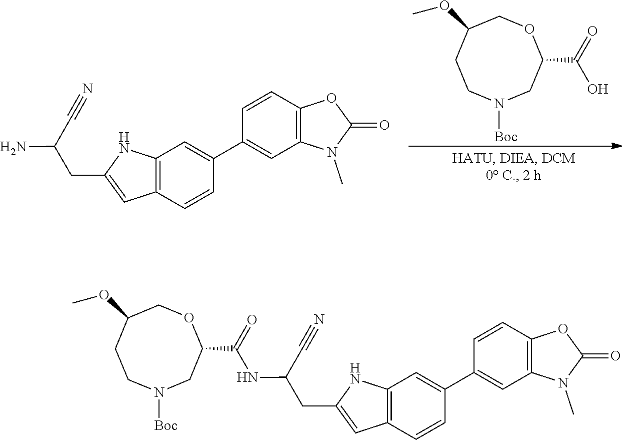 [SMILES: Cn1c(=O)oc2ccc(-c3ccc4cc(CC(N)C#N)[nH]c4c3)cc21 ; CO[C@@H]1CCN(C(=O)OC(C)(C)C)C[C@@H](C(=O)O)OC1 ; CO[C@@H]1CCN(C(=O)OC(C)(C)C)C[C@@H](C(=O)NC(C#N)Cc2cc3ccc(-c4ccc5oc(=O)n(C)c5c4)cc3[nH]2)OC1]

A solution of 2-amino-3-[6-(3-methyl-2-oxo-1,3-benzoxazol-5-yl)-1H-indol-2-yl]propanenitrile (170 mg, 0.51 mmol, 1.0 equiv), DIEA (198 mg, 1.53 mmol, 3.0 equiv) in DCM (5 mL), HATU (233 mg, 0.61 mmol, 1.2 equiv) was added at 0° C. The resulting mixture was stirred for 2 h at 0° C. Concentrated to remove the solvent, the residue was purified by silica gel column chromatography, eluted with PE/EA (1:2) to afford tert-butyl (2S,7R)-2-({1-cyano-2-[6-(3-methyl-2-oxo-1,3-benzoxazol-5-yl)-1H-indol-2-yl]ethyl}carbamoyl)-7-methoxy-1,4-oxazocane-4-carboxylate (150 mg, 48.6%) as a yellow solid. LCMS (ES) [M+1]⁺ m/z: 604.

### Synthesis of (2S,7R)—N—((S)-1-cyano-2-(6-(3-methyl-2-oxo-2,3-dihydrobenzo[d]oxazol-5-yl)-1H-indol-2-yl)ethyl)-7-methoxy-1,4-oxazocane-2-carboxamide

### Synthesis of (2S,7R)—N—((R)-1-cyano-2-(6-(3-methyl-2-oxo-2,3-dihydrobenzo[d]oxazol-5-yl)-1H-indol-2-yl)ethyl)-7-methoxy-1,4-oxazocane-2-carboxamide

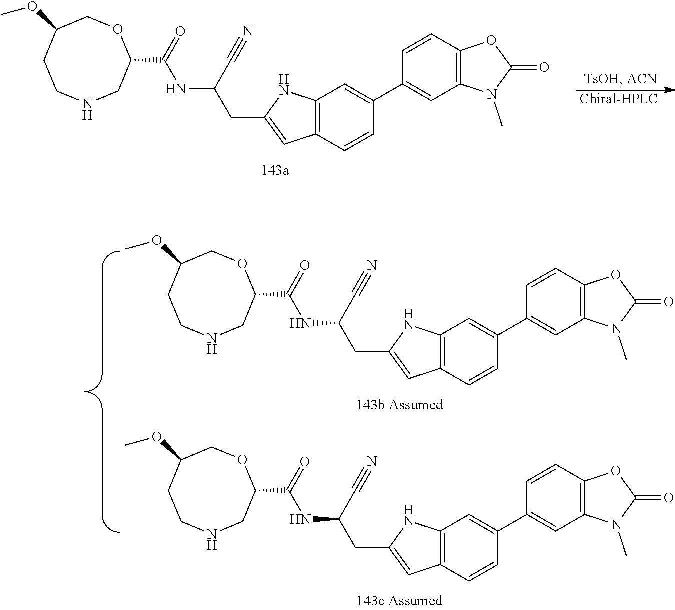

Into a 8 mL vial were added tert-butyl (2S,7R)-2-((R)-1-cyano-2-[6-(3-methyl-2-oxo-1,3-benzoxazol-5-yl)-H-indol-2-yl]ethylcarbamoyl)-7-methoxy-1,4-oxazocane-4-carboxylate (150 mg, 0.25 mmol, 1.0 equiv), CH₃CN (3 mL) and TSOH·H₂O (141 mg, 0.74 mmol, 3.0 equiv) in sequence at room temperature. The resulting mixture was stirred for 2 h at room temperature. The reaction solution was purified by reverse phase flash with the following conditions: mobile phase, MeCN in Water (0.1% NH₃·H₂O), 10% to 70% gradient in 10 min; This resulted in (2S,7R)—N-{1-cyano-2-[6-(3-methyl-2-oxo-1,3-benzoxazol-5-yl)-1H-indol-2-yl]ethyl}-7-methoxy-1,4-oxazocane-2-carboxamide (100 mg, 800%) as off-white solid. (2 S,7R)—N—{1-cyano-2-[6-(3-methyl-2-oxo-1,3-benzoxazol-5-yl)-1H-indol-2-yl]ethyl}1-7-methoxy-1,4-oxazocane-2-carboxamide (100 mg, 0.20 mmol, 1.0 equiv) was separated by Chiral-HPLC with conditions: Column: CHIRALPAK IA, 3*25 cm, 5 m; Mobile Phase A: C₀₂, Mobile Phase B: MeOH:DCM=2:1 (0.1% 2M NH₃-MeOH); Flow rate: 80 mL/min; Gradient: isocratic 45% B; Column Temperature (° C.): 35; Back Pressure (bar): 100; Wave Length: 254 nm; RT1(min): 4.45; RT2(min): 5.8; Sample Solvent: MeOH; Injection Volume: 4 mL. The fraction at 4.5 min was concentrated to afford (2S,7R)—N—((S)-1-cyano-2-(6-(3-methyl-2-oxo-2,3-dihydrobenzo[d]oxazol-5-yl)-1H-indol-2-yl)ethyl)-7-methoxy-1,4-oxazocane-2-carboxamide (22.3 mg, 22.3%) as a white solid, the fraction at 5.8 min was concentrated to afford (2S,7R)—N—((R)-1-cyano-2-(6-(3-methyl-2-oxo-2,3-dihydrobenzo[d]oxazol-5-yl)-1H-indol-2-yl)ethyl)-7-methoxy-1,4-oxazocane-2-carboxamide (24.1 mg, 24.1%) as a white solid.

143b: LCMS (ES) [M+1]⁺ m/z: 504.1

¹H NMR (400 MHz, DMSO-d₆) δ 11.14 (s, 1H), 8.74 (d, J=8.4 Hz, 1H), 7.61-7.51 (m, 3H), 7.45-7.35 (m, 2H), 7.31 (dd, J=8.2, 1.7 Hz, 1H), 6.33 (s, 1H), 5.19 (q, J=8.0 Hz, 1H), 4.11 (dd, J=11.5, 4.3 Hz, 1H), 3.96 (dd, J=9.0, 2.9 Hz, 1H), 3.60 (dd, J=11.5, 8.6 Hz, 1H), 3.43 (s, 3H), 3.41-3.33 (m, 2H), 3.31-3.27 (m, 1H), 3.26 (s, 3H), 3.09-2.89 (m, 2H), 2.50-2.37 (m, 2H), 1.95-1.89 (m, 1H), 1.66-1.53 (m, 1H).

143c:

LCMS (ES) [M+1]⁺ m/z: 504.1

¹H NMR (400 MHz, DMSO-d₆) δ 11.15 (s, 1H), 8.74 (d, J=8.2 Hz, 1H), 7.59-7.54 (m, 3H), 7.45-7.34 (m, 2H), 7.32 (d, J=8.3 Hz, 1H), 6.34 (s, 1H), 5.16 (q, J=8.1 Hz, 1H), 4.13 (dd, J=11.5, 4.1 Hz, 1H), 3.93-3.87 (m, 1H), 3.56 (dd, J=11.5, 8.5 Hz, 1H), 3.43 (s, 3H), 3.40-3.34 (m, 3H), 3.25 (s, 3H), 3.15 (dd, J=14.4, 2.8 Hz, 1H), 3.06 (dd, J=13.8, 7.9 Hz, 1H), 2.61-2.53 (m, 2H), 1.99-1.89 (m, 1H), 1.63-1.55 (m, 1H).

### Example 34. Synthesis of Compound 144a, 144b and 144c

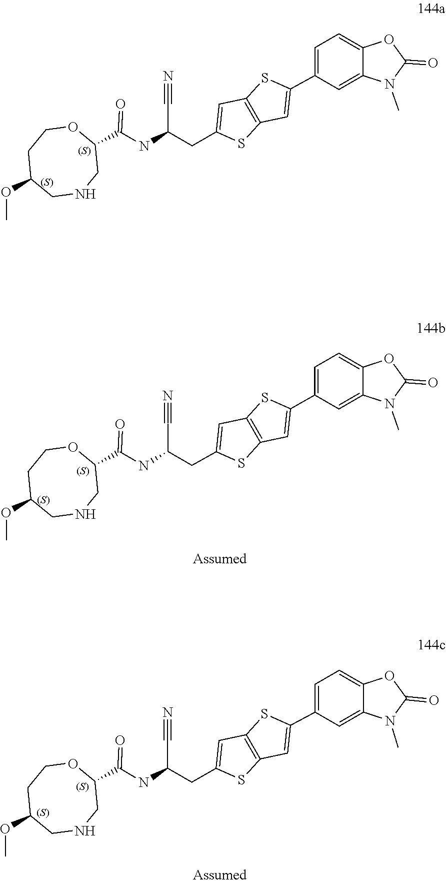

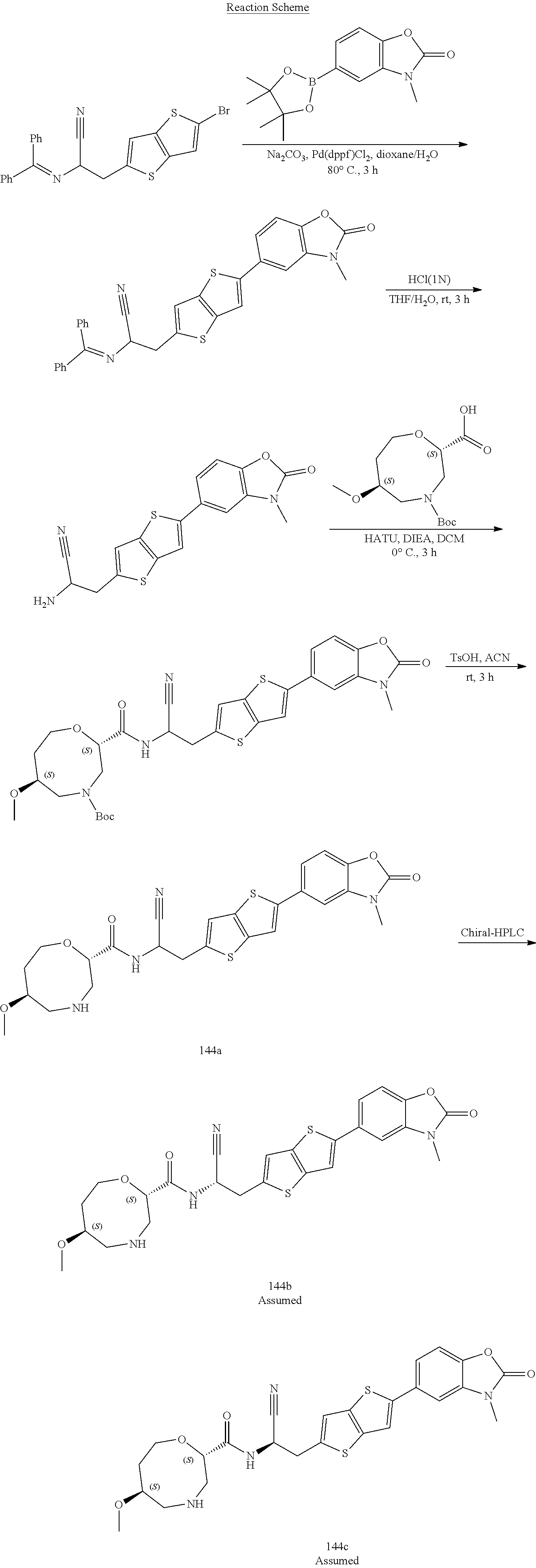

### a Synthesis of 2-[(diphenylmethylidene)amino]-3-[5-(3-methyl-2-oxo-1,3-benzoxazol-5-yl)thieno[3,2-b]thiophen-2-yl]propanenitrile

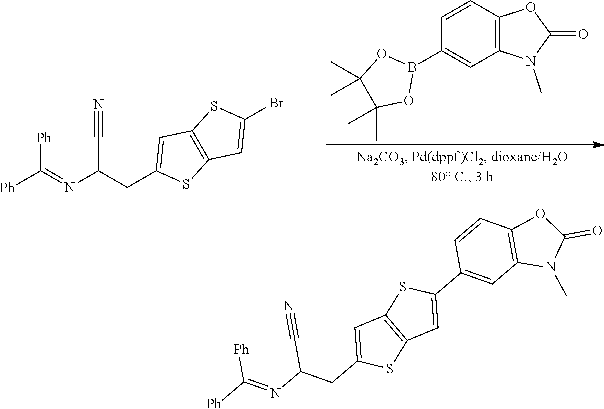 [SMILES: N#CC(Cc1cc2sc(Br)cc2s1)N=C(c1ccccc1)c1ccccc1 ; Cn1c(=O)oc2ccc(B3OC(C)(C)C(C)(C)O3)cc21 ; Cn1c(=O)oc2ccc(-c3cc4sc(CC(C#N)N=C(c5ccccc5)c5ccccc5)cc4s3)cc21]

To a solution of 3-{5-bromothieno[3,2-b]thiophen-2-yl}-2-[(diphenylmethylidene)amino]propanenitrile (500 mg, 1.11 mmol, 1.0 equiv) and 3-methyl-5-(4,4,5,5-tetramethyl-1,3,2-dioxaborolan-2-yl)-1,3-benzoxazol-2-one (304 mg, 1.11 mmol, 1.0 equiv) in 1,4-dioxane (5 mL), H₂O (0.5 mL), were added Na₂CO₃ (234 mg, 2.22 mmol, 2.0 equiv) and Pd(dppf)Cl₂·CH₂Cl₂ (90 mg, 0.11 mmol, 0.1 equiv) in sequence. The mixture was stirred for 3 h at 80° C. under nitrogen atmosphere. The reaction was cooled to room temperature, concentrated to remove the solvent, the residue was purified by silica gel column chromatography, eluted with PE/THF (2:1) to afford 2-[(diphenylmethylidene)amino]-3-[5-(3-methyl-2-oxo-1,3-benzoxazol-5-yl)thieno[3,2-b]thiophen-2-yl]propanenitrile (520 mg, 90.3%) as a yellow semi-solid. LCMS (ES, m/z): [M+H]⁺: 520.

### Synthesis of 2-amino-3-[5-(3-methyl-2-oxo-1,3-benzoxazol-5-yl)thieno[3,2-b]thiophen-2-yl]propanenitrile

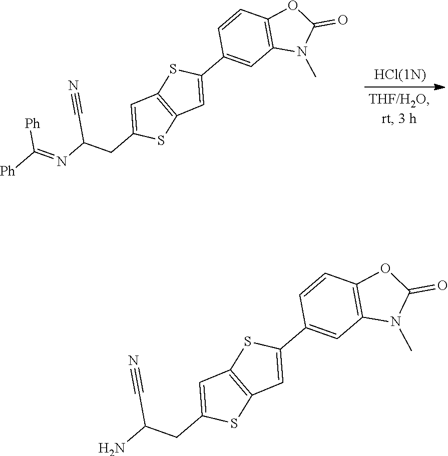 [SMILES: Cn1c(=O)oc2ccc(-c3cc4sc(CC(C#N)N=C(c5ccccc5)c5ccccc5)cc4s3)cc21 ; Cn1c(=O)oc2ccc(-c3cc4sc(CC(N)C#N)cc4s3)cc21]

Into a 100 mL round-bottom flask were added 2-[(diphenylmethylidene)amino]-3-[5-(3-methyl-2-oxo-1,3-benzoxazol-5-yl)thieno[3,2-b]thiophen-2-yl]propanenitrile (520 mg, 1.00 mmol, 1.0 equiv), THE (25 mL), H₂O (2.5 mL) and HCl (1 N) (1.2 mL) at room temperature. The resulting mixture was stirred for 3 h at room temperature. The mixture was basified to pH 9 with saturated K₂CO₃ (aq.). The resulting mixture was extracted with EtOAc (3×50 mL). The combined organic layer were washed with brine (3×50 mL), dried over anhydrous Na₂SO₄. After filtration, the filtrate was concentrated under reduced pressure. The residue was purified by silica gel column chromatography, eluted with PE/THF (1:2) to afford 2-amino-3-[5-(3-methyl-2-oxo-1,3-benzoxazol-5-yl)thieno[3,2-b]thiophen-2-yl]propanenitrile (300 mg, 84.3%) as a yellow solid. LCMS (ES, m/z): [M+H]⁺:356.

### Synthesis of tert-butyl (2S,6S)-2-({1-cyano-2-[5-(3-methyl-2-oxo-1,3-benzoxazol-5-yl)thieno[3,2-b]thiophen-2-yl]ethyl}carbamoyl)-6-methoxy-1,4-oxazocane-4-carboxylate

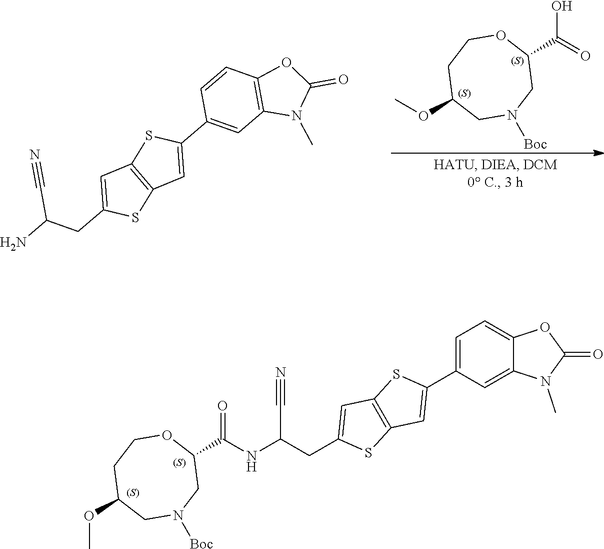

To a stirred solution of (2S,6S)-4-(tert-butoxycarbonyl)-6-methoxy-1,4-oxazocane-2-carboxylic acid (140 mg, 0.48 mmol, 1.0 equiv) and 2-amino-3-[5-(3-methyl-2-oxo-1,3-benzoxazol-5-yl)thieno[3,2-b]thiophen-2-yl]propanenitrile (171 mg, 0.48 mmol, 1.0 equiv), DIEA (187 mg, 1.45 mmol, 3.0 equiv) in DCM (3 mL) was added HATU (220 mg, 0.58 mmol, 1.2 equiv) in portions at 0° C. The resulting mixture was stirred for 3 h at 0° C. Concentrated to remove the solvent, the residue was purified by silica gel column chromatography, eluted with PE/THF (1:1) to afford tert-butyl (2S,6S)-2-({1-cyano-2-[5-(3-methyl-2-oxo-1,3-benzoxazol-5-yl)thieno[3,2-b]thiophen-2-yl]ethyl}carbamoyl)-6-methoxy-1,4-oxazocane-4-carboxylate (170 mg, 56%) as a white semi-solid. LCMS (ES, m/z): [M+H]⁺: 627.

### Synthesis of (2S,6S)—N-{1-cyano-2-[5-(3-methyl-2-oxo-1,3-benzoxazol-5-yl)thieno[3,2-b]thiophen-2-yl]ethyl}-6-methoxy-1,4-oxazocane-2-carboxamide

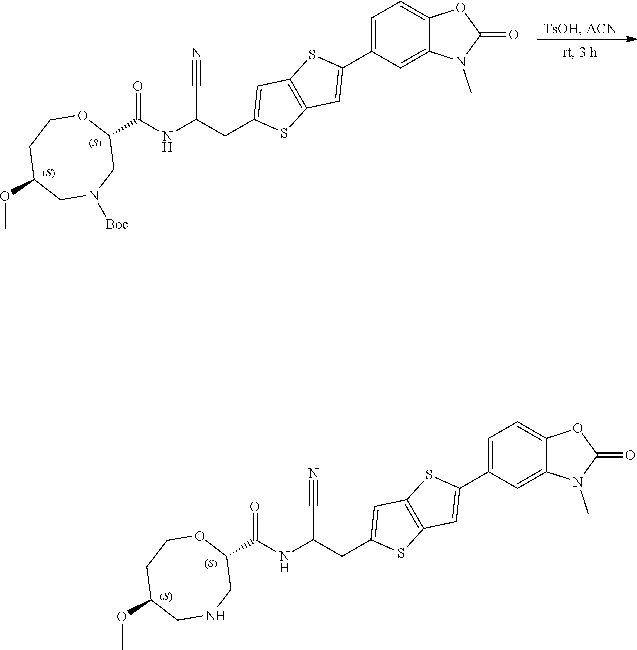

Into a 25 mL round-bottom flask were added tert-butyl (2S,6S)-2-({1-cyano-2-[5-(3-methyl-2-oxo-1,3-benzoxazol-5-yl)thieno[3,2-b]thiophen-2-yl]ethyl}carbamoyl)-6-methoxy-1,4-oxazocane-4-carboxylate (170 mg, 0.27 mmol, 1.0 equiv), ACN (5 mL) and TsOH·H₂O (154 mg, 0.81 mmol, 3.0 equiv) at room temperature. The resulting mixture was stirred for 3 h at room temperature. The reaction was purified by reversed-phase flash chromatography with the following conditions: column, C₁₈ silica gel-120 g; mobile phase, MeCN in Water (0.1% NH₃·H₂O), 10% to 50% gradient in 10 min; detector, UV 254 nm. This resulted in (2S,6S)—N-{1-cyano-2-[5-(3-methyl-2-oxo-1,3-benzoxazol-5-yl)thieno[3,2-b]thiophen-2-yl]ethyl}-6-methoxy-1,4-oxazocane-2-carboxamide (120 mg, 84%) as a white solid. LCMS (ES, m/z): [M+H]⁺: 527.

### Synthesis of (2S,6S)—N-[(1S)-1-cyano-2-[5-(3-methyl-2-oxo-1,3-benzoxazol-5-yl)thieno[3,2-b]thiophen-2-yl]ethyl]-6-methoxy-1,4-oxazocane-2-carboxamide and (2S,6S)—N-[(1R)-1-cyano-2-[5-(3-methyl-2-oxo-1,3-benzoxazol-5-yl)thieno[3,2-b]thiophen-2-yl]ethyl]-6-methoxy-1,4-oxazocane-2-carboxamide

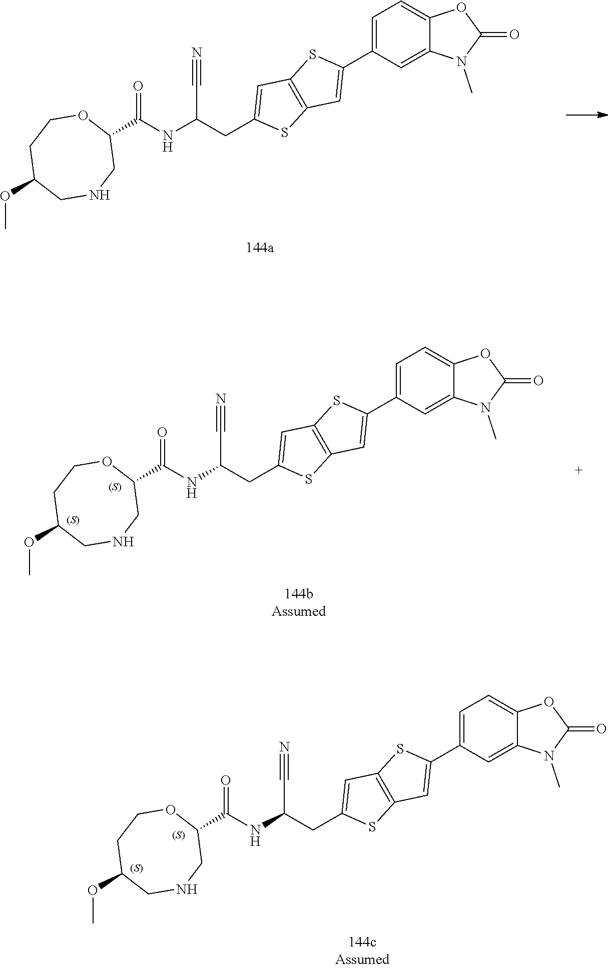

(2S,6S)—N-{1-cyano-2-[5-(3-methyl-2-oxo-1,3-benzoxazol-5-yl)thieno[3,2-b]thiophen-2-yl]ethyl}-6-methoxy-1,4-oxazocane-2-carboxamide (100 mg, 0.190 mmol, 1 equiv) was purified by Prep-HPLC with the following conditions Column: XA-CHIRALPAK IC, 2*25 cm, 5 m; Mobile Phase A: Hex:DCM=1:1—HPLC, Mobile Phase B: EtOH (0.1% DEA); Flow rate: 23 mL/min; Gradient: isocratic 50; Wave Length: 254 nm; RT1(min): 12.5; RT2(min): 17.4; Sample Solvent: EtOH:DCM=1:1—HPLC; Injection Volume: 3 mL; Number Of Runs: 5. This resulted in (2S,6S)—N-[(1S)-1-cyano-2-[5-(3-methyl-2-oxo-1,3-benzoxazol-5-yl)thieno[3,2-b]thiophen-2-yl]ethyl]-6-methoxy-1,4-oxazocane-2-carboxamide (20.5 mg, 20.50%) as a white solid and (2S,6S)—N-[(1R)-1-cyano-2-[5-(3-methyl-2-oxo-1,3-benzoxazol-5-yl)thieno[3,2-b]thiophen-2-yl]ethyl]-6-methoxy-1,4-oxazocane-2-carboxamide (20.5 mg, 20.50%) as a white solid.

### Analytical Data

LCMS (ES, m/z): [M+H]⁺: 527.0

¹H NMR (400 MHz, DMSO-d₆) δ 8.74 (d, J=8.6 Hz, 1H), 7.85 (s, 1H), 7.61 (d, J=1.7 Hz, 1H), 7.46-7.35 (m, 2H), 7.33 (s, 1H), 5.01 (td, J=8.7, 6.7 Hz, 1H), 3.95 (dd, J=10.1, 2.6 Hz, 1H), 3.88-3.78 (m, 1H), 3.75-3.66 (m, 1H), 3.56-3.41 (m, 2H), 3.40 (s, 3H), 3.28-3.22 (m, 1H), 3.21 (s, 3H), 3.20-3.13 (m, 1H), 3.03 (dd, J=13.6, 2.6 Hz, 1H), 2.70 (dd, J=14.8, 3.6 Hz, 1H), 2.29-2.14 (m, 2H), 1.64 (dd, J=14.5, 3.5 Hz, 1H).

¹H NMR (400 MHz, DMSO-d₆) δ 8.77 (d, J=8.5 Hz, 1H), 7.85 (s, 1H), 7.61 (d, J=1.8 Hz, 1H), 7.46-7.36 (m, 2H), 7.34 (s, 1H), 4.97 (td, J=8.6, 6.5 Hz, 1H), 3.91-3.80 (m, 2H), 3.68 (dt, J=12.1, 3.9 Hz, 1H), 3.56-3.40 (m, 2H), 3.40 (s, 3H), 3.20 (s, 3H), 3.29-3.14 (m, 2H), 3.13 (dd, J=13.7, 2.6 Hz, 1H), 2.71 (dd, J=14.7, 3.7 Hz, 1H), 2.36 (dd, J=13.6, 10.1 Hz, 1H), 2.22 (t, J=11.1 Hz, 1H), 1.63 (dd, J=14.4, 3.4 Hz, 1H).

### Numbered Clauses—Set 1

1. A compound of formula (I)

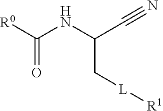

or a pharmaceutically acceptable salt or deuterated form thereof,
wherein
R⁰ is

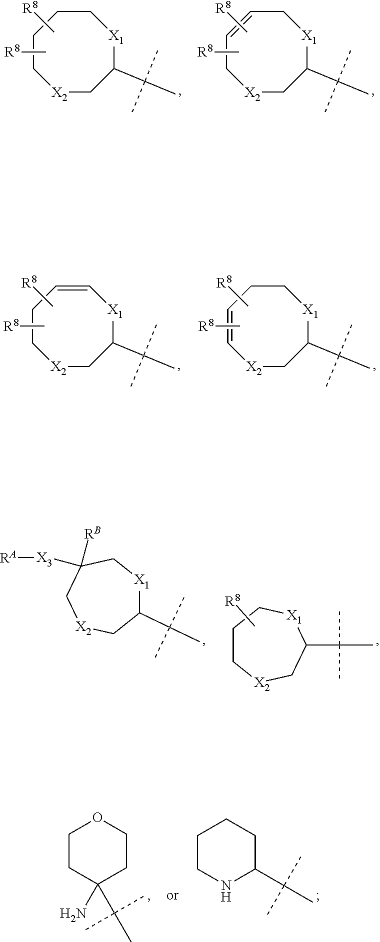

X₁ and X₂ are independently O, S, NH, N(C₁₋₆alkyl), or CR¹²R¹³, wherein at least one of X₁ and X₂ are not CR¹²R¹³;
X₃ is O, S, NH, or N(C₁₋₆alkyl);
R^{A} is H, C₁₋₆alkyl, C₁₋₆alkylene-carbocyclyl, or C₁₋₆alkylene-heteroaryl; and
R^{B} is C₁₋₆alkyl, C₂₋₆alkenyl, C₁₋₆alkylene-carbocyclyl, or C₁₋₆alkylene-heteroaryl; or
R^{A} and R^{B} are taken together to form a heterocyclyl;
L is polycyclic cycloalkylene or polycyclic heteroarylene, wherein a first atom of a first ring of the polycyclic heteroarylene is connected to

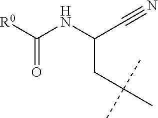

and a second atom of a second ring of the polycyclic heteroarylene is connected to R¹;
wherein L is independently substituted by 0-4 R¹⁰;
R¹ is

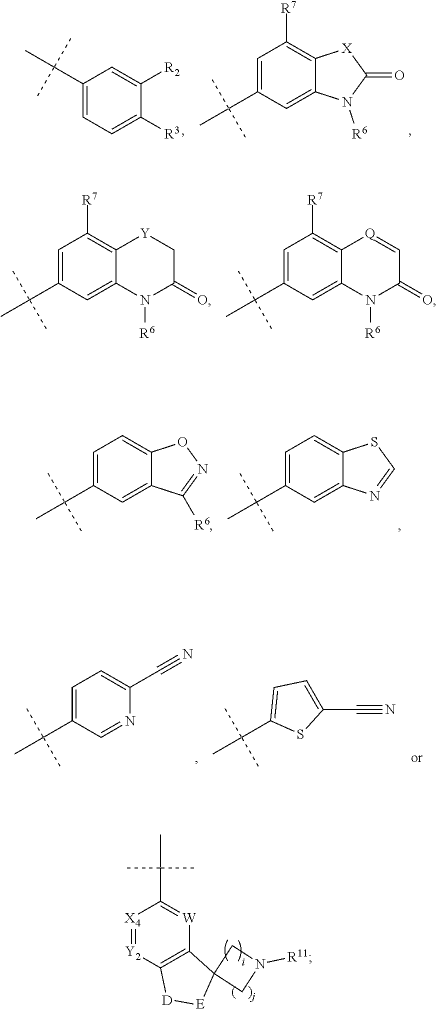

R² is H, F, Cl, Br, OSO₂C₁₋₆alkyl, or C₁₋₆alkyl;
R³ is H, F, Cl, Br, CN, C₁₋₆haloalkyl, SO₂C₁₋₆alkyl, CONH₂ or SO₂NR⁴R⁵, wherein R⁴ and R⁵ together with the nitrogen atom to which they are attached form a heterocyclyl;
X is O, S, CHF, or CF₂;
Y is O or S;
Q is CH or N;
R⁶ is C₁₋₆alkyl optionally substituted by 1, 2 or 3 F, or by OH, O—C₁₋₆alkyl, N(C₁₋₆alkyl)₂, cycloalkyl, or heterocyclyl;
R⁷ is H, F, Cl, Br, or C₁₋₆alkyl;
R⁸ and R¹² are independently H, OH, halogen, NH₂, COOH, C₁₋₆alkyl, C₁₋₆ alkyl-OH, C₁₋₆alkoxy or halogenated C₁₋₆alkoxy;
each R¹³ is independently H, F, Cl, Br, I or C₁-C₆ alkyl;
each R¹⁰ is independently oxo, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH;
W, X₄ and Y₂ are each independently CH or N, provided that a maximum of one of W, X₄ and Y₂ can be N;
D-E is N(H)—C(O), N(C₁₋₆alkyl)-C(O), CH₂CH₂, C(O)—O or CH₂—O;
R¹¹ is H, C₁₋₆alkyl, alkylene-O-alkyl, or heterocyclyl; and
i and j are each independently 1, 2 or 3; provided that the sum of i+j is 2, 3 or 4.

2. The compound of clause 1, or a pharmaceutically acceptable salt or deuterated form thereof, wherein L is a polycyclic cycloalkylene.

3. The compound of clause 2, or a pharmaceutically acceptable salt or deuterated form thereof, wherein the polycyclic cycloalkylene comprises 4-10 carbon atoms.

4. The compound of any one of clauses 1-3, or a pharmaceutically acceptable salt or deuterated form thereof, wherein the polycyclic cycloalkylene is connected to R¹ at a first quaternary carbon of the polycyclic cycloalkylene and independently connected to

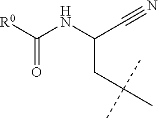

through a second quaternary carbon of the polycyclic cycloalkylene.

5. The compound of any one of clauses 1-4, or a pharmaceutically acceptable salt or deuterated form thereof, wherein the polycyclic cycloalkylene is

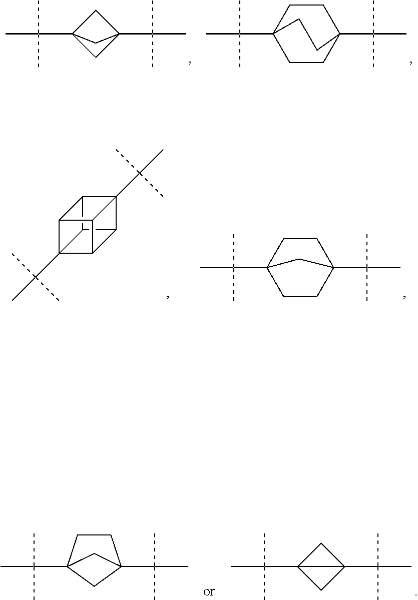

6. The compound of any one of clauses 1-5, or a pharmaceutically acceptable salt or deuterated form thereof, wherein the polycyclic cycloalkylene is

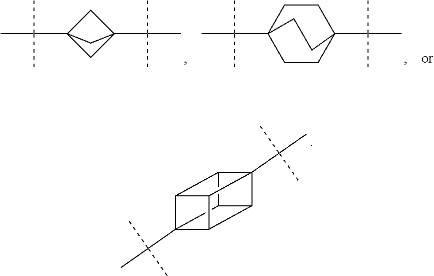

7. The compound of clause 1, or a pharmaceutically acceptable salt or deuterated form thereof, wherein L is a polycyclic heteroarylene substituted by 0-4 R¹⁰.

8. The compound of clause 7, or a pharmaceutically acceptable salt or deuterated form thereof, wherein the polycyclic heteroarylene is

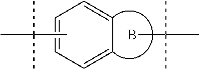

substituted by 0-4 R¹⁰, wherein ring B is a heteroaryl ring.

9. The compound of clause 7 or 8, or a pharmaceutically acceptable salt or deuterated form thereof, wherein the polycyclic heteroarylene comprises 8-12 ring atoms and is substituted by 0-4 R¹⁰.

10. The compound of any one of clauses 7-9, or a pharmaceutically acceptable salt or deuterated form thereof, wherein the polycyclic heteroarylene comprises 9-10 ring atoms and is substituted by 0-4 R¹⁰.

11. The compound of any one of clauses 7-10, or a pharmaceutically acceptable salt or deuterated form thereof, wherein the polycyclic heteroarylene comprises 9 ring atoms and 1 or 2 heteroatoms selected from O, N, or S, and the polycyclic heteroarylene is substituted by 0-4 R¹⁰.

12. The compound of any one of clauses 7-11, or a pharmaceutically acceptable salt or deuterated form thereof, wherein the polycyclic heteroarylene is benzothienylene substituted by 0-4 R¹⁰.

13. The compound of any one of clauses 8-12, or a pharmaceutically acceptable salt or deuterated form thereof, wherein the polycyclic heteroarylene is

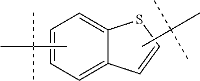

substituted by 0-4 R¹⁰.

14. The compound of clause 13, or a pharmaceutically acceptable salt or deuterated form thereof, wherein

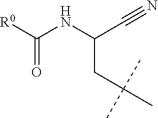

is attached to the phenyl ring of

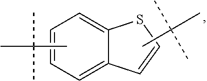

and R¹ is attached the thienyl ring of

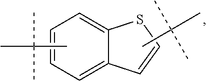

wherein

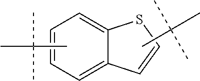

is substituted by 0-4 R¹⁰.

15. The compound of clause 13, or a pharmaceutically acceptable salt or deuterated form thereof, wherein

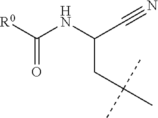

is attached to the thienyl ring of

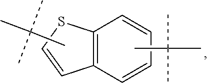

and R¹ is attached to the phenyl ring of

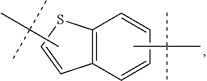

and wherein

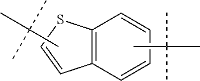

is substituted by 0-4 R¹⁰.

16. The compound of any one of clauses 8-15, or a pharmaceutically acceptable salt or deuterated form thereof, wherein the polycyclic heteroarylene is

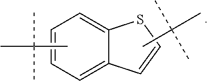

17. The compound of any one of clauses 8-16, or a pharmaceutically acceptable salt or deuterated form thereof, wherein the polycyclic heteroarylene is

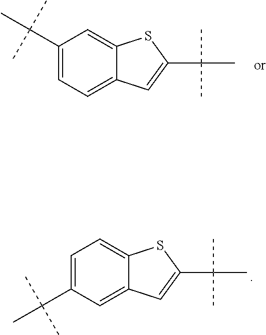

18. The compound of any one of clauses 8-16, or a pharmaceutically acceptable salt or deuterated form thereof, wherein the polycyclic heteroarylene is

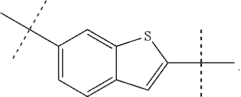

19. The compound of any one of clauses 8-15, or a pharmaceutically acceptable salt or deuterated form thereof, wherein L is

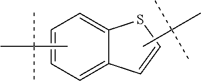

substituted by 1-4 R¹⁰.

20. The compound of any one of clauses 8-15 or 19, or a pharmaceutically acceptable salt or deuterated form thereof, wherein L is

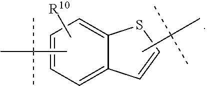

21. The compound of any one of clauses 8-15, 19, or 20, or a pharmaceutically acceptable salt or deuterated form thereof, wherein R¹⁰ is halo.

22. The compound of any one of clauses 8-15 or 19-21, or a pharmaceutically acceptable salt or deuterated form thereof, wherein

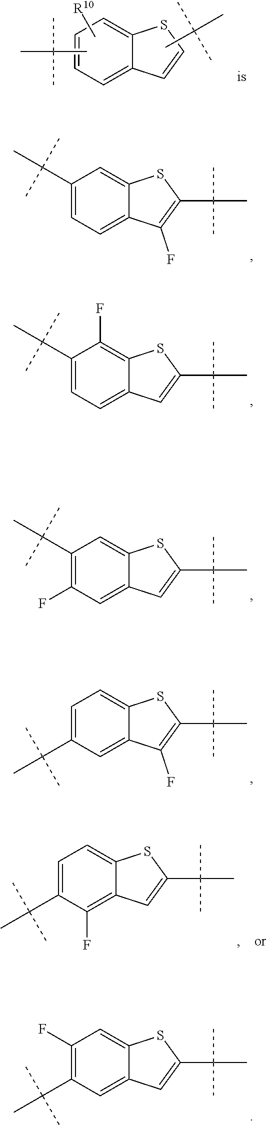

23. The compound of clause 1, wherein the compound is of Formula (II),

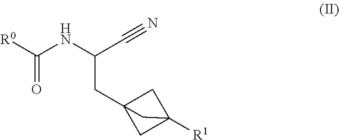

or a pharmaceutically acceptable salt or deuterated form thereof.

24. The compound of clause 1, wherein the compound is of Formula (III),

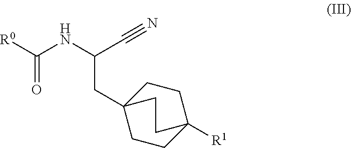

or a pharmaceutically acceptable salt or deuterated form thereof.

25. The compound of clause 1, wherein the compound is of Formula (IV),

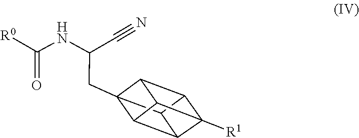

or a pharmaceutically acceptable salt or deuterated form thereof.

26. The compound of clause 1, wherein the compound is of Formula (V),

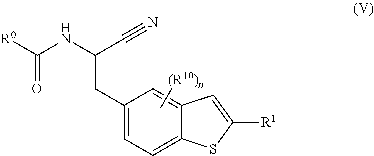

or a pharmaceutically acceptable salt or deuterated form thereof, wherein n is 0 or 1.

27. The compound of clause 1, wherein the compound is of Formula (VI),

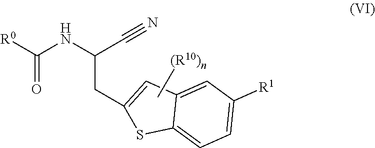

or a pharmaceutically acceptable salt or deuterated form thereof, wherein n is 0 or 1.

28. The compound of clause 1, wherein the compound is of Formula (VII),

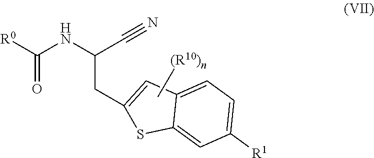

or a pharmaceutically acceptable salt or deuterated form thereof, wherein n is 0 or 1.

29. The compound of anyone clauses 26-28, or a pharmaceutically acceptable salt or deuterated form thereof, wherein n is 0.

30. The compound of any one of clauses 26-28, or a pharmaceutically acceptable salt or deuterated form thereof, wherein n is 1 and R¹⁰ is halo.

31. The compound of any one of clauses 26-28 or 30, or a pharmaceutically acceptable salt or deuterated form thereof, wherein n is 1 and R¹⁰ is F.

32. The compound of any one of clauses 1-31, or a pharmaceutically acceptable salt or deuterated form thereof, wherein
R¹ is

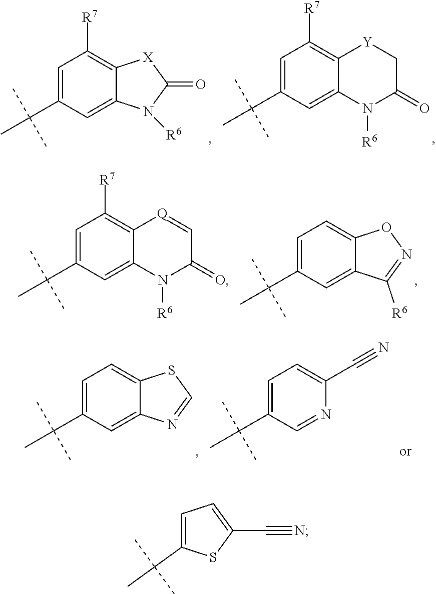

X is O, S or CF₂;
Y is O or S;
Q is CH or N;
R⁶ is C₁₋₃alkyl, wherein said C₁₋₃alkyl is optionally substituted by 1, 2 or 3 F, OH, OC₁₋₃alkyl, N(C₁₋₃alkyl)₂, cyclopropyl, or tetrahydropyran; and
R⁷ is H, F, C₁ or CH₃.

33. The compound of any one of clauses 1-32, or a pharmaceutically acceptable salt or deuterated form thereof, wherein
R¹ is

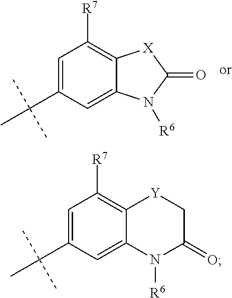

X is O, S or CF₂;
Y is O or S;
R⁶ is C₁₋₃alkyl, wherein said C₁₋₃alkyl is optionally substituted by 1, 2 or 3 F, OH, OC₁₋₃alkyl, N(C₁₋₃alkyl)₂, cyclopropyl, or tetrahydropyran; and
R⁷ is H, F, C₁ or CH₃.

34. The compound of clause 1-33, or a pharmaceutically acceptable salt or deuterated form thereof, wherein R¹ is

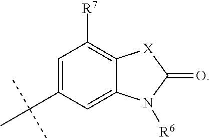

35. The compound of clause 34, or a pharmaceutically acceptable salt or deuterated form thereof, wherein, X is O; R⁶ is C₁₋₃alkyl; and R⁷ is H.

36. The compound of any one of clauses 1-34, or a pharmaceutically acceptable salt or deuterated form thereof, wherein
R¹ is

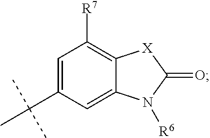

X is O;
R⁶ is C₁₋₃alkyl, wherein said C₁₋₃alkyl is optionally substituted by 1, 2 or 3 F; and
R⁷ is H.

37. The compound of any one of clauses 1-34, or a pharmaceutically acceptable salt or deuterated form thereof, wherein, R¹ is

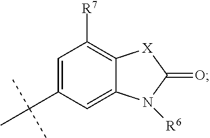

X is O;
R⁶ is C₁₋₃alkyl; and
R⁷ is H.

38. The compound of any one of clauses 1-33, or a pharmaceutically acceptable salt thereof, wherein R¹ is

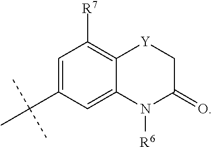

39. The compound of any one of clauses 1-32, or a pharmaceutically acceptable salt or deuterated form thereof, wherein R¹ is

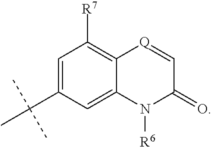

40. The compound of any one of clauses 1-32, or a pharmaceutically acceptable salt or deuterated form thereof, wherein R¹ is

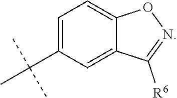

41. The compound of any one of clauses 32-40, or a pharmaceutically acceptable salt or deuterated form thereof, wherein R⁶ is methyl.

42. The compound of any one of clauses 32-40, or a pharmaceutically acceptable salt or deuterated form thereof, wherein R⁶ is ethyl.

43. The compound of any one of clauses 32-40, or a pharmaceutically acceptable salt or deuterated form thereof, wherein R⁶ is propyl.

44. The compound of any one of clauses 1-32, or a pharmaceutically acceptable salt or deuterated form thereof, wherein R¹ is

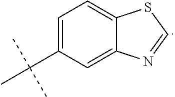

45. The compound of any one of clauses 1-32, or a pharmaceutically acceptable salt or deuterated form thereof, wherein R¹ is

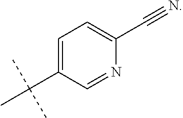

46. The compound of any one of clauses 1-32, or a pharmaceutically acceptable salt or deuterated form thereof, wherein R¹ is

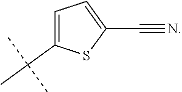

47. The compound of any one of clauses 1-6, wherein the compound is of Formula (VIII):

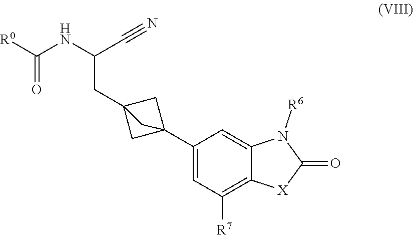

or a pharmaceutically acceptable salt or deuterated form thereof.

48. The compound of any one of clauses 1-6, wherein the compound is of Formula (IX),

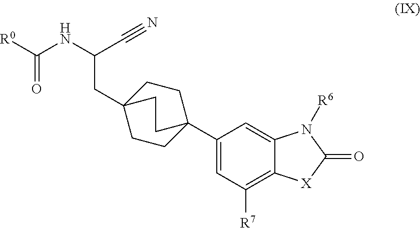

or a pharmaceutically acceptable salt or deuterated form thereof.

49. The compound of any one of clauses 1-6, wherein the compound is of Formula (X),

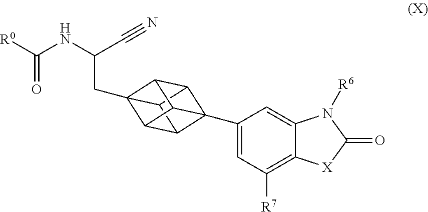

or a pharmaceutically acceptable salt or deuterated form thereof.

50. The compound of clause 1, wherein the compound is of Formula (XI),

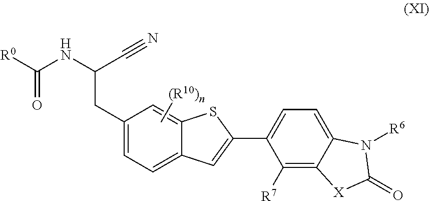

or a pharmaceutically acceptable salt or deuterated form thereof, wherein
n is 0 or 1.

51. The compound of clause 1, wherein the compound is of Formula (XII),

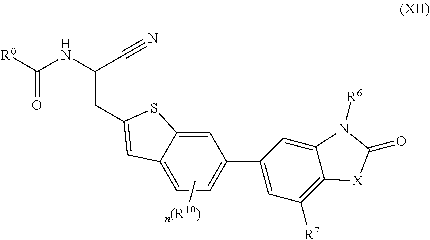

or a pharmaceutically acceptable salt or deuterated form thereof, wherein
n is 0 or 1

52. The compound of clause 1, wherein the compound is of Formula (XIII),

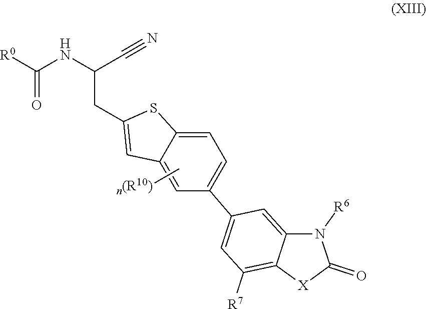

or a pharmaceutically acceptable salt or deuterated form thereof, wherein
n is 0 or 1

53. The compound of any one of clauses 50-52, or a pharmaceutically acceptable salt or deuterated form thereof, wherein n is 0.

54. The compound of any one of clauses 50-52, or a pharmaceutically acceptable salt or deuterated form thereof, wherein n is 1 and R¹⁰ is halo.

55. The compound of any one of clauses 50-52 or 54, or a pharmaceutically acceptable salt or deuterated form thereof, wherein n is 1 and R¹⁰ is F.

56. The compound of any one of clauses 50-55, or a pharmaceutically acceptable salt or deuterated form thereof, wherein X is O.

57. The compound of any one of clauses 50-56, or a pharmaceutically acceptable salt or deuterated form thereof, wherein R⁶ is C₁₋₃ alkyl.

58. The compound of any one of clauses 50-57, or a pharmaceutically acceptable salt or deuterated form thereof, wherein R⁷ is H.

59. The compound of any one of clauses 50-57, or a pharmaceutically acceptable salt or deuterated form thereof, wherein X is O, R⁶ is C₁₋₃ alkyl and R⁷ is H.

60. The compound of any one of clauses 1-59, or a pharmaceutically acceptable salt or deuterated form thereof, wherein R⁰ is

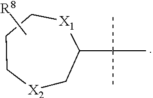

61. The compound of any one of clauses 1-60, or a pharmaceutically acceptable salt or deuterated form thereof, wherein R⁰ is

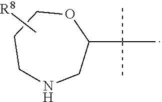

62. The compound of any one of clauses 60-61, wherein R⁸ is H.

63. The compound of any one of clauses 1-59, or a pharmaceutically acceptable salt or deuterated form thereof, wherein R⁰ is

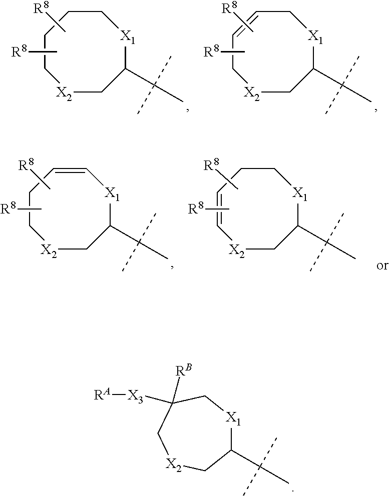

64. The compound of any one of clauses 1 or 63, or a pharmaceutically acceptable salt or deuterated form thereof, wherein R⁰ is

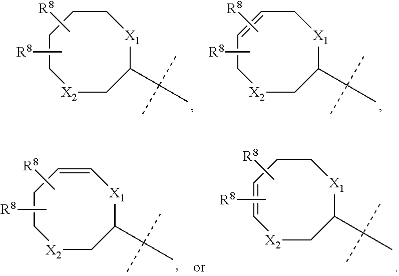

65. The compound of clause 64, or a pharmaceutically acceptable salt or deuterated form thereof, wherein R⁰ is

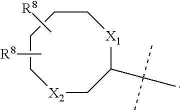

66. The compound of clause 64, or a pharmaceutically acceptable salt or deuterated form thereof, wherein R⁰ is

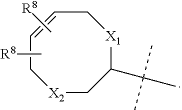

67. The compound of clause 64, or a pharmaceutically acceptable salt or deuterated form thereof, wherein R⁰ is

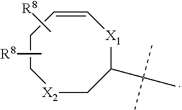

68. The compound of clause 64, or a pharmaceutically acceptable salt or deuterated form threof, wherein R⁰ is

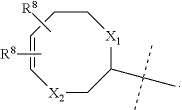

69. The compound of any one of clauses 1 or 63-68, or a pharmaceutically acceptable salt or deuterated form thereof, wherein at least one R⁸ is H.

70. The compound of any one of clauses 1 or 63-68, or a pharmaceutically acceptable salt or deuterated form thereof, wherein at least one R⁸ is methoxy.

71. The compound of any one of clauses 1 or 63-68, or a pharmaceutically acceptable salt or deuterated form thereof, wherein at least one R⁸ is ethoxy.

72. The compound of any one of clauses 1 or 63-68, or a pharmaceutically acceptable salt or deuterated form thereof, wherein at least one R⁸ is OH.

73. The compound of any one of clauses 1-62, or a pharmaceutically acceptable salt or deuterated form thereof, wherein R⁰ is

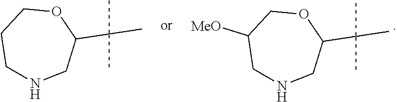

74. The compound of any one of clauses 1-62 or 73, or a pharmaceutically acceptable salt or deuterated form thereof, wherein R⁰ is

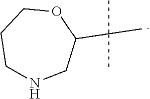

75. The compound of any one of clauses 1-62 or 73, or a pharmaceutically acceptable salt or deuterated form thereof, wherein R⁰ is

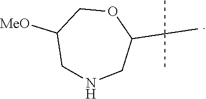

76. The compound of clause 63 or 64, or a pharmaceutically acceptable salt or deuterated thereof, wherein R⁰ is

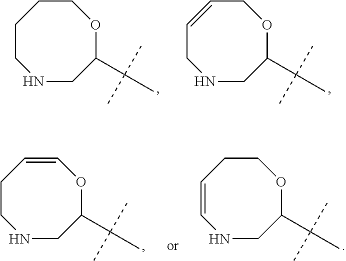

77. The compound of clause 76, or a pharmaceutically acceptable salt or deuterated form thereof, wherein R⁰ is

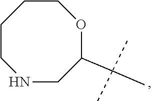

78. The compound of clause 76, or a pharmaceutically acceptable salt or deuterated form thereof, wherein R⁰ is

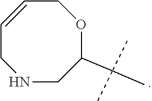

79. The compound of clause 76, or a pharmaceutically acceptable salt or deuterated form thereof, wherein R⁰ is

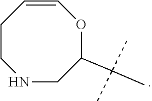

80. The compound of clause 76, or a pharmaceutically acceptable salt or deuterated form thereof, wherein R⁰ is

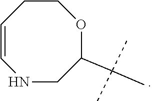

81. The compound of any one of clauses 1-59 or 63, or a pharmaceutically acceptable salt or deuterated form thereof, wherein R⁰ is

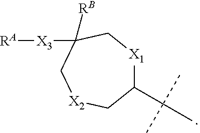

82. The compound of clause 81, or a pharmaceutically acceptable salt or deuterated form thereof, wherein R⁰ is

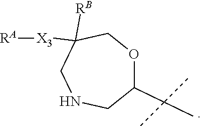

83. The compound of any one of clauses 62 or 81-82, or a pharmaceutically acceptable salt or deuterated form thereof, wherein R^{A} is H or C₁₋₆alkyl.

84. The compound of clause 83, or a pharmaceutically acceptable salt or deuterated form thereof, wherein R^{A} is H.

85. The compound of clause 83, or a pharmaceutically acceptable salt or deuterated form thereof, wherein R^{A} is methyl.

86. The compound of any one of clauses 63 or 81-85, or a pharmaceutically acceptable salt or deuterated form thereof, wherein R^{B} is C₁₋₆alkyl, C₂₋₆ alkenyl, C₁₋₆alkylene-carbocyclyl, or C₁₋₆alkylene-heteroaryl.

87. The compound of clause 86, or a pharmaceutically acceptable salt or deuterated form thereof, wherein R^{B} is C₁₋₆alkyl, C₁₋₆alkylene-aryl or —C₁₋₆alkylene-5-6 membered heteroaryl.

88. The compound of clause 81 or 82, or a pharmaceutically acceptable salt or deuterated form thereof, wherein R⁰ is

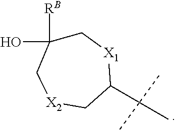

89. The compound of clause 82 or 83, or a pharmaceutically acceptable salt or deuterated form thereof, wherein R⁰ is

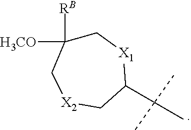

90. The compound of clause 82 or 83, or a pharmaceutically acceptable salt or deuterated form thereof, wherein R⁰ is

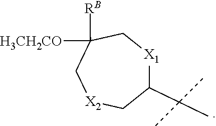

91. The compound of any one of clauses 86-90, or a pharmaceutically acceptable salt or deuterated form thereof, wherein R^{B} is methyl.

92. The compound of any one of clauses 86-90, or a pharmaceutically acceptable salt or deuterated form thereof, wherein R^{B} is ethyl.

93. The compound of any one of clauses 88-90, wherein R^{B} is H.

94. The compound of clause 89, or a pharmaceutically acceptable salt or deuterated form thereof, wherein R⁰ is

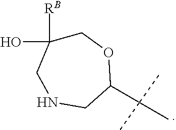

95. The compound of clause 90, or a pharmaceutically acceptable salt or deuterated form thereof, wherein R⁰ is

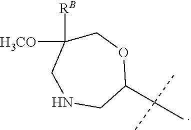

96. The compound of clause 91, or a pharmaceutically acceptable salt or deuterated form thereof, wherein R⁰ is

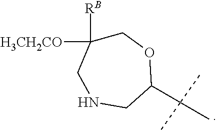

97. The compound of any one of clauses 94-96, or a pharmaceutically acceptable salt or deuterated form thereof, wherein R^{B} is methyl.

98. The compound of any one of clauses 94-96, or a pharmaceutically acceptable salt or deuterated form thereof, wherein R^{B} is ethyl.

99. The compound of any one of clauses 81-87, or a pharmaceutically acceptable salt or deuterated form thereof, wherein X₃ is O.

100. The compound of any one of clauses 1-59, or a pharmaceutically acceptable salt or deuterated form thereof, wherein R⁰ is

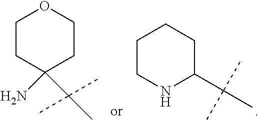

101. The compound of any one of clauses 47-100, or a pharmaceutically acceptable salt or deuterated form thereof, wherein R⁶ is C₁₋₃ alkyl.

102. The compound of clause 101, or a pharmaceutically acceptable salt or deuterated form thereof, wherein R⁶ is CH₃.

103. The compound of any one of clauses 47-100, or a pharmaceutically acceptable salt or deuterated form thereof, wherein R⁷ is H.

104. The compound of any one of clauses 47-103, wherein R¹⁰ is —H or —F.

105. The compound of clause 1, or a pharmaceutically acceptable salt or deuterated form thereof, wherein the compound is selected from Table 1.

106. A pharmaceutical composition comprising an effective amount of a compound of any one of clauses 1-105, or a pharmaceutically acceptable salt or deuterated form thereof and a pharmaceutically acceptable adjuvant, diluent or carrier.

107. A method for treating an obstructive disease of the airway in a patient in need thereof, comprising, administering to the patient an effective amount of a compound of any one of clauses 1-105, or the composition of clause 106.

108. The method of clause 107, wherein the obstructive disease of the airway is asthma, chronic obstructive pulmonary disease (COPD), bronchitis, emphysema, cystic fibrosis (CF), bronchiectasis, sarcoidosis, alpha-1 antitrypsin (A1AT) deficiency, farmer's lung and related diseases, hypersensitivity pneumonitis, lung fibrosis, complications of lung transplantation, vasculitic and thrombotic disorders of the lung vasculature, pulmonary hypertension, antitussive activity including treatment of chronic cough associated with inflammatory and secretory conditions of the airways, iatrogenic cough, acute and chronic rhinitis including rhinitis medicamentosa, and vasomotor rhinitis; perennial and seasonal allergic rhinitis including rhinitis nervosa (hay fever), nasal polyposis; acute viral infection including the common cold, and infection due to a respiratory virus, acute lung injury, or acute respiratory distress syndrome (ARDS).

109. The method of clause 108, wherein the obstructive disease of the airway is asthma.

110. The method of clause 108, wherein the obstructive disease of the airway is acute respiratory distress syndrome (ARDS).

111. The method of clause 108, wherein the obstructive disease of the airway is bronchitis.

112. The method of clause 108, wherein the obstructive disease of the airway is lung fibrosis.

113. The method of clause 108, wherein the obstructive disease of the airway is emphysema.

114. The method of clause 108, wherein the obstructive disease of the airway is cystic fibrosis (CF).

115. The method of clause 108, wherein the obstructive disease of the airway is bronchiectasis.

116. The method of clause 108, wherein the obstructive disease of the airway is sarcoidosis.

117. The method of clause 108, wherein the obstructive disease of the airway is alpha-1 antitrypsin (A1AT) deficiency.

118. The method of clause 108, wherein the obstructive disease of the airway is farmer's lung.

119. The method of clause 108, wherein the obstructive disease of the airway is hypersensitivity pneumonitis.

120. The method of clause 108, wherein the obstructive disease of the airway is a complication of lung transplantation.

121. The method of clause 108, wherein the obstructive disease of the airway is a vasculitic or thrombotic disorder of the lung vasculature.

122. The method of clause 108, wherein the obstructive disease of the airway is pulmonary hypertension.

123. The method of clause 108, wherein the obstructive disease of the airway is iatrogenic cough.

124. The method of clause 108, wherein the obstructive disease of the airway is acute rhinitis.

125. The method of clause 108, wherein the obstructive disease of the airway is chronic rhinitis.

126. The method of clause 108, wherein the obstructive disease of the airway is rhinitis medicamentosa or vasomotor rhinitis.

127. The method of clause 108, wherein the obstructive disease of the airway is nasal polyposis.

128. The method of clause 108, wherein the obstructive disease of the airway is COPD.

129. The method of clause 109, wherein the asthma is bronchial, allergic, intrinsic, extrinsic, exercise-induced or drug-induced asthma.

130. The method of clause 111, wherein the bronchitis is infectious bronchitis or eosinophilic bronchitis.

131. The method of clause 112, wherein the lung fibrosis is idiopathic pulmonary fibrosis, cryptogenic fibrosing alveolitis, idiopathic interstitial pneumonia, or fibrosis complicating anti-neoplastic therapy or chronic infection.

132. The method of clause 113, wherein the bronchiectasis is non-cystic fibrosis bronchiectasis (NCFBE).

133. The method of clause 113, wherein the bronchiectasis is associated with cystic fibrosis.

134. The method of clause 119, wherein the pulmonary hypertension is pulmonary arterial hypertension.

135. A method for treating cystic fibrosis in a patient in need thereof, comprising, administering to the patient an effective amount of a compound of any one of clauses 1-105, or the composition of clause 106.

136. The method of clause 135, wherein the treating comprises improving the lung function of the patient, as compared to the lung function of the patient prior to treatment.

137. The method of clause 135, wherein improving lung function of the patient comprises increasing the patient's forced expiratory volume in 1 second (FEV₁), increasing the patient's forced vital capacity (FVC), increasing the patient's peak expiratory flow rate (PEFR), or increasing the patient's forced expiratory flow between 25% and 75% of FVC (FEF_{(25-75%)}), as compared to the respective value for the patient prior treatment.

138. The method of clause 135 or 137, wherein the lung function is measured by spirometry.

139. A method for treating bronchiectasis in a patient in need thereof, comprising, administering to the patient, an effective amount of a compound of any one of clauses 1-105, or the composition of clause 106.

140. The method of clause 139, wherein the bronchiectasis is non-cystic fibrosis bronchiectasis (NCFBE).

141. The method of clause 139, wherein the bronchiectasis is associated with cystic fibrosis.

142. The method of any one of clauses 139-141, wherein treating comprises improving the lung function of the patient, as compared to the lung function of the patient prior to treatment.

143. The method of clause 142, wherein improving lung function of the patient comprises increasing the patient's forced expiratory volume in 1 second (FEV₁), increasing the patient's forced vital capacity (FVC), increasing the patient's peak expiratory flow rate (PEFR), or increasing the patient's forced expiratory flow between 25% and 75% of FVC (FEF_{(25-75%)}), as compared to the respective value for the patient prior to treatment.

144. The method of clause 142 or 143, wherein the lung function is measured by spirometry.

145. The method of any one of clauses 139-144, wherein treating comprises decreasing the rate of pulmonary exacerbation, as compared to the rate of pulmonary exacerbation of the patient prior to treatment.

146. The method of any one of clauses 139-145, wherein treating comprises increasing the time to first pulmonary exacerbation, as compared to an untreated patient.

147. The method of clause 144 or 145, wherein the pulmonary exacerbation is characterized by three or more of the following symptoms exhibited for at least 48 hours by the patient: (1) increased cough; (2) increased sputum volume or change in sputum consistency; (3) increased sputum purulence; (4) increased breathlessness and/or decreased exercise tolerance; (5) fatigue and/or malaise; (6) hemoptysis.

148. A method for treating chronic rhinosinusitis (CRS) in a patient in need thereof, comprising, administering to the patient, an effective amount of a compound of any one of clauses 1-105, or the composition of clause 106.

149. The method of clause 108, wherein the chronic rhinosinusitis is chronic rhinosinusitis without nasal polyps (CRSsNP).

150. The method of clause 148, wherein the chronic rhinosinusitis is chronic rhinosinusitis with nasal polyps (CRSwNP).

151. The method of any one of clauses 148-150, wherein the chronic rhinosinusitis is refractory chronic rhinosinusitis.

152. The method of any one of clauses 148-150, wherein treating comprises reducing, diminishing the severity of, delaying the onset of, or eliminating one or more symptoms of CRS.

153. The method of clause 152, wherein the one or more symptoms of CRS is nasal congestion; nasal obstruction; nasal discharge; post-nasal drip; facial pressure; facial pain; facial fullness; reduced smell; depression; mucosal edema; mucopurulent discharge; obstruction of the middle meatus; mucosal changes within the ostiomeatal complex and sinuses; or rhinorrhea.

154. A method for treating hidradenitis suppurativa (HS) in a patient in need thereof, comprising, administering to the patient, an effective amount of a compound of any one of clauses 1-105, or the composition of clause 106.

155. The method of clause 154, wherein the hidradenitis suppurativa (HS) is Hurley stage I.

156. The method of clause 154, wherein the hidradenitis suppurativa (HS) is Hurley stage II.

157. The method of clause 154, wherein the hidradenitis suppurativa (HS) is Hurley stage III.

158. A method for treating cancer in a patient in need thereof, comprising, administering to the patient, an effective amount of a compound of any one of clauses 1-105, or the composition of clause 106.

159. The method of clause 158, wherein the cancer is a metastatic cancer.

160. The method of clause 158, wherein the metastatic cancer is breast to lung metastatic cancer.

161. The method of clause 159, wherein the metastatic cancer comprises metastasis of breast cancer to the brain, bone, pancreas, lymph nodes or liver.

162. The method of clause 159, wherein the metastatic cancer comprises metastasis of bone cancer to the lung.

163. The method of clause 159, wherein the metastatic cancer comprises metastasis of colorectal cancer to the peritoneum, the pancreas, the stomach, the lung, the liver, the kidney, or the spleen.

164. The method of clause 159, wherein the metastatic cancer comprises metastasis of stomach cancer to the mesentery, the spleen, the pancreas, the lung, the liver, the adrenal gland, or the ovary.

165. The method of clause 159, wherein the metastatic cancer comprises metastasis of liver cancer to the intestine, spleen, pancreas, stomach, lung, or the kidney.

166. The method of clause 159, wherein the metastatic cancer comprises metastasis of lymphoma to the kidney, ovary, liver, bladder, or the spleen.

167. A method for treating lupus nephritis in a patient in need thereof, comprising, administering to the patient, an effective amount of a compound of any one of clauses 1-105, or the composition of clause 106.

168. A method for treating rheumatoid arthritis in a patient in need thereof, comprising, administering to the patient, an effective amount of a compound of any one of clauses 1-105, or the composition of clause 106.

169. A method for treating inflammatory bowel disease (IBD) in a patient in need thereof, comprising, administering to the patient, an effective amount of a compound of any one of clauses 1-105, or the composition of clause 106.

170. The method of clause 169, wherein the inflammatory bowel disease (IBD) is Crohn's disease.

171. The method of clause 169, wherein the inflammatory bowel disease (IBD) is ulcerative colitis.

172. A method for treating an anti-neutrophil cytoplasmic antibody (ANCA) associated vasculitis in a patient in need thereof, comprising, administering to the patient, an effective amount of a compound of any one of clauses 1-105, or the composition of clause 106, wherein the ANCA associated disease is granulomatosis with polyangiitis (GPA); microscopic polyangiitis (MPA).

173. A method for treating a disease in a patient in need thereof comprising, administering to the patient, an effective amount of a compound of any one of clauses 1-105, or the composition of clause 106, wherein the disease is giant cell arteritis, polyarteritis nodosa, anti-GBM disease (Goodpasture's), systemic scleroderma, diabetic nephropathy, diabetic neuropathy, diabetic retinopathy, diabetic ulcers, Duchenne muscular dystrophy, bronchiolitis obliterans, atopic dermatitis, pyoderma gangrenosum, sweet's syndrome, dermatomyositis/polymyositis, neutrophilic dermatoses, thrombosis, bronchopulmonary dysplasia, amyotrophic lateral sclerosis, sickle cell anemia, psoriasis, or a ventilator-induced lung injury.

### Numbered Clauses—Set 2

1. A compound of formula (I)

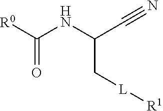

or a pharmaceutically acceptable salt or deuterated form thereof,
wherein:
R⁰ is
   5-12 membered monocyclic heterocyclyl containing 1-3 heteroatoms selected from N, S, or O, wherein the monocyclic heterocycle is optionally substituted with 1-3 R⁸, or
   5-12-membered polycyclic heterocyclyl containing 1-3 heteroatoms selected from N, S, or O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
L is polycyclic cycloalkylene, polycyclic arylene, or polycyclic heteroarylene, wherein a first atom of a first ring of the polycyclic arylene or heteroarylene is connected to

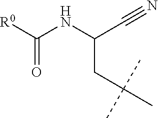

and a second atom of a second ring of the polycyclic arylene or heteroarylene is connected to R¹;
wherein L is independently substituted by 0-4 R¹⁰;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH;
R¹ is
   5-12 membered carbocyclyl optionally substituted with 1-3 R^{g},
   6-18 membered aryl optionally substituted with 1-3 R^{g},
   5-12 membered monocyclic heterocyclyl containing 1-3 heteroatoms selected from N, S or O, wherein the monocyclic heterocyclyl is optionally substituted with 1-3 R^{g},
   5-12 membered monocyclic heteroaryl containing 1-3 heteroatoms selected from N, Sor O, wherein the monocyclic heteroaryl is optionally substituted with 1-3 R^{g},
   7-14 membered bicyclic heteroaryl containing 1-3 heteroatoms selected from N, S or O, wherein the bicyclic heteroaryl is optionally substituted with 1-4 R^{g}, or
   7-20 membered tricyclic heteroaryl containing 1-3 heteroatoms selected from N, S or O, wherein the tricyclic heteroaryl is optionally substituted with 1-5 R^{g},
each R^{g} is independently selected from hydrogen, SF₅, ═O, halogen, cyano, hydroxyl, nitro, NH₂, —COOH, C₁₋₆ alkyl, C₁₋₆ alkoxy, C₃₋₆ cycloalkyloxy, C₂₋₆alkenyloxy, C₂₋₆ alkynyloxy, C₃₋₆ cycloalkyl, C₂₋₆ alkenyl, C₂₋₅ alkynyl, —NHC₁₋₆ alkyl, —N(C₁₋₆ alkyl)₂, —COC₁₋₆alkyl, —COOC₁₋₆ alkyl, —CONH₂, —CONHC₁₋₆ alkyl, —CONHC₃₋₆ cycloalkyl, —CON(C₁₋₆ alkyl)₂, —NHCOC₁₋₆alkyl, —NHCOC₃₋₅cycloalkyl, —P(O)(C₁₋₆ alkyl), —S(O)C₁₋₆alkyl, —S(O)₂C₁₋₆ alkyl, —S(O)₂C₃₋₆cycloalkyl, —SO₂-3-7 membered heterocyclyl, —S(O)NH₂, —S(O)NHC₁₋₆ alkyl, —S(O)N(C₁₋₆ alkyl)₂, —S(O)₂NH₂, —OSO₂—C₁₋₆alkyl, C₁₋₆alkylene-O—C₁₋₆alkyl, C₃₋₈-cycloalkenyloxy, aryl, heteroaryl, 3-7-membered heterocycle containing 1-3 heteroatoms selected from N, S and O, and the R^{g} is optionally further substituted with 1-3 groups selected from C₁₋₆ alkyl, C₁₋₆ haloalkyl, C₁₋₆-alkylene-OH, —CONH₂, NH₂, C₁₋₆ alkoxy, hydroxyl, —COOH, halogen, or a 5-7-membered heterocycle containing 1-3 heteroatoms selected from N, S or O, wherein the 5-7-membered heterocycle is optionally substituted with 1-2 groups selected from ═O, halogen, cyano, C₁₋₆ alkyl, and C₁₋₆ haloalkyl.

2. The compound of clause 1, or a pharmaceutically acceptable salt or deuterated form thereof, wherein R⁰ is 5-12 membered monocyclic heterocycle containing 1-3 heteroatoms selected from N, S, or O, wherein the monocyclic heterocycle is optionally substituted with 1-3 R⁸.

3. The compound of clause 1 or 2, or a pharmaceutically acceptable salt or deuterated form thereof, wherein R⁰ is

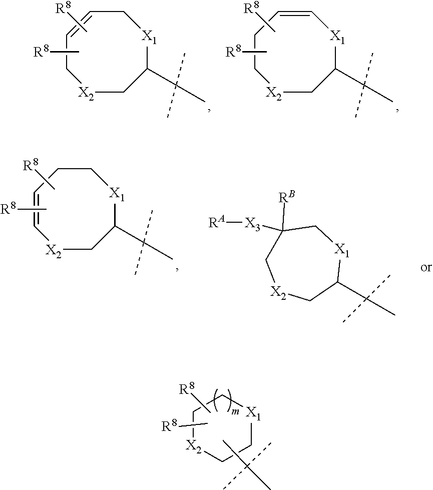

X₁ and X₂ are independently O, S, NH, N(C₁₋₆alkyl), or CR¹²R¹³, wherein at least one of X₁ and X₂ are not CR¹²R¹³;
X₃ is O, S, NH, or N(C₁₋₆alkyl);
R⁸ is H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy or halogenated C₁₋₆alkoxy;
R¹² is H, halogen, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy or halogenated C₁₋₆alkoxy
R¹³ is independently H, halogen or C₁-C₆ alkyl;
R^{A} is H, C₁₋₆alkyl, C₁₋₆alkylene-carbocyclyl, or C₁₋₆alkylene-heteroaryl; and
R^{B} is C₁₋₆alkyl, C₂₋₆alkenyl, C₁₋₆alkylene-carbocyclyl, or C₁₋₆alkylene-heteroaryl, or
R^{A} and R^{B} are taken together to form a heterocyclyl; and
m is 0, 1, 2 or 3.

4. The compound of clause 3, or a pharmaceutically acceptable salt or deuterated form thereof, wherein R⁰ is

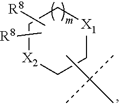

and m is 2 or 3.

5. The compound of clause 3 or 4, or a pharmaceutically acceptable salt or deuterated form thereof, wherein R⁰ is

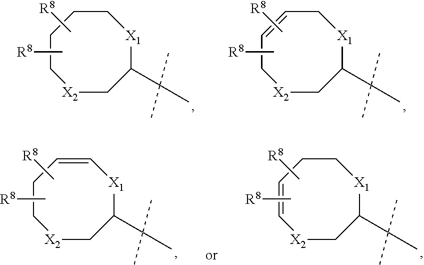

X₁ and X₂ are independently O, S, NH, N(C₁₋₆alkyl), or CR¹²R¹³, wherein at least one of X₁ and X₂ are not CR¹²R¹³;
X₃ is O, S, NH, or N(C₁₋₆alkyl);
R^{A} is H, C₁₋₆alkyl, C₁₋₆alkylene-carbocyclyl, or C₁₋₆alkylene-heteroaryl; and
R^{B} is C₁₋₆alkyl, C₂₋₆alkenyl, C₁₋₆alkylene-carbocyclyl, or C₁₋₆alkylene-heteroaryl; or
R^{A} and R^{B} are taken together to form a heterocyclyl;
R¹² is H, halogen, C₁₋₆ alkyl;
each R¹³ is independently H, halogen or C₁-C₆ alkyl.

6. The compound of clause 3, or a pharmaceutically acceptable salt or deuterated form thereof, wherein R⁰ is

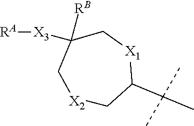

X₁ and X₂ are independently O, S, NH, N(C₁₋₆alkyl), or CR¹²R¹³, wherein at least one of X₁ and X₂ are not CR¹²R¹³,
X₃ is O, S, NH, or N(C₁₋₆alkyl),
R^{A} is H, C₁₋₆alkyl, C₁₋₆alkylene-carbocyclyl, or C₁₋₆alkylene-heteroaryl, and
R^{B} is C₁₋₆alkyl, C₂₋₆alkenyl, C₁₋₆alkylene-carbocyclyl, or C₁₋₆alkylene-heteroaryl, or
R^{A} and R^{B} are taken together to form a heterocyclyl,
R¹² is H, halogen, C₁₋₆ alkyl,
each R¹³ is independently H, halogen or C₁-C₆ alkyl.

7. The compound of clause 5, or a pharmaceutically acceptable salt or deuterated form thereof, wherein R⁰ is

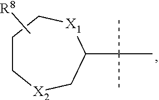

X₁ is O, X₂ is NH, and R⁸ is C₁₋₆alkoxy.

8. The compound of clause 1 or 2, wherein R⁰ is

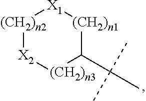

each n1, n2 and n3 is independently an integer from 0-3, and the total sum of n1, n2 and n3 is ≤4, X₁ and X₂ are independently O, S, NR⁶ or CR¹²R¹³, wherein at least one of the X₁ and X₂ is not CR¹²R¹³; each R⁶, R¹² and R¹³ is independently H, halo, or C₁-C₆ alkyl.

9. The compound of any one of clause 1-8, or a pharmaceutically acceptable salt or deuterated form thereof, wherein R⁰ is

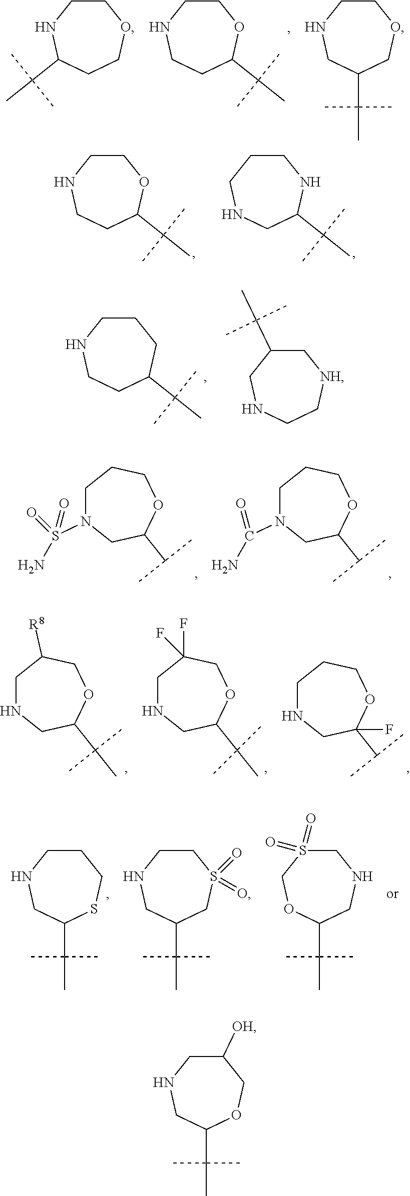

wherein R⁸ is H, F, OH, CH₃, OCH₃, OCHF₂, OCF₃, OCH₂CH₃, or —CH₂OCH₃.

10. The compound of any one of clauses 1-4, wherein R⁰ is

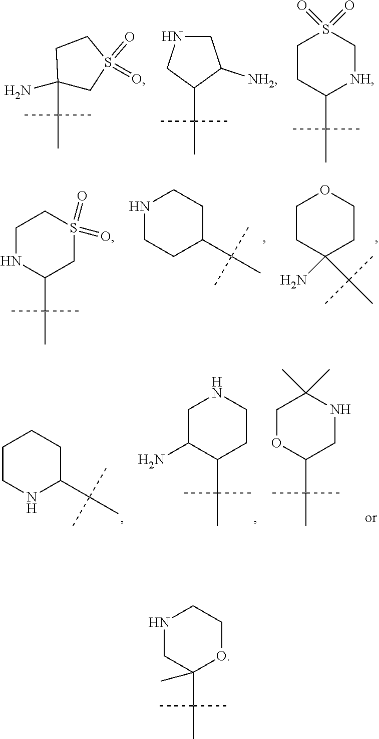

11. The compound of clause 1, or a pharmaceutically acceptable salt or deuterated form thereof, wherein R⁰ is 5-12-membered polycyclic heterocycle containing 1-3 heteroatoms selected from N, S, O, wherein the polycyclic heterocycle is optionally substituted with 1-3 R⁸.

12. The compound of clause 1 or 11, or a pharmaceutically acceptable salt or deuterated form thereof, wherein R⁰ is
5-12 membered spiro heterocycle containing 1-3 heteroatoms selected from N, S, or O, wherein the spiro heterocycle is optionally substituted with 1-3 R⁸,
5-12 membered fused heterocycle containing 1-3 heteroatoms selected from N, S, or O, wherein the spiro heterocycle is optionally substituted with 1-3 R⁸, or
7-12 membered bridged heterocycle containing 1-3 heteroatoms selected from N, S, or O, wherein the bridge heterocycle is optionally substituted with 1-3 R⁸.

13. The compound of any one of clauses 1, 11, or 12, or a pharmaceutically acceptable salt or deuterated form thereof, wherein R⁰ is 5-12 membered spiro heterocycle containing 1-3 heteroatoms selected from N, S, or O, wherein the spiro heterocycle is optionally substituted with 1-3 R⁸.

14. The compound of clause 12 or 13, or a pharmaceutically acceptable salt or deuterated form thereof, wherein R⁰ is

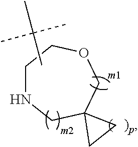

wherein
m1 and m2 are each independently 0, 1, or 2, provided that both m1 and m2 are not 0, or both m1 and m2 are not 2, and
p is 1 or 2.

15. The compound of any one of clauses 12-14, or a pharmaceutically acceptable salt or deuterated form thereof, wherein R⁰ is

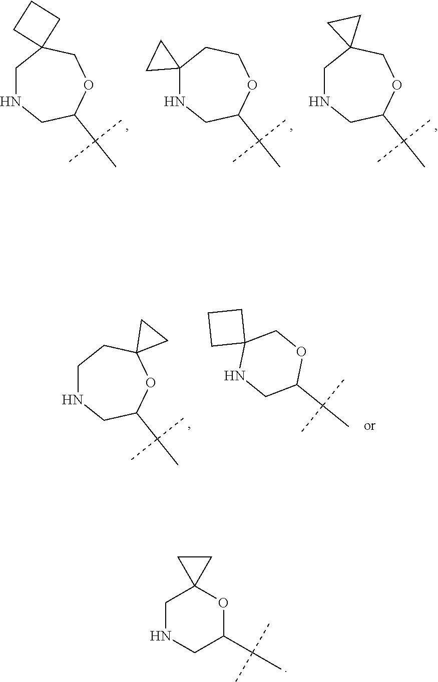

16. The compound of clause 1, 11, or 12, or a pharmaceutically acceptable salt or deuterated form thereof, wherein R⁰ is 5-12 membered fused heterocycle containing 1-3 heteroatoms selected from N, S, O, wherein the fused heterocycle is optionally substituted with 1-3 R⁸.

17. The compound of clause 16, or a pharmaceutically acceptable salt or deuterated form thereof, wherein R⁰ is

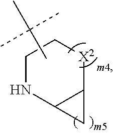

m4 is 0 or 1,
m5 is 1 or 2,
X² is O, S, NH, N(C₁₋₆alkyl), or CR¹²R¹³,
R¹² is selected from H, halogen, cyano, hydroxyl, NH₂, NH(C₁₋₆alkyl), N(C₁₋₆alkyl)₂, COOH, C₁₋₆ alkyl, —CONH₂, —S(═O)NH₂, —S(O)₂NH₂, C₁₋₆alkoxy or halogenated C₁₋₆alkoxy;
R¹³ is independently H, F, Cl, Br, I or C₁-C₆ alkyl.

18. The compound of clause 16, or a pharmaceutically acceptable salt or deuterated form thereof, wherein R⁰ is

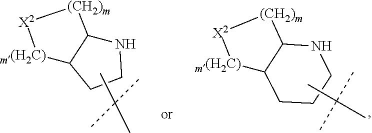

X₂ is O, S, NH, N(C₁₋₆alkyl), or CR¹²R¹³
R¹² is H, halogen, cyano, hydroxyl, NH₂, NH(C₁₋₆alkyl), N(C₁₋₆alkyl)₂, COOH, C₁₋₆ alkyl, —CONH₂, —S(═O)NH₂, —S(O)₂NH₂, C₁₋₆alkoxy or halogenated C₁₋₆alkoxy;
R¹³ is H, halogen or C₁-C₆ alkyl;
each m and m′ is independently an integer from 0-3, and the total sum of m and m′ is ≤3.

19. The compound of clause 17, or a pharmaceutically acceptable salt or deuterated form thereof, wherein R⁰ is

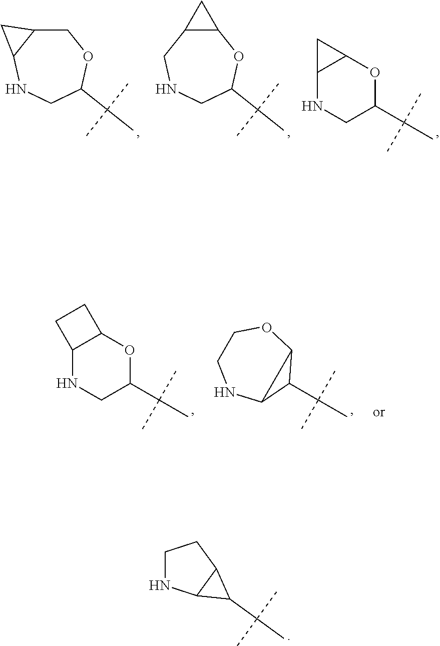

20. The compound of clause 12 or 16, or a pharmaceutically acceptable salt or deuterated form thereof, wherein R⁰ is

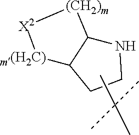

X₂ is O, S, NH, N(C₁₋₆alkyl), or CR¹²R¹³,
R¹² is H, halogen, C₁₋₆ alkyl,
R¹³ is H, halogen or C₁-C₆ alkyl, and
each m and m′ is independently an integer from 0-3, and the total sum of me and mc′ is ≤3.

21. The compound of clause 20, or a pharmaceutically acceptable salt or deuterated form thereof, wherein R⁰ is, X² is

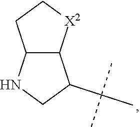

22. The compound of clause 21, or a pharmaceutically acceptable salt or deuterated form thereof, wherein R⁰ is

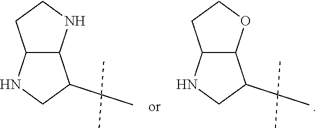

23. The compound of clause 20, or a pharmaceutically acceptable salt or deuterated form thereof, the wherein R⁰ is

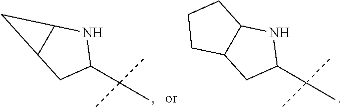

24. The compound of clause 18, or a pharmaceutically acceptable salt or deuterated form thereof, the wherein R⁰ is

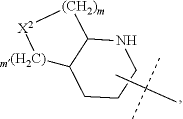

X₂ is O, S, NH, N(C₁₋₆alkyl), or CR¹²R¹³
R¹² is H, halogen, C₁₋₆ alkyl; and
R¹³ is H, halogen or C₁-C₆ alkyl.

25. The compound of clause 24, or a pharmaceutically acceptable salt or deuterated form thereof, wherein R⁰ is

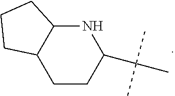

26. The compound of clause 11 or 12, or a pharmaceutically acceptable salt or deuterated form thereof, wherein R⁰ is 7-12 membered bridged heterocycle containing 1-3 heteroatoms selected from N, S, O, wherein the bridged heterocycle is optionally substituted with 1-3 R⁸.

27. The compound of clause 26, or a pharmaceutically acceptable salt or deuterated form thereof, wherein R⁰ is

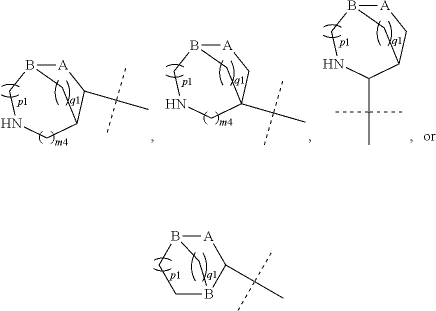

each of which is optionally substituted with 1-4 R⁸, wherein
A is a bond, —O—, —O—CH₂—, —CH₂—O—CH₂—, —CH₂OCH₂CH₂—, —CH₂—, —CH₂CH₂—, or —CH₂NH—,
B is N or CH,
m⁴ is 0 or 1,
p¹ is 0, 1, or 2,
q¹ is 1, 2, or 3.

28. The compound of clause 26 or 27, or a pharmaceutically acceptable salt or deuterated form thereof, wherein R⁰ is

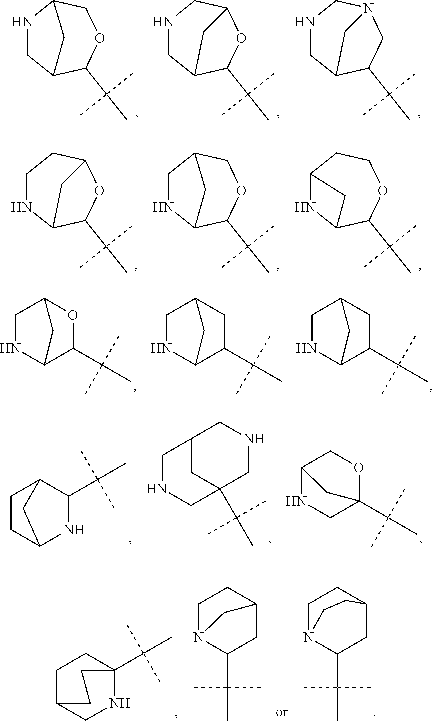

29. The compound of clause 1, or a pharmaceutically acceptable salt or deuterated form thereof, wherein R⁰ is

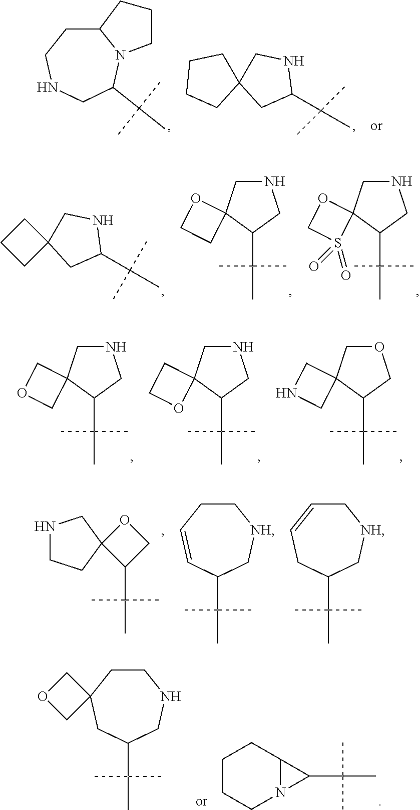

30. The compound of clause 1, or a pharmaceutically acceptable salt or deuterated form thereof, wherein R¹ is 5-12 membered carbocyclyl optionally substituted with 1-3 R^{g} group.

31. The compound of clause 30, or a pharmaceutically acceptable salt or deuterated form thereof, wherein R¹ is

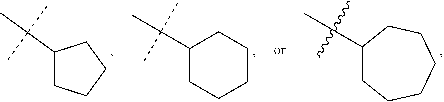

each of which is optionally substituted with 1-3 R^{g}.

32. The compound of clause 1, or a pharmaceutically acceptable salt or deuterated form thereof, wherein R¹ is 6-18 membered aryl optionally substituted with 1-3 R^{g}.

33. The compound of clause 32, or a pharmaceutically acceptable salt or deuterated form thereof, wherein R¹ is wherein L is

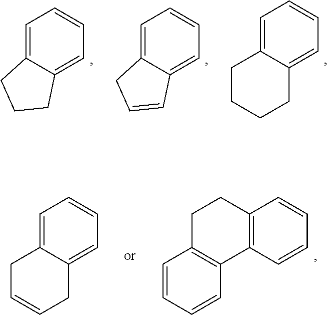

attached to R¹ by replacing any hydrogen atom of R¹, and wherein each R¹ is optionally substituted with 1-3 R^{g}.

34. The compound of clause 32, or a pharmaceutically acceptable salt or deuterated form thereof, wherein R¹ is

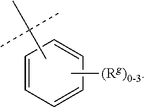

35. The compound of clause 34, or a pharmaceutically acceptable salt or deuterated form thereof, wherein R¹ is

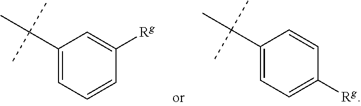

36. The compound of clause 34 or 35, or a pharmaceutically acceptable salt or deuterated thereof, wherein R¹ is

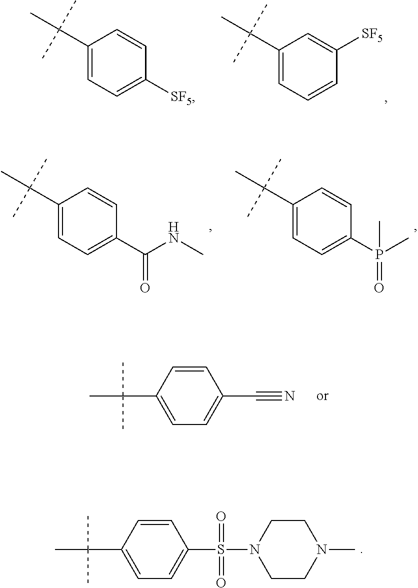

37. The compound of clause 34, or a pharmaceutically acceptable salt or deuterated form thereof, wherein R¹ is

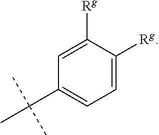

38. The compound of clause 37, or a pharmaceutically acceptable salt or deuterated form thereof, wherein R¹ is

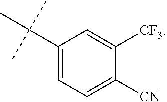

39. The compound of clause 1, or a pharmaceutically acceptable salt or deuterated form thereof, wherein R¹ is 5-12 membered monocyclic heterocyclyl containing heteroatoms selected from N, S or O, wherein the monocyclic heterocyclyl is optionally substituted with 1-3 R^{g} group.

40. The compound of clause 39, or a pharmaceutically acceptable salt or deuterated form thereof, wherein R¹ is

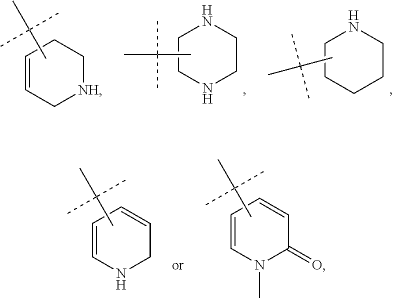

wherein each one of R¹ is optionally substituted with 1-3 R^{g}.

41. The compound of clause 1, or a pharmaceutically acceptable salt or deuterated form thereof, wherein R¹ is 5-12 membered monocyclic heteroaryl containing heteroatoms selected from N, S or O, wherein each one of the monocyclic heteroaryl is optionally substituted with 1-3 R^{g} group.

42. The compound of clause 41, or a pharmaceutically acceptable salt or deuterated form thereof, wherein R¹ is

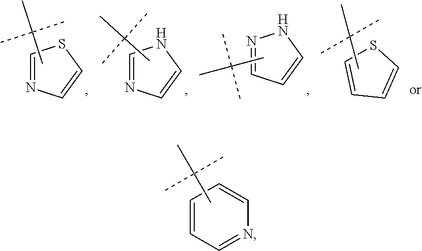

each of which is optionally substituted with 1-3 R^{g} group.

43. The compound of clause 41 or 42, or a pharmaceutically acceptable salt or deuterated form thereof, wherein each R^{g} is independently H, halogen, C₁-C₆ alkyl, OSO₂C₁₋₆alkyl, or CN.

44. The compound of clause 42 or 43, or a pharmaceutically acceptable salt or deuterated form thereof, wherein R¹ is

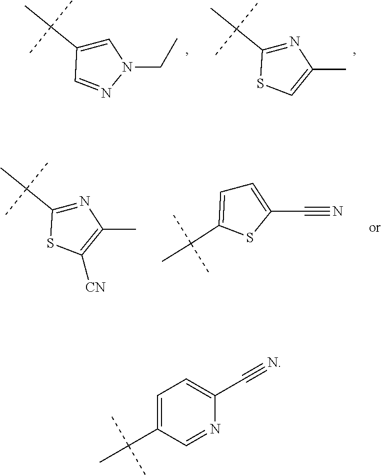

45. The compound of clause 1, or a pharmaceutically acceptable salt or deuterated form thereof, wherein R^{g} is R⁶ or R⁷, wherein each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH.

47. The compound of clause 45, or a pharmaceutically acceptable salt or deuterated form thereof, wherein R¹ is

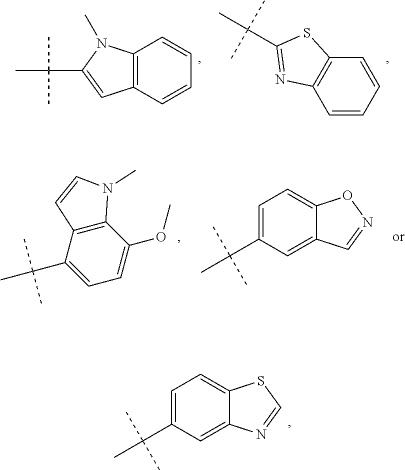

each of which is optionally substituted with 1-3 R^{g} group.

48. The compound of clause 45 or 46, or a pharmaceutically acceptable salt or deuterated form thereof, wherein R¹ is

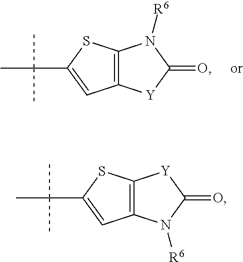

wherein
Y is independently O, S, CHR⁶ or NR⁶; and
R⁶ is C₁₋₆alkyl optionally substituted by 1, 2 or 3 F, or by OH, O—C₁₋₆alkyl, N(C₁₋₆alkyl)₂, cycloalkyl, or heterocyclyl.

49. The compound of clause 48, or a pharmaceutically acceptable salt or deuterated form thereof, wherein R¹ is

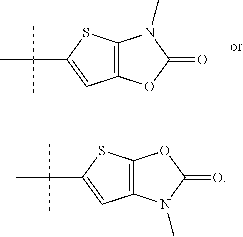

50. The compound of clause 45 or 46, or a pharmaceutically acceptable salt or deuterated form thereof, wherein R¹ is

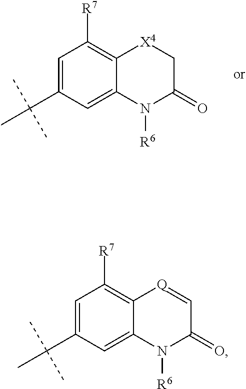

X⁴ is NR⁶, O, CR⁷, CR¹⁴R¹⁵, S, S(O) or S(O)₂,
Q is CH or N,
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH;
R¹⁴ and R¹¹ are each independently selected from H, halogen, C₁₋₆ alkyl, C₁₋₆alkoxy, C₂₋₆ alkenyl, C₂₋₆ alkynyl, C₂₋₆alkenyloxy, C₂₋₆ alkynyloxy, C₃₋₆ cycloalkyl, C₃₋₆ cycloalkoxy, cyano, hydroxyl, NH₂, COOH, —S(O)C₁₋₆alkyl, —S(O)₂C₁₋₆ alkyl, —S(O)₂C₃₋₆cycloalkyl, —SO₂-3-7 membered heterocyclyl, and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups selected from halogen, cyano, hydroxyl, NH₂ and —COOH;
alternatively, R¹⁴ and R¹⁵ form ═O.

51. The compound of any one of clauses 45-46, or a pharmaceutically acceptable salt or deuterated form thereof, wherein R¹ is

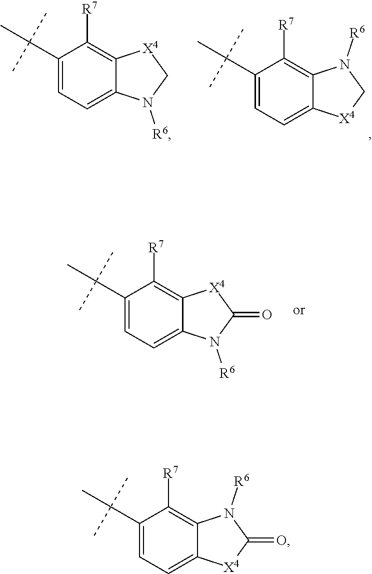

each X⁴ is independently NR⁶, O, CR¹⁴R¹⁵, S, S(O) or S(O)₂,
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH;
R¹⁴ and R¹¹ are each independently selected from H, halogen, C₁₋₆ alkyl, C₁₋₆alkoxy, C₂₋₆ alkenyl, C₂₋₆ alkynyl, C₂₋₆alkenyloxy, C₂₋₆ alkynyloxy, C₃₋₆ cycloalkyl, C₃₋₆ cycloalkoxy, cyano, hydroxyl, NH₂, COOH, —S(O)C₁₋₆alkyl, —S(O)₂C₁₋₆ alkyl, —S(O)₂C₃₋₆cycloalkyl, —SO₂-3-7 membered heterocyclyl, and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups selected from halogen, cyano, hydroxyl, NH₂ and —COOH;
alternatively, R¹⁴ and R¹⁵ form ═O.

52. The compound of clause 50, or a pharmaceutically acceptable salt or deuterated form thereof, wherein
each X⁴ is independently O, S, NR⁶ or CR¹⁴R⁵;
R⁶ is C₁₋₆alkyl optionally substituted by 1, 2 or 3 F, or by OH, O—C₁₋₆alkyl, N(C₁₋₆alkyl)₂, cycloalkyl, or heterocyclyl;
R⁷ is H, halo or C₁₋₆alkyl,
R¹⁴ and R¹⁵ are independently H, halo or C₁₋₆alkyl.

53. The compound of clause 52 or 53, or a pharmaceutically acceptable salt or deuterated form thereof, wherein each X⁴ is independently NH, O, S, CHF, or CHF₂.

54. The compound of any one of clauses 52-53, or a pharmaceutically acceptable salt or deuterated form thereof, wherein R¹ is

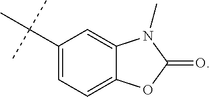

55. The compound of any one of clauses 52-53, or a pharmaceutically acceptable salt or deuterated form thereof, wherein R¹ is

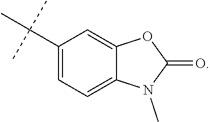

56. The compound of any one of clauses 45-56, or a pharmaceutically acceptable salt or deuterated form thereof, wherein R¹ is

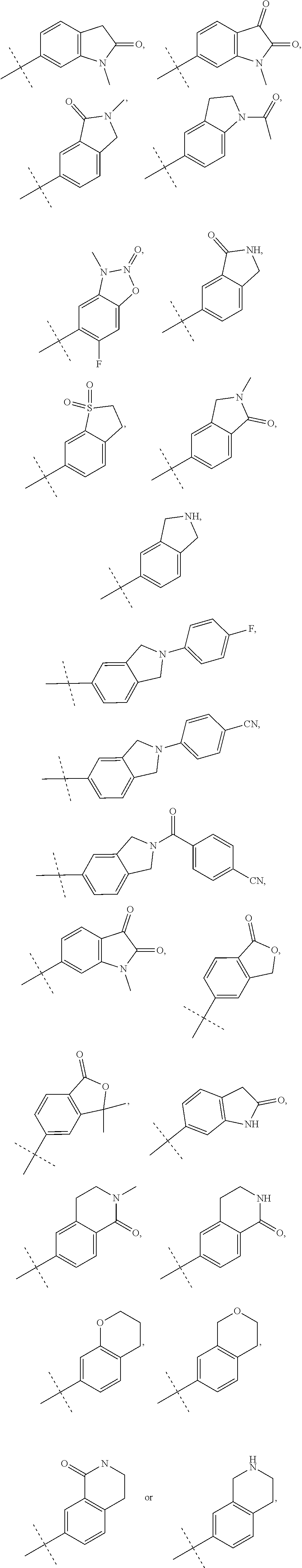

57. The compound of clause 45 or 46, or a pharmaceutically acceptable salt or deuterated form thereof, wherein R¹ is

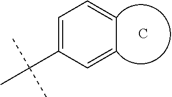

optionally substituted with 1-4 R⁶ or R⁷ group, wherein ring C is a 7-8 membered heterocycle comprising 1-3 heteroatoms selected from O, S and N.

58. The compound of clause 57, or a pharmaceutically acceptable salt or deuterated form thereof, wherein R¹ is

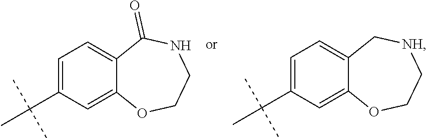

each of which is optionally substituted with 1-4 R⁶ or R⁷.

59. The compound of clause 57 or 58, or a pharmaceutically acceptable salt or deuterated form thereof, wherein R¹ is

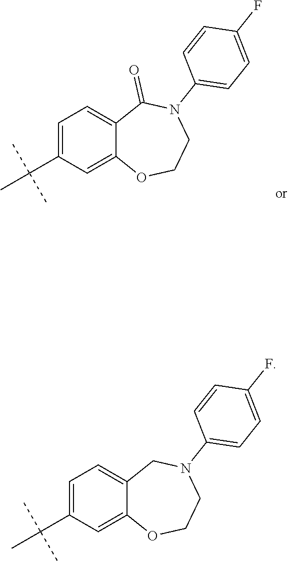

60. The compound of clause 1, or a pharmaceutically acceptable salt or deuterated form thereof, wherein R¹ is 7-20 membered tricyclic heteroaryl containing heteroatoms selected from N, S or O, wherein the tricyclic heteroaryl is optionally substituted with 1-5 R^{g}, wherein R^{g} is R⁶, R⁷, R¹¹, R¹⁴ or R¹⁵.

61. The compound of clause 1 or 60, or a pharmaceutically acceptable salt or deuterated form thereof, wherein R¹ is 7-20 membered spiro tricyclic heteroaryl containing heteroatoms selected from N, S or O, wherein the tricyclic heteroaryl is optionally substituted with 1-5 R^{g}.

62. The compound of any one of clauses 60-61, or a pharmaceutically acceptable salt or deuterated form thereof, wherein R¹ is

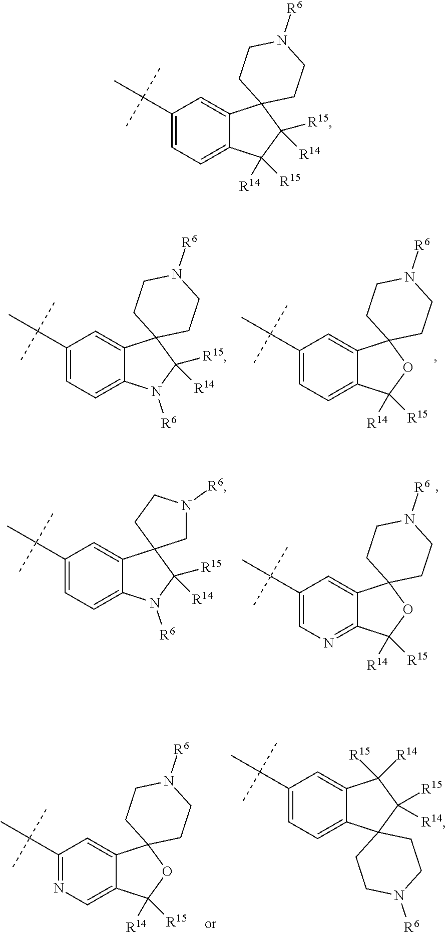

wherein
each R⁶ is independently selected from H, C₁₋₆ alkyl, —COC₁₋₆ alkyl, C₂₋₆ alkenyl, C₂₋₆alkynyl, C₃₋₆cycloalkyl or alkylene-O-alkyl; wherein the alkyl, alkenyl, alkynyl and cycloalkyl is optionally substituted with 1-3 groups selected from halogen, cyano, hydroxyl, NH₂ and COOH; and,
R¹⁴ and R¹¹ are each independently selected from H, halogen, C₁₋₆ alkyl, C₁₋₆alkoxy, C₂₋₆ alkenyl, C₂₋₆ alkynyl, C₂₋₆alkenyloxy, C₂₋₆ alkynyloxy, C₃₋₆ cycloalkyl, C₃₋₆ cycloalkoxy, cyano, hydroxyl, NH₂, COOH, —S(O)C₁₋₆alkyl, —S(O)₂C₁₋₆ alkyl, —S(O)₂C₃₋₆cycloalkyl, —SO₂-3-7 membered heterocyclyl and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups selected from halogen, cyano, hydroxyl, NH₂ and —COOH;
alternatively, R¹⁴ and R¹⁵ form ═O.

63. The compound of clause 61-62, or a pharmaceutically acceptable salt or deuterated form thereof, wherein R¹ is

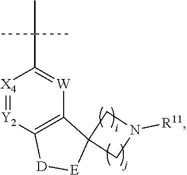

wherein
W, X₄ and Y₂ are each independently CH or N, provided that a maximum of one of W, X₄ and Y₂ can be N;
D-E is N(H)—C(═O), N(C₁₋₆alkyl)-C(═O), CH₂CH₂, C(═O)-0 or CH₂—O;
R¹¹ is H, C₁₋₆alkyl, alkylene-O-alkyl, or heterocyclyl; and
i and j are each independently 1, 2 or 3; provided that the sum of i+j is 2, 3 or 4.

64. The compound of any one of clauses 60-63, or a pharmaceutically acceptable salt or deuterated form thereof, wherein R¹ is

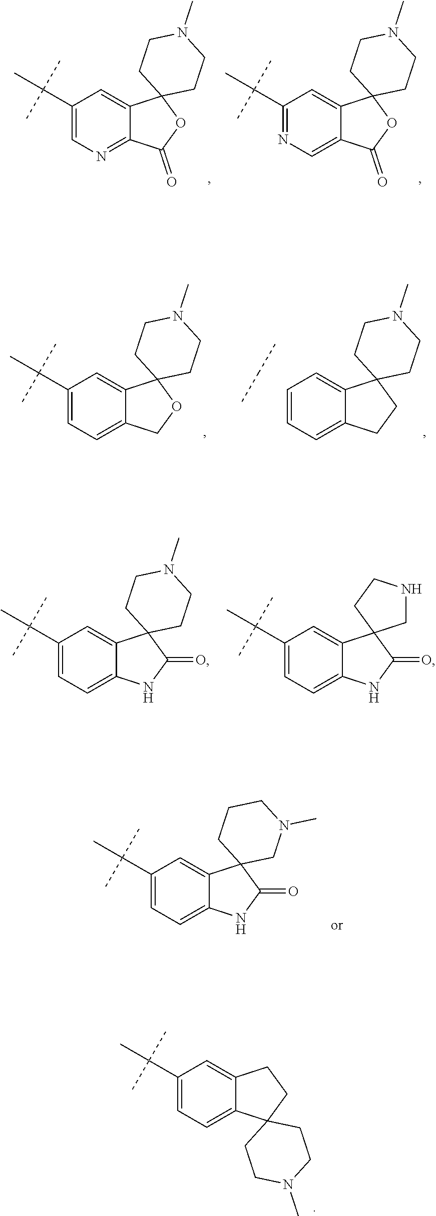

65. The compound of clause 1, or a pharmaceutically acceptable salt or deuterated form thereof, wherein R¹ is 7-20 membered fused tricyclic heteroaryl containing heteroatoms selected from N, S or O, wherein the tricyclic heteroaryl is optionally substituted with 1-5 R^{g}.

66. The compound of clause 1 or 65, or a pharmaceutically acceptable salt or deuterated form thereof, wherein R¹ is

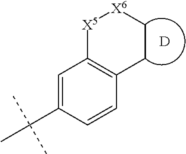

optionally substituted with 1-5 R^{g}, wherein
X⁵ and X⁶ are each independently selected from single bond, —C(R¹⁴R¹⁵)—O—, —C(R¹⁴R¹⁵)—C(R¹⁴R¹⁵)—, —OC(R¹⁴R¹⁵)—, —C(R¹⁴R¹⁵)—, —O—, and —NR⁶—;
R¹⁴ and R¹⁵ are each independently selected from H, halogen, C₁₋₆ alkyl, C₁₋₆alkoxy, C₂₋₆ alkenyl, C₂₋₆ alkynyl, C₂₋₆alkenyloxy, C₂₋₆ alkynyloxy, C₃₋₆ cycloalkyl, C₃₋₆ cycloalkoxy, cyano, hydroxyl, NH₂, COOH, —S(O)C₁₋₆alkyl, —S(O)₂C₁₋₆ alkyl, —S(O)₂C₃₋₆cycloalkyl, —SO₂-3-7 membered heterocyclyl and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups selected from halogen, cyano, hydroxyl, NH₂ and —COOH;
each R⁶ is independently selected from H, C₁₋₆ alkyl, —COC₁₋₆ alkyl, C₂₋₆ alkenyl, C₂₋₆alkynyl, C₃₋₆cycloalkyl or alkylene-O-alkyl; wherein the alkyl, alkenyl, alkynyl and cycloalkyl is optionally substituted with 1-3 groups selected from halogen, cyano, hydroxyl, NH₂ and COOH;
Ring D is selected from aryl and 5-8 membered heteroaryl containing 1-3 heteroatoms, wherein aryl and said 5-8 membered heteroaryl containing 1-3 heteroatoms each independently optionally substituted with 1-3 R^{g},
provided when ring D is aryl, X⁵ and X⁶ are not both —C(R¹⁴R¹⁵)—C(R¹⁴R¹⁵)— or —C(R¹⁴R¹⁵)—.

67. The compound of clause 66, or a pharmaceutically acceptable salt or deuterated form thereof, wherein R¹ is

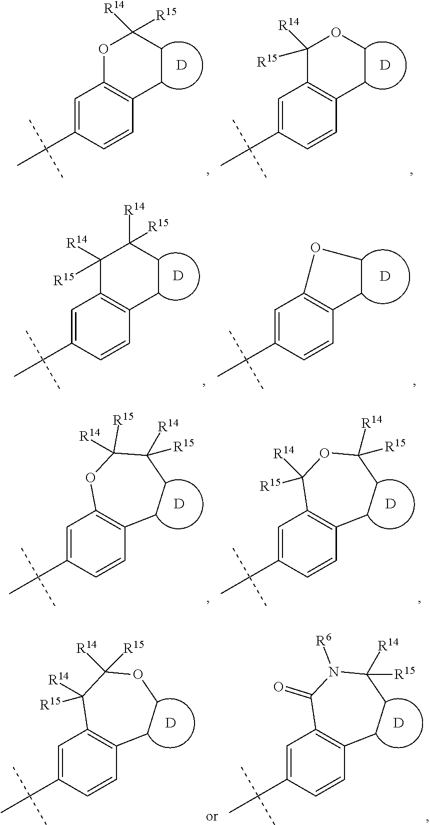

each of which is optionally substituted with 1-5 R^{g}.

68. The compound of clause 66 or 67, or a pharmaceutically acceptable salt or deuterated form thereof, wherein Ring D is

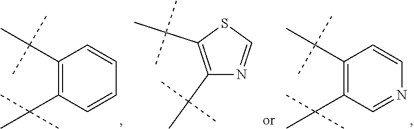

each of which is optionally substituted with 1-3 R^{g} group.

69. The compound of any one of clauses 66-68, or a pharmaceutically acceptable salt deuterated form thereof, wherein R¹ is

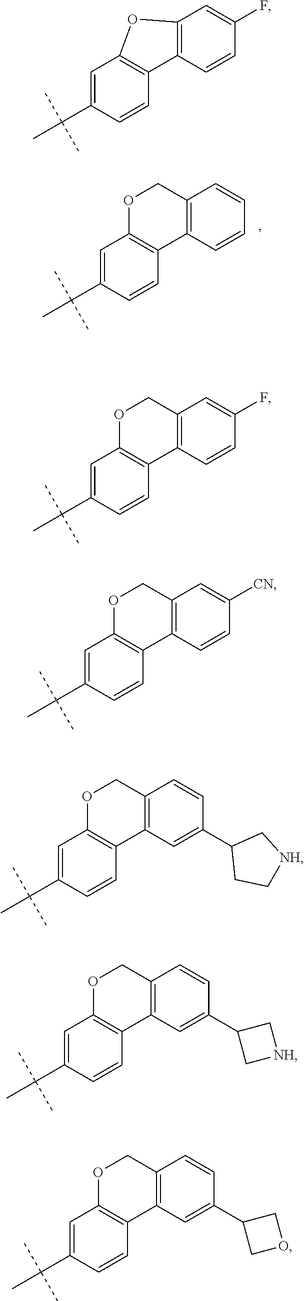

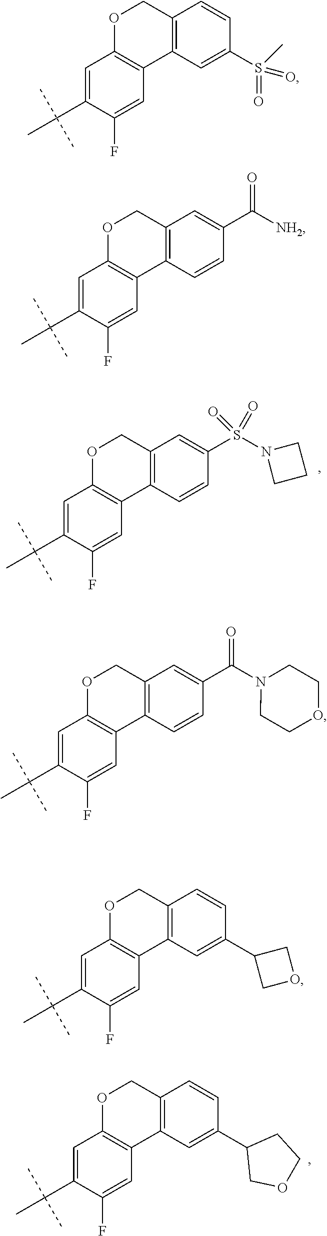

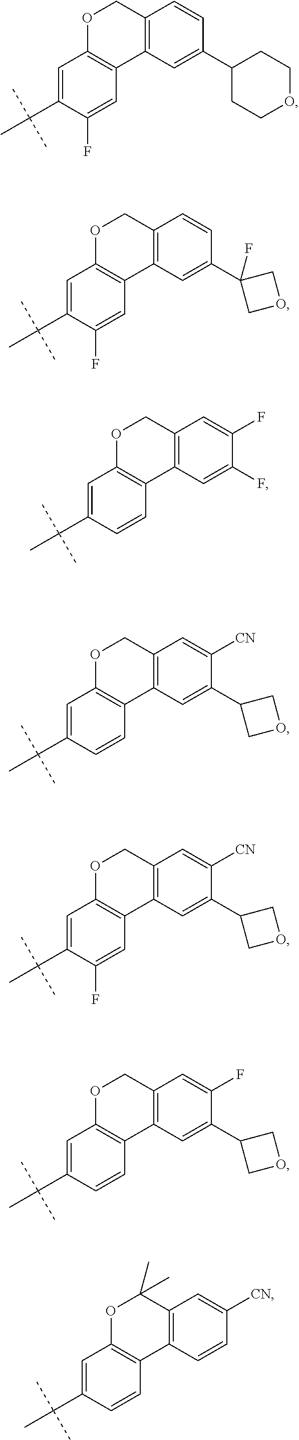

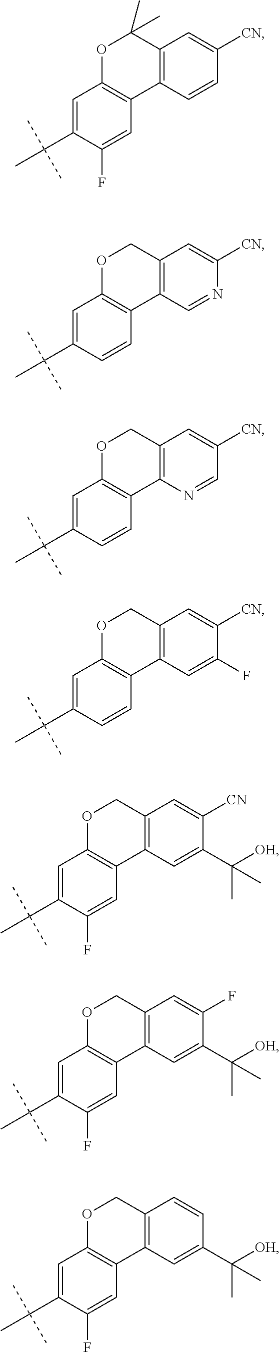

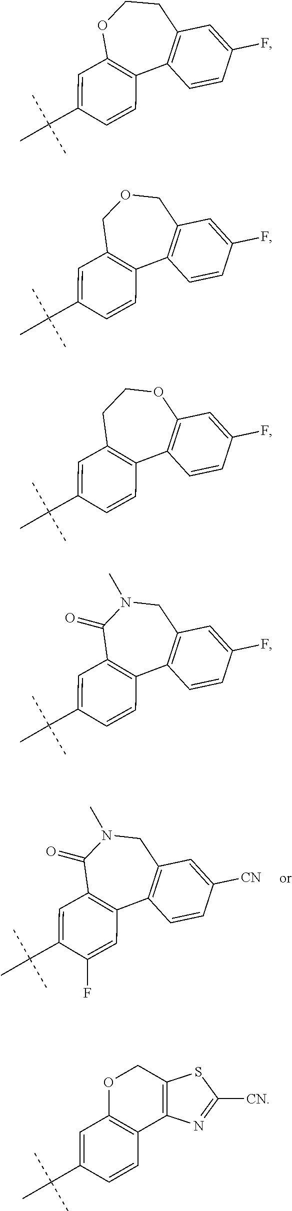

70. The compound of any one of clauses 1-69, or a pharmaceutically acceptable salt or deuterated form thereof, wherein L is a polycyclic cycloalkylene.

71. The compound of any one of clauses 1-70, or a pharmaceutically acceptable salt or deuterated form thereof, wherein the polycyclic cycloalkylene comprises 4-10 carbon atoms.

72. The compound of any one of clauses 1-71, or a pharmaceutically acceptable salt or deuterated form thereof, wherein the polycyclic cycloalkylene is connected to R¹ at a first quaternary carbon of the polycyclic cycloalkylene and independently connected to

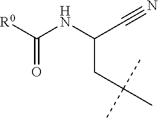

through a second quaternary carbon of the polycyclic cycloalkylene.

73. The compound of any one of clauses 1-72, or a pharmaceutically acceptable salt or deuterated form thereof, wherein the polycyclic cycloalkylene is

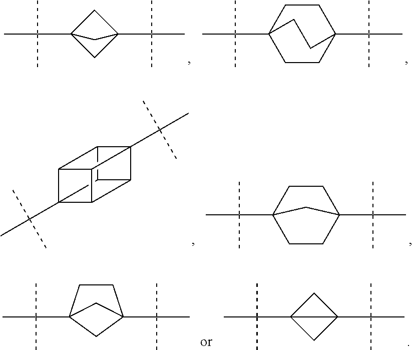

74. The compound of any one of clauses 1-73, or a pharmaceutically acceptable salt or deuterated form thereof, wherein the polycyclic cycloalkylene is

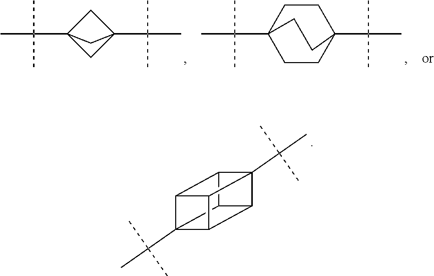

75. The compound of any one of clauses 1-74, or a pharmaceutically acceptable salt or deuterated form thereof, wherein L is a polycyclic heteroarylene substituted by 0-4 R¹⁰.

76. The compound of clause 75, or a pharmaceutically acceptable salt or deuterated form thereof, wherein the polycyclic heteroarylene comprises 8-12 ring atoms and is substituted by 0-4 R¹⁰.

77. The compound of any one of clauses 75-76, or a pharmaceutically acceptable salt or deuterated form thereof, wherein the polycyclic heteroarylene comprises 8-10 ring atoms and is substituted by 0-4 R¹⁰.

78. The compound of any one of clauses 75-77, or a pharmaceutically acceptable salt or deuterated form thereof, wherein the polycyclic heteroarylene comprises 8-9 ring atoms and is substituted by 0-4 R¹⁰.

79. The compound of clause 75, or a pharmaceutically acceptable salt or deuterated form thereof, wherein the polycyclic heteroarylene is

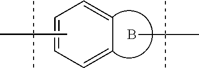

substituted by 0-4 R¹⁰, wherein ring B is a heteroaryl ring.

80. The compound of any one of clauses 75-79, or a pharmaceutically acceptable salt or deuterated form thereof, wherein the polycyclic heteroarylene comprises 9 ring atoms and 1 or 2 heteroatoms selected from O, N, or S, and the polycyclic heteroarylene is substituted by 0-4 R¹⁰.

81. The compound of any one of clauses 75-80, or a pharmaceutically acceptable salt or deuterated form thereof, wherein the polycyclic heteroarylene is

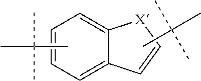

substituted by 0-4 R¹⁰, wherein X′ is O, S, NH or N(C₁₋₆ alkyl).

82. The compound of any one of clauses 75-81, or a pharmaceutically acceptable salt or deuterated form thereof, wherein the polycyclic heteroarylene is benzothienylene, indolylene or benzofuranylene substituted by 0-4 R¹⁰.

83. The compound of any one of clauses 75-82, or a pharmaceutically acceptable salt or deuterated form thereof, wherein the polycyclic heteroarylene is

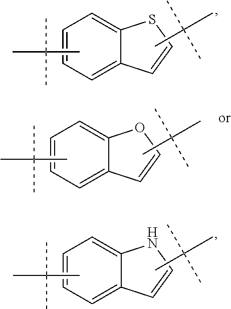

each of which is substituted by 0-4 R¹⁰.

84. The compound of clause 83, or a pharmaceutically acceptable salt or deuterated form thereof, wherein

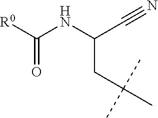

is attached to the phenyl ring of

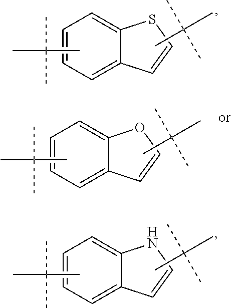

and R¹ is attached the 5-membered ring of

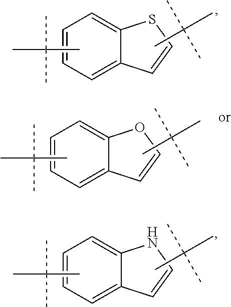

wherein

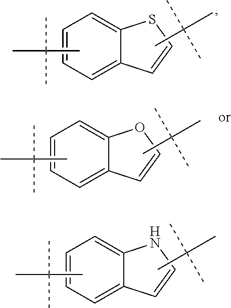

or is substituted by 0-4 R¹⁰.

85. The compound of clause 83, or a pharmaceutically acceptable salt or deuterated form thereof, wherein

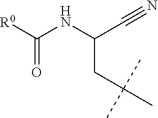

is attached the 5-membered ring of

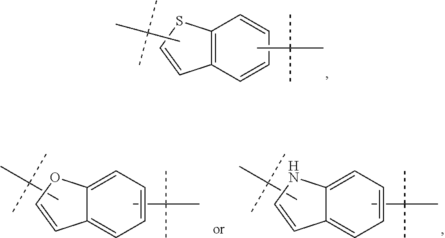

and R¹ is attached to the phenyl ring of

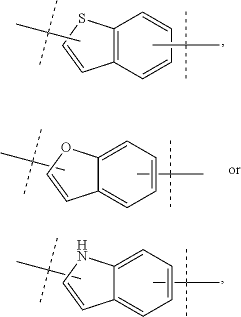

and wherein

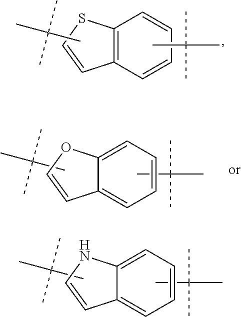

is substituted by 0-4 R¹⁰.

86. The compound of any one of clauses 75-85, or a pharmaceutically acceptable salt or deuterated form thereof, wherein the polycyclic heteroarylene is

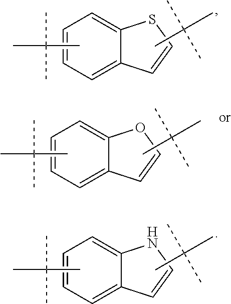

87. The compound of any one of clauses 75-86, or a pharmaceutically acceptable salt or deuterated form thereof, wherein the polycyclic heteroarylene is

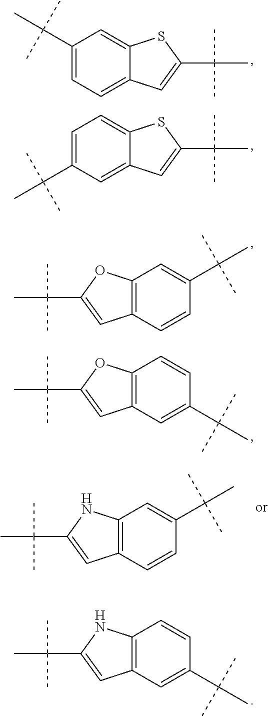

88. The compound of any one of clauses 75-86, or a pharmaceutically acceptable salt or deuterated form thereof, wherein L is

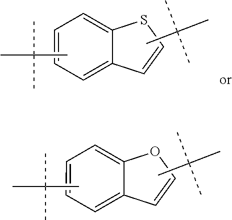

substituted by 1-4 R¹⁰.

89. The compound of any one of clauses 88, or a pharmaceutically acceptable salt or deuterated form thereof, wherein L is

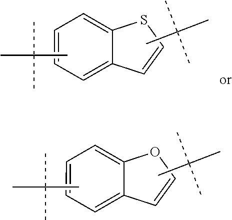

or substituted by one R¹⁰.

90. The compound of any one of clauses 88 or 89, or a pharmaceutically acceptable salt or deuterated form thereof, wherein R¹⁰ is halo.

91. The compound of any one of clauses 88-90, or a pharmaceutically acceptable salt or deuterated form thereof, wherein L is

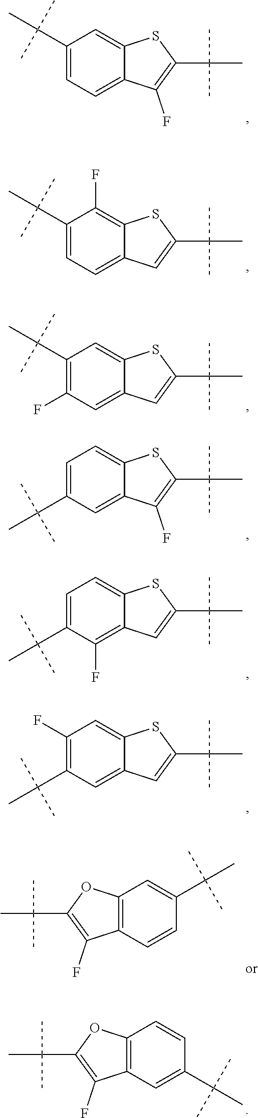

92. The compound of any one of clauses 75-78, or a pharmaceutically acceptable salt or deuterated form thereof, wherein the polycyclic heteroarylene comprises 8 ring atoms and 1 or 2 heteroatoms selected from O, N, or S, and the polycyclic heteroarylene is substituted by 0-4 R¹⁰.

93. The compound of any one of clauses 75-88 or 92, or a pharmaceutically acceptable salt or deuterated form thereof, wherein the polycyclic heteroarylene is

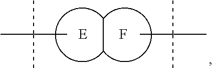

wherein each of ring E and ring F is 5-membered heteroaryl comprising 1-3 heteroatoms selected from O, N or S.

94. The compound of any one of clauses 75-88 or 92-93, or a pharmaceutically acceptable salt or deuterated form thereof, wherein the polycyclic heteroarylene is

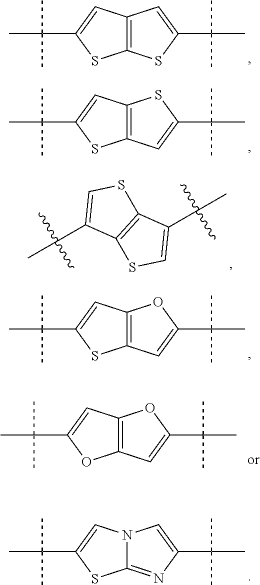

95. The compound of any one of clause 1-69, or a pharmaceutically acceptable salt or deuterated form thereof, wherein L is polycyclic arylene.

96. The compound of clause 95, or a pharmaceutically acceptable salt or deuterated form thereof, wherein L is

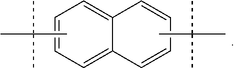

97. The compound of clause 95 or 96, or a pharmaceutically acceptable salt or deuterated form thereof, wherein L is

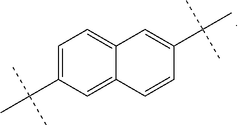

98. The compound of any one of clauses 1-69, wherein the compound is of Formula (II),

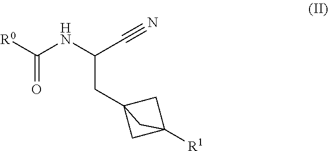

or a pharmaceutically acceptable salt or deuterated form thereof.

99. The compound of clause 1 wherein the compound is of Formula (III),

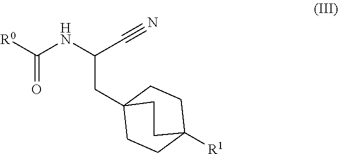

or a pharmaceutically acceptable salt or deuterated form thereof.

100. The compound of clause 1, wherein the compound is of Formula (IV),

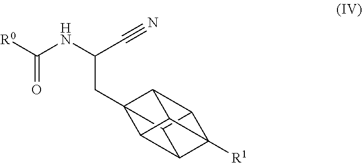

or a pharmaceutically acceptable salt or deuterated form thereof.

101. The compound of clause 1, wherein the compound is of Formula (V),

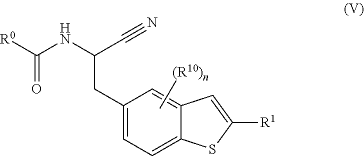

or a pharmaceutically acceptable salt or deuterated form thereof,
wherein n is 0 or 1, and R¹⁰ is substituted on the phenyl ring or the thiophenyl ring of the benzothienylene ring.

102. The compound of clause 1, wherein the compound is of Formula (VI),

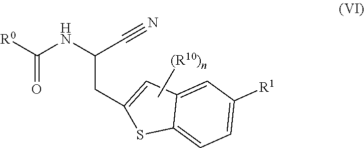

or a pharmaceutically acceptable salt or deuterated form thereof,
wherein n is 0 or 1, and R¹⁰ is substituted on the phenyl ring or the thiophenyl ring of the benzothienylene ring.

103. The compound of clause 1, wherein the compound is of Formula (VII),

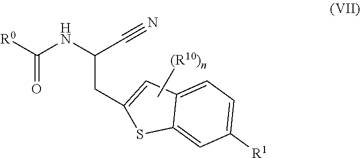

or a pharmaceutically acceptable salt or deuterated form thereof,
wherein n is 0 or 1, and R¹⁰ can be substituted on the phenyl ring or the thiophenyl ring of the benzothienylene ring.

104. The compound of anyone clauses 98-103, or a pharmaceutically acceptable salt or deuterated form thereof, wherein n is 0.

105. The compound of any one of clauses 98-103, or a pharmaceutically acceptable salt or deuterated form thereof, wherein n is 1 and R¹⁰ is halo.

106. The compound of any one of clauses 98-103 or 105, or a pharmaceutically acceptable salt or deuterated form thereof, wherein n is 1 and R¹⁰ is F.

107. The compound of any one of clauses 1-106, or a pharmaceutically acceptable salt or deuterated form thereof, wherein
R¹ is

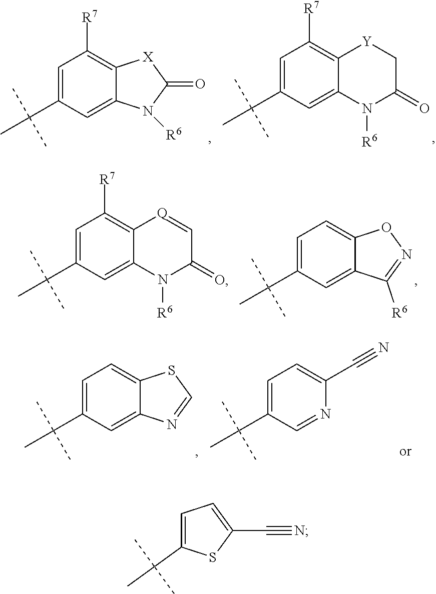

X is O, S or CF₂;
Y is O or S;
Q is CH or N;
R⁶ is C₁₋₃alkyl, wherein said C₁₋₃alkyl is optionally substituted by 1, 2 or 3 F, OH, OC₁₋₃alkyl, N(C₁₋₃alkyl)₂, cyclopropyl, or tetrahydropyran; and
R⁷ is H, F, C₁ or CH₃.

108. The compound of any one of clauses 1-107, or a pharmaceutically acceptable salt or deuterated form thereof, wherein
R¹ is

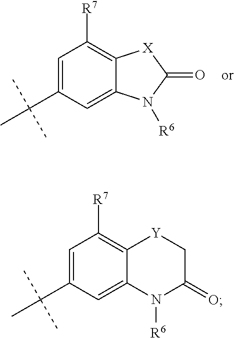

X is O, S or CF₂;
Y is O or S;
R⁶ is C₁₋₃alkyl, wherein said C₁₋₃alkyl is optionally substituted by 1, 2 or 3 F, OH, OC₁₋₃alkyl, N(C₁₋₃alkyl)₂, cyclopropyl, or tetrahydropyran; and
R⁷ is H, F, C₁ or CH₃.

109. The compound of clause 1-107, or a pharmaceutically acceptable salt or deuterated form thereof, wherein R¹ is

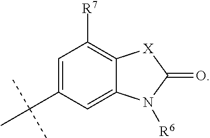

110. The compound of clause 109, or a pharmaceutically acceptable salt or deuterated form thereof, wherein, X is O; R⁶ is C₁₋₃alkyl; and R⁷ is H.

111. The compound of any one of clauses 1-107, or a pharmaceutically acceptable salt or deuterated form thereof, wherein
R¹ is

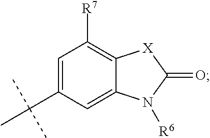

X is O;
R⁶ is C₁₋₃alkyl, wherein said C₁₋₃alkyl is optionally substituted by 1, 2 or 3 F; and
R⁷ is H.

112. The compound of any one of clauses 1-107, or a pharmaceutically acceptable salt or deuterated form thereof, wherein,
R¹ is

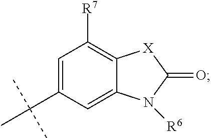

X is O;
R⁶ is C₁₋₃alkyl; and
R⁷ is H.

113. The compound of any one of clauses 1-107, or a pharmaceutically acceptable salt thereof, wherein R¹ is

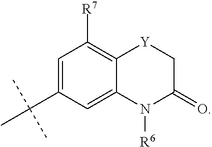

114. The compound of any one of clauses 1-107, or a pharmaceutically acceptable salt or deuterated form thereof, wherein R¹ is

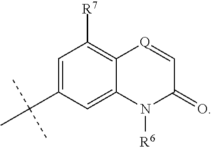

115. The compound of any one of clauses 1-107, or a pharmaceutically acceptable salt or deuterated form thereof, wherein R¹ is

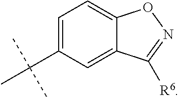

116. The compound of any one of clauses 100-115, or a pharmaceutically acceptable salt or deuterated form thereof, wherein R⁶ is methyl.

117. The compound of any one of clauses 100-115, or a pharmaceutically acceptable salt or deuterated form thereof, wherein R⁶ is ethyl.

118. The compound of any one of clauses 100-115, or a pharmaceutically acceptable salt or deuterated form thereof, wherein R⁶ is propyl.

119. The compound of any one of clauses 1-107, or a pharmaceutically acceptable salt or deuterated form thereof, wherein R¹ is

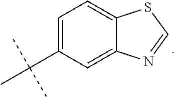

120. The compound of any one of clauses 1-107, or a pharmaceutically acceptable salt or deuterated form thereof, wherein R¹ is

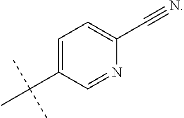

121. The compound of any one of clauses 1-107, or a pharmaceutically acceptable salt or deuterated form thereof, wherein R¹ is

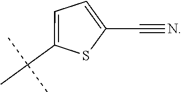

122. The compound of any one of clauses 1-74, wherein the compound is of Formula (VIII):

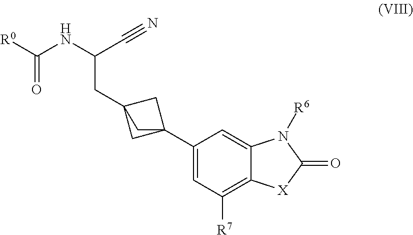

or a pharmaceutically acceptable salt or deuterated form thereof.

123. The compound of any one of clauses 1-74, wherein the compound is of Formula (IX),

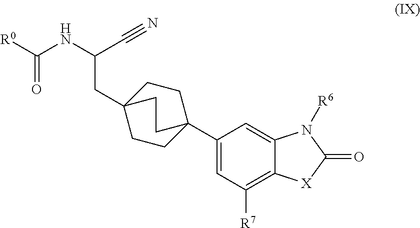

or a pharmaceutically acceptable salt or deuterated form thereof.

124. The compound of any one of clauses 1-74, wherein the compound is of Formula (X),

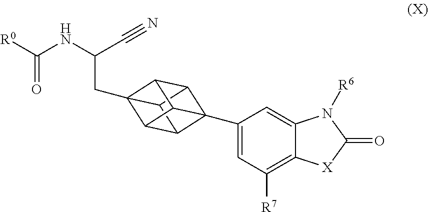

or a pharmaceutically acceptable salt or deuterated form thereof.

125. The compound of any one of clauses 1-69, wherein the compound is of Formula (XI),

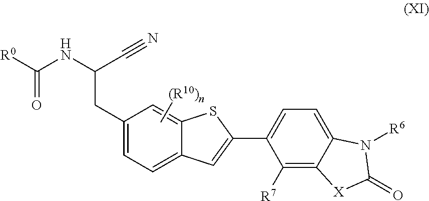

or a pharmaceutically acceptable salt or deuterated form thereof,
wherein
   n is 0 or 1, and
   R¹⁰ can be substituted on the phenyl ring or the thiophenyl ring of the benzothienylene ring.

126. The compound of any one of clauses 1-69, wherein the compound is of Formula (XII),

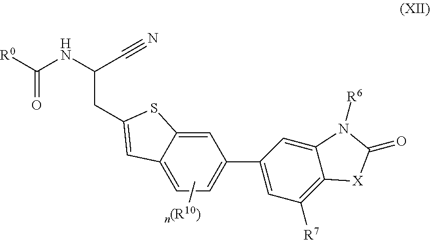

or a pharmaceutically acceptable salt or deuterated form thereof,
wherein
   n is 0 or 1, and
   R¹⁰ can be substituted on the phenyl ring or the thiophenyl ring of the benzothienylene ring.

127. The compound of any one of clauses 1-69, wherein the compound is of Formula (XIII),

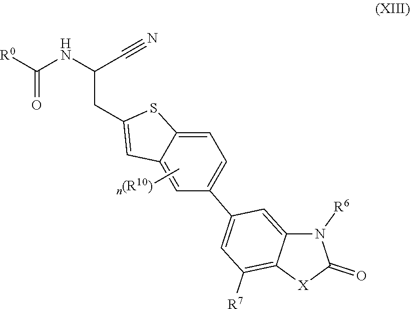

or a pharmaceutically acceptable salt or deuterated form thereof,
wherein
   n is 0 or 1, and
   R¹⁰ can be substituted on the phenyl ring or the thiophenyl ring of the benzothienylene ring.

128. The compound of any one of clauses 1-69, wherein the compound is of Formula (XVI),

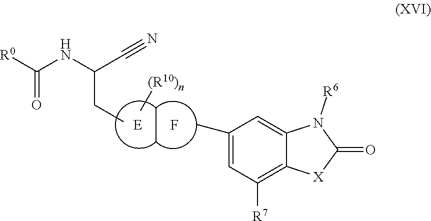

or a pharmaceutically acceptable salt or deuterated form thereof,
wherein
   each of ring E and Ring F is 5-membered heteroarylene,
   n is 0 or 1, and
   R¹⁰ is substituted on the ring E or ring F.

129. The compound of any one of clauses 1-69, wherein the compound is of Formula (XVII),

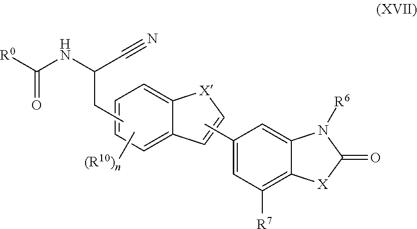

or a pharmaceutically acceptable salt or deuterated form thereof,
wherein
   X′ is O, S, NH or N(C₁₋₆ alkyl),
   n is 0 or 1, and
   R¹⁰ is substituted on the phenyl ring or the 5-membered ring of

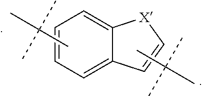

130. The compound of any one of clauses 1-69, wherein the compound is of Formula (XVIII),

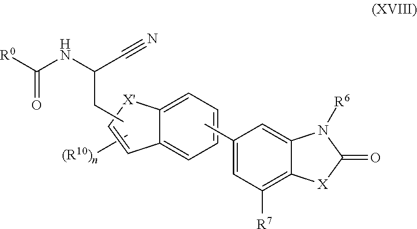

or a pharmaceutically acceptable salt or deuterated form thereof,
wherein
   X′ is O, S, NH or N(C₁₋₆ alkyl),
   n is 0 or 1, and
   R¹⁰ is substituted on the phenyl ring or the 5-membered ring of

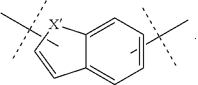

131. The compound of any one of clauses 122-129, or a pharmaceutically acceptable salt or deuterated form thereof, wherein n is 0.

132. The compound of any one of clauses 122-129, or a pharmaceutically acceptable salt or deuterated form thereof, wherein n is 1 and R¹⁰ is halo.

133. The compound of any one of clauses 122-129 or 132, or a pharmaceutically acceptable salt or deuterated form thereof, wherein n is 1 and R¹⁰ is F.

134. The compound of any one of clauses 122-133, or a pharmaceutically acceptable salt or deuterated form thereof, wherein X is O.

135. The compound of any one of clauses 122-134, or a pharmaceutically acceptable salt or deuterated form thereof, wherein R⁶ is C₁₋₃ alkyl.

136. The compound of any one of clauses 122-135, or a pharmaceutically acceptable salt or deuterated form thereof, wherein R⁷ is H.

137. The compound of any one of clauses 122-136, or a pharmaceutically acceptable salt or deuterated form thereof, wherein X is O, R⁶ is C₁₋₃ alkyl and R⁷ is H.

138. The compound of any one of clauses 1-137, or a pharmaceutically acceptable salt or deuterated form thereof, wherein R⁰ is

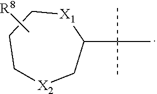

139. The compound of any one of clauses 1-138, or a pharmaceutically acceptable salt or deuterated form thereof, wherein R⁰ is

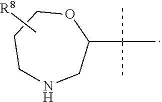

140. The compound of any one of clauses 138-139, wherein R⁸ is H.

141. The compound of any one of clauses 1-137, or a pharmaceutically acceptable salt or deuterated form thereof, wherein R⁰ is

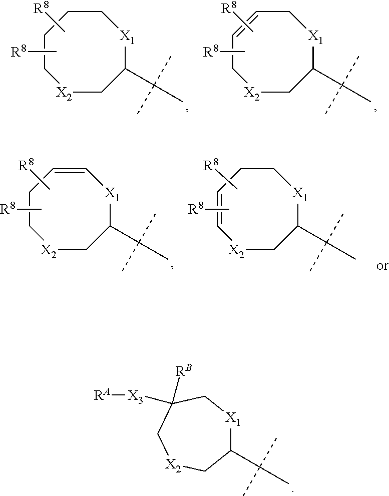

142. The compound of any one of clauses 1 or 141, or a pharmaceutically acceptable salt or deuterated form thereof, wherein R⁰ is

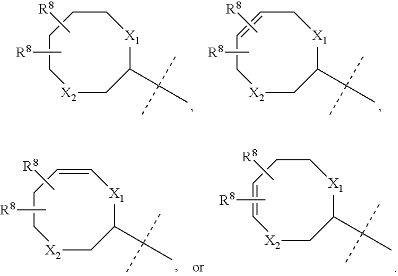

143. The compound of clause 142, or a pharmaceutically acceptable salt or deuterated form thereof, wherein R⁰ is

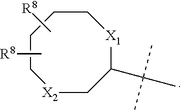

144. The compound of clause 142, or a pharmaceutically acceptable salt or deuterated form thereof, wherein R⁰ is

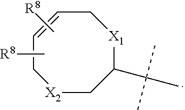

145. The compound of clause 142, or a pharmaceutically acceptable salt or deuterated form thereof, wherein R⁰ is

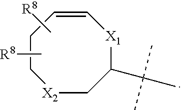

146. The compound of clause 142, or a pharmaceutically acceptable salt or deuterated form thereof, wherein R⁰ is

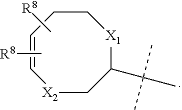

147. The compound of any one of clauses 1 or 141-146, or a pharmaceutically acceptable salt or deuterated form thereof, wherein at least one R⁸ is H.

148. The compound of any one of clauses 1 or 141-146, or a pharmaceutically acceptable salt or deuterated form thereof, wherein at least one R⁸ is methoxy.

149. The compound of any one of clauses 1 or 141-146, or a pharmaceutically acceptable salt or deuterated form thereof, wherein at least one R⁸ is ethoxy.

150. The compound of any one of clauses 1 or 131-136, or a pharmaceutically acceptable salt or deuterated form thereof, wherein at least one R⁸ is OH.

151. The compound of any one of clauses 1-140, or a pharmaceutically acceptable salt or deuterated form thereof, wherein R⁰ is

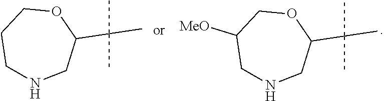

152. The compound of any one of clauses 1-140 or 151, or a pharmaceutically acceptable salt or deuterated form thereof, wherein R⁰ is

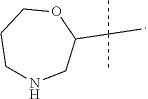

153. The compound of any one of clauses 1-140 or 151, or a pharmaceutically acceptable salt or deuterated form thereof, wherein R⁰ is

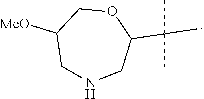

154. The compound of clause 141 or 142, or a pharmaceutically acceptable salt or deuterated thereof, wherein R⁰ is

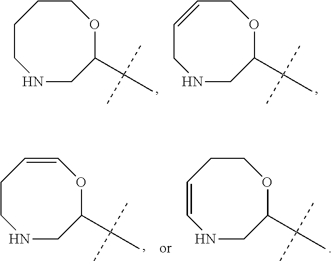

155. The compound of clause 154, or a pharmaceutically acceptable salt or deuterated thereof, wherein R⁰ is

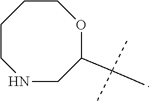

156. The compound of clause 154, or a pharmaceutically acceptable salt or deuterated form/thereof, wherein R⁰ is

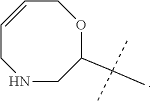

157. The compound of clause 154, or a pharmaceutically acceptable salt or deuterated form thereof, wherein R⁰ is

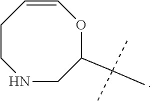

158. The compound of clause 154, or a pharmaceutically acceptable salt or deuterated form thereof, wherein R⁰ is

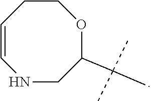

159. The compound of any one of clauses 1-137 or 141, or a pharmaceutically acceptable salt or deuterated form thereof, wherein R⁰ is

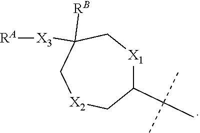

160. The compound of clause 159, or a pharmaceutically acceptable salt or deuterated form thereof, wherein R⁰ is

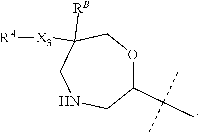

161. The compound of any one of clauses 140 or 159-160, or a pharmaceutically acceptable salt or deuterated form thereof, wherein R^{A} is H or C₁₋₆alkyl.

162. The compound of clause 161, or a pharmaceutically acceptable salt or deuterated form thereof, wherein R^{A} is H.

163. The compound of clause 161, or a pharmaceutically acceptable salt or deuterated form thereof, wherein R^{A} is methyl.

164. The compound of any one of clauses 141 or 159-163, or a pharmaceutically acceptable salt or deuterated form thereof, wherein R^{B} is C₁₋₆alkyl, C₂₋₆ alkenyl, C₁₋₆alkylene-carbocyclyl, or C₁₋₆alkylene-heteroaryl.

165. The compound of clause 164, or a pharmaceutically acceptable salt or deuterated form thereof, wherein R^{B} is C₁₋₆alkyl, C₁₋₆alkylene-aryl or —C₁₋₆alkylene-5-6 membered heteroaryl.

166. The compound of clause 159 or 160, or a pharmaceutically acceptable salt or deuterated form thereof, wherein R⁰ is

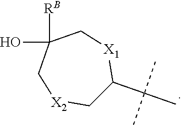

167. The compound of clause 159 or 160, or a pharmaceutically acceptable salt or deuterated form thereof, wherein R⁰ is

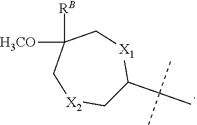

168. The compound of clause 159 or 160, or a pharmaceutically acceptable salt or deuterated form thereof, wherein R⁰ is

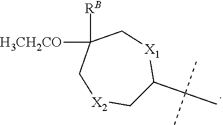

169. The compound of any one of clauses 164-168, or a pharmaceutically acceptable salt or deuterated form thereof, wherein R^{B} is methyl.

170. The compound of any one of clauses 164-168, or a pharmaceutically acceptable salt or deuterated form thereof, wherein R^{B} is ethyl.

171. The compound of any one of clauses 166-168, wherein R^{B} is H.

172. The compound of clause 167, or a pharmaceutically acceptable salt or deuterated form thereof, wherein R⁰ is

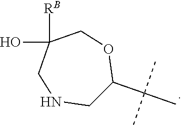

173. The compound of clause 168, or a pharmaceutically acceptable salt or deuterated form thereof, wherein R⁰ is

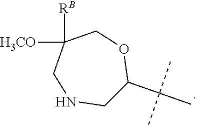

174. The compound of clause 169, or a pharmaceutically acceptable salt or deuterated form thereof, wherein R⁰ is

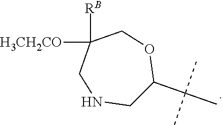

175. The compound of any one of clauses 172-174, or a pharmaceutically acceptable salt or deuterated form thereof, wherein R^{B} is methyl.

176. The compound of any one of clauses 172-174, or a pharmaceutically acceptable salt or deuterated form thereof, wherein R^{B} is ethyl.

177. The compound of any one of clauses 159-165, or a pharmaceutically acceptable salt or deuterated form thereof, wherein X₃ is O.

178. The compound of any one of clauses 1-137, or a pharmaceutically acceptable salt or deuterated form thereof, wherein R⁰ is

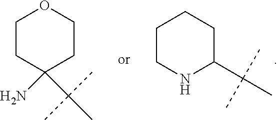

179. The compound of any one of clauses 122-178, or a pharmaceutically acceptable salt or deuterated form thereof, wherein R⁶ is C₁₋₃ alkyl.

180. The compound of clause 179, or a pharmaceutically acceptable salt or deuterated form thereof, wherein R⁶ is CH₃.

181. The compound of any one of clauses 122-180, or a pharmaceutically acceptable salt or deuterated form thereof, wherein R⁷ is H.

182. The compound of any one of clauses 122-181, or a pharmaceutically acceptable salt or deuterated form thereof, wherein R¹⁰ is —H or —F.

183. The compound of clause 1, or a pharmaceutically acceptable salt or deuterated form thereof, wherein the compound is selected from Table 1.

184. A pharmaceutical composition comprising an effective amount of a compound of any one of clauses 1-183, or a pharmaceutically acceptable salt or deuterated form thereof and a pharmaceutically acceptable adjuvant, diluent or carrier.

185. A method for treating an obstructive disease of the airway in a patient in need thereof, comprising, administering to the patient an effective amount of a compound of any one of clauses 1-183, or the composition of clause 184.

186. The method of clause 185, wherein the obstructive disease of the airway is asthma, chronic obstructive pulmonary disease (COPD), bronchitis, emphysema, cystic fibrosis (CF), bronchiectasis, sarcoidosis, alpha-1 antitrypsin (A1AT) deficiency, farmer's lung and related diseases, hypersensitivity pneumonitis, lung fibrosis, complications of lung transplantation, vasculitic and thrombotic disorders of the lung vasculature, pulmonary hypertension, antitussive activity including treatment of chronic cough associated with inflammatory and secretory conditions of the airways, iatrogenic cough, acute and chronic rhinitis including rhinitis medicamentosa, and vasomotor rhinitis; perennial and seasonal allergic rhinitis including rhinitis nervosa (hay fever), nasal polyposis; acute viral infection including the common cold, and infection due to a respiratory virus, acute lung injury, or acute respiratory distress syndrome (ARDS).

187. The method of clause 186, wherein the obstructive disease of the airway is asthma.

188. The method of clause 186, wherein the obstructive disease of the airway is acute respiratory distress syndrome (ARDS).

189. The method of clause 186, wherein the obstructive disease of the airway is bronchitis.

190. The method of clause 186, wherein the obstructive disease of the airway is lung fibrosis.

191. The method of clause 186, wherein the obstructive disease of the airway is emphysema.

192. The method of clause 186, wherein the obstructive disease of the airway is cystic fibrosis (CF).

193. The method of clause 186, wherein the obstructive disease of the airway is bronchiectasis.

194. The method of clause 186, wherein the obstructive disease of the airway is sarcoidosis.

195. The method of clause 186, wherein the obstructive disease of the airway is alpha-1 antitrypsin (A1AT) deficiency.

196. The method of clause 186, wherein the obstructive disease of the airway is farmer's lung.

197. The method of clause 186, wherein the obstructive disease of the airway is hypersensitivity pneumonitis.

198. The method of clause 186, wherein the obstructive disease of the airway is a complication of lung transplantation.

199. The method of clause 186, wherein the obstructive disease of the airway is a vasculitic or thrombotic disorder of the lung vasculature.

200. The method of clause 186, wherein the obstructive disease of the airway is pulmonary hypertension.

201. The method of clause 186, wherein the obstructive disease of the airway is iatrogenic cough.

202. The method of clause 186, wherein the obstructive disease of the airway is acute rhinitis.

203. The method of clause 186, wherein the obstructive disease of the airway is chronic rhinitis.

204. The method of clause 186, wherein the obstructive disease of the airway is rhinitis medicamentosa or vasomotor rhinitis.

205. The method of clause 186, wherein the obstructive disease of the airway is nasal polyposis.

206. The method of clause 186, wherein the obstructive disease of the airway is COPD.

207. The method of clause 187, wherein the asthma is bronchial, allergic, intrinsic, extrinsic, exercise-induced or drug-induced asthma.

208. The method of clause 187, wherein the bronchitis is infectious bronchitis or eosinophilic bronchitis.

209. The method of clause 190, wherein the lung fibrosis is idiopathic pulmonary fibrosis, cryptogenic fibrosing alveolitis, idiopathic interstitial pneumonia, or fibrosis complicating anti-neoplastic therapy or chronic infection.

210. The method of clause 193, wherein the bronchiectasis is non-cystic fibrosis bronchiectasis (NCFBE).

211. The method of clause 193, wherein the bronchiectasis is associated with cystic fibrosis.

212. The method of clause 200, wherein the pulmonary hypertension is pulmonary arterial hypertension.

213. The method of clause 200, wherein the pulmonary hypertension is pulmonary hypertension due to left heart disease.

214. The method of clause 200, wherein the pulmonary hypertension is pulmonary hypertension associated with chronic lung disease.

215. A method for treating cystic fibrosis in a patient in need thereof, comprising, administering to the patient an effective amount of a compound of any one of clauses 1-183, or the composition of clause 184.

216. The method of clause 215, wherein the treating comprises improving the lung function of the patient, as compared to the lung function of the patient prior to treatment.

217. The method of clause 216, wherein improving lung function of the patient comprises increasing the patient's forced expiratory volume in 1 second (FEV₁), increasing the patient's forced vital capacity (FVC), increasing the patient's peak expiratory flow rate (PEFR), or increasing the patient's forced expiratory flow between 25% and 75% of FVC (FEF_{(25-75%)}), as compared to the respective value for the patient prior treatment.

218. The method of clause 216 or 217, wherein the lung function is measured by spirometry.

219. A method for treating bronchiectasis in a patient in need thereof, comprising, administering to the patient, an effective amount of a compound of any one of clauses 1-183, or the composition of clause 184.

220. The method of clause 219, wherein the bronchiectasis is non-cystic fibrosis bronchiectasis (NCFBE).

221. The method of clause 219, wherein the bronchiectasis is associated with cystic fibrosis.

222. The method of any one of clauses 219-221, wherein treating comprises improving the lung function of the patient, as compared to the lung function of the patient prior to treatment.

223. The method of clause 222, wherein improving lung function of the patient comprises increasing the patient's forced expiratory volume in 1 second (FEV₁), increasing the patient's forced vital capacity (FVC), increasing the patient's peak expiratory flow rate (PEFR), or increasing the patient's forced expiratory flow between 25% and 75% of FVC (FEF_{(25-75%)}), as compared to the respective value for the patient prior to treatment.

224. The method of clause 222 or 223, wherein the lung function is measured by spirometry.

225. The method of any one of clauses 219-224, wherein treating comprises decreasing the rate of pulmonary exacerbation, as compared to the rate of pulmonary exacerbation of the patient prior to treatment.

226. The method of any one of clauses 219-225, wherein treating comprises increasing the time to first pulmonary exacerbation, as compared to an untreated patient.

227. The method of clause 225 or 226, wherein the pulmonary exacerbation is characterized by three or more of the following symptoms exhibited for at least 48 hours by the patient: (1) increased cough; (2) increased sputum volume or change in sputum consistency; (3) increased sputum purulence; (4) increased breathlessness and/or decreased exercise tolerance; (5) fatigue and/or malaise; (6) hemoptysis.

228. A method for treating chronic rhinosinusitis (CRS) in a patient in need thereof, comprising, administering to the patient, an effective amount of a compound of any one of clauses 1-183, or the composition of clause 184.

229. The method of clause 228, wherein the chronic rhinosinusitis is chronic rhinosinusitis without nasal polyps (CRSsNP).

230. The method of clause 228, wherein the chronic rhinosinusitis is chronic rhinosinusitis with nasal polyps (CRSwNP).

231. The method of any one of clauses 228-229, wherein the chronic rhinosinusitis is refractory chronic rhinosinusitis.

232. The method of any one of clauses 228-231, wherein treating comprises reducing, diminishing the severity of, delaying the onset of, or eliminating one or more symptoms of CRS.

233. The method of clause 232, wherein the one or more symptoms of CRS is nasal congestion; nasal obstruction; nasal discharge; post-nasal drip; facial pressure; facial pain; facial fullness; reduced smell; depression; mucosal edema; mucopurulent discharge; obstruction of the middle meatus; mucosal changes within the ostiomeatal complex and sinuses; or rhinorrhea.

234. A method for treating hidradenitis suppurativa (HS) in a patient in need thereof, comprising, administering to the patient, an effective amount of a compound of any one of clauses 1-183, or the composition of clause 184.

235. The method of clause 234, wherein the hidradenitis suppurativa (HS) is Hurley stage I.

236. The method of clause 234, wherein the hidradenitis suppurativa (HS) is Hurley stage II.

237. The method of clause 234, wherein the hidradenitis suppurativa (HS) is Hurley stage III.

238. A method for treating cancer in a patient in need thereof, comprising, administering to the patient, an effective amount of a compound of any one of clauses 1-183, or the composition of clause 184.

239. The method of clause 238, wherein the cancer is a metastatic cancer.

240. The method of clause 239, wherein the metastatic cancer is breast to lung metastatic cancer.

241. The method of clause 239, wherein the metastatic cancer comprises metastasis of breast cancer to the brain, bone, pancreas, lymph nodes or liver.

242. The method of clause 239, wherein the metastatic cancer comprises metastasis of bone cancer to the lung.

243. The method of clause 239, wherein the metastatic cancer comprises metastasis of colorectal cancer to the peritoneum, the pancreas, the stomach, the lung, the liver, the kidney, or the spleen.

244. The method of clause 239, wherein the metastatic cancer comprises metastasis of stomach cancer to the mesentery, the spleen, the pancreas, the lung, the liver, the adrenal gland, or the ovary.

245. The method of clause 239, wherein the metastatic cancer comprises metastasis of liver cancer to the intestine, spleen, pancreas, stomach, lung, or the kidney.

246. The method of clause 239, wherein the metastatic cancer comprises metastasis of lymphoma to the kidney, ovary, liver, bladder, or the spleen.

247. A method for treating lupus nephritis in a patient in need thereof, comprising, administering to the patient, an effective amount of a compound of any one of clauses 1-183, or the composition of clause 184.

248. A method for treating rheumatoid arthritis in a patient in need thereof, comprising, administering to the patient, an effective amount of a compound of any one of clauses 1-183, or the composition of clause 184.

249. A method for treating inflammatory bowel disease (IBD) in a patient in need thereof, comprising, administering to the patient, an effective amount of a compound of any one of clauses 1-183, or the composition of clause 184.

250. The method of clause 249, wherein the inflammatory bowel disease (IBD) is Crohn's disease.

251. The method of clause 249, wherein the inflammatory bowel disease (IBD) is ulcerative colitis.

252. A method for treating an anti-neutrophil cytoplasmic antibody (ANCA) associated vasculitis in a patient in need thereof, comprising, administering to the patient, an effective amount of a compound of any one of clauses 1-183, or the composition of clause 184.

253. The method of clause 252, wherein the ANCA associated disease is granulomatosis with polyangiitis (GPA).

254. The method of clause 252, wherein the ANCA associated disease is microscopic polyangiitis (MPA).

255. A method for treating a disease in a patient in need thereof comprising, administering to the patient, an effective amount of a compound of any one of clauses 1-183, or the composition of clause 184, wherein the disease is giant cell arteritis, polyarteritis nodosa, anti-GBM disease (Goodpasture's), systemic scleroderma, diabetic nephropathy, diabetic neuropathy, diabetic retinopathy, diabetic ulcers, Duchenne muscular dystrophy, bronchiolitis obliterans, atopic dermatitis, pyoderma gangrenosum, sweet's syndrome, dermatomyositis/polymyositis, neutrophilic dermatoses, thrombosis, bronchopulmonary dysplasia, amyotrophic lateral sclerosis, sickle cell anemia, psoriasis, or a ventilator-induced lung injury.

256. A method for treating a heart failure in a patient in need thereof, comprising administering to the patient, an effective amount of a compound of any one of clauses 1-183, or the composition of clause 184.

257. The method of clause 256, wherein the heart failure is heart failure with reduced ejection fraction.

258. The method of clause 256, wherein the heart failure is heart failure with preserved ejection fraction.

259. A compound selected from Table 1, or a pharmaceutically acceptable salt or deuterated form thereof.

1. A compound of formula (I) 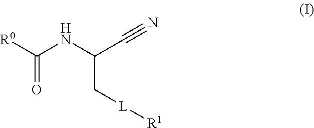
or a pharmaceutically acceptable salt or deuterated form thereof,
wherein:
R⁰ is
5-12 membered monocyclic heterocyclyl containing 1-3 heteroatoms selected from N, S, or O, wherein the monocyclic heterocycle is optionally substituted with 1-3 R⁸, or
5-12-membered polycyclic heterocyclyl containing 1-3 heteroatoms selected from N, S, or O, wherein the polycyclic heterocycle is optionally substituted with 1-3 R⁸,
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy, halogenated C₁₋₆alkoxy, C₁₋₆ alkylene-carbocyclyl, or C₁₋₆ alkylene-heteroaryl;
L is polycyclic cycloalkylene, polycyclic arylene, or polycyclic heteroarylene, wherein a first atom of a first ring of the polycyclic arylene or heteroarylene is connected to 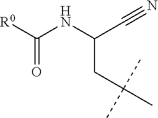
and a second atom of a second ring of the polycyclic arylene or heteroarylene is connected to R¹;
wherein L is independently substituted by 0-4 R¹⁰;
each R¹⁰ is independently ═O, halogen, C₁₋₆alkyl, C₁₋₆alkoxy, S—C₁₋₆alkyl, C₂₋₆alkenyl, C₂₋₆alkynyl, C₃₋₆cycloalkyl, cyano, hydroxy, NH₂, —NH—C₁₋₆ alkyl, N(C₁₋₆alkyl)₂, COOH, COC₁₋₆ alkyl, COOC₁₋₆alkyl, CON₁₋₆alkyl, CON(C₁₋₆alkyl)₂, NHCOC₁₋₆alkyl, or heterocycle; wherein each alkyl, alkoxy, alkenyl, alkynyl, cycloalkyl, and heterocycle are independently optionally substituted with 1-3 substituents selected from halogen, cyano, hydroxyl, NH₂ and COOH;
R¹ is
5-12 membered carbocyclyl optionally substituted with 1-3 R^{g},
6-18 membered aryl optionally substituted with 1-3 R^{g},
5-12 membered monocyclic heterocyclyl containing 1-3 heteroatoms selected from N, S or O, wherein the monocyclic heterocyclyl is optionally substituted with 1-3 R^{g},
5-12 membered monocyclic heteroaryl containing 1-3 heteroatoms selected from N, Sor O, wherein the monocyclic heteroaryl is optionally substituted with 1-3 R^{g},
7-14 membered bicyclic heteroaryl containing 1-3 heteroatoms selected from N, S or O, wherein the bicyclic heteroaryl is optionally substituted with 1-4 R^{g}, or
7-20 membered tricyclic heteroaryl containing 1-3 heteroatoms selected from N, S or O, wherein the tricyclic heteroaryl is optionally substituted with 1-5 R^{g},
each R^{g} is independently selected from hydrogen, SF₅, ═O, halogen, cyano, hydroxyl, nitro, NH₂, —COOH, C₁₋₆ alkyl, C₁₋₆ alkoxy, C₃₋₆ cycloalkyloxy, C₂₋₆alkenyloxy, C₂₋₆ alkynyloxy, C₃₋₆ cycloalkyl, C₂₋₆ alkenyl, C₂₋₅ alkynyl, —NHC₁₋₆ alkyl, —N(C₁₋₆ alkyl)₂, —COC₁₋₆alkyl, —COOC₁₋₆ alkyl, —CONH₂, —CONHC₁₋₆ alkyl, —CONHC₃₋₆ cycloalkyl, —CON(C₁₋₆ alkyl)₂, —NHCOC₁₋₆alkyl, —NHCOC₃₋₅cycloalkyl, —P(O)(C₁₋₆ alkyl), —S(O)C₁₋₆alkyl, —S(O)₂C₁₋₆ alkyl, —S(O)₂C₃₋₆cycloalkyl, —SO₂-3-7 membered heterocyclyl, —S(O)NH₂, —S(O)NHC₁₋₆ alkyl, —S(O)N(C₁₋₆ alkyl)₂, —S(O)₂N—H₂, —OSO₂—C₁₋₆alkyl, C₁₋₆alkylene-O—C₁₋₆alkyl, C₃₋₈-cycloalkenyloxy, aryl, heteroaryl, 3-7-membered heterocycle containing 1-3 heteroatoms selected from N, S and O, and the R^{g} is optionally further substituted with 1-3 groups selected from C₁₋₆ alkyl, C₁₋₆ haloalkyl, C₁₋₆-alkylene-OH, —CONH₂, NH₂, C₁₋₆ alkoxy, hydroxyl, —COOH, halogen, or a 5-7-membered heterocycle containing 1-3 heteroatoms selected from N, S or O, wherein the 5-7-membered heterocycle is optionally substituted with 1-2 groups selected from ═O, halogen, cyano, C₁₋₆ alkyl, and C₁₋₆ haloalkyl.

2. The compound of claim 1, or a pharmaceutically acceptable salt or deuterated form thereof, wherein R⁰ is 5-12 membered monocyclic heterocycle containing 1-3 heteroatoms selected from N, S, or O, wherein the monocyclic heterocycle is optionally substituted with 1-3 R⁸.

3. The compound of claim 1 or 2, or a pharmaceutically acceptable salt or deuterated form thereof, wherein R⁰ is 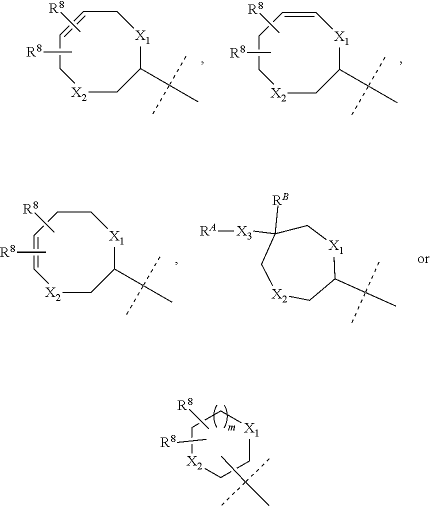
X₁ and X₂ are independently O, S, NH, N(C₁₋₆alkyl), or CR¹²R¹³, wherein at least one of X₁ and X₂ are not CR¹²R¹³;
X₃ is O, S, NH, or N(C₁₋₆alkyl);
R⁸ is H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy or halogenated C₁₋₆alkoxy;
R¹² is H, halogen, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy or halogenated C₁₋₆alkoxy
R¹³ is independently H, halogen or C₁-C₆ alkyl;
R^{A} is H, C₁₋₆alkyl, C₁₋₆alkylene-carbocyclyl, or C₁₋₆alkylene-heteroaryl; and
R^{B} is C₁₋₆alkyl, C₂₋₆alkenyl, C₁₋₆alkylene-carbocyclyl, or C₁₋₆alkylene-heteroaryl, or R^{A} and R^{B} are taken together to form a heterocyclyl; and
m is 0, 1, 2 or 3.

4. The compound of claim 3, or a pharmaceutically acceptable salt or deuterated form thereof, wherein R⁰ is 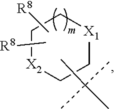
and m is 2 or 3.

5. The compound of claim 3 or 4, or a pharmaceutically acceptable salt or deuterated form thereof, wherein R⁰ is 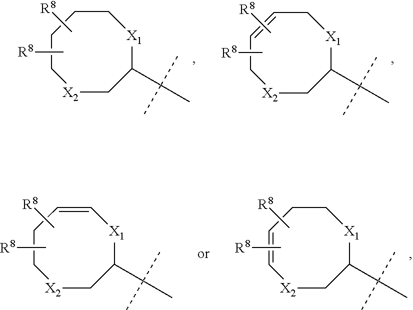
X₁ and X₂ are independently O, S, NH, N(C₁₋₆alkyl), or CR¹²R¹³, wherein at least one of X₁ and X₂ are not CR¹²R¹³;
each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy or halogenated C₁₋₆alkoxy; and
R¹² is H, halogen, C₁₋₆ alkyl; and
each R¹³ is independently H, halogen or C₁-C₆ alkyl.

6. The compound of claim 3, or a pharmaceutically acceptable salt or deuterated form thereof, wherein R⁰ is 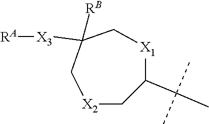
X₁ and X₂ are independently O, S, NH, N(C₁₋₆alkyl), or CR¹²R¹³, wherein at least one of X₁ and X₂ are not CR¹²R¹³;
X₃ is O, S, NH, or N(C₁₋₆alkyl);
R^{A} is H, C₁₋₆alkyl, C₁₋₆alkylene-carbocyclyl, or C₁₋₆alkylene-heteroaryl; and
R^{B} is C₁₋₆alkyl, C₂₋₆alkenyl, C₁₋₆alkylene-carbocyclyl, or C₁₋₆alkylene-heteroaryl; or
R^{A} and R^{B} are taken together to form a heterocyclyl;
R¹² is H, halogen, C₁₋₆ alkyl;
each R¹³ is independently H, halogen or C₁-C₆ alkyl.

7. The compound of claim 5, or a pharmaceutically acceptable salt or deuterated form thereof, wherein R⁰ is 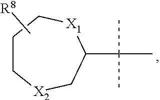
X₁ is O, X₂ is NH, and R⁸ is C₁₋₆alkoxy.

8. The compound of claim 1 or 2, wherein R⁰ is 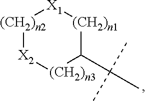
each n1, n2 and n3 is independently an integer from 0-3, and the total sum of n1, n2 and n3 is ≤4, X₁ and X₂ are independently O, S, NR⁶ or CR¹²R¹³, wherein at least one of the X₁ and X₂ is not CR¹²R¹³; each R⁶, R¹² and R¹³ is independently H, halo, or C₁-C₆ alkyl.

9. The compound of any one of claim 1-8, or a pharmaceutically acceptable salt or deuterated form thereof, wherein R⁰ is 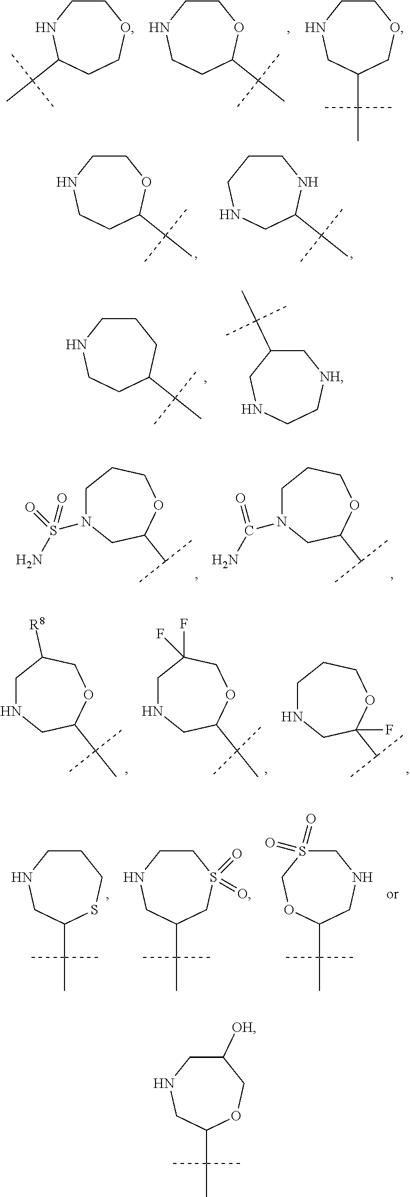
wherein R⁸ is H, F, OH, CH₃, OCH₃, OCHF₂, OCF₃, OCH₂CH₃, or —CH₂OCH₃.

10. The compound of any one of claims 1-4, wherein R⁰ is

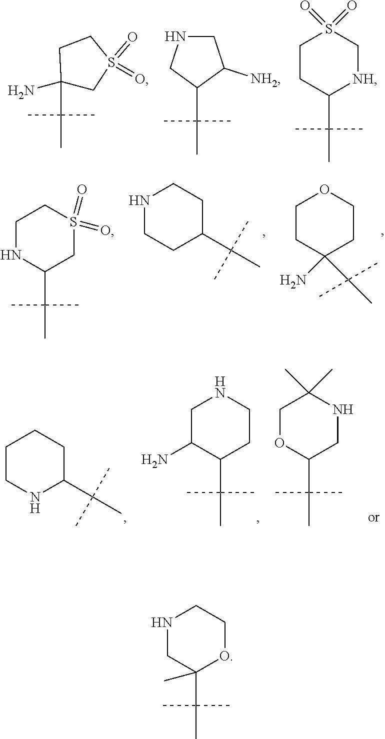

11. The compound of claim 1, or a pharmaceutically acceptable salt or deuterated form thereof, wherein R⁰ is 5-12-membered polycyclic heterocycle containing 1-3 heteroatoms selected from N, S, O, wherein the polycyclic heterocycle is optionally substituted with 1-3 R⁸.

12. The compound of claim 1 or 11, or a pharmaceutically acceptable salt or deuterated form thereof, wherein R⁰ is
5-12 membered spiro heterocycle containing 1-3 heteroatoms selected from N, S, or O, wherein the spiro heterocycle is optionally substituted with 1-3 R⁸,
5-12 membered fused heterocycle containing 1-3 heteroatoms selected from N, S, or O, wherein the spiro heterocycle is optionally substituted with 1-3 R⁸, or
7-12 membered bridged heterocycle containing 1-3 heteroatoms selected from N, S, or O, wherein the bridge heterocycle is optionally substituted with 1-3 R⁸.

13. The compound of any one of claims 1, 11, or 12, or a pharmaceutically acceptable salt or deuterated form thereof, wherein R⁰ is 5-12 membered spiro heterocycle containing 1-3 heteroatoms selected from N, S, or O, wherein the spiro heterocycle is optionally substituted with 1-3 R⁸.

14. The compound of claim 12 or 13, or a pharmaceutically acceptable salt or deuterated form thereof, wherein R⁰ is 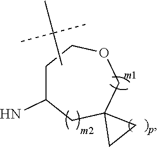
wherein
m1 and m2 are each independently 0, 1, or 2, provided that both m1 and m2 are not 0, or both m1 and m2 are not 2, and
p is 1 or 2.

15. The compound of any one of claims 12-14, or a pharmaceutically acceptable salt or deuterated form thereof, wherein R⁰ is

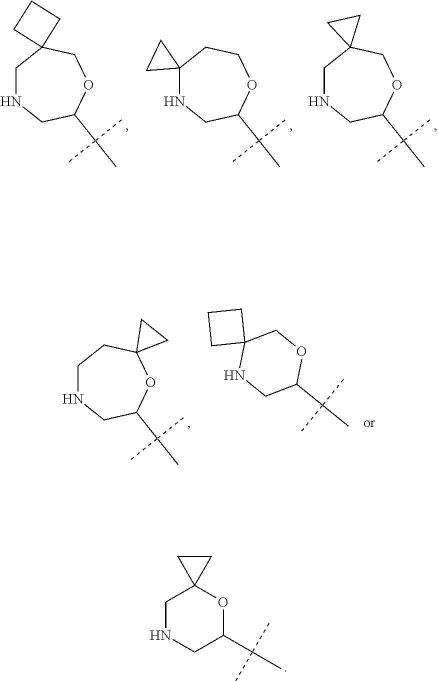

16. The compound of claim 1, 11, or 12, or a pharmaceutically acceptable salt or deuterated form thereof, wherein R⁰ is 5-12 membered fused heterocycle containing 1-3 heteroatoms selected from N, S, O, wherein the fused heterocycle is optionally substituted with 1-3 R⁸.

17. The compound of claim 16, or a pharmaceutically acceptable salt or deuterated form thereof, wherein R⁰ is 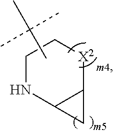
m4 is 0 or 1,
m5 is 1 or 2,
X² is O, S, NH, N(C₁₋₆alkyl), or CR¹²R¹³,
R¹² is selected from H, halogen, cyano, hydroxyl, NH₂, NH(C₁₋₆alkyl), N(C₁₋₆alkyl)₂, COOH, C₁₋₆ alkyl, —CONH₂, —S(═O)NH₂, —S(O)₂NH₂, C₁₋₆alkoxy or halogenated C₁₋₆alkoxy;
R¹³ is independently H, F, Cl, Br, I or C₁-C₆ alkyl.

18. The compound of claim 16, or a pharmaceutically acceptable salt or deuterated form thereof, wherein R⁰ is 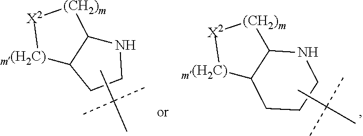
X₂ is O, S, NH, N(C₁₋₆alkyl), or CR¹²R¹³
R¹² is H, halogen, cyano, hydroxyl, NH₂, NH(C₁₋₆alkyl), N(C₁₋₆alkyl)₂, COOH, C₁₋₆ alkyl, —CONH₂, —S(═O)NH₂, —S(O)₂NH₂, C₁₋₆alkoxy or halogenated C₁₋₆alkoxy;
R¹³ is H, halogen or C₁-C₆ alkyl;
each m and m′ is independently an integer from 0-3, and the total sum of m and m′ is ≤3.

19. The compound of claim 17, or a pharmaceutically acceptable salt or deuterated form thereof, wherein R⁰ is

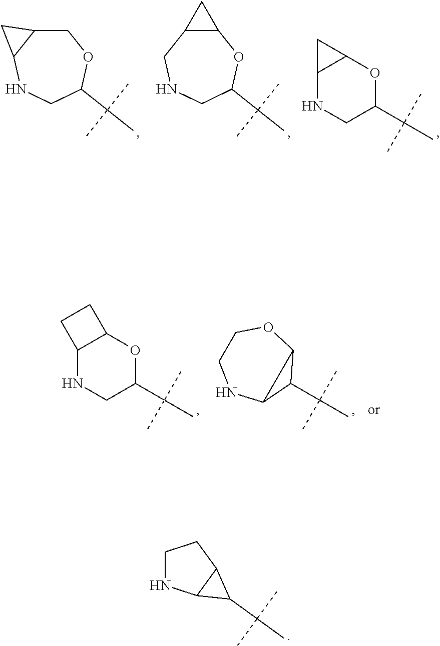

20. The compound of claim 12 or 16, or a pharmaceutically acceptable salt or deuterated form thereof, wherein R⁰ is 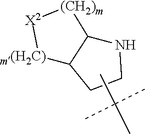
X₂ is O, S, NH, N(C₁₋₆alkyl), or CR¹²R¹³,
R¹² is H, halogen, C₁₋₆ alkyl,
R¹³ is H, halogen or C₁-C₆ alkyl, and
each m and m′ is independently an integer from 0-3, and the total sum of me and mc′ is ≤3.

21. The compound of claim 20, or a pharmaceutically acceptable salt or deuterated form thereof, wherein R⁰ is 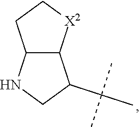
X² is NH, O, or S.

22. The compound of claim 21, or a pharmaceutically acceptable salt or deuterated form thereof, wherein R⁰ is

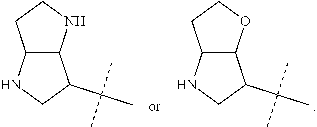

23. The compound of claim 20, or a pharmaceutically acceptable salt or deuterated form thereof, the wherein R⁰ is

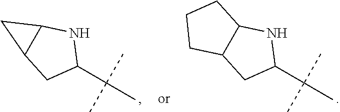

24. The compound of claim 18, or a pharmaceutically acceptable salt or deuterated form thereof, the wherein R⁰ is 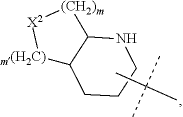
X₂ is O, S, NH, N(C₁₋₆alkyl), or CR¹²R¹³
R¹² is H, halogen, C₁₋₆ alkyl; and
R¹³ is H, halogen or C₁-C₆ alkyl.

25. The compound of claim 24, or a pharmaceutically acceptable salt or deuterated form thereof, wherein R⁰ is

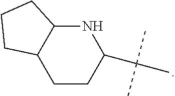

26. The compound of claim 11 or 12, or a pharmaceutically acceptable salt or deuterated form thereof, wherein R⁰ is 7-12 membered bridged heterocycle containing 1-3 heteroatoms selected from N, S, O, wherein the bridged heterocycle is optionally substituted with 1-3 R⁸.

27. The compound of claim 26, or a pharmaceutically acceptable salt or deuterated form thereof, wherein R⁰ is 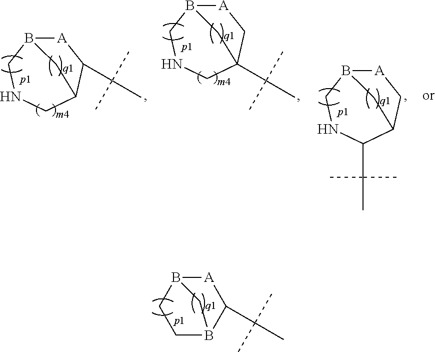
each of which is optionally substituted with 1-4 R⁸, wherein
A is a bond, —O—, —O—CH₂—, —CH₂—O—CH₂—, —CH₂OCH₂CH₂—, —CH₂—, —CH₂CH₂—, or —CH₂NH—B is N or CH,
m⁴ is 0 or 1,
p¹ is 0, 1, or 2,
q¹ is 1, 2, or 3.

28. The compound of claim 26 or 27, or a pharmaceutically acceptable salt or deuterated form thereof, wherein R⁰ is

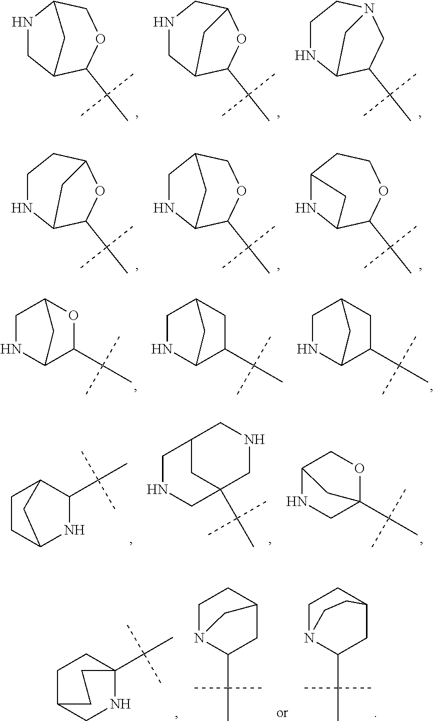

29. The compound of claim 1, or a pharmaceutically acceptable salt or deuterated form thereof, wherein R⁰ is

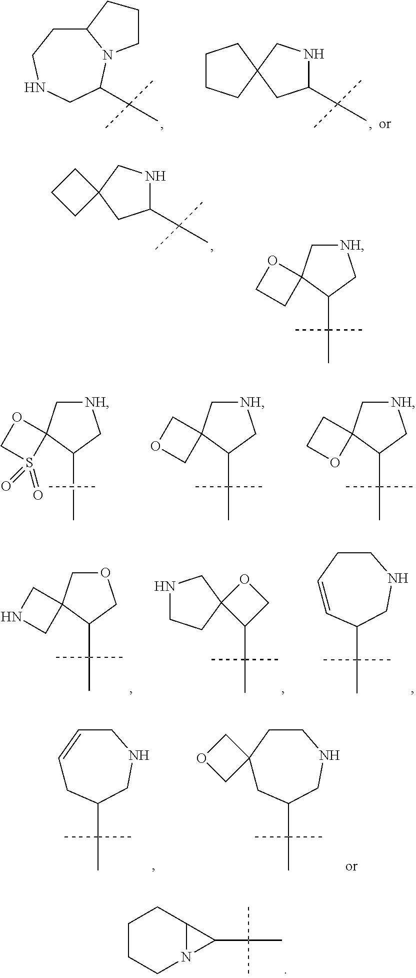

30. The compound of claim 1, or a pharmaceutically acceptable salt or deuterated form thereof, wherein R¹ is 5-12 membered carbocyclyl optionally substituted with 1-3 R^{g} group.

31. The compound of claim 30, or a pharmaceutically acceptable salt or deuterated form thereof, wherein R¹ is

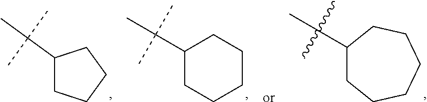

32. The compound of claim 1, or a pharmaceutically acceptable salt or deuterated form thereof, wherein R¹ is 6-18 membered carbocyclyl optionally substituted with 1-3 R^{g}.

33. The compound of claim 32, or a pharmaceutically acceptable salt or deuterated form thereof, wherein R¹ is 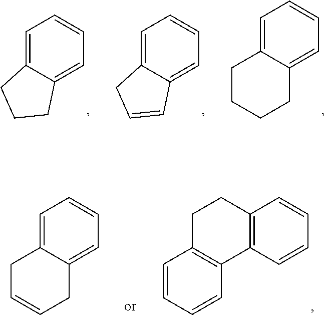
wherein L is attached to R¹ by replacing any hydrogen atom of R¹, and wherein each R¹ is optionally substituted with 1-3 R^{g}.

34. The compound of claim 32, or a pharmaceutically acceptable salt or deuterated form thereof, wherein R¹ is

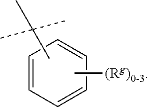

35. The compound of claim 34, or a pharmaceutically acceptable salt or deuterated form thereof, wherein R¹ is

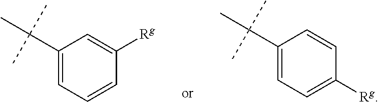

36. The compound of claim 34 or 35, or a pharmaceutically acceptable salt or deuterated form thereof, wherein R¹ is

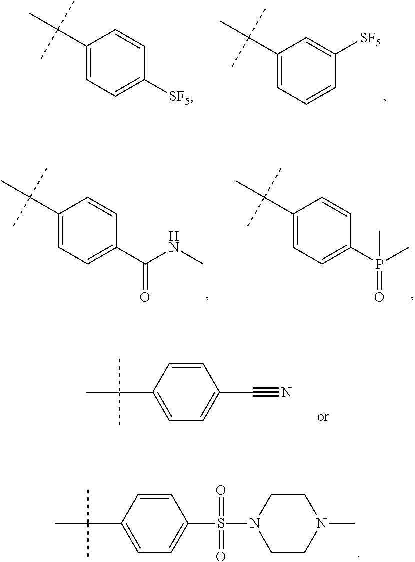

37. The compound of claim 34, or a pharmaceutically acceptable salt or deuterated form thereof, wherein R¹ is

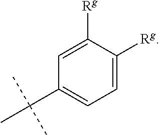

38. The compound of claim 37, or a pharmaceutically acceptable salt or deuterated form thereof, wherein R¹ is

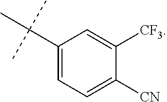

39. The compound of claim 1, or a pharmaceutically acceptable salt or deuterated form thereof, wherein R¹ is 5-12 membered monocyclic heterocyclyl containing heteroatoms selected from N, S or O, wherein the monocyclic heterocyclyl is optionally substituted with 1-3 R^{g} group.

40. The compound of claim 39, or a pharmaceutically acceptable salt or deuterated form thereof, wherein R¹ is 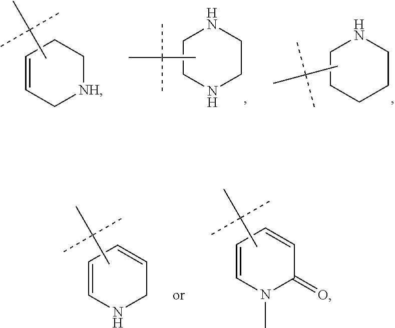
wherein each one of R¹ is optionally substituted with 1-3 R^{g}.

41. The compound of claim 1, or a pharmaceutically acceptable salt or deuterated form thereof, wherein R¹ is 5-12 membered monocyclic heteroaryl containing heteroatoms selected from N, S or O, wherein each one of the monocyclic heteroaryl is optionally substituted with 1-3 R^{g} group.

42. The compound of claim 41, or a pharmaceutically acceptable salt or deuterated form thereof, wherein R¹ is

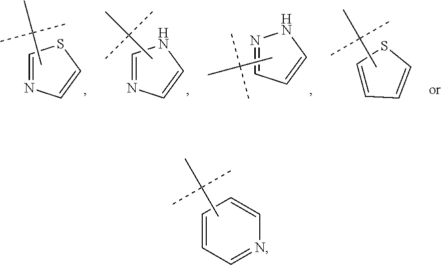

43. The compound of claim 41 or 42, or a pharmaceutically acceptable salt or deuterated form form thereof, wherein each R^{g} is independently H, halogen, C₁-C₆ alkyl, OSO₂C₁₋₆alkyl, or CN.

44. The compound of claim 41 or 42, or a pharmaceutically acceptable salt or deuterated form thereof, wherein R¹ is

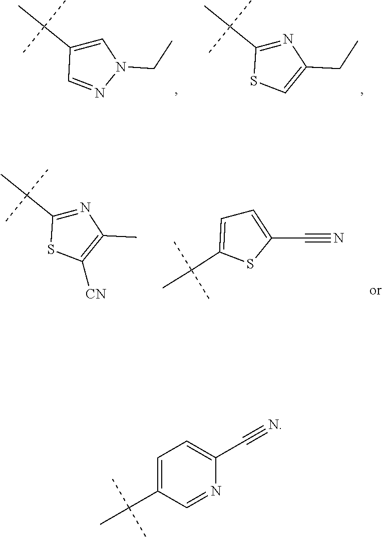

45. The compound of claim 1, or a pharmaceutically acceptable salt or deuterated form thereof, wherein R¹ is 7-14 membered bicyclic heteroaryl containing heteroatoms selected from N, S or O, wherein the bicyclic heteroaryl is optionally substituted with 1-4 R^{g}.

46. The compound of claim 1 or 45, or a pharmaceutically acceptable salt or deuterated form thereof, wherein R^{g} is R⁶ or R⁷, wherein
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH.

47. The compound of claim 45, or a pharmaceutically acceptable salt or deuterated form thereof, wherein R¹ is 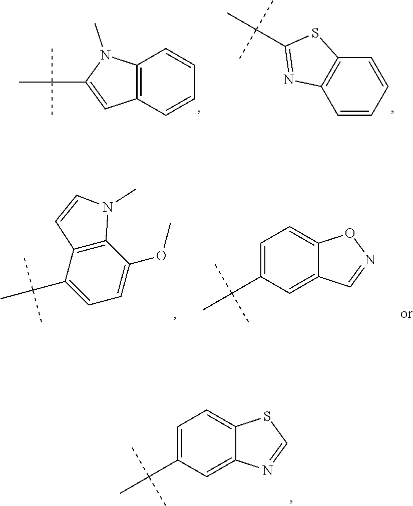
each of which is optionally substituted with 1-3 R^{g} group.

48. The compound of claim 45 or 46, or a pharmaceutically acceptable salt or deuterated form thereof, wherein R¹ is 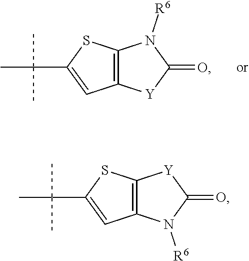
wherein
Y is independently O, S, CHR⁶ or NR⁶; and
R⁶ is C₁₋₆alkyl optionally substituted by 1, 2 or 3 F, or by OH, O—C₁₋₆alkyl, N(C₁₋₆alkyl)₂, cycloalkyl, or heterocyclyl.

49. The compound of claim 48, or a pharmaceutically acceptable salt or deuterated form thereof, wherein R¹ is

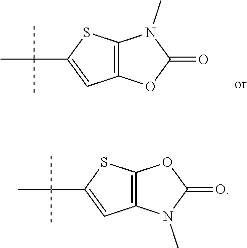

50. The compound of claim 45 or 46, or a pharmaceutically acceptable salt or deuterated form thereof, wherein R¹ is 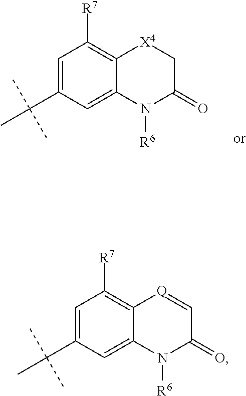
X⁴ is NR⁶, O, CR⁷, CR¹⁴R¹⁵, S, S(O) or S(O)₂,
Q is CH or N,
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH;
R¹⁴ and R¹¹ are each independently selected from H, halogen, C₁₋₆ alkyl, C₁₋₆alkoxy, C₂₋₆ alkenyl, C₂₋₆ alkynyl, C₂₋₆alkenyloxy, C₂₋₆ alkynyloxy, C₃₋₆ cycloalkyl, C₃₋₆ cycloalkoxy, cyano, hydroxyl, NH₂, COOH, —S(O)C₁₋₆alkyl, —S(O)₂C₁₋₆ alkyl, —S(O)₂C₃₋₆cycloalkyl, —SO₂-3-7 membered heterocyclyl, and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups selected from halogen, cyano, hydroxyl, NH₂ and —COOH;
alternatively, R¹⁴ and R¹⁵ form ═O.

51. The compound of any one of claims 45-46, or a pharmaceutically acceptable salt or deuterated form thereof, wherein R¹ is 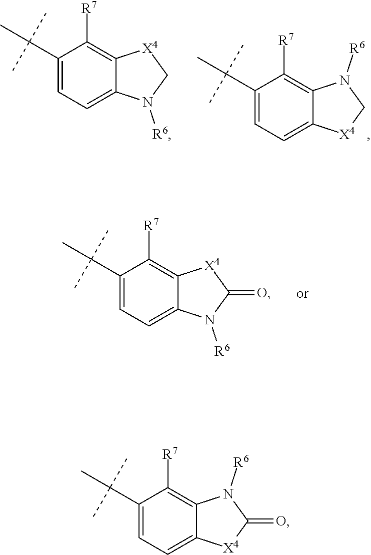
each X⁴ is independently NR, O, CR¹⁴R¹⁵, S, S(O) or S(O)₂,
each R⁶ is independently selected from H, C₁₋₆ alkyl, —COC₁₋₆ alkyl, C₂₋₆ alkenyl, C₂₋₆alkynyl and C₃₋₆cycloalkyl; wherein the alkyl, alkenyl, alkynyl and cycloalkyl is optionally substituted with 1-3 groups selected from halogen, cyano, hydroxyl, NH₂ and COOH;
each R⁷ is independently selected from H, halogen, C₁₋₆ alkyl, C₁₋₆ alkoxy, C₂₋₆ alkenyl, C₂₋₆ alkynyl, C₃₋₆cycloalkyl, cyano, hydroxyl, NH₂, NHC₁₋₆ alkyl, N(C₁₋₆ alkyl)₂, COOH, COC₁₋₆ alkyl, COOC₁₋₆ alkyl, CONHC₁₋₆ alkyl, CON(C₁₋₆ alkyl)₂, NHCOC₁₋₆ alkyl, and a 4-7 membered heterocycle containing 1-3 heteroatoms selected from N, S or O; wherein the alkyl, alkenyl, alkynyl, cycloalkyl and heterocycle groups within R⁷ are optionally substituted with 1-3 groups selected from halogen, cyano, hydroxyl, NH₂ and COOH;
R¹⁴ and R¹¹ are each independently selected from H, halogen, C₁₋₆ alkyl, C₁₋₆alkoxy, C₂₋₆ alkenyl, C₂₋₆ alkynyl, C₂₋₆alkenyloxy, C₂₋₆ alkynyloxy, C₃₋₆ cycloalkyl, C₃₋₆ cycloalkoxy, cyano, hydroxyl, NH₂, COOH, —S(O)C₁₋₆alkyl, —S(O)₂C₁₋₆ alkyl, —S(O)₂C₃₋₆cycloalkyl, —SO₂-3-7 membered heterocyclyl, and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups selected from halogen, cyano, hydroxyl, NH₂ and —COOH;
alternatively, R¹⁴ and R¹⁵ form ═O.

52. The compound of claim 50, or a pharmaceutically acceptable salt or deuterated form thereof, wherein
each X⁴ is independently 0, S, NR⁶ or CR¹⁴R¹⁵;
R⁶ is C₁₋₆alkyl optionally substituted by 1, 2 or 3 F, or by OH, O—C₁₋₆alkyl, N(C₁₋₆alkyl)₂, cycloalkyl, or heterocyclyl;
R⁷ is H, halo or C₁₋₆alkyl, R¹⁴ and R¹⁵ are independently H, halo or C₁₋₆alkyl.

53. The compound of claim 52 or 53, or a pharmaceutically acceptable salt or deuterated form thereof, wherein each X⁴ is independently NH, O, S, CHF, or CHF₂.

54. The compound of any one of claims 52-53, or a pharmaceutically acceptable salt or deuterated form thereof, wherein R is

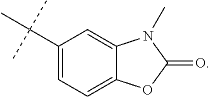

55. The compound of any one of claims 52-53, or a pharmaceutically acceptable salt or deuterated form thereof, wherein R¹ is

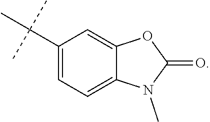

56. The compound of any one of claims 45-46 or 51, or a pharmaceutically acceptable salt or deuterated form thereof, wherein R¹ is

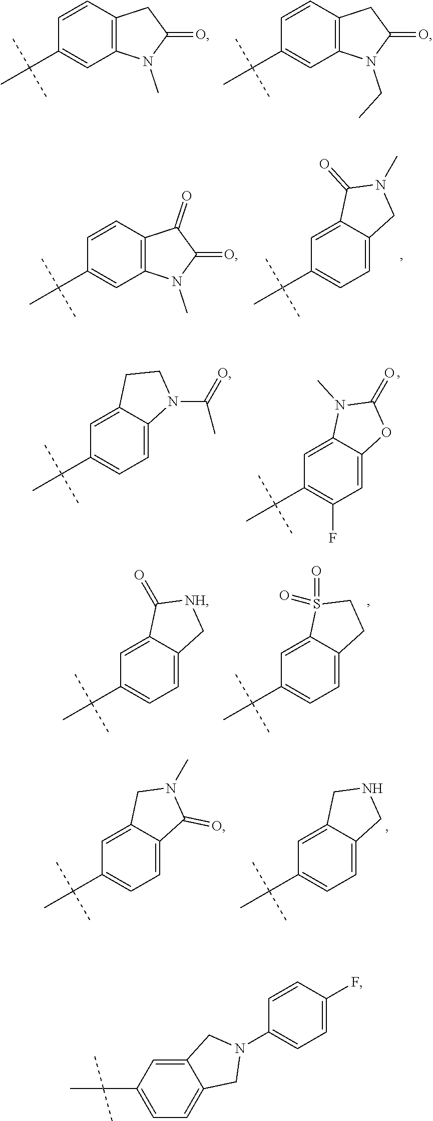

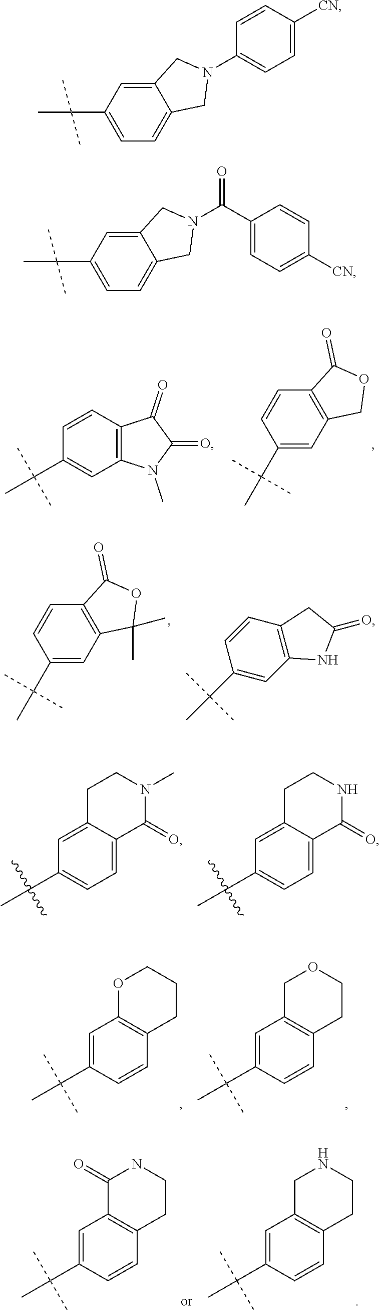

57. The compound of any one of claims 45-46, 51 or 56 or a pharmaceutically acceptable salt or deuterated form thereof, wherein R¹ is

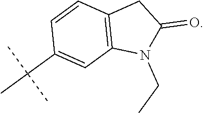

58. The compound of claim 45 or 46, or a pharmaceutically acceptable salt or deuterated form thereof, wherein R¹ is 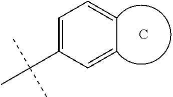
optionally substituted with 1-4 R⁶ or R⁷ group, wherein ring C is a 7-8 membered heterocycle comprising 1-3 heteroatoms selected from O, S and N.

59. The compound of claim 58, or a pharmaceutically acceptable salt or deuterated form thereof, wherein R¹ is 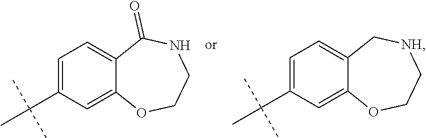
each of which is optionally substituted with 1-4 R⁶ or R⁷.

60. The compound of claim 58 or 59, or a pharmaceutically acceptable salt or deuterated form thereof, wherein R¹ is

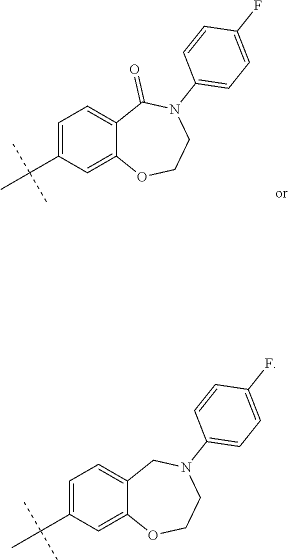

61. The compound of claim 1, or a pharmaceutically acceptable salt or deuterated form thereof, wherein R¹ is 7-20 membered tricyclic heteroaryl containing heteroatoms selected from N, S or O, wherein the tricyclic heteroaryl is optionally substituted with 1-5 R^{g}, wherein R^{g} is R⁶, R⁷, R¹¹, R¹⁴ or R¹⁵.

62. The compound of claim 1 or 61, or a pharmaceutically acceptable salt or deuterated form thereof, wherein R¹ is 7-20 membered spiro tricyclic heteroaryl containing heteroatoms selected from N, S or O, wherein the tricyclic heteroaryl is optionally substituted with 1-5 R^{g}.

63. The compound of any one of claims 61-62, or a pharmaceutically acceptable salt or deuterated form thereof, wherein R¹ is 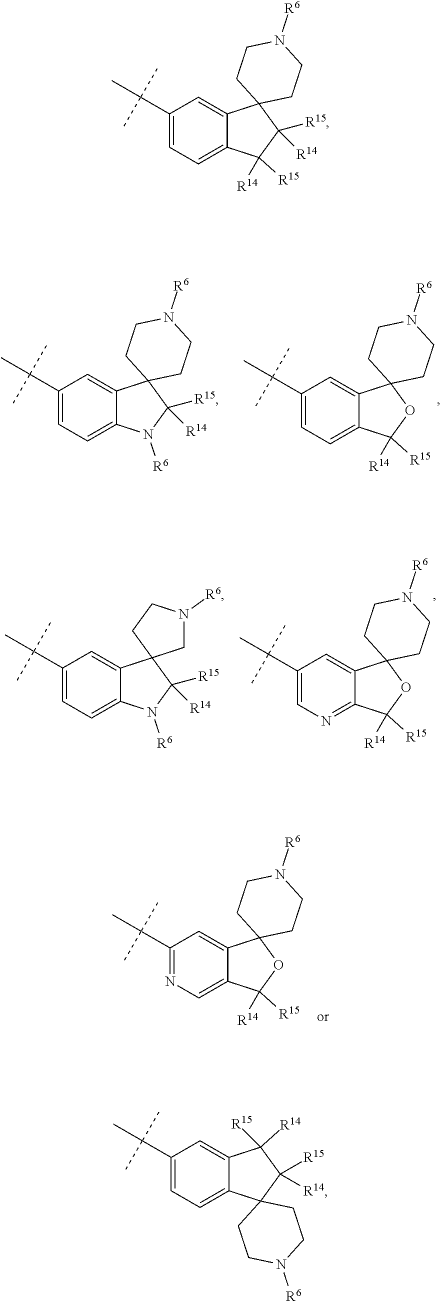
wherein
each R⁶ is independently selected from H, C₁₋₆ alkyl, —COC₁₋₆ alkyl, C₂₋₆ alkenyl, C₂₋₆alkynyl, C₃₋₆cycloalkyl or alkylene-O-alkyl; wherein the alkyl, alkenyl, alkynyl and cycloalkyl is optionally substituted with 1-3 groups selected from halogen, cyano, hydroxyl, NH₂ and COOH; and,
R¹⁴ and R¹⁵ are each independently selected from H, halogen, C₁₋₆ alkyl, C₁₋₆alkoxy, C₂₋₆ alkenyl, C₂₋₆ alkynyl, C₂₋₆alkenyloxy, C₂₋₆ alkynyloxy, C₃₋₆ cycloalkyl, C₃₋₆ cycloalkoxy, cyano, hydroxyl, NH₂, COOH, —S(O)C₁₋₆alkyl, —S(O)₂C₁₋₆ alkyl, —S(O)₂C₃₋₆cycloalkyl, —SO₂-3-7 membered heterocyclyl and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups selected from halogen, cyano, hydroxyl, NH₂ and —COOH;
alternatively, R¹⁴ and R¹⁵ form ═O.

64. The compound of claim 62-63, or a pharmaceutically acceptable salt or deuterated form thereof, wherein R¹ is 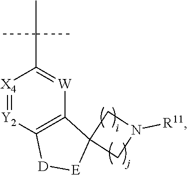
wherein
W, X₄ and Y₂ are each independently CH or N, provided that a maximum of one of W, X₄ and Y₂ can be N;
D-E is N(H)—C(═O), N(C₁₋₆alkyl)-C(═O), CH₂CH₂, C(═O)—O or CH₂—O;
R¹¹ is H, C₁₋₆alkyl, alkylene-O-alkyl, or heterocyclyl; and
i and j are each independently 1, 2 or 3; provided that the sum of i+j is 2, 3 or 4.

65. The compound of any one of claims 61-64, or a pharmaceutically acceptable salt or deuterated form thereof, wherein R¹ is

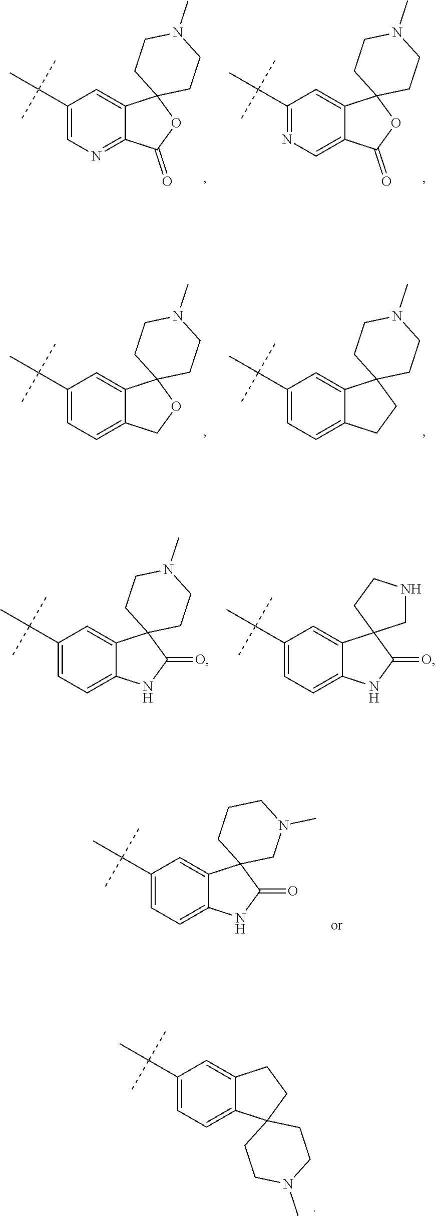

66. The compound of any one of claims 61-65, or a pharmaceutically acceptable salt or deuterated form thereof, wherein R is

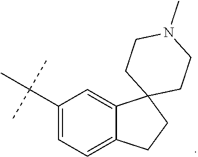

67. The compound of claim 1, or a pharmaceutically acceptable salt or deuterated form thereof, wherein R¹ is 7-20 membered fused tricyclic heteroaryl containing heteroatoms selected from N, S or O, wherein the tricyclic heteroaryl is optionally substituted with 1-5 R^{g}.

68. The compound of claim 1 or 67, or a pharmaceutically acceptable salt or deuterated form thereof, wherein R¹ is 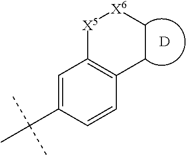
optionally substituted with 1-5 R^{g}, wherein
X⁵ and X⁶ are each independently selected from single bond, —C(R¹⁴R¹⁵)—O—, —C(R¹⁴R¹⁵)—C(R¹⁴R¹⁵)—, —OC(R¹⁴R¹⁵)—, —C(R¹⁴R¹⁵)—, —O—, and —NR⁶—;
R¹⁴ and R¹⁵ are each independently selected from H, halogen, C₁₋₆ alkyl, C₁₋₆alkoxy, C₂₋₆ alkenyl, C₂₋₆ alkynyl, C₂₋₆alkenyloxy, C₂₋₆ alkynyloxy, C₃₋₆ cycloalkyl, C₃₋₆ cycloalkoxy, cyano, hydroxyl, NH₂, COOH, —S(O)C₁₋₆alkyl, —S(O)₂C₁₋₆ alkyl, —S(O)₂C₃₋₆cycloalkyl, —SO₂-3-7 membered heterocyclyl and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups selected from halogen, cyano, hydroxyl, NH₂ and —COOH;
each R⁶ is independently selected from H, C₁₋₆ alkyl, —COC₁₋₆ alkyl, C₂₋₆ alkenyl, C₂₋₆alkynyl, C₃₋₆cycloalkyl or alkylene-O-alkyl; wherein the alkyl, alkenyl, alkynyl and cycloalkyl is optionally substituted with 1-3 groups selected from halogen, cyano, hydroxyl, NH₂ and COOH;
Ring D is selected from aryl and 5-8 membered heteroaryl containing 1-3 heteroatoms, wherein aryl and said 5-8 membered heteroaryl containing 1-3 heteroatoms each independently optionally substituted with 1-3 R^{g},
provided when ring D is aryl, X⁵ and X⁶ are not both —C(R¹⁴R¹⁵)—C(R¹⁴R¹⁵)— or —C(R¹⁴R¹⁵)—.

69. The compound of claim 68, or a pharmaceutically acceptable salt or deuterated form thereof, wherein R¹ is 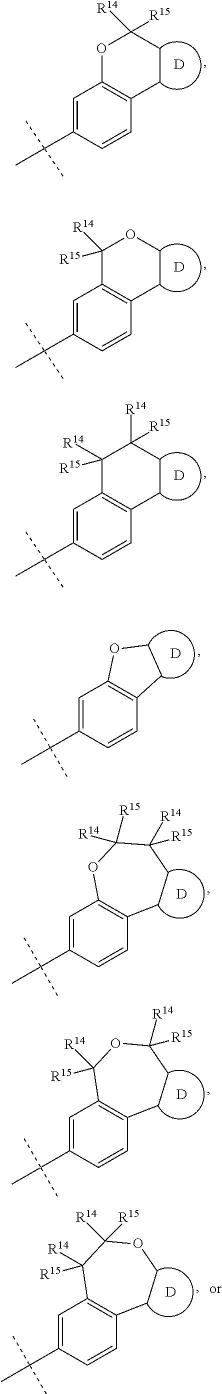 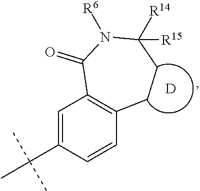
each of which is optionally substituted with 1-5 R^{g}.

70. The compound of claim 68 or 69, or a pharmaceutically acceptable salt or deuterated form thereof, wherein R¹ is 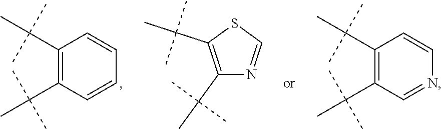
each of which is optionally substituted with 1-3 R^{g}.

71. The compound of claim 68 or 70, or a pharmaceutically acceptable salt or deuterated form thereof, wherein R¹ is

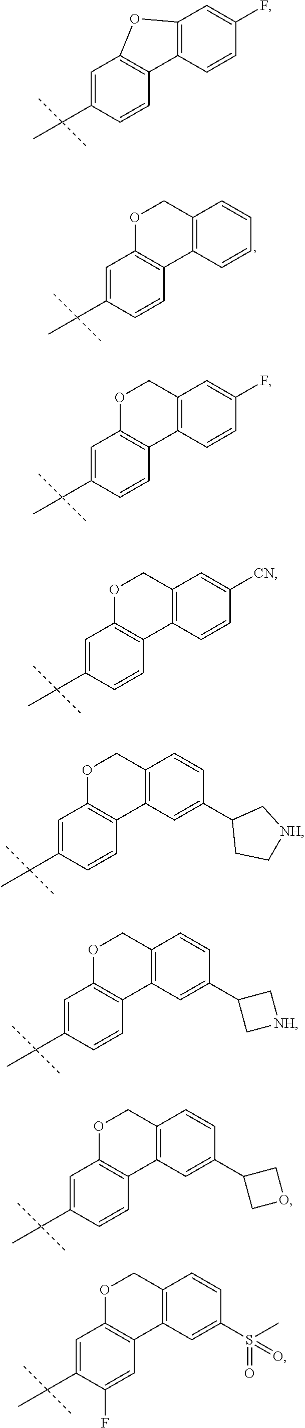

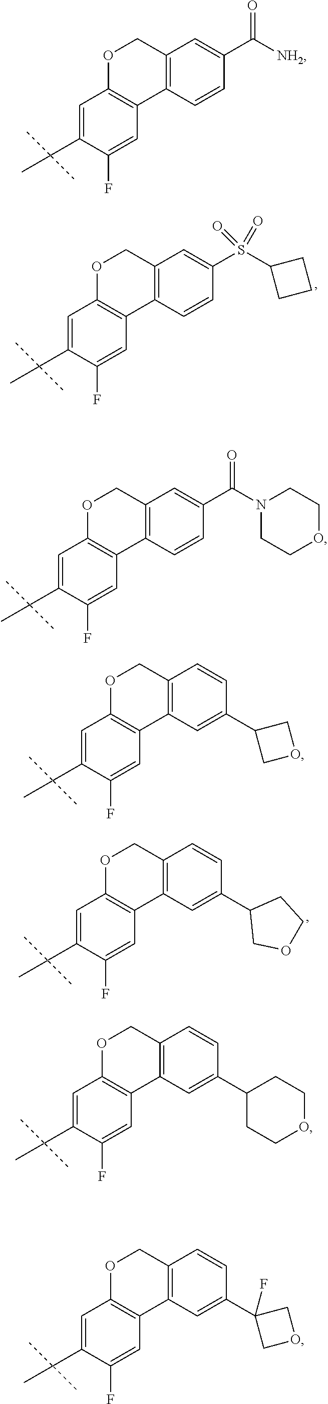

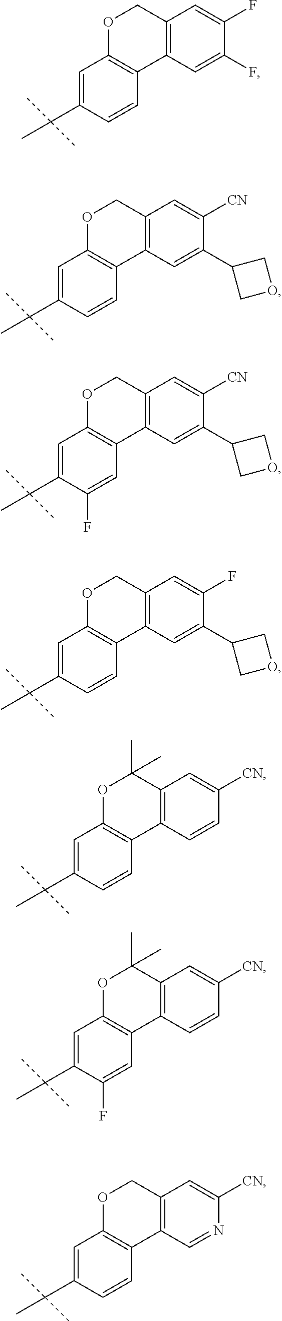

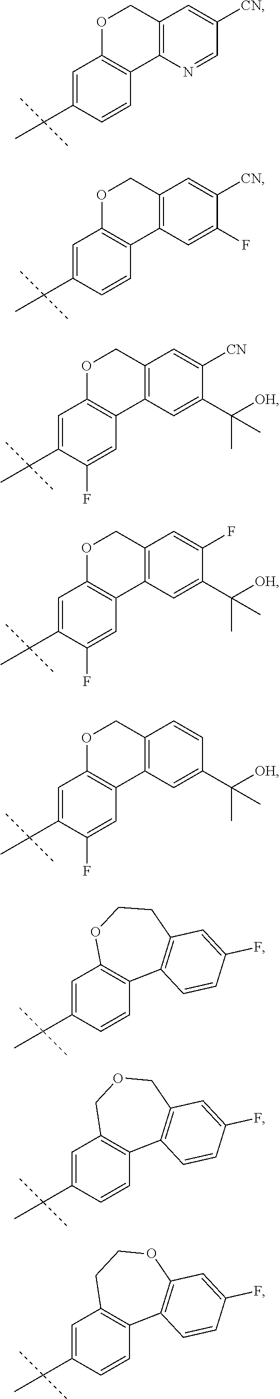

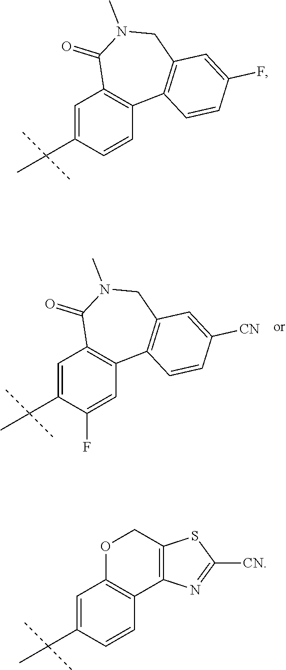

72. The compound of any one of claims 1-71, or a pharmaceutically acceptable salt or deuterated form thereof, wherein L is a polycyclic cycloalkylene.

73. The compound of any one of claims 1-72, or a pharmaceutically acceptable salt or deuterated form thereof, wherein the polycyclic cycloalkylene comprises 4-10 carbon atoms.

74. The compound of any one of claims 1-73, or a pharmaceutically acceptable salt or deuterated form thereof, wherein the polycyclic cycloalkylene is connected to R¹ at a first quaternary carbon of the polycyclic cycloalkylene and independently connected to 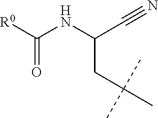
through a second quaternary carbon of the polycyclic cycloalkylene.

75. The compound of any one of claims 1-74, or a pharmaceutically acceptable salt or deuterated form thereof, wherein the polycyclic cycloalkylene is

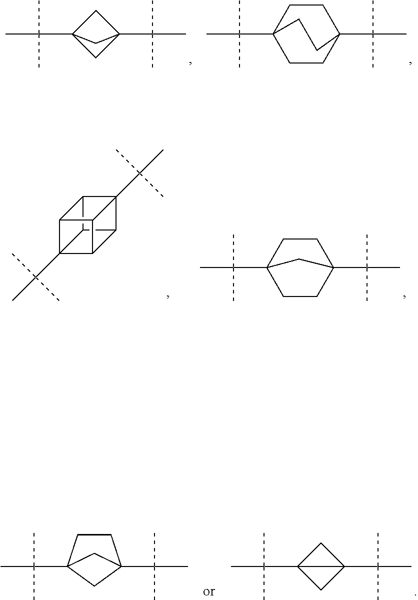

76. The compound of any one of claims 1-75, or a pharmaceutically acceptable salt or deuterated form thereof, wherein the polycyclic cycloalkylene is

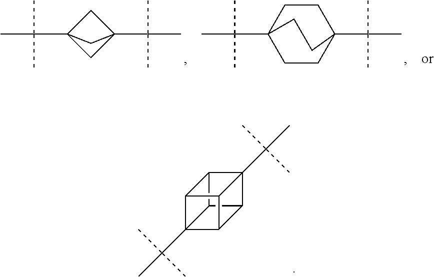

77. The compound of any one of claims 1-76, or a pharmaceutically acceptable salt or deuterated form thereof, wherein L is a polycyclic heteroarylene substituted by 0-4 R¹⁰.

78. The compound of claim 77, or a pharmaceutically acceptable salt or deuterated form thereof, wherein the polycyclic heteroarylene comprises 8-12 ring atoms and is substituted by 0-4 R¹⁰.

79. The compound of any one of claims 77-78, or a pharmaceutically acceptable salt or deuterated form thereof, wherein the polycyclic heteroarylene comprises 8-10 ring atoms and is substituted by 0-4 R¹⁰.

80. The compound of any one of claims 77-79, or a pharmaceutically acceptable salt or deuterated form thereof, wherein the polycyclic heteroarylene comprises 8-9 ring atoms and is substituted by 0-4 R¹⁰.

81. The compound of claim 77, or a pharmaceutically acceptable salt or deuterated form thereof, wherein the polycyclic heteroarylene is 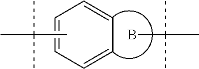
substituted by 0-4 R¹⁰, wherein ring B is a heteroaryl ring.

82. The compound of any one of claims 77-81, or a pharmaceutically acceptable salt or deuterated form thereof, wherein the polycyclic heteroarylene comprises 9 ring atoms and 1 or 2 heteroatoms selected from O, N, or S, and the polycyclic heteroarylene is substituted by 0-4 R¹⁰.

83. The compound of any one of claims 77-82, or a pharmaceutically acceptable salt or deuterated form thereof, wherein the polycyclic heteroarylene is 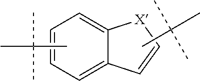
substituted by 0-4 R¹⁰, wherein X′ is O, S, NH or N(C₁₋₆ alkyl).

84. The compound of any one of claims 77-83, or a pharmaceutically acceptable salt or deuterated form thereof, wherein the polycyclic heteroarylene is benzothienylene, indolylene or benzofuranylene substituted by 0-4 R¹⁰.

85. The compound of any one of claims 77-84, or a pharmaceutically acceptable salt or deuterated form thereof, wherein the polycyclic heteroarylene is 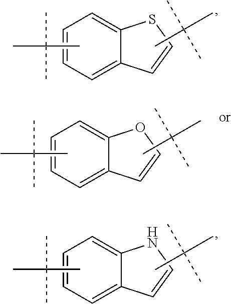
each of which is substituted by 0-4 R¹⁰.

86. The compound of claim 85, or a pharmaceutically acceptable salt or deuterated form thereof, wherein 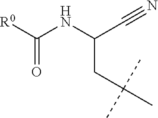
is attached to the phenyl ring of 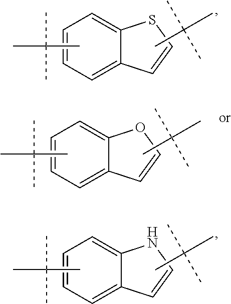
and R¹ is attached the 5-membered ring of 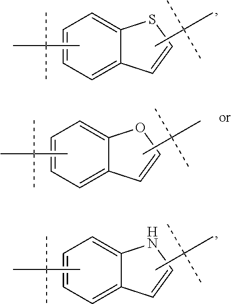
wherein 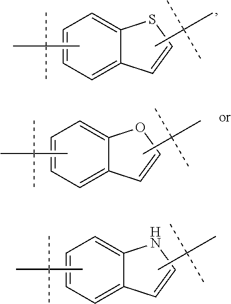
is substituted by 0-4 R¹⁰.

87. The compound of claim 85, or a pharmaceutically acceptable salt or deuterated form thereof, wherein 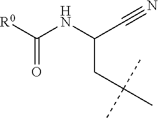
is attached to the 5-membered ring of 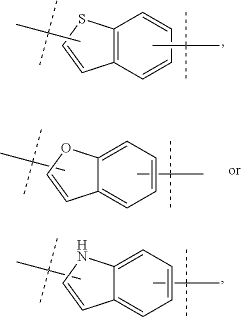
and R¹ is attached to the phenyl ring of 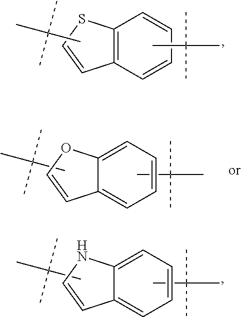
and wherein 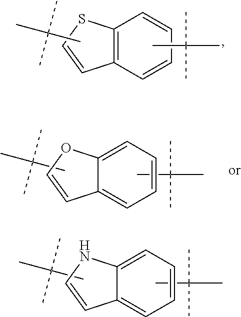
is substituted by 0-4 R¹⁰.

88. The compound of any one of claims 77-87, or a pharmaceutically acceptable salt or deuterated form thereof, wherein the polycyclic heteroarylene is

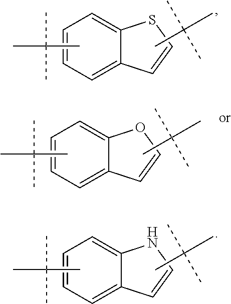

89. The compound of any one of claims 77-88, or a pharmaceutically acceptable salt or deuterated form thereof, wherein the polycyclic heteroarylene is

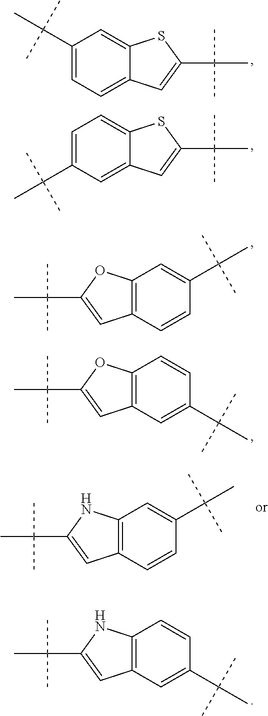

90. The compound of any one of claims 77-87, or a pharmaceutically acceptable salt or deuterated form thereof, wherein L is

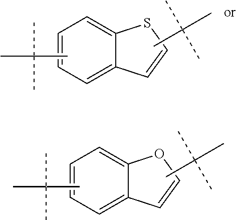

91. The compound of any one of claim 90, or a pharmaceutically acceptable salt or deuterated form thereof, wherein L is

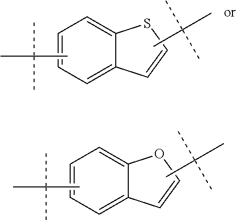

92. The compound of any one of claim 90 or 91, or a pharmaceutically acceptable salt or deuterated form thereof, wherein R¹⁰ is halo.

93. The compound of any one of claim 90-92, or a pharmaceutically acceptable salt or deuterated form thereof, wherein L is

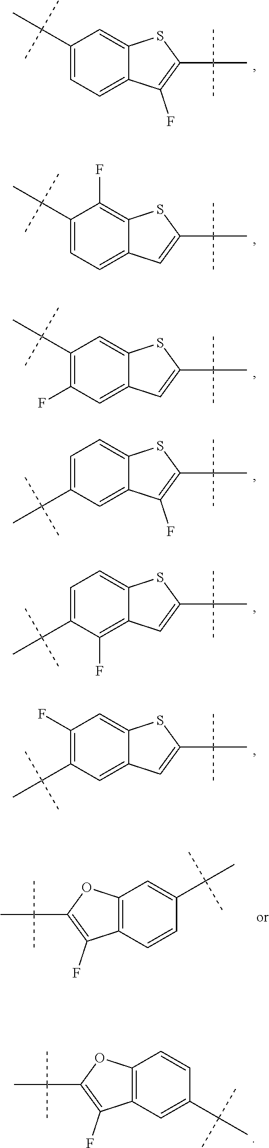

94. The compound of any one of claims 90-93, or a pharmaceutically acceptable salt or deuterated form thereof, wherein L is

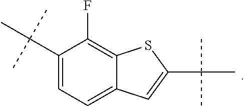

95. The compound of any one of claims 77-80, or a pharmaceutically acceptable salt or deuterated form thereof, wherein the polycyclic heteroarylene comprises 8 ring atoms and 1 or 2 heteroatoms selected from O, N, or S, and the polycyclic heteroarylene is substituted by 0-4 R¹⁰.

96. The compound of any one of claims 77-80 or 95, or a pharmaceutically acceptable salt or deuterated form thereof, wherein the polycyclic heteroarylene is 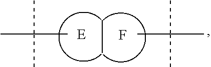
wherein each of ring E and ring F is 5-membered heteroaryl or 5-membered heterocyclyl comprising 1-3 heteroatoms selected from O, N or S.

97. The compound of any one of claims 77-80 or 95-96, or a pharmaceutically acceptable salt or deuterated form thereof, wherein the polycyclic heteroarylene is 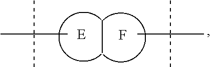
wherein each of ring E and ring F is 5-membered heteroaryl comprising 1-3 heteroatoms selected from O, N or S.

98. The compound of any one of claims 77-80 or 95-96, or a pharmaceutically acceptable salt or deuterated form thereof, wherein the polycyclic heteroarylene is

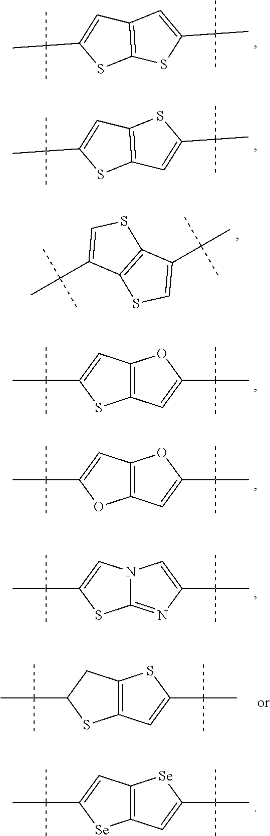

99. The compound of any one of claims 77-80, 95-96 or 98, or a pharmaceutically acceptable salt or deuterated form thereof, wherein the polycyclic heteroarylene is

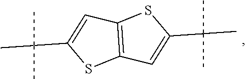

100. The compound of any one of claim 1-71, or a pharmaceutically acceptable salt or deuterated form thereof, wherein L is polycyclic arylene.

101. The compound of claim 100, or a pharmaceutically acceptable salt or deuterated form thereof, wherein L is

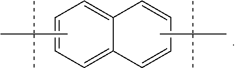

102. The compound of claim 100 or 101, or a pharmaceutically acceptable salt or deuterated form thereof, wherein L is

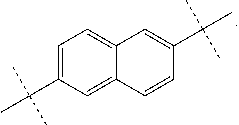

103. The compound of any one of claims 1-71, wherein the compound is of Formula (II), 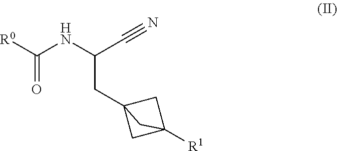
or a pharmaceutically acceptable salt or deuterated form thereof.

104. The compound of any one of claims 1-71, wherein the compound is of Formula (III), 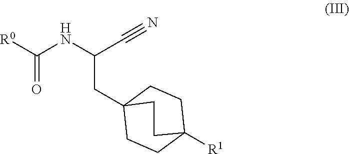
or a pharmaceutically acceptable salt or deuterated form thereof.

105. The compound of any one of claims 1-71, wherein the compound is of Formula (IV), 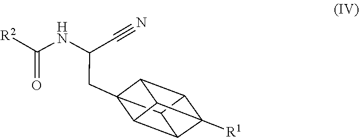
or a pharmaceutically acceptable salt or deuterated form thereof.

106. The compound of claim 1, wherein the compound is of Formula (V), 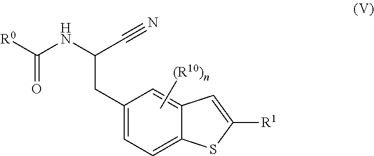
or a pharmaceutically acceptable salt or deuterated form thereof,
wherein n is 0 or 1, and R¹⁰ is substituted on the phenyl ring or the thiophenyl ring of the benzothienylene ring.

107. The compound of claim 1, wherein the compound is of Formula (VI), 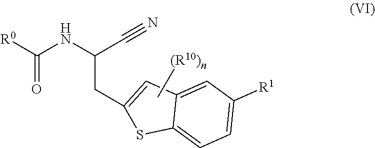
or a pharmaceutically acceptable salt or deuterated form thereof,
wherein n is 0 or 1, and R¹⁰ is substituted on the phenyl ring or the thiophenyl ring of the benzothienylene ring.

108. The compound of claim 1, wherein the compound is of Formula (VII), 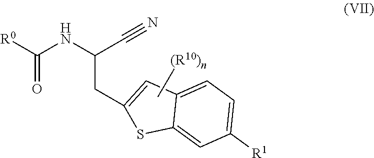
or a pharmaceutically acceptable salt or deuterated form thereof,
wherein n is 0 or 1, and R¹⁰ can be substituted on the phenyl ring or the thiophenyl ring of the benzothienylene ring.

109. The compound of any one of claims 103-108, or a pharmaceutically acceptable salt or deuterated form thereof, wherein n is 0.

110. The compound of any one of claims 103-108, or a pharmaceutically acceptable salt or deuterated form thereof, wherein n is 1 and R¹⁰ is halo.

111. The compound of any one of claims 103-108 or 110, or a pharmaceutically acceptable salt or deuterated form thereof, wherein n is 1 and R¹⁰ is F.

112. The compound of any one of claims 1-111, or a pharmaceutically acceptable salt or deuterated form thereof, wherein
R¹ is 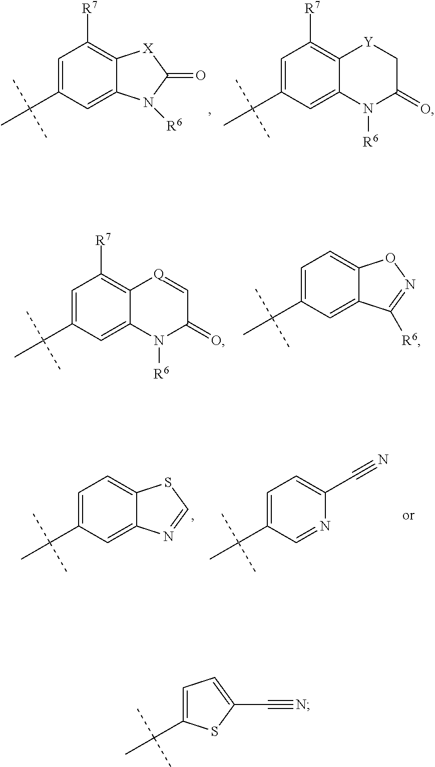
X is O, S or CF₂;
Y is O or S;
Q is CH or N;
R⁶ is C₁₋₃alkyl, wherein said C₁₋₃alkyl is optionally substituted by 1, 2 or 3 F, OH, OC₁₋₃alkyl, N(C₁₋₃alkyl)₂, cyclopropyl, or tetrahydropyran; and
R⁷ is H, F, C₁ or CH₃.

113. The compound of any one of claims 1-112, or a pharmaceutically acceptable salt or deuterated form thereof, wherein
R¹ is 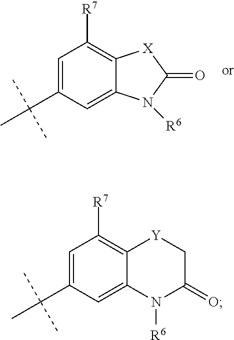
X is O, S or CF₂;
Y is O or S;
R⁶ is C₁₋₃alkyl, wherein said C₁₋₃alkyl is optionally substituted by 1, 2 or 3 F, OH, OC₁₋₃alkyl, N(C₁₋₃alkyl)₂, cyclopropyl, or tetrahydropyran; and
R⁷ is H, F, C₁ or CH₃.

114. The compound of any one of claims 1-112, or a pharmaceutically acceptable salt or deuterated form thereof, wherein R¹ is

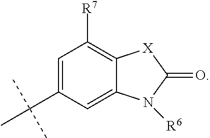

115. The compound of claim 114, or a pharmaceutically acceptable salt or deuterated form thereof, wherein, X is O; R⁶ is C₁₋₃alkyl; and R⁷ is H.

116. The compound of any one of claims 1-112, or a pharmaceutically acceptable salt or deuterated form thereof, wherein
R¹ is 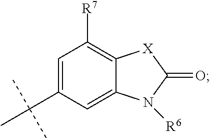
X is O;
R⁶ is C₁₋₃alkyl, wherein said C₁₋₃alkyl is optionally substituted by 1, 2 or 3 F; and
R⁷ is H.

117. The compound of any one of claims 1-112, or a pharmaceutically acceptable salt or deuterated form thereof, wherein,
R¹ is 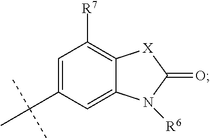
X is O;
R⁶ is C₁₋₃alkyl; and
R⁷ is H.

118. The compound of any one of claims 1-112, or a pharmaceutically acceptable salt thereof, wherein R¹ is

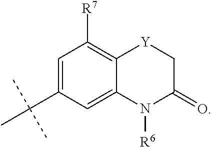

119. The compound of any one of claims 1-112, or a pharmaceutically acceptable salt or deuterated form thereof, wherein R¹ is

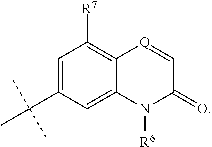

120. The compound of any one of claims 1-112, or a pharmaceutically acceptable salt or deuterated form thereof, wherein R¹ is

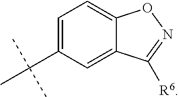

121. The compound of any one of claims 105-120, or a pharmaceutically acceptable salt or deuterated form thereof, wherein R⁶ is methyl.

122. The compound of any one of claims 105-120, or a pharmaceutically acceptable salt or deuterated form thereof, wherein R⁶ is ethyl.

123. The compound of any one of claims 105-120, or a pharmaceutically acceptable salt or deuterated form thereof, wherein R⁶ is propyl.

124. The compound of any one of claims 1-112, or a pharmaceutically acceptable salt or deuterated form thereof, wherein R¹ is

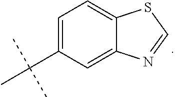

125. The compound of any one of claims 1-112, or a pharmaceutically acceptable salt or deuterated form thereof, wherein R¹ is

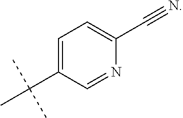

126. The compound of any one of claims 1-112, or a pharmaceutically acceptable salt or deuterated form thereof, wherein R¹ is

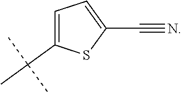

127. The compound of claim 1, or a pharmaceutically acceptable salt or deuterated form thereof, wherein the compound is of Formula (VIII): 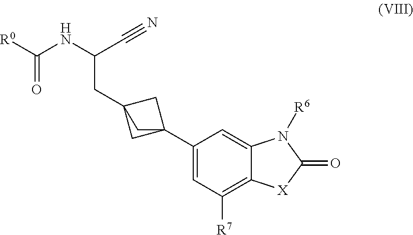
or a pharmaceutically acceptable salt or deuterated form thereof.

128. The compound of claim 1, or a pharmaceutically acceptable salt or deuterated form thereof, wherein the compound is of Formula (IX), 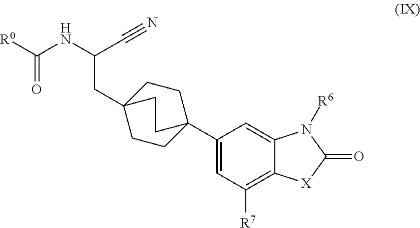
or a pharmaceutically acceptable salt or deuterated form thereof.

129. The compound of claim 1, or a pharmaceutically acceptable salt or deuterated form thereof, wherein the compound is of Formula (X), 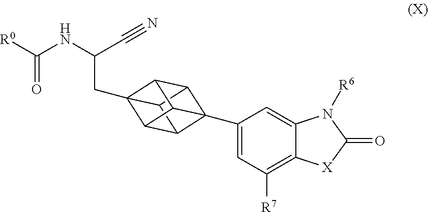
or a pharmaceutically acceptable salt or deuterated form thereof.

130. The compound of claim 1, or a pharmaceutically acceptable salt or deuterated form thereof, wherein the compound is of Formula (XI), 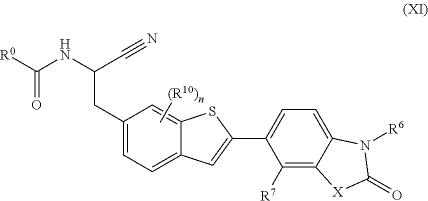
or a pharmaceutically acceptable salt or deuterated form thereof,
wherein
n is 0 or 1, and
R¹⁰ can be substituted on the phenyl ring or the thiophenyl ring of the benzothienylene ring.

131. The compound of claim 1, or a pharmaceutically acceptable salt or deuterated form thereof, wherein the compound is of Formula (XII), 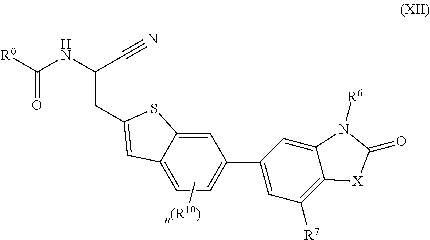
or a pharmaceutically acceptable salt or deuterated form thereof,
wherein
n is 0 or 1, and
R¹⁰ can be substituted on the phenyl ring or the thiophenyl ring of the benzothienylene ring.

132. The compound of claim 1, or a pharmaceutically acceptable salt or deuterated form thereof, wherein the compound is of Formula (XIII), 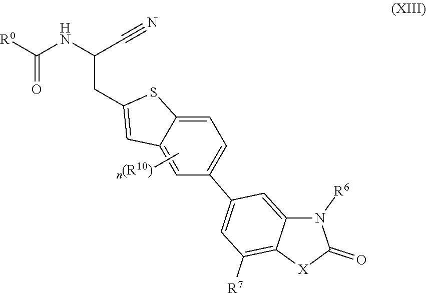
or a pharmaceutically acceptable salt or deuterated form thereof,
wherein
n is 0 or 1, and
R¹⁰ can be substituted on the phenyl ring or the thiophenyl ring of the benzothienylene ring.

133. The compound of claim 1, or a pharmaceutically acceptable salt or deuterated form thereof, wherein the compound is of Formula (XVI), 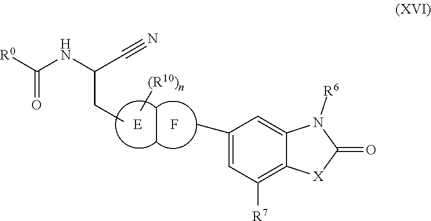
or a pharmaceutically acceptable salt or deuterated form thereof,
wherein
each of ring E and Ring F is 5-membered heteroarylene or 5-membered heterocyclylene,
n is 0 or 1, and
R¹⁰ is substituted on the ring E or ring F.

134. The compound of claim 133, wherein

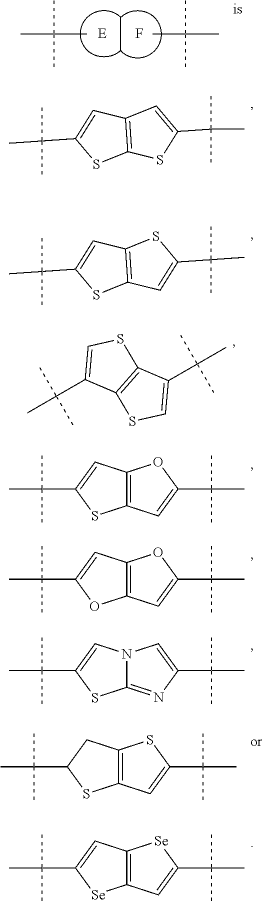

135. The compound of claim 133 or 134, wherein

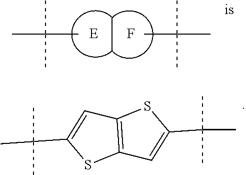

136. The compound of any one of claims 133-135, wherein R¹⁰ is halo.

137. The compound of claim 1, or a pharmaceutically acceptable salt or deuterated form thereof, wherein the compound is of Formula (XVII), 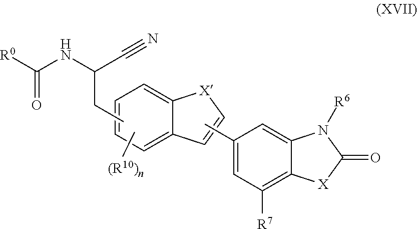
or a pharmaceutically acceptable salt or deuterated form thereof,
wherein
X′ is O, S, NH or N(C₁₋₆ alkyl),
n is 0 or 1, and
R¹⁰ is substituted on the phenyl ring or the 5-membered ring of

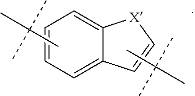

138. The compound of claim 1, or a pharmaceutically acceptable salt or deuterated form thereof, wherein the compound is of Formula (XVIII), 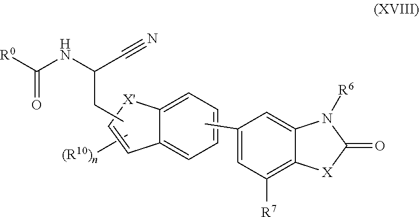
or a pharmaceutically acceptable salt or deuterated form thereof,
wherein
X′ is O, S, NH or N(C₁₋₆ alkyl),
n is 0 or 1, and
R¹⁰ is substituted on the phenyl ring or the 5-membered ring of

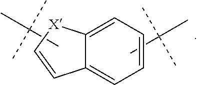

139. The compound of any one of claims 120-138, or a pharmaceutically acceptable salt or deuterated form thereof, wherein n is 0.

140. The compound of any one of claims 120-138, or a pharmaceutically acceptable salt or deuterated form thereof, wherein n is 1 and R¹⁰ is halo.

141. The compound of any one of claims 120-138 or 140, or a pharmaceutically acceptable salt or deuterated form thereof, wherein n is 1 and R¹⁰ is F.

142. The compound of any one of claims 127-141, or a pharmaceutically acceptable salt or deuterated form thereof, wherein X is O.

143. The compound of any one of claims 127-142, or a pharmaceutically acceptable salt or deuterated form thereof, wherein R⁶ is C₁₋₃ alkyl.

144. The compound of any one of claims 127-143, or a pharmaceutically acceptable salt or deuterated form thereof, wherein R⁷ is H.

145. The compound of any one of claims 127-144, or a pharmaceutically acceptable salt or deuterated form thereof, wherein X is O, R⁶ is C₁₋₃ alkyl and R⁷ is H.

146. The compound of any one of claims 1-145, or a pharmaceutically acceptable salt or deuterated form thereof, wherein R⁰ is

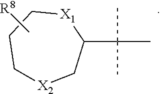

147. The compound of any one of claims 1-146, or a pharmaceutically acceptable salt or deuterated form thereof, wherein R⁰ is

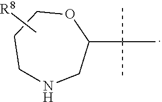

148. The compound of any one of claims 146-147, wherein R⁸ is H.

149. The compound of any one of claims 1-145, or a pharmaceutically acceptable salt or deuterated form thereof, wherein R⁰ is

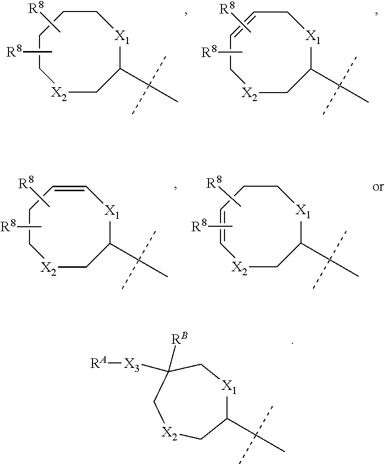

150. The compound of any one of claims 1-145 or 149, or a pharmaceutically acceptable salt or deuterated form thereof, wherein R⁰ is

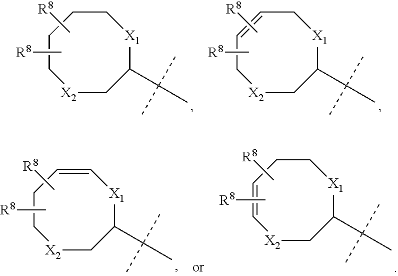

151. The compound of claim 150, or a pharmaceutically acceptable salt or deuterated form thereof, wherein R⁰ is

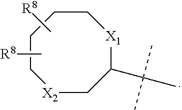

152. The compound of any one of claims 150-151, or a pharmaceutically acceptable salt or deuterated form thereof, wherein R⁰ is 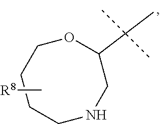
wherein each R⁸ is independently selected from H, halogen, oxo, cyano, hydroxyl, NH₂, NH(C₁₋₆alkyl), N(C₁₋₆alkyl)₂, COOH, C₁₋₆ alkyl, C₁₋₆ alkyl-OH, —CONH₂, —S(═O)NH₂, —S(O)₂NH₂, C₁₋₆alkoxy or halogenated C₁₋₆alkoxy.

153. The compound of any one of claims 150-152, or a pharmaceutically acceptable salt or deuterated form thereof, wherein R⁰ is 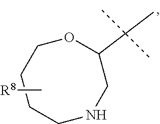
wherein each R⁸ is independently selected from H, halogen, C₁₋₆ alkyl, C₁₋₆alkoxy or halogenated C₁₋₆alkoxy.

154. The compound of claim 150, or a pharmaceutically acceptable salt or deuterated form thereof, wherein R⁰ is

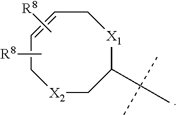

155. The compound of claim 150, or a pharmaceutically acceptable salt or deuterated form thereof, wherein R⁰ is

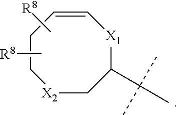

156. The compound of claim 150, or a pharmaceutically acceptable salt or deuterated form thereof, wherein R⁰ is

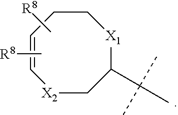

157. The compound of any one of claims 1 or 149-156, or a pharmaceutically acceptable salt or deuterated form thereof, wherein at least one R⁸ is H.

158. The compound of any one of claims 1 or 149-156, or a pharmaceutically acceptable salt or deuterated form thereof, wherein at least one R⁸ is methoxy.

159. The compound of any one of claims 1 or 149-156, or a pharmaceutically acceptable salt or deuterated form thereof, wherein at least one R⁸ is ethoxy.

160. The compound of any one of claims 1 or 149-156, or a pharmaceutically acceptable salt or deuterated form thereof, wherein at least one R⁸ is OH.

161. The compound of any one of claims 1-148, or a pharmaceutically acceptable salt or deuterated form thereof, wherein R⁰ is

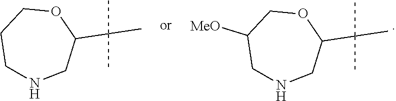

162. The compound of any one of claims 1-148 or 161, or a pharmaceutically acceptable salt deuterated form thereof, wherein R⁰ is

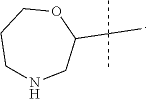

163. The compound of any one of claims 1-148 or 161, or a pharmaceutically acceptable salt or deuterated form thereof, wherein R⁰ is

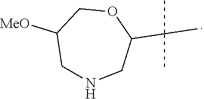

164. The compound of any one of claims 149-150, or a pharmaceutically acceptable salt or deuterated form thereof, wherein R⁰ is

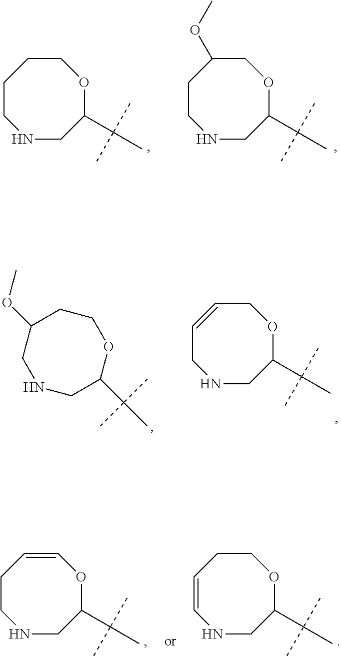

165. The compound of any one of claims 149-153 or 164, or a pharmaceutically acceptable salt or deuterated form thereof, wherein R⁰ is

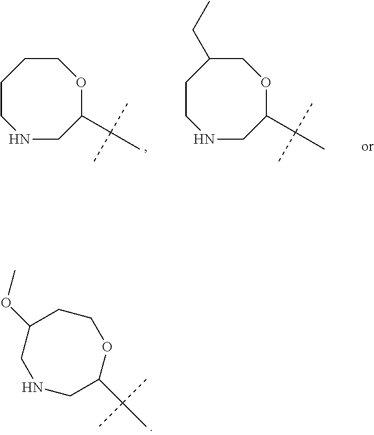

166. The compound of any one of claims 149-153 or 164-165, or a pharmaceutically acceptable salt or deuterated form thereof, wherein R⁰ is

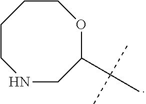

167. The compound of any one of claims 149-153 or 164-165, or a pharmaceutically acceptable salt or deuterated form thereof, wherein R⁰ is

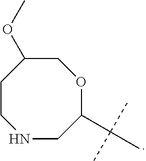

168. The compound of any one of claims 149-153 or 164-165, or a pharmaceutically acceptable salt or deuterated form thereof, wherein R⁰ is

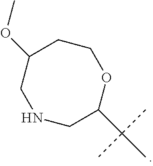

169. The compound of claim 164, or a pharmaceutically acceptable salt or deuterated form thereof, wherein R⁰ is

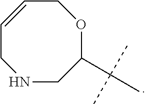

170. The compound of claim 164, or a pharmaceutically acceptable salt or deuterated form thereof, wherein R⁰ is

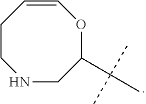

171. The compound of claim 164, or a pharmaceutically acceptable salt or deuterated form thereof, wherein R⁰ is

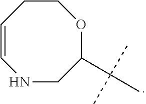

172. The compound of any one of claims 1-145 or 149, or a pharmaceutically acceptable salt or deuterated form thereof, wherein R⁰ is

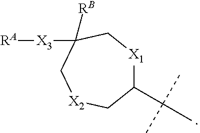

173. The compound of claim 172, or a pharmaceutically acceptable salt or deuterated form thereof, wherein R⁰ is HN.

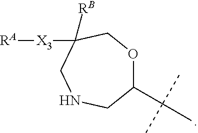

174. The compound of any one of claims 149 or 172-173, or a pharmaceutically acceptable salt or deuterated form thereof, wherein R^{A} is H or C₁₋₆alkyl.

175. The compound of claim 174, or a pharmaceutically acceptable salt or deuterated form thereof, wherein R^{A} is H.

176. The compound of claim 174, or a pharmaceutically acceptable salt or deuterated form thereof, wherein R^{A} is methyl.

177. The compound of any one of claims 149 or 172-176, or a pharmaceutically acceptable salt or deuterated form thereof, wherein R^{B} is C₁₋₆alkyl, C₂₋₆ alkenyl, C₁₋₆alkylene-carbocyclyl, or C₁₋₆alkylene-heteroaryl.

178. The compound of claim 177, or a pharmaceutically acceptable salt or deuterated form thereof, wherein R^{B} is C₁₋₆alkyl, C₁₋₆alkylene-aryl or —C₁₋₆alkylene-5-6 membered heteroaryl.

179. The compound of claim 172 or 173, or a pharmaceutically acceptable salt or deuterated form thereof, wherein R⁰ is

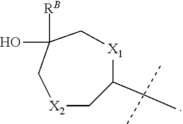

180. The compound of claim 172 or 173, or a pharmaceutically acceptable salt or deuterated form thereof, wherein R⁰ is

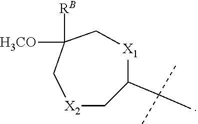

181. The compound of claim 172 or 173, or a pharmaceutically acceptable salt or deuterated form thereof, wherein R⁰ is

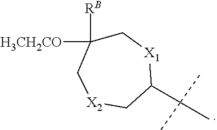

182. The compound of any one of claims 177-181, or a pharmaceutically acceptable salt or deuterated form thereof, wherein R^{B} is methyl.

183. The compound of any one of claims 177-181, or a pharmaceutically acceptable salt or deuterated form thereof, wherein R^{B} is ethyl.

184. The compound of any one of claims 177-181, wherein R^{B} is H.

185. The compound of claim 180, or a pharmaceutically acceptable salt or deuterated form thereof, wherein R⁰ is

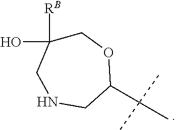

186. The compound of claim 181, or a pharmaceutically acceptable salt or deuterated form thereof, wherein R⁰ is

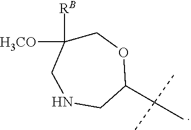

187. The compound of claim 182, or a pharmaceutically acceptable salt or deuterated form thereof, wherein R⁰ is

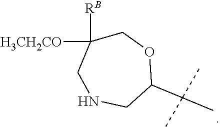

188. The compound of any one of claims 185-187, or a pharmaceutically acceptable salt or deuterated form thereof, wherein R^{B} is methyl.

189. The compound of any one of claims 185-187, or a pharmaceutically acceptable salt or deuterated form thereof, wherein R^{B} is ethyl.

190. The compound of any one of claims 172-178, or a pharmaceutically acceptable salt or deuterated form thereof, wherein X₃ is O.

191. The compound of any one of claims 1-145, or a pharmaceutically acceptable salt or deuterated form thereof, wherein R⁰ is

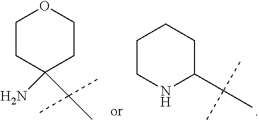

192. The compound of any one of claims 127-191, or a pharmaceutically acceptable salt or deuterated form thereof, wherein R⁶ is C₁₋₃ alkyl.

193. The compound of claim 192, or a pharmaceutically acceptable salt or deuterated form thereof, wherein R⁶ is CH₃.

194. The compound of any one of claims 127-193, or a pharmaceutically acceptable salt or deuterated form thereof, wherein R⁷ is H.

195. The compound of any one of claims 127-194, or a pharmaceutically acceptable salt or deuterated form thereof, wherein R¹⁰ is —H or —F.

196. The compound of claim 1, or a pharmaceutically acceptable salt or deuterated form thereof, wherein the compound is selected from Table 1.

197. A pharmaceutical composition comprising an effective amount of a compound of any one of claims 1-196, or a pharmaceutically acceptable salt or deuterated form thereof and a pharmaceutically acceptable adjuvant, diluent or carrier.

198. A method for treating an obstructive disease of the airway in a patient in need thereof, comprising, administering to the patient an effective amount of a compound of any one of claims 1-196, or the composition of claim 197.

199. The method of claim 198, wherein the obstructive disease of the airway is asthma, chronic obstructive pulmonary disease (COPD), bronchitis, emphysema, cystic fibrosis (CF), bronchiectasis, sarcoidosis, alpha-1 antitrypsin (A1AT) deficiency, farmer's lung and related diseases, hypersensitivity pneumonitis, lung fibrosis, complications of lung transplantation, vasculitic and thrombotic disorders of the lung vasculature, pulmonary hypertension, antitussive activity including treatment of chronic cough associated with inflammatory and secretory conditions of the airways, iatrogenic cough, acute and chronic rhinitis including rhinitis medicamentosa, and vasomotor rhinitis; perennial and seasonal allergic rhinitis including rhinitis nervosa (hay fever), nasal polyposis; acute viral infection including the common cold, and infection due to a respiratory virus, acute lung injury, or acute respiratory distress syndrome (ARDS).

200. The method of claim 199, wherein the obstructive disease of the airway is asthma.

201. The method of claim 199, wherein the obstructive disease of the airway is acute respiratory distress syndrome (ARDS).

202. The method of claim 199, wherein the obstructive disease of the airway is bronchitis.

203. The method of claim 199, wherein the obstructive disease of the airway is lung fibrosis.

204. The method of claim 199, wherein the obstructive disease of the airway is emphysema.

205. The method of claim 199, wherein the obstructive disease of the airway is cystic fibrosis (CF).

206. The method of claim 199, wherein the obstructive disease of the airway is bronchiectasis.

207. The method of claim 199, wherein the obstructive disease of the airway is sarcoidosis.

208. The method of claim 199, wherein the obstructive disease of the airway is alpha-1 antitrypsin (A1AT) deficiency.

209. The method of claim 199, wherein the obstructive disease of the airway is farmer's lung.

210. The method of claim 199, wherein the obstructive disease of the airway is hypersensitivity pneumonitis.

211. The method of claim 199, wherein the obstructive disease of the airway is a complication of lung transplantation.

212. The method of claim 199, wherein the obstructive disease of the airway is a vasculitic or thrombotic disorder of the lung vasculature.

213. The method of claim 199, wherein the obstructive disease of the airway is pulmonary hypertension.

214. The method of claim 199, wherein the obstructive disease of the airway is iatrogenic cough.

215. The method of claim 199, wherein the obstructive disease of the airway is acute rhinitis.

216. The method of claim 199, wherein the obstructive disease of the airway is chronic rhinitis.

217. The method of claim 199, wherein the obstructive disease of the airway is rhinitis medicamentosa or vasomotor rhinitis.

218. The method of claim 199, wherein the obstructive disease of the airway is nasal polyposis.

219. The method of claim 199, wherein the obstructive disease of the airway is COPD.

220. The method of claim 200, wherein the asthma is bronchial, allergic, intrinsic, extrinsic, exercise-induced or drug-induced asthma.

221. The method of claim 200, wherein the bronchitis is infectious bronchitis or eosinophilic bronchitis.

222. The method of claim 203, wherein the lung fibrosis is idiopathic pulmonary fibrosis, cryptogenic fibrosing alveolitis, idiopathic interstitial pneumonia, or fibrosis complicating anti-neoplastic therapy or chronic infection.

223. The method of claim 206, wherein the bronchiectasis is non-cystic fibrosis bronchiectasis (NCFBE).

224. The method of claim 206, wherein the bronchiectasis is associated with cystic fi25.

225. The method of claim 213, wherein the pulmonary hypertension is pulmonary arterial hypertension.

226. The method of claim 213, wherein the pulmonary hypertension is pulmonary hypertension due to left heart disease.

227. The method of claim 213, wherein the pulmonary hypertension is pulmonary hypertension associated with chronic lung disease.

228. A method for treating cystic fibrosis in a patient in need thereof, comprising, administering to the patient an effective amount of a compound of any one of claims 1-196, or the composition of claim 197.

229. The method of claim 228, wherein the treating comprises improving the lung function of the patient, as compared to the lung function of the patient prior to treatment.

230. The method of claim 229, wherein improving lung function of the patient comprises increasing the patient's forced expiratory volume in 1 second (FEV₁), increasing the patient's forced vital capacity (FVC), increasing the patient's peak expiratory flow rate (PEFR), or increasing the patient's forced expiratory flow between 25% and 75% of FVC (FEF_{(25-75%)}), as compared to the respective value for the patient prior treatment.

231. The method of claim 229 or 230, wherein the lung function is measured by spirometry.

232. A method for treating bronchiectasis in a patient in need thereof, comprising, administering to the patient, an effective amount of a compound of any one of claims 1-196, or the composition of claim 197.

233. The method of claim 232, wherein the bronchiectasis is non-cystic fibrosis bronchiectasis (NCFBE).

234. The method of claim 232, wherein the bronchiectasis is associated with cystic fibrosis.

235. The method of any one of claims 232-234, wherein treating comprises improving the lung function of the patient, as compared to the lung function of the patient prior to treatment.

236. The method of claim 235, wherein improving lung function of the patient comprises increasing the patient's forced expiratory volume in 1 second (FEV₁), increasing the patient's forced vital capacity (FVC), increasing the patient's peak expiratory flow rate (PEFR), or increasing the patient's forced expiratory flow between 25% and 75% of FVC (FEF_{(25-75%)}), as compared to the respective value for the patient prior to treatment.

237. The method of claim 235-236, wherein the lung function is measured by spirometry.

238. The method of any one of claims 232-234, wherein treating comprises decreasing the rate of pulmonary exacerbation, as compared to the rate of pulmonary exacerbation of the patient prior to treatment.

239. The method of any one of claims 232-238, wherein treating comprises increasing the time to first pulmonary exacerbation, as compared to an untreated patient.

240. The method of claim 238 or 239, wherein the pulmonary exacerbation is characterized by three or more of the following symptoms exhibited for at least 48 hours by the patient: (1) increased cough; (2) increased sputum volume or change in sputum consistency; (3) increased sputum purulence; (4) increased breathlessness and/or decreased exercise tolerance; (5) fatigue and/or malaise; (6) hemoptysis.

241. A method for treating chronic rhinosinusitis (CRS) in a patient in need thereof, comprising, administering to the patient, an effective amount of a compound of any one of claims 1-196, or the composition of claim 197.

242. The method of claim 241, wherein the chronic rhinosinusitis is chronic rhinosinusitis without nasal polyps (CRSsNP).

243. The method of claim 241, wherein the chronic rhinosinusitis is chronic rhinosinusitis with nasal polyps (CRSwNP).

244. The method of any one of claims 241-242, wherein the chronic rhinosinusitis is refractory chronic rhinosinusitis.

245. The method of any one of claims 241-244, wherein treating comprises reducing, diminishing the severity of, delaying the onset of, or eliminating one or more symptoms of CRS.

246. The method of claim 245, wherein the one or more symptoms of CRS is nasal congestion; nasal obstruction; nasal discharge; post-nasal drip; facial pressure; facial pain; facial fullness; reduced smell; depression; mucosal edema; mucopurulent discharge; obstruction of the middle meatus; mucosal changes within the ostiomeatal complex and sinuses; or rhinorrhea.

247. A method for treating hidradenitis suppurativa (HS) in a patient in need thereof, comprising, administering to the patient, an effective amount of a compound of any one of claims 1-196, or the composition of claim 197.

248. The method of claim 247, wherein the hidradenitis suppurativa (HS) is Hurley stage I.

249. The method of claim 247, wherein the hidradenitis suppurativa (HS) is Hurley stage II.

250. The method of claim 247, wherein the hidradenitis suppurativa (HS) is Hurley stage III.

251. A method for treating cancer in a patient in need thereof, comprising, administering to the patient, an effective amount of a compound of any one of claims 1-196, or the composition of claim 197.

252. The method of claim 251, wherein the cancer is a metastatic cancer.

253. The method of claim 252, wherein the metastatic cancer is breast to lung metastatic cancer.

254. The method of claim 252, wherein the metastatic cancer comprises metastasis of breast cancer to the brain, bone, pancreas, lymph nodes or liver.

255. The method of claim 252, wherein the metastatic cancer comprises metastasis of bone cancer to the lung.

256. The method of claim 252, wherein the metastatic cancer comprises metastasis of colorectal cancer to the peritoneum, the pancreas, the stomach, the lung, the liver, the kidney, or the spleen.

257. The method of claim 252, wherein the metastatic cancer comprises metastasis of stomach cancer to the mesentery, the spleen, the pancreas, the lung, the liver, the adrenal gland, or the ovary.

258. The method of claim 252, wherein the metastatic cancer comprises metastasis of liver cancer to the intestine, spleen, pancreas, stomach, lung, or the kidney.

259. The method of claim 252, wherein the metastatic cancer comprises metastasis of lymphoma to the kidney, ovary, liver, bladder, or the spleen.

260. A method for treating lupus nephritis in a patient in need thereof, comprising, administering to the patient, an effective amount of a compound of any one of claims 1-196, or the composition of claim 197.

261. A method for treating rheumatoid arthritis in a patient in need thereof, comprising, administering to the patient, an effective amount of a compound of any one of claims 1-196, or the composition of claim 197.

262. A method for treating inflammatory bowel disease (IBD) in a patient in need thereof, comprising, administering to the patient, an effective amount of a compound of any one of claims 1-196, or the composition of claim 197.

263. The method of claim 262, wherein the inflammatory bowel disease (IBD) is Crohn's disease.

264. The method of claim 262, wherein the inflammatory bowel disease (IBD) is ulcerative colitis.

265. A method for treating an anti-neutrophil cytoplasmic antibody (ANCA) associated vasculitis in a patient in need thereof, comprising, administering to the patient, an effective amount of a compound of any one of claims 1-196, or the composition of claim 197.

266. The method of claim 265, wherein the ANCA associated disease is granulomatosis with polyangiitis (GPA).

267. The method of claim 265, wherein the ANCA associated disease is microscopic polyangiitis (MPA).

268. A method for treating a disease in a patient in need thereof comprising, administering to the patient, an effective amount of a compound of any one of claims 1-196, or the composition of claim 197, wherein the disease is giant cell arteritis, polyarteritis nodosa, anti-GBM disease (Goodpasture's), systemic scleroderma, diabetic nephropathy, diabetic neuropathy, diabetic retinopathy, diabetic ulcers, Duchenne muscular dystrophy, bronchiolitis obliterans, atopic dermatitis, pyoderma gangrenosum, sweet's syndrome, dermatomyositis/polymyositis, neutrophilic dermatoses, thrombosis, bronchopulmonary dysplasia, amyotrophic lateral sclerosis, sickle cell anemia, psoriasis, or a ventilator-induced lung injury.

269. A method for treating a heart failure in a patient in need thereof, comprising administering to the patient, an effective amount of a compound of any one of claims 1-196, or the composition of claim 197.

270. The method of claim 269, wherein the heart failure is heart failure with reduced ejection fraction.

271. The method of claim 269, wherein the heart failure is heart failure with preserved ejection fraction.

272. A compound selected from Table 1, or a pharmaceutically acceptable salt or deuterated form thereof.
